(19) United States
(12) Patent Application Publication
(10) Pub. No.: US 2012/0208781 A1
(43) Pub. Date: Aug. 16, 2012

(54) AMINOGLYCOSIDE DOSING REGIMENS

(75) Inventors: Jon B. Bruss, Cincinnati, OH (US); Corwin F. Kostrub, Belmont, CA (US); Eliana Saxon Armstrong, San Mateo, CA (US); Robert T. Cass, Fairfax, CA (US); George H. Miller, Menlo Park, CA (US); James Bradley Aggen, Burlingame, CA (US); Adam Aaron Goldblum, Berkeley, CA (US); Paola Dozzo, San Francisco, CA (US); Martin Sheringham Linsell, San Mateo, CA (US)

(73) Assignee: Achaogen, Inc., South San Francisco, CA (US)

(21) Appl. No.: 13/294,429

(22) Filed: Nov. 11, 2011

Related U.S. Application Data
(63) Continuation of application No. PCT/US2010/035006, filed on May 14, 2010.
(60) Provisional application No. 61/178,470, filed on May 14, 2009.
(60) Provisional application No. 61/181,619, filed on May 27, 2009.
(60) Provisional application No. 61/241,355, filed on Sep. 10, 2009.
(60) Provisional application No. 61/313,057, filed on Mar. 11, 2010.
(60) Provisional application No. 61/178,809, filed on May 15, 2009.
(60) Provisional application No. 61/312,349, filed on Mar. 10, 2010.
(60) Provisional application No. 61/178,814, filed on May 15, 2009.
(60) Provisional application No. 61/312,351, filed on Mar. 10, 2010.
(60) Provisional application No. 61/178,826, filed on May 15, 2009.
(60) Provisional application No. 61/312,353, filed on Mar. 10, 2010.
(60) Provisional application No. 61/178,854, filed on May 15, 2009.
(60) Provisional application No. 61/312,354, filed on Mar. 10, 2010.
(60) Provisional application No. 61/178,834, filed on May 15, 2009.
(60) Provisional application No. 61/312,356, filed on Mar. 10, 2010.

(51) Int. Cl. A61K 31/7036; A61K 31/7052; A61K 31/7056; A61P 31/04; A61K 31/706

(52) U.S. Cl. 514/41; 514/40

(57) ABSTRACT

The present invention provides new aminoglycoside dosing regimens associated with enhanced microbicidal activity and reduced nephrotoxicity, as well as methods of using these dosing regimens to treat various bacterial infections.

### CROSS-REFERENCE TO RELATED APPLICATIONS

This application is a continuation of International PCT Application No. PCT/US2010/035006, filed May 14, 2010, now pending, which claims the benefit under 35 U.S.C. §119(e) of U.S. Provisional Patent Application No. 61/178,470 filed May 14, 2009; U.S. Provisional Patent Application No. 61/181,619 filed May 27, 2009; U.S. Provisional Patent Application No. 61/241,355 filed Sep. 10, 2009; U.S. Provisional Patent Application No. 61/313,057 filed Mar. 11, 2010; U.S. Provisional Patent Application No. 61/178,809 filed May 15, 2009; U.S. Provisional Patent Application No. 61/312,349 filed Mar. 10, 2010; U.S. Provisional Patent Application No. 61/178,814 filed May 15, 2009; U.S. Provisional Patent Application No. 61/312,351 filed Mar. 10, 2010; U.S. Provisional Patent Application No. 61/178,826 filed May 15, 2009; U.S. Provisional Patent Application No. 61/312,353 filed Mar. 10, 2010; U.S. Provisional Patent Application No. 61/178,854 filed May 15, 2009; U.S. Provisional Patent Application No. 61/312,354 filed Mar. 10, 2010; U.S. Provisional Patent Application No. 61/178,834 filed May 15, 2009; and U.S. Provisional Patent Application No. 61/312,356 filed Mar. 10, 2010. The foregoing applications are incorporated herein by reference in their entireties.

### BACKGROUND

1. Technical Field

The present invention relates to aminoglycosides and their use in treating infections using dosing regimens that are effective, and are associated with reduced potential for nephrotoxicity.

2. Description of the Related Art

Aminoglycoside antibiotics are a well-known class of antibiotics with an established record of both efficacy and safety. The primary use-limiting adverse reactions associated with the class are nephrotoxicity and less commonly ototoxicity. Reports of nephrotoxicity generally range from 5% to 30%, mostly from studies involving multiple daily doses of aminoglycosides and in patients with other contributing factors. Overall, the risk of nephrotoxicity from aminoglycosides is estimated at approximately 10%. Given their wide use, it is not surprising that aminoglycosides are among the more frequently reported drug-related causes of nephrotoxicity. If it occurs, the resulting renal impairment typically appears several days (7-10 days) after initiation of treatment. This is usually mild and reversible after drug withdrawal, although it may be more severe, especially in patients with underlying renal disease. It is non-oliguric in nature, and is associated with a gradual rise in serum creatine (Cr) and blood urea nitrogen (BUN).

Aminoglycoside-associated nephrotoxicity is dependent on aminoglycoside uptake and accumulation into the renal tubule epithelial cells. In the kidneys, aminoglycosides accumulate preferentially in the proximal tubule, leading to eventual cell destruction. Aminoglycosides are filtered by the renal glomerulus into the proximal tubule of the kidney, where they are taken up into proximal tubule cells, Uptake is believed to involve binding to the brush border and transport into the cell through an active process of pinocytosis, primarily mediated by the transporter, megalin, a low-density lipoprotein receptor-related protein-2 expressed on the brush border. The rate for megalin-mediated uptake of aminoglycosides is relatively fast, while the clearance half-life from the renal cells is considerably slower, leading to accumulation of drug in the kidney and resulting nephrotoxicity.

Ototoxicity is another potential toxicity associated with the aminoglycoside class of antibiotics. A review of 1976 patients receiving gentamicin or another similar (aminoglycoside) antibiotic showed that about 3% developed some sort of vestibular injury (Kahlmeter and Dahlager, 1982, J Antimicrob Chemother. 1984:13 (suppl A): 9-22). Ototoxicity can be either auditory or vestibular and is generally correlated with longer durations of therapy and total cumulative doses, particularly total cumulative AUC. The mechanism of aminoglycoside ototoxicity is unknown, but it has been postulated to involve both apoptotic pathways and the formation of free radicals (reviewed in Forge and Schacht, 2000, Audio Neurootol 5:3-22). It has also been suggested that the mechanism of ototoxicity is through reduction of mitochondrial protein synthesis (Guan et al., 2000, Human Mol Gen 9, 12, 1787-93).

Aminoglycosides vary with respect to their potency against various bacterial strains. In some cases, certain aminoglycosides that have relatively high potency, for example, against a broad spectrum of bacteria or against certain drug-resistant bacteria, are, unfortunately, relatively nephrotoxic. Conversely, some aminoglycosides that are less nephrotoxic are, nonetheless, also less potent against important bacterial strains.

Given the importance of aminoglycosides in treating multiple human infections, there is clearly a need in the art for new aminoglycosides having reduced nephrotoxicity, as well as new dosing regimens effective for treating bacterial infections, while mitigating the potential for nephrotoxicity associated with both existing and new aminoglycosides. There is also a need in the art for new aminoglycosides having reduced ototoxicity.

### BRIEF SUMMARY

The present invention provides new dosing regimens for treating bacterial infections using an aminoglycoside, which are associated with enhanced efficacy and reduced risk of nephrotoxicity. The invention is directed, in a first aspect, to methods for treating a bacterial infection in a human subject. In a second aspect, the invention is directed to aminoglycosides for use in the treatment of a bacterial infection in a human subject, and aminoglycosides prepared for use in the treatment of a bacterial infection in a human subject. In addition, in a third aspect, the invention is directed to the use of aminoglycosides for the manufacture of a medicament for the treatment of a bacterial infection in a human subject, and the use of aminoglycosides for the manufacture of a medicament prepared for the treatment of a bacterial infection in a human subject.

In one first general embodiment of the first aspect of the invention, the present invention includes a method for treating a bacterial infection in a human subject, the method comprising administering an effective amount of an aminoglycoside to the subject not more than once per day for not more than five days, the effective amount being a potency-normalized amount of at least N_{GEN}×9 mg/kg/day, where N_{GEN}=MIC_{AG}/MIC_{GEN} is a normalizing factor defined by the ratio of a minimum inhibitory concentration (e.g., a minumum inhibitory concentration (90%)) of the administered aminoglycoside, MIC_{AG}, to a minimum inhibitory concentration (e.g., a minumum inhibitory concentration (90%)) of gentamicin, MIC_{GEN}. The minimum inhibitory concentration (e.g., a mininmum inhibitory concentration (90%)) of the administered aminoglycoside and gentamicin can be for the bacteria type (e.g., species or strain) infecting the subject. The effective amount may alternatively be a potency-normalized amount of at least N_{GEN}×7 mg/kg/day or at least N_{GEN}×8 mg/kg/day. The effective amount may alternatively be a potency-normalized amount ranging from about N_{GEN}×9 mg/kg/day to about N_{GEN}×15 mg/kg/day, ranging from about N_{GEN}×8 mg/kg/day to about N_{GEN}×15 mg/kg/day, or ranging from about N_{GEN}×7 mg/kg/day to about N_{GEN}×15 mg/kg/day. Alternatively, the effective amount may be a potency-normalized amount ranging from about N_{GEN}×9 mg/kg/day to about N_{GEN}×30 mg/kg/day, ranging from about N_{GEN}×8 mg/kg/day to about N_{GEN}×30 mg/kg/day, or ranging from about N_{GEN}×7 mg/kg/day to about N_{GEN}×30 mg/kg/day. In particular embodiments, the aminoglycoside may be administered to the subject as described herein, including administered for 1 to 5 days, 2 to 5 days, 3 to 5 days, or 4 to days. In particular embodiments, the effective amount administered can be a potency-normalized amount of about N_{GEN}×15 mg/kg/day.

In another second general embodiment of the first aspect of the invention, the present invention provides a method for treating a bacterial infection in a subject, the method comprising administering an effective amount of an aminoglycoside to the subject not more than once per day for not more than five days, the effective amount being equal to or less than a toxicity-normalized amount of T_{GEN}×50 mg/kg/day, where T_{GEN}=MTC_{AG}/MTC_{GEN} is a normalizing factor defined by the ratio of a minimum toxic concentration of the administered aminoglycoside, MTC_{AG}, to a minimum toxic concentration of gentamicin, MTC_{GEN}. The effective amount may be equal to or less than a toxicity-normalized amount of T_{GEN}×40 mg/kg/day, or T_{GEN}×30 mg/kg/day. In certain embodiments, the effective amount may be equal to or less than a toxicity normalized amount ranging from T_{GEN}×30 mg/kg/day to T_{GEN}×50 mg/kg/day or ranging from T_{GEN}×15 mg/kg/day to T_{GEN}×50 mg/kg/day. In certain embodiments, the effective amount may be a toxicity-normalized amount ranging from T_{GEN}×30 mg/kg/day to T_{GEN}×50 mg/kg/day, ranging from T_{GEN}×15 mg/kg/day to T_{GEN}×50 mg/kg/day, ranging from T_{GEN}×7 mg/kg/day to T_{GEN}×50 mg/kg/day, ranging from T_{GEN}×9 mg/kg/day to T_{GEN}×50 mg/kg/day, ranging from T_{GEN}×12 mg/kg/day to T_{GEN}×50 mg/kg/day, ranging from T_{GEN}×7 mg/kg/day to T_{GEN}×100 mg/kg/day, or ranging from T_{GEN}×15 mg/kg/day to T_{GEN}×100 mg/kg/day. In particular embodiments, the aminoglycoside may be administered to the subject as described herein, including administered for 1 to 5 days, 2 to 5 days, 3 to 5 days, or 4 to 5 days.

In a third general embodiment, related to the first and second general embodiments of the first aspect of the invention, the present invention provides a method for treating a bacterial infection in a subject, the method comprising administering an effective amount of an aminoglycoside to the subject not more than once per day for not more than five days, wherein the effective amount is equal to or greater than a potency-normalized amount of N_{GEN}×9 mg/kg/day and the effective amount is equal to or less than a toxicity-normalized amount of T_{GEN}×50 mg/kg/day. The potency-normalized amounts, the toxicity-normalized amounts, and number of days of administration to the subject in this third general embodiment can be alternatively, additionally or more particularly as described herein, including as described in connection with the first and the second general embodiments of the first aspect of the invention.

In another fourth general embodiment of the first aspect of the invention, the present invention includes a method for treating a bacterial infection in a human subject, the method comprising administering an effective amount of an aminoglycoside to the subject not more than once per day for not more than five days to achieve a maximum serum concentration of the administered aminoglycoside, Cₘₐₓ, equal to at least 5 times the minimum inhibitory concentration (e.g., a minumum inhibitory concentration (90%)) of the administered aminoglycoside, MIC_{AG}, for the bacterial type (i.e., species or strain) infecting the subject. In particular embodiments, the Cₘₐₓ is equal to at least 8 times the minimum inhibitory concentration (e.g., a minumum inhibitory concentration (90%)) of the administered aminoglycoside, MIC_{AG}, for the bacterial type (i.e., species or strain) infecting the subject. In another embodiment, the Cₘₐₓ is in the range of about 5 to about 150 times the minimum inhibitory concentration (e.g., a minumum inhibitory concentration (90%)) of the administered aminoglycoside, MIC_{AG}, for the bacterial type (i.e., species or strain) infecting the subject. In various embodiments, the aminoglycoside is administered to the subject as described herein, including for 1 to 5 days, 2 to 5 days, 3 to 5 days, or 4 to 5 days.

In a further fifth general embodiment of the first aspect of the invention, the present invention includes a method for treating a bacterial infection in a human subject, the method comprising administering an effective amount of an aminoglycoside to the subject not more than once per day for not more than five days to achieve a maximum serum concentration of the administered aminoglycoside, Cₘₐₓ, and a pharmacokinetic profile (e.g., a serum pharmacokinetic profile) defined by a time-concentration curve, the ratio of Cₘₐₓ to total area under the time-concentration curve, AUC, being at least 0.4 hr⁻¹. In particular embodiments, the ratio of Cₘₐₓ to total area under the time-concentration curve, AUC, is at least 0.6 hr⁻¹. In another embodiment, the ratio of Cₘₐₓ to total area under the time-concentration curve, AUC, ranges from about 0.4 to about 1.0 hr⁻¹. In various embodiments, the aminoglycoside is administered to the subject as described herein, including for 1 to 5 days, 2 to 5 days, 3 to 5 days, or 4 to 5 days.

In a further sixth general embodiment of the first aspect of the invention, the present invention includes a method for treating a bacterial infection in a human subject, the method comprising administering an effective amount of an aminoglycoside to the subject not more than once per day for not more than five days to achieve a pharmacokinetic profile (e.g., a serum pharmacokinetic profile) defined by a time-concentration curve, at least 30% of total area under the time-concentration curve, AUC, being an area above a kidney saturation concentration, C_{KS}, for the aminoglycoside. C_{KS} is a serum concentration that corresponds to the level at which uptake into the proximal tubule cell is maximal or saturated for the aminoglycoside. In various embodiments, the aminoglycoside is administered to the subject as described herein, including for 1 to 5 days, 2 to 5 days, 3 to 5 days, or 4 to 5 days.

In a seventh general embodiment of the first aspect of the invention, the present invention includes a method for treating a bacterial infection in a human subject, the method comprising administering an effective amount of an aminoglycoside to the subject not more than once per day for at least two days by intravenous infusion over an infusion period of less than or equal to about 15 minutes. In particular embodiments, the aminoglycoside is administered over an infusion period of not more than about ten minutes. In other particular embodiments, the aminoglycoside is administered to the subject over an infusion period of less than or equal to about one hour, or less than or equal to 30 minutes. In another embodiment, the aminoglycoside is administered to the subject over an infusion period ranging from about 5 minutes to about 60 minutes. Alternatively, the aminoglycoside may be administered to the subject over an infusion period ranging from about 5 minutes to about 30 minutes, or ranging from about 5 minutes to about 15 minutes, or ranging from about 5 minutes to about 10 minutes. In further embodiments, the aminoglycoside is administered to the subject as described herein, including for 2 to 6 days, 2 to 5 days, 2 to 4 days, or 2 to 3 days.

In another eighth general embodiment, related to the seventh general embodiment of the invention, the present invention includes a method for treating a bacterial infection in a human subject, the method comprising administering an effective amount of an aminoglycoside to the subject not more than once per day for at least two days by intravenous infusion with an infusion rate of at least N_{GEN}×0.3 mg/kg/min, where N_{GEN}=MIC_{AG}/MIC_{GEN} is a normalizing factor defined by the ratio of a minimum inhibitory concentration (e.g., a minumum inhibitory concentration (90%)) of the administered aminoglycoside, MIC_{AG}, to a minimum inhibitory concentration (e.g., a minumum inhibitory concentration (90%)) of gentamicin, MIC_{GEN}, for the bacterial type (i.e., species or strain) infecting the subject. In particular embodiments, the infusion rate is at least N_{GEN}×0.5 mg/kg/min. In another embodiment, the infusion rate ranges from about N_{GEN}×0.3 mg/kg/min to about N_{GEN}×1.0 mg/kg/min. In particular embodiments, the aminoglycoside is administered over an infusion period as described herein, including as described in connection with the seventh general embodiment of the first aspect of the invention, such as, for example, an infusion period of not more than about 10 minutes. In further embodiments, the aminoglycoside is administered to the subject as described herein, including for 2 to 6 days, 2 to 5 days, 2 to 4 days, or 2 to 3 days.

In another ninth general embodiment, the present invention provides a method for treating a bacterial infection in a human subject, the method comprising administering an effective amount of an aminoglycoside to the subject by intraveneous infusion, the infused amount being a potency-normalized amount of at least N_{GEN}×12 mg/kg/infusion, where N_{GEN}=MIC_{AG}/MIC_{GEN} is a normalizing factor defined by the ratio of a minimum inhibitory concentration (e.g., a minumum inhibitory concentration (90%)) of the administered aminoglycoside, MIC_{AG}, to a minimum inhibitory concentration (e.g., a minumum inhibitory concentration (90%)) of gentamicin, MIC_{GEN}, for the bacterial type (i.e., species or strain) infecting the subject. In a particular embodiment, the infused amount is a potency-normalized amount of at least N_{GEN}×15 mg/kg/infusion. In other embodiments, the infused amount is a potency-normalized amount ranging from about N_{GEN}×12 mg/kg/infusion to about N_{GEN}×50 mg/kg/infusion. In particular embodiments, the aminoglycoside is administered over an infusion period as described herein, including as described in connection with the seventh general emodiment of the first aspect of the invention, such as for example an infusion period of not more than 30 minutes. In further embodiments, the aminoglycoside is administered to the subject as described herein, including for 2 to 6 days, 2 to 5 days, 2 to 4 days, or 2 to 3 days.

In another tenth general embodiment, related to the fourth and seventh through ninth general embodiments of the first aspect of the invention, the present invention includes a method for treating a bacterial infection in a human subject, the method comprising administering an effective amount of an aminoglycoside to the subject by intravenous infusion to achieve a maximum serum concentration of the administered aminoglycoside, Cₘₐₓ, equal to at least 5 times the minimum inhibitory concentration (e.g., a minumum inhibitory concentration (90%)) of the administered aminoglycoside, MIC_{AG}, for the bacterial type (i.e., species or strain) infecting the subject. In particular embodiments, the Cₘₐₓ is equal to at least 8 times the MIC_{AG}, for the bacterial type (i.e., species or strain) infecting the subject. In other embodiments, the Cₘₐₓ ranges from about 5 to about 150 times the MIC_{AG}, for the bacterial type (e.g., species or strain) infecting the subject. This general embodiment can alternatively, additionally or more particularly include other features as described herein, including as described in connection with the fourth and seventh through ninth general emodiments of the first aspect of the invention.

In an eleventh general embodiment, related to the fifth and seventh through ninth general embodiments of the first aspect of the invention, the present invention includes a method for treating a bacterial infection in a human subject, the method comprising administering an effective amount of an aminoglycoside to the subject by intravenous infusion to achieve a maximum serum concentration of the administered aminoglycoside, Cₘₐₓ, and a pharmacokinetic profile (e.g., a serum pharmacokinetic profile) defined by a time-concentration curve, the ratio of Cₘₐₓ to total area under the time-concentration curve, AUC, being at least 0.4 hr⁻¹. In a particular embodiment, the ratio of Cₘₐₓ to total area under the time-concentration curve, AUC, is at least 0.6 hr⁻¹. In other embodiments, the ratio of Cₘₐₓ to total area under the time-concentration curve, AUC, is about 0.4 to about 1.0 hr⁻¹. This general embodiment can alternatively, additionally or more particularly include other features as described herein, including as described in connection with the fifth and seventh through ninth general emodiments of the first aspect of the invention.

In a further twelvth general embodiment, related to the sixth through ninth general embodiments of the first aspect of the invention, the present invention includes a method for treating a bacterial infection in a human subject, the method comprising administering an effective amount of an aminoglycoside to the subject by intravenous infusion to achieve a serum pharmacokinetic profile defined by a time-concentration curve, at least 30% of total area under the time-concentration curve, AUC, being an area above a kidney saturation concentration, C_{KS}, for the aminoglycoside. This general embodiment can alternatively, additionally or more particularly include other features as described herein, including as described in connection with the sixth through ninth general emodiments of the first aspect of the invention.

In another thirteenth general embodiment of the first aspect of the invention, the present invention includes a method for treating a bacterial infection in a human subject, the method comprising administering an effective amount of an aminoglycoside to the subject not more than once per day for not more than five days, and maintaining substantially baseline renal function as indicated by one or more nephrotoxicity biomarkers. In one embodiment, one of the one or more nephrotoxicity markers is a glomerular filtration rate (GFR), a blood urea nitrogen (BUN) level, a serum creatine level, or a creatine clearance rate. In various embodiments, the aminoglycoside is administered to the subject as described herein, including for 1 to 5 days, 2 to 5 days, 3 to 5 days, or 4 to 5 days.

In another fourteenth general embodiment of the first aspect of the invention, the present invention provides a method for treating a bacterial infection in a human subject without causing otoxicity, the method comprising administering an effective amount of an aminoglycoside to the subject to achieve a maximum serum concentration of the administered aminoglycoside, Cₘₐₓ, equal to at least 8 times the minimum inhibitory concentration (e.g., a minumum inhibitory concentration (90%)) of the administered aminoglycoside, MIC_{AG}, for the bacterial type (i.e., species or strain) infecting the subject, wherein preferably, the aminoglycoside is administered once per day for at least five days, once per day for at least 7 days, once per day for at least 10 days, or once per days for at least 14 days.

In a fifteenth general embodiment, related to the fourteenth general embodiment of the first aspect of the invention, the present invention also includes a method for treating a bacterial infection in a human subject, the method comprising administering an effective amount of an aminoglycoside to the subject to achieve a maximum serum concentration of the administered aminoglycoside, Cₘₐₓ, equal to at least 8 times the minimum inhibitory concentration (e.g., a minumum inhibitory concentration (90%)) of the administered aminoglycoside, MIC_{AG}, for the bacterial type (i.e., species or strain) infecting the subject at least once per day, and maintaining substantially baseline auditory function as indicated by one or more auditory markers. The auditory markers can be an auditory brainstem response (ABR). In various such embodiments, the aminoglycoside may be administered not more than once per day. In various such embodiments, the Cₘₐₓ ranges from about 8 to about 150 times the minimum inhibitory concentration of the administered aminoglycoside, MIC_{AG}, for the bacterial type (i.e., species or strain) infecting the subject. In various embodiments, the aminoglycoside can be administered to the subject as described herein, including for 1 to 5 days, 2 to 5 days, 3 to 5 days, or 4 to 5 days, or including alternatively, once per day for at least five days, once per day for at least 7 days, once per day for at least 10 days, or once per days for at least 14 days.

In another sixteenth general embodiment, the present invention includes a method for treating a bacterial infection in a human subject, the method comprising administering an effective amount of an aminoglycoside to the subject not more than once per day for not more than five days. In various embodiments, the aminoglycoside is administered to the subject for 1 to 5 days, 2 to 5 days, 3 to 5 days, or 4 to 5 days.

In one first general embodiment of the second aspect of the invention, the present invention provides an aminoglycoside for use, or prepared for use, in the treatment of a bacterial infection in a human subject by administration of an effective amount of the aminoglycoside to the subject not more than once per day for not more than five days, the effective amount being a potency-normalized amount of at least N_{GEN}×7 mg/kg/day, where N_{GEN}=MIC_{AG}/MIC_{GEN} is a normalizing factor defined by the ratio of a minimum inhibitory concentration of the administered aminoglycoside, MIC_{AG}, to a minimum inhibitory concentration of gentamicin, MIC_{GEN}. In certain embodiments, the effective amount is a potency-normalized amount of at least N_{GEN}×9 mg/kg/day. The minimum inhibitory concentration (e.g., a mininmum inhibitory concentration (90%)) of the administered aminoglycoside and gentamicin can be for the bacteria type (e.g., species or strain) infecting the subject.

In a second general embodiment of the second aspect of the invention, the present invention provides an aminoglycoside for use, or prepared for use, in the treatment of a bacterial infection in a human subject by administration of an effective amount of the aminoglycoside to the subject not more than once per day for not more than five days, the effective amount being a toxicity-normalized amount of equal to or less than T_{GEN}×50 mg/kg/day, where T_{GEN}=MTC_{AG}/MTC_{GEN} is a normalizing factor defined by the ratio of a minimum toxic concentration of the administered aminoglycoside, MTC_{AG}, to a minimum toxic concentration of gentamicin, MTC_{GEN}. In certain embodiments, the effective amount may be in the range of T_{GEN}×30 mg/kg/day to T_{GEN}×50 mg/kg/day or from T_{GEN}×15 mg/kg/day to T_{GEN}×50 mg/kg/day.

In another third general embodiment, related to the first and second embodiments of the second aspect of the invention, the present invention provides an aminoglycoside for use, or prepared for use, in the treatment of a bacterial infection in a human subject by administration of an effective amount of the aminoglycoside to the subject not more than once per day for not more than five days, the effective amount being a potency-normalized amount of at least N_{GEN}×9 mg/kg/day, where N_{GEN}=MIC_{AG}/MIC_{GEN} is a normalizing factor defined by the ratio of a minimum inhibitory concentration of the administered aminoglycoside, MIC_{AG}, to a minimum inhibitory concentration of gentamicin, MIC_{GEN}, and the effective amount being a toxicity-normalized amount of equal to or less than T_{GEN}×50 mg/kg/day, where T_{GEN}=MTC_{AG}/MTC_{GEN} is a normalizing factor defined by the ratio of a minimum toxic concentration of the administered aminoglycoside, MTC_{AG}, to a minimum toxic concentration of gentamicin, MTC_{GEN}.

In another fourth general embodiment of the second aspect of the invention, the present invention provides an aminoglycoside for use, or prepared for use, in the treatment of a bacterial infection in a human subject by administration of an effective amount of the aminoglycoside to the subject not more than once per day for not more than five days to achieve a maximum serum concentration of the administered aminoglycoside, Cₘₐₓ, equal to at least 5 times the minimum inhibitory concentration of the administered aminoglycoside, MIC_{AG}, for the bacteria type (i.e., species or strain) infecting the subject. In certain embodiments, Cₘₐₓ is equal to at least 8 times the minimum inhibitory concentration of the administered aminoglycoside, MIC_{AG}

In a further fifth general embodiment of the second aspect of the invention, the present invention provides an aminoglycoside for use, or prepared for use, in the treatment of a bacterial infection in a human subject by administration of an effective amount of the aminoglycoside to the subject not more than once per day for not more than five days to achieve a maximum serum concentration of the administered aminoglycoside, Cₘₐₓ, and a pharmacokinetic profile defined by a time-concentration curve, the ratio of Cₘₐₓ, to total area under the time-concentration curve, AUC, being at least 0.4 hr⁻¹.

In a further sixth general embodiment of the second aspect of the invention, the present invention provides an aminoglycoside for use, or prepared for use, in the treatment of a bacterial infection in a human subject by administration of an effective amount of the aminoglycoside to the subject not more than once per day for not more than five days to achieve a serum pharmacokinetic profile defined by a time-concentration curve, at least 30% of total area under the time-concentration curve, AUC, being an area above a kidney saturation concentration, C_{KS}, for the aminoglycoside.

In a further seventh general embodiment of the second aspect of the invention, the present invention provides an aminoglycoside for use, or prepared for use, in the treatment of a bacterial infection in a human subject by administration of an effective amount of the aminoglycoside to the subject not more than once per day for at least two days by intravenous infusion over an infusion period of less than or equal to about 15 minutes. In particular embodiments, the aminoglycoside is administered over an infusion period of not more than about 10 minutes.

In another eighth general embodiment related to the seventh general embodiment of second aspect of the invention, the present invention provides an aminoglycoside for use, or prepared for use, in the treatment of a bacterial infection in a human subject by administration of an effective amount of the aminoglycoside to the subject not more than once per day for at least two days by intravenous infusion with an infusion rate of at least N_{GEN}×0.3 mg/kg/min, where N_{GEN}=MIC_{AG}/MIC_{GEN} is a normalizing factor defined by the ratio of a minimum inhibitory concentration of the administered aminoglycoside, MIC_{AG}, to a minimum inhibitory concentration of gentamicin, MIC_{GEN}. In particular embodiments, the infusion rate is at least N_{GEN}×0.5 mg/kg/min. The minimum inhibitory concentration (e.g., a mininmum inhibitory concentration (90%)) of the administered aminoglycoside and gentamicin can be for the bacteria type (e.g., species or strain) infecting the subject.

In a further ninth general embodiment of the second aspect of the invention, the present invention provides an aminoglycoside for use, or prepared for use, in the treatment of a bacterial infection in a human subject by administration of an effective amount of the aminoglycoside to the subject by intraveneous infusion, the infused amount being a potency-normalized amount of at least N_{GEN}×12 mg/kg/infusion, where N_{GEN}=MIC_{AG}/MIC_{GEN} is a normalizing factor defined by the ratio of a minimum inhibitory concentration of the administered aminoglycoside, MIC_{AG}, to a minimum inhibitory concentration of gentamicin, MIC_{GEN}. In certain embodiments, the infused amount is a potency-normalized amount of at least N_{GEN}×15 mg/kg/infusion. The minimum inhibitory concentration (e.g., a mininmum inhibitory concentration (90%)) of the administered aminoglycoside and gentamicin can be for the bacteria type (e.g., species or strain) infecting the subject.

In another tenth general embodiment, related to the fourth and seventh through ninth general embodiments of the second aspect of the invention, the present invention provides an aminoglycoside for use, or prepared for use, in the treatment of a bacterial infection in a human subject by administration of an effective amount of the aminoglycoside to the subject by intravenous infusion to achieve a maximum serum concentration of the administered aminoglycoside, Cₘₐₓ, equal to at least 5 times the minimum inhibitory concentration of the administered aminoglycoside, MIC_{AG}, for the bacteria type infecting the subject. In particular embodiments, the Cₘₐₓ is equal to at least 8 times the minimum inhibitory concentration of the administered aminoglycoside, MIC_{AG}, for the bacteria type (i.e., species or strain) infecting the subject.

In another eleventh general embodiment, related to the fifth and seventh through ninth general embodiments of the second aspect of the invention, the present invention provides an aminoglycoside for use, or prepared for use, in the treatment of a bacterial infection in a human subject by administration of an effective amount of the aminoglycoside to the subject by intravenous infusion to achieve a maximum serum concentration of the administered aminoglycoside, Cₘₐₓ, and a pharmacokinetic profile defined by a time-concentration curve, the ratio of Cₘₐₓ, to total area under the time-concentration curve, AUC, being at least 0.4 hr⁻¹. In particular embodiments, the ratio of Cₘₐₓ to total area under the time-concentration curve, AUC is at least 0.6 hr⁻¹.

In an additional twelvth general embodiment, related to the sixth through ninth general embodiments of the second aspect of the invention, the present invention provides an aminoglycoside for use, or prepared for use, in the treatment of a bacterial infection in a human subject by administration of an effective amount of the aminoglycoside to the subject by intravenous infusion to achieve a serum pharmacokinetic profile defined by a time-concentration curve, at least 30% of total area under the time-concentration curve, AUC, being an area above a kidney saturation concentration, C_{KS}, for the aminoglycoside. This general embodiment can alternatively, additionally or more particularly include other features as described herein, including as described in connection with the sixth through ninth general emodiments of the second aspect of the invention.

In another thirteenth general embodiment of the second aspect of the invention, the present invention provides an aminoglycoside for use, or prepared for use, in the treatment of a bacterial infection in a human subject by administration of an effective amount of the aminoglycoside to the subject not more than once per day for not more than five days, and maintaining substantially baseline renal function as indicated by one or more nephrotoxicity biomarkers.

In a further fourteenth general embodiment of the second aspect of the invention, the present invention provides an aminoglycoside for use, or prepared for use, in the treatment of a bacterial infection in a subject without causing otoxicity, or alternatively, while substantially maintaining baseline auditory response, by administration of an effective amount of the aminoglycoside to the subject to achieve a maximum serum concentration of the administered aminoglycoside, Cₘₐₓ, equal to at least 8 times the minimum inhibitory concentration of the administered aminoglycoside, MIC_{AG}, for the bacteria type (i.e., species or strain) infecting the subject, wherein the aminoglycoside is administered once per day for at least five days.

In a fifteenth general embodiment, related to the fourteenth general embodiment of the second aspect of the invention, the present invention provides an aminoglycoside for use, or prepared for use, in the treatment of a bacterial infection in a human subject, by administration of an effective amount of the aminoglycoside to the subject to achieve a maximum serum concentration of the administered aminoglycoside, Cₘₐₓ, equal to at least 8 times the minimum inhibitory concentration (e.g., a minumum inhibitory concentration (90%)) of the administered aminoglycoside, MIC_{AG}, for the bacterial type (i.e., species or strain) infecting the subject at least once per day, and maintaining substantially baseline auditory function as indicated by one or more auditory markers. The auditory markers can be an auditory brainstem response (ABR).

In another sixteenth general embodiment of the second aspect of the invention, the present invention provides an aminoglycoside for use, or prepared for use, in the treatment of a bacterial infection in a human subject by administration of an effective amount of the aminoglycoside to the subject not more than once per day for not more than five days.

In a first general embodiment of the third aspect of the invention, the present invention provides for the use of an aminoglycoside for the manufacture of a medicament for the treatment of, or prepared for the treatment of, a bacterial infection in a human subject, wherein the medicament is administered in an effective amount not more than once per day for not more than five days, the effective amount being a potency-normalized amount of at least N_{GEN}×7 mg/kg/day, where N_{GEN}=MIC_{AG}/MIC_{GEN} is a normalizing factor defined by the ratio of a minimum inhibitory concentration of the administered aminoglycoside, MIC_{AG}, to a minimum inhibitory concentration of gentamicin, MIC_{GEN}. In certain embodiments, the effective amount is a potency-normalized amount of at least N_{GEN}×9 mg/kg/day. The minimum inhibitory concentration (e.g., a mininmum inhibitory concentration (90%)) of the administered aminoglycoside and gentamicin can be for the bacteria type (e.g., species or strain) infecting the subject.

In a further second general embodiment of the third aspect of the invention, the present invention provides for the use of an aminoglycoside for the manufacture of a medicament for the treatment of, or prepared for the treatment of, a bacterial infection in a human subject, wherein the medicament is administered in an effective amount not more than once per day for not more than five days, the effective amount being a toxicity-normalized amount of equal to or less than T_{GEN}×50 mg/kg/day, where T_{GEN}=MTC_{AG}/MTC_{GEN} is a normalizing factor defined by the ratio of a minimum toxic concentration of the administered aminoglycoside, MTC_{AG}, to a minimum toxic concentration of gentamicin, MTC_{GEN}. In certain embodiments, the effective amount may be in the range of T_{GEN}×30 mg/kg/day to T_{GEN}×50 mg/kg/day or from T_{GEN}×15 mg/kg/day to T_{GEN}×50 mg/kg/day.

In a third general embodiment, related to the first and second general embodiments of the third aspect of the invention, the present invention provides for the use of an aminoglycoside for the manufacture of a medicament for the treatment of, or prepared for the treatment of, a bacterial infection in a human subject, wherein the medicament is administered in an effective amount not more than once per day for not more than five days, the effective amount being a potency-normalized amount of at least N_{GEN}×9 mg/kg/day, where N_{GEN}=MIC_{AG}/MIC_{GEN} is a normalizing factor defined by the ratio of a minimum inhibitory concentration of the administered aminoglycoside, MIC_{AG}, to a minimum inhibitory concentration of gentamicin, MIC_{GEN}, and the effective amount being a toxicity-normalized amount of equal to or less than T_{GEN}×50 mg/kg/day, where T_{GEN}=MTC_{AG}/MTC_{GEN} is a normalizing factor defined by the ratio of a minimum toxic concentration of the administered aminoglycoside, MTC_{AG}, to a minimum toxic concentration of gentamicin, MTC_{GEN}.

In another fourth general embodiment of the third aspect of the invention, the present invention provides for the use of an aminoglycoside for the manufacture of a medicament for the treatment of, or prepared for the treatment of, a bacterial infection in a human subject, wherein the medicament is administered in an effective amount not more than once per day for not more than five days to achieve a maximum serum concentration of the administered aminoglycoside, Cₘₐₓ, equal to at least 5 times the minimum inhibitory concentration of the administered aminoglycoside, MIC_{AG}, for the bacteria type (i.e., species or strain) infecting the subject. In certain embodiments, the Cₘₐₓ is equal to at least 8 times the minimum inhibitory concentration of the administered aminoglycoside, MIC_{AG}.

In a further fifth general embodiment of the third aspect of the invention, the present invention provides for the use of an aminoglycoside for the manufacture of a medicament for the for the treatment of, or prepared for the treatment of, a bacterial infection in a human subject, wherein the medicament is administered not more than once per day for not more than five days to achieve a maximum serum concentration of the administered aminoglycoside, Cₘₐₓ, and a pharmacokinetic profile defined by a time-concentration curve, the ratio of Cₘₐₓ to total area under the time-concentration curve, AUC, being at least 0.4 hr⁻¹.

In another sixth general embodiment of the third aspect of the invention, the present invention provides for the use of an aminoglycoside for the manufacture of a medicament for the treatment of, or prepared for the treatment of, a bacterial infection in a human subject, wherein the medicament is administered in an effective amount to the subject not more than once per day for not more than five days to achieve a serum pharmacokinetic profile defined by a time-concentration curve, at least 30% of total area under the time-concentration curve, AUC, being an area above a kidney saturation concentration, C_{KS}, for the aminoglycoside.

In a further seventh general embodiment of the third aspect of the invention, the present invention provides for the use of an aminoglycoside for the manufacture of a medicament for the treatment of, or prepared for the treatment of, a bacterial infection in a human subject, wherein the medicament is administered in an effective amount not more than once per day for at least two days by intravenous infusion over an infusion period of less than or equal to about 15 minutes. In particular embodiments, the aminoglycoside is administered over an infusion period of not more than about 10 minutes.

In another eighth general embodiment, related to the seventh general embodiment of the third aspect of the invention, the present invention provides for the use of an aminoglycoside for the manufacture of a medicament for the treatment of, or prepared for the treatment of, a bacterial infection in a human subject, wherein the medicament is administered in an effective amount not more than once per day for at least two days by intravenous infusion with an infusion rate of at least N_{GEN}×0.3 mg/kg/min, where N_{GEN}=MIC_{AG}/MIC_{GEN} is a normalizing factor defined by the ratio of a minimum inhibitory concentration of the administered aminoglycoside, MIC_{AG}, to a minimum inhibitory concentration of gentamicin, MIC_{GEN}. In particular embodiments, the infusion rate is at least N_{GEN}×0.5 mg/kg/min. The minimum inhibitory concentration (e.g., a mininmum inhibitory concentration (90%)) of the administered aminoglycoside and gentamicin can be for the bacteria type (e.g., species or strain) infecting the subject.

In another ninth general embodiment of the third aspect of the invention, the present invention provides for the use of an aminoglycoside for the manufacture of a medicament for the treatment of, or prepared for the treatment of, a bacterial infection in a human subject, wherein the medicament is administered in an effective amount by intraveneous infusion, the infused amount being a potency-normalized amount of at least N_{GEN}×12 mg/kg/infusion, where N_{GEN}=MIC_{AG}/MIC_{GEN} is a normalizing factor defined by the ratio of a minimum inhibitory concentration of the administered aminoglycoside, MIC_{AG}, to a minimum inhibitory concentration of gentamicin, MIC_{GEN}. In certain embodiments, the infused amount is a potency-normalized amount of at least N_{GEN}×15 mg/kg/infusion. The minimum inhibitory concentration (e.g., a mininmum inhibitory concentration (90%)) of the administered aminoglycoside and gentamicin can be for the bacteria type (e.g., species or strain) infecting the subject.

In another tenth general embodiment, related to the fourth and seventh through ninth general embodiments of the third aspect of the invention, the present invention provides for the use of an aminoglycoside for the manufacture of a medicament for the treatment of, or prepared for the treatment of, a bacterial infection in a human subject, wherein the medicament is administered in an effective amount by intravenous infusion to achieve a maximum serum concentration of the administered aminoglycoside, Cₘₐₓ, equal to at least 5 times the minimum inhibitory concentration of the administered aminoglycoside, MIC_{AG}, for the bacteria type (i.e., species or strain) infecting the subject. In particular embodiments, the Cₘₐₓ is equal to at least 8 times the minimum inhibitory concentration of the administered aminoglycoside, MIC_{AG}, for the bacteria type infecting the subject.

In an eleventh general embodiment, related to the fifth and seventh through ninth general embodiments of the third aspect of the invention, the present invention provides for the use of an aminoglycoside for the manufacture of a medicament for the treatment of, or prepared for the treatment of, a bacterial infection in a human subject, wherein the medicament is administered in an effective amount by intravenous infusion to achieve a maximum serum concentration of the administered aminoglycoside, Cₘₐₓ, and a pharmacokinetic profile defined by a time-concentration curve, the ratio of Cₘₐₓ to total area under the time-concentration curve, AUC, being at least 0.4 hr⁻¹. In particular embodiments, the ratio of Cₘₐₓ to total area under the time-concentration curve, AUC is at least 0.6 hr⁻¹.

In a further twelvth general embodiment, related to the sixth through ninth general embodiments of the third aspect of the invention, the present invention provides for the use of an aminoglycoside for the manufacture of a medicament for the treatment of, or prepared for the treatment of, a bacterial infection in a human subject, wherein the medicament is administered in an effective amount by intravenous infusion to achieve a serum pharmacokinetic profile defined by a time-concentration curve, at least 30% of total area under the time-concentration curve, AUC, being an area above a kidney saturation concentration, C_{KS}, for the aminoglycoside. This general embodiment can alternatively, additionally or more particularly include other features as described herein, including as described in connection with the sixth through ninth general emodiments of the third aspect of the invention.

In another thirteenth general embodiment of the third aspect of the invention, the present invention provides for the use of an aminoglycoside for the manufacture of a medicament for the treatment of, or prepared for the treatment of, a bacterial infection in a human subject, wherein the medicament is administered in an effective amount not more than once per day for not more than five days, and maintaining substantially baseline renal function as indicated by one or more nephrotoxicity biomarkers.

In another fourteenth embodiment of the third aspect of the invention, the present invention provides for the use of an aminoglycoside for the manufacture of a medicament for the treatment of, or prepared for the treatment of, a bacterial infection in a human subject without causing otoxicity, or alternatively, while substantially maintaining baseline auditory response, wherein the medicament is administered in an effective amount to achieve a maximum serum concentration of the administered aminoglycoside, Cₘₐₓ, equal to at least 8 times the minimum inhibitory concentration of the administered aminoglycoside, MIC_{AG}, for the bacteria type (i.e., species or strain) infecting the subject, wherein the aminoglycoside is administered once per day for at least five days.

In a fifteenth general embodiment, related to the fourteenth general embodiment of the third aspect of the invention, the present invention provides for the use of an aminoglycoside for the manufacture of a medicament for the treatment of, or prepared for the treatment of, a bacterial infection in a human subject, wherein the medicament is administered in an effective amount to achieve a maximum serum concentration of the administered aminoglycoside, Cₘₐₓ, equal to at least 8 times the minimum inhibitory concentration (e.g., a minumum inhibitory concentration (90%)) of the administered aminoglycoside, MIC_{AG}, for the bacterial type (i.e., species or strain) infecting the subject at least once per day, and maintaining substantially baseline auditory function as indicated by one or more auditory markers. The auditory markers can be an auditory brainstem response (ABR).

In another sixteenth general embodiment of the third aspect of the invention, the present invention provides for the use of an aminoglycoside for the manufacture of a medicament for the treatment of, or prepared for the treatment of, a bacterial infection in a human subject, wherein the medicament is administered in an effective amount not more than once per day for not more than five days.

Any of the more specific features described herein with respect to the first through sixteenth general embodiments of the first aspect of the invention may also be included within the first through sixteenth general embodiments of the second and third aspect of the invention, respectively. Reference to various embodiments below is intended to include those embodiments of any aspect of the invention.

Additionally in connection with any of the fourth through sixteenth general embodiments, the effective amount administered to a subject can be a potency-normalized amount of at least N_{GEN}×7 mg/kg/day, at least N_{GEN}×8 mg/kg/day, or at least N_{GEN}×9 mg/kg/day, where N_{GEN}=MIC_{AG}/MIC_{GEN} is a normalizing factor defined by the ratio of a minimum inhibitory concentration (e.g., a minumum inhibitory concentration (90%)) of the administered aminoglycoside, MIC_{AG}, to a minimum inhibitory concentration (e.g., a minumum inhibitory concentration (90%)) of gentamicin, MIC_{GEN}. Additionally or alternatively, the effective amount can be equal to or less than a toxicity-normalized amount of T_{GEN}×50 mg/kg/day, where T_{GEN}=MTC_{AG}/MTC_{GEN} is a normalizing factor defined by the ratio of a minimum toxic concentration of the administered aminoglycoside, MTC_{AG}, to a minimum toxic concentration of gentamicin, MTC_{GEN}. The effective amount can be at least a potentcy-normalized amount and/or equal to or less than a toxicity-normalized amount as described herein, including as described in connection with the first through third general embodiments of the first aspect, considered alone or in various combinations and permutations.

Additionally in connection with any of the first through third and sixth through sixteenth general embodiments, the effective amount of an aminoglycoside can be administered to the subject to achieve a pharmacokinetic profile (e.g., a serum pharmacokinetic profile) as described herein. The effective amount of an aminoglycoside can be administered to the subject to achieve a maximum serum concentration of the administered aminoglycoside, Cₘₐₓ, equal to at least 5 times the minimum inhibitory concentration (e.g., a minumum inhibitory concentration (90%)) of the administered aminoglycoside, MIC_{AG}, for the bacterial type (i.e., species or strain) infecting the subject. Additionally or alternatively, the effective amount of an aminoglycoside can be administered to the subject to achieve a maximum serum concentration of the administered aminoglycoside, Cₘₐₓ, and a pharmacokinetic profile (e.g., a serum pharmacokinetic profile) defined by a time-concentration curve, the ratio of Cₘₐₓ to total area under the time-concentration curve, AUC, being at least 0.4 hr Additionally or alternatively, the effective amount of an aminoglycoside can be administered to the subject to achieve a pharmacokinetic profile (e.g., a serum pharmacokinetic profile) defined by a time-concentration curve, at least 30% of total area under the time-concentration curve, AUC, being an area above a kidney saturation concentration (C_{KS}). Such administration can be as described herein, including as described in connection with the fourth through sixth general embodiments of the first aspect, considered alone or in various combinations and permutations.

Additionally in connection with any of the first through sixth and the thirteenth through sixteenth general embodiments, the effective amount of an aminoglycoside can be administered to the subject by intravenous infusion. The effective amount of an aminoglycoside can be administered to the subject not more than once per day for at least two days by intravenous infusion over an infusion period of less than or equal to about 60 minutes, or 30 minutes, or 15 minutes. Additionally or alternatively, the effective amount of an aminoglycoside can be administered to the subject not more than once per day for at least two days by intravenous infusion with an infusion rate of at least N_{GEN}×0.3 mg/kg/min, where N_{GEN}=MIC_{AG}/MIC_{GEN} is a normalizing factor defined by the ratio of a minimum inhibitory concentration (e.g., a minumum inhibitory concentration (90%)) of the administered aminoglycoside, MIC_{AG}, to a minimum inhibitory concentration (e.g., a minumum inhibitory concentration (90%)) of gentamicin, MIC_{GEN}, for the bacterial type (i.e., species or strain) infecting the subject. Additionally or alternatively, the infused amount can be a potency-normalized amount of at least N_{GEN}×12 mg/kg/infusion, where N_{GEN}=MIC_{AG}/MIC_{GEN} is a normalizing factor defined by the ratio of a minimum inhibitory concentration (e.g., a minumum inhibitory concentration (90%)) of the administered aminoglycoside, MIC_{AG}, to a minimum inhibitory concentration (e.g., a minumum inhibitory concentration (90%)) of gentamicin, MIC_{GEN}, for the bacterial type (i.e., species or strain) infecting the subject. Additionally or alternatively, the effective amount can be administered by intravenous infusion to achieve a maximum serum concentration of the administered aminoglycoside, Cₘₐₓ, equal to at least 5 times the minimum inhibitory concentration (e.g., a minumum inhibitory concentration (90%)) of the administered aminoglycoside, MIC_{AG}, for the bacterial type (i.e., species or strain) infecting the subject. Additionally or alternatively, the effective amount can be administered by intravenous infusion to achieve a maximum serum concentration of the administered aminoglycoside, Cₘₐₓ, and a pharmacokinetic profile (e.g., a serum pharmacokinetic profile) defined by a time-concentration curve, the ratio of Cₘₐₓ to total area under the time-concentration curve, AUC, being at least 0.4 hr⁻¹. Additionally or alternatively, the effective amount can be administered by intravenous infusion to achieve a serum pharmacokinetic profile defined by a time-concentration curve, at least 30% of total area under the time-concentration curve, AUC, being an area above a kidney saturation concentration, C_{KS}, for the aminoglycoside. The administration by intraveneous infusion can be as described herein, including as described in connection with the seventh through twelvth general embodiments of the first aspect, considered alone or in various combinations and permutations.

Additionally in connection with any of the first through twelvth and the sixteenth general embodiments, the effective amount of an aminoglycoside can be administered to the subject without causing clinically relevant nephrotoxicity and/or without causing clinically relevant ototoxicity. The effective amount can be administered to the subject while maintaining substantially baseline renal function as indicated by one or more nephrotoxicity biomarkers. Additinoally or alternatively, the effective amount of an aminoglycoside can be adminstered to the subject while maintaining substantially baseline auditory function as indicated by one or more auditory markers. Such administration can be as described herein, including as described in connection with the thirtheenth through fifteenth general embodiments of the first aspect, considered alone or in various combinations and permutations.

In various embodients, including as relevant any of the first through fifteenth general embodiments or subembodiments thereof as described herein, N_{GEN} may be defined by the ratio of a minimum inhibitory concentration (e.g., a minumum inhibitory concentration (90%)) of the administered aminoglycoside, MIC_{AG}, to a minimum inhibitory concentration (e.g., a minumum inhibitory concentration (90%)) of gentamicin, MIC_{GEN}, for a bacterial strain selected from the E. coli ATCC strain 25922, the P. aeruginosa ATCC strain 27853, or the S. aureus ATCC strain 29213.

In various embodiments, including as relevant any of the first through fifteenth general embodiments or subembodiments thereof as described herein, the aminoglycoside cam be administered to the subject for more than 5 days, more than 7 days, more than 10 days, or more than 14 days.

In particular embodiments, including as relevant any of the first through fifteenth general embodiments or subembodiments thereof as described herein, the aminoglycoside is 6′-(2-Hydroxy-ethyl)-1-(4-amino-2(S)-hydroxy-butyryl)-sisomicin, or a steroisomer, pharmaceutically acceptable salt, or prodrug thereof.

It is understood that the embodiments described herein, including any of the first through fifteenth general embodiments or any subembodiments thereof as described herein, including such as those referred to as “one embodiment” may include additional features, structures or characterics, such as the features, structures, or characteriscs described herein with respect to “particular” or “further” embodiments. Accordingly, any of the particular features, structures, or characteristics described herein may be present in any of the various embodiments of the present invention, and the particular features, structures, or characteristics may be combined in any suitable manner in one or more embodiments.

In particular embodiments of any of the methods of the present invention, the aminoglycoside is administered to the subject for not more than five days, not more than four days, not more than three days, or not more than once per day.

In particular embodiments of any of the methods of the present invention, the total daily amount of the aminoglycoside administered to the subject is a potency-normalized amount of at least N_{GEN}×7 mg/kg/day, at least N_{GEN}×9 mg/kg/day, at least N_{GEN}×12 mg/kg/day, at least N_{GEN}×15 mg/kg/day, at least N_{GEN}×20 mg/kg/day, or at least N_{GEN}×25 mg/kg/day.

In particular embodiments of any of the methods of the present invention, the aminoglycoside is administered to the subject by intraveneous infusion.

In particular embodiments of any of the methods of the present invention, the aminoglycoside is administered in an amount effective for treating a bacterial infection as determined by clinical response or eradication of the bacteria from the site of infection in the subject.

In particular embodiments of any of the methods of the present invention, the aminoglycoside is administered in an effective amount to achieve a maximum serum concentration of the administered aminoglycoside, Cₘₐₓ, equal to at least 8 times the minimum inhibitory concentration of the administered aminoglycoside, MIC_{AG}, for the bacterial type (i.e., species or strain) infecting the subject. In one embodiment, the ratio of Cₘₐₓ to MIC_{AG} ranges from about 8 to about 96.

In particular embodiments of any of the methods of the present invention, the aminoglycoside is administered in an effective amount to achieve a maximum serum concentration of the administered aminoglycoside, Cₘₐₓ, and a pharmacokinetic profile defined by a time-concentration curve, the ratio of Cₘₐₓ, to total area under the time-concentration curve, AUC, being at least 0.6 hr⁻¹. In one embodiment, the ratio of Cₘₐₓ to AUC ranges from 0.6 to 1.0 hr⁻¹. In another embodiment, the aminoglycoside is administered in an effective amount to achieve a maximum serum concentration of the administered aminoglycoside, Cₘₐₓ, and a pharmacokinetic profile defined by a time-concentration curve, the ratio of Cₘₐₓ, to total area under the time-concentration curve, AUC, being at least 0.4 hr⁻¹. In one embodiment, the ratio of Cₘₐₓ to AUC ranges from 0.4 to 1.0 hr⁻¹.

In particular embodiments of any of the methods of the present invention, the aminoglycoside is administered in an effective amount to achieve a serum pharmacokinetic profile defined by a time-concentration curve, at least 30% of total area under the time-concentration curve, AUC, being an area above a kidney saturation concentration, C_{KS}, for the aminoglycoside. In one embodiment, at least 50% of the AUC is above the kidney saturation concentration, C_{KS}, for the aminoglycoside.

In particular embodiments of any of the methods of the present invention, the aminoglycoside is administered in an amount effective for treating an infection of a Gram negative bacteria, a Gram positive bacteria, an Enterobacteriaceae bacteria, a K. pneumoniae bacteria, an E. coli bacteria, an Enterobacter bacteria, or a Staphylococcus aureus bacterium.

In particular embodiments of any of the methods of the present invention, the aminoglycoside is administered in an amount effective for treating an infection of a bacterial strain having resistance to at least one antibacterial agent. In various embodiments, the bacterial strain comprises an aminoglycoside-resistance mechanism, expresses an aminoglycoside-modifying enzyme (AME) associated with aminoglycoside resistance, expresses one or more of a β-lactamase, metallo-β-lactamase, Klebsiella pneumoniae carbapenemase or other carbapenemase, a DNA gyrase (e.g., a mutated DNA gyrase), an aminoglycoside resistance methylase (e.g., ArmA), and/or is resistant to gentamicin, tobramycin, or amikacin. In particular embodiments, the bacterial strain is a S. aureus strain resistant to methicillin (MRSA), a S. aureus strain resistant to vancomycin (VRSA), or a coagulase-negative staphylococci.

In particular embodiments of any of the methods of the present invention, the aminoglycoside has broad spectrum gram negative antibacterial activity. In particular embodiment, the aminoglycoside has antibacterial activity against one or more of the following bacteria: E. coli, Klebsiella spp., Enterobacter spp., Citrobacter spp., P. mirabilis, M. morganii, P. aeruginosa, S. aureus, S. saprophyticus.

In certain embodiments, the administered aminoglycoside is amikacin, gentamycin, tobramycin, netromycin, apramycin, streptomycin, kanamycin, dibekacin, arbekacin, paromomycin, neomycin, netilmicin, or sisomicin, or an analog, stereoisomer, pharmaceutically acceptable salt or prodrug thereof. In related embodiments, the administered aminoglycoside is sisomycin, amikacin, kanamycin, arbekacin, dibekacin, tobramycin, neomycin, or gentamicin, or an analog, stereoisomer, pharmaceutically acceptable salt or prodrug thereof. In particular embodiments, the aminoglycoside is sisomicin, gentamicin, amikacin, or neomycin, or an analog, stereoisomer, pharmaceutically acceptable salt or prodrug thereof. In certain embodiments, the aminoglycoside is sisomicin, or an analog, stereoisomer, pharmaceutically acceptable salt or prodrug thereof. In certain embodiments, the aminoglycoside is gentamicin, or an analog, stereoisomer, pharmaceutically acceptable salt or prodrug thereof. In certain embodiments, the aminoglycoside is amikacin, or an analog, stereoisomer, pharmaceutically acceptable salt or prodrug thereof. In certain embodiments, the aminoglycoside is neomycin, or an analog, stereoisomer, pharmaceutically acceptable salt or prodrug thereof.

In particular embodiments, the aminoglycoside is a sisomicin analog having structure (A), or a stereoisomer, pharmaceutically acceptable salt or prodrug thereof. In other particular embodiments, the aminoglycoside is a sisomicin analog having structure (I) or (II), or a stereoisomer, pharmaceutically acceptable salt or prodrug thereof. In certain embodiments, the aminoglycoside is 6′-(2-Hydroxy-ethyl)-1-(4-amino-2(S)-hydroxy-butyryl)-sisomicin, or a stereoisomer, pharmaceutically acceptable salt or prodrug thereof.

In particular embodiments, the aminoglycoside is a kanamycin analog having structure (B), or a stereoisomer, pharmaceutically acceptable salt or prodrug thereof. In other particular embodiments, the aminoglycoside is a kanamycin analog having structure (III) or structure (IV), or a stereoisomer, pharmaceutically acceptable salt or prodrug thereof.

In particular embodiments, the aminoglycoside is a dibekacin analog having structure (C), or a stereoisomer, pharmaceutically acceptable salt or prodrug thereof. In other particular embodiments, the aminoglycoside is a dibekacin analog having structure (V), or a stereoisomer, pharmaceutically acceptable salt or prodrug thereof.

In particular embodiments, the aminoglycoside is a tobramycin analog having structure (D), or a stereoisomer, pharmaceutically acceptable salt or prodrug thereof. In other particular embodiments, the aminoglycoside is a tobramycin analog having structure (VI), or a stereoisomer, pharmaceutically acceptable salt or prodrug thereof.

In particular embodiments, the aminoglycoside is a gentamicin analog having structure (E), or a stereoisomer, pharmaceutically acceptable salt or prodrug thereof. In other particular embodiments, the aminoglycoside is a gentamicin analog having structure (VII) or structure (VIII), or a stereoisomer, pharmaceutically acceptable salt or prodrug thereof.

In particular embodiments, the aminoglycoside is a neomycin analog having structure (F), or a stereoisomer, pharmaceutically acceptable salt or prodrug thereof.

In a related embodiment, the present invention includes a method for treating a bacterial infection in a human subject, the method comprising administering an effective amount of the compound, 6′-(2-Hydroxy-ethyl)-1-(4-amino-2(S)-hydroxy-butyryl)-sisomicin, or a stereoisomer, pharmaceutically acceptable salt or prodrug thereof, to the subject, wherein said compound is administered at a dosage of at least 10 mg/kg subject body weight not more than once per day for not more than five days.

In a further related embodiment, the present invention includes the compound, 6′-(2-Hydroxy-ethyl)-1-(4-amino-2(S)-hydroxy-butyryl)-sisomicin, or a stereoisomer, pharmaceutically acceptable salt or prodrug thereof, for use, or prepared for use, in the treatment of a bacterial infection in a human subject, by administration to the subject of a dosage of at least 10 mg/kg subject body weight not more than once per day for not more than five days.

In another related embodiment, the present invention provides for the use of an aminoglycoside for the manufacture of a medicament for the treatment of, or prepared for the treatment of, a bacterial infection in a human subject, wherein the medicament is administered to the subject at a dosage of at least 10 mg/kg subject body weight not more than once per day for not more than five days.

In certain embodiments, the bacterial infection in the subject is a urinary tract infection (UTI). In one embodiment the UTI is a complicated UTI (cUTI). In another embodiment, the UTI is an uncomplicated UTI (uUTI).

In particular embodiments, the compound is administered at a dosage of at least 15 mg/kg subject body weight, at least 20 mg/kg subject body weight, at least 25 mg/kg body weight, or at least 30 mg/kg body weight. In one embodiment, the compound is administered at a dosage in the range of 15 mg/kg subject body weight to 25 mg/kg subject body weight or in the range of 15 mg/kg subject body weight to 30 mg/kg subject body weight. In particular embodiments, the compound is administered at a dosage of about 15 mg/kg subject body weight

In certain embodiments, the compound is administered for not more than four days or not more than three days.

In one embodiment, said compound is administered by intravenous infusion. In certain embodiments, the infusion occurs over a time period between about 10 minutes and about 15 minutes, over a time period less than or equal to 15 minutes, or over a time period less than or equal to 10 minutes. In additional embodiments, the compound is administered by intravenous infusion over a time period between about 5 minutes and 30 minutes, or between about 5 minutes and 15 minutes.

In particular embodiments, the subject treated with the compound is infected with an Enterobacteriaceae bacteria, E. coli, Pseudomonas aeruginosa, K. peumoniae, Staphylococcus saprophyticus, or Proteus mirabilis. In particular embodiments, the subject is infected with a drug-resistant bacteria, e.g., a multi-drug-resistant bacteria.

In certain embodiments of the invention, the MIC_{AG} and the MIC_{GEN} are determined for the bacteria type infecting the subject. In particular embodiments, the MIC_{AG} and the MIC_{GEN} are determined for the bacterial species, bacterial strain, or bacterial clinical isolate infecting the subject. In certain embodiments, the MIC_{AG} and MIC_{GEN} are determined for a bacterial strain selected from the group consisting of E. coli ATCC strain 25922, P. aeruginosa ATCC strain 27853, and S. aureus ATCC strain 29213. In further embodiments, the MIC_{AG} is a minimum inhibitory concentration (90%) of the administered aminoglycoside (MIC_{AG} (90%)), and the MIC_{GEN} is a minimum inhibitory concentration (90%) of gentamicin (MIC_{GEN} (90%)). In certain embodiments, the MIC_{AG} (90%) and MIC_{GEN} (90%) are determined using a set of strains of a bacterial species. In particular embodiments, the MIC_{AG} (90%) and MIC_{GEN} (90%) are determined using a set of isolates of a bacterial strain. In certain embodiments, the bacterial species or bacterial strain is a bacteria type (i.e., bacterial species or a bacterial strain) infecting the subject. In related embodiments, the MIC_{AG} is a minimum inhibitory concentration (90%) of the administered aminoglycoside (MIC_{AG} (90%)), and the MIC_{GEN} is a minimum inhibitory concentration (90%) of gentamicin (MIC_{GEN} (90%)) for the bacteria type (i.e., species or strain) infecting the subject.

### BRIEF DESCRIPTION OF THE SEVERAL VIEWS OF THE DRAWINGS

FIG. 1 is a graph depicting the mean serum concentration of Compound 1 versus time in mice.

FIG. 2 is a graph depicting the plasma concentrations of Compound 1 versus time in three rats.

FIG. 3 is a graph depicting the plasma concentration of Compound 1 versus time in three dogs. The results for each dog are indicated by the different symbols, i.e., circle, square, or diamond.

FIG. 4 is a graph showing the blood urea nitrogen levels (BUN) of individual rats after 14 days of once-daily dosing of the depicted aminoglycosides at the indicated concentrations. Each filled circle represents an individual measurement.

FIG. 5 is a graph showing the average BUN after 14 days of once-daily dosing of the indicated aminoglycosides.

FIG. 6 is a graph showing the BUN of individual rats after 10 days of the indicated gentamicin dosing, with the equivalent total daily dose given either once-daily, twice-daily, or three times a day. Each filled circle represents an individual measurement.

FIG. 7 is a graph showing the BUN of individual rats after 5 days of once-daily gentamicin dosing at the indicated concentration, with BUN sampled on days 6, 11, and 15. Each filled circle represents an individual measurement.

FIG. 8 is a graph showing the serum creatine of individual rats after 2, 5, 10, or 14 days of once-daily gentamicin dosing at the indicated concentrations. Each filled circle represents an individual measurement.

FIG. 9 is a graph the progression of the indicated kidney histopathology changes after 3, 5, and 14 days of dosing of once-daily gentamicin at the indicated concentrations.

FIGS. 10A-10C are graphs showing the body weight of rats treated with the indicated dosages of gentamicin or tobramycin over a 14 day time course. FIG. 10A shows the results for rats treated with vehicle, gentamicin at 10 mg/kg q.d., gentamicin at 30 mg/kg q.d., gentamicin at 100 mg/kg q.d., or gentamicin at 100 mg/kg b.i.d. for 14 days. FIG. 10B shows the results for rats treated with gentamicin at 10 mg/kg b.i.d., gentamicin at 10 mg/kg t.i.d., gentamicin at 30 mg/kg b.i.d., gentamicin at mg/kg t.i.d., or gentamicin at 100 mg/kg t.i.d. for 14 days. FIG. 10C shows the results for rats treated with gentamicin at 100 mg/kg q.d., 200 mg/kg q.d., or 300 mg/kg q.d., for 5 days, or treated with tobramycin at 10 mg/kg q.d., 30 mg/kg q.d., or 100 mg/kg q.d. for 14 days.

FIGS. 11A and 11B provide a diagram of a design for a phase I dose-escalation study of Compound 1 in healthy volunteers.

FIG. 12 is a logarithmic graph showing the average concentration (±standard deviation) of Compound 1 (Compound) present in the plasma of patients from different Cohorts over 48 hours following administration of a single dose of Compound 1.

FIG. 13 is a logarithmic graph showing the average concentration (±standard deviation) of Compound 1 (Compound) present in the plasma of patients from each of the different Cohorts over 24 hours following administration of a single dose of Compound 1.

FIG. 14 is a linear graph showing the showing the average concentration of Compound 1 (Compound) present in the plasma of patients from each of the different Cohorts over 24 hours following administration of a single dose of Compound 1.

FIG. 15 is a graph showing the concentration of Compound 1 (Compound) in the plasma of a single subject of Cohort 4 over the 48 hours following administration of the single dose (Day=1), administration of the first multi-dose (Day=8), and administration of the third multi-dose (Day=10).

FIGS. 16A and 16B provide graphs showing the plasma Cₘₐₓ vs. dose of Compound 1 (Compound) determined from the various Cohorts (FIG. 16A) and the AUC vs. dose determined from the various Cohorts (FIG. 16B) following administration of Compound 1.

FIG. 17 is a graph showing the average urine concentration of Compound 1 (Compound) in Cohort 4 over the 24 hours following administration of the dose.

FIG. 18 provides graphs depicting the effect of various doses of gentamicin treatment for 14 days on auditory brainstem response (ABR) readouts at each of three hearing frequencies (4, 10, and 20 kHz) in guinea pigs. FIG. 18A shows the results of treatment with vehicle; FIG. 18B shows the results of treatment with 25 mg/kg gentamicin; FIG. 18C shows the results of treatment with 50 mg/kg gentamicin; FIG. 18D shows the results of treatment with 80 mg/kg gentamicin; and FIG. 18E shows the results of treatment with 100 mg/kg of gentamicin. These data demonstrate that gentamicin at 80 or 100 mg/kg/day shows significant threshold shifts (>15 dB).

FIG. 19 provides graphs depicting the effect of various doses of amikacin treatment for 14 days on auditory brainstem response (ABR) readouts at each of three hearing frequencies (4, 10, and 20 kHz) in guinea pigs. FIG. 19A shows the results of treatment with vehicle; FIG. 19B shows the results of treatment with 75 mg/kg amikacin; FIG. 19C shows the results of treatment with 150 mg/kg amikacin; and FIG. 19D shows the results of treatment with 300 mg/kg amikacin. These data demonstrate that amikacin at 300 mg/kg/day shows significant threshold shifts (>15 dB).

FIG. 20 provides graphs depicting the effect of various doses of Compound 1 treatment for 14 days on auditory brainstem response (ABR) readouts at each of three hearing frequencies (4, 10, and 20 kHz) in guinea pigs. FIG. 20A shows the results of treatment with vehicle; FIG. 20B shows the results of treatment with 8 mg/kg Compound 1; FIG. 20C shows the results of treatment with 30 mg/kg Compound 1; and FIG. 20D shows the results of treatment with 80 mg/kg Compound 1. These data demonstrate no significant pre- to post-dosing hearing threshold shift at any frequency. Compound 1 is labeled as “Compound” in this figure.

FIG. 21 provides graphs depicting the effect of duration of gentamicin treatment on ABR readouts at each of three different hearing frequencies (4, 10, and 20 kHz) in guinea pigs treated with 80 mg/kg gentamicin. FIG. 21A shows the results of treatment with vehicle; FIG. 21B shows the results of treating with gentamicin for 1 day; FIG. 21C shows the results of treatment with gentamicin for 3 days; FIG. 21D shows the results of treatment with gentamicin for 5 days; and FIG. 21E shows the results of treatment with gentamicin for 14 days. No significant effects on hearing were observed after 1, 3, or 5 days of treatment, whereas a significant effect on hearing was observed after 14 days of treatment.

FIG. 22 is a graphical representation of data from a mathematical model comparing the amount of gentamicin accumulated in the rat kidney cortex after administration by infusion over 1 minute, 10 minutes, or 30 minutes of a total dose of 250 μg to a 250 g rat.

### DETAILED DESCRIPTION

The present invention provides new methods for treating bacterial infections using aminoglycosides. As demonstrated in the accompanying Examples, the methods of the present invention provide new dosing regimens that are associated with decreased toxicity and enhanced efficacy as compared to previous aminogycoside dosing regimens. The dosing regimens of the present invention achieve effective aminoglycoside pharmacokinetic profiles by utilizing one or more of the following dosing parameters: (1) administration of a high dosage of an aminoglycoside; (2) administration of an aminoglycoside not more than once per day; (3) administration of an aminoglycoside for short duration of time, e.g., 5 days or less; and (4) administration of an aminoglycoside at a fast infusion rate. The present invention includes dosing regimens based on any of these parameters, alone or in any combination. As described herein, administration of a high dosage of an aminoglycoside and/or administration of an aminoglycoside at a fast infusion rate leads to a higher Cₘₐₓ of the administered aminoglycoside as compared to previous lower dosage or slower infusion rates. This resulting higher Cₘₐₓ is associated with enhanced efficacy. Furthermore, administration of an aminoglycoside less frequently or administration of an aminoglycoside over a short duration of time results in less associated toxicity.

According to the present invention, one or more of the dosing parameters described above, considered alone or in combination with each other, as well as in combination with other parameters or features (e.g., formulation), may be selected to achieve a clinically significant pharmacokinetic profile for an administered aminoglycoside—including, for example, without resulting in the toxicities typically associated with aminoglycosides, such as nephrotoxicity and ototoxicity. In particular embodiments of the dosing regimens of the present invention, two or more (i.e., two, three or four) of the dosing parameters described above can be used in combination to achieve therapeutic efficacy without associated toxicity.

In particular embodiments, the dosing regimens of the present invention provide an aminoglycoside at a higher dosage and for a shorter duration of time than previous standard dosing regimens. The higher dosage provides enhanced efficacy, while the shorter duration of treatment result in reduced nephrotoxicity or ototoxicity, as compared to previous dosing regimens that used a lower dosage for a longer period of time. In addition, dosing regimens of the present invention may provide an aminoglycoside using relatively higher intravenous infusion rates than previous dosing regimens. These higher infusion rates also provide increased efficacy and reduced toxicity as compared to current clinical dosing regimens.

Without wishing to be bound by any one theory, it is believed that the dosing regimens of the present invention, even though they use relatively high dosages of aminoglycoside or achieve relatively high Cₘₐₓ levels of aminoglycoside, result in decreased nephrotoxicity due to the shorter time-frame in which the kidneys of a subject being treated are exposed to the aminoglycoside. As discussed above, aminoglycosides are taken up by proximal tubule cells through an active pinocytosis process facilitated by the brush border binding to megalin. The rate of megalin-receptor mediated uptake is relatively fast, but this uptake is a saturable process that follows Michaelis-Menton kinetics with a measurable constant (Kₘ) associated with the saturation level. In the rat, the saturation constant Kₘ of gentamicin corresponds to approximately 15 μg/ml in the serum. The clearance half-life from the renal cells is considerably slower than uptake into the cell. Therefore, high dosages above the saturation level do not lead to increased nephrotoxicity as compared to lower dosages that meet the saturation level. However, since aminoglycoside bactericidal activity is concentration-dependent, such high dosages of aminoglycoside cause more effective (e.g., faster and/or effecting more members of the population, more extensive, “deeper”) killing of exposed bacteria, at least within the clinically relevant dosage range, including several multiples above the minimum inhibitory concentration (MIC). Enhanced time-to-kill and depth-of-kill may also translate into improved long-range effectiveness of aminoglycosides as antibiotic agents, since treated strains will have reduced opportunity to develop resistance to the aminoglycoside used for treatment.

In addition, the shorter time-frames and/or reduced frequency in which subjects are treated according to the dosing regimens of the present invention result in less cumulative exposure of the kidneys to the aminoglycoside, as compared to previous dosing regimens in which a subject was administered lower dosages more frequently and for a longer duration, in order to maintain the aminoglycoside serum concentration above the MIC of the infecting organism for as long as possible throughout the dosing interval. This known approach included various dosing frequencies, such as once per day (QD), twice per day (BID), or three times per day (TID, e.g., every eight hours), but in each case using relatively low doses, long infusion times (e.g., typically one hour), and a long course of treatment (e.g., typically seven to twenty-one days), leading to prolonged exposure to the aminoglycoside and risk of nephrotoxicity. Without wishing to be bound by theory not expressly recited in the claims, such known dosage regimens substantially saturate renal proximal tubule cells over the entire course of treatment, and significantly, only a fraction of the administered aminoglycoside contributes to a concentration in excess of the kidney saturation concentration.

In the following description, certain specific details are set forth in order to provide a thorough understanding of various embodiments of the invention. However, one skilled in the art will understand that the invention may be practiced without these details.

Unless the context requires otherwise, throughout the present specification and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Reference to a range of values, e.g., “ranges from x value to y value” or “in the range of x value to y value” are understood to be inclusive of x value and y value.

### A. Aminoglycoside Dosing Regimens for Treating Bacterial Infection

In certain embodiments, the present invention provides methods for treating a bacterial infection in a subject comprising administering an effective amount of an aminoglycoside to the subject according to a dosing regimen based upon one or more of the dosing parameters described above.

As described herein and exemplified in the accompanying Examples, the administered dosage may be determined based on one or more of several criteria and approaches.

For example, the administered dosage may be determined based upon achieving a desired pharmacokinetic profile (e.g., a serum pharmacokinetic profile) or toxicity profile. In particular embodiments, for example, dosage may be based upon achieving a maximum serum concentration (Cₘₐₓ) well above the minimum inhibitory concentration (e.g., a minumum inhibitory concentration (90%)) of an aminoglycoside, achieving a Cₘₐₓ and a pharmacokinetic profile defined by a time-concentration curve such that the ratio of Cₘₐₓ to the total area under the time-concentration curve (AUC) achieves a particular level, or achieving a time-concentration curve, such that at least 30% AUC is an area above a kidney saturation concentration, C_{KS}, for the aminoglycoside. In particular embodiments for example, the administered dosage may be determined based upon maintaining substantially baseline renal function as indicated by one or more nephrotoxicty markers, and/or upon maintaining substantially baseline auditory function as indicated by one or more auditory markers.

As another example, in particular embodiments, the dosage is a high dose regimen. In various embodiments, the dosage used may be a potency-normalized amount of aminoglycoside and/or a toxicity-normalized amount of aminoglycoside.

In various embodiments, the subject is administered an aminoglycoside for not more than 7 days, not more than 6 days, not more than 5 days, not more than 4 days, not more than 3 days, not more than 2 days, or not more than 1 day. As used herein, the phrase “not more than [any number of] days (or hours)” indicates consecutive days or hours. For example, the present invention includes dosing regimens that provide for administration of an aminoglycoside not more than once per day for a short duration of time, e.g., not more than three days, four days, or five days. In particular embodiments, methods of the present invention comprise administering to a subject a high dosage of one or more aminoglycosides for a short duration of time (high dose, short course). In particular embodiments, the subject is administered a high dosage of aminoglycoside not more than once per 24 hours, not more than once per 36 hours, not more than once per 48 hours, or not more than once per 72 hours. Various embodiments of these and other methods of the present invention are described in further detail below.

As used herein, “treating” or “treatment” covers the treatment of the disease or condition of interest in a mammal, preferably a human, having the disease or condition of interest, and includes:

(i) preventing the disease or condition from occurring in a mammal, in particular, when such mammal is predisposed to the condition but has not yet been diagnosed as having it;

(ii) inhibiting the disease or condition, i.e., arresting its development;

(iii) relieving the disease or condition, i.e., causing regression of the disease or condition; or

(iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

“Effective amount” or “therapeutically effective amount” refers to that amount of a compound of the invention which, when administered to a mammal, preferably a human, is sufficient to effect treatment, as defined above, of a bacterial infection in the mammal, preferably a human. For example, an effective amount of an aminoglycoside may be an amount effective for treating a bacterial infection as determined by clinical response or eradication of the bacteria from the site of infection in the subject. The amount of a compound of the invention which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Mammal” includes humans and both domestic animals such as laboratory animals and household pets (e.g., cats, dogs, swine, cattle, sheep, goats, horses, rabbits), and non-domestic animals such as wildlife and the like.

1. Potency and Toxicity Normalized Dosing Regimens

Certain dosing regimens of the present invention are demonstrated in the accompanying Biological Examples using gentamicin, a known aminoglycoside. According to the present invention, gentamicin dosing regimens may be adapted for the use of any aminoglycoside, e.g., based upon the relative potency and/or toxicity of the aminoglycoside as compared to gentamicin. The present invention includes dosing regimens, e.g., high dose, short course dosing regimens, based upon an administered aminoglycoside's relative potency and/or toxicity as compared to gentamicin.

The administered dose can be at least an amount sufficent to have bactericidal activity against the bacteria type (e.g., strain or species) infecting the subject. In one embodiment, the present invention provides a method for treating a bacterial infection in a subject, the method comprising administering an effective amount of an aminoglycoside to the subject not more than once per day for not more than five days, the effective amount being a potency-normalized amount of at least N_{GEN}×9 mg/kg/day, where N_{GEN}=MIC_{AG}/MIC_{GEN} is a normalizing factor defined by the ratio of a minimum inhibitory concentration (e.g., a minumum inhibitory concentration (90%)) of the administered aminoglycoside, MIC_{AG}, to a minimum inhibitory concentration (e.g., a minumum inhibitory concentration (90%)) of gentamicin, MIC_{GEN}. In other related embodiments, the subject is administered a potency-normalized amount of at least N_{GEN}×7 mg/kg/day, at least N_{GEN}×8 mg/kg/day, at least N_{GEN}×10 mg/kg/day, at least N_{GEN}×11 mg/kg/day, at least N_{GEN}×12 mg/kg/day, at least N_{GEN}×13 mg/kg/day, at least N_{GEN}×15 mg/kg/day, at least N_{GEN}×20 mg/kg/day, at least N_{GEN}×25 mg/kg/day, at least N_{GEN}×30 mg/kg/day, at least N_{GEN}×40 mg/kg/day, or at least N_{GEN}×50 mg/kg/day. In certain embodiments, the effective amount is a potency normalized amount ranging between N_{GEN}×9 mg/kg/day and N_{GEN}×30 mg/kg/day (inclusive) or a potency normalized amount ranging between N_{GEN}×9 mg/kg/day and N_{GEN}×20 mg/kg/day (inclusive). In one particular embodiment, the effective amount is a potency-normalized amount of at least N_{GEN}×15 mg/kg/day. In particular embodiments, the effective amount is administered intraveneously during an infusion period less than or equal to 30 minutes, less than or equal to 15 minutes, or less than or equal to 10 minutes.

As used herein, the term “MIC,” which stands for minimum inhibitory concentration, or “MIC (90%),” refers to that concentration, in μg/mL, of a compound, e.g., an aminoglycoside, that inhibits the growth or proliferation of a strain of bacteria by at least 90% compared to an untreated control. For example, the MIC (90%) may be that concentration of a compound that inhibits 90% of tested clinical isolates of a single strain of bacteria. In a further approach, the MIC (90%) of a compound may also refer to that concentration that inhibits the growth of 90% of tested bacteria, e.g., inhibits 90% of tested bacterial strains (i.e., two or more strains, e.g., three strains, including multiple isolates of each strain). Similarly, the “MIC (50%)” refers to that concentration, in μg/mL, of a compound, e.g., an aminoglycoside, that inhibits the growth of a strain of bacteria by at least 50% compared to an untreated control. For example, the MIC (50%) may be that concentration of a compound that inhibits 50% of tested clinical isolates of a single strain of bacteria. In a further approach, the MIC (50%) of a compound may also refer to that concentration that inhibits the growth of 50% of tested bacteria, e.g., 50% of tested bacterial strains (i.e., two or more strains, e.g., three strains, including multiple isolates of each strain). With respect to any of the above-mentioned approaches, one can determine the extent to which a compound inhibits growth of a bacterial strain or isolate by observation using the unaided eye as compared to an untreated control. Alternatively, one can use quantitative measurement to determine the extent to which a compound inhibits growth of a bacterial strain or isolate.

In particular embodiments, N_{GEN} may be determined based on MIC_{AG} and MIC_{GEN} for a particular bacterial species (e.g., a single strain or multiple strains thereof), a particular bacterial strain (e.g., a single isolate or multiple isolates thereof), or a set of bacterial strains (e.g., two or more strains, which may include multiple isolates of each). Preferably, MIC values are determined using one or more bacterial strains that are not resistant to the administered aminoglycoside or gentamicin. For example, MIC values may be determined using one or more standard susceptible (non-resistant) bacterial strains. Exemplary strains that may be used to determine MIC values include the E. coli ATCC strain 25922, the P. aeruginosa ATCC strain 27853, and the S. aureus ATCC strain 29213, each of which is available from American Type Culture Collection (ATCC; Manassas, Va., USA). N_{G} EN may alternatively be determined based on MIC_{AG} and MIC_{GEN} for the bacteria type or bacterium infecting the subject, e.g., the bacterial species, bacterial strain, or bacterial clinical isolate infecting a subject and for which the subject is being treated.

MIC values may be determined by routine methods available in the art, including the Clinical and Laboratory Standards Institute (CLSI) broth microdilution per M7-A7 as described in Clinical and Laboratory Standards Institute (2006) Methods for Dilution Antimicrobial Susceptibility Tests for Bacteria That Grow Aerobically; approved standard—seventh edition, Wayne, Pa.: CLSI. For example, inhibition of bacterial growth may be determined using the unaided eye or by quantitative measurement, e.g., by measuring optical density of a bacterial sample and comparing it to a standard reference curve. In particular embodiments, the MIC is determined as the average of three or more independent tests.

The administered dose can be an amount equal to or less than an amount which would have clinically relevant toxic (e.g., nephrotoxic) effect on the subject. In an embodiment, the present invention provides a method for treating a bacterial infection in a subject, the method comprising administering an effective amount of an aminoglycoside to the subject not more than once per day for not more than five days, the effective amount being equal to or less than a toxicity-normalized amount of T_{GEN}×50 mg/kg/day, where T_{GEN}=MTC_{AG}/MTC_{GEN} is a normalizing factor defined by the ratio of a minimum toxic concentration of the administered aminoglycoside, MTC_{AG}, to a minimum toxic concentration or dose of gentamicin, MTC_{GEN}. When determining MTC values using in vivo animal models of toxicity or data from human clinical trials, it may be preferable to measure MTCs as a minimum toxic dose. When determining MTC values using in vitro assays, MTCs may be measured as either minimum toxic concentrations or minimum toxic doses, so long as the same method is used to determine the MTC_{AG} and MTC_{GEN} when determining a T_{GEN} value.

In particular embodiments, the effective amount is a toxicity-normalized amount equal to or less than T_{GEN}×40 mg/kg/day, equal to or less than T_{GEN}×30 mg/kg/day, equal to or less T_{GEN}×25 mg/kg/day, equal to or less than a toxicity-normalized amount of T_{GEN}×20 mg/kg/day, or equal to or less than a toxicity-normalized amount of T_{GEN}×15 mg/kg/day. In certain embodiments, the effective amount is a toxicity normalized amount ranging between T_{GEN}×15 mg/kg/day and T_{GEN}×30 mg/kg/day, ranging between T_{GEN}×15 mg/kg/day and T_{GEN}×50 mg/kg/day, ranging between T_{GEN}×30 mg/kg/day and T_{GEN}×50 mg/kg/day, ranging between T_{GEN}×15 mg/kg/day and T_{GEN}×100 mg/kg/day, ranging between T_{GEN}×7 mg/kg/day and T_{GEN}×50 mg/kg/day, ranging between T_{GEN}×9 mg/kg/day and T_{GEN}×50 mg/kg/day, ranging between T_{GEN}×12 mg/kg/day and T_{GEN}×50 mg/kg/day, ranging between T_{GEN}×7 mg/kg/day and T_{GEN}×100 mg/kg/day, or ranging between T_{GEN}×9 mg/kg/day and T_{GEN}×100 mg/kg/day. In particular embodiments, the effective amount is adminstered intraveneously during an infusion period less than or equal to 30 minutes, less than or equal to 15 minutes, or less than or equal to 10 minutes.

MTC may be determined using in vivo and in vitro assays. For example, MTC may be determined using an animal model of nephrotoxicity, such as the rat nephrotoxicity assay described in Biological Example 2. According to this assay, MTC refers to the minimum dose that results in a 25% increase in a serum marker of glomerular filtration rate (GFR) or a stastically significant increase (P<0.05) as compared to a baseline level of the serum marker of GFR, as measured using the rat nephrotoxicity assay. Preferably, the rat nephrotoxicity assay utilized to determine MTC comprises once-daily subcutaneous dosing, at a 1 mL/kg dosing volume of the aminoglycoside formulated in water to an adult Sprague-Dawley rat. MTC may also be readily determined using a dog nephrotoxicity model system of analogous design. In particular embodiments, the serum marker of glomerular filtration is blood urea nitrogen (BUN) or serum creatine. The level of the GFR marker may be determined at the conclusion of 5 days of dosing with the aminoglycoside or, alternatively, at the end of 7, 11, or 14 days of dosing with the aminoglycoside. In particular embodiments, the MTC is determined as the average of values obtained from three or more animals.

MTC and T_{GEN} can alternatively be defined using a cell-based system, where the minimum toxic concentration is defined by the lowest dose or concentration that produces a relevant, detectable signal of toxicity in that system, such as the cell-based system is described in U.S. Patent Application Publication No. 20090220982. In one embodiment, this cell-based method of determining nephrotoxicity comprises: (i) contacting discrete populations of HK-2 cells with an administered aminoglycoside or gentamicin; wherein a plurality of different concentrations of the aminoglycoside or gentamicin contacts a separate discrete population of HK-2 cells; (ii) determining the level of an indicator of nephrotoxicity for each of said populations of HK-2 cells contacted in step (i) to produce a dose response curve for the aminoglycoside and dose response curve for gentamicin; and (iii) determining the determining the dose or concentration that produces a 10% or 20% increase in the indicator of nephrotoxicity for each of the administered aminoglycoside and gentamicin, thus determining MTC_{AG} and MTC_{GEN}, respectively. In particular embodiments, the indicator of nephrotoxicity is apoptosis or activity of a caspase.

T_{GEN} can also be defined using the comparative clinically relevant toxicity of the administered aminoglycoside compared to a gentamicin control in human clinical trials where T_{GEN}=MTC_{AG}/MTC_{GEN}. In such a comparison, MTC refers to the minimum dose that results in a 25% increase in a serum marker of glomerular filtration rate (GFR) or a statistically significant increase (P<0.05) as compared to a baseline level of the serum marker of GFR for the administered aminoglycoside (MTC_{AG}) and gentamicin (MTC_{GEN}). When this approach is used to determine MTC based upon data from a population of patients, MTC refers to the minimum dose that results in a 25% increase in a serum marker of GFR or a statistically significant increase (P<0.05) as compared to a baseline level of the serum marker of GFR) in a statistically significant (P<0.05) number of patients as compared to control.

Preferably, the administered dose can be at least an amount sufficient to have bactericidal activity against the bacteria type (e.g., strain or species) infecting the subject, and an amount equal to or less than an amount which would have clinically relevant toxic (e.g., nephrotoxic) effect on the subject. Hence, in related embodiments, the present invention provides a method for treating a bacterial infection in a subject, the method comprising administering an effective amount of an aminoglycoside to the subject not more than once per day for not more than five days, wherein the effective amount is equal to or greater than a potency-normalized amount of N_{GEN}×9 mg/kg/day and wherein the effective amount is equal to or less than a toxicity-normalized amount of T_{GEN}×50 mg/kg/day. In particular related embodiments, the subject is administered a potency-normalized amount of at least N_{GEN}×7 mg/kg/day, at least N_{GEN}×8 mg/kg/day, at least N_{GEN}×10 mg/kg/day, at least N_{GEN}×11 mg/kg/day, at least N_{GEN}×12 mg/kg/day, at least N_{GEN}×13 mg/kg/day, at least N_{GEN}×15 mg/kg/day, at least N_{GEN}×20 mg/kg/day, at least N_{GEN}×25 mg/kg/day, or at least N_{GEN}×30 mg/kg/day and a toxicity-normalized amount equal to or less than T_{GEN}×100 mg/kg/day, equal to or less than T_{GEN}×50 mg/kg/day, equal to or less than T_{GEN}×40 mg/kg/day, equal to or less than T_{GEN}×30 mg/kg/day, equal to or less T_{GEN}×25 mg/kg/day, equal to or less than a toxicity-normalized amount of T_{GEN}×20 mg/kg/day, or equal to or less than a toxicity-normalized amount of T_{GEN}×15 mg/kg/day. In particular embodiments, the effective amount is administered during an infusion period less than or equal to 30 minutes, less than or equal to 15 minutes, or less than or equal to 10 minutes.

Accordingly, in particular embodiments, the effective amount adminstered to a subject is: equal to or greater than a potency-normalized amount of N_{GEN}×7 mg/kg/day and equal to or less than a toxicity-normalized amount of T_{GEN}×50 mg/kg/day; equal to or greater than a potency-normalized amount of N_{GEN}×8 mg/kg/day and equal to or less than a toxicity-normalized amount of T_{GEN}×50 mg/kg/day; equal to or greater than a potency-normalized amount of N_{GEN}×9 mg/kg/day and equal to or less than a toxicity-normalized amount of T_{GEN}×50 mg/kg/day; equal to or greater than a potency-normalized amount of N_{GEN}×10 mg/kg/day and equal to or less than a toxicity-normalized amount of T_{GEN}×50 mg/kg/day; equal to or greater than a potency-normalized amount of N_{GEN}×11 mg/kg/day and equal to or less than a toxicity-normalized amount of T_{GEN}×50 mg/kg/day; equal to or greater than a potency-normalized amount of N_{GEN}×12 mg/kg/day and equal to or less than a toxicity-normalized amount of T_{GEN}×50 mg/kg/day; equal to or greater than a potency-normalized amount of N_{GEN}×13 mg/kg/day and equal to or less than a toxicity-normalized amount of T_{GEN}×50 mg/kg/day; equal to or greater than a potency-normalized amount of N_{GEN}×15 mg/kg/day and equal to or less than a toxicity-normalized amount of T_{GEN}×50 mg/kg/day; equal to or greater than a potency-normalized amount of N_{GEN}×20 mg/kg/day and equal to or less than a toxicity-normalized amount of T_{GEN}×50 mg/kg/day; equal to or greater than a potency-normalized amount of N_{GEN}×25 mg/kg/day and equal to or less than a toxicity-normalized amount of T_{GEN}×50 mg/kg/day; equal to or greater than a potency-normalized amount of N_{GEN}×30 mg/kg/day and equal to or less than a toxicity-normalized amount of T_{GEN}×50 mg/kg/day; equal to or greater than a potency-normalized amount of N_{GEN}×9 mg/kg/day and equal to or less than a toxicity-normalized amount of T_{GEN}×40 mg/kg/day; equal to or greater than a potency-normalized amount of N_{GEN}×9 mg/kg/day and equal to or less than a toxicity-normalized amount of T_{GEN}×30 mg/kg/day; equal to or greater than a potency-normalized amount of N_{GEN}×9 mg/kg/day and equal to or less than a toxicity-normalized amount of T_{GEN}×20 mg/kg/day; or equal to or greater than a potency-normalized amount of N_{GEN}×9 mg/kg/day and equal to or less than a toxicity-normalized amount of T_{GEN}×15 mg/kg/day.

2. Maximum Serum Concentration (Cₘₐₓ) Dosing Regimens

The present invention also includes high dose, short course and/or fast-infusion dosing regimens based upon achieving a serum concentration of the administered aminoglycoside well above its minimum inhibitory concentration (e.g., a minumum inhibitory concentration (90%)). These dosing regimens may be based solely on Cₘₐₓ or they may be based upon Cₘₐₓ in combination with one or more additional pharmacokinetic parameters, e.g., parameters of a serum pharmacokinetic profile. The term “Cₘₐₓ” indicates the maximum plasma concentration of a compound, e.g., an aminoglycoside, reached following administration.

The administered dose can be at least an amount sufficent to realize a high Cₘₐₓ relative to the minimum inhibitory concentration for the administered aminoglycoside. Thus, in one embodiment, the present invention provides a method for treating a bacterial infection in a subject, the method comprising administering an effective amount of an aminoglycoside to the subject not more than once per day for not more than five days to achieve a maximum serum concentration of the administered aminoglycoside, Cₘₐₓ, equal to at least 8 times the minimum inhibitory concentration (e.g., a minumum inhibitory concentration (90%)) of the administered aminoglycoside, MIC_{AG}, for the bacterial isolate infecting the subject. In related embodiments, the method is practiced using a maximum serum concentration of the administered aminoglycoside, Cₘₐₓ, equal to at least 5, at least 6, at least 7, at least 9, at least 10, at least 12, at least 15, at least 20, at least 30, at last 40, at least 50, at least 60, at least 70, at least 80, at least 90, or at least 100 times the minimum inhibitory concentration (e.g., a minumum inhibitory concentration (90%)) of the administered aminoglycoside, MIC_{AG}. In other embodiments, the ratio of Cₘₐₓ:MIC_{AG} is in the range of about 5 to about 96, about 8 to about 96, about 12 to about 96, about 16 to about 96, about 5 to about 64, about 8 to about 64, about 10 to about 64, about 12 to about 64, about 16 to about 64, about 5 to about 32, about 8 to about 32, about 10 to about 32, about 12 to about 32, about 16 to about 32, about 5 to about 16, about 8 to about 16, about 10 to about 16, about 12 to about 16, about 5 to about 12, about 8 to about 12, at least 10, at least 12, or at least 16. In another embodiment, the range is about 5 to about 150, about 8 to 150, about 12 to 150, or about 16 to 150. In particular embodiments, the effective amount is administered intraveneously during an infusion period less than or equal to 30 minutes, less than or equal to 15 minutes, or less than or equal to 10 minutes.

The administered dose can be an amount characterized by the relation of a realized Cₘₐₓ relative to the pharmacokinetic parameter of total area under the time-concentration curve for the administered aminoglycoside. In such embodiment, the present invention provides a method for treating a bacterial infection in a subject, the method comprising administering an effective amount of an aminoglycoside to the subject not more than once per day for not more than five days to achieve a maximum serum concentration of the administered aminoglycoside, Cₘₐₓ, and a pharmacokinetic profile defined by a time-concentration curve, the ratio of Cₘₐₓ to total area under the time-concentration curve, AUC, being at least 0.4 hr⁻¹. In related embodiments, the Cₘₐₓ to AUC ratio is at least 0.3 hr⁻¹, at least 0.5 hr⁻¹, at least 0.6 hr⁻¹, at least 0.7 hr⁻¹, at least 0.8 hr⁻¹, or at least 0.9 hr⁻¹. In further embodiments, the Cₘₐₓ to AUC ratio ranges from about 0.4 hr⁻¹ to about 1.0 hr⁻¹, from about 0.5 hr⁻¹ to about 1.0 hr⁻¹, from about 0.6 hr⁻¹ to about 1.0 hr⁻¹, from about 0.4 hr⁻¹ to about 0.9 hr⁻¹, from about 0.5 hr⁻¹ to about 0.9 hr⁻¹, or from about 0.6 hr⁻¹ to about 0.9 hr⁻¹. In one embodiment, the Cₘₐₓ to AUC ratio ranges from about 0.4 hr⁻¹ to about 0.7 hr⁻¹. In particular embodimets, the effective amount is adminstered intraveneously during an infusion period less than or equal to 30 minutes, less than or equal to 15 minutes, or less than or equal to 10 minutes.

In particular embodiments, the Cₘₐₓ to AUC ratio is potency-normalized and/or toxicity-normalized to gentamicin. Accordingly, in particlar embodiments, the effective amount achieves a Cₘₐₓ to AUC ratio which is a potency-normalized ratio of at least N_{GEN}×0.4 hr⁻¹. In related embodiments, the effective amount achieves a potency-normalized Cₘₐₓ to AUC ratio of at least N_{GEN}×0.3 hr⁻¹, at least N_{GEN}×0.5 hr⁻¹, at least N_{GEN}×0.6 hr⁻¹, at least N_{GEN}×0.7 hr⁻¹, at least N_{GEN}×0.8 hr⁻¹, or at least N_{GEN}×0.9 hr⁻¹. In further embodiments, the effective amount achieves a potency-normalized Cₘₐₓ to AUC ratio ranging from about N_{GEN}×0.4 hr⁻¹ to about N_{GEN}×1.0 hr⁻¹, from about N_{GEN}×0.5 hr⁻¹ to about N_{GEN}×1.0 hr⁻¹, from about N_{GEN}×0.6 hr⁻¹ to about N_{GEN}×1.0 hr⁻¹, from about N_{GEN}×0.4 hr⁻¹ to about N_{GEN}×0.9 hr⁻¹, from about N_{GEN}×0.5 hr⁻¹ to about N_{GEN}×0.9 hr⁻, or from about N_{GEN}×0.6 hr⁻¹ to about N_{GEN}×0.9 hr⁻¹. In one embodiment, the Cₘₐₓ to AUC ratio ranges from about N_{GEN}×0.4 hr⁻¹ to about N_{GEN}×0.7 hr⁻¹. In particular embodiments, the effective amount is administered intraveneously during an infusion period less than or equal to 30 minutes, less than or equal to 15 minutes, or less than or equal to 10 minutes

In other embodiments, the effective amount achieves a Cₘₐₓ to AUC ratio which is a toxicity-normalized ratio equal to or less than N_{GEN}×5.0 hr⁻¹, equal to or less than N_{GEN}×4.0 hr⁻¹, equal to or less than N_{GEN}×3.0 hr⁻, or equal to or less than N_{GEN}×2.0 hr⁻¹. In particular embodiments, the effective amount is administered intraveneously during an infusion period less than or equal to 30 minutes, less than or equal to 15 minutes, or less than or equal to 10 minutes

In related embodiments, the effective amount is an amount that achieves a potency-normalized Cₘₐₓ to AUC ratio of at least N_{GEN}×0.4 hr⁻¹, at least N_{GEN}×0.5 hr⁻¹, at least N_{GEN}×0.6 hr⁻¹, at least N_{GEN}×0.7 hr⁻¹, at least N_{GEN}×0.8 hr⁻¹, or at least N_{GEN}×0.9 hr⁻¹ and that achieves a toxicity-normalized Cₘₐₓ to AUC ratio equal to or less than N_{GEN}×5.0 hr⁻¹, equal to or less than N_{GEN}×4.0 hr⁻¹, equal to or less than N_{GEN}×3.0 hr⁻¹, or equal to or less than N_{GEN}×2.0 hr⁻¹. In particular embodiments, the effective amount is administered intraveneously during an infusion period less than or equal to 30 minutes, less than or equal to 15 minutes, or less than or equal to 10 minutes

The administered dose can be preferably determined pursuant to the guidance provided herein for a particular aminoglycoside being administered to a subject. As a non-limiting example, doses are described herein for 6′-(2-Hydroxy-ethyl)-1-(4-amino-2(S)-hydroxy-butyryl)-sisomicin.

In a particular embodiment, the present invention provides a method for treating a bacterial infection in a subject, the method comprising administering an effective amount of the compound, 6′-(2-Hydroxy-ethyl)-1-(4-amino-2(S)-hydroxy-butyryl)-sisomicin to the subject not more than once per day for not more than five days to achieve a maximum serum concentration of the compound, 6′-(2-Hydroxy-ethyl)-1-(4-amino-2(S)-hydroxy-butyryl)-sisomicin, Cₘₐₓ, equal to at least 5 or at least 8 times the minimum inhibitory concentration (e.g., a minumum inhibitory concentration (90%)) of the compound, 6′-(2-Hydroxy-ethyl)-1-(4-amino-2(S)-hydroxy-butyryl)-sisomicin, for the bacterial species, strain or isolate infecting the subject. In one embodiment, the Cₘₐₓ:MIC is in the range of about 5 to about 96. In particular embodiments, the effective amount is administered intraveneously during an infusion period less than or equal to 30 minutes, less than or equal to 15 minutes, or less than or equal to 10 minutes

In another embodiment, the present invention provides a method for treating a bacterial infection in a subject, the method comprising administering an effective amount of the compound, 6′-(2-Hydroxy-ethyl)-1-(4-amino-2(S)-hydroxy-butyryl)-sisomicin to the subject not more than once per day for not more than five days to achieve a maximum serum concentration of the compound, Cₘₐₓ, and a pharmacokinetic profile defined by a time-concentration curve, the ratio of Cₘₐₓ to total area under the time-concentration curve, AUC, being at least 0.4 hr⁻¹. In further embodiments, the Cₘₐₓ to AUC ratio is in the range from about 0.4 hr⁻¹ to about 1.0 hr⁻¹. In particular embodiments, the effective amount is administered intraveneously during an infusion period less than or equal to 30 minutes, less than or equal to 15 minutes, or less than or equal to 10 minutes.

3. Kidney Saturation Concentration Dosing Regimens

The present invention also includes dosing regimens based upon achieving a certain sustained serum concentration of bactericidally-active aminoglycoside above the kidney saturation concentration of the administered aminoglycoside. Such dosing regimen may be realized as achieving a serum pharmacokinetic profile characterized by total area under the time-concentration curve relative to the kidney saturation concentration.

Therefore, in one embodiment, the present invention provides a method for treating a bacterial infection in a subject, the method comprising administering an effective amount of an aminoglycoside to the subject not more than once per day for not more than five days to achieve a serum pharmacokinetic profile defined by a time-concentration curve, at least 30% of total area under the time-concentration curve, AUC, being an area above a kidney saturation concentration, C_{KS}, for the aminoglycoside. In other related embodiments, at least 40%, at least 50%, at least 60%, at least 70%, at least 80%, or at least 90% of total AUC is above C_{KS}. In particular embodimets, the effective amount is administered intraveneously during an infusion period less than or equal to 30 minutes, less than or equal to 15 minutes, or less than or equal to 10 minutes.

As used herein, kidney saturation concentration, C_{KS}, is the concentration of aminoglycoside at which uptake of the aminoglycoside into renal proximal tubule cells becomes saturated (i.e., the concentration above which, no further uptake of the aminoglycoside into renal proximal tubule cells is observed). Kidney saturation concentration, C_{KS}, can be determined in vitro using renal proximal tubule cells or a surrogate thereof (e.g., the HK-2 cell assay described in U.S. Patent Application Publication No. 20090220982 and above) or in vivo, e.g., using a rat model or in human clinical studies.

4. Infusion Rate Dosing Regimens

According to the present invention, faster infusion rates are associated with higher Cₘₐₓ (e.g., relative to MIC), which is associated with increased efficacy, and are relatedly associated with desirable pharmacokinetic profiles—for example, the ratio of Cₘₐₓ to AUC, or for example, the extent the total area under the time-concentration curve, AUC, being above a kidney saturation concentration, C_{KS} (in each case, as described herein). Accordingly, the present invention includes dosing regimens that utilize relatively fast infusion rates (e.g., compared to infusion rates previously used in connection with typical clinical administration of aminoglycosides). Dosing regimens of the present invention that utilize a fast infusion rate may also include the administration of high dosages and/or a short course of treatment (e.g., 5 days or less), although this is not required. In particular embodiments, a fast infusion time is associated with a high Cₘₐₓ but reduced chronic exposure, and the aminoglycoside may be administered over a longer time period without toxicity.

In one embodiment, the present invention provides a method for treating a bacterial infection in a subject, the method comprising administering an aminoglycoside to the subject at a high infusion rate. In particular embodiments, the infusion period is not more than one hour, not more than 30 minutes, not more than 20 minutes, not more than 15 minutes, not more than 10 minutes, not more than 5 minutes, not more than 2 minutes, or no more than 1 minute. In related embodiments, the infusion period is between 1 and 5 minutes, between 1 and 10 minutes, between 1 and minutes, between 1 and 20 minutes, between 5 and 20 minutes, between 5 and 15 minutes, between 5 and 10 minutes, between 10 and 20 minutes, between 10 and 15 minutes, about 10 minutes, or about 15 minutes. In addition, the aminoglycoside may be administered not more than once per day for at least one day, at least two days, at least three days, at least four days, or at least five days.

Thus, in one embodiment, the present invention provides a method for treating a bacterial infection in a subject, the method comprising administering an effective amount of an aminoglycoside to the subject not more than once per day for at least two days by intravenous infusion over an infusion period less than or equal to 15 minutes. In related embodiments, the infusion period is not more than 30 minutes, not more than 20 minutes, not more than 15 minutes, not more than 10 minutes, not more than 5 minutes, or not more than 1 minute. In particular embodiments, the aminoglycoside is administered over an infusion period of about 10 minutes, between 1 and 10 minutes, between 1 and 15 minutes, or between 1 and 30 minutes. In particular embodiments of methods of the present invention, an aminoglycoside is administered at a constant rate over the infusion period or a majority of the infusion period.

In a further embodiment of the high infusion rate method described above, the present invention includes a method for treating a bacterial infection in a subject, the method comprising administering an effective amount of an aminoglycoside to the subject not more than once per day for at least two days by intravenous infusion with an infusion rate of at least N_{GEN}×0.5 mg/kg/min, where N_{GEN}=MIC_{AG}/MIC_{GEN} is a normalizing factor defined by the ratio of a minimum inhibitory concentration (e.g., a minumum inhibitory concentration (90%)) of the administered aminoglycoside, MIC_{AG}, to a minimum inhibitory concentration (e.g., a minumum inhibitory concentration (90%)) of gentamicin, MIC_{GEN}, for a bacterial strain selected from the E. coli ATCC strain 25922, the P. aeruginosa ATCC strain 27853, or the S. aureus ATCC strain 29213. In related embodiments, the infusion rate is at least N_{GEN}×0.3 mg/kg/min, N_{GEN}×0.4 mg/kg/min, N_{GEN}×0.6 mg/kg/min, N_{GEN}×0.7 mg/kg/min, or N_{GEN}×0.8 mg/kg/min.

In a related embodiment, the present invention includes a method for treating a bacterial infection in a subject, the method comprising administering an effective amount of an aminoglycoside to the subject by intravenous infusion with an infusion rate of at least N_{GEN}×0.5 mg/kg/min. In related embodiments, the infusion rate is at least N_{GEN}×0.3 mg/kg/min, N_{GEN}×0.4 mg/kg/min, N_{GEN}×0.6 mg/kg/min, N_{GEN}×0.7 mg/kg/min, or N_{GEN}×0.8 mg/kg/min.

In another embodiment, the present invention provides a high dose infusion method for treating a bacterial infection in a subject, the method comprising administering an effective amount of an aminoglycoside to the subject by intraveneous infusion, the infused amount being a potency-normalized amount of at least N_{GEN}×15 mg/kg/infusion, where N_{GEN}=MIC_{AG}/MIC_{GEN} is a normalizing factor defined by the ratio of a minimum inhibitory concentration (e.g., a minumum inhibitory concentration (90%)) of the administered aminoglycoside, MIC_{AG}, to a minimum inhibitory concentration (e.g., a minumum inhibitory concentration (90%)) of gentamicin, MIC_{GEN}, for a bacterial strain selected from the E. coli ATCC strain 25922, the P. aeruginosa ATCC strain 27853, or the S. aureus ATCC strain 29213. In related embodiments, the infused amount is at least N_{GEN}×8 mg/kg/infusion, at least N_{GEN}×9 mg/kg/infusion, at least N_{GEN}×12 mg/kg/infusion, at least N_{GEN}×15 mg/kg/infusion, at least N_{GEN}×20 mg/kg/infusion, at least N_{GEN}×25 mg/kg/infusion, at least N_{GEN}×30 mg/kg/infusion, at least N_{GEN}×40 mg/kg/infusion, or at least N_{GEN}×50 mg/kg/infusion.

In a related high dose infusion method, the present invention provides a method for treating a bacterial infection in a subject, the method comprising administering an effective amount of an aminoglycoside to the subject by intravenous infusion to achieve a maximum serum concentration of the administered aminoglycoside, Cₘₐₓ, equal to at least 8 times the minimum inhibitory concentration (e.g., a minumum inhibitory concentration (90%)) of the administered aminoglycoside, MIC_{AG}, for the bacterial isolate infecting the subject. In related embodiments, the method achieves a Cₘₐₓ, equal to at least 5, at least 6, at least 7, at least 9, at least 10, at least 12, at least 15, at least 20, at least 30, at last 40, at least 50, at least 60, at least 70, at least 80, at least 90, or at least 100 times the minimum inhibitory concentration (e.g., a minumum inhibitory concentration (90%)) of the administered aminoglycoside, MIC_{AG}, for the bacterial species, strain or isolate infecting the subject. In other embodiments, the ratio of Cₘₐₓ:MIC_{AG} is in the range of about 5 to about 96, about 8 to about 96, about 12 to about 96, about 16 to about 96, about 5 to about 64, about 8 to about 64, about 10 to about 64, about 12 to about 64, about 16 to about 64, about 5 to about 32, about 8 to about 32, about 10 to about 32, about 12 to about 32, about 16 to about 32, about 5 to about 16, about 8 to about 16, about 10 to about 16, about 12 to about 16, about 5 to about 12, about 8 to about 12, at least 10, at least 12, or at least 16. In another embodiment, the range is about 5 to about 150, about 8 to 150, about 12 to 150, or about 16 to 150.

In a further embodiment, the present invention provides a method for treating a bacterial infection in a subject, the method comprising administering an effective amount of an aminoglycoside to the subject by intravenous infusion to achieve a maximum serum concentration of the administered aminoglycoside, Cₘₐₓ, and a pharmacokinetic profile defined by a time-concentration curve, the ratio of Cₘₐₓ to total area under the time-concentration curve, AUC, being at least 0.6 hr⁻¹. In related embodiments, the Cₘₐₓ to AUC ratio is at least 0.4 hr⁻¹, at least 0.5 hr⁻¹, at least 0.7 hr⁻¹, at least 0.8 hr⁻¹, or at least 0.9 hr⁻¹. In further embodiments, the Cₘₐₓ to AUC ratio is in the range from about 0.4 hr⁻¹ to about 1.0 hr⁻¹, about 0.5 hr⁻¹ to about 1.0 hr⁻¹, about 0.6 hr⁻¹ to about 1.0 hr⁻¹, about 0.4 hr⁻¹ to about 0.9 hr⁻¹, about 0.5 hr⁻¹ to about 0.9 hr⁻¹, or about 0.6 hr⁻¹ to about 0.9 hr⁻¹. In one embodiment, the Cₘₐₓ to AUC ratio is in the range from about 0.4 hr⁻¹ to about 0.7 hr⁻¹. In particular embodiments, the amount administered is normalized as described above to achieve a potency-normalize and/or toxicity-normalized Cₘₐₓ to AUC ratio.

The present invention further provides a method for treating a bacterial infection in a human subject, the method comprising administering an effective amount of an aminoglycoside to the subject by intravenous infusion to achieve a serum pharmacokinetic profile defined by a time-concentration curve, at least 30% of total area under the time-concentration curve, AUC, being an area above a kidney saturation concentration, C_{KS}, for the aminoglycoside. In other related embodiments, at least 40%, at least 50%, at least 60%, at least 70%, at least 80%, or at least 90% of total AUC is above C_{KS}.

5. Toxicity Based Dosing Regimens

Methods of the present invention are associated with reduced nephrotoxicity and/or ototoxicity, and high dose, short course methods of the present invention, as well as other dosing regimens of the present invention, allow the administration of a higher amount of an aminoglycoside than prior dosing regimens without associated nephrotoxicity and/or ototoxicity. Thus, dosing regimens of the present invention may be based upon administering an effective dosage of an aminoglycoside while avoiding associated nephrotoxicity and/or ototoxicity.

In one embodiment, the present invention provides short course methods for treating a bacterial infection in a subject by adminstering a dosage of an aminoglycoside while maintaining substantially baseline renal function, as measured by a clinically relevant biomarker or a surrogate thereof.

In one embodiment of such a method, the present invention includes a method for treating a bacterial infection in a subject, the method comprising administering an effective amount of an aminoglycoside to the subject not more than once per day for not more than five days, and maintaining substantially baseline renal function in the subject as indicated by one or more nephrotoxicity biomarkers (including surrogate markers). In related embodiments, the aminoglycoside is administered not more than three days, not more than four days, not more than six days, or not more than seven days.

Nephrotoxicity markers that may be used include, but are not limited to, glomerular filtration rate (GFR), blood urea nitrogen (BUN) level, serum creatine level, and/or creatine clearance rate. Nephrotoxocity may also be determined based upon other biomarkers, including urinary markers, such as, e.g., lysosomal acid hydrolases, alanine aminopeptidase, gamma-glutamyl-transpeptidase, alkaline phosphatase, glucose, alanine aminopeptidase, gamma glutamyl transpeptidase, and lactate dehydrogenase. Nephrotoxicity may also be determined by histopathology. The present invention includes dosages determined based on any of these or other markers or nephrotoxicity.

In another embodiment of such method, and as described in Biological Example 6, certain methods of the present invention may be practiced without causing ototoxicity to a subject being treated with an aminoglycoside. Accordingly, the present invention also provides a method for treating a bacterial infection in a subject without causing otoxicity, or alternatively, while maintaining substantially baseline auditory function as indicated by one or more auditory markers (e.g., auditory brainstem response (ABR)). These methods can comprise administering an effective amount of an aminoglycoside to the subject to achieve a maximum serum concentration of the administered aminoglycoside, Cₘₐₓ, equal to at least 8 times the minimum inhibitory concentration (e.g., a minumum inhibitory concentration (90%)) of the administered aminoglycoside, MIC_{AG}, for the bacterial isolate infecting the subject. The aminoglycoside can be administered at least once per day. Alternatively, the aminoglycoside can be administered not more than once per day. In related embodiments, the method achieves a Cₘₐₓ, equal to at least 5, at least 6, at least 7, at least 9, at least 10, at least 12, at least 15, at least 20, at least 30, at last 40, at least 50, at least 60, at least 70, at least 80, at least 90, or at least 100 times the minimum inhibitory concentration (e.g., a minumum inhibitory concentration (90%)) of the administered aminoglycoside, MIC_{AG}, for the bacterial isolate infecting the subject. In other embodiments, the ratio of Cₘₐₓ:MIC_{AG} is in the range of about 5 to about 96, about 8 to about 96, about 12 to about 96, about 16 to about 96, about 5 to about 64, about 8 to about 64, about 10 to about 64, about 12 to about 64, about 16 to about 64, about 5 to about 32, about 8 to about 32, about 10 to about 32, about 12 to about 32, about 16 to about 32, about 5 to about 16, about 8 to about 16, about 10 to about 16, about 12 to about 16, about 5 to about 12, about 8 to about 12, at least 10, at least 12, or at least 16. In another embodiment, the range is about 5 to about 150, about 8 to 150, about 12 to 150, or about 16 to 150. In particular embodiments, the aminoglycoside is administered to the subject daily for at least 5 days, at least 6 days, at least 7 days, at least 8 days, at least 9 days, at least 10 days, at least 14 days, or at least 28 days.

Ototoxicity may be readily determined by methods known and available in the art, including those described in the accompanying Examples. For instance, ototoxicity may be determined in a subject by auditory brainstem evaluation, electronystagmography, pure tone audiometry, modified Romberg, and otoacoustic emissions testing.

6. Exemplary Methods of Treatment

In one particular embodiment, the present invention provides a method for treating a bacterial infection in a human subject, the method comprising administering an effective amount of the compound, 6′-(2-Hydroxy-ethyl)-1-(4-amino-2(S)-hydroxy-butyryl)-sisomicin, to the subject, wherein said compound is administered at a dosage of at least 10 mg/kg subject body weight not more than once per day for not more than five days. In related embodiments, the compound, 6′-(2-Hydroxy-ethyl)-1-(4-amino-2(S)-hydroxy-butyryl)-sisomicin, is administered at a dosage of at least 12 mg/kg subject body weight, at least 15 mg/kg subject body weight, at least 20 mg/kg subject body weight, at least 25 mg/kg body weight, or at least 30 mg/kg body weight. In related embodiments, the compound is administered at a dosage in the range of 10 to 50 mg/kg subject body weight, 10 to 40 mg/kg subject body weight, 10 to 30 mg/kg subject body weight, 10 to 20 mg/kg subject body weight, 15 to 50 mg/kg subject body weight, 15 to 40 mg/kg subject body weight, 15 to 30 m/kg subject body weight, or 15 to 20 mg/kg subject body weight. In one embodiment, the compound, 6′-(2-Hydroxy-ethyl)-1-(4-amino-2(S)-hydroxy-butyryl)-sisomicin, is administered at a dosage of at about 15 mg/kg subject body weight. In other related embodiments, the compound, 6′-(2-Hydroxy-ethyl)-1-(4-amino-2(S)-hydroxy-butyryl)-sisomicin, is administered for not more than four days, for not more than three days, for not more than two days, or for not more than one day.

In particular embodiments of the above method, the compound, 6′-(2-Hydroxy-ethyl)-1-(4-amino-2(S)-hydroxy-butyryl)-sisomicin, is administered by intravenous infusion. In certain embodiments, the infusion occurs over a time period between about 10 and about 15 minutes, over a time period less than or equal to 15 minutes, or over a time period less than or equal to 10 minutes. In related embodiments, the infusion occurs over a time period less than or equal to 5 minutes or less than or equal to 1 minute. In further embodiments, the infusion occurs over a time period of 1 to 10 minutes.

In one particular embodiment, the compound, 6′-(2-Hydroxy-ethyl)-1-(4-amino-2(S)-hydroxy-butyryl)-sisomicin, is administered at a dosage of about 15 mg/kg subject body weight not more than once per day for not more than five days by intravenous infusion, wherein each infusion occurs over a time period less than or equal to 15 minutes.

In one particular embodiment, the present invention provides a method for treating a bacterial infection in a human subject, the method comprising administering an effective amount of the compound, gentamicin, or an analog, stereoisomer, pharmaceutically acceptable salt or prodrug thereof, to the subject, wherein said compound is administered at a dosage of at least 10 mg/kg subject body weight not more than once per day for not more than five days. In related embodiments, the compound is administered at a dosage of at least 12 mg/kg subject body weight, at least 15 mg/kg subject body weight, at least 20 mg/kg subject body weight, at least 25 mg/kg body weight, or at least 30 mg/kg body weight. In related embodiments, the compound is administered at a dosage in the range of 10 to 50 mg/kg subject body weight, 10 to 40 mg/kg subject body weight, 10 to 30 mg/kg subject body weight, 10 to 20 mg/kg subject body weight, 15 to 50 mg/kg subject body weight, 15 to mg/kg subject body weight, 15 to 30 m/kg subject body weight, or 15 to 20 mg/kg subject body weight. In one embodiment, the compound, gentamicin, is administered at a dosage of at about 15 mg/kg subject body weight. In other related embodiments, the compound, gentamicin, is administered for not more than four days, for not more than three days, for not more than two days, or for not more than one day.

In particular embodiments of the above method, the compound, gentamicin, is administered by intravenous infusion. In certain embodiments, the infusion occurs over a time period between about 10 and about 15 minutes, over a time period less than or equal to 15 minutes, or over a time period less than or equal to 10 minutes. In related embodiments, the infusion occurs over a time period less than or equal to 5 minutes or less than or equal to 1 minute. In further embodiments, the infusion occurs over a time period of 1 to 10 minutes. In particular embodiments, the infusion occurs over a time period of 1 to 30 minutes, 1 to 15 minutes, or 1 to 10 minutes.

In one particular embodiment, the compound, gentamicin, is administered at a dosage of about 15 mg/kg subject body weight not more than once per day for not more than five days by intravenous infusion, wherein each infusion occurs over a time period less than or equal to 15 minutes.

In other specific examples of embodiments of the present invention, methods of the invention are used to treat: a respiratory tract infection by Streptococcus with levofloxacin; a respiratory or urinary tract infection by P. aeuroginosa with ciprofloxacin; or a urinary tract infection with E. coli with ciprofloxacin.

In additional embodiments, methods of the present invention are used to treat a subject infected with an Enterobacteriacae, including those strains that express extended spectum β-lactamases (ESBLs), metallo-β-lactamases, DNA gyrase mutations, and Klebsiella pneumoniae carbapenemases (KPCs) with the compound shown in Representative Compounds Example 1.

In further embodiments, methods of the present invention are used to treat a subject infected with Staphylococcus aureus, including strains resistant to methicillin and vancomycin (i.e., methicillin-resistance S. aureus (MRSA) and vancomycin-resistant S. aureus (VRSA) with the compound shown in Representative Compounds Example 1.

In related embodiments, methods of the present invention are used to treat a urinary tract infection (UTI) with the compound shown in Representative Compounds Example 1. For example, in certain embodiments, the compound shown in Representative Compounds Example 1 may be administered intravenously to treat a patient having a complicated UTI, or it may be administered intramuscularly to treat uncomplicated UTI. In one embodiment, the compound of Representative Compounds Example 1 is administered to treat UTI at a high dose no more than once per day for no more than 5 days, and preferably no more than 3 days. In particular embodiments, each dosage administered is at least 10 mg/kg, at least 15 mg/kg, at least 20 mg/kg, at least mg/kg, at least 30 mg/kg, at least 35 mg/kg, at least 40 mg/kg, or at least 50 mg/kg.

In some embodiments, methods of the present invention are used to treat a K. pneumoniae infection (KP) with the compound shown in Example 1. In one embodiment, the compound of Representative Compounds Example 1 is administered to treat KP at a high dose no more than once per day for no more than 5 days, and preferably no more than 3 days. In particular embodiments, each dosage administered is at least 10 mg/kg, at least 15 mg/kg, at least 20 mg/kg, at least 25 mg/kg, at least 30 mg/kg, at least 35 mg/kg, at least 40 mg/kg, or at least 50 mg/kg.

7. General Dosing Parameters

As described above, in particular embodiments, methods of the present invention contemplate administering a high dose of aminoglycoside not more than once per day for not more than five days. In particular embodiments, the dosage administered is at least 2×, at least 3×, at least 4×, at least 5×, or at least 10× the dosage previously approved by the USFDA for an approved aminoglycoside for a particular indication. For example, gentamicin and tobramycin are typically administered initially at a concentration of 2-3 mg/kg body weight, followed by a maintenance dosage of 1.5-2 mg/kg body weight every eight hours. Thus, according to certain embodiments of the present invention, gentamicin and tobramycin may be admininstered at a concentration of at least 4-6, 5-7, 6-8 mg/kg body weight not more than once per day for not more than five consecutive days. Amikacin and kanamycin are typically administered initially at a concentration of 7.5-9 mg/kg body weight, with amikacin being followed by a maintenance dosage of 5-7.5 mg/kg body weight every 12 hours. Thus, according to certain embodiments of the present invention, amikacin and kanamycin may be administered at a concentration of at least 10-15, 12-18, or 15-20 mg/kg body weight not more than once per day for not more than five consecutive days.

In particular embodiments of each of the methods described herein, the aminoglycoside is administered to the subject for not more than five days, not more than four days, or not more than three days.

According to particular embodiments of the methods of the present invention, the aminoglycoside is administered to the subject not more than once per day.

In particular embodiments of the methods described above, the aminoglycoside is administered intravenously, e.g., as a bolus injection or by intraveneous infusion.

In particular embodiments of the methods of the present invention, the aminoglycoside is 6′-(2-Hydroxy-ethyl)-1-(4-amino-2(S)-hydroxy-butyryl)-sisomicin, or a stereoisomer, pharmaceutically acceptable salt, or prodrug thereof.

In particular embodimentsm, the subject is a mammal, prefereably a human. In certain embodiments, the subject is diagnosed with or at risk of developing a bacterial infection.

### B. Indications and Infectious Agents

The methods of the present invention may be used to treat any bacterial infection. Accordingly, in particular embodiments, the aminoglycoside is administered in an amount effective for treating an infection by any such bacterium. In particular embodiments, the bacterium is a Gram negative bacteria. In other embodiments, it is a Gram positive bacterium.

In particular embodiments of the above method, the subject is infected by Enterobacteriaceae bacteria. In other particular embodiments, said subject is infected by E. coli, Pseudomonas aeruginosa, K. peumoniae, Staphylococcus saprophyticus, or Proteus mirabilis. In further embodiments, the subject is infected with a drug-resistant bacteria, which may be a multi-drug-resistant bacteria.

In particular embodiments, the aminoglycoside is administered in an amount effective for treating an infection of an Enterobacteriaceae bacteria (including wildtype (WT) strains and those with ESBL, AmpC, KPC, NMC, SME and MBL enzymes), a K. pneumonia bacteria, an E. coli bacteria, an Enterobaccter bacteria, or a Staphylococcus aureus bacteria.

In other embodiments, the aminoglycoside is administered in an amount effective for treating an infection of a bacterial strain having resistance to at least one antibacterial agent. In one embodiment, the bacterial strain comprises an aminoglycoside-resistance mechanism. In one embodiment, the bacterial strain expresses an aminoglycoside-modifying enzyme (AME) associated with aminoglycoside resistance. In certain embodiments, the bacterial strain expresses one or more of a β-lactamase, metallo-β-lactamase, mutated DNA gyrase, or a Klebsiella pneumoniae carbapenemase. In one particular embodiment, the bacterial strain is a S. aureus strain resistant to methicillin (MRSA). In another particular embodiment, the bacterial strain is a S. aureus strain resistant to vancomycin (VRSA). S. aureus and coagulase-negative staphylococci (CoNS) include oxacillin-susceptible (MSSA/MS-CONS) and —R (MRSA/MR-CoNS) strains. In particular embodiments, the bacteria is a gram-negative (e.g., Enterobacteriaceae, P. aeruginosa, Acinetobacter) or gram-positive (e.g., S. aureus) organism with or without aminoglycoside resistance mechansisms (AGRM). In one embodiment, the bacteria is a coagulase-negative staphylococci.

Examples of bacterial infections that may treated according to methods of the invention include, but are not limited to, infection by: Acinetobacter baumannii; Acinetobacter lwoffii; Baciccis Antracis; Enterobacter aerogenes; Enterobacter cloacae; Enterococcus faecalis; Corynebacterium; diphtheriae; Escherichia coli; Enterococcus faecium; Streptococcus coelicolor; Streptococcus pyogenes; Streptobacillus moniliformis; Streptococcus agalactiae; Streptococcus pneumoniae; Salmonella typhi; Salmonella paratyphi; Salmonella schottmulleri; Salmonella hirshfeldii; Staphylococcus epidermidis; Staphylococcus aureus; Klebsiella pneumoniae; Klebsiella oxytoca, Legionella pneumophila; Helicobacter pylori; Moraxella catarrhalis, Morganella morganii; Mycoplasma pneumonia; Mycobacterium tuberculosis; Mycobacterium leprae; Yersinia enterocolitica; Serratia marcenscens; Yersinia pestis; Vibrio cholerae; Vibrio parahaemolyticus; Rickettsia prowazekii; Rickettsia rickettsii; Rickettsia akari; Clostridium difficile; Clostridium tetani; Clostridium perfringens; Clostridium novyii; Clostridium septicum; Clostridium botulinum; Legionella pneumophila; Hemophilus influenzae; Hemophilus parainfluenzae; Hemophilus aegyptus; Chlamydia psittaci; Chlamydia trachomatis; Bordetella pertusis; Shigella spp.; Campylobacter jejuni; Proteus spp.; Proteus mirabilis; Citrobacter spp.; Citrobacter braakii; Citrobacter freundii; Citrobacter koseri; Enterobacter spp.; Pseudomonas aeruginosa; Propionibacterium spp.; Providencia rettgeri; Providencia stuartii; Proteus vulgaris; Bacillus anthracis; Pseudomonas syringae; Spirrilum minus; Neisseria meningitidis; Listeria monocytogenes; Neisseria gonorrheae; Treponema pallidum; Francisella tularensis; Brucella spp.; Borrelia recurrentis; Borrelia hermsii; Borrelia turicatae; Borrelia burgdorferi; Mycobacterium avium; Mycobacterium smegmatis; Staphylococcus saprophyticus; Staphyloccus aureus; Methicillin-resistant Staphyloccus aureus; heterogeous vancomycin-intermediate strains of Staphyloccus aureus; Vancomycin-resistant Staphyloccus aureus; Vancomycin-resistant enterococcus; and multi-drug resistant bacteria (e.g., bacteria that are resistant to more than 1, more than 2, more than 3, or more than 4 different drugs).

In particular embodiments, methods of the present invention are used to treat infections by bacteria used in biowarfare. Biowarfare and bioterrorism have been defined as the intentional or the alleged use of viruses, bacteria, fungi and toxins to produce death or disease in humans, animals or plants. Of these various biowarfare agents, bacteria and viruses appear to pose the most significant threat of widespread harm, primarily due to their relative ease of both production and transmissibility, as well as a lack of medical treatments. Examples of biowarfare bacteria and spores that may be treated according to the present invention include, but are not limited to, Bacillus anthracis, Bacillus cereus, Clostridium botulinum, Yersinia pestis, Yersinia enterocolitica, Francisella tularensis, Brucella species, Clostridium perfringens, Burkholderia mallei, Burkholderia pseudomallei, Staphylococcus species, Tuberculosis species, Escherichia coli, Group A Streptococcus, Group B Streptococcus, Streptococcus pneumoniae, Helicobacter pylori, Francisella tularensis, Salmonella enteritidis, Mycoplasma hominis, Mycoplasma orale, Mycoplasma salivarium, Mycoplasma fermentans, Mycoplasma pneumoniae, Mycobacterium bovis, Mycobacterium tuberculosis, Mycobacterium avium, Mycobacterium leprae, Rickettsia rickettsii, Rickettsia akari, Rickettsia prowazekii, Rickettsia canada, and Coxiella burnetti.

Since the methods of the present invention may be used to treat infections caused by any of a wide variety of bacteria, they may be used to treat a large number of bacterial infections and conditions, including, but not limited to, intra-abdominal infections (including complicated intra-abdominal infections), ear infections, gastrointestinal infections, bone, joint, and soft tissue infections, sinus infections, bacterial infections of the skin, bacterial infections of the lungs, urinary tract infections (UTIs), respiratory tract infections, septicemia, sinusitis, sexually transmitted diseases, ophthalmic infections, tuberculosis, pneumonia, lyme disease, hospital-acquired pneumonia (HAP), blood stream infections (BSI), peritonitis and other severe intra-abdominal infections, severe pelvic inflammatory disease, endocarditis, mycobacterium infections, neonatal sepsis, and various ocular infections, and Legionnaire's disease. Aminoglycosides are also frequently used in combination with penicillins and cephalosporins to treat both gram-positive and gram-negative bacteria.

Methods of the present invention are used, in certain embodiments, to treat any classification of urinary tract infection (UTI), and UTIs caused by any microorganism. In specific embodiments, the urinary tract infection (UTI) is a complicated UTI (cUTI) or an uncomplicated UTI (uUTI). Acute infections of the urinary tract may be categorized as either uncomplicated or complicated. Lower tract infections, including cystitis and urethritis, generally fall into the uncomplicated category. Lower tract infections, however, are considered complicated if the infection occurs in patients with any of the following: 1) indwelling catheter, 2) residual post-voiding volume, 3) neurogenic bladder, 4) evidence of obstructive uropathy, 5) azotemia due to intrinsic renal disease, or 6) urinary retention in men due to benign prostatic hypertrophy. Upper tract infections, manifested by signs and symptoms of an ascending infection, generally fall into the complicated category. Acute pyelonephritis requiring hospitalization generally falls into the complicated category because this condition often requires IV antibiotic management similar to the treatment and management of cUTI (Stamm, Urinary Tract Infections, Harrison's Principles of Internal Medicine, 15^{th} Ed., 2001, Ed Braunwald, Fauci, Kasper, Hauser, Longo, Jameson. Chp. 280:1620-1625; FDACDE, USDHHS, Guidance for Industry, Complicated Urinary Tract Infections and Pyelonephritis—Developing Antimicrobial Drugs for Treatment, DRAFT GUIDANCE, July 1998; Warren et al., Clin. Infect. Dis. 1999, 29(4):745-758).

Diagnosis of cUTI in men and women with a functional or anatomical abnormality of the urinary tract is based on documented infection in urine culture and a clinical presentation that may include any or all of the following: dysuria, frequency, urgency, fever, chills, malaise, nausea, vomiting, flank pain, back pain, and costo-vertebral angle (CVA) pain or tenderness (Stamm, Urinary Tract Infections, Harrison's Principles of Internal Medicine, 15^{th} Ed., 2001, Ed Braunwald, Fauci, Kasper, Hauser, Longo, Jameson. Chp. 280:1620-1625; FDACDE, USDHHS, Guidance for Industry, Complicated Urinary Tract Infections and Pyelonephritis—Developing Antimicrobial Drugs for Treatment, DRAFT GUIDANCE, July 1998).

The urinary tract is the leading site of nosocomial infection in patients in US hospitals (Klevens, et al., Pub. Health Rep 2007, 122:160-166). In the US, urinary tract infections account for nearly one-third (32%) of all annually reported healthcare-associated infections: over 400,000 healthcare-associated urinary tract infections occur in patients who are hospitalized but not admitted to the intensive care unit (ICU), and over 100,000 of such infections occur in ICU patients (Klevens, et al., Pub. Health Rep 2007, 122:160-166). Over 75% of nosocomial urinary tract infections, mostly cUTI and pyelonephritis, are caused by Gram-negative microorganisms, including Enterobacteriaceae and Pseudomnonas aeruginosa (Gaynes and Edwards, Clin. Inf. Dis. 2005, 41:848-854). The most commonly reported pathogen isolated from patients with UTI who are hospitalized in the ICU is Escherichia coli. In addition, the rate of K. pneumoniae reported for these patients has increased significantly between 1986 and 2004 (Gaynes and Edwards, Clin. Inf. Dis. 2005, 41:848-854). E. coli accounts for 80% or more of instances of uncomplicated pyelonephritis and is the most common microorganism isolated from patients with complicated pyelonephritis (Talan, et al., CID 2008, 47:1150-1158; Bergeron Med Clinics N. Am. 1995, 79(3):619-649). Of particular concern is the recent rise in antibiotic resistance of E. coli strains isolated from the urinary tracts of emergency room patients diagnosed with pyelonephritis. The resistance rate of these isolates to trimethoprim-sulfamethoxazole is reported at 20% or higher, and resistance to fluoroquinolones is increasing (Talan, et al., CID 2008, 47:1150-1158).

Most cases of complicated urinary tract infections originate while a patient is hospitalized, whereas most cases of acute pyelonephritis originate out of the hospital. However, most patients with acute pyelonephritis, particularly complicated cases, eventually require hospitalization (Johnson and Stamm, Ann. Intern. Med. 1989, 111:906-17). Complicated UTI and acute pyelonephritis are treated empirically with IV antibiotics such as carbapenems, piperacillin/tazobactam, and fluoroquinolones (Sanford Guide to Antimicrobial Therapy, 37^{th} ed. Gilbert et al. Eds. Antimicrobial Tehrapy: Sperryville, Va. 2007, p. 93). Because of their ability to accumulate in the renal cortex, achieve high urinary concentrations, and display bactericidal activity against common urinary tract pathogens, aminoglycosides remain an important option for IV treatment of cUTI (Bergeron, Med. Clinics N. Am. 1995, 79(3):619-649; Beauchamp and Bergeron, Current Inf. Dis. Reports 1999, 1:371-378; Sanford Guide to Antimicrobial Therapy, 37^{th} ed. Gilbert et al. Eds. Antimicrobial Tehrapy: Sperryville, Va. 2007, p. 93).

The National Institutes of Health (NIH), Kidney and Urologic Diseases Advisory Board (KUDAB), estimates that nearly 12 million cases of urinary tract infections (UTI) are reported annually in the United States (US) (Griebling, Urinary Tract Infection in womend. In: Litwin and Saigal, eds. Urological Diseases in America. DHHS, PHS, NIH, NIDDK. Washington, D.C.: GPO; 2007 NIG publication 07-5512:587-619; NCHS, National Hospital Discharge Survey: 2004 Annual Summary with Detailed Diagnosis and Procedure Data. DHHS, Centers for Disease Control and Prevention. Hyattsville, Md.: GPO; 2006. DHHS publication 2006-1733).

Enterobacteriaceae are by far the most predominant microorganism associated with acute infections of the lower urinary tract. In a meta-analysis of six well-controlled trials involving women with acute cystitis, the most common pathogen isolated from urine cultures was E. coli (78.6%), followed by Staphylococcus saprophyticus (4.4%), K. pneumoniae (4.3%), and Proteus mirabilis (3.7%) (Echols, et al., Clin. Inf. Dis. 1999, 29:113-119).

Resistance to common therapies for acute UTI is increasing. In the United States, in 2001, resistance to sulfamethoxazole increased to 17% overall among the more than 286,000 clinical isolates gathered from outpatient women with UTI during 1995 to 2001. Also in 2001, resistance to ciprofloxacin increased 3-fold, from 0.7% to 2.5% (Karlowsky, et al., Antimicrob. Agents Chemother. 2002 46(8):2540-2545). More worrisome that year was the reported higher than 12% rate of multiple-drug resistant (MDR) E. coli. (Multiple-drug resistance is defined as resistance to at least one drug from three different classes (Karlowsky, et al., Antimicrob. Agents Chemother. 2002 46(8):2540-2545].) By 2005, more than 50% of fluoroquinolone-resistant E. coli isolates from outpatient women from 40 sites in the North American Urinary Tract Infection Collaborative Alliance were shown to be resistant to at least two other classes of antibiotics (Karlowsky, et al., Antimicrob. Agents Chemother. 2006, 50(6):2251-4). In one European surveillance study, nearly 1% of the urinary isolates of E. coli from Spain were MDR, demonstrating resistance to at least seven different antibiotics (Kahlmeter and Menday, J. Antimicrob. Chemother. 2003, 52(1):128-31). Resistance to trimethoprim-sulfamethoxazole among E. coli urinary isolates from US emergency rooms is now reported at a rate of 20% or higher (Talan, et al., CID 2008, 47:1150-1158).

Most uncomplicated UTIs are treated on an outpatient basis. In a small, randomized clinical trial, a single IM dose of aminoglycoside showed efficacy comparable to a 5-day course of trimethoprim/sulfamethoxazole (Bailey, et al., NZ Med. J. 1984, 97(754):262-4); thus, aminoglycoside therapy administered as a single dose on an outpatient basis can be an attractive alternative to compounds requiring longer durations of dosing.

### C. Aminoglycosides

The methods of the present invention may be used to treat infection using any aminoglycoside. Aminoglycosides are a group of antibiotics found to be effective against a wide variety of bacteria, including gram-negative bacteria. In certain embodiments, the administered aminoglycoside is one of the aminoglycosides described herein, or an analog, stereoisomer, pharmaceutically acceptable salt or prodrug thereof.

Examples of aminoglycosides that may be used according to the methods of the present invention include, but are not limited to, 1,2′-N-DL-isoseryl-3′,4′-dideoxykanamycin B, 1,2′-N-DL-isoseryl-kanamycin B, 1,2′-N—[(S)-4-amino-2-hydroxybutyryl]-3′,4′-dideoxykanamycin B, 1,2′-N—[(S)-4-amino-2-hydroxybutyryl]kanamycin B, 1-N-(2-Aminobutanesulfonyl)kanamycin A, 1-N-(2-aminoethanesulfonyl)-3′,4′-dideoxyribostamycin, 1-N-(2-Aminoethanesulfonyl)-3′-deoxyribostamycin, 1-N-(2-aminoethanesulfonyl)-3′,4′-dideoxykanamycin B, 1-N-(2-aminoethanesulfonyl)kanamycin A, 1-N-(2-aminoethanesulfonyl)kanamycin B, 1-N-(2-aminoethanesulfonyl)ribostamycin, 1-N-(2-aminopropanesulfonyl)-3′-deoxykanamycin B, 1-N-(2-aminopropanesulfonyl)-3′,4′-dideoxykanamycin B, 1-N-(2-aminopropanesulfonyl)kanamycin A, 1-N-(2-aminopropanesulfonyl)kanamycin B, 1-N-(L-4-amino-2-hydroxy-butyryl)-2′,3′-dideoxy-2′-fluorokanamycin A, 1-N-(L-4-amino-2-hydroxy-propionyl)-2′,3′-dideoxy-2′-fluorokanamycin A, 1-N-DL-3′,4′-dideoxy-isoserylkanamycin B, 1-N-DL-isoserylkanamycin, 1-N-DL-isoserylkanamycin B, 1-N-[L-(−)-(alpha-hydroxy-gamma-aminobutyryl)]-XK-62-2,2′,3′-dideoxy-2′-fluorokanamycin A, 2-hydroxygentamycin A3, 2-hydroxygentamycin B, 2-hydroxygentamycin B1, 2-hydroxygentamycin JI-20A, 2-hydroxygentamycin JI-20B, 3″-N-methyl-4″-C-methyl-3′,4′-dodeoxy kanamycin A, 3″-N-methyl-4″-C-methyl-3′,4′-dodeoxy kanamycin B, 3″-N-methyl-4″-C-methyl-3′,4′-dodeoxy-6′-methyl kanamycin B, 3′,4′-Dideoxy-3′-eno-ribostamycin, 3′,4′-dideoxyneamine, 3′,4′-dideoxyribostamycin, 3′-deoxy-6′-N-methyl-kanamycin B, 3′-deoxyneamine, 3′-deoxyribostamycin, 3′-oxysaccharocin, 3,3′-nepotrehalosadiamine, 3-demethoxy-2″-N-formimidoylistamycin B disulfate tetrahydrate, 3-demethoxyistamycin B, 3-O-demethyl-2-N-formimidoylistamycin B, 3-O-demethylistamycin B, 3-trehalosamine, 4″,6″-dideoxydibekacin, 4-N-glycyl-KA-6606VI, 5″-Amino-3′,4′,5″-trideoxy-butirosin A, 6″-deoxydibekacin, 6′-epifortimicin A, 6-deoxy-neomycin (structure 6-deoxy-neomycin B), 6-deoxy-neomycin B, 6-deoxy-neomycin C, 6-deoxy-paromomycin, acmimycin, AHB-3′,4′-dideoxyribostamycin, AHB-3′-deoxykanamycin B, AHB-3′-deoxyneamine, AHB-3′-deoxyribostamycin, AHB-4″-6″-dideoxydibekacin, AHB-6″-deoxydibekacin, AHB-dideoxyneamine, AHB-kanamycin B, AHB-methyl-3′-deoxykanamycin B, amikacin, amikacin sulfate, apramycin, arbekacin, astromicin, astromicin sulfate, bekanamycin, bluensomycin, boholmycin, butirosin, butirosin B, catenulin, coumamidine gamma1, coumamidine gamma2,D,L-1-N-(alpha-hydroxy-beta-aminopropionyl)-XK-62-2, dactimicin, de-O-methyl-4-N-glycyl-KA-6606VI, de-O-methyl-KA-6606I, de-O-methyl-KA-7038I, destomycin A, destomycin B, di-N6′,O3-demethylistamycin A, dibekacin, dibekacin sulfate, dihydrostreptomycin, dihydrostreptomycin sulfate, epi-formamidoylglycidylfortimicin B, epihygromycin, formimidoyl-istamycin A, formimidoyl-istamycin B, fortimicin B, fortimicin C, fortimicin D, fortimicin KE, fortimicin KF, fortimicin KG, fortimicin KG1 (stereoisomer KG1/KG2), fortimicin KG2 (stereoisomer KG1/KG2), fortimicin KG3, framycetin, framycetin sulphate, gentamicin, gentamycin sulfate, globeomycin, hybrimycin A1, hybrimycin A2, hybrimycin B1, hybrimycin B2, hybrimycin C1, hybrimycin C2, hydroxystreptomycin, hygromycin, hygromycin B, isepamicin, isepamicin sulfate, istamycin, kanamycin, kanamycin sulphate, kasugamycin, lividomycin, marcomycin, micronomicin, micronomicin sulfate, mutamicin, myomycin, N-demethyl-7-O-demethylcelesticetin, demethylcelesticetin, methanesulfonic acid derivative of istamycin, nebramycin, nebramycin, neomycin, netilmicin, netromycin, oligostatin, paromomycin, quintomycin, ribostamycin, saccharocin, seldomycin, sisomicin, sorbistin, spectinomycin, streptomycin, tobramycin, trehalosmaine, trestatin, validamycin, verdamycin, xylostasin, zygomycin and analogs, stereoisomers, pharmaceutically acceptable salts and prodrugs thereof. In particular embodiments, the aminoglycoside is amikacin, gentamycin, tobramycin, netromycin, apramycin, streptomycin, kanamycin, dibekacin, arbekacin, paromomycin, neomycin, netilmicin, sisomicin, or an analog, stereoisomer, pharmaceutically acceptable salt or prodrug thereof. In particular embodiments, the aminoglycoside is gentamicin or amikacin.

In certain embodiments, the adminstered aminoglycoside is amikacin, gentamicin, tobramycin, netromycin, apramycin, streptomycin, kanamycin, dibekacin, arbekacin, paromomycin, neomycin, netilmicin, or sisomicin, or an analog, stereoisomer, pharmaceutically acceptable salt or prodrug thereof. In related embodiments, the administered aminoglycoside is sisomycin, amikacin, kanamycin, arbekacin, dibekacin, tobramycin, neomycin, or gentamicin, or an analog, stereoisomer, pharmaceutically acceptable salt or prodrug thereof. In particular embodiments, the aminoglycoside is sisomicin, gentamicin, amikacin, or neomycin, or an analog, stereoisomer, pharmaceutically acceptable salt or prodrug thereof. In one embodiment, the aminoglycoside is sisomicin, or an analog, stereoisomer, pharmaceutically acceptable salt or prodrug thereof. In one embodiment, the aminoglycoside is gentamicin, or an analog, stereoisomer, pharmaceutically acceptable salt or prodrug thereof. In one embodiment, the aminoglycoside is amikacin, or an analog, stereoisomer, pharmaceutically acceptable salt or prodrug thereof. In one embodiment, the aminoglycoside is neomycin, or an analog, stereoisomer, pharmaceutically acceptable salt or prodrug thereof.

Aminoglycosides may be synthesized as known in the art or described herein. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, e.g., Advanced Organic Chemistry: Reactions, Mechanisms, and Structure, 5th edition (Wiley, December 2000)) or prepared as described herein. It is understood that one skilled in the art may be able to make these compounds by similar methods or by combining other methods known to one skilled in the art. It is also understood that one skilled in the art would be able to make, in a similar manner as described below, other compounds of structures (A)—(F) and (I)-(VIII) not specifically illustrated below by using the appropriate starting components and modifying the parameters of the synthesis as needed. The synthetic methods provided herein are for purposes of illustration, not limitation.

As used herein, unless specified to the contrary, the following terms have the meaning indicated.

“Amino” refers to the —NH₂ radical.

“Cyano” refers to the —CN radical.

“Hydroxy” or “hydroxyl” refers to the —OH radical.

“Imino” refers to the ═NH substituent.

“Nitro” refers to the —NO₂ radical.

“Oxo” refers to the ═O substituent.

“Thioxo” refers to the ═S substituent.

“Alkyl” refers to a straight or branched hydrocarbon chain radical consisting solely of carbon and hydrogen atoms, which is saturated or unsaturated (i.e., contains one or more double and/or triple bonds), having from one to twelve carbon atoms (C₁-C₁₂ alkyl), preferably one to eight carbon atoms (C₁-C₈ alkyl) or one to six carbon atoms (C₁-C₆ alkyl), and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, ethenyl, prop-1-enyl, but-1-enyl, pent-1-enyl, penta-1,4-dienyl, ethynyl, propynyl, butynyl, pentynyl, hexynyl, and the like. Unless stated otherwise specifically in the specification, an alkyl group may be optionally substituted.

“Alkylene” or “alkylene chain” refers to a straight or branched divalent hydrocarbon chain linking the rest of the molecule to a radical group, consisting solely of carbon and hydrogen, which is saturated or unsaturated (i.e., contains one or more double and/or triple bonds), and having from one to twelve carbon atoms, e.g., methylene, ethylene, propylene, n-butylene, ethenylene, propenylene, n-butenylene, propynylene, n-butynylene, and the like. The alkylene chain is attached to the rest of the molecule through a single or double bond and to the radical group through a single or double bond. The points of attachment of the alkylene chain to the rest of the molecule and to the radical group can be through one carbon or any two carbons within the chain. Unless stated otherwise specifically in the specification, an alkylene chain may be optionally substituted.

“Alkoxy” refers to a radical of the formula —ORₐ, where Rₐ is an alkyl radical as defined above containing one to twelve carbon atoms. Unless stated otherwise specifically in the specification, an alkoxy group may be optionally substituted.

“Alkylamino” refers to a radical of the formula —NHRₐ or —NRₐRₐ where each Rₐ is, independently, an alkyl radical as defined above containing one to twelve carbon atoms. Unless stated otherwise specifically in the specification, an alkylamino group may be optionally substituted.

“Thioalkyl” refers to a radical of the formula —SRₐ, —SORₐ or —SO₂Rₐ where Rₐ is an alkyl radical as defined above containing one to twelve carbon atoms. Unless stated otherwise specifically in the specification, a thioalkyl group may be optionally substituted.

“Aryl” refers to a hydrocarbon ring system radical comprising hydrogen, 6 to 18 carbon atoms and at least one aromatic ring. For purposes of this invention, the aryl radical may be a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

“Aralkyl” refers to a radical of the formula —R_{b}-R_{c} where R_{b} is an alkylene chain as defined above and R_{c} is one or more aryl radicals as defined above, for example, benzyl, diphenylmethyl and the like. Unless stated otherwise specifically in the specification, an aralkyl group may be optionally substituted.

“Cycloalkyl” or “carbocyclic ring” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group may be optionally substituted.

“Cycloalkylalkyl” refers to a radical of the formula —R_{b}R_{d} where R_{d} is an alkylene chain as defined above and R_{g} is a cycloalkyl radical as defined above. Unless stated otherwise specifically in the specification, a cycloalkylalkyl group may be optionally substituted.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Heterocyclyl” or “heterocyclic ring” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, Unless stated otherwise specifically in the specification, a heterocyclyl group may be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group may be optionally substituted.

“Heterocyclylalkyl” refers to a radical of the formula —R_{b}Rₑ where R_{b} is an alkylene chain as defined above and Rₑ is a heterocyclyl radical as defined above, and if the heterocyclyl is a nitrogen-containing heterocyclyl, the heterocyclyl may be attached to the alkyl radical at the nitrogen atom. Unless stated otherwise specifically in the specification, a heterocyclylalkyl group may be optionally substituted.

“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group may be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group may be optionally substituted.

“Heteroarylalkyl” refers to a radical of the formula —R_{b}R_{f} where R_{b} is an alkylene chain as defined above and R_{f} is a heteroaryl radical as defined above. Unless stated otherwise specifically in the specification, a heteroarylalkyl group may be optionally substituted.

The term “substituted” used herein means any of the above groups (i.e., alkyl, alkylene, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles.

For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}C(═NR_{g})NR_{g}Rₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl group. In addition, each of the foregoing substituents may also be optionally substituted with one or more of the above substituents.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

The compounds used according to the invention, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. The present invention includes tautomers of any said compounds.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of a compound. As used herein, the term “solvate” refers to an aggregate that comprises one or more molecules of a compound with one or more molecules of solvent. The solvent may be water, in which case the solvate may be a hydrate. Alternatively, the solvent may be an organic solvent. Thus, compounds may exist as a hydrate, including a monohydrate, dihydrate, hemihydrate, sesquihydrate, trihydrate, tetrahydrate and the like, as well as the corresponding solvated forms. Compounds may be true solvates, while in other cases, compounds may merely retain adventitious water or be a mixture of water plus some adventitious solvent.

“Prodrug” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., A.C.S. Symposium Series, Vol. 14, and in Bioreversible Carriers in Drug Design, Ed. Edward B. Roche, American Pharmaceutical Association and Pergamon Press, 1987.

The term “prodrug” is also meant to include any covalently bonded carriers, which release the active compound of the invention in vivo when such prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention. Prodrugs include compounds of the invention wherein a hydroxyl, amino or mercapto group is bonded to any group that, when the prodrug of the compound of the invention is administered to a mammalian subject, cleaves to form a free hydroxyl, free amino or free mercapto group, respectively. Examples of prodrugs include, but are not limited to, acetate, formate and benzoate derivatives of alcohol or amide derivatives of amine functional groups in the compounds of the invention and the like.

The phrases “or an analog, stereoisomer, pharmaceutically acceptable salt or prodrug thereof” and “or a stereoisomer, pharmaceutically acceptable salt of prodrug thereof” is meant to include all combinations thereof, for example: stereoisomers, pharmaceutically acceptable salts and prodrugs of analogs; analogs, stereoisomers and pharmaceutically acceptable salts of prodrugs; analogs, pharmaceutically acceptable salts and prodrugs of stereoisomers; and analogs, stereoisomers and produgs of pharmaceutically acceptable salts.

1. Sisomicin and Analogs Thereof

Methods of the present invention may be practiced using sisomicin, or an analog, stereoisomer, pharmaceutically acceptable salt or prodrug thereof, including but not limited to those described herein.

In one embodiment of the present invention, methods are practiced using sisomicin.

In one embodiment of the present invention, methods are practiced using sisomicin analogs, or compounds, having the following structure (A):

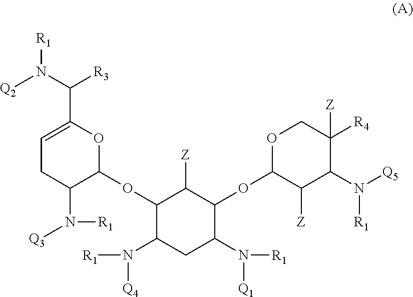

or a stereoisomer, pharmaceutically acceptable salt or prodrug thereof,

wherein:

Q₁, Q₂, Q₃, Q4 and Q₅ are, independently, hydrogen, optionally substituted alkyl, optionally substituted thioalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, or optionally substituted heteroarylalkyl;

each Z is, independently, halogen or —OR₂

each R₁ is, independently, hydrogen or an amino protecting group;

each R₂ is, independently, hydrogen or a hydroxyl protecting group;

R₃ is hydrogen or C₁-C₆ alkyl; and

R₄ is hydrogen or methyl.

In one embodiment of the present invention, methods are practiced using sisomicin analogs, or compounds, having the following structure (I):

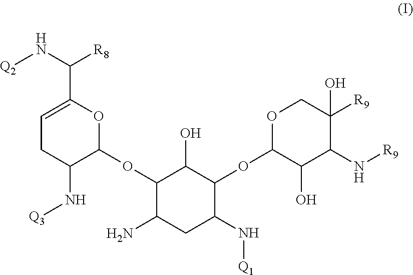

or a stereoisomer, pharmaceutically acceptable salt or prodrug thereof,

wherein:

Q₁ is hydrogen,

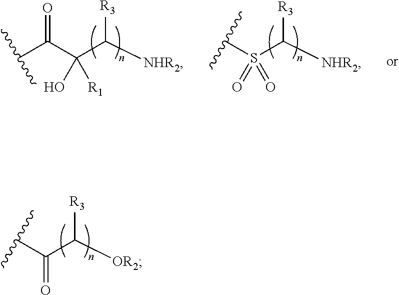

Q₂ is hydrogen, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₄R₅, —(CR₁₀R₁₁)ₚR₁₂,

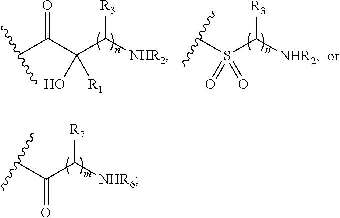

Q₃ is hydrogen, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₄R₅, —(CR₁₀R₁₁)ₚR₁₂,

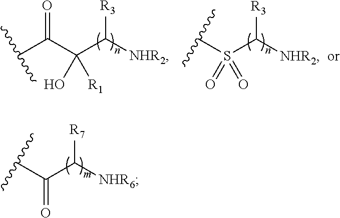

each R₁, R₂, R₃, R₄, R₅, R₈ and R₁₀ is, independently, hydrogen or C₁-C₆ alkyl, or R₁ and R₂ together with the atoms to which they are attached can form a heterocyclic ring having from 4 to 6 ring atoms, or R₂ and R₃ together with the atoms to which they are attached can form a heterocyclic ring having from 4 to 6 ring atoms, or R₁ and R₃ together with the atoms to which they are attached can form a carbocyclic ring having from 4 to 6 ring atoms, or R₄ and R₅ together with the atom to which they are attached can form a heterocyclic ring having from 4 to 6 ring atoms;

each R₆ and R₇ is, independently, hydrogen, hydroxyl, amino or C₁-C₆ alkyl, or R₆ and R₇ together with the atoms to which they are attached can form a heterocyclic ring having from 4 to 6 ring atoms;

each R₉ is, independently, hydrogen or methyl;

each R₁₁ is, independently, hydrogen, hydroxyl, amino or C₁-C₆ alkyl;

each R₁₂ is, independently, hydroxyl or amino;

each n is, independently, an integer from 0 to 4;

each m is, independently, an integer from 0 to 4; and

each p is, independently, an integer from 1 to 5, and

wherein (i) at least two of Q₁, Q₂ and Q₃ are other than hydrogen, and (ii) if Q₁ is hydrogen, then at least one of Q₂ and Q₃ is —C(═NH)NR₄R₅.

In further embodiments, R₈ is hydrogen.

In further embodiments, each R₉ is methyl.

In further embodiments, Q₁ and Q₂ are other than hydrogen. In certain embodiments of the foregoing, Q₃ is hydrogen.

In more specific embodiments of the foregoing, Q₁ is:

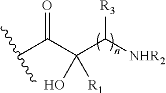

wherein: R₁ is hydrogen; R₂ is hydrogen; and each R₃ is hydrogen. For example, Q₁ may be:

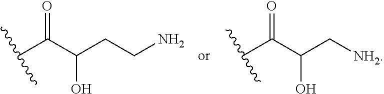

In other more specific embodiments of the foregoing, Q₁ is:

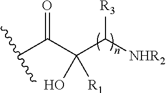

wherein: R₁ is hydrogen; and R₂ and R₃ together with the atoms to which they are attached form a heterocyclic ring having from 4 to 6 ring atoms. For example, Q₁ may be:

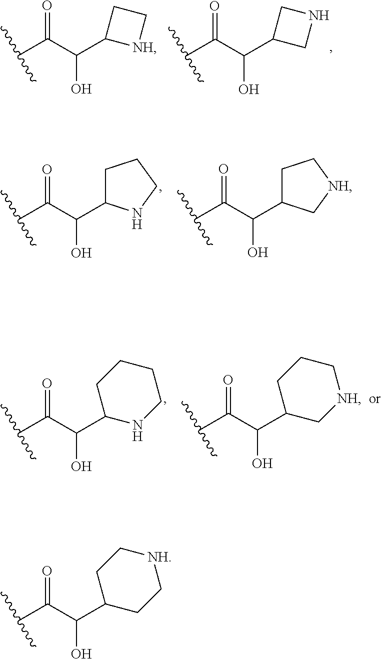

In other more specific embodiments of the foregoing, Q₁ is:

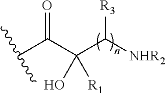

wherein: R₃ is hydrogen; and R₁ and R₂ together with the atoms to which they are attached form a heterocyclic ring having from 4 to 6 ring atoms. For example, Q₁ may be:

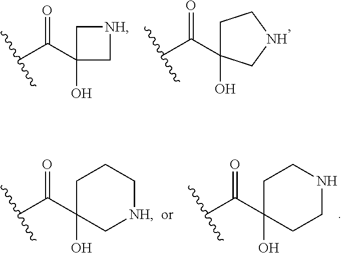

In other more specific embodiments of the foregoing, Q₁ is:

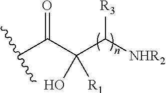

wherein: R₂ is hydrogen; and R₁ and R₃ together with the atoms to which they are attached form a carbocyclic ring having from 4 to 6 ring atoms. For example, Q₁ may be:

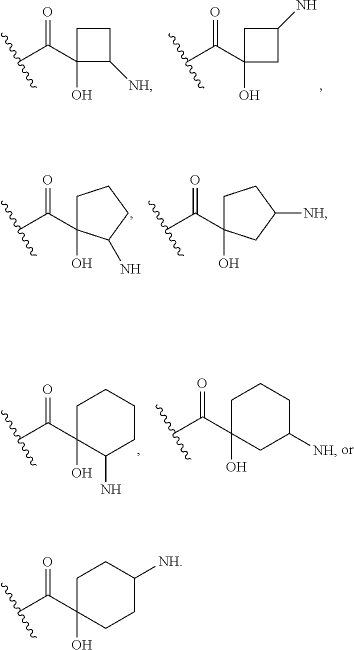

In other more specific embodiments of the foregoing, Q₁ is:

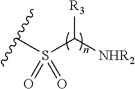

wherein: R₂ is hydrogen; and each R₃ is hydrogen.

In other more specific embodiments of the foregoing, Q₁ is:

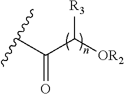

wherein: R₂ is hydrogen; and each R₃ is hydrogen.

In other more specific embodiments of the foregoing, Q₂ is —(CR₁₀R₁₁)ₚR₁₂. In certain embodments, each R₁₀ is hydrogen. In certain embodiments, each R₁₁ is hydrogen.

In other more specific embodiments of the foregoing, Q₂ is optionally substituted cycloalkylalkyl. In certain embodiments, Q₂ is unsubstituted. In certain embodiments, Q₂ is substituted with hydroxyl or amino.

In other more specific embodiments of the foregoing, Q₂ is optionally substituted heterocyclylalkyl. In certain embodiments, Q₂ is unsubstituted. In certain embodiments, Q₂ is substituted with hydroxyl or amino.

In other further embodiments, Q₁ and Q₃ are other than hydrogen. In certain embodiments, Q₂ is hydrogen.

In more specific embodiments of the foregoing, Q₁ is:

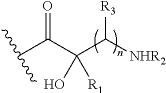

wherein: R₁ is hydrogen; R₂ is hydrogen; and each R₃ is hydrogen. For example, Q₁ may be:

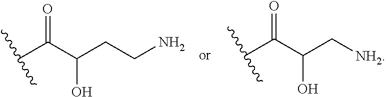

In other more specific embodiments of the foregoing, Q₁ is:

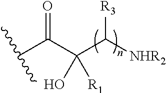

wherein:

R₁ is hydrogen; and

R₂ and R₃ together with the atoms to which they are attached form a heterocyclic ring having from 4 to 6 ring atoms. For example, Q₁ may be:

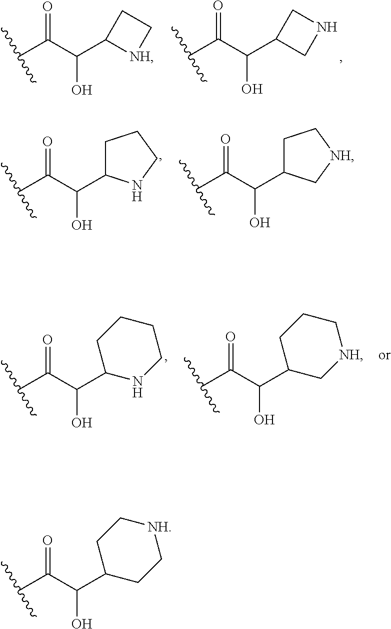

In other more specific embodiments of the foregoing, Q₁ is:

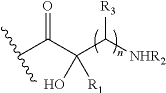

wherein: R₃ is hydrogen; and R₁ and R₂ together with the atoms to which they are attached form a heterocyclic ring having from 4 to 6 ring atoms. For example, Q₁ may be:

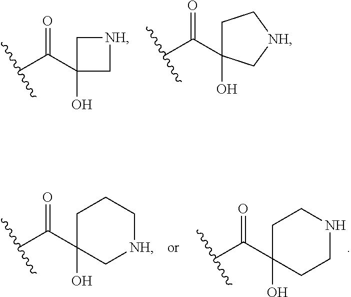

In other more specific embodiments of the foregoing, Q₁ is:

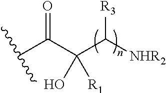

wherein: R₂ is hydrogen; and R₁ and R₃ together with the atoms to which they are attached form a carbocyclic ring having from 4 to 6 ring atoms. For example, Q₁ may be:

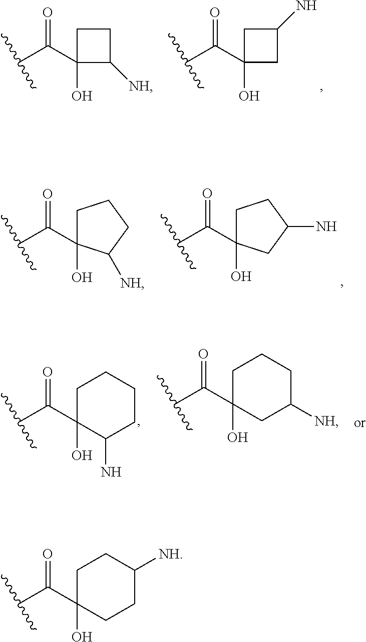

In other more specific embodiments of the foregoing, Q₁ is:

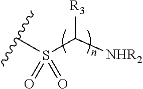

wherein: R₂ is hydrogen; and each R₃ is hydrogen.

In other more specific embodiments of the foregoing, Q₁ is:

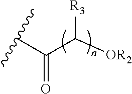

wherein: R₂ is hydrogen; and each R₃ is hydrogen.

In other more specific embodiments of the foregoing, Q₃ is —(CR₁₀R₁₁)ₚR₁₂. In certain embodments, each R₁₀ is hydrogen. In certain embodiments, each R₁₁ is hydrogen.

In other more specific embodiments of the foregoing, Q₃ is optionally substituted cycloalkylalkyl. In certain embodiments, Q₃ is unsubstituted. In certain embodiments, Q₃ is substituted with hydroxyl or amino.

In other more specific embodiments of the foregoing, Q₃ is optionally substituted heterocyclylalkyl. In certain embodiments, Q₃ is unsubstituted. In certain embodiments, Q₃ is substituted with hydroxyl or amino.

In other more specific embodiments of the foregoing, Q₃ is optionally substituted heterocyclyl. In certain embodiments, Q₃ is unsubstituted. In certain embodiments, Q₃ is substituted with hydroxyl or amino.

In other more specific embodiments of the foregoing, Q₃ is —C(═NH)NH₂.

In other further embodiments, Q₂ and Q₃ are other than hydrogen. In certain embodiments, Q₁ is hydrogen.

In more specific embodiments of the foregoing, Q₂ is —C(═NH)NH₂.

In other more specific embodiments of the foregoing, Q₃ is —C(═NH)NH₂.

In one embodiment of the present invention, methods are practiced using sisomicin analogs, or compounds, having the following structure (II):

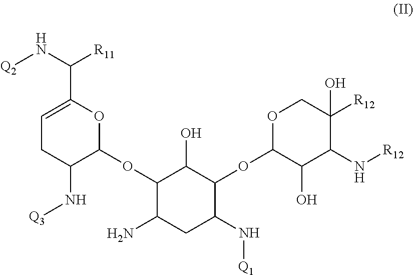

or a stereoisomer, pharmaceutically acceptable salt or prodrug thereof,

wherein:

Q₁ is alkyl optionally substituted with hydroxyl or amino,

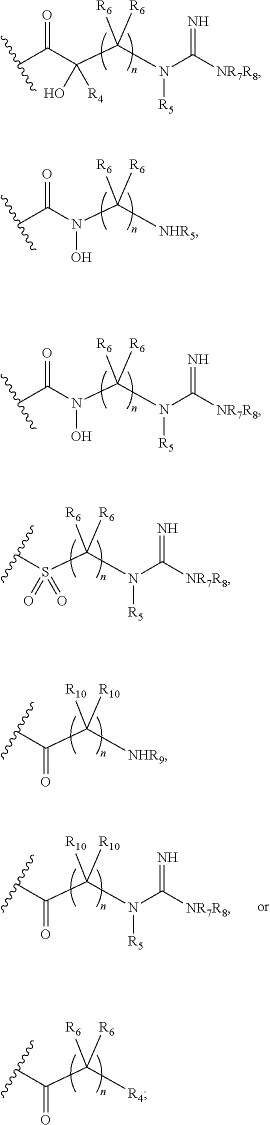

Q₂ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

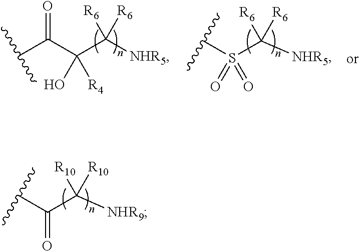

Q₃ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

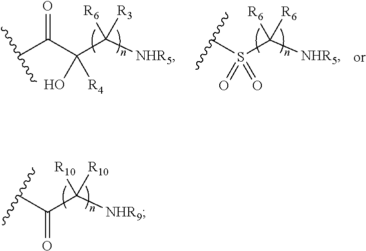

each R₄, R₅, R₇, R₈ and R₁₁ is, independently, hydrogen or C₁-C₆ alkyl optionally substituted with one or more halogen, hydroxyl or amino;

each R₆ is, independently, hydrogen, halogen, hydroxyl, amino or C₁-C₆ alkyl;

or R₄ and R₅ together with the atoms to which they are attached can form a heterocyclic ring having from 4 to 6 ring atoms, or R₅ and one R₆ together with the atoms to which they are attached can form a heterocyclic ring having from 3 to 6 ring atoms, or R₄ and one R₆ together with the atoms to which they are attached can form a carbocyclic ring having from 3 to 6 ring atoms, or R₇ and R₈ together with the atom to which they are attached can form a heterocyclic ring having from 3 to 6 ring atoms;

each R₉ and R₁₂ is, independently, hydrogen, hydroxyl, amino or C₁-C₈ alkyl optionally substituted with one or more halogen, hydroxyl or amino;

each R₁₀ is, independently, hydrogen, halogen, hydroxyl, amino or C₁-C₆ alkyl;

or R₉ and one R₁₀ together with the atoms to which they are attached can form a heterocyclic ring having from 3 to 6 ring atoms; and

each n is, independently, an integer from 0 to 4, and

wherein (i) at least one of Q₂ and Q₃ is other than hydrogen, and (ii) Q₁ is not ethyl or —C(═O)CH₃.

In further embodiments:

Q₁ is alkyl optionally substituted with hydroxyl or amino, —C(═O)H,

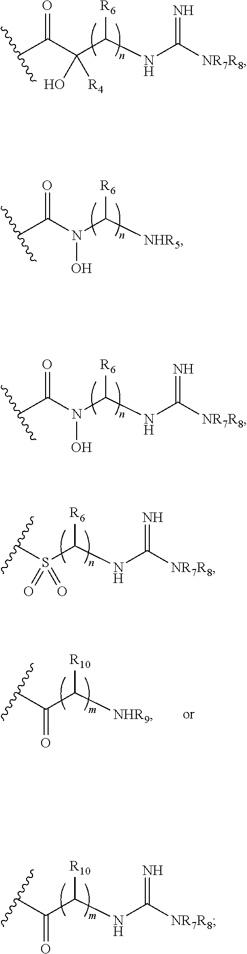

Q₂ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

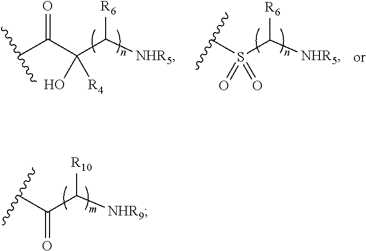

Q₃ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

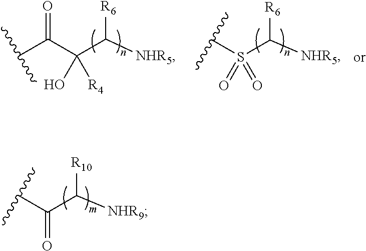

each R₄, R₅, R₆, R₇, R₈ and R₁₁ is, independently, hydrogen or C₁-C₆ alkyl, or R₄ and R₅ together with the atoms to which they are attached can form a heterocyclic ring having from 4 to 6 ring atoms, or R₅ and R₆ together with the atoms to which they are attached can form a heterocyclic ring having from 4 to 6 ring atoms, or R₄ and R₆ together with the atoms to which they are attached can form a carbocyclic ring having from 3 to 6 ring atoms, or R₇ and R₈ together with the atom to which they are attached can form a heterocyclic ring having from 4 to 6 ring atoms;

each R₉, R₁₀ and R₁₂ is, independently, hydrogen, hydroxyl, amino or C₁-C₆ alkyl, or R₉ and R₁₀ together with the atoms to which they are attached can form a heterocyclic ring having from 4 to 6 ring atoms;

each n is, independently, an integer from 0 to 4; and

each m is, independently, an integer from 0 to 4, and

wherein (i) at least one of Q₂ and Q₃ is other than hydrogen, and (ii) Q₁ is not ethyl.

In other further embodiments, Q₁ is:

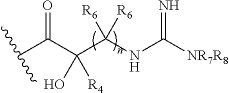

wherein: R₄ is hydrogen; R₇ is hydrogen; R₈ is hydrogen; and n is an integer from 1 to 4. In further embodiments, each R₆ is hydrogen. For example, in more specific embodiments of the foregoing, Q₁ is:

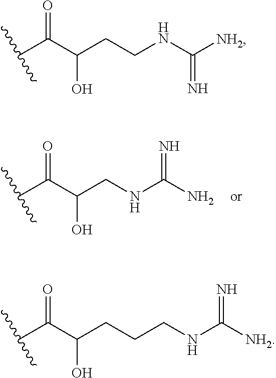

In other further embodiments, at least one R₆ is halogen.

In other further embodiments, Q₁ is:

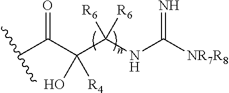

wherein: R₄ and one R₆ together with the atoms to which they are attached form a carbocyclic ring having from 3 to 6 ring atoms; R₇ is hydrogen; R₈ is hydrogen; and n is an integer from 1 to 4. For example, in more specific embodiments of the foregoing, Q₁ is:

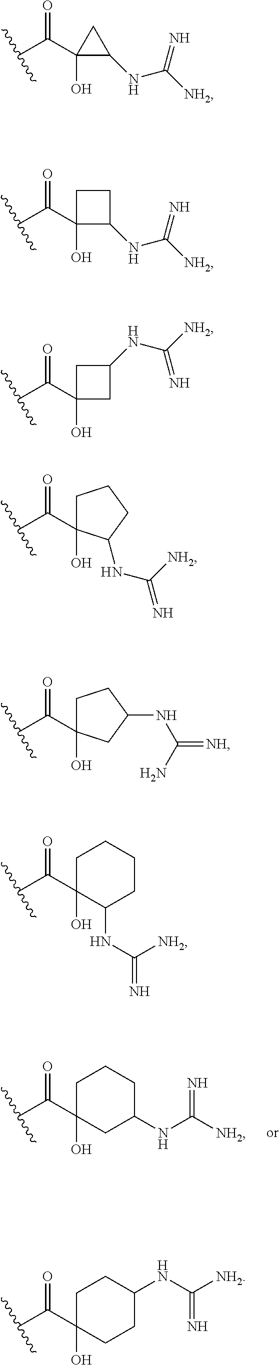

In other further embodiments, at least one R₆ is halogen.

In other further embodiments, Q₁ is:

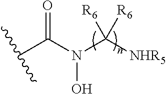

wherein R₅ is hydrogen. In further embodiments, each R₆ is hydrogen. For example, in more specific embodiments of the foregoing, Q₁ is:

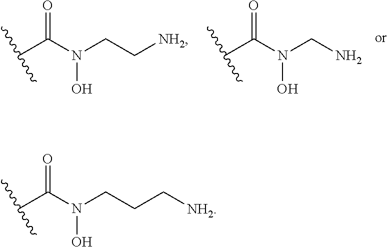

In other further embodiments, at least one R₆ is halogen.

In other further embodiments, Q₁ is:

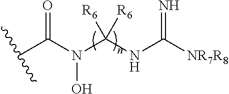

wherein: R₇ is hydrogen; and R₈ is hydrogen. In further embodiments, each R₆ is hydrogen. For example, in more specific embodiments of the foregoing, Q₁ is:

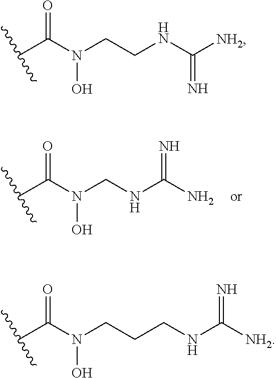

In other further embodiments, at least one R₆ is halogen.

In other further embodiments, Q₁ is:

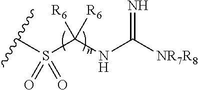

wherein: R₇ is hydrogen; and R₈ is hydrogen. In further embodiments, each R₆ is hydrogen. In other further embodiments, at least one R₆ is halogen.

In other further embodiments, Q₁ is:

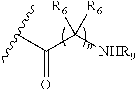

wherein R₉ is hydrogen. In further embodiments, each R₁₀ is hydrogen. In other further embodiments, at least one R₁₀ is halogen.

In other further embodiments, Q₁ is:

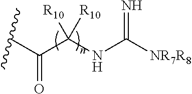

wherein: R₇ is hydrogen; and R₈ is hydrogen. In further embodiments, each R₁₀ is hydrogen. In other further embodiments, at least one R₁₀ is halogen.

In other further embodiments, Q₁ is:

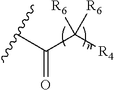

wherein R₄ is hydrogen. In further embodiments, each R₆ is hydrogen. In other further embodiments, at least one R₆ is halogen. For example, in more specific embodiments of the foregoing, Q₁ is —C(═O)H.

In other further embodiments, Q₁ is alkyl optionally substituted with hydroxyl or amino. For example, in more specific embodiments, Q₁ is unsubtituted. In other more specific embodiments, Q₁ is substituted with hydroxyl or amino.

In other further embodiments, Q₂ is other than hydrogen.

In other further embodiments, Q₂ is optionally substituted alkyl. For example, in more specific embodiments, Q₂ is unsubstituted. In other more specific embodiments, Q₂ is substituted with hydroxyl or amino.

In other further embodiments, Q₂ is optionally substituted cycloalkyl. For example, in more specific embodiments, Q₂ is unsubstituted. In other more specific embodiments, Q₂ is substituted with hydroxyl or amino.

In other further embodiments, Q₂ is optionally substituted cycloalkylalkyl. For example, in more specific embodiments, Q₂ is unsubstituted. In other more specific embodiments, Q₂ is substituted with hydroxyl or amino.

In other further embodiments, Q₂ is optionally substituted heterocyclyl. For example, in more specific embodiments, Q₂ is unsubstituted. In other more specific embodiments, Q₂ is substituted with hydroxyl or amino.

In other further embodiments, Q₂ is optionally substituted heterocyclylalkyl. For example, in more specific embodiments, Q₂ is unsubstituted. In other more specific embodiments, Q₂ is substituted with hydroxyl or amino.

In other further embodiments, Q₂ is hydrogen.

In other further embodiments, Q₃ is other than hydrogen.

In other further embodiments, Q₃ is optionally substituted alkyl. For example, in more specific embodiments, Q₃ is unsubstituted. In other more specific embodiments, Q₃ is substituted with hydroxyl or amino.

In other further embodiments, Q₃ is optionally substituted cycloalkyl.

For example, in more specific embodiments, Q₃ is unsubstituted. In other more specific embodiments, Q₃ is substituted with hydroxyl or amino.

In other further embodiments, Q₃ is optionally substituted cycloalkylalkyl. For example, in more specific embodiments, Q₃ is unsubstituted. In other more specific embodiments, Q₃ is substituted with hydroxyl or amino.

In other further embodiments, Q₃ is optionally substituted heterocyclyl. For example, in more specific embodiments, Q₃ is unsubstituted. In other more specific embodiments, Q₃ is substituted with hydroxyl or amino.

In other further embodiments, Q₃ is optionally substituted heterocyclylalkyl. For example, in more specific embodiments, Q₃ is unsubstituted. In other more specific embodiments, Q₃ is substituted with hydroxyl or amino.

In other further embodiments, Q₃ is —C(═NH)NH₂.

In other further embodiments, Q₃ is hydrogen.

In other further embodiments, R₁₁ is hydrogen.

In other further embodiments, each R₁₂ is methyl.

In one embodiment of the present invention, methods are practiced using sisomicin analogs, or compounds, having the following structure (II):

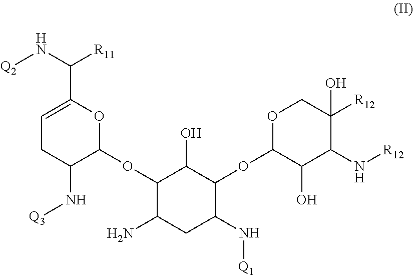

or a stereoisomer, pharmaceutically acceptable salt or prodrug thereof,

wherein:

Q₁ is

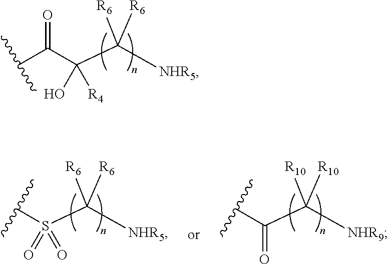

Q₂ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

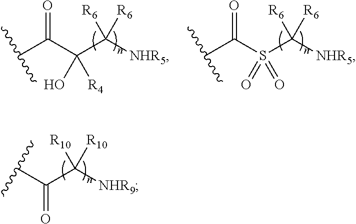

Q₃ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

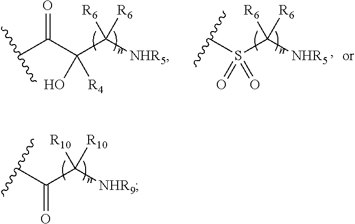

each R₄, R₅, R₇, R₈ and R₁₁ is, independently, hydrogen or C₁-C₆ alkyl optionally substituted with one or more halogen, hydroxyl or amino;

each R₆ is, independently, hydrogen, halogen, hydroxyl, amino or C₁-C₆ alkyl;

or R₄ and R₅ together with the atoms to which they are attached can form a heterocyclic ring having from 4 to 6 ring atoms, or R₅ and one R₆ together with the atoms to which they are attached can form a heterocyclic ring having from 3 to 6 ring atoms, or R₄ and one R₆ together with the atoms to which they are attached can form a carbocyclic ring having from 3 to 6 ring atoms, or R₇ and R₈ together with the atom to which they are attached can form a heterocyclic ring having from 3 to 6 ring atoms;

each R₉ and R₁₂ is, independently, hydrogen, hydroxyl, amino or C₁-C₆ alkyl optionally substituted with one or more halogen, hydroxyl or amino;

each R₁₀ is, independently, hydrogen, halogen, hydroxyl, amino or C₁-C₆ alkyl;

or R₉ and one R₁₀ together with the atoms to which they are attached can form a heterocyclic ring having from 3 to 6 ring atoms; and

each n is, independently, an integer from 0 to 4, and

wherein (i) at least one of Q₂ and Q₃ is other than hydrogen, and (ii) for Q₁, R₅ and one R₆ together with the atoms to which they are attached form a heterocyclic ring having 3 ring atoms, or R₄ and one R₆ together with the atoms to which they are attached form a carbocyclic ring having 3 ring atoms, or R₉ and one R₁₀ together with the atoms to which they are attached form a heterocyclic ring having 3 ring atoms.

In further embodiments,

Q₁ is:

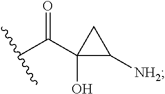

Q₂ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

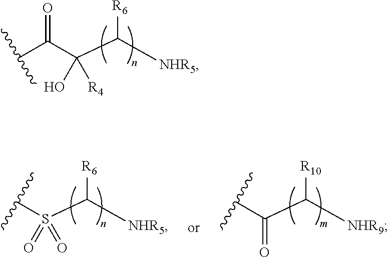

Q₃ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

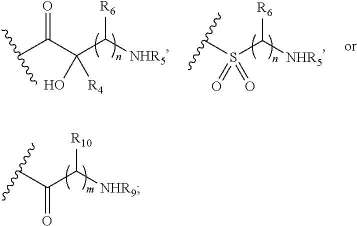

each R₄, R₅, R₆, R₇, R₈ and R₁₁ is, independently, hydrogen or C₁-C₆ alkyl, or R₄ and R₅ together with the atoms to which they are attached can form a heterocyclic ring having from 4 to 6 ring atoms, or R₅ and R₆ together with the atoms to which they are attached can form a heterocyclic ring having from 4 to 6 ring atoms, or R₄ and R₆ together with the atoms to which they are attached can form a carbocyclic ring having from 3 to 6 ring atoms, or R₇ and R₈ together with the atom to which they are attached can form a heterocyclic ring having from 4 to 6 ring atoms;

each R₉, R₁₀ and R₁₂ is, independently, hydrogen, hydroxyl, amino or C₁-C₆ alkyl, or R₉ and R₁₀ together with the atoms to which they are attached can form a heterocyclic ring having from 4 to 6 ring atoms;

each n is, independently, an integer from 0 to 4; and

each m is, independently, an integer from 0 to 4, and

wherein at least one of Q₂ and Q₃ is other than hydrogen.

In other further embodiments, Q₁ is:

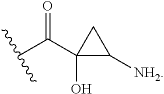

In other further embodiments, Q₂ is other than hydrogen.

In other further embodiments, Q₂ is optionally substituted alkyl. For example, in more specific embodiments, Q₂ is unsubstituted. In other more specific embodiments, Q₂ is substituted with hydroxyl or amino.

In other further embodiments, Q₂ is optionally substituted cycloalkyl. For example, in more specific embodiments, Q₂ is unsubstituted. In other more specific embodiments, Q₂ is substituted with hydroxyl or amino.

In other further embodiments, Q₂ is optionally substituted cycloalkylalkyl. For example, in more specific embodiments, Q₂ is unsubstituted. In other more specific embodiments, Q₂ is substituted with hydroxyl or amino.

In other further embodiments, Q₂ is optionally substituted heterocyclyl. For example, in more specific embodiments, Q₂ is unsubstituted. In other more specific embodiments, Q₂ is substituted with hydroxyl or amino.

In other further embodiments, Q₂ is optionally substituted heterocyclylalkyl. For example, in more specific embodiments, Q₂ is unsubstituted. In other more specific embodiments, Q₂ is substituted with hydroxyl or amino.

In other further embodiments, Q₂ is hydrogen.

In other further embodiments, Q₃ is other than hydrogen.

In other further embodiments, Q₃ is optionally substituted alkyl. For example, in more specific embodiments, Q₃ is unsubstituted. In other more specific embodiments, Q₃ is substituted with hydroxyl or amino.

In other further embodiments, Q₃ is optionally substituted cycloalkyl. For example, in more specific embodiments, Q₃ is unsubstituted. In other more specific embodiments, Q₃ is substituted with hydroxyl or amino.

In other further embodiments, Q₃ is optionally substituted cycloalkylalkyl. For example, in more specific embodiments, Q₃ is unsubstituted. In other more specific embodiments, Q₃ is substituted with hydroxyl or amino.

In other further embodiments, Q₃ is optionally substituted heterocyclyl. For example, in more specific embodiments, Q₃ is unsubstituted. In other more specific embodiments, Q₃ is substituted with hydroxyl or amino.

In other further embodiments, Q₃ is optionally substituted heterocyclylalkyl. For example, in more specific embodiments, Q₃ is unsubstituted. In other more specific embodiments, Q₃ is substituted with hydroxyl or amino.

In other further embodiments, Q₃ is —C(═NH)NH₂.

In other further embodiments, Q₃ is hydrogen.

In other further embodiments, R₁₁ is hydrogen.

In other further embodiments, each R₁₂ is methyl.

It is understood that any embodiment of the compounds of structure (I) or (II), as set forth above, and any specific substituent set forth herein for a Q₁, Q₂, Q₃, R₁, R₂, R₃, R₄, R₅, R₆, R₇, R₈, R₉, R₁₀, R₁₁ or R₁₂ group in the compounds of structure (I) or (II), as set forth above, may be independently combined with other embodiments and/or substituents of compounds of structure (I) or (II) to form embodiments of the invention not specifically set forth above. In addition, in the event that a list of substitutents is listed for any particular substituent group in a particular embodiment and/or claim, it is understood that each individual substituent may be deleted from the particular embodment and/or claim and that the remaining list of substituents will be considered to be within the scope of the invention.

Sisomicin and analogs, stereoisomers, pharmaceutically acceptable salts and prodrugs thereof, including the compounds of structure (A), (I) and (II), may be synthesized according to methods known in the art and described in, for example, International PCT Patent Application Publication No. WO 2009/067692; U.S. Provisional Patent Application Nos. 61/178,834 and 61/312,356; Davies, D. H., et al., J. Med. Chem. 21:189-193 (1978); Nagabhushan, T. L., et al., J. Antibiotics 43-54 (1978); Dena L. Boxler, D. L., et al., JCS Perkin 2168-2185 (1981); Kotretsou, S., et al., J. Med. Chem., 38:4710-4719 (1995); and Hooper, I. R., et al., “Aminoglycoside Antibiotics,” (Springer Verlag, Berlin 1982). In certain embodiments, sisomicin and analogs, stereoisomers, pharmaceutically acceptable salts and prodrugs thereof, including the compounds of structure (A), (I) and (II), are synthesized as described in the Examples herein.

2. Kanamycin and Analogs Thereof

Methods of the present invention may be practiced using kanamycin, or an analog, stereoisomer, pharmaceutically acceptable salt or prodrug thereof, including but not limited to those described herein.

In one embodiment of the present invention, methods are practiced using kanamycin.

In one embodiment of the present invention, methods are practiced using kanamycin analogs, or compounds, having the following structure (B):

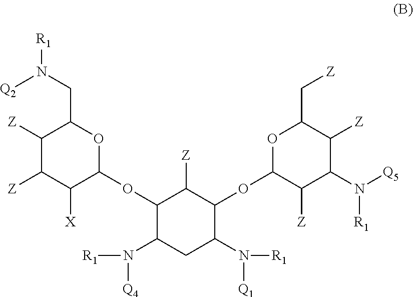

or a stereoisomer, pharmaceutically acceptable salt or prodrug thereof,

wherein:

X is —OR₂ or —NR₁Q₃;

each Z is, independently, halogen or —OR₂;

Q₁, Q₂, Q₃, Q4 and Q₅ are, independently, hydrogen, optionally substituted alkyl, optionally substituted thioalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, or optionally substituted heteroarylalkyl;

each R₁ is, independently, hydrogen or an amino protecting group; and

each R₂ is, independently, hydrogen or a hydroxyl protecting group.

In one embodiment of the present invention, methods are practiced using kanamycin analogs, or compounds, having the following structure (III):

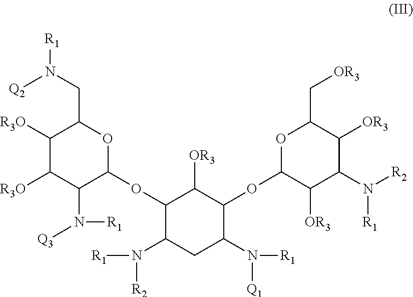

or a stereoisomer, pharmaceutically acceptable salt or prodrug thereof,

wherein:

Q₁ is alkyl optionally substituted with hydroxyl or amino,

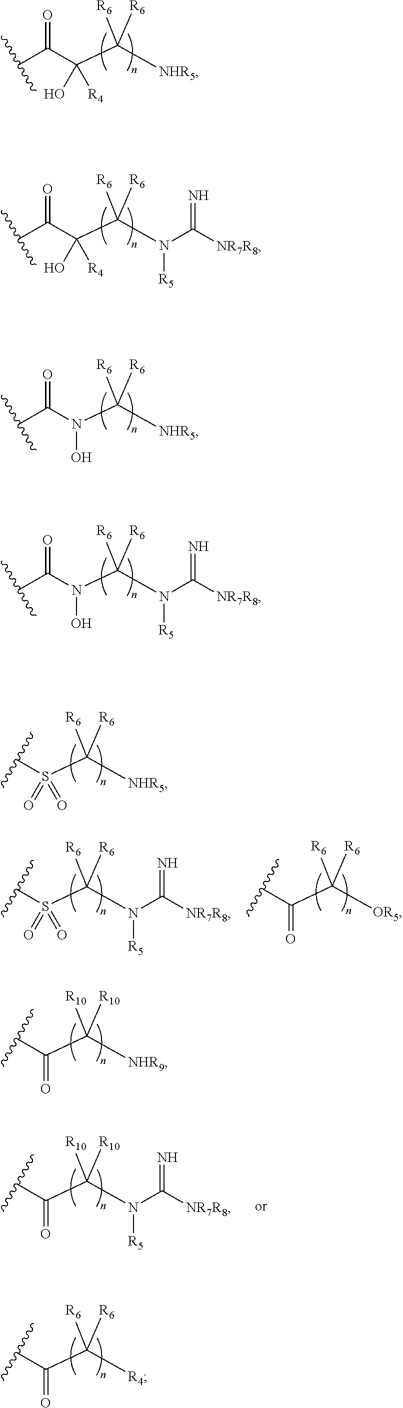

Q₂ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

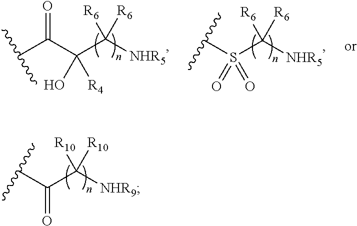

Q₃ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

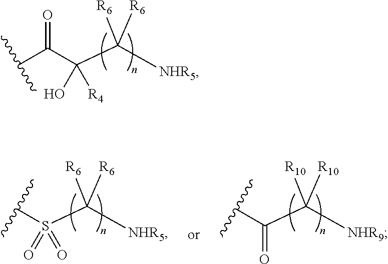

each R₁ and R₂ is, independently, hydrogen or an amino protecting group;

each R₃ is, independently, hydrogen or a hydroxyl protecting group;

each R₄, R₅, R₇ and R₈ is, independently, hydrogen or C₁-C₆ alkyl optionally substituted with one or more halogen, hydroxyl or amino;

each R₆ is, independently, hydrogen, halogen, hydroxyl, amino or C₁-C₆ alkyl;

or R₄ and R₅ together with the atoms to which they are attached can form a heterocyclic ring having from 4 to 6 ring atoms, or R₅ and one R₆ together with the atoms to which they are attached can form a heterocyclic ring having from 3 to 6 ring atoms, or R₄ and one R₆ together with the atoms to which they are attached can form a carbocyclic ring having from 3 to 6 ring atoms, or R₇ and R₈ together with the atom to which they are attached can form a heterocyclic ring having from 3 to 6 ring atoms;

each R₉ is, independently, hydrogen, hydroxyl, amino or C₁-C₆ alkyl optionally substituted with one or more halogen, hydroxyl or amino;

each R₁₀ is, independently, hydrogen, halogen, hydroxyl, amino or C₁-C₆ alkyl;

or R₉ and one R₁₀ together with the atoms to which they are attached can form a heterocyclic ring having from 3 to 6 ring atoms; and

each n is, independently, an integer from 0 to 4, and

wherein (i) at least one of Q₂ and Q₃ are other than hydrogen, and (ii) if Q₁ is —C(═O)OC(CH₃)₃, —C(═O)CH(OH)CH₂NH₂, —C(═O)CH(OH)(CH₂)₂NH₂, or —C(═O)CH(OH)(CH₂)₃NH₂, then Q₂ is not methyl or —C(═O)OC(CH₃)₃, and (iii) if Q₁ is —C(═O)OC(CH₃)₃, —C(═O)CH(OH)CH₂NH₂ or —C(═O)CH(OH)(CH₂)₂NH₂, then Q₃ is not —C(═O)OC(CH₃)₃, —C(═O)OCH₂Ph, —C(═O)CH(OH)CH₂NH₂ or —C(═O)CH(OH)(CH₂)₂NH₂.

In further embodiments:

Q₁ is alkyl optionally substituted with hydroxyl or amino, —C(═O)H,

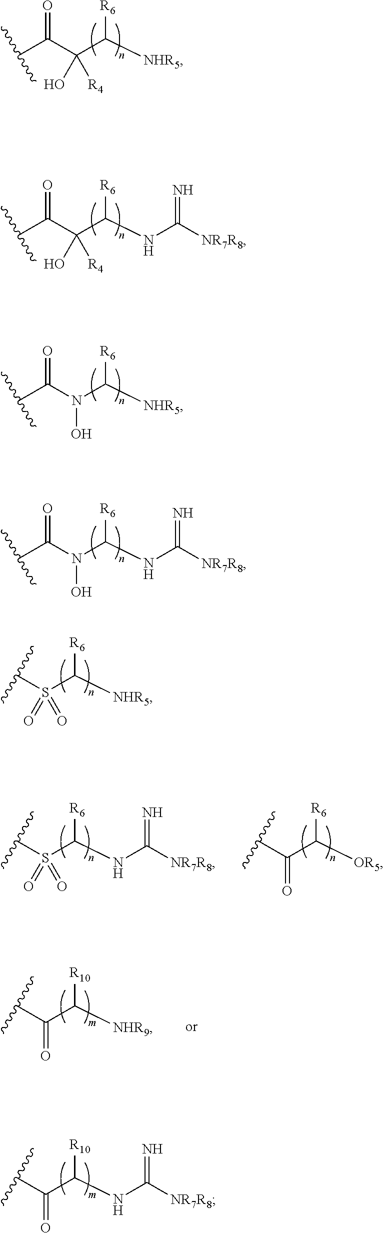

Q₂ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

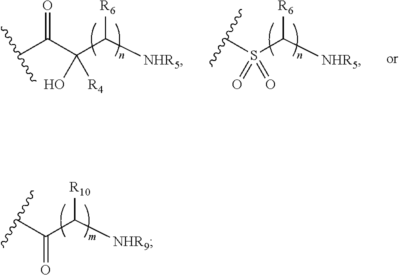

Q₃ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

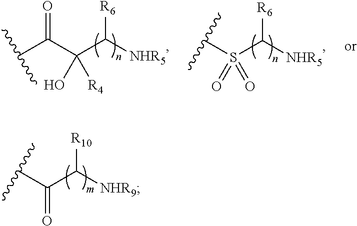

each R₁ and R₂ is, independently, hydrogen or an amino protecting group;

each R₃ is, independently, hydrogen or a hydroxyl protecting group;

each R₄, R₅, R₆, R₇ and R₈ is, independently, hydrogen or C₁-C₆ alkyl, or R₄ and R₅ together with the atoms to which they are attached can form a heterocyclic ring having from 4 to 6 ring atoms, or R₅ and R₆ together with the atoms to which they are attached can form a heterocyclic ring having from 4 to 6 ring atoms, or R₄ and R₆ together with the atoms to which they are attached can form a carbocyclic ring having from 3 to 6 ring atoms, or R₇ and R₈ together with the atom to which they are attached can form a heterocyclic ring having from 4 to 6 ring atoms;

each R₉ and R₁₀ is, independently, hydrogen, hydroxyl, amino or C₁-C₆ alkyl, or R₉ and R₁₀ together with the atoms to which they are attached can form a heterocyclic ring having from 4 to 6 ring atoms;

each n is, independently, an integer from 0 to 4; and

each m is, independently, an integer from 0 to 4, and

wherein (i) at least one of Q₂ and Q₃ are other than hydrogen, and (ii) if Q₁ is —C(═O)OC(CH₃)₃, —C(═O)CH(OH)CH₂NH₂, —C(═O)CH(OH)(CH₂)₂NH₂, or —C(═O)CH(OH)(CH₂)₃NH₂, then Q₂ is not methyl or —C(═O)OC(CH₃)₃, and (iii) if Q₁ is —C(═O)OC(CH₃)₃, —C(═O)CH(OH)CH₂NH₂ or —C(═O)CH(OH)(CH₂)₂NH₂, then Q₃ is not —C(═O)OC(CH₃)₃, —C(═O)OCH₂ Ph, —C(═O)CH(OH)CH₂NH₂ or —C(═O)CH(OH)(CH₂)₂NH₂.

In one embodiment of the present invention, methods are practiced using kanamycin analogs, or compounds, having the following structure (IV):

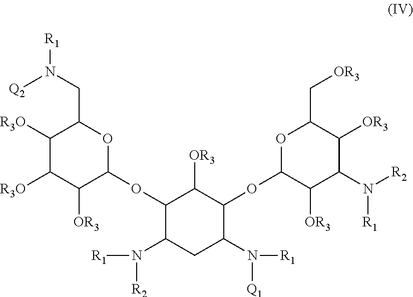

or a stereoisomer, pharmaceutically acceptable salt or prodrug thereof,

wherein:

Q₁ is alkyl optionally substituted with hydroxyl or amino,

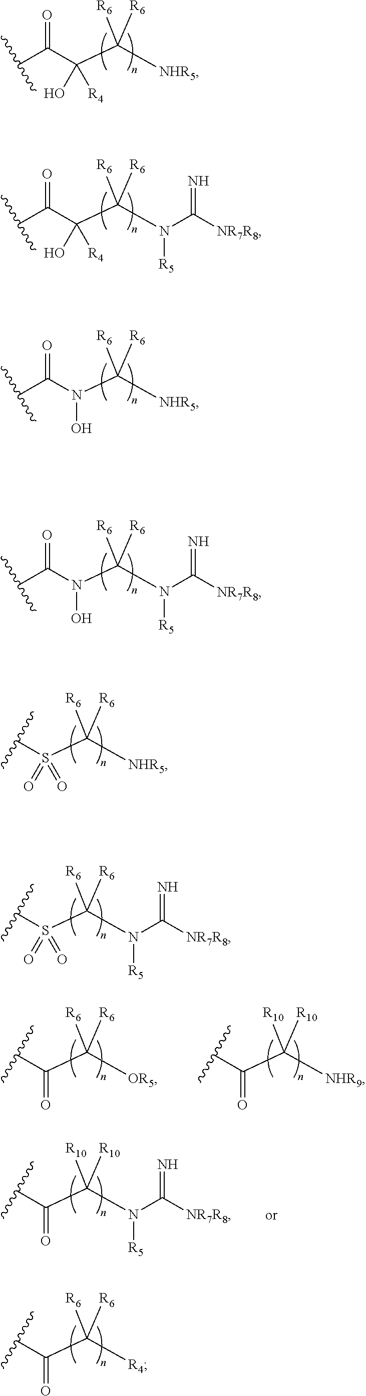

Q₂ is optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

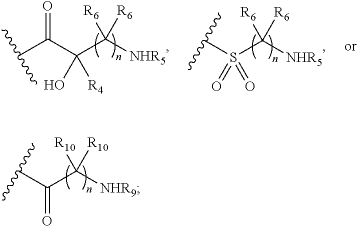

each R₁ and R₂ is, independently, hydrogen or an amino protecting group;

each R₃ is, independently, hydrogen or a hydroxyl protecting group;

each R₄, R₅, R₇ and R₈ is, independently, hydrogen or C₁-C₆ alkyl optionally substituted with one or more halogen, hydroxyl or amino;

each R₆ is, independently, hydrogen, halogen, hydroxyl, amino or C₁-C₆ alkyl;

or R₄ and R₅ together with the atoms to which they are attached can form a heterocyclic ring having from 4 to 6 ring atoms, or R₅ and one R₆ together with the atoms to which they are attached can form a heterocyclic ring having from 3 to 6 ring atoms, or R₄ and one R₆ together with the atoms to which they are attached can form a carbocyclic ring having from 3 to 6 ring atoms, or R₇ and R₈ together with the atom to which they are attached can form a heterocyclic ring having from 3 to 6 ring atoms;

each R₉ is, independently, hydrogen, hydroxyl, amino or C₁-C₆ alkyl optionally substituted with one or more halogen, hydroxyl or amino;

each R₁₀ is, independently, hydrogen, halogen, hydroxyl, amino or C₁-C₆ alkyl;

or R₉ and one R₁₀ together with the atoms to which they are attached can form a heterocyclic ring having from 3 to 6 ring atoms; and

each n is, independently, an integer from 0 to 4, and

wherein (i) if Q₁ is —C(═O)CHOH(CH₂)₂NHRs, then Q₂ is not optionally substituted alkyl, and (ii) if Q₁ is —C(═O)CH(OH)(CH₂)₂NHC(═NH)NH₂, —C(═O)CH(CH₃)CH₂NH₂, —CH₂CH(OH)(CH₂)₂NH₂, —C(═O)CH(OH)CH₂NH₂, —C(═O)CH(OH)(CH₂)₂NH₂, —C(═O)CH(OH)(CH₂)₃NH₂, —C(═O)CHF(CH₂)₂NH₂ or —C(═O)CH₃, then Q₂ is not methyl, ethyl, —CH(═NH), —C(CH₃)(═NH), —C(═NH)NH₂, —C(═O)CH₃, —C(═O)(CH₂)₂CO₂H, —C(═O)CH(OH)(CH₂)₂NH₂,

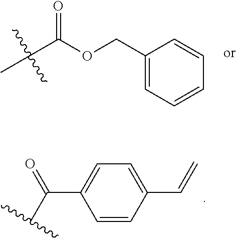

In further embodiments:

Q₁ is alkyl optionally substituted with hydroxyl or amino, —C(═O)H,

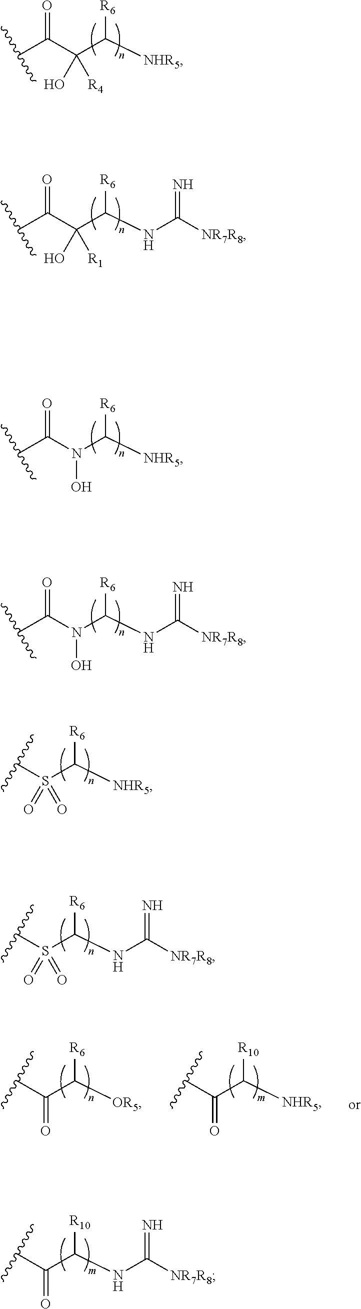

Q₂ is optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

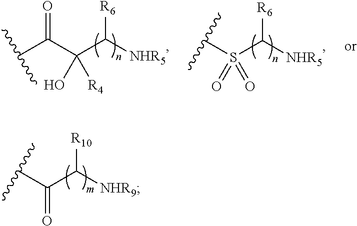

each R₁ and R₂ is, independently, hydrogen or an amino protecting group;

each R₃ is, independently, hydrogen or a hydroxyl protecting group;

each R₄, R₅, R₆, R₇ and R₈ is, independently, hydrogen or C₁-C₆ alkyl, or R₄ and R₅ together with the atoms to which they are attached can form a heterocyclic ring having from 4 to 6 ring atoms, or R₅ and R₆ together with the atoms to which they are attached can form a heterocyclic ring having from 4 to 6 ring atoms, or R₄ and R₆ together with the atoms to which they are attached can form a carbocyclic ring having from 3 to 6 ring atoms, or R₇ and R₈ together with the atom to which they are attached can form a heterocyclic ring having from 4 to 6 ring atoms;

each R₉ and R₁₀ is, independently, hydrogen, hydroxyl, amino or C₁-C₆ alkyl, or R₉ and R₁₀ together with the atoms to which they are attached can form a heterocyclic ring having from 4 to 6 ring atoms;

each n is, independently, an integer from 0 to 4; and

each m is, independently, an integer from 0 to 4, and

wherein if Q₁ is —C(═O)CH(OH)(CH₂)₂NHC(═NH)NH₂, —C(═O)CH(CH₃)CH₂NH₂, —CH₂CH(OH)(CH₂)₂NH₂, —C(═O)CH(OH)CH₂NH₂, —C(═O)CH(OH)(CH₂)₂NH₂, or —C(═O)CH(OH)(CH₂)₃NH₂, then Q₂ is not methyl, ethyl, —CH(═NH), —C(CH₃)(═NH), —C(═NH)NH₂, —C(═O)CH₃, —C(═O)(CH₂)₂CO₂H, —C(═O)CH(OH)(CH₂)₂NH₂,

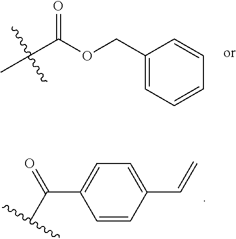

In other further embodiments of structures (III) and (IV), Q₁, Q₂, Q₃ are as set forth above with respect to structures (I) and (II).

It is understood that any embodiment of the compounds of structure (II) or (IV), as set forth above, and any specific substituent set forth herein for a Q₁, Q₂, Q₃, R₁, R₂, R₃, R₄, R₅, R₆, R₇, R₈, R₉ or R₁₀ group in the compounds of structure (III) or (IV), as set forth above, may be independently combined with other embodiments and/or substituents of compounds of structure (III) or (IV) to form embodiments of the invention not specifically set forth above. In addition, in the event that a list of substitutents is listed for any particular substituent group in a particular embodiment and/or claim, it is understood that each individual substituent may be deleted from the particular embodment and/or claim and that the remaining list of substituents will be considered to be within the scope of the invention.

Kanamycin and analogs, stereoisomers, pharmaceutically acceptable salts and prodrugs thereof, including the compounds of structure (B), (III) and (IV), may be synthesized according to methods known in the art and described in, for example, U.S. Provisional Patent Application Nos. 61/178,809 and 61/312,349; Van Schepdael, A., et al., J. Med. Chem. 34:1468-1475 (1991); Van Schepdael, A., et al., J. Med. Chem. 34:1483-1492 (1991); Li, J., et al., Organic Letters 7:3061-3064 (2005); Kotretsou, S., et al., J. Med. Chem. 38:4710-4719 (1995); Jinhua Wang, J., et al., “Aminoglycoside Antibiotics, Chapter 4: Design, Chemical Synthesis, and

Antibacterial Activity of Kanamycin and Neomycin Class Aminoglycoside

Antibiotics,” (John Wiley & Sons, Inc. 2007); and Hooper, I. R., et al., “Aminoglycoside Antibiotics,” (Springer Verlag, Berlin 1982). In certain embodiments, kanamycin and analogs, stereoisomers, pharmaceutically acceptable salts and prodrugs thereof, including the compounds of structure (B), (III) and (IV), are synthesized as described in the Examples herein. As one of skill in the art will appreciate, compounds of structure (IV) wherein Q₁ is 4-amino-2-hydroxy-butyryl may also be referred to as amikacin analogs.

3. Dibekacin and Analogs Thereof

Methods of the present invention may be practiced using dibekacin, or an analog, stereoisomer, pharmaceutically acceptable salt or prodrug thereof, including but not limited to those described herein.

In one embodiment of the present invention, methods are practiced using dibekacin.

In one embodiment of the present invention, methods are practiced using dibekacin analogs, or compounds, having the following structure (C):

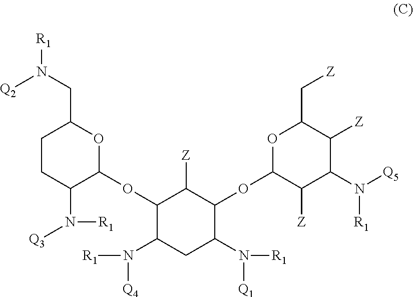

or a stereoisomer, pharmaceutically acceptable salt or prodrug thereof,

wherein:

Q₁, Q₂, Q₃, Q4 and Q₅ are, independently, hydrogen, optionally substituted alkyl, optionally substituted thioalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, or optionally substituted heteroarylalkyl;

each Z is, independently, halogen or —OR₂;

each R₁ is, independently, hydrogen or an amino protecting group; and

each R₂ is, independently, hydrogen or a hydroxyl protecting group.

In one embodiment of the present invention, methods are practiced using dibekacin analogs, or compounds, having the following structure (V):

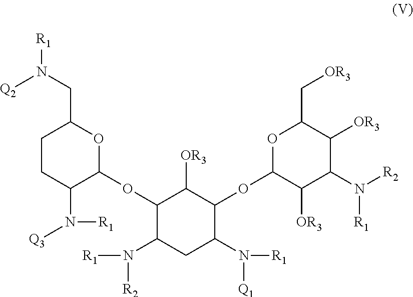

or a stereoisomer, pharmaceutically acceptable salt or prodrug thereof,

wherein:

Q₁ is optionally substituted alkyl,

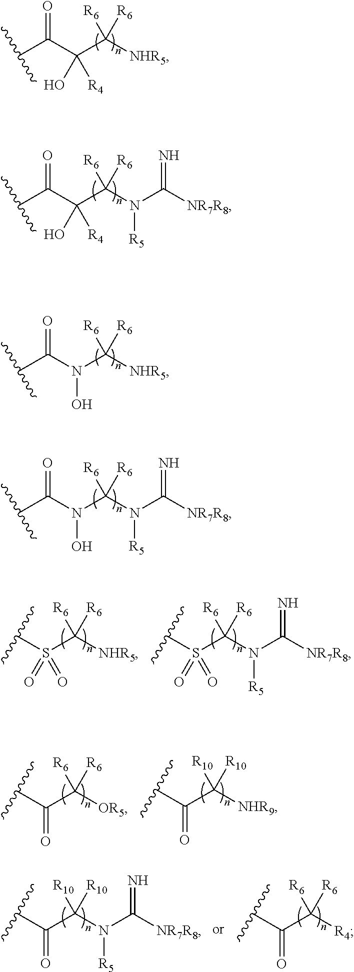

Q₂ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

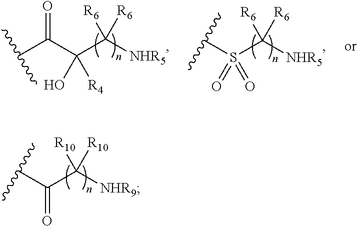

Q₃ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

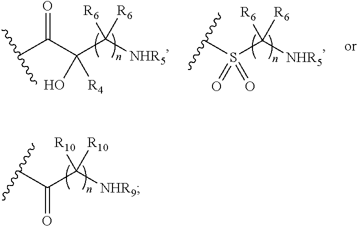

each R₁ and R₂ is, independently, hydrogen or an amino protecting group;

each R₃ is, independently, hydrogen or a hydroxyl protecting group;

each R₄, R₅, R₇ and R₈ is, independently, hydrogen or C₁-C₆ alkyl optionally substituted with one or more halogen, hydroxyl or amino;

each R₆ is, independently, hydrogen, halogen, hydroxyl, amino or C₁-C₆ alkyl;

or R₄ and R₅ together with the atoms to which they are attached can form a heterocyclic ring having from 4 to 6 ring atoms, or R₅ and one R₆ together with the atoms to which they are attached can form a heterocyclic ring having from 3 to 6 ring atoms, or R₄ and one R₆ together with the atoms to which they are attached can form a carbocyclic ring having from 3 to 6 ring atoms, or R₇ and R₈ together with the atom to which they are attached can form a heterocyclic ring having from 3 to 6 ring atoms;

each R₉ is, independently, hydrogen, hydroxyl, amino or C₁-C₆ alkyl optionally substituted with one or more halogen, hydroxyl or amino; each R₁₀ is, independently, hydrogen, halogen, hydroxyl, amino or C₁-C₆ alkyl;

or R₉ and one R₁₀ together with the atoms to which they are attached can form a heterocyclic ring having from 3 to 6 ring atoms; and

n is an integer from 0 to 4, and

wherein (i) at least one of Q₂ and Q₃ are other than hydrogen, and (ii) if Q₁ is —C(═O)CH(OH)CH₂NH₂, —C(═O)CH(OH)(CH₂)₂NH₂, or —C(═O)CH(OH)(CH₂)₃NH₂, then Q₂ is not methyl, —C(═NH)NH₂, —CH(═NH) or —C(═O)CH₃, and (iii) if Q₁ is —C(═O)CH(OH)CH₂NH₂ or —C(═O)CH(OH)(CH₂)₂NH₂, then Q₃ is not —C(═O)CH(OH)CH₂NH₂, —C(═O)CH(OH)(CH₂)₂NH₂ or —C(═O)CH₃.

In further embodiments:

Q₁ is optionally substituted alkyl, —C(═O)H,

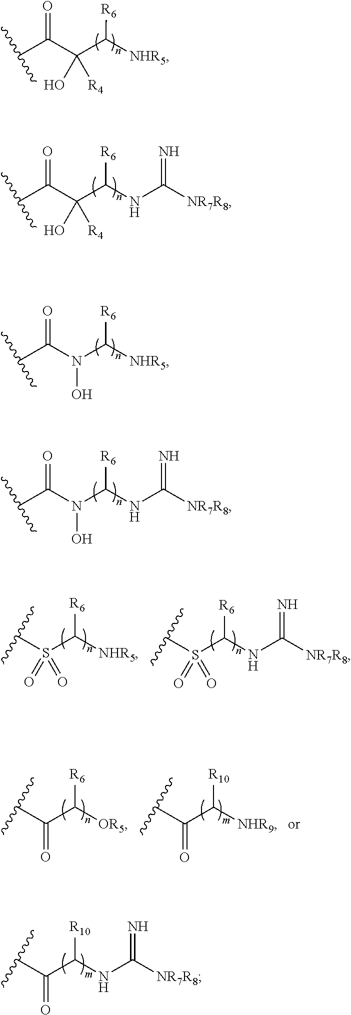

Q₂ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

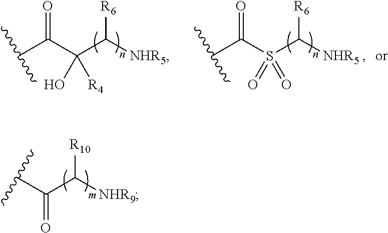

Q₃ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

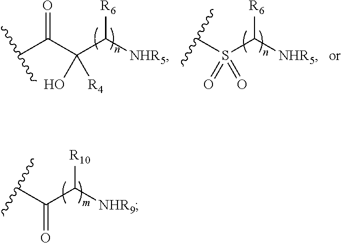

each R₁ and R₂ is, independently, hydrogen or an amino protecting group;

each R₃ is, independently, hydrogen or a hydroxyl protecting group;

each R₄, R₅, R₆, R₇ and R₈ is, independently, hydrogen or C₁-C₆ alkyl, or R₄ and R₅ together with the atoms to which they are attached can form a heterocyclic ring having from 4 to 6 ring atoms, or R₅ and R₆ together with the atoms to which they are attached can form a heterocyclic ring having from 4 to 6 ring atoms, or R₄ and R₆ together with the atoms to which they are attached can form a carbocyclic ring having from 3 to 6 ring atoms, or R₇ and R₈ together with the atom to which they are attached can form a heterocyclic ring having from 4 to 6 ring atoms;

each R₉ and R₁₀ is, independently, hydrogen, hydroxyl, amino or C₁-C₆ alkyl, or R₉ and R₁₀ together with the atoms to which they are attached can form a heterocyclic ring having from 4 to 6 ring atoms;

each n is, independently, an integer from 0 to 4; and

each m is, independently, an integer from 0 to 4, and

wherein (i) at least one of Q₂ and Q₃ are other than hydrogen, and (ii) if Q₁ is —C(═O)CH(OH)CH₂NH₂, —C(═O)CH(OH)(CH₂)₂NH₂, or —C(═O)CH(OH)(CH₂)₃NH₂, then Q₂ is not methyl, —C(═NH)NH₂, —CH(═NH) or —C(═O)CH₃, and (iii) if Q₁ is —C(═O)CH(OH)CH₂NH₂ or —C(═O)CH(OH)(CH₂)₂NH₂, then Q₃ is not —C(═O)CH(OH)CH₂NH₂, —C(═O)CH(OH)(CH₂)₂NH₂ or —C(═O)CH₃.

In other further embodiments of structure (V), Q₁, Q₂, Q₃ are as set forth above with respect to structures (I) and (II).

It is understood that any embodiment of the compounds of structure (V), as set forth above, and any specific substituent set forth herein for a Q₁, Q₂, Q₃, R₁, R₂, R₃, R₄, R₅, R₆, R₇, R₈, R₉ or R₁₀ group in the compounds of structure (V), as set forth above, may be independently combined with other embodiments and/or substituents of compounds of structure (V) to form embodiments of the invention not specifically set forth above. In addition, in the event that a list of substitutents is listed for any particular substituent group in a particular embodiment and/or claim, it is understood that each individual substituent may be deleted from the particular embodment and/or claim and that the remaining list of substituents will be considered to be within the scope of the invention.

Dibekacin and analogs, stereoisomers, pharmaceutically acceptable salts and prodrugs thereof, including the compounds of structure (C) and (V), may be synthesized according to methods known in the art and described in, for example, U.S. Provisional Patent Application Nos. 61/178,814 and 61/312,351; Kondo, S., et al., J. Antibiotics 47:821-832 (1994); Kondo, S., et al., J. Infect Chemotherapy 5:1-9 (1999); Rai, R., et al., J. Carbohydrate Chem. 24:131-143 (2005); and Hooper, I. R., et al., “Aminoglycoside Antibiotics,” (Springer Verlag, Berlin 1982). In certain embodiments, dibekacin and analogs, stereoisomers, pharmaceutically acceptable salts and prodrugs thereof, including the compounds of structure (C) and (V), are synthesized as described in the Examples herein. As one of skill in the art will appreciate, compounds of structure (V) wherein Q₁ is 4-amino-2-hydroxy-butyryl may also be referred to as arbekacin analogs.

4. Tobramycin and Analogs Thereof

Methods of the present invention may be practiced using tobramycin, or an analog, stereoisomer, pharmaceutically acceptable salt or prodrug thereof, including but not limited to those described herein.

In one embodiment of the present invention, methods are practiced using tobramycin.

In one embodiment of the present invention, methods are practiced using tobramycin analogs, or compounds, having the following structure (D):

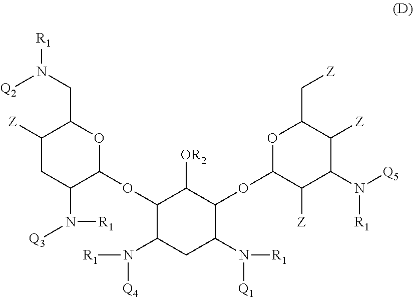

or a stereoisomer, pharmaceutically acceptable salt or prodrug thereof,

wherein:

Q₁, Q₂, Q₃, Q₄ and Q₅ are, independently, hydrogen, optionally substituted alkyl, optionally substituted thioalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, or optionally substituted heteroarylalkyl;

each Z is, independently, halogen or —OR₂;

each R₁ is, independently, hydrogen or an amino protecting group; and

each R₂ is, independently, hydrogen or a hydroxyl protecting group.

In one embodiment of the present invention, methods are practiced using tobramycin analogs, or compounds, having the following structure (VI):

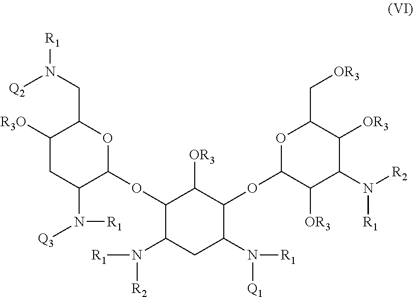

or a stereoisomer, pharmaceutically acceptable salt or prodrug thereof,

wherein:

Q₁ is optionally substituted alkyl,

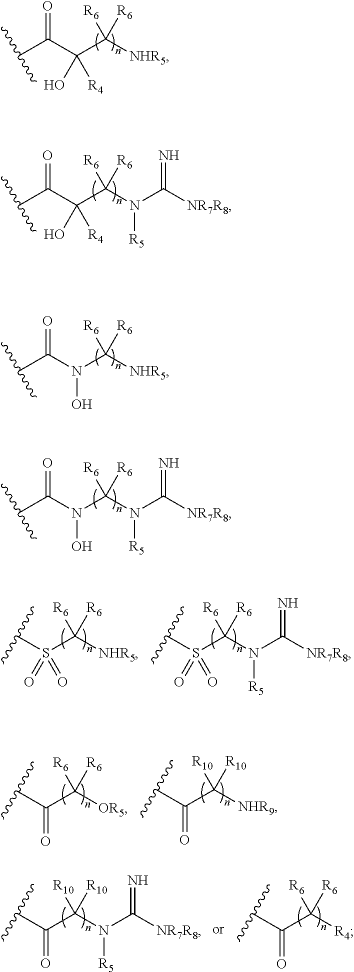

Q₂ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

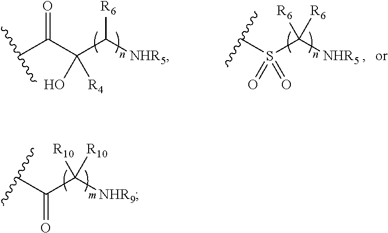

Q₃ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

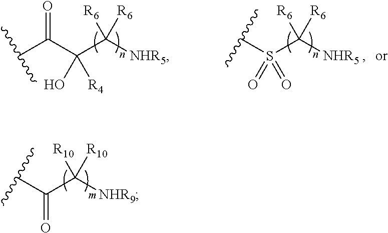

each R₁ and R₂ is, independently, hydrogen or an amino protecting group;

each R₃ is, independently, hydrogen or a hydroxyl protecting group;

each R₄, R₅, R₇ and R₈ is, independently, hydrogen or C₁-C₆ alkyl optionally substituted with one or more halogen, hydroxyl or amino;

each R₆ is, independently, hydrogen, halogen, hydroxyl, amino or C₁-C₆ alkyl;

or R₄ and R₅ together with the atoms to which they are attached can form a heterocyclic ring having from 4 to 6 ring atoms, or R₅ and one R₆ together with the atoms to which they are attached can form a heterocyclic ring having from 3 to 6 ring atoms, or R₄ and one R₆ together with the atoms to which they are attached can form a carbocyclic ring having from 3 to 6 ring atoms, or R₇ and R₈ together with the atom to which they are attached can form a heterocyclic ring having from 3 to 6 ring atoms;

each R₉ is, independently, hydrogen, hydroxyl, amino or C₁-C₆ alkyl optionally substituted with one or more halogen, hydroxyl or amino;

each R₁₀ is, independently, hydrogen, halogen, hydroxyl, amino or C₁-C₆ alkyl;

or R₉ and one R₁₀ together with the atoms to which they are attached can form a heterocyclic ring having from 3 to 6 ring atoms; and

n is an integer from 0 to 4, and

wherein (i) at least one of Q₂ and Q₃ are other than hydrogen, (ii) if Q₁ is —C(═O)CH(OH)(CH₂)₂NH₂, then Q₂ is not methyl, and (iii) Q₁, Q₂ and Q₃ are not all —C(═O)CH₃.

In further embodiments,

Q₁ is optionally substituted alkyl, —C(═O)H,

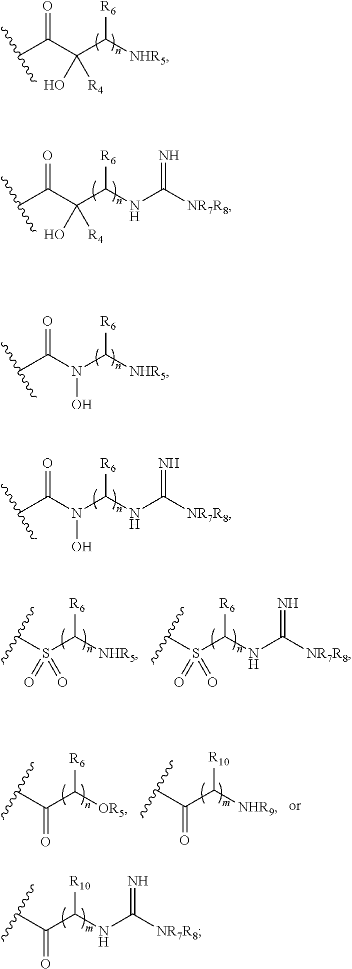

Q₂ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

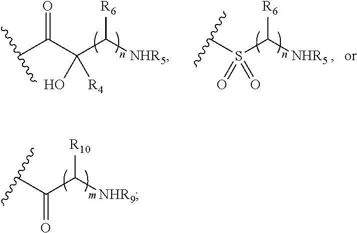

Q₃ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

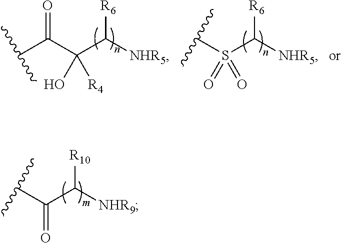

each R₁ and R₂ is, independently, hydrogen or an amino protecting group;

each R₃ is, independently, hydrogen or a hydroxyl protecting group;

each R₄, R₅, R₆, R₇ and R₈ is, independently, hydrogen or C₁-C₆ alkyl, or R₄ and R₅ together with the atoms to which they are attached can form a heterocyclic ring having from 4 to 6 ring atoms, or R₅ and R₆ together with the atoms to which they are attached can form a heterocyclic ring having from 4 to 6 ring atoms, or R₄ and R₆ together with the atoms to which they are attached can form a carbocyclic ring having from 3 to 6 ring atoms, or R₇ and R₈ together with the atom to which they are attached can form a heterocyclic ring having from 4 to 6 ring atoms;

each R₉ and R₁₀ is, independently, hydrogen, hydroxyl, amino or C₁-C₆ alkyl, or R₉ and R₁₀ together with the atoms to which they are attached can form a heterocyclic ring having from 4 to 6 ring atoms;

each n is, independently, an integer from 0 to 4; and

each m is, independently, an integer from 0 to 4, and

wherein (i) at least one of Q₂ and Q₃ are other than hydrogen, (ii) if Q₁ is —C(═O)CH(OH)(CH₂)₂NH₂, then Q₂ is not methyl, and (iii) Q₁, Q₂ and Q₃ are not all —C(═O)CH₃.

In other further embodiments of structure (VI), Q₁, Q₂, Q₃ are as set forth above with respect to structures (I) and (II).

It is understood that any embodiment of the compounds of structure (VI), as set forth above, and any specific substituent set forth herein for a Q₁, Q₂, Q₃, R₁, R₂, R₃, R₄, R₅, R₆, R₇, R₈, R₉ or R₁₀ group in the compounds of structure (VI), as set forth above, may be independently combined with other embodiments and/or substituents of compounds of structure (VI) to form embodiments of the invention not specifically set forth above. In addition, in the event that a list of substitutents is listed for any particular substituent group in a particular embodiment and/or claim, it is understood that each individual substituent may be deleted from the particular embodment and/or claim and that the remaining list of substituents will be considered to be within the scope of the invention.

Tobramycin and analogs, stereoisomers, pharmaceutically acceptable salts and prodrugs thereof, including the compounds of structure (D) and (VI) may be synthesized according to methods known in the art and described in, for example, U.S. Provisional Patent Application Nos. 61/178,826 and 61/312,353; Jinhua Wang, J., et al., “Aminoglycoside Antibiotics, Chapter 4: Design, Chemical Synthesis, and Antibacterial Activity of Kanamycin and Neomycin Class Aminoglycoside Antibiotics,” (John Wiley & Sons, Inc. 2007); Hooper, I. R., et al., “Aminoglycoside Antibiotics,” (Springer Verlag, Berlin 1982); Tanabe, M., et al., Tetrahedron Letters 41:3607-3610 (1977); and European Patent No. 0009670. In certain embodiments, tobramycin and analogs, stereoisomers, pharmaceutically acceptable salts and prodrugs thereof, including the compounds of structure (D) and (VI), are synthesized as further described in the Examples herein.

5. Gentamicin and Analogs Thereof

Methods of the present invention may be practiced using gentamicin, or an analog, stereoisomer, pharmaceutically acceptable salt or prodrug thereof, including but not limited to those described herein.

In one embodiment of the present invention, methods are practiced using gentamicin.

In one embodiment of the present invention, methods are practiced using gentamicin analogs, or compounds, having the following structure (E):

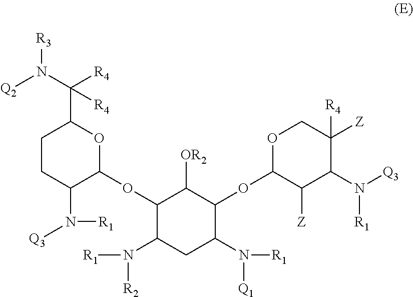

or a stereoisomer, pharmaceutically acceptable salt or prodrug thereof,

wherein:

Q₁, Q₂, Q₃, Q₄ and Q₅ are, independently, hydrogen, optionally substituted alkyl, optionally substituted thioalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, or optionally substituted heteroarylalkyl;

each Z is, independently, halogen or —OR₂;

each R₁ is, independently, hydrogen or an amino protecting group;

each R₂ is, independently, hydrogen or a hydroxyl protecting group;

R₃ is hydrogen, methyl or an amino protecting group; and

each R₄ is, independently, hydrogen or methyl.

In one embodiment of the present invention, methods are practiced using gentamicin analogs, or compounds, having the following structure (VII):

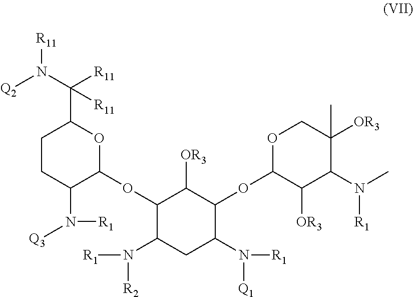

or a stereoisomer, pharmaceutically acceptable salt or prodrug thereof,

wherein:

Q₁ is alkyl optionally substituted with hydroxyl or amino,

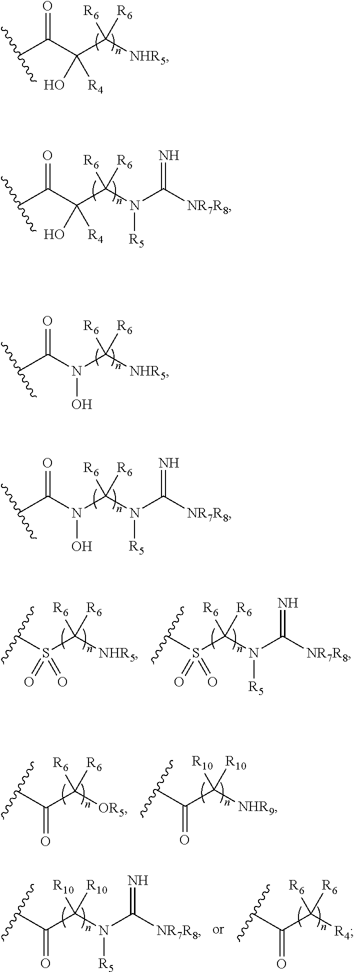

Q₂ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

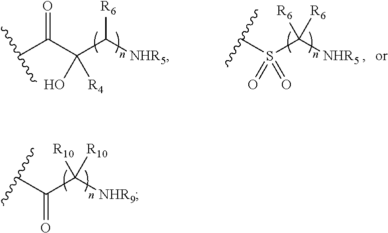

Q₃ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

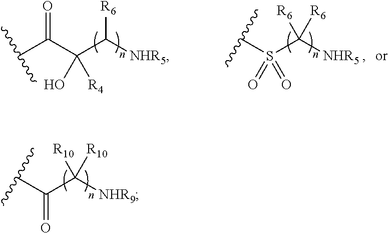

each R₁ and R₂ is, independently, hydrogen or an amino protecting group;

each R₃ is, independently, hydrogen or a hydroxyl protecting group;

each R₄, R₅, R₇ and R₈ is, independently, hydrogen or C₁-C₆ alkyl optionally substituted with one or more halogen, hydroxyl or amino;

each R₆ is, independently, hydrogen, halogen, hydroxyl, amino or C₁-C₆ alkyl;

or R₄ and R₅ together with the atoms to which they are attached can form a heterocyclic ring having from 4 to 6 ring atoms, or R₅ and one R₆ together with the atoms to which they are attached can form a heterocyclic ring having from 3 to 6 ring atoms, or R₄ and one R₆ together with the atoms to which they are attached can form a carbocyclic ring having from 3 to 6 ring atoms, or R₇ and R₈ together with the atom to which they are attached can form a heterocyclic ring having from 3 to 6 ring atoms;

each R₉ is, independently, hydrogen, hydroxyl, amino or C₁-C₆ alkyl optionally substituted with one or more halogen, hydroxyl or amino;

each R₁₀ is, independently, hydrogen, halogen, hydroxyl, amino or C₁-C₆ alkyl;

or R₉ and one R₁₀ together with the atoms to which they are attached can form a heterocyclic ring having from 3 to 6 ring atoms;

each R₁₁ is, independently, hydrogen or methyl; and

each n is, independently, an integer from 0 to 4, and

wherein (i) at least one of Q₂ and Q₃ are other than hydrogen, and (ii) if Q₁ is methyl, ethyl, i-propyl, i-butyl, —(CH₂)₂OH, —CH₂CH(OH)(CH₂)₂NH₂, —C(═O)H, —C(═O)CH(OH)CH₂NH₂, —C(═O)CH(OH)(CH₂)₂NH₂, —C(═O)CH(OH)(CH₂)₃NH₂, —C(═O)CH(OH)(CH₂)₄NH₂, —C(═O)N(OH)(CH₂)₂NH₂, —C(═O)CH₂NH₂, —C(═O)(CH₂)₄NH₂, —C(═O)CH₂NHC(CH₃)₃, —C(═O)CH(CH(CH₂)₂)NH₂, —C(═O)CHNH₂ (CH₂)₄NH₂, —C(═O)(CH₂)₂C(CH₃)₂CH(CH₃)OH, —C(═O)CH(OH)CH₂NH₂, —C(═O)OC(CH₃)₃, —C(═O)(CH₂)₄OH, —C(═O)CH₃ or —C(═O)NH(CH₂)₂NH₂, then Q₂ is not methyl, and (iii) if Q₁ is —C(═O)CH(OH)(CH₂)₂NH₂, then Q₃ is not —C(═O)CH(OH)(CH₂)₂NH₂.

In further embodiments:

Q₁ is alkyl optionally substituted with hydroxyl or amino, —C(═O)H,

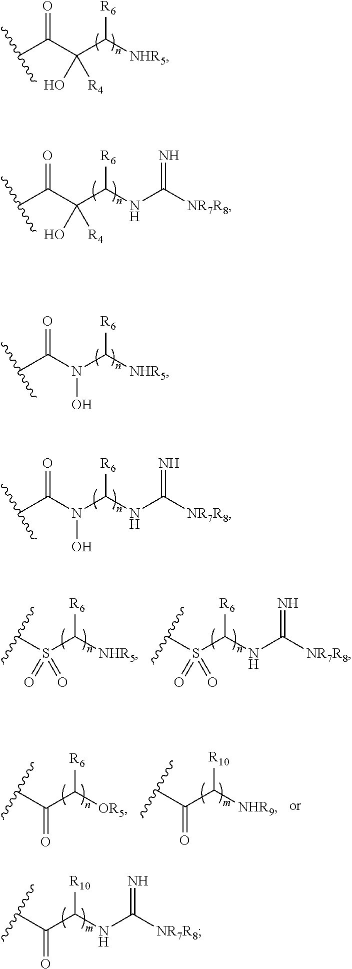

Q₂ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

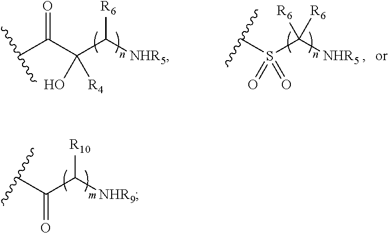

Q₃ is hydrogen, optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

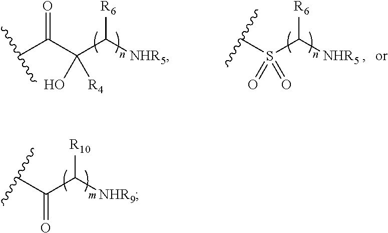

each R₁ and R₂ is, independently, hydrogen or an amino protecting group;

each R₃ is, independently, hydrogen or a hydroxyl protecting group;

each R₄, R₅, R₆, R₇ and R₈ is, independently, hydrogen or C₁-C₆ alkyl, or R₄ and R₅ together with the atoms to which they are attached can form a heterocyclic ring having from 4 to 6 ring atoms, or R₅ and R₆ together with the atoms to which they are attached can form a heterocyclic ring having from 4 to 6 ring atoms, or R₄ and R₆ together with the atoms to which they are attached can form a carbocyclic ring having from 3 to 6 ring atoms, or R₇ and R₈ together with the atom to which they are attached can form a heterocyclic ring having from 4 to 6 ring atoms;

each R₉ and R₁₀ is, independently, hydrogen, hydroxyl, amino or C₁-C₆ alkyl, or R₉ and R₁₀ together with the atoms to which they are attached can form a heterocyclic ring having from 4 to 6 ring atoms;

each R₁₁ is, independently, hydrogen or methyl;

each n is, independently, an integer from 0 to 4; and

each m is, independently, an integer from 0 to 4, and

wherein (i) at least one of Q₂ and Q₃ are other than hydrogen, and (ii) if Q₁ is methyl, ethyl, i-propyl, i-butyl, —(CH₂)₂OH, —CH₂CH(OH)(CH₂)₂NH₂, —C(═O)H, —C(═O)CH(OH)CH₂NH₂, —C(═O)CH(OH)(CH₂)₂NH₂, —C(═O)CH(OH)(CH₂)₃NH₂, —C(═O)CH(OH)(CH₂)₄NH₂, —C(═O)N(OH)(CH₂)₂NH₂, —C(═O)CH₂NH₂, —C(═O)(CH₂)₄NH₂, —C(═O)CH₂NHC(CH₃)₃, —C(═O)CH(CH(CH₂)₂)NH₂, —C(═O)CHNH₂ (CH₂)₄NH₂, —C(═O)(CH₂)₂C(CH₃)₂CH(CH₃)OH, —C(═O)CH(OH)CH₂NH₂, —C(═O)OC(CH₃)₃ or —C(═O)(CH₂)₄OH, then Q₂ is not methyl, and (iii) if Q₁ is —C(═O)CH(OH)(CH₂)₂NH₂, then Q₃ is not —C(═O)CH(OH)(CH₂)₂NH₂.

In one embodiment of the present invention, methods are practiced using gentamicin analogs, or compounds, having the following structure (VIII):

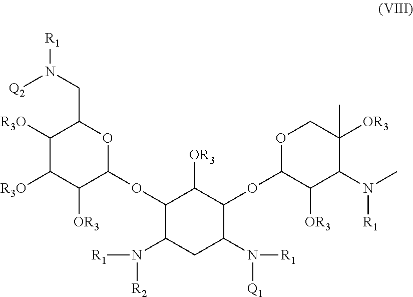

or a stereoisomer, pharmaceutically acceptable salt or prodrug thereof,

wherein:

Q₁ is optionally substituted alkyl,

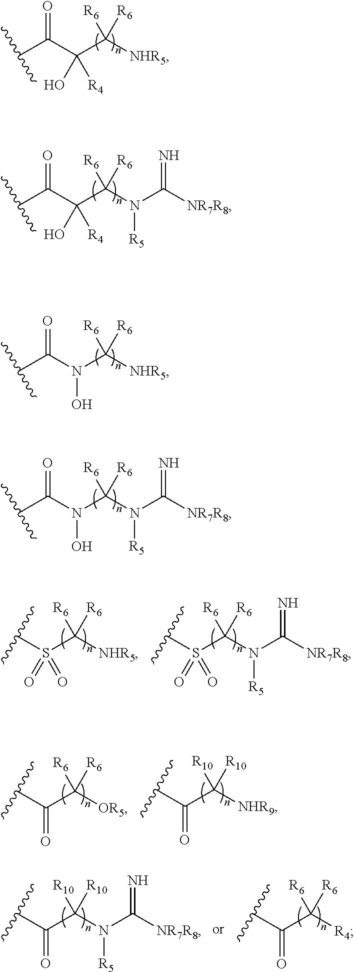

Q₂ is optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

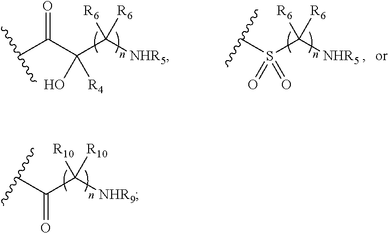

each R₁ and R₂ is, independently, hydrogen or an amino protecting group;

each R₃ is, independently, hydrogen or a hydroxyl protecting group;

each R₄, R₅, R₇ and R₈ is, independently, hydrogen or C₁-C₆ alkyl optionally substituted with one or more halogen, hydroxyl or amino;

each R₆ is, independently, hydrogen, halogen, hydroxyl, amino or C₁-C₆ alkyl;

or R₄ and R₅ together with the atoms to which they are attached can form a heterocyclic ring having from 4 to 6 ring atoms, or R₅ and one R₆ together with the atoms to which they are attached can form a heterocyclic ring having from 3 to 6 ring atoms, or R₄ and one R₆ together with the atoms to which they are attached can form a carbocyclic ring having from 3 to 6 ring atoms, or R₇ and R₈ together with the atom to which they are attached can form a heterocyclic ring having from 3 to 6 ring atoms;

each R₉ is, independently, hydrogen, hydroxyl, amino or C₁-C₆ alkyl optionally substituted with one or more halogen, hydroxyl or amino;

each R₁₀ is, independently, hydrogen, halogen, hydroxyl, amino or C₁-C₆ alkyl;

or R₉ and one R₁₀ together with the atoms to which they are attached can form a heterocyclic ring having from 3 to 6 ring atoms; and

n is an integer from 0 to 4, and

wherein if Q₁ is —C(═O)CH₃, —C(═O)CH(OH)CH₂NH₂, —C(═O)CH(OH)CH₂NH₂ or —C(═O)CH(OH)(CH₂)₂NH₂, then Q₂ is not methyl, —C(═O)CH(OH)CH₂NH₂, —C(═O)OC(CH₃)₃ or

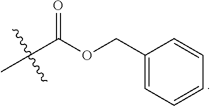

In further embodiments:

Q₁ is optionally substituted alkyl, —C(═O)H,

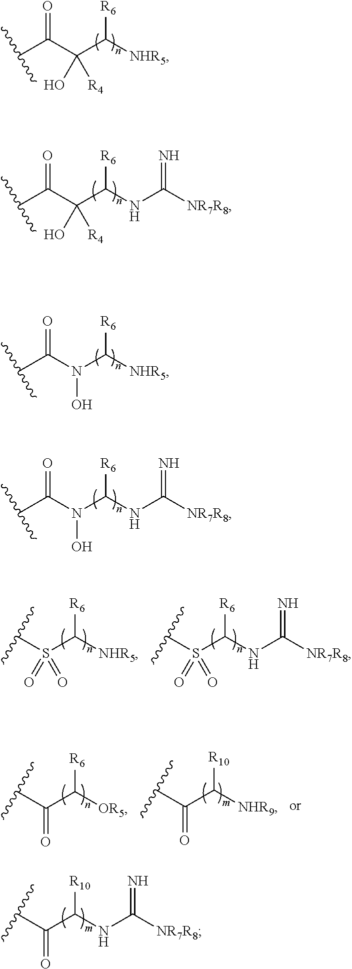

Q₂ is optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₇R₈,

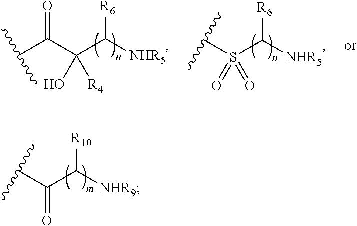

each R₁ and R₂ is, independently, hydrogen or an amino protecting group;

each R₃ is, independently, hydrogen or a hydroxyl protecting group;

each R₄, R₅, R₆, R₇ and R₈ is, independently, hydrogen or C₁-C₆ alkyl, or R₄ and R₅ together with the atoms to which they are attached can form a heterocyclic ring having from 4 to 6 ring atoms, or R₅ and R₆ together with the atoms to which they are attached can form a heterocyclic ring having from 4 to 6 ring atoms, or R₄ and R₆ together with the atoms to which they are attached can form a carbocyclic ring having from 3 to 6 ring atoms, or R₇ and R₈ together with the atom to which they are attached can form a heterocyclic ring having from 4 to 6 ring atoms;

each R₉ and R₁₀ is, independently, hydrogen, hydroxyl, amino or C₁-C₆ alkyl, or R₉ and R₁₀ together with the atoms to which they are attached can form a heterocyclic ring having from 4 to 6 ring atoms;

each n is, independently, an integer from 0 to 4; and

each m is, independently, an integer from 0 to 4, and

wherein if Q₁ is —C(═O)CH₃, —C(═O)CH(OH)CH₂NH₂, —C(═O)CH(OH)CH₂NH₂ or —C(═O)CH(OH)(CH₂)₂NH₂, then Q₂ is not methyl, —C(═O)CH(OH)CH₂NH₂, —C(═O)OC(CH₃)₃ or

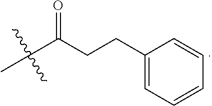

In other further embodiments of structures (VII) and (VIII), Q₁, Q₂, Q₃ are as set forth above with respect to structures (I) and (II).

It is understood that any embodiment of the compounds of structure (VII) or (VIII), as set forth above, and any specific substituent set forth herein for a Q₁, Q₂, Q₃, R₁, R₂, R₃, R₄, R₅, R₆, R₇, R₈, R₉, R₁₀ or R₁₁ group in the compounds of structure (VII) or (VIII), as set forth above, may be independently combined with other embodiments and/or substituents of compounds of structure (VII) or (VIII) to form embodiments of the invention not specifically set forth above. In addition, in the event that a list of substitutents is listed for any particular substituent group in a particular embodiment and/or claim, it is understood that each individual substituent may be deleted from the particular embodment and/or claim and that the remaining list of substituents will be considered to be within the scope of the invention.

Gentamicin and analogs, stereoisomers, pharmaceutically acceptable salts and prodrugs thereof, including the compounds of structure (E), (VII) and (VIII), may be synthesized according to methods known in the art and described in, for example, U.S. Provisional Patent Application Nos. 61/178,854 and 61/312,354;

Nagabhushan, T. L., et al., J. Antibiotics 7:681-687 (1978); Daniels, P. L., et al., Japanese Journal of Antibiotics S-195-S-204 (1979); and Hooper, I. R., et al., “Aminoglycoside Antibiotics,” (Springer Verlag, Berlin 1982). In certain embodiments, gentamicin and analogs, stereoisomers, pharmaceutically acceptable salts and prodrugs thereof, including the compounds of structure (E), (VII) and (VIII), are synthesized as further described in the Examples herein.

6. Neomycin and Analogs Thereof

Methods of the present invention may be practiced using neomycin, or an analog, stereoisomer, pharmaceutically acceptable salt or prodrug thereof, including but not limited to those described herein.

In one embodiment of the present invention, methods are practiced using neomycin.

In one embodiment of the present invention, methods are practiced using neomycin analogs, or compounds, having the following structure (F):

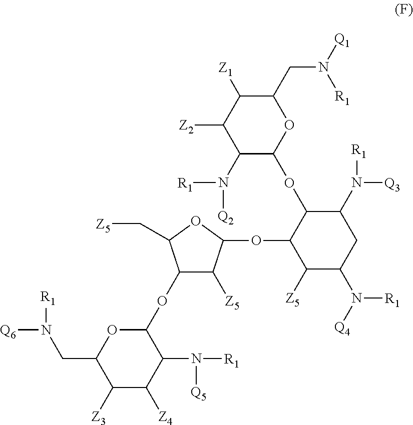

or a stereoisomer, pharmaceutically acceptable salt or prodrug thereof,

wherein:

Q₁, Q₂, Q₃, Q4, Q₅ and Q6 are, independently, hydrogen, optionally substituted alkyl, optionally substituted thioalkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, or optionally substituted heteroarylalkyl;

each R₁ is, independently, hydrogen or an amino protecting group;

each R₂ is, independently, hydrogen or a hydroxyl protecting group;

Z₁ and Z₂ are, independently, hydrogen, halogen or —OR₂, or Z₁ and Z₂ form a double bond;

Z₃ and Z₄ are, independently, hydrogen, halogen or —OR₂, or Z₃ and Z₄ form a double bond; and

each Z₅ is, independently, halogen or —OR₂.

Neomycin and analogs, stereoisomers, pharmaceutically acceptable salts and prodrugs thereof, including the compounds of structure (F), may be synthesized according to methods known in the art and described in, for example, Jinhua Wang, J., et al., “Aminoglycoside Antibiotics, Chapter 4: Design, Chemical Synthesis, and Antibacterial Activity of Kanamycin and Neomycin Class Aminoglycoside Antibiotics,” (John Wiley & Sons, Inc. 2007); Li, J., et al., Organic Letters 7:3061-3064 (2005); and Hooper, I. R., et al., “Aminoglycoside Antibiotics,” (Springer Verlag, Berlin 1982).

### D. Pharmaceutical Compositions and Administation

For the purposes of administration, the aminoglycosides used in the methods of the present invention may be administered as a raw chemical or may be formulated as pharmaceutical compositions. Pharmaceutical compositions of the present invention comprise an aminoglycoside and a pharmaceutically acceptable carrier, diluent or excipient. The antibacterial activity of aminoglycosides and compounds of structure (I), (II), (III), (IV), (V), (VI), (VII) and (VIII) for various bacteria can be determined by one skilled in the art, for example, as described in the Examples below. Appropriate concentrations and dosages can be readily determined by one skilled in the art.

Administration of aminoglycosides, in pure form or in an appropriate pharmaceutical composition, can be carried out via any of the accepted modes of administration of agents for serving similar utilities. The pharmaceutical compositions of the invention can be prepared by combining a compound of the invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000).

The pharmaceutical composition may be in the form of a liquid, for example, an elixir, syrup, solution, emulsion or suspension. The liquid may be for oral administration or for delivery by injection, as two examples. In a composition intended to be administered by injection, one or more of a surfactant, preservative, wetting agent, dispersing agent, suspending agent, buffer, stabilizer and isotonic agent may be included. The parenteral preparation can be enclosed in ampoules, disposable syringes or multiple dose vials made of glass or plastic. Physiological saline is a preferred adjuvant. An injectable pharmaceutical composition is preferably sterile.

As described in the accompanying Examples, aminoglycosides may be administered intravenously. Accordingly, the present invention includes pharmaceutical formulations of aminoglycosides, including the compound, 6′-(2-Hydroxy-ethyl)-1-(4-amino-2(S)-hydroxy-butyryl)-sisomicin, suitable for intravenous infusion, wherein the formulations contain the aminoglycoside at a concentration sufficient to provide at least 10 mg/kg subject body weight, at least 15 mg/kg subject body weight, at least 20 mg/kg subject body weight, at least 25 mg/kg subject body weight, or at least 30 mg/kg subject body weight of the aminoglycoside, e.g., 6′-(2-Hydroxy-ethyl)-1-(4-amino-2(S)-hydroxy-butyryl)-sisomicin, to the subject over an infusion time period of between 10-15 minutes or about 10 minutes, at an acceptable rate of infusion.

The aminoglycosides, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

Aminoglycosides, or pharmaceutically acceptable derivatives thereof, may also be administered simultaneously with, prior to, or after administration of one or more other therapeutic agents. Such combination therapy includes administration of a single pharmaceutical dosage formulation which contains an aminoglycoside and one or more additional active agents, as well as administration of the aminoglycoside and each active agent in its own separate pharmaceutical dosage formulation.

### EXAMPLES

### General Synthetic Schemes

### Sisomicin Analogs

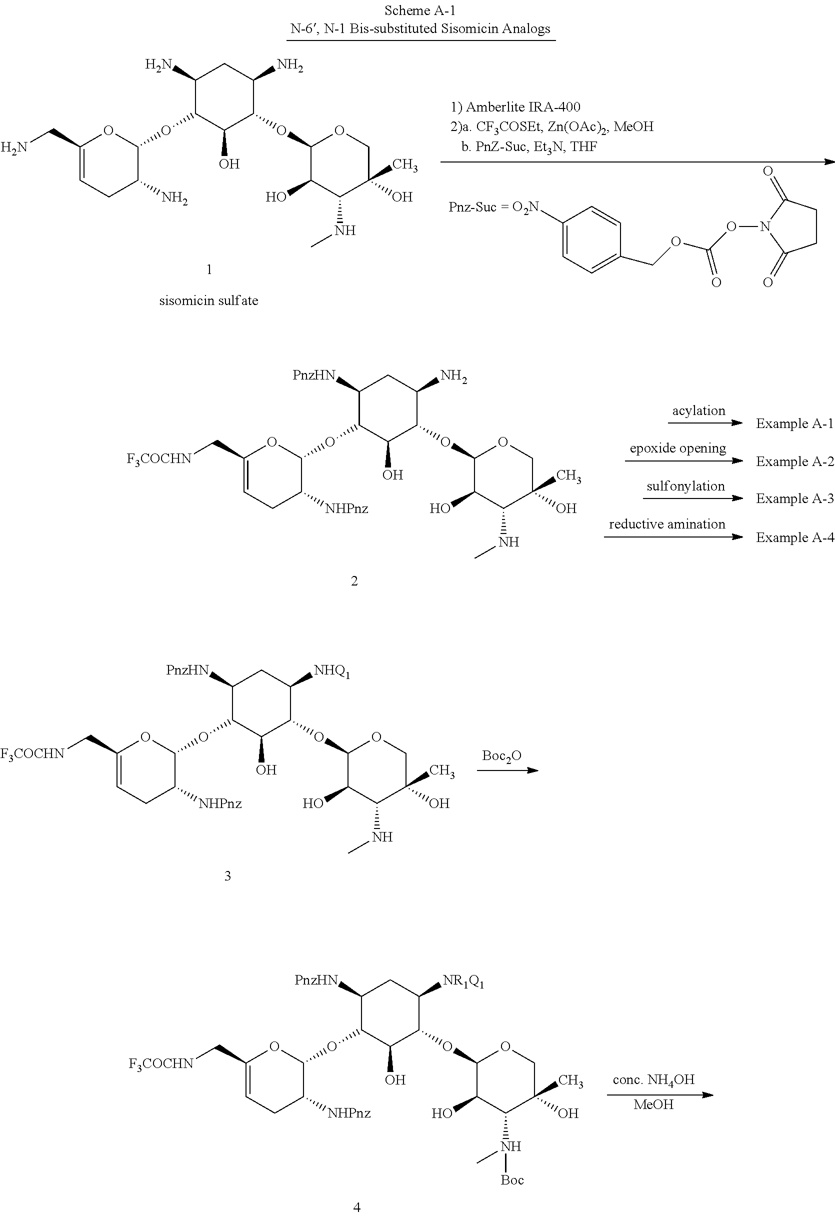

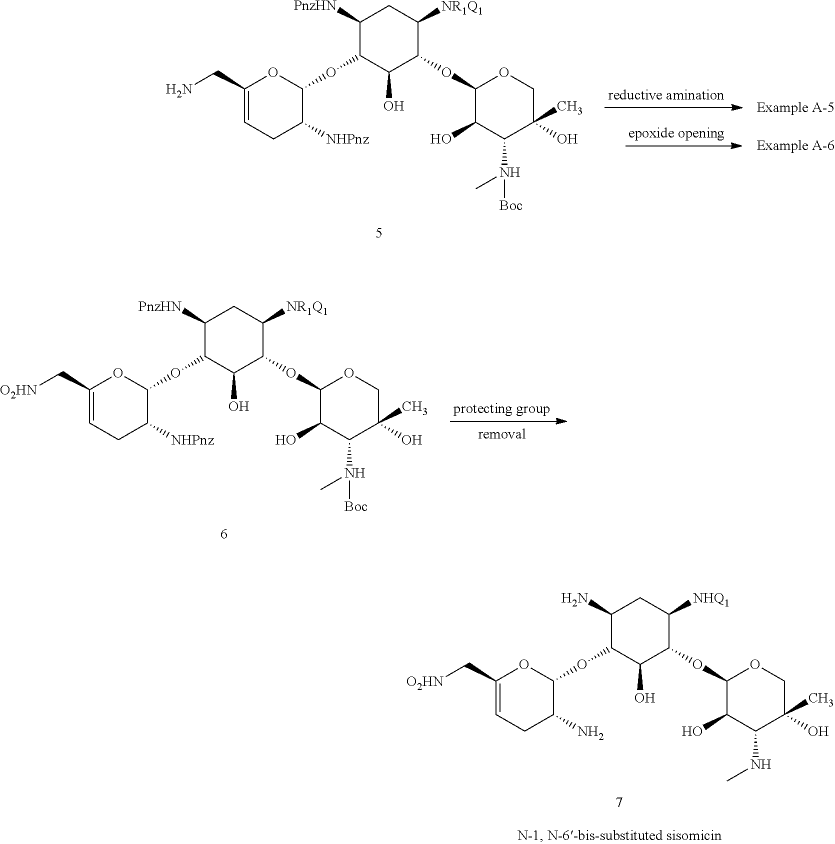

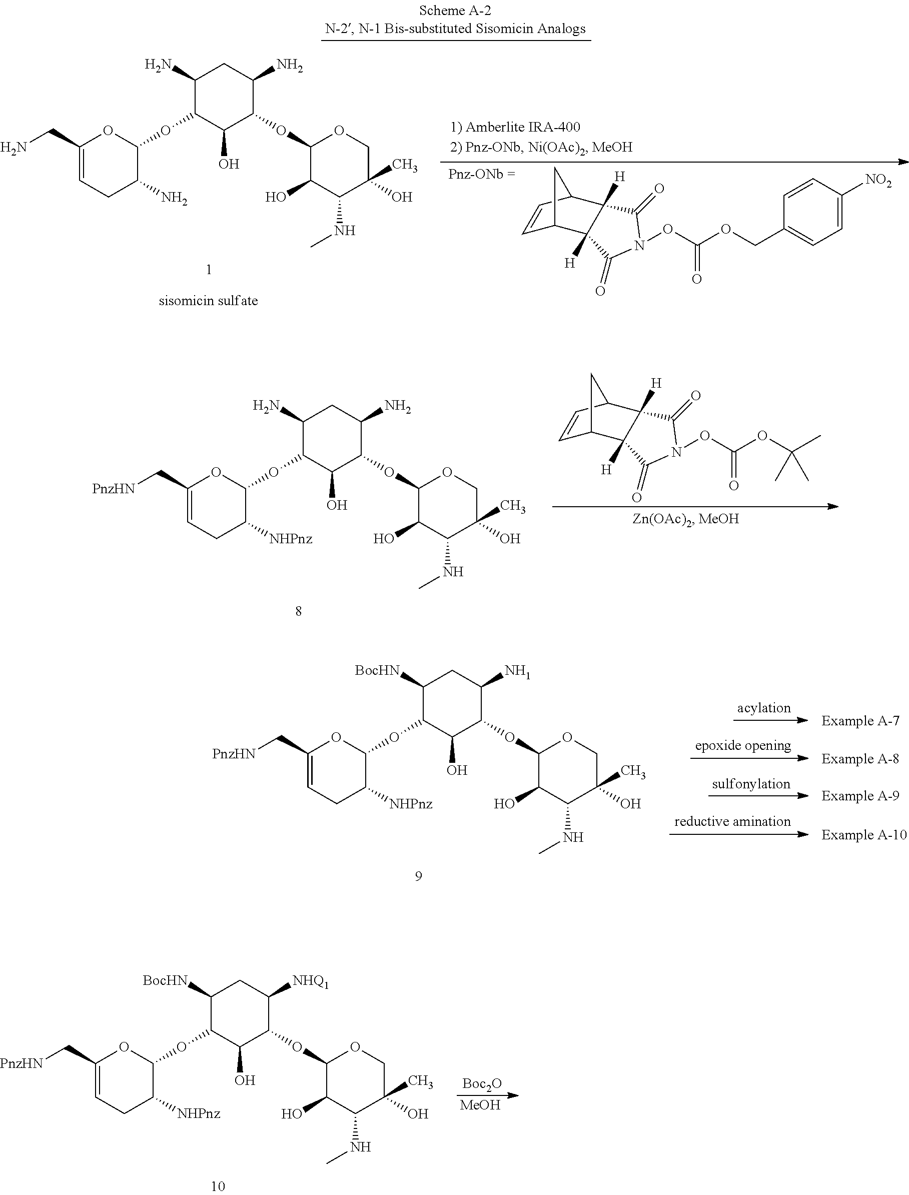

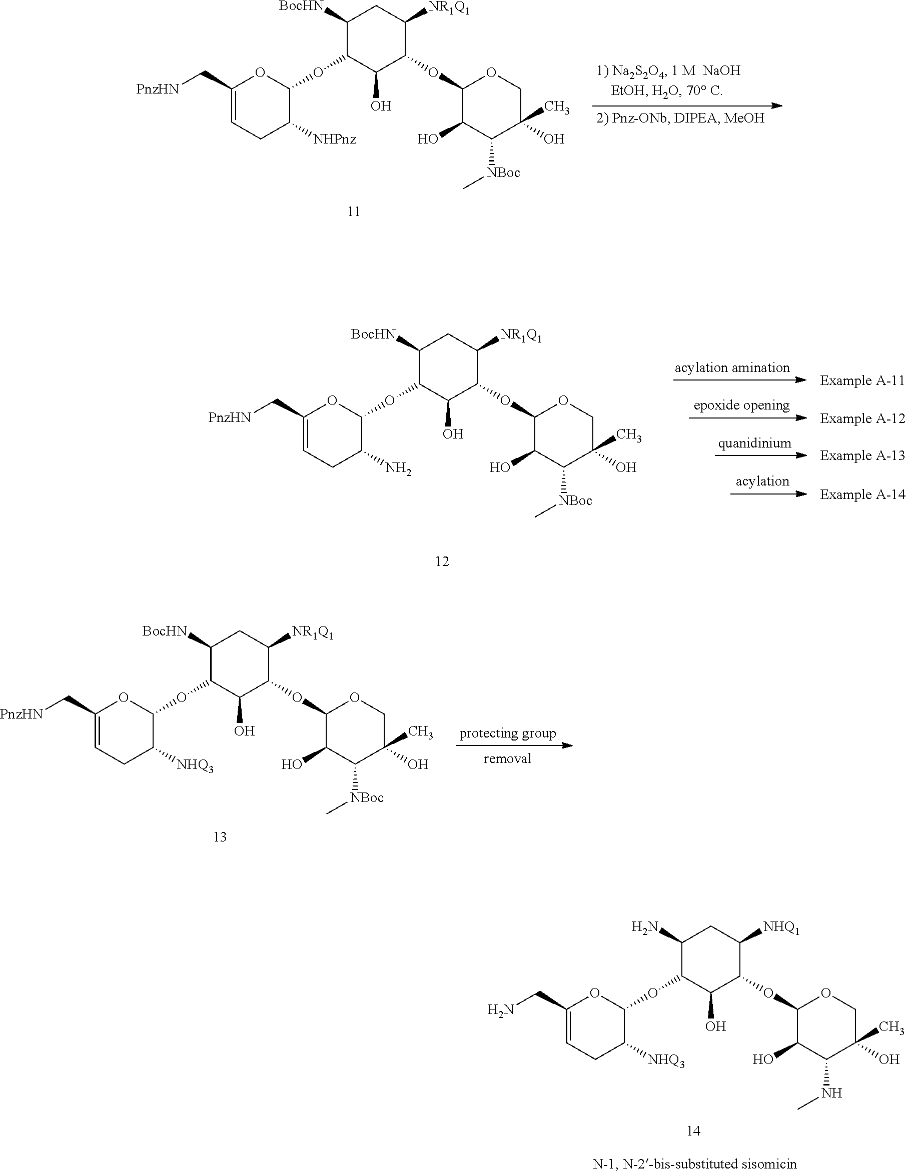

### Example A-1

### N-1 Acylation

### Method A:

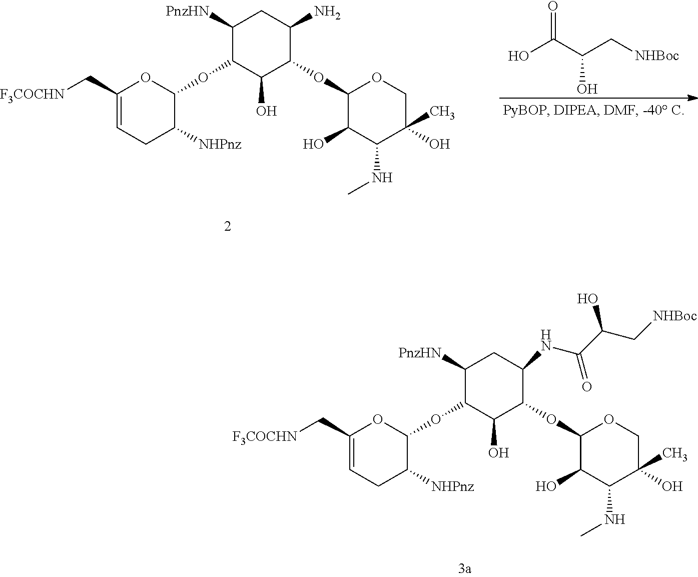

### Method B:

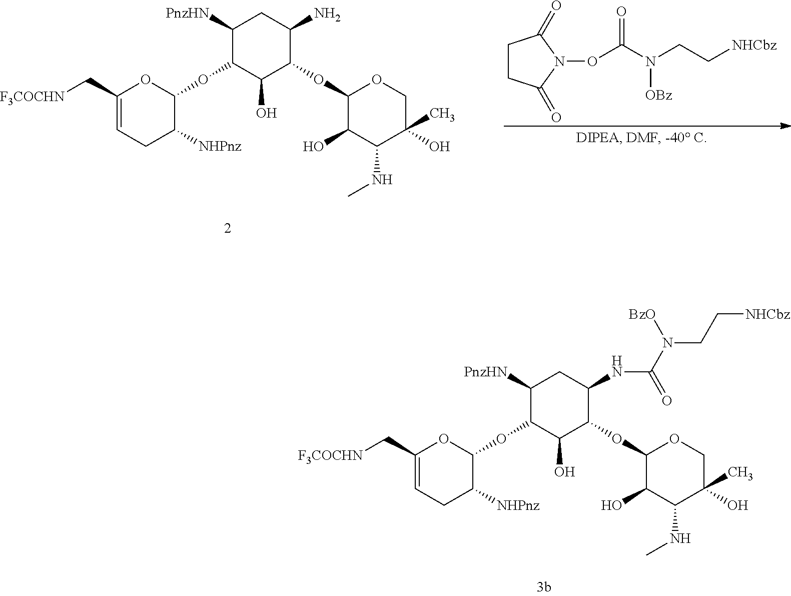

### Example A-2

### N-1 Epoxide Opening

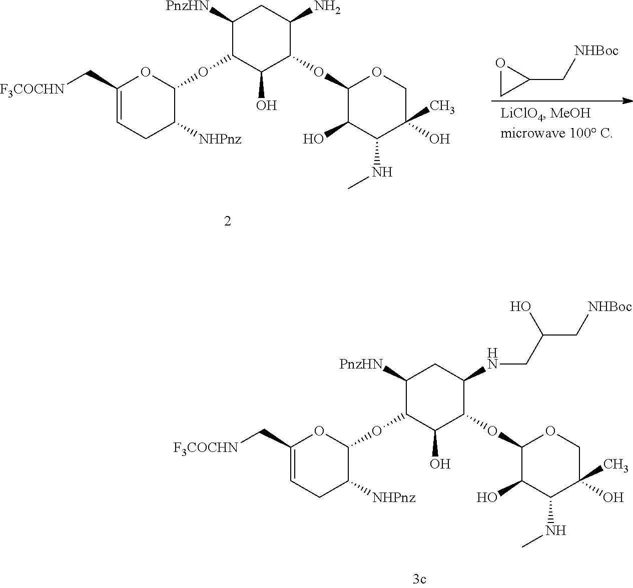

### Example A-3

### N-1 Sulfonylation

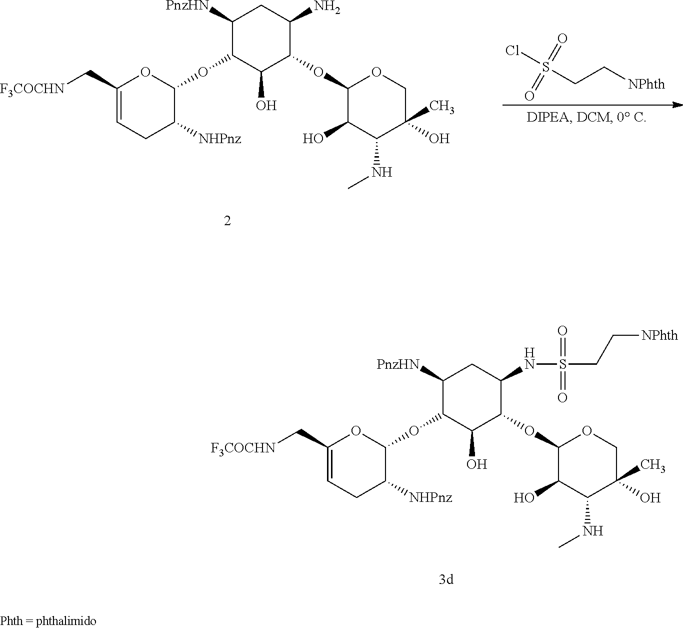

### Example A-4

### N-1 Reductive Amination

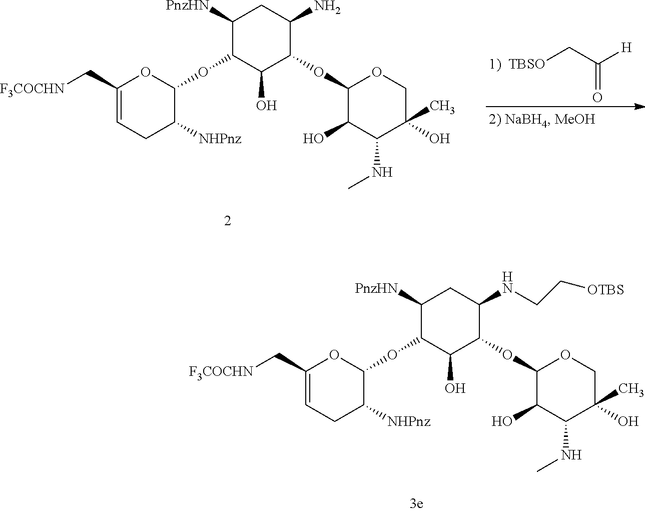

### Example A-5

### N-6′ Reductive Amination

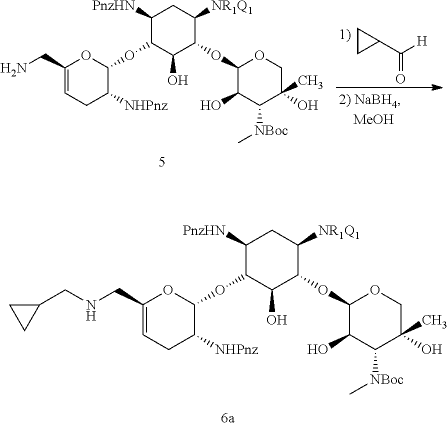

### Example A-6

### N-6′ Epoxide Opening

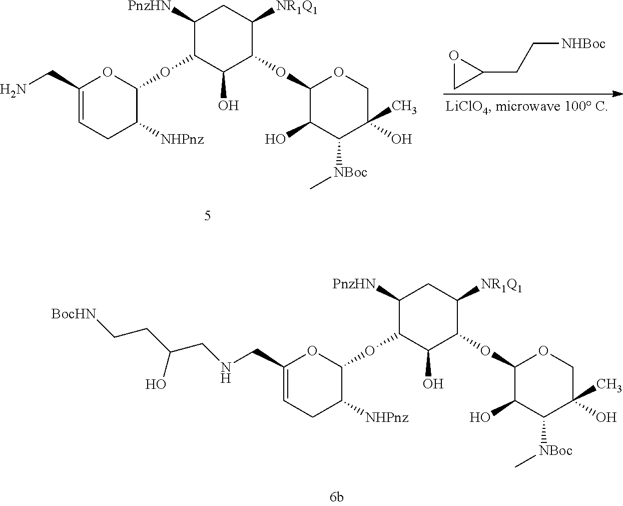

### Example A-7

### N-1 Acylation

### Method A:

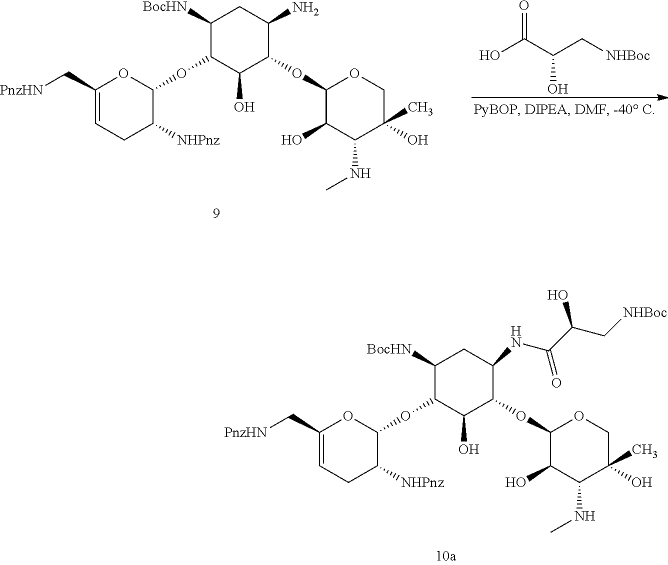

### Method B:

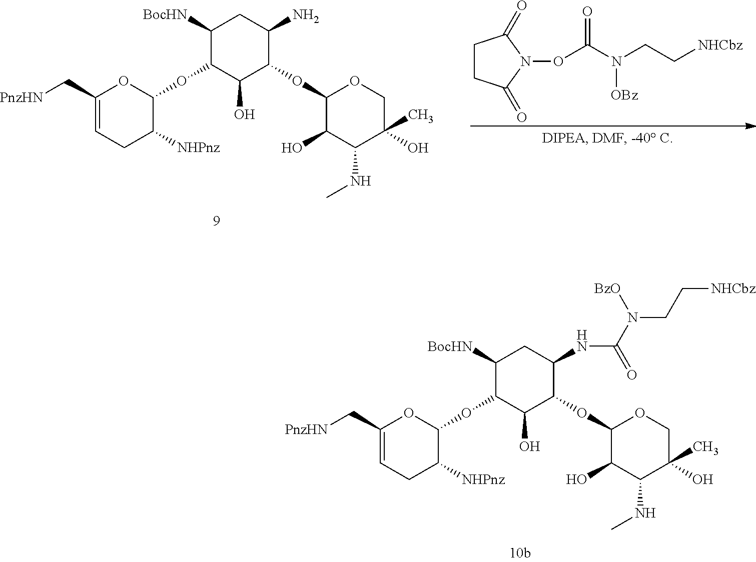

### Example A-8

### N-1 Epoxide Opening

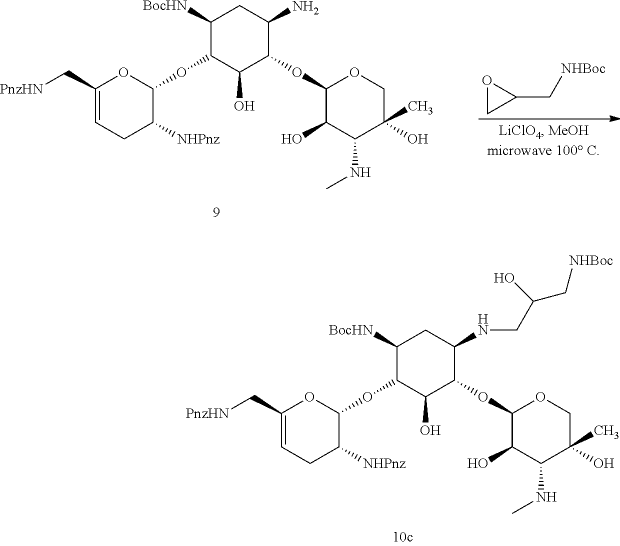

### Example A-9

### N-1 Sulfonylation

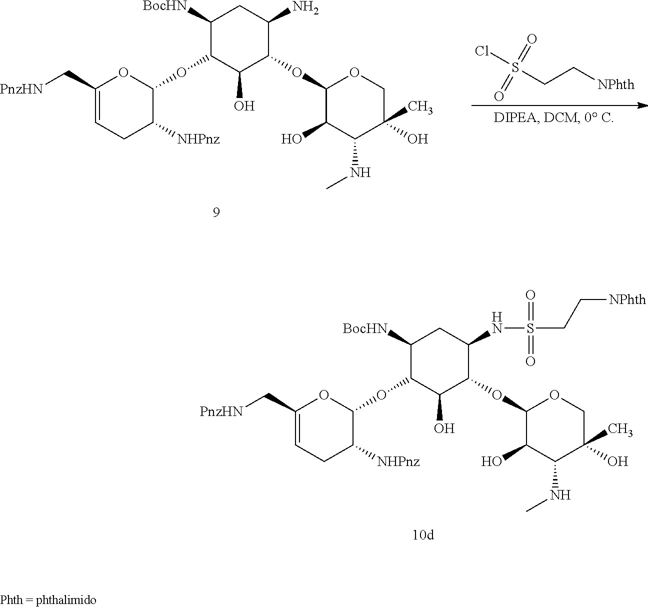

### Example A-10

### N-1 Reductive Amination

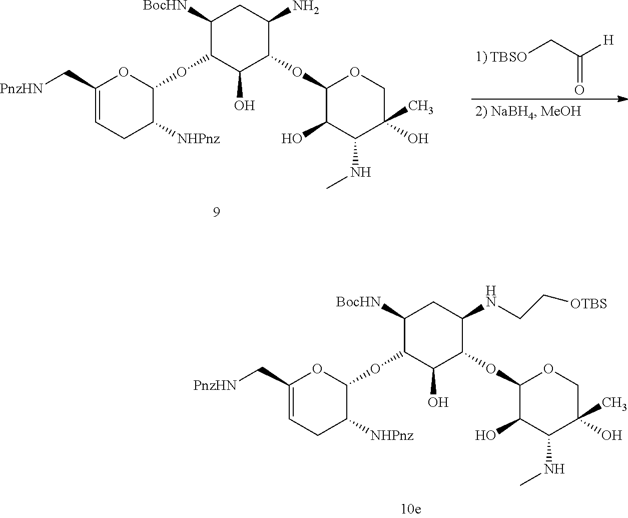

### Example A-11

### N-2′ Reductive Amination

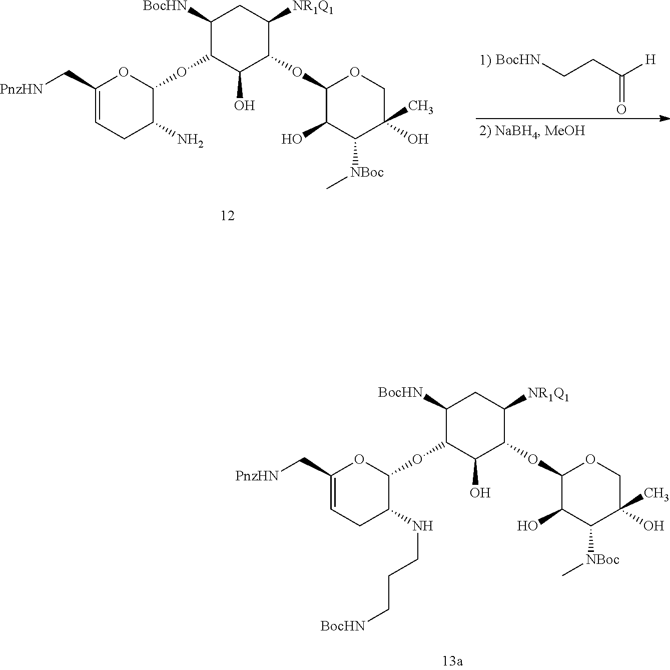

### Example A-12

### N-2′ Epoxide Opening

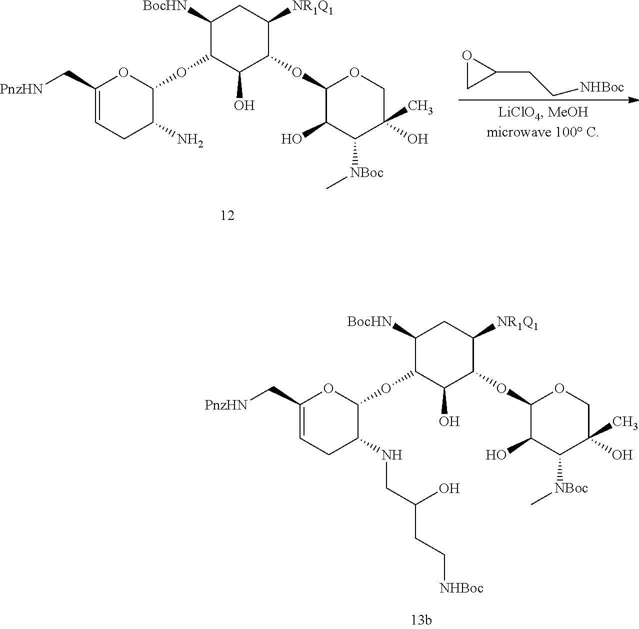

### Example A-13

### N-2′ Guanidinium

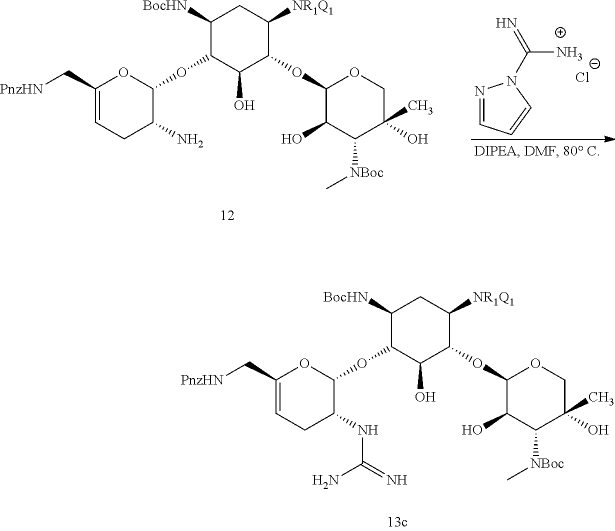

### Example A-14

### N-2′ Acylation

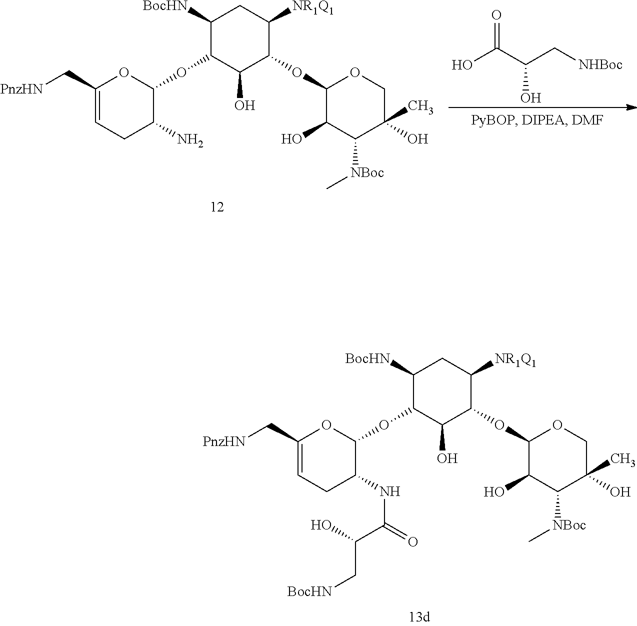

Kanamycin B Analogs

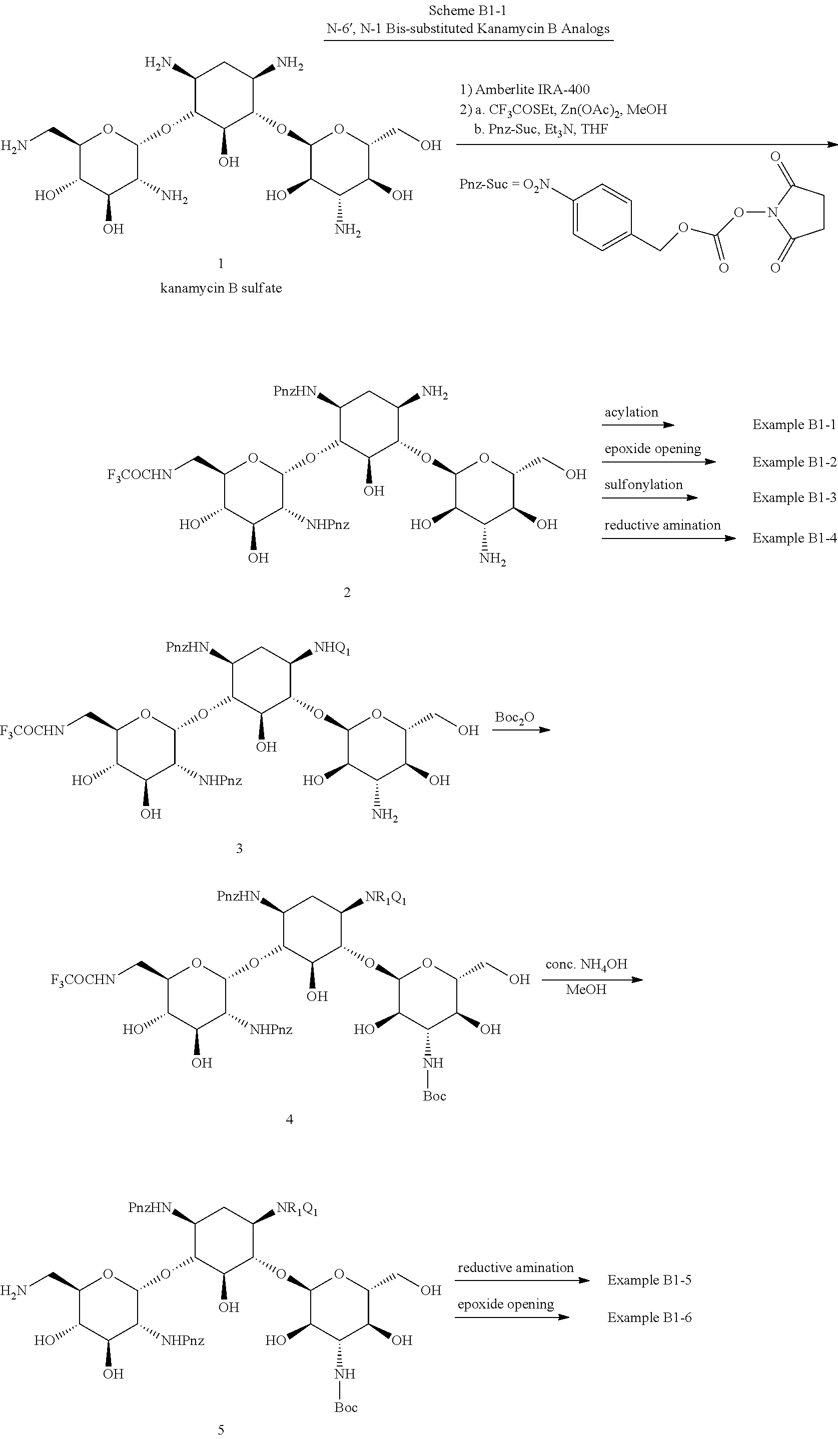

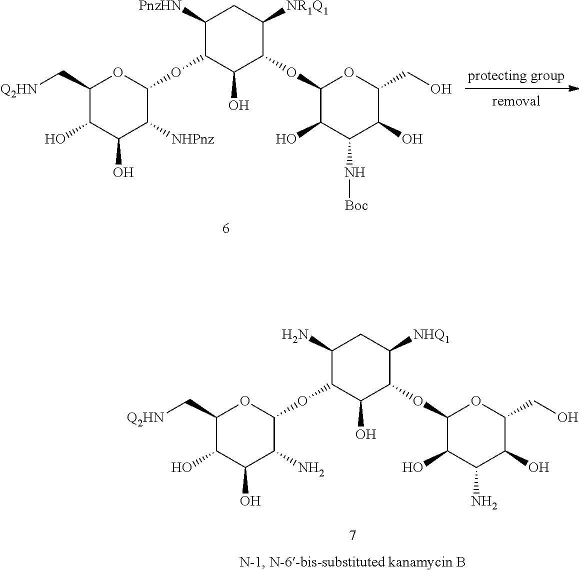

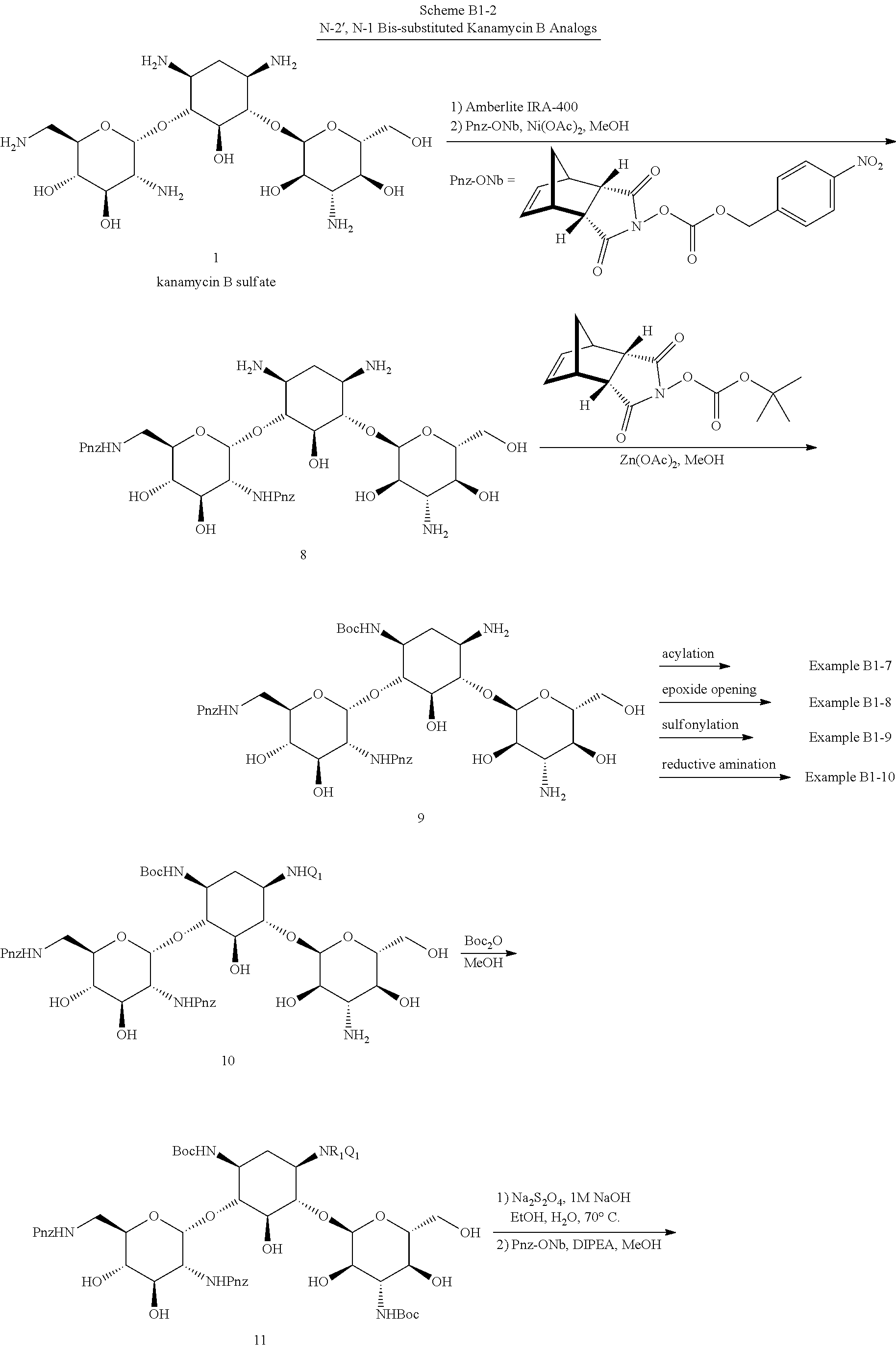

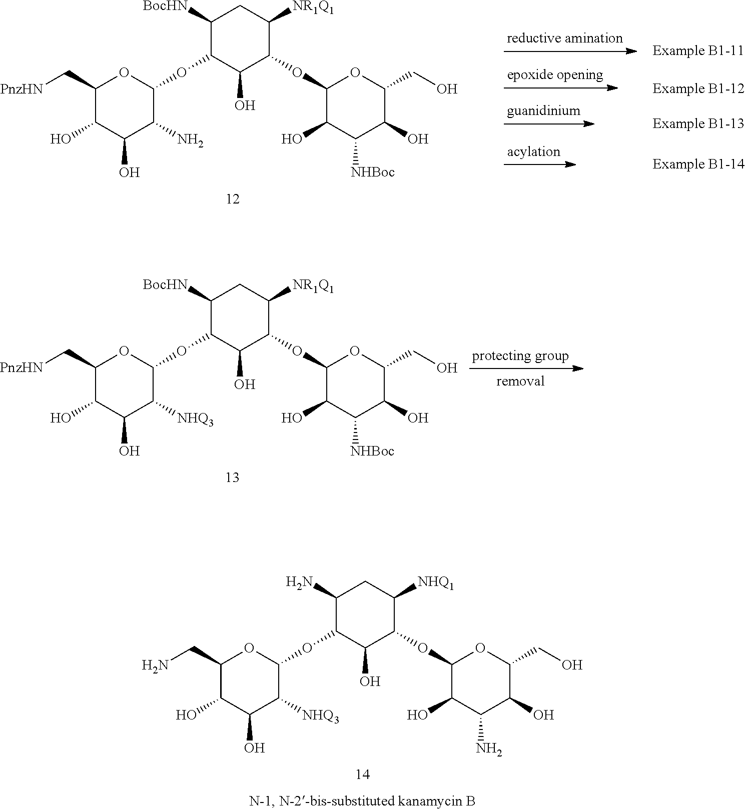

### Example B1-1

### N-1 Acylation

### Method A:

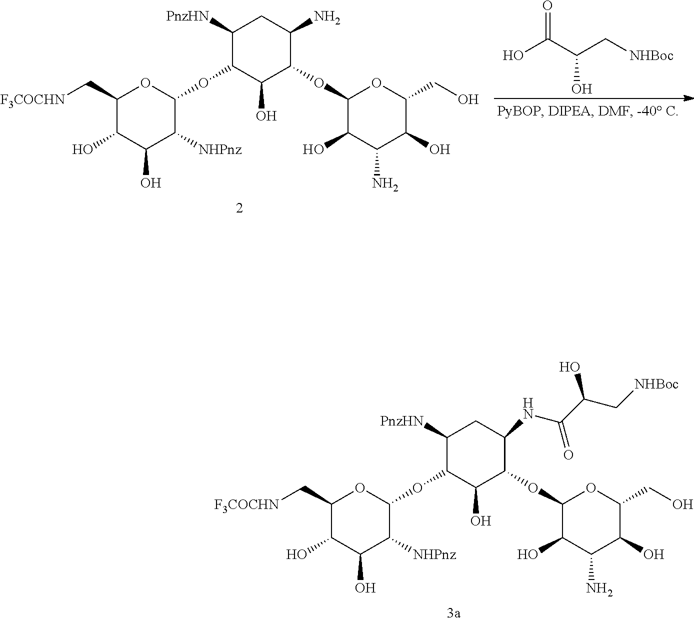

### Method B:

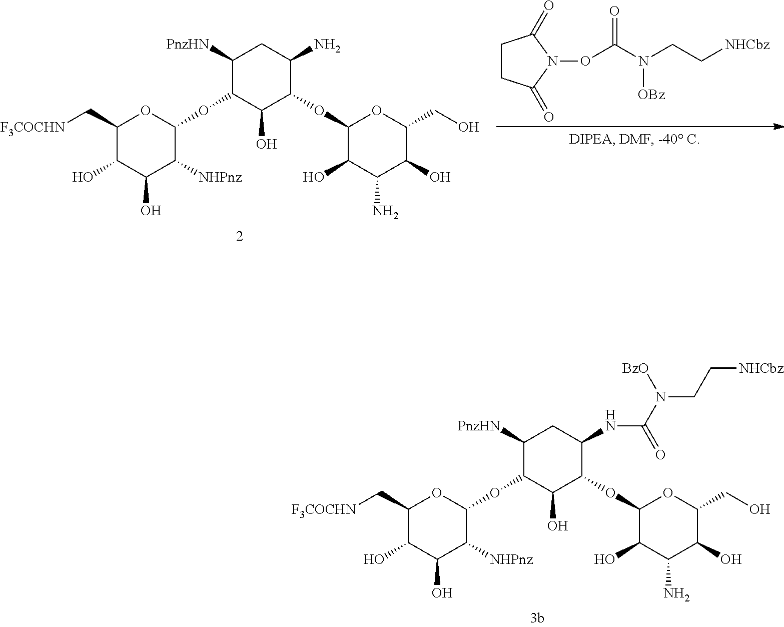

### Example B1-2

### N-1 Epoxide Opening

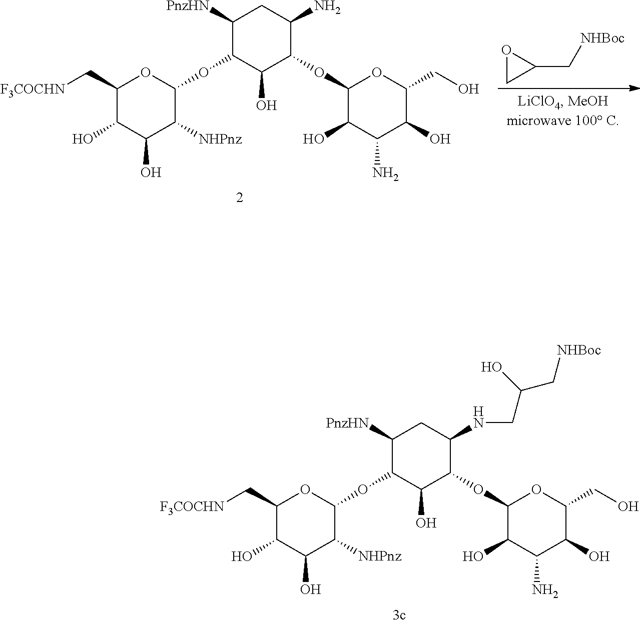

### Example B1-3

### N-1 Sulfonylation

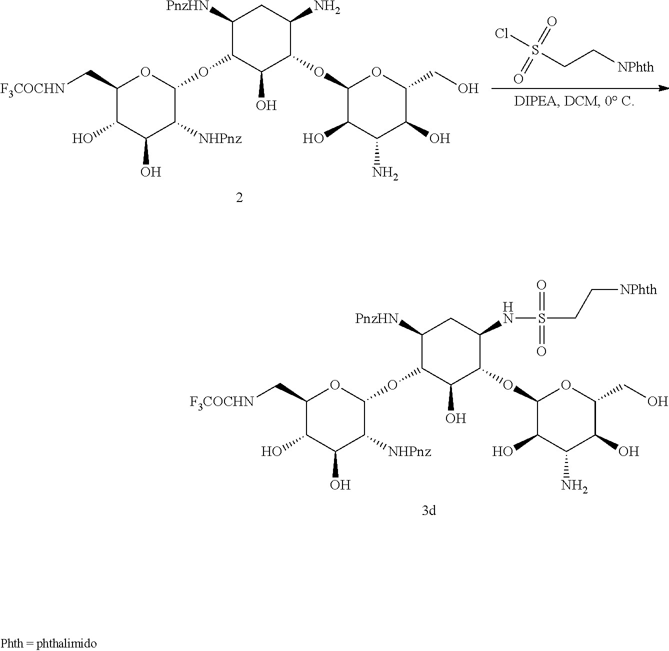

### Example B1-4

### N-1 Reductive Amination

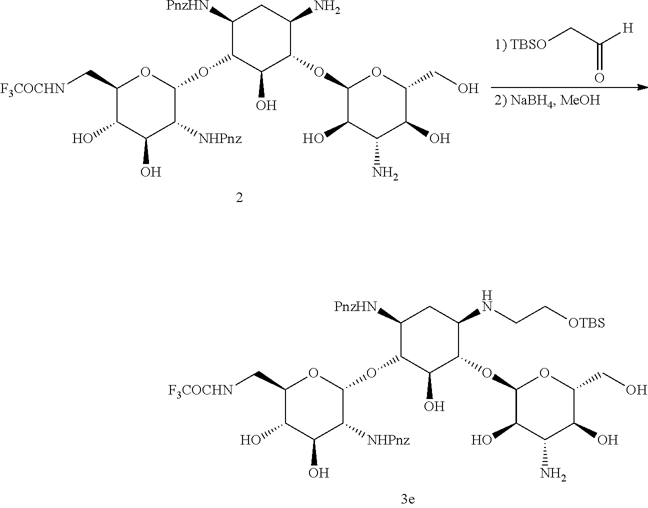

### Example B1-5

### N-6′ Reductive Amination

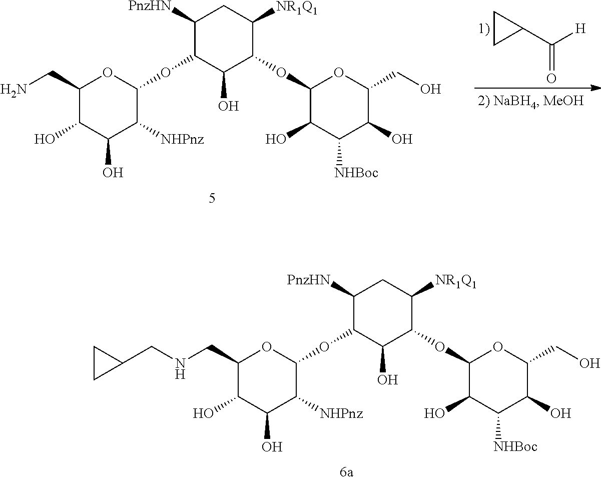

### Example B1-6

### N-6′ Epoxide Opening

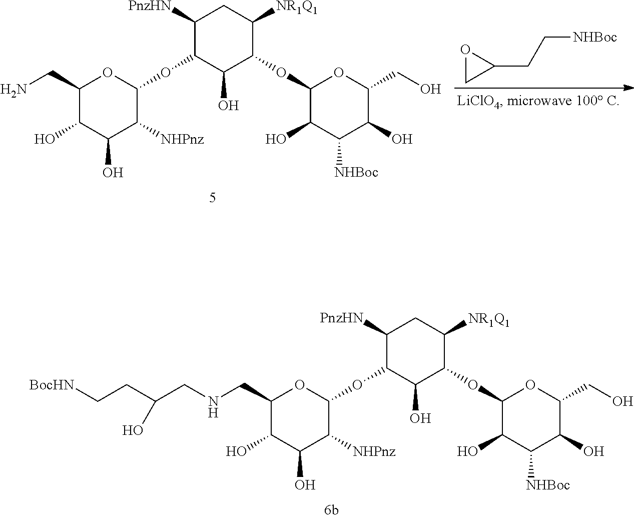

### Example B1-7

### N-1 Acylation

### Method A:

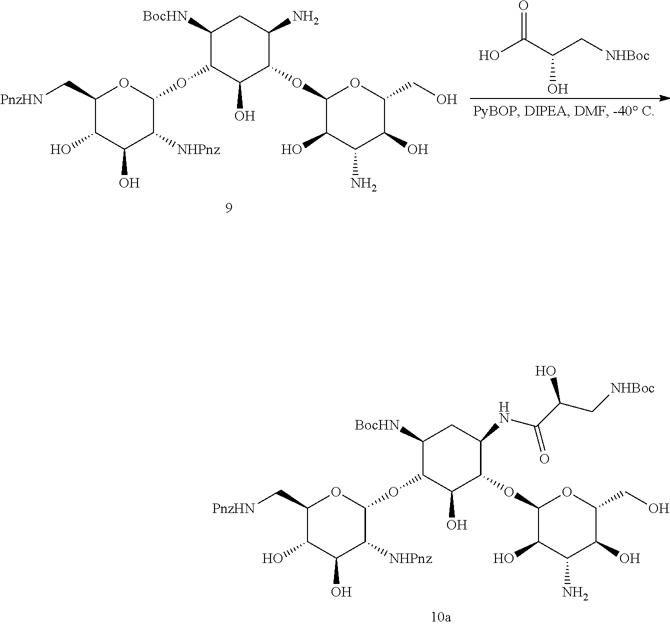

### Method B:

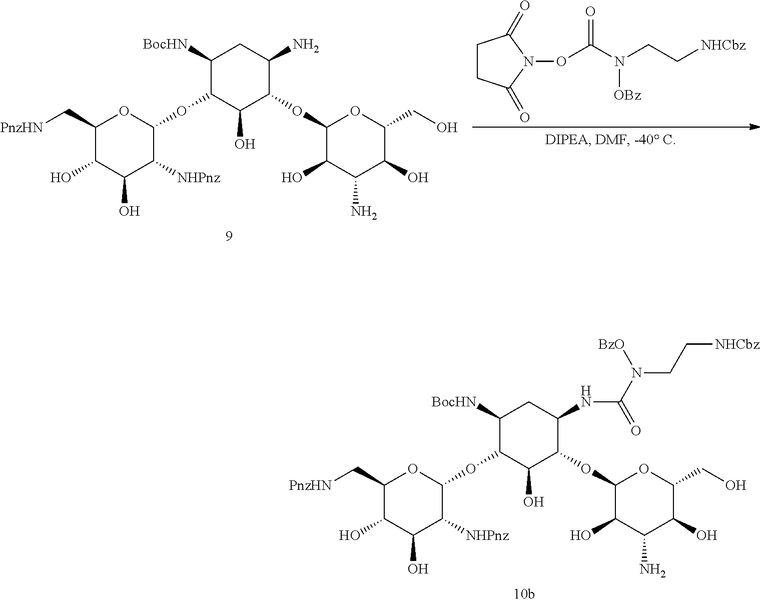

### Example B1-8

### N-1 Epoxide Opening

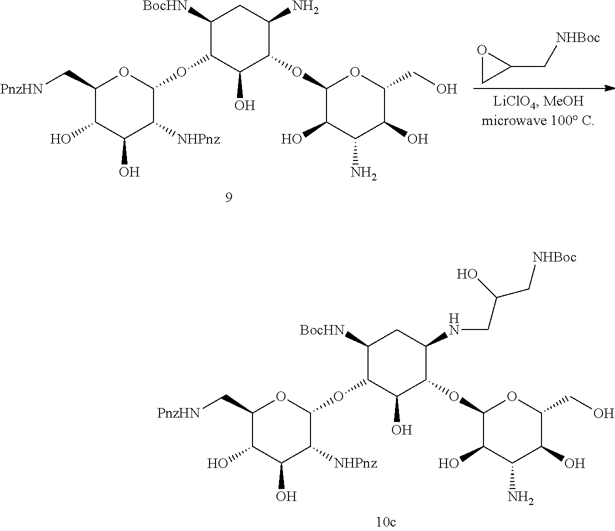

### Example B1-9

### N-1 Sulfonylation

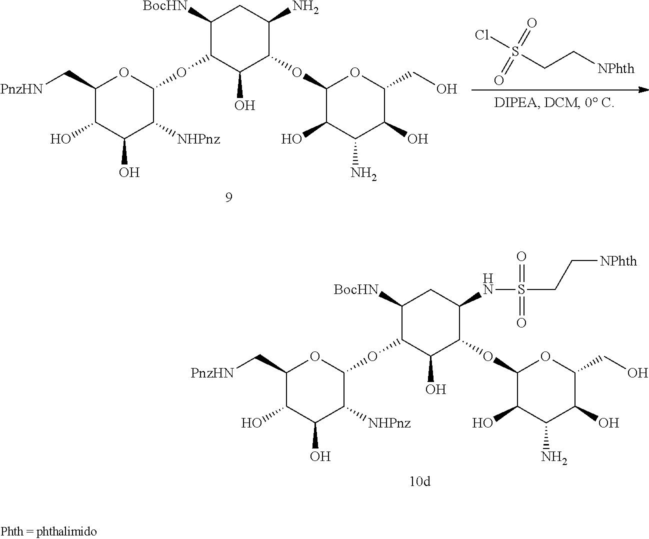

### Example B1-10

### N-1 Reductive Amination

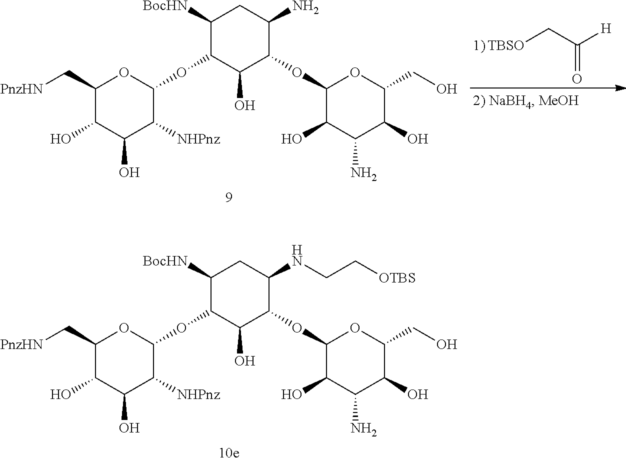

### Example B1-11

### N-2′ Reductive Amination

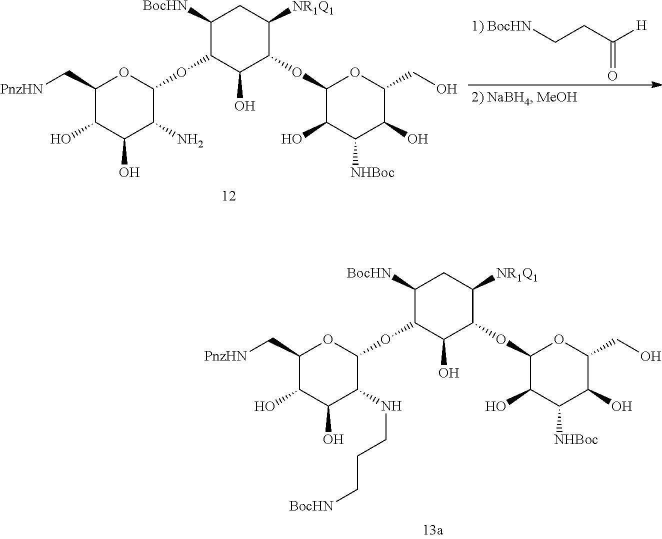

### Example B1-12

### N-2′ Epoxide Opening

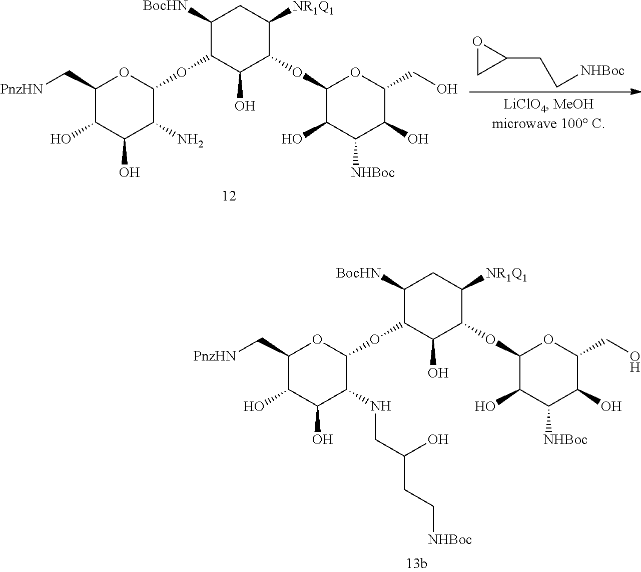

### Example B1-13

### N-2′ Guanidinium

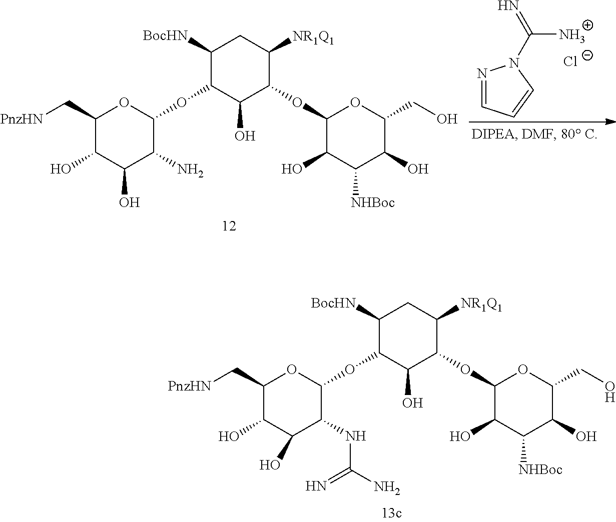

### Example B1-14

### N-2′ Acylation

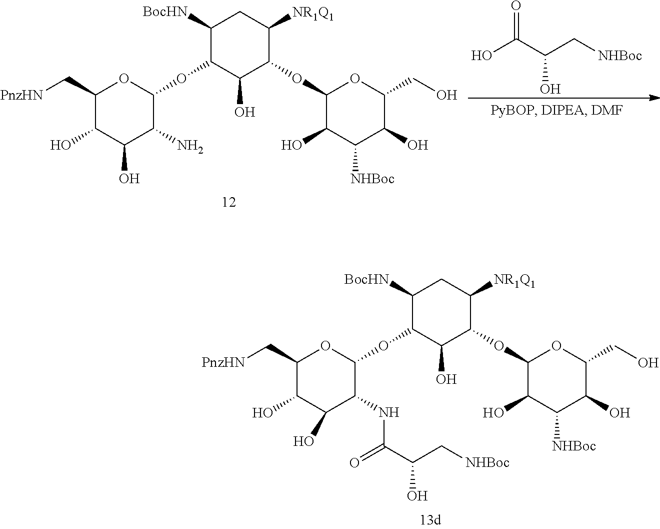

Kanamycin A Analogs

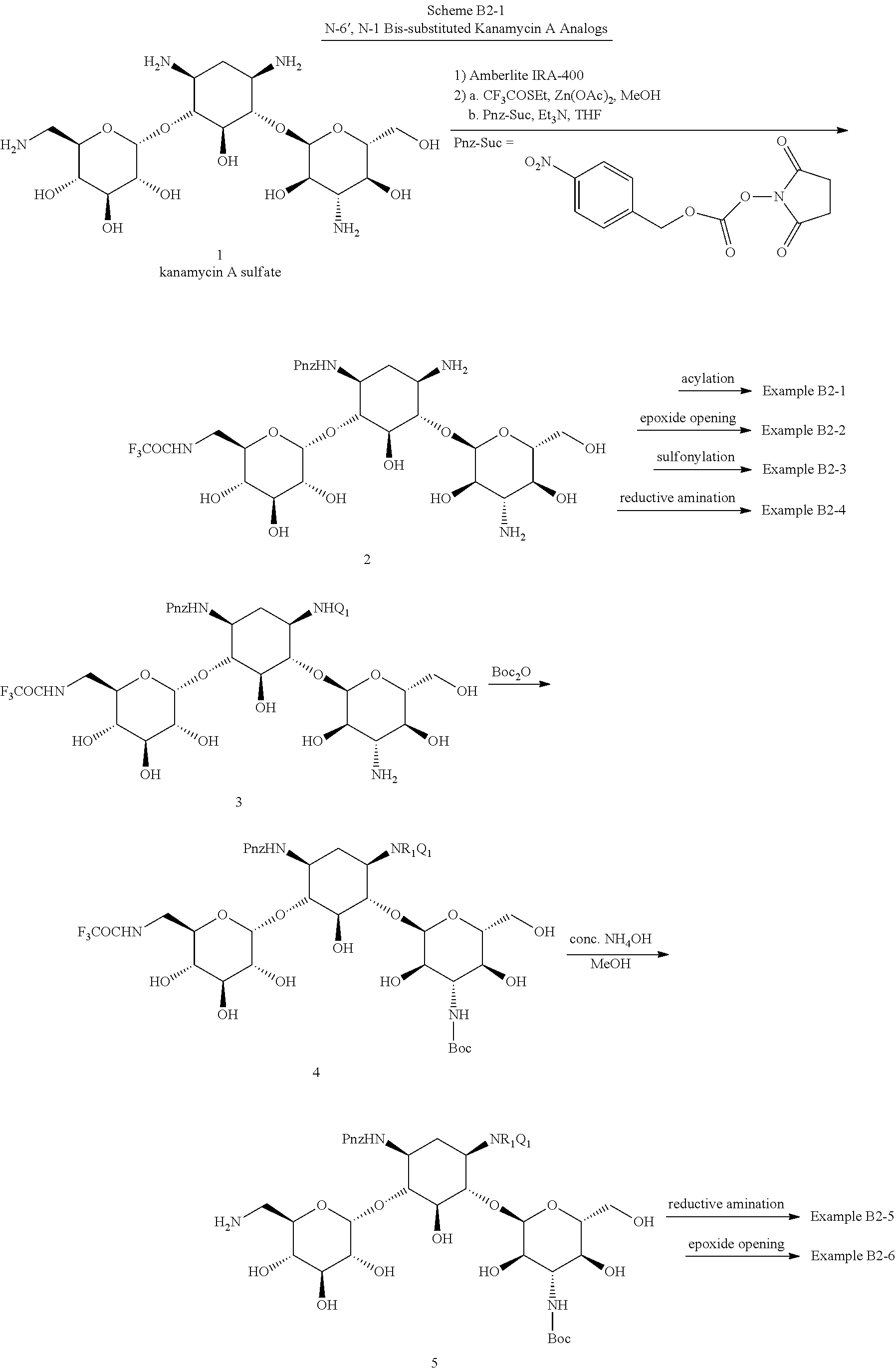

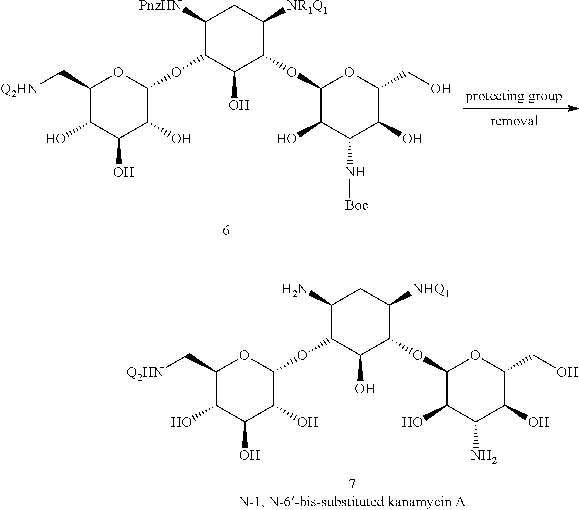

### Example B2-1

### N-1 Acylation

### Method A:

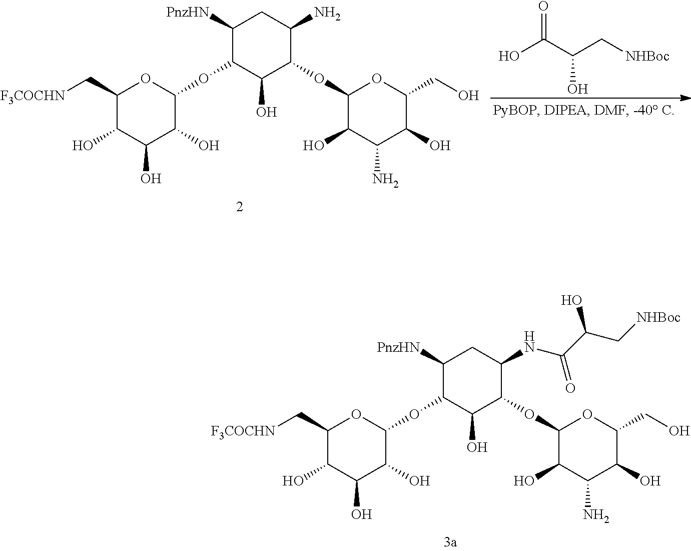

### Method B:

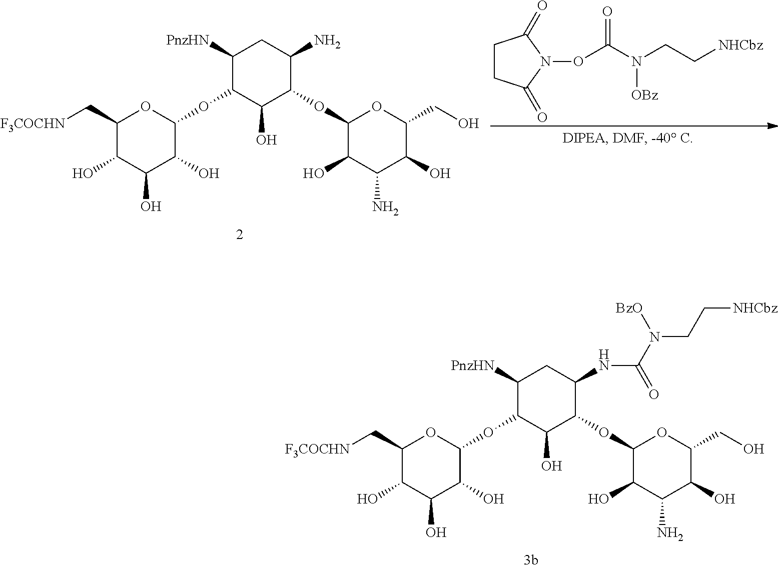

### Example B2-2

### N-1 Epoxide Opening

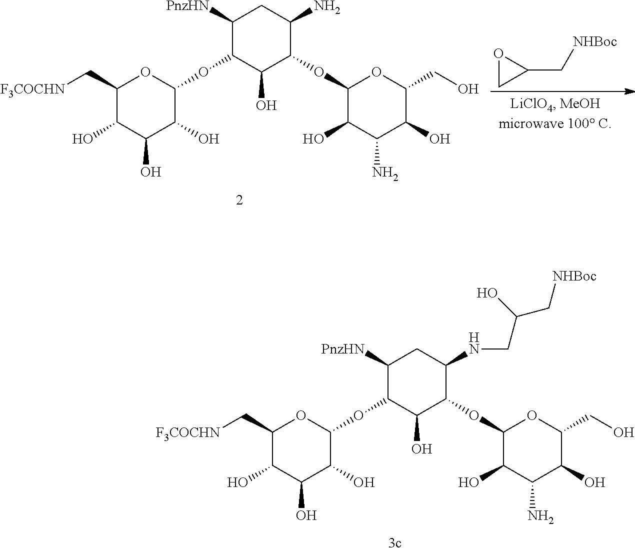

### Example B2-3

### N-1 Sulfonylation

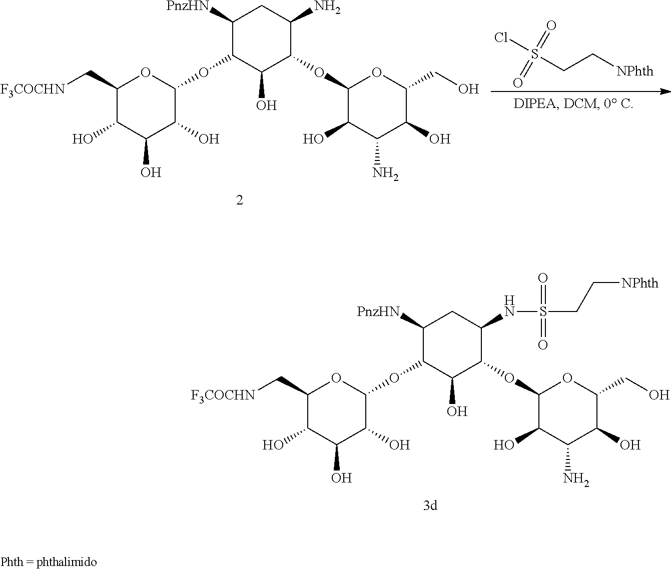

### Example B2-4

### N-1 Reductive Amination

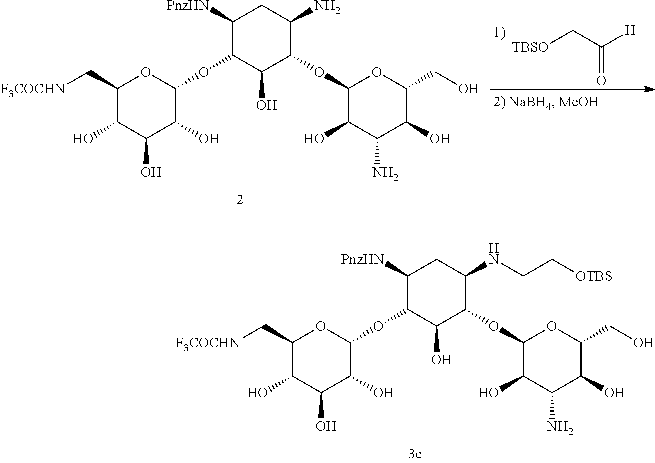

### Example B2-5

### N-6′ Reductive Amination

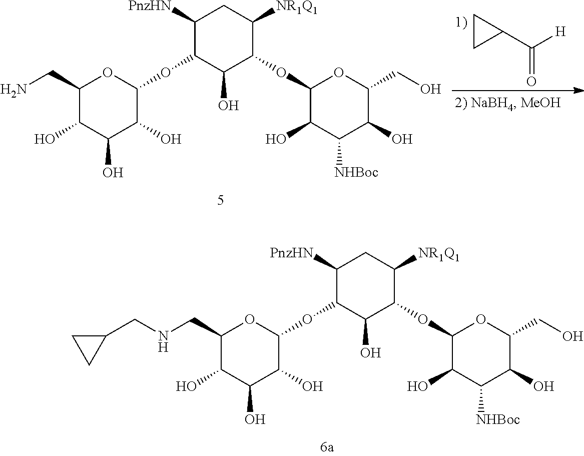

### Example B2-6

### N-6′ Epoxide Opening

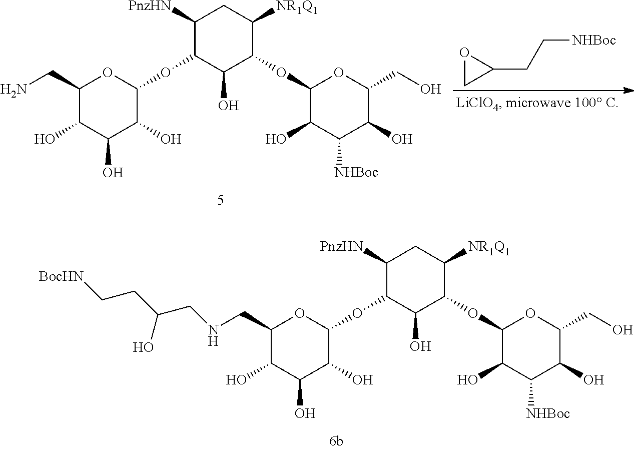

Dibekacin Analogs

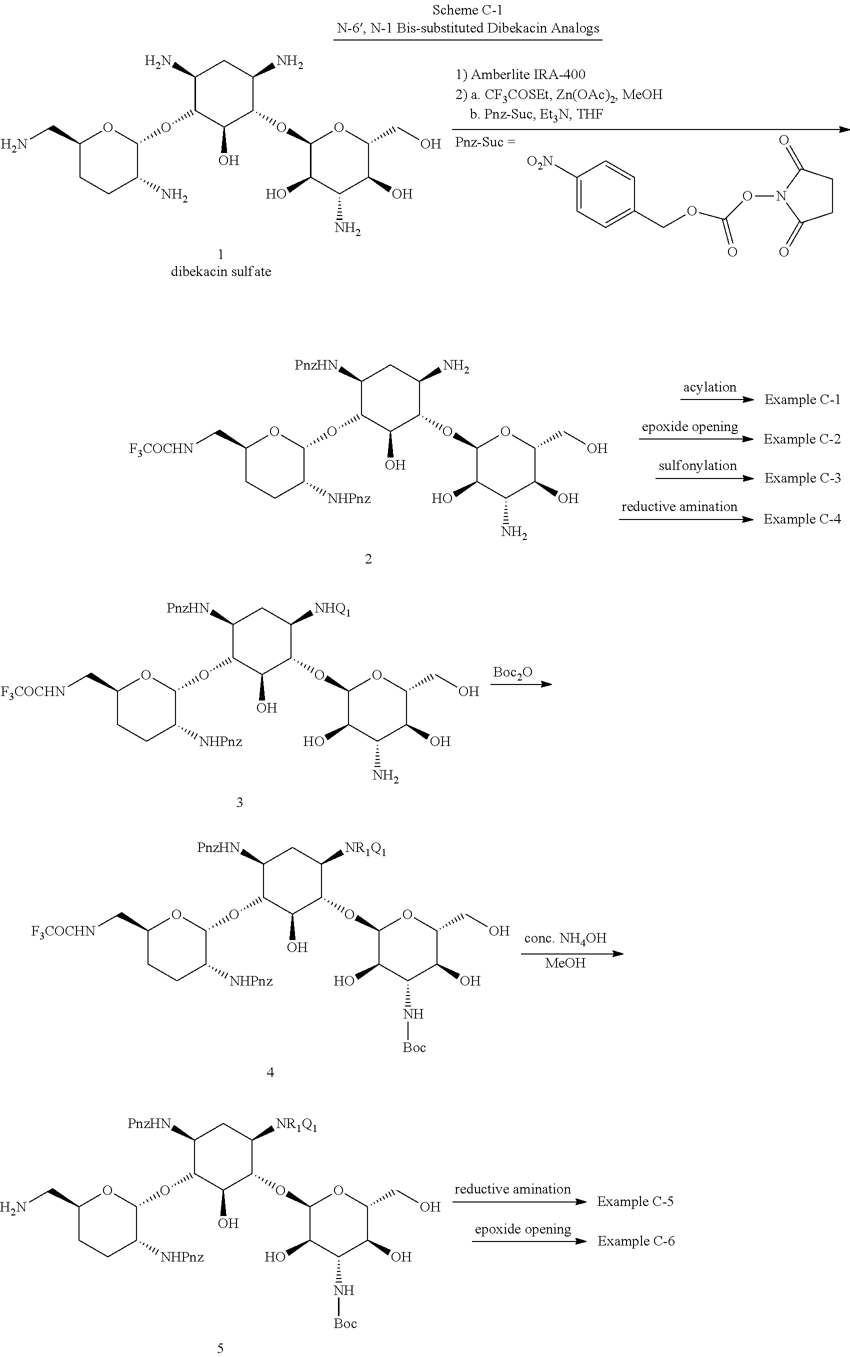

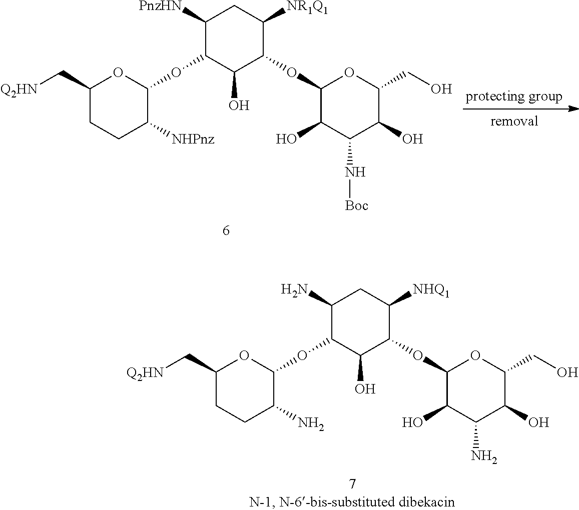

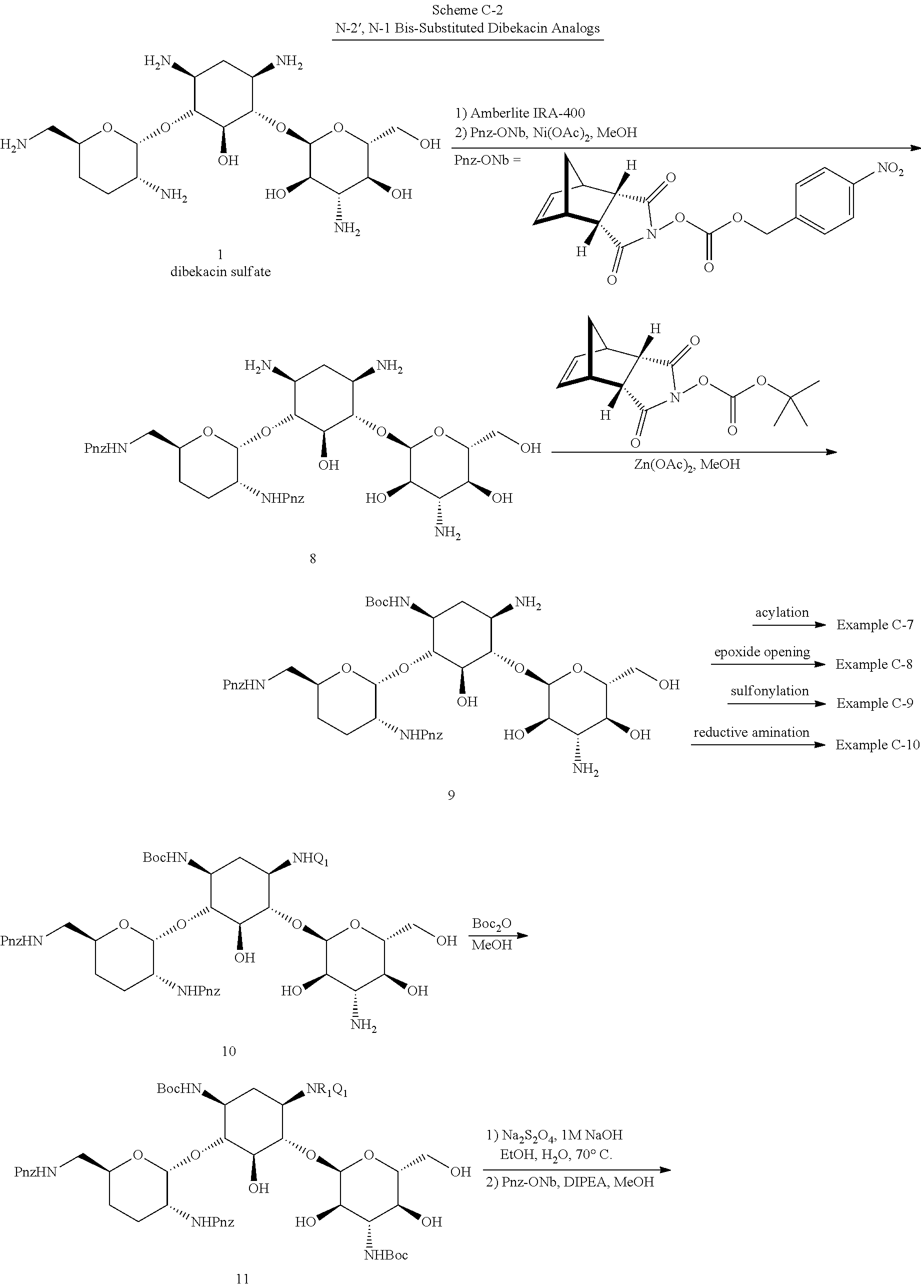

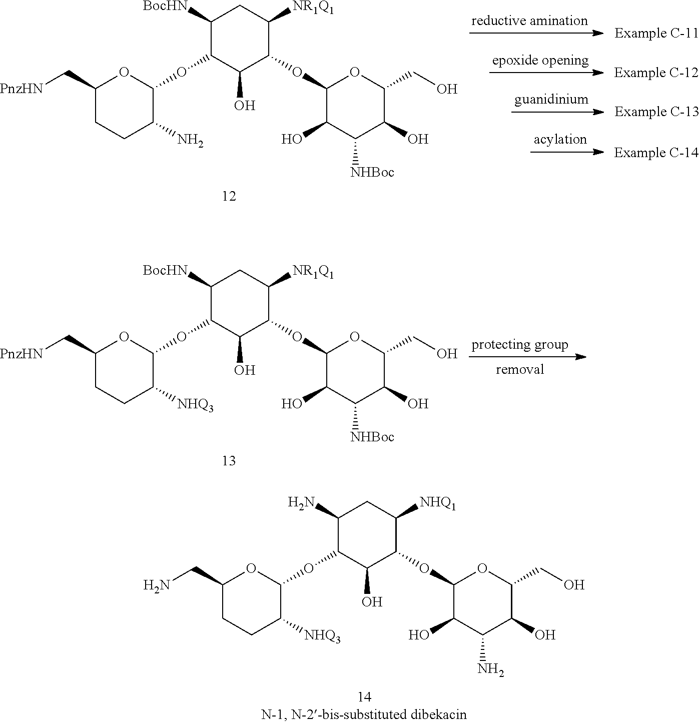

### Example C-1

### N-1 Acylation

### Method A:

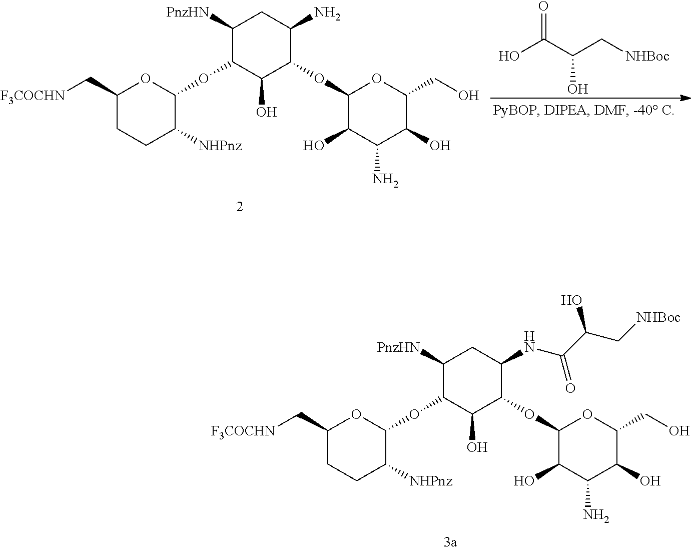

### Method B:

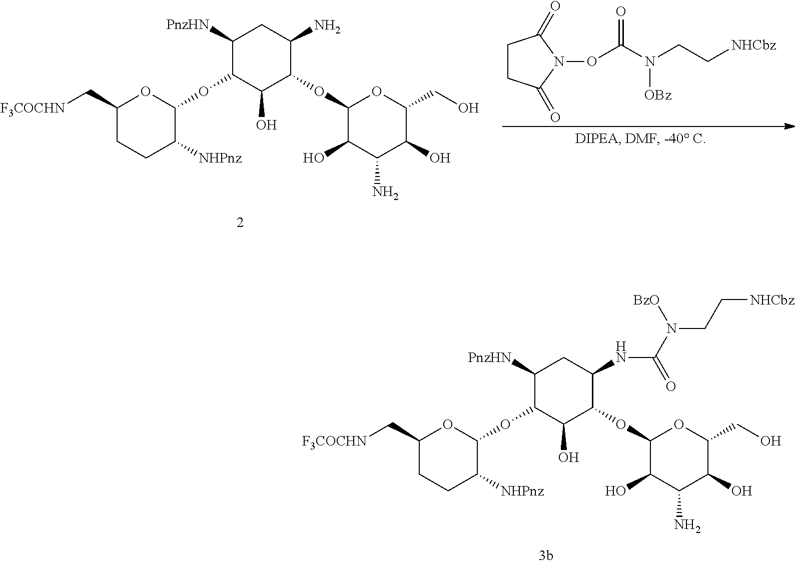

### Example C-2

### N-1 Epoxide Opening

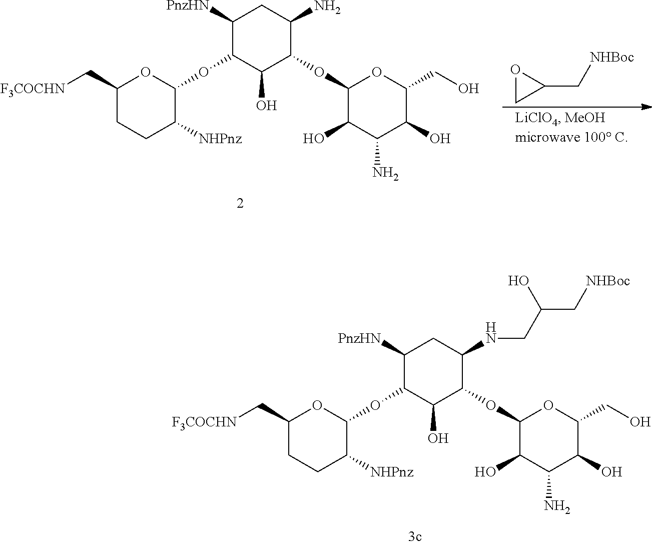

### Example C-3

### N-1 Sulfonylation

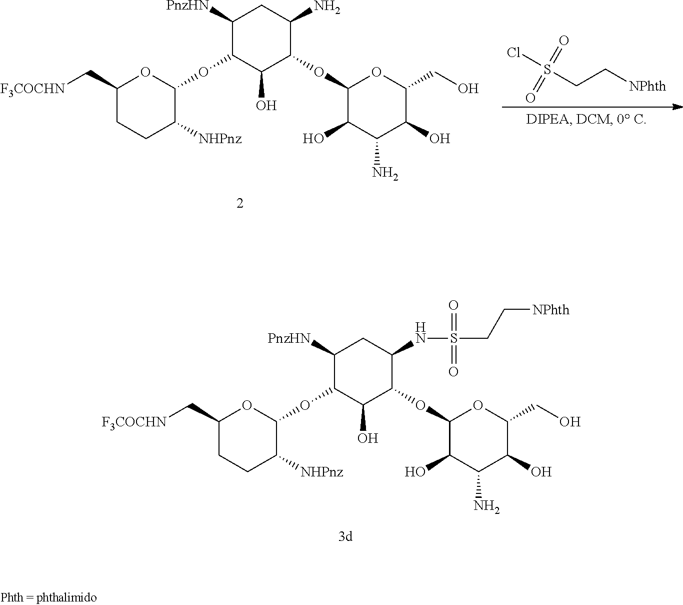

### Example C-4

### N-1 Reductive Amination

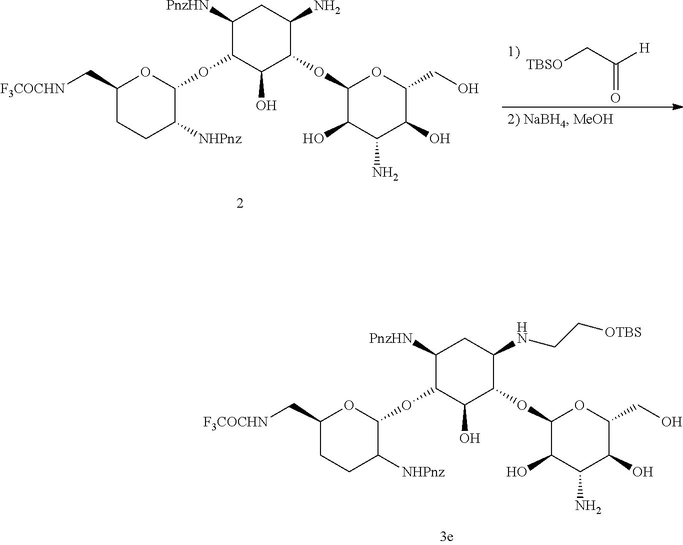

### Example C-5

### N-6′ Reductive Amination

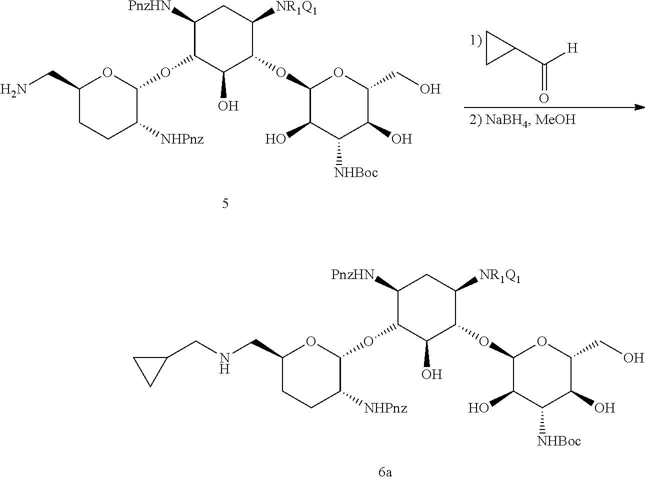

### Example C-6

### N-6′ Epoxide Opening

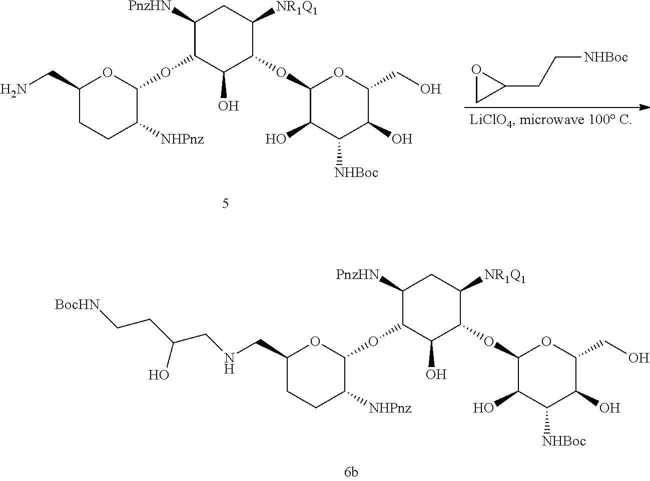

### Example C-7

### N-1 Acylation

### Method A:

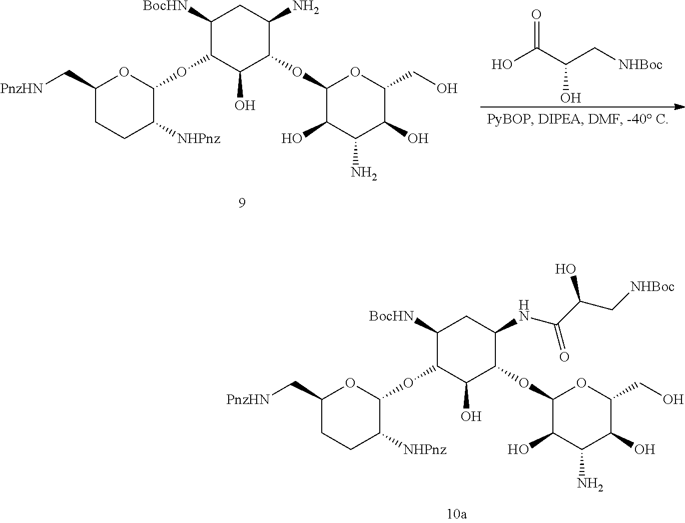

### Method B:

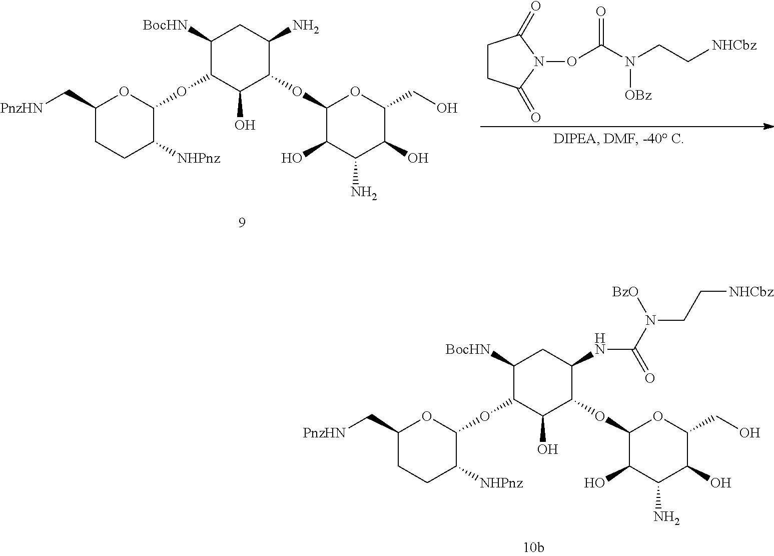

### Example C-8

### N-1 Epoxide Opening

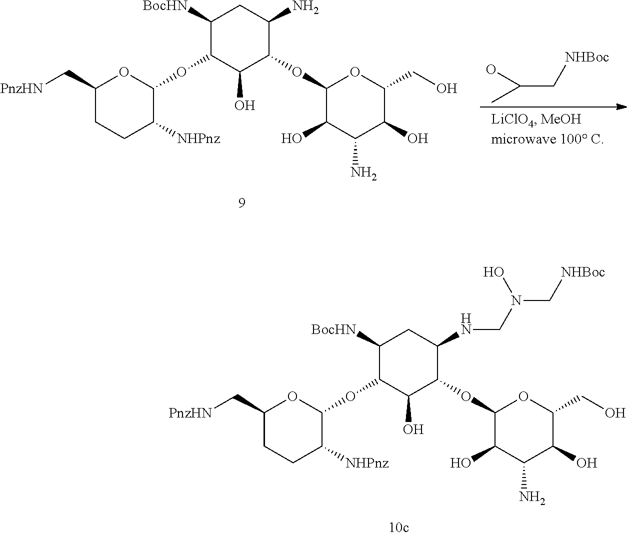

### Example C-9

### N-1 Sulfonylation

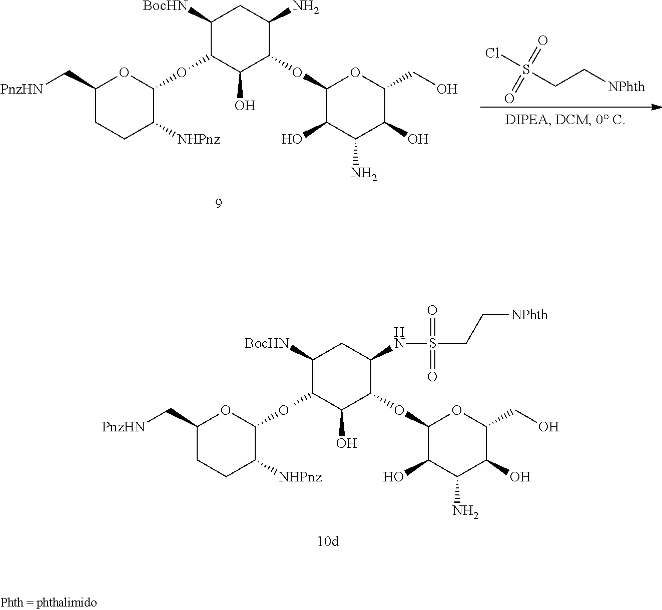

### Example C-10

### N-1 Reductive Amination

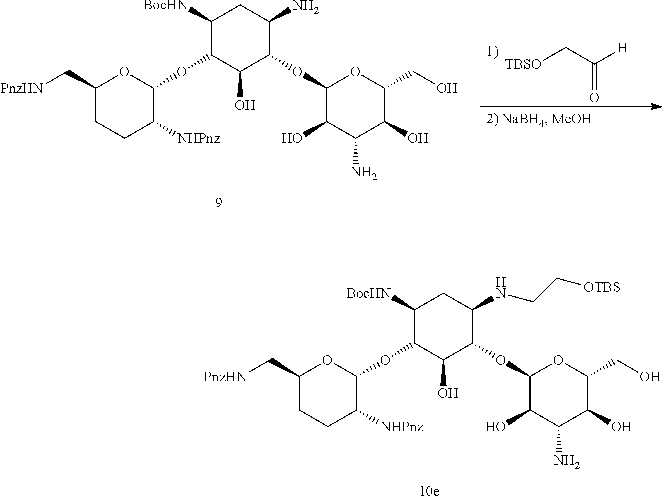

### Example C-11

### N-2′ Reductive Amination

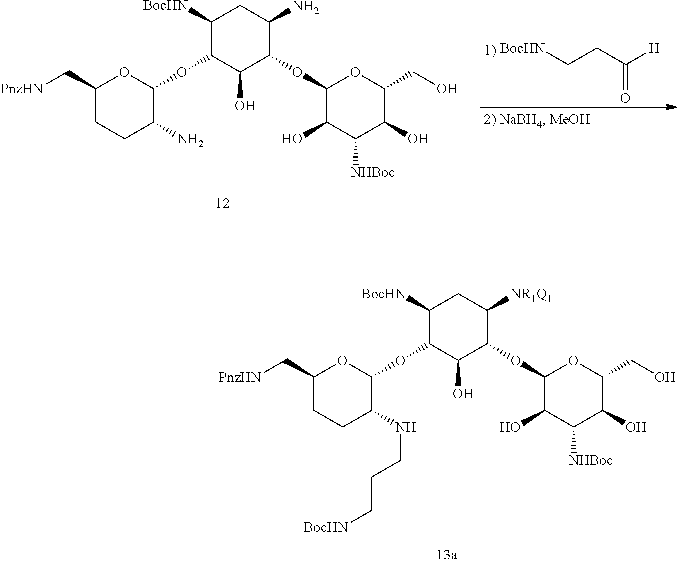

### Example C-12

### N-2′ Epoxide Opening

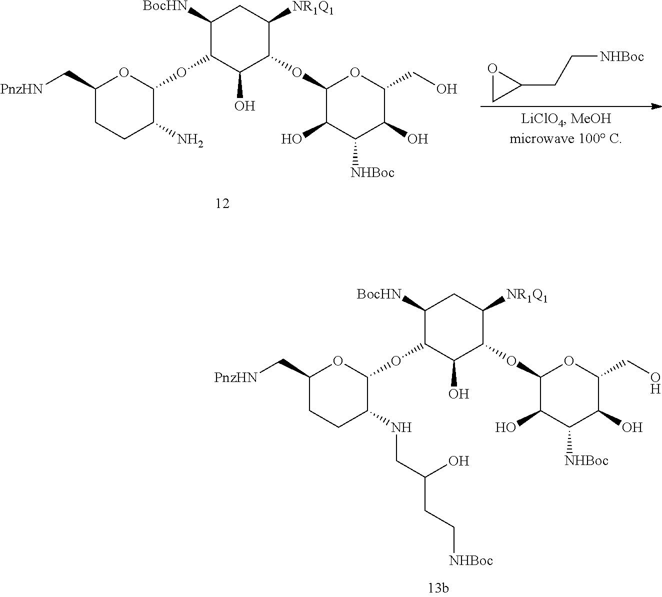

### Example C-13

N-2′ Guanidinium

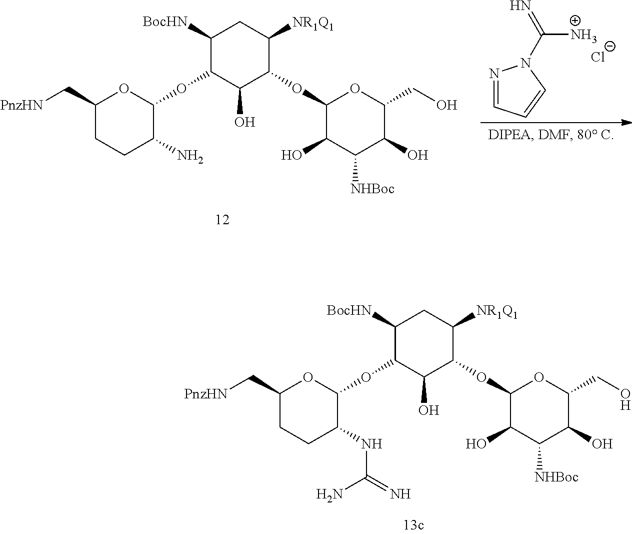

### Example C-14

### N-2′ Acylation

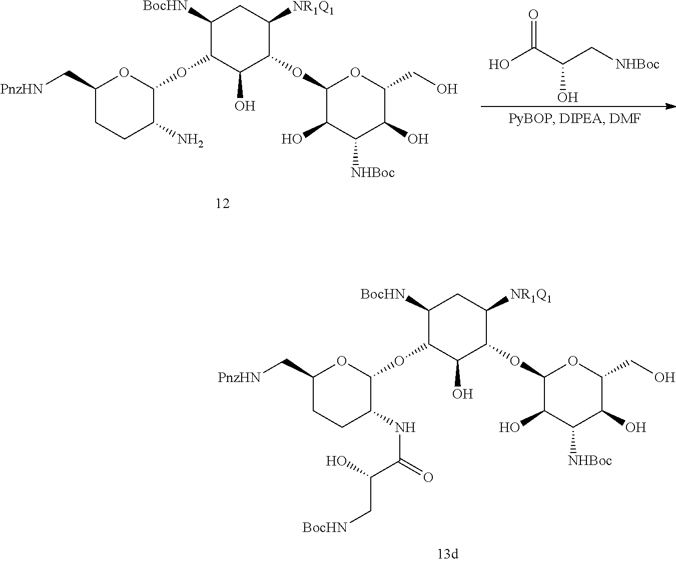

Tobramycin Analogs

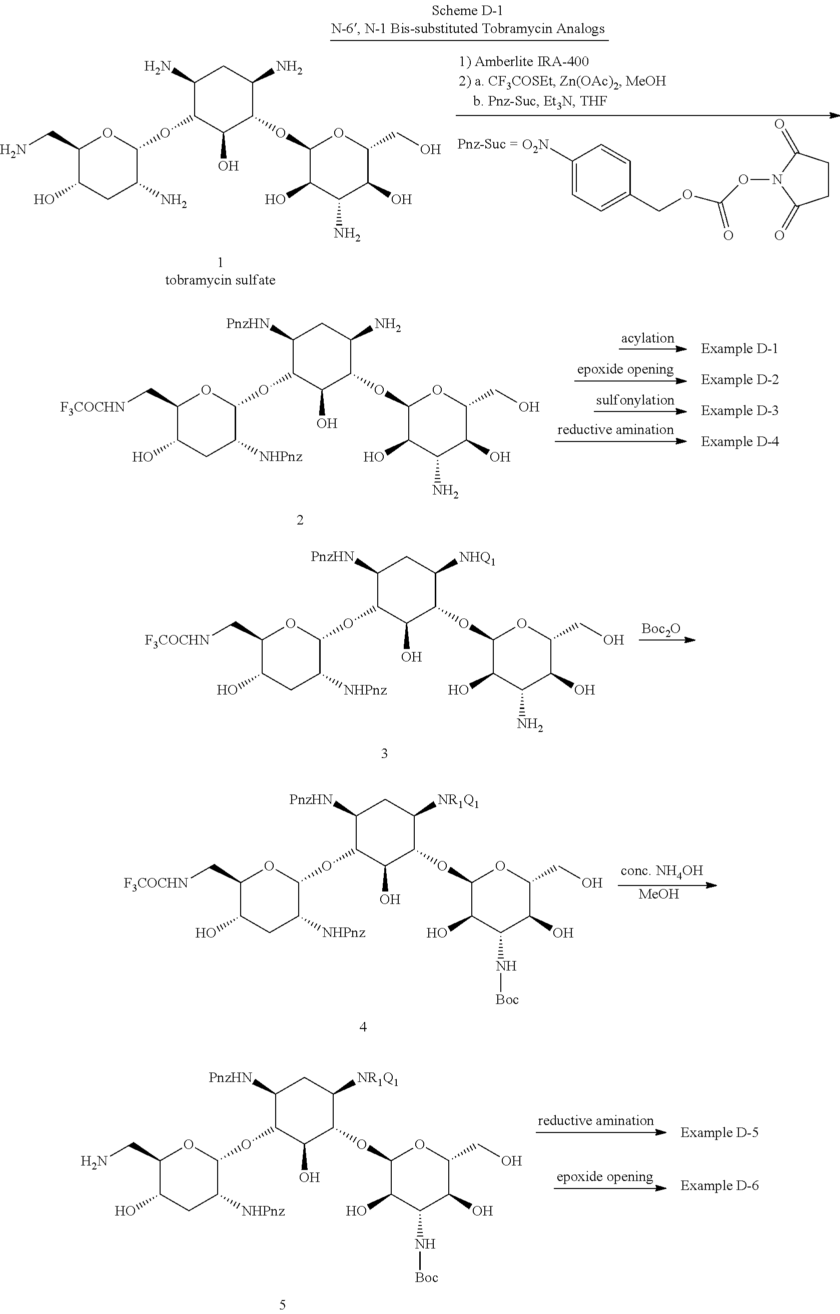

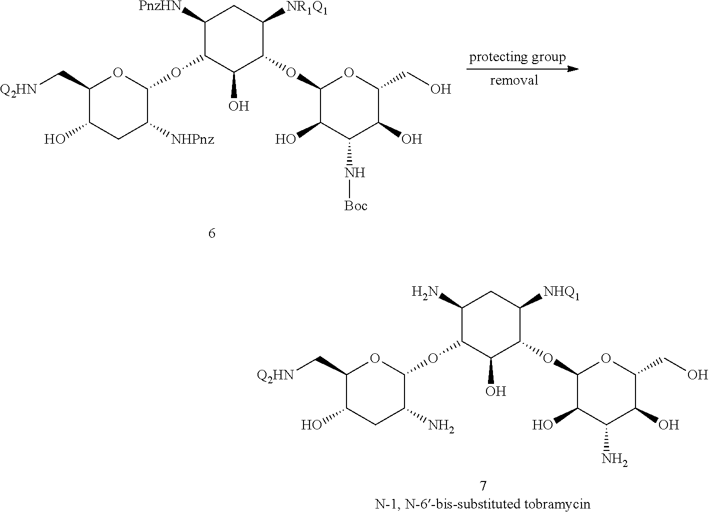

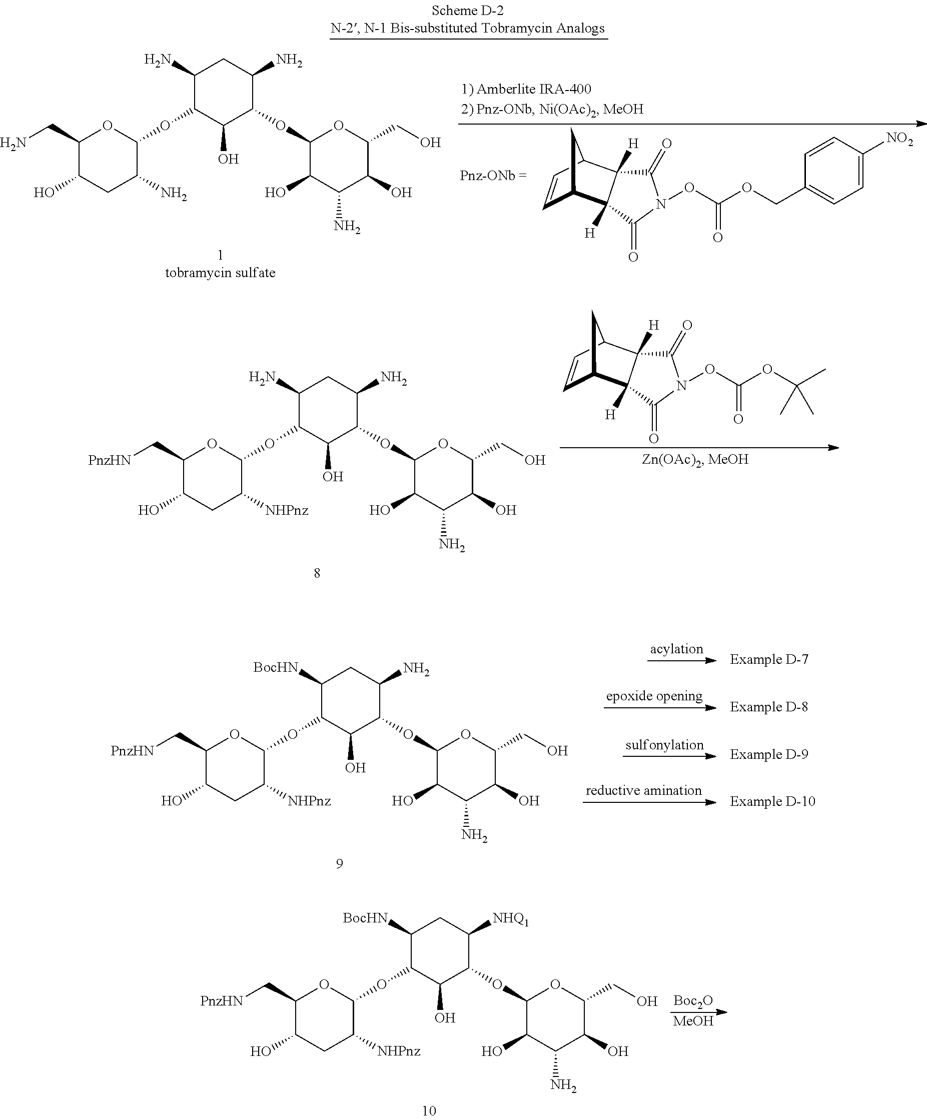

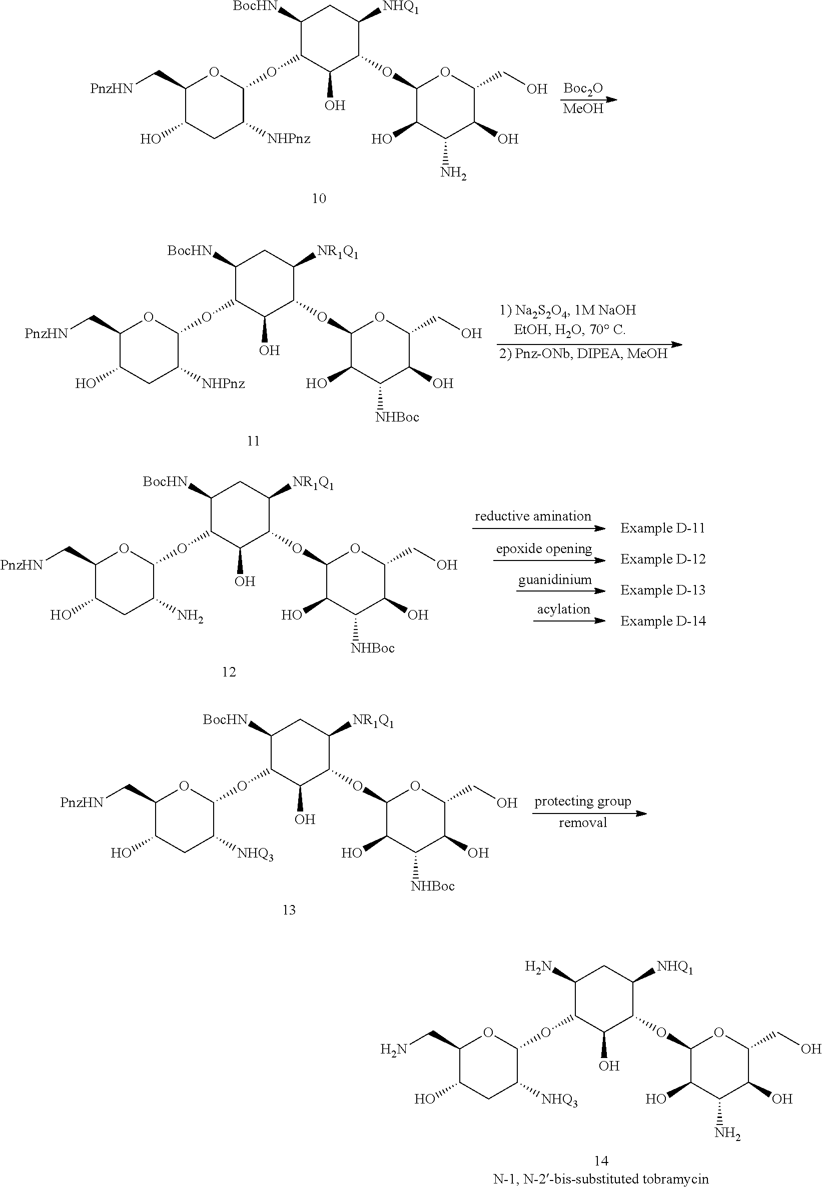

### Example D-1

### N-1 Acylation

### Method A:

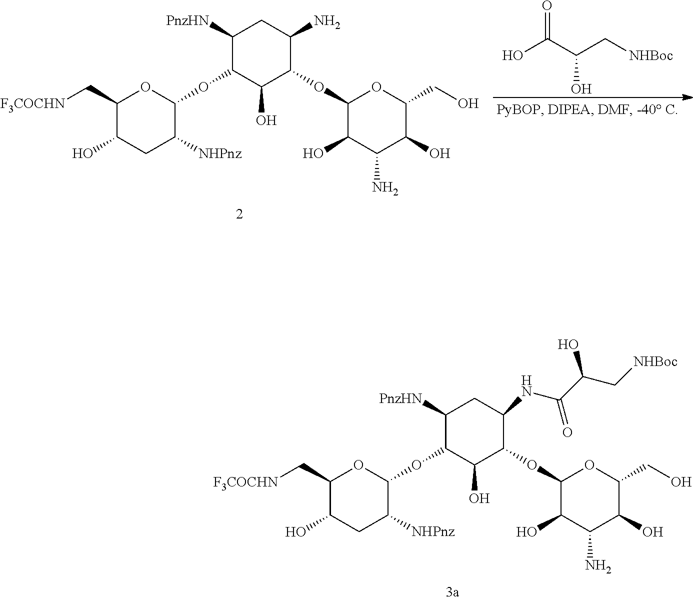

### Method B:

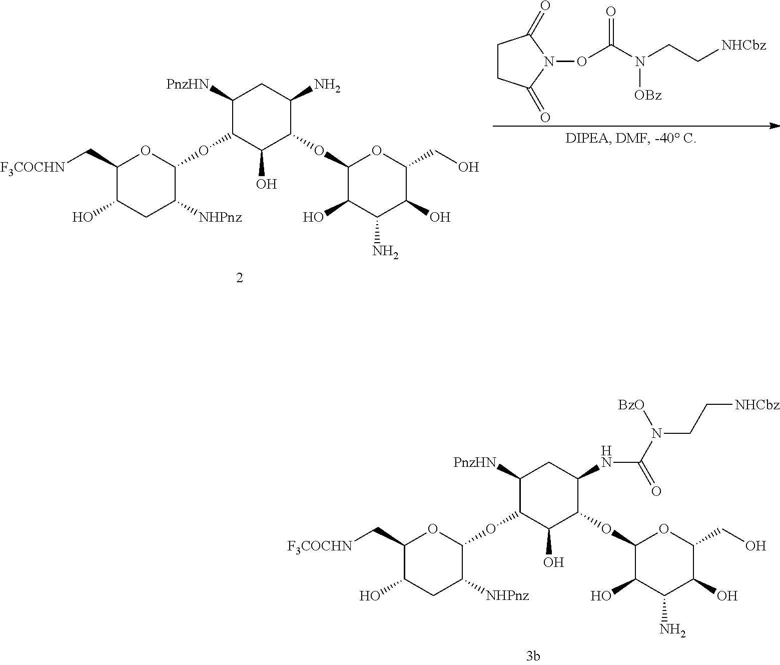

### Example D-2

### N-1 Epoxide Opening

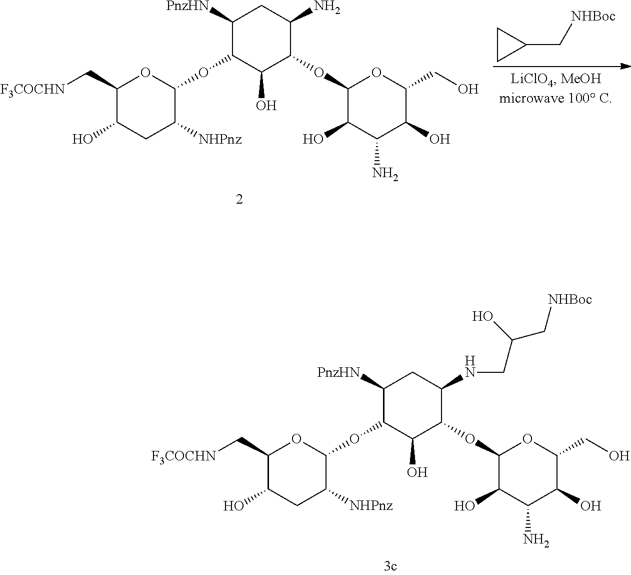

### Example D-3

### N-1 Sulfonylation

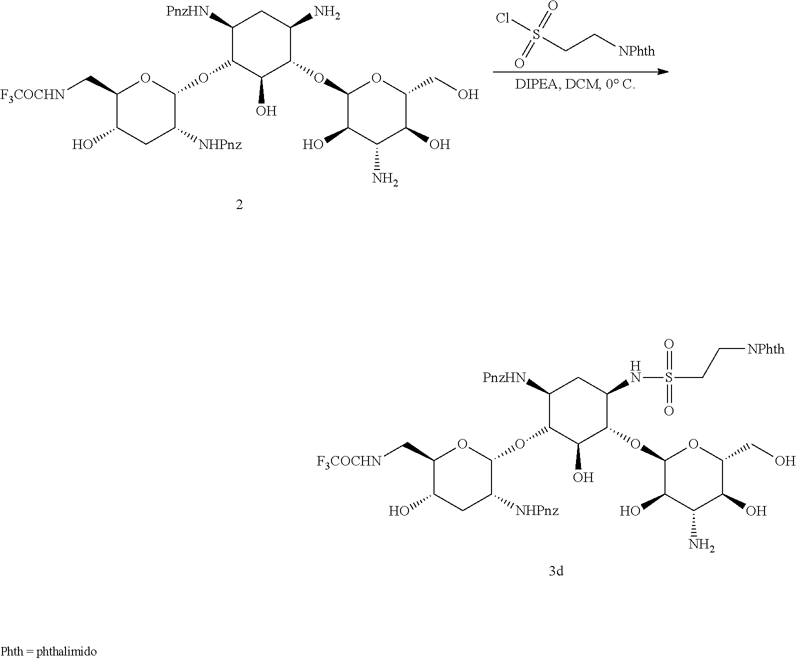

### Example D-4

### N-1 Reductive Amination

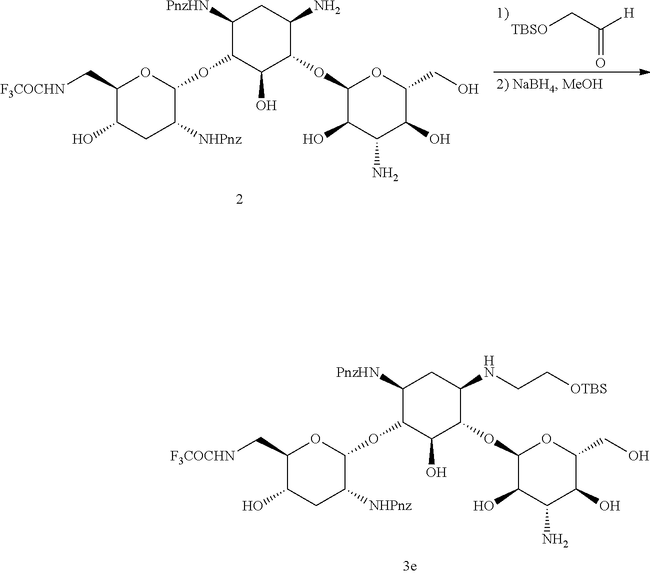

### Example D-5

### N-6′ Reductive Amination

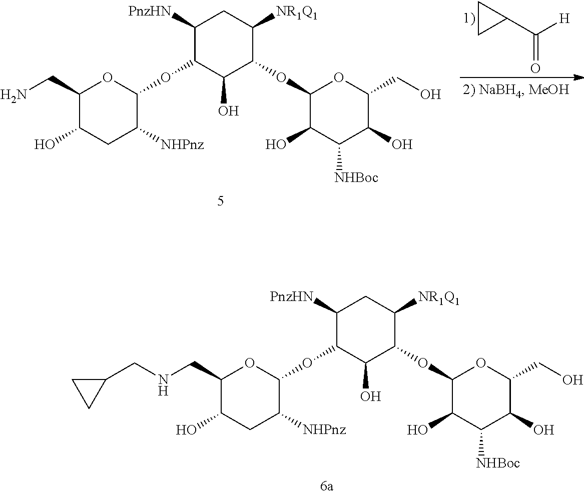

### Example D-6

### N-6′ Epoxide Opening

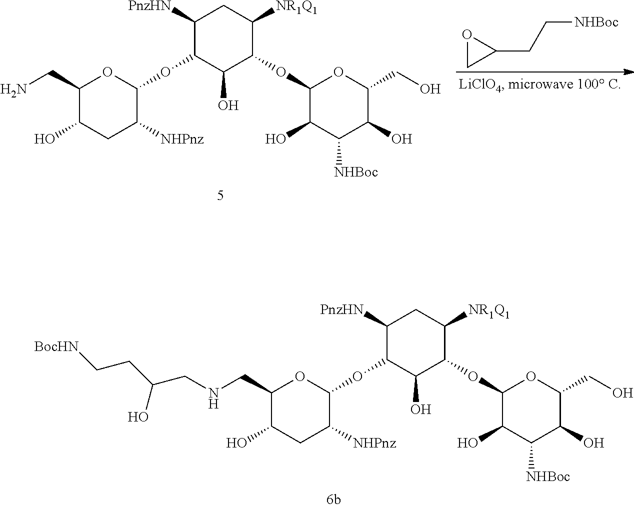

### Example D-7

### N-1 Acylation

### Method A:

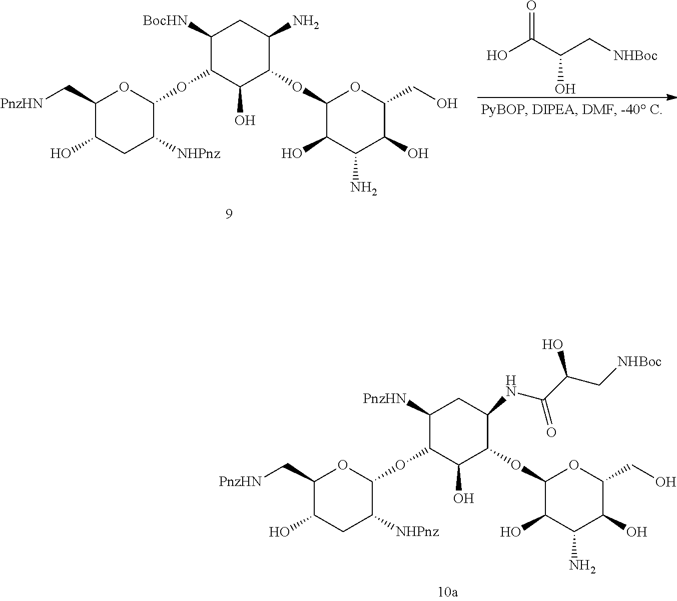

### Method B:

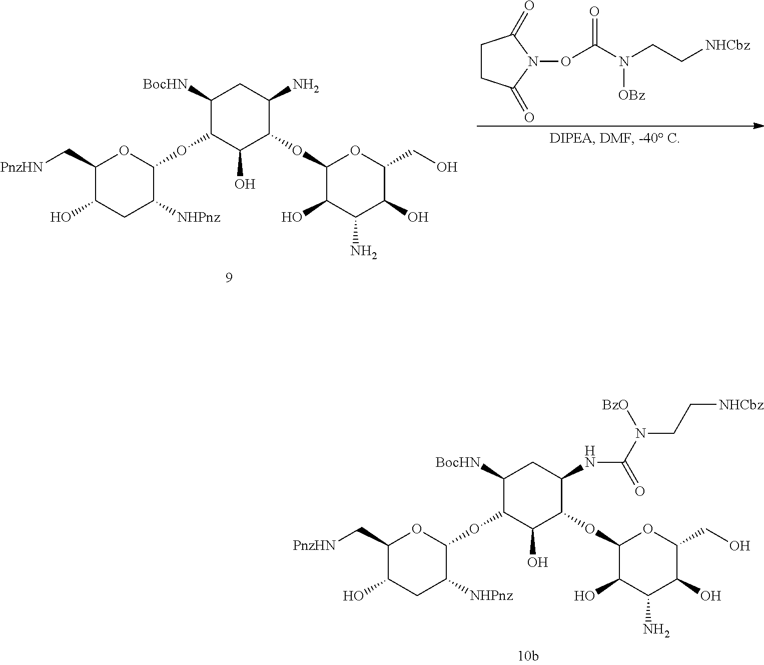

### Example D-8

### N-1 Epoxide Opening

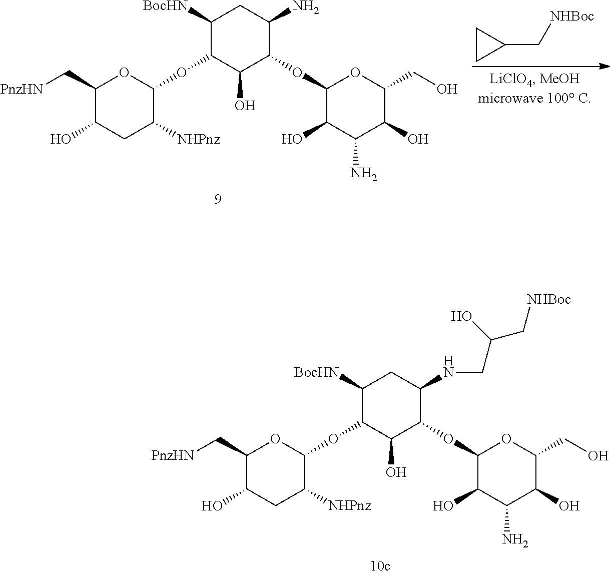

### Example D-9

### N-1 Sulfonylation

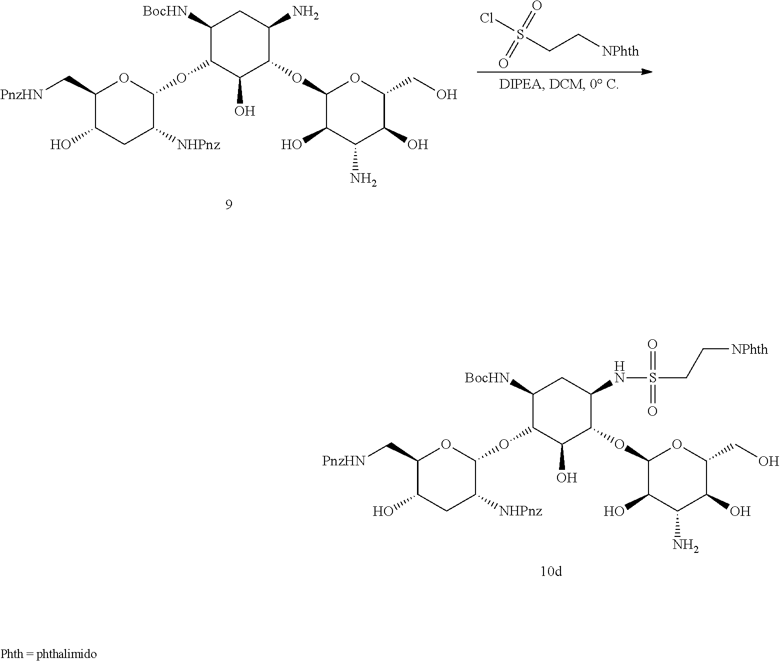

### Example D-10

### N-1 Reductive Amination

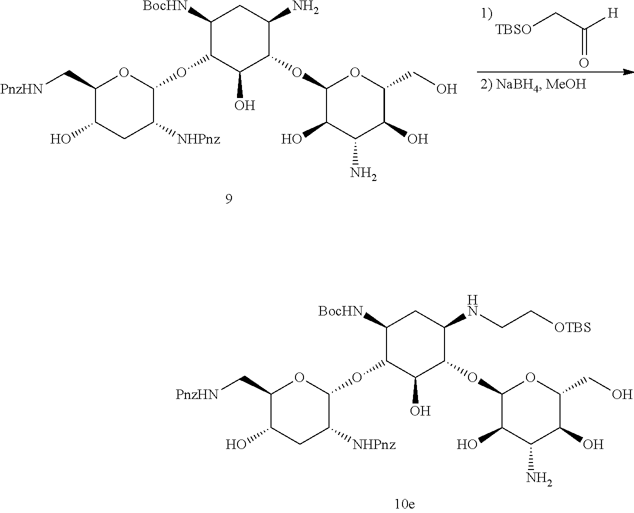

### Example D-11

### N-2′ Reductive Amination

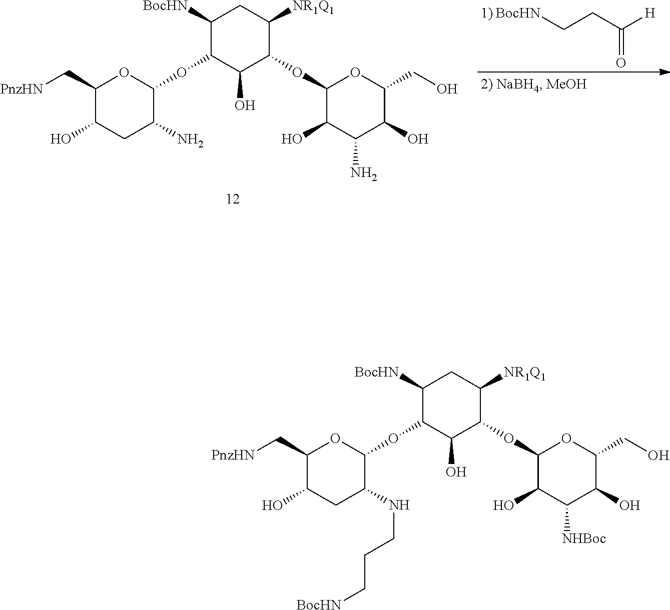

### Example D-12

### N-2′ Epoxide Opening

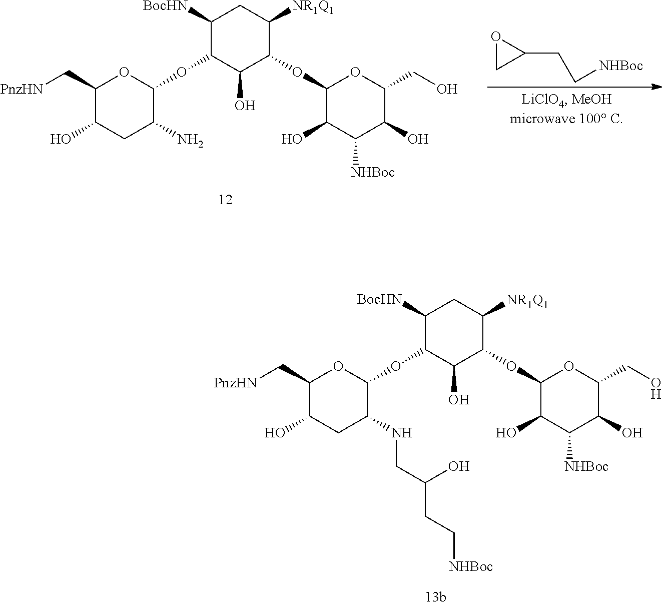

### Example D-13

### N-2′ Guanidinium

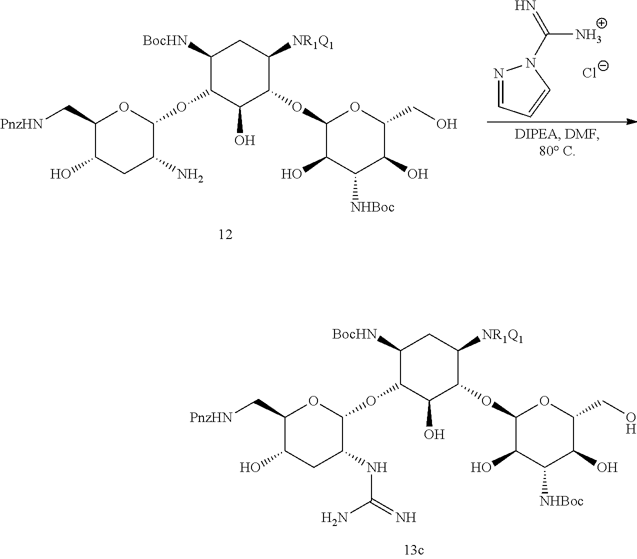

### Example D-14

### N-2′ Acylation

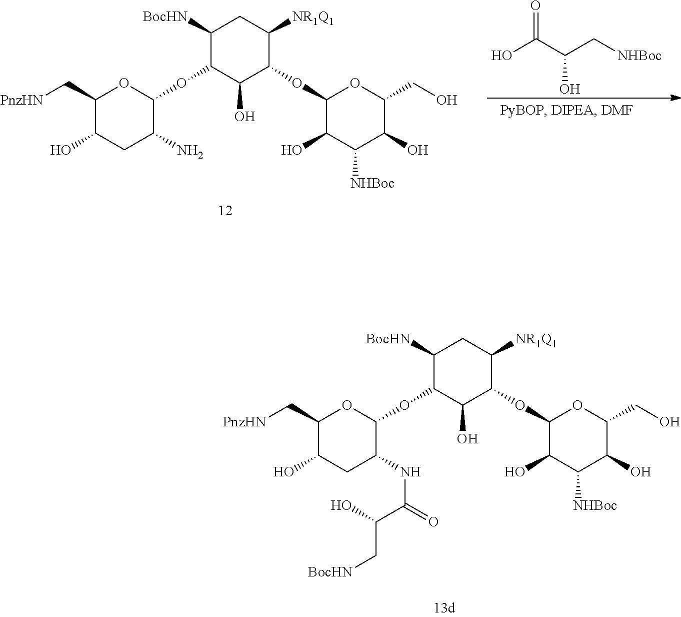

Gentamicin C Analogs

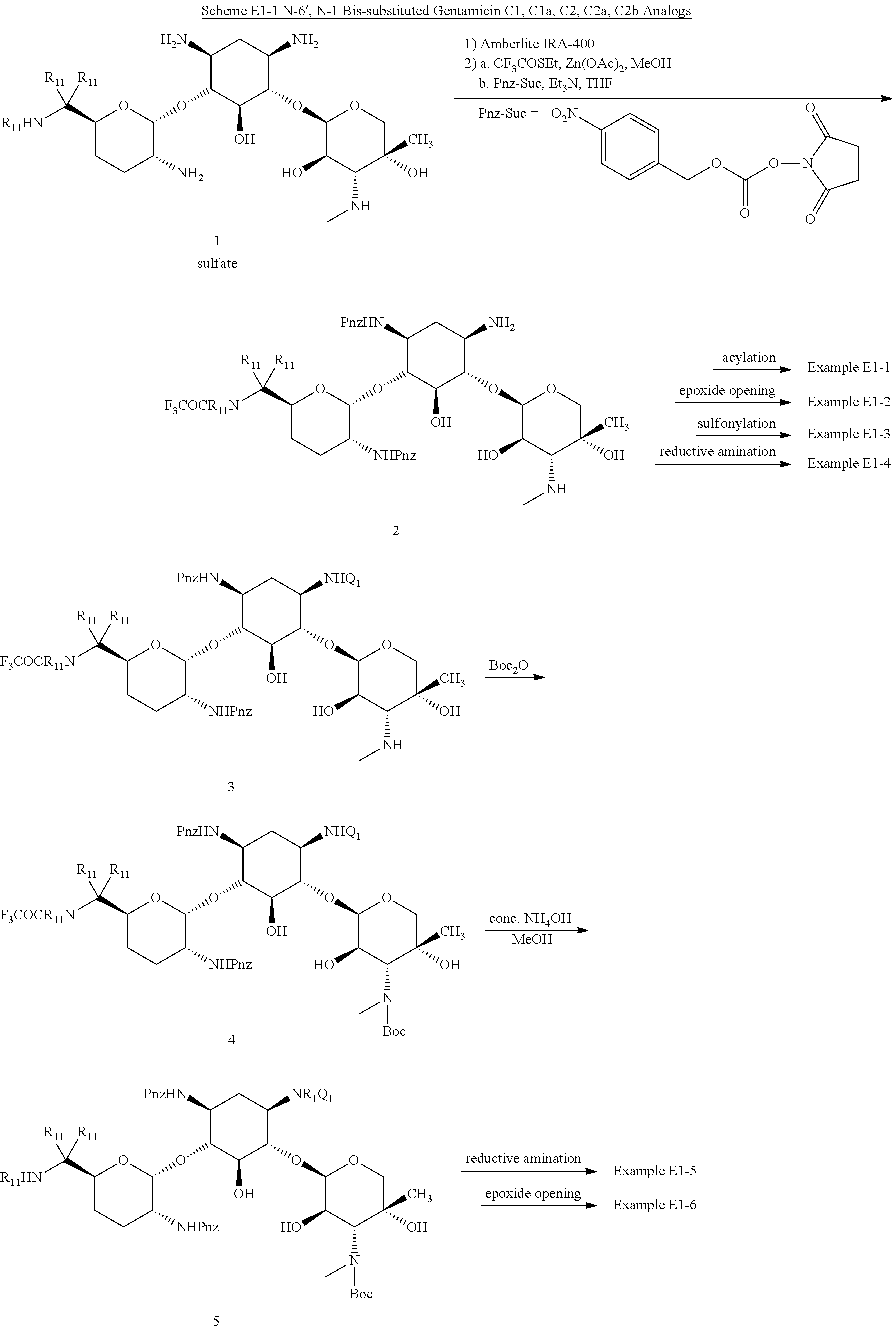

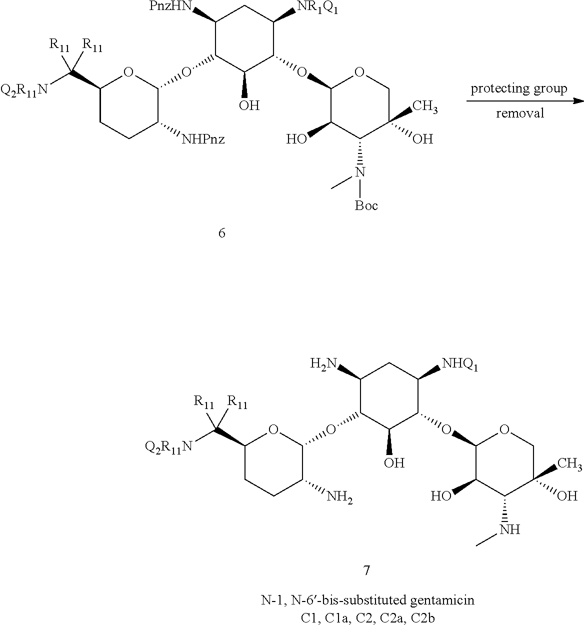

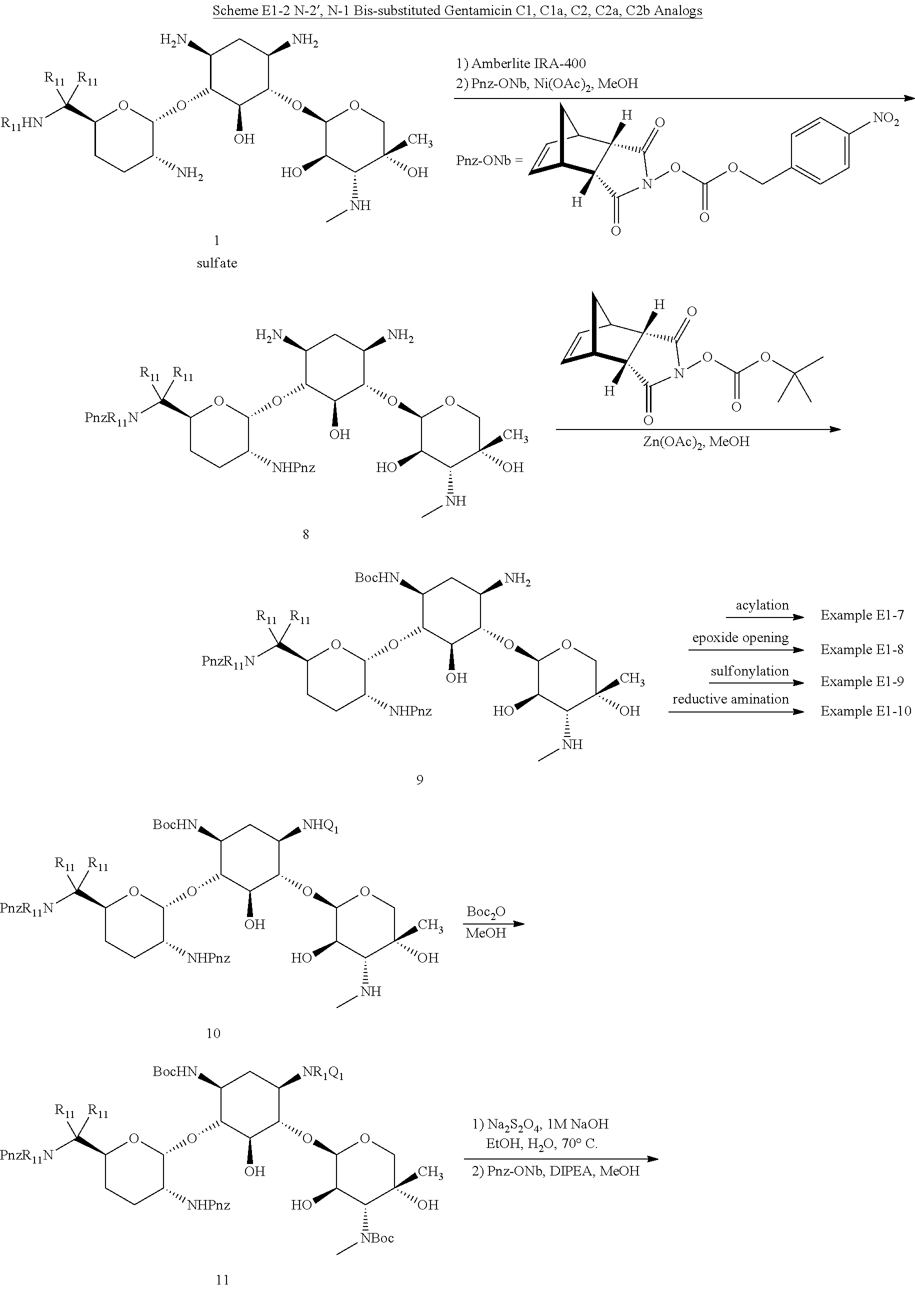

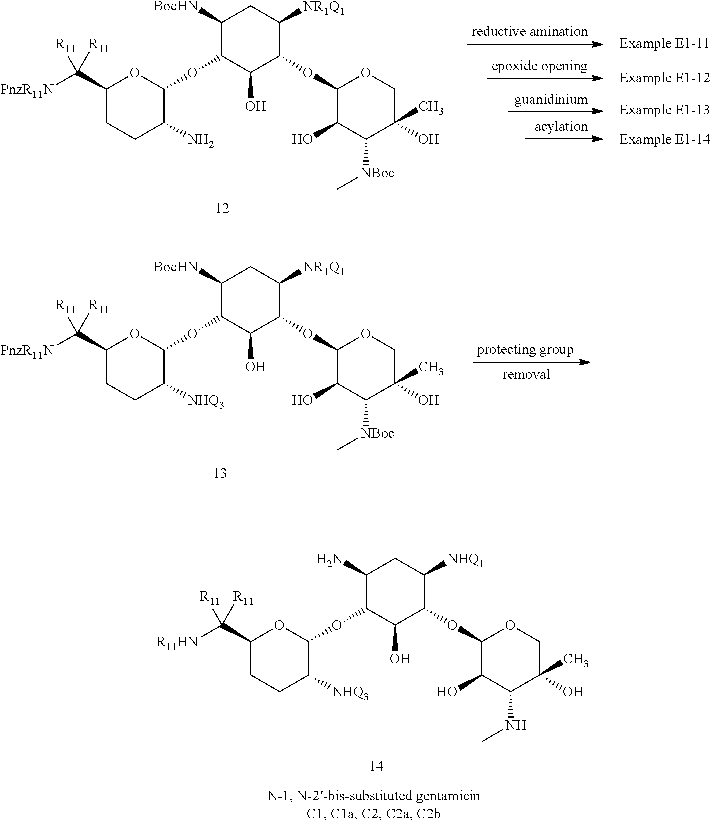

### Example E1-1

### N-1 Acylation

### Method A:

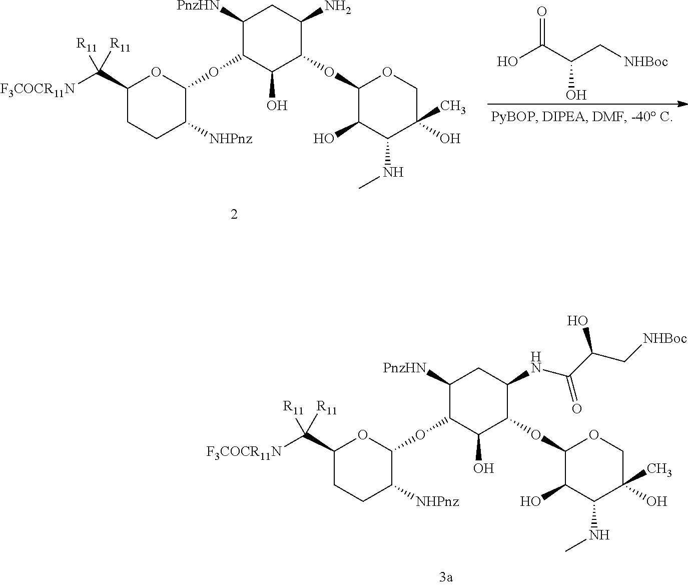

### Method B:

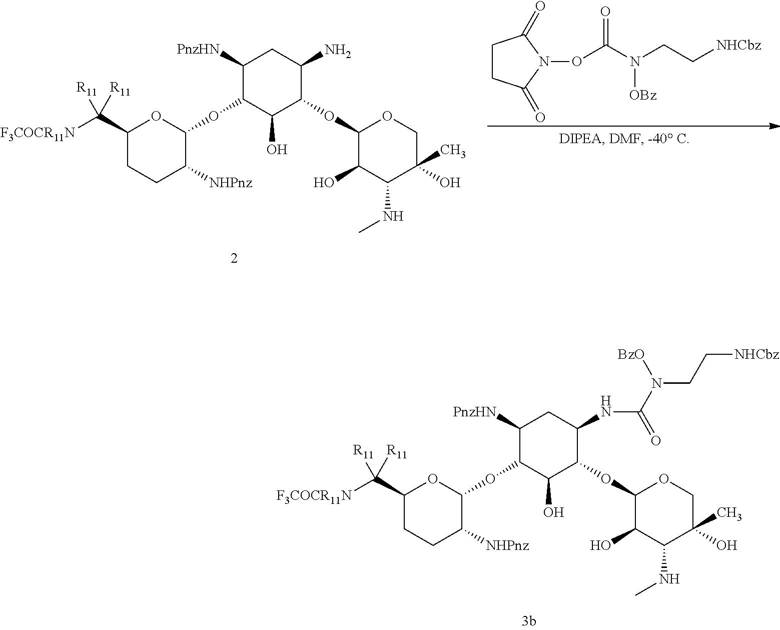

### Example E1-2

### N-1 Epoxide Opening

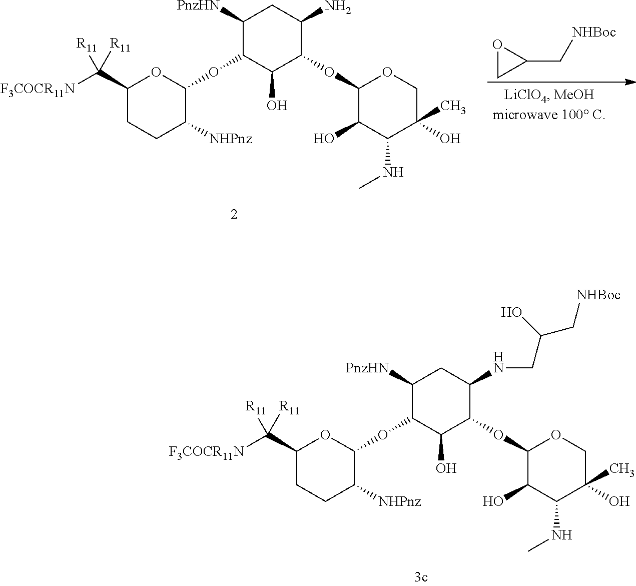

### Example E1-3

### N-1 Sulfonylation

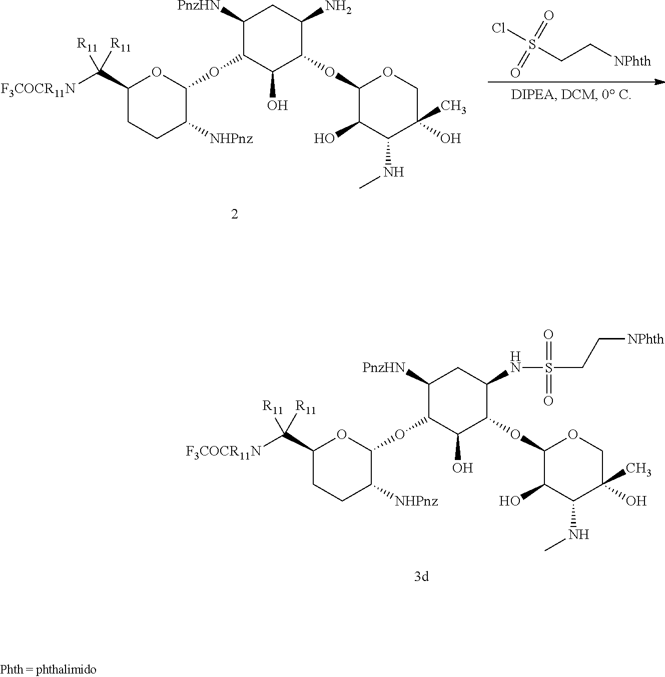

### Example E1-4

### N-1 Reductive Amination

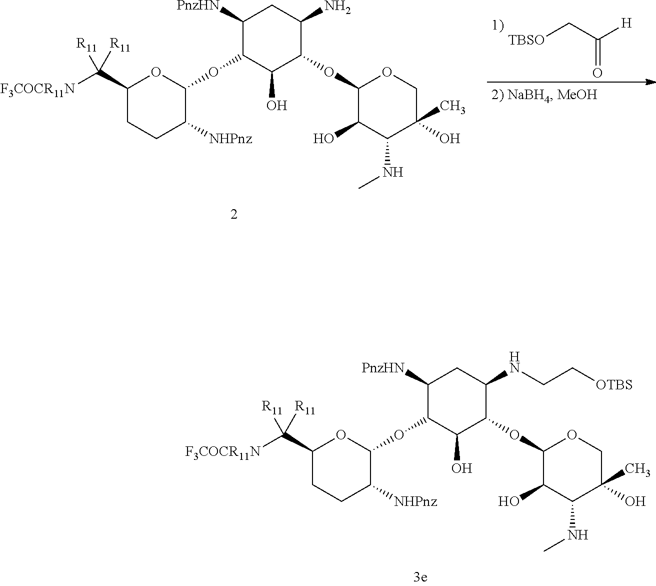

### Example E1-5

### N-6′ Reductive Amination

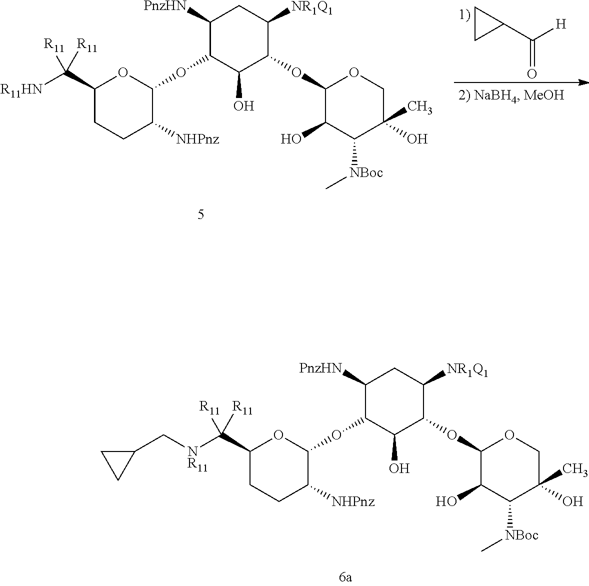

### Example E1-6

### N-6′ Epoxide Opening

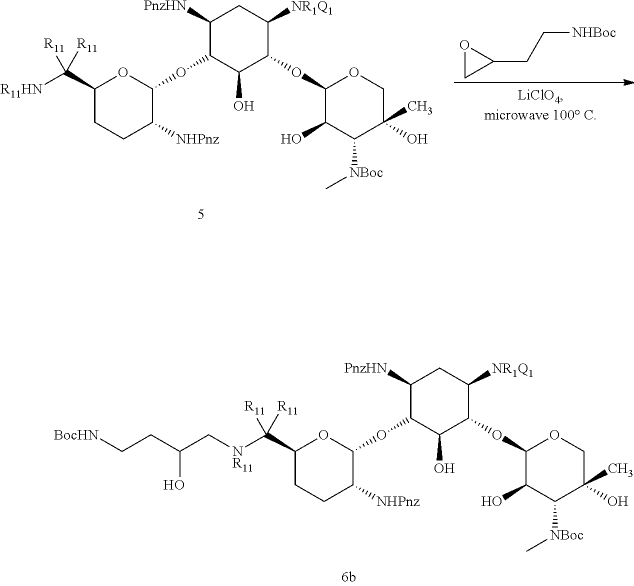

### Example E1-7

### N-1 Acylation

### Method A:

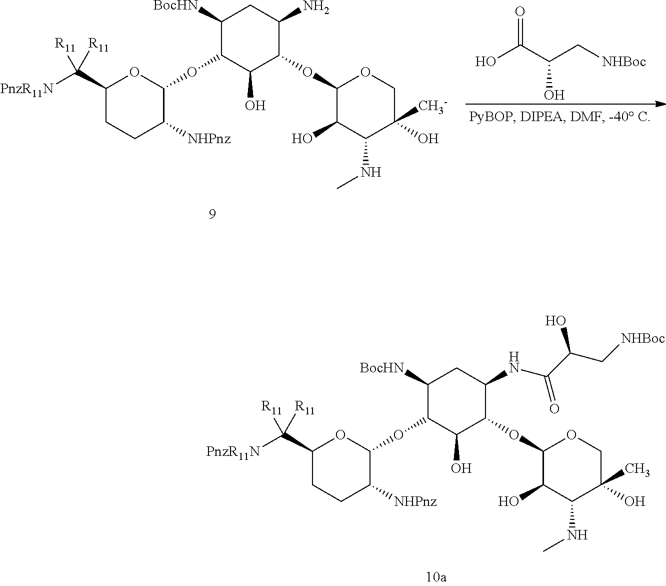

### Method B:

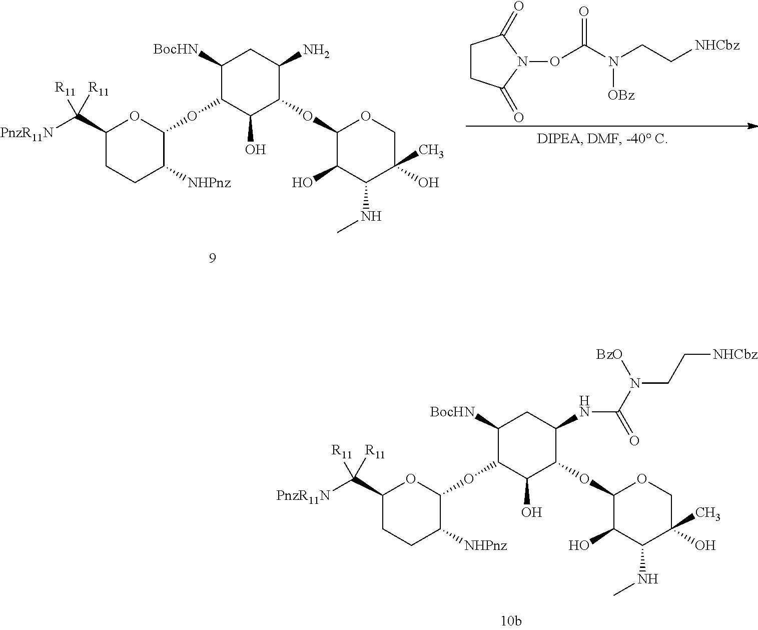

### Example E1-8

### N-1 Epoxide Opening

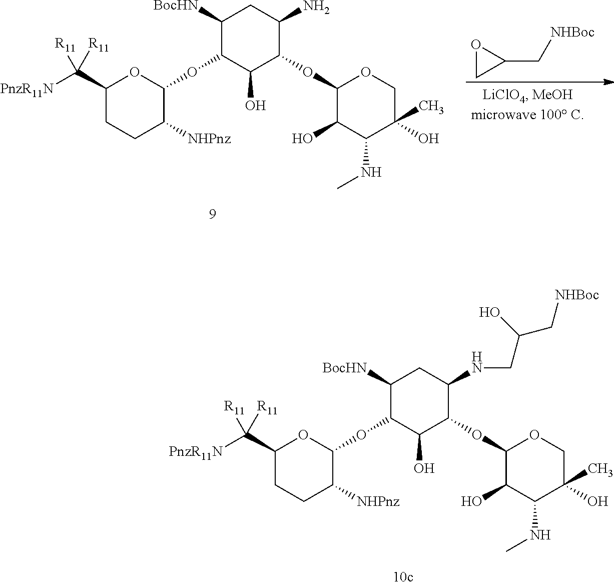

### Example E1-9

### N-1 Sulfonylation

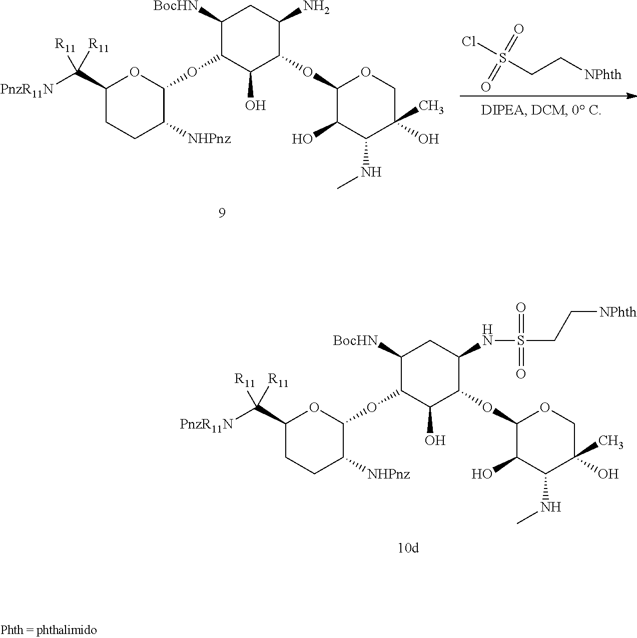

### Example E1-10

### N-1 Reductive Amination

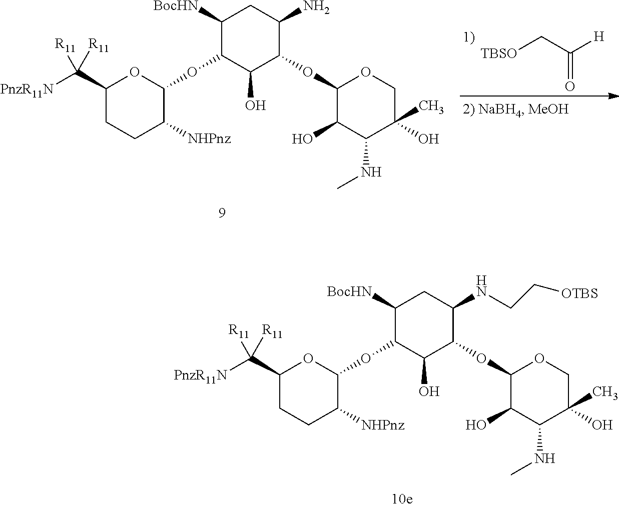

### Example E1-11

### N-2′ Reductive Amination

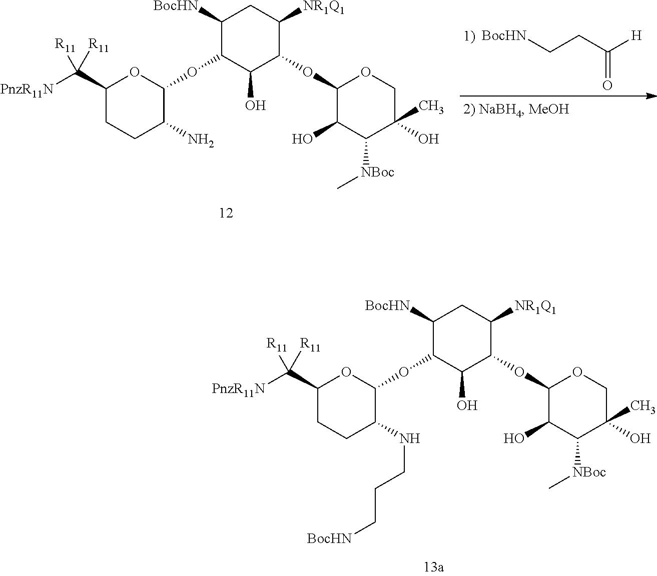

### Example E1-12

### N-2′ Epoxide Opening

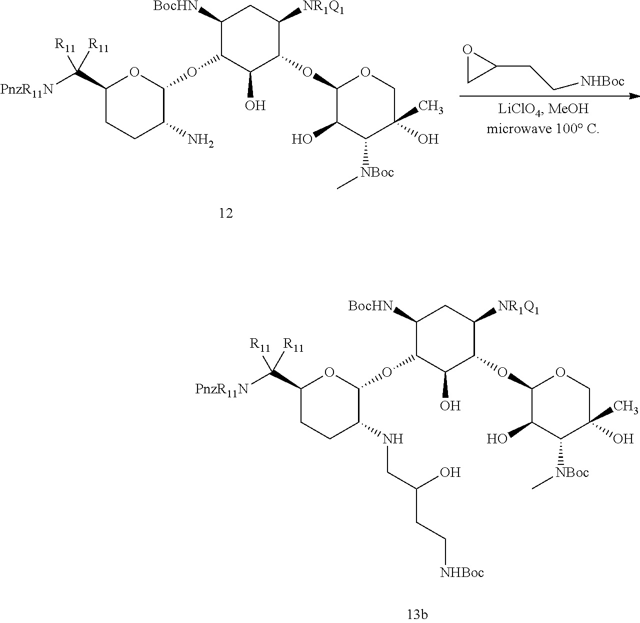

### Example E1-13

### N-2′ Guanidinium

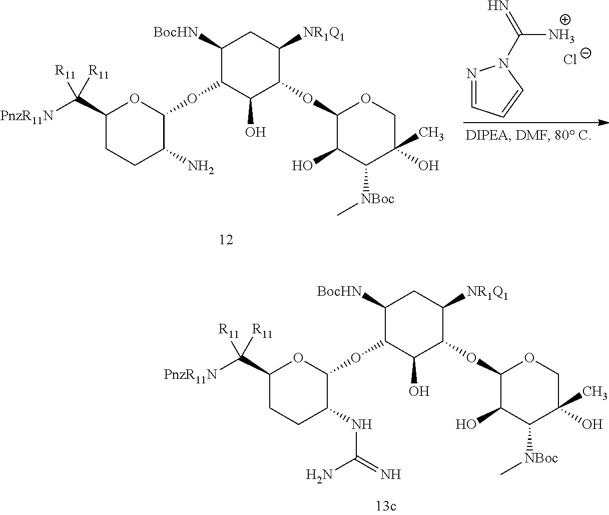

### Example E1-14

### N-2′ Acylation

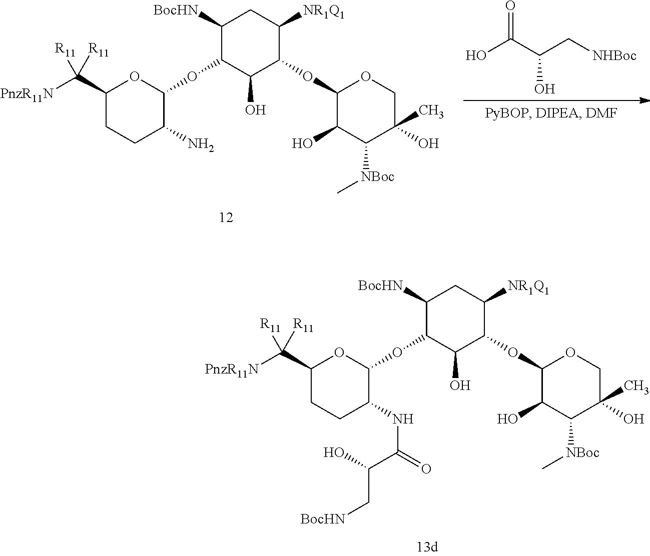

Gentamicin B Analogs

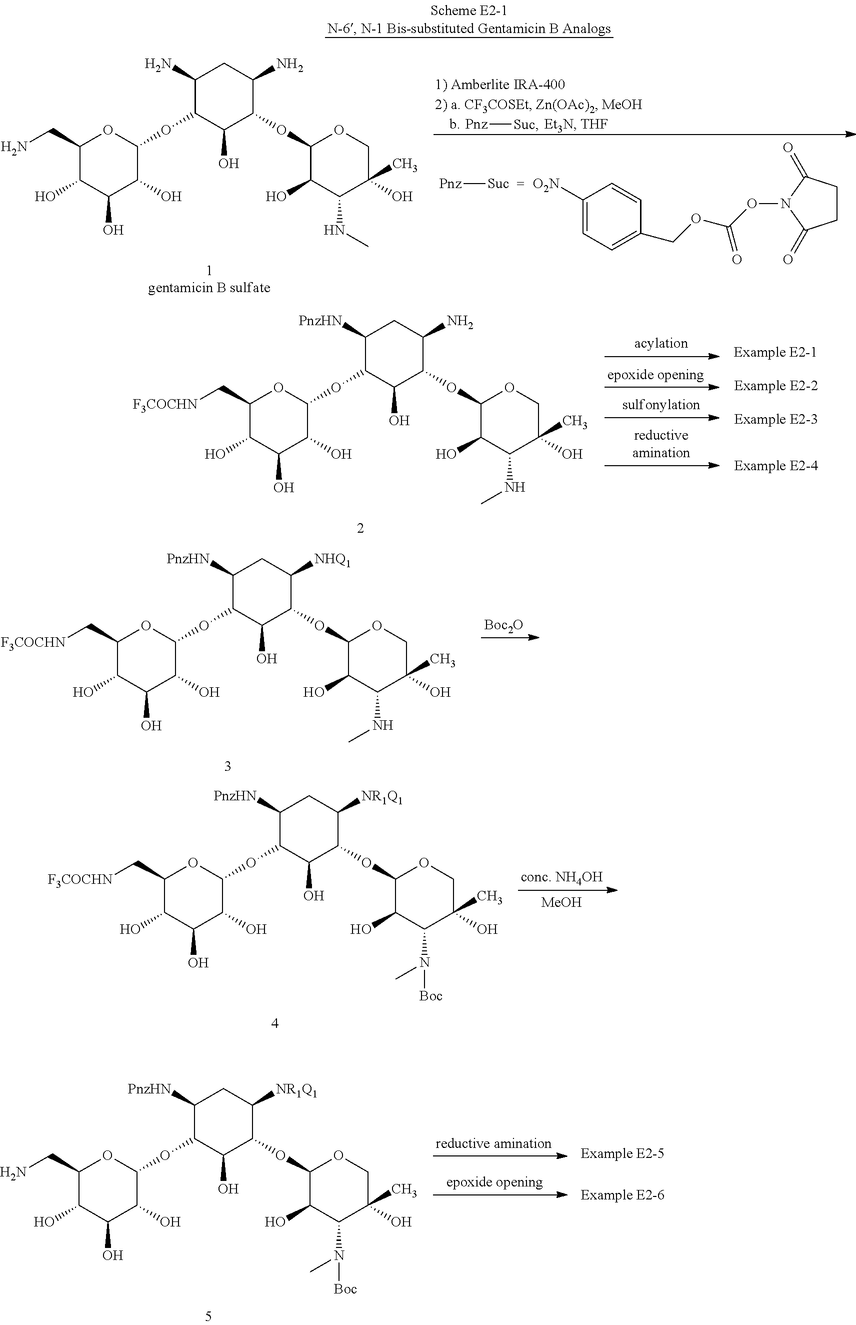

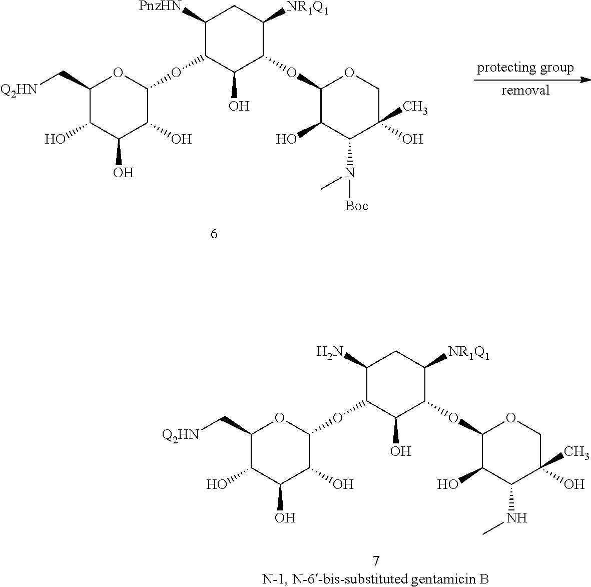

### Example E2-1

### N−1 Acylation

### Method A:

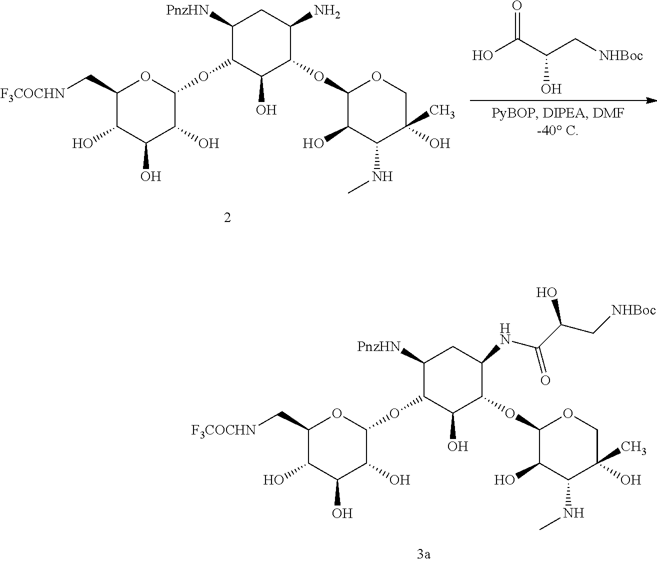

### Method B:

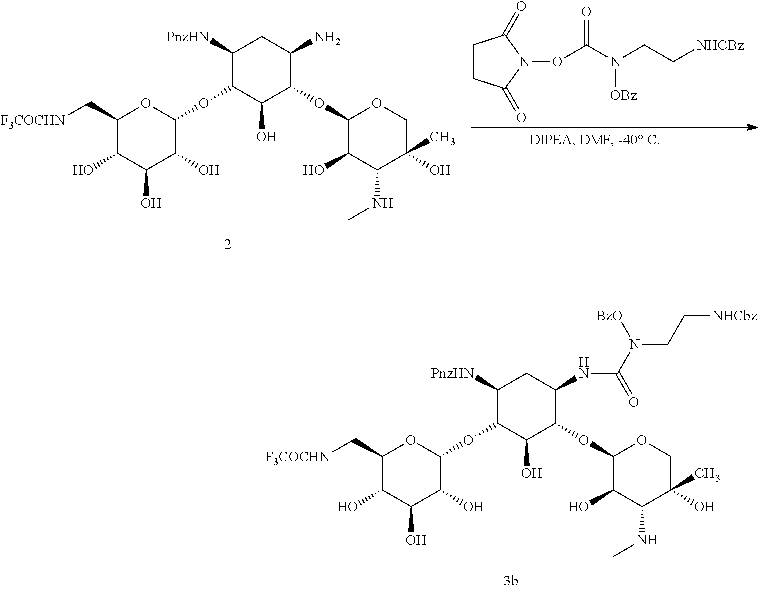

### Example E2-2

### N-1 Epoxide Opening

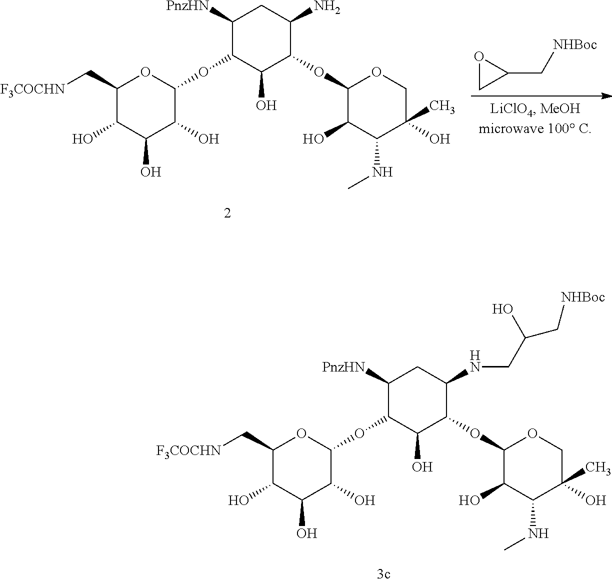

### Example E2-3

### N-1 Sulfonylation

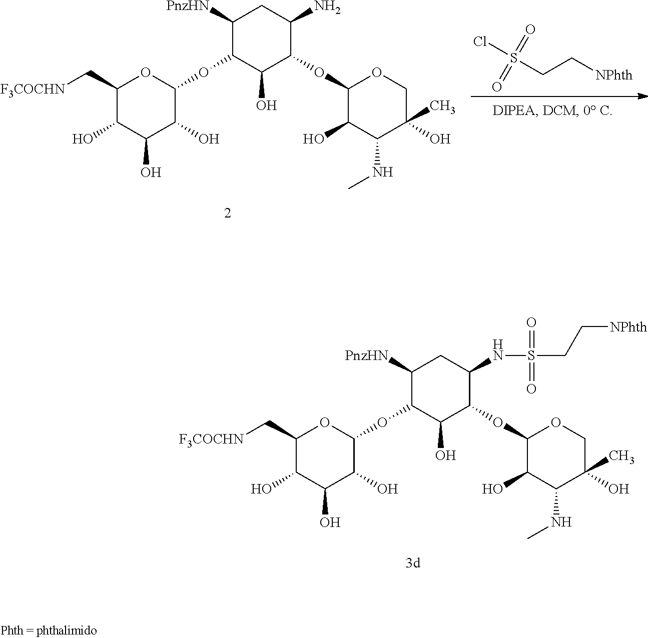

### Example E2-4

### N-1 Reductive Amination

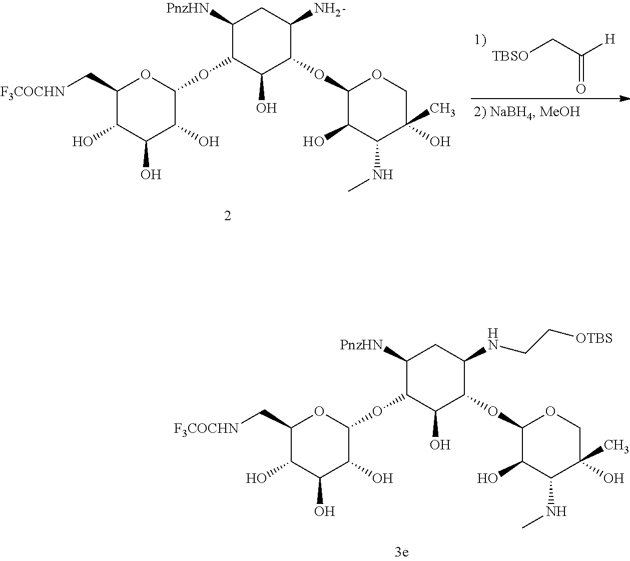

### Example E2-5

### N-6′ Reductive Amination

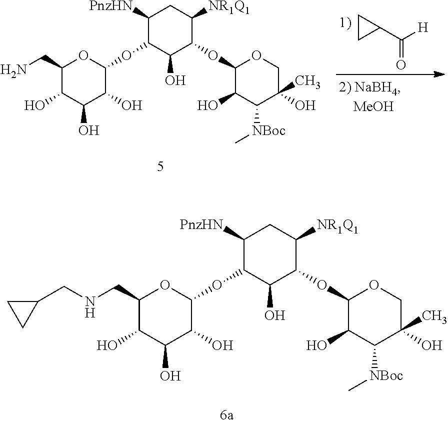

### Example E2-6

### N-6′ Epoxide Opening

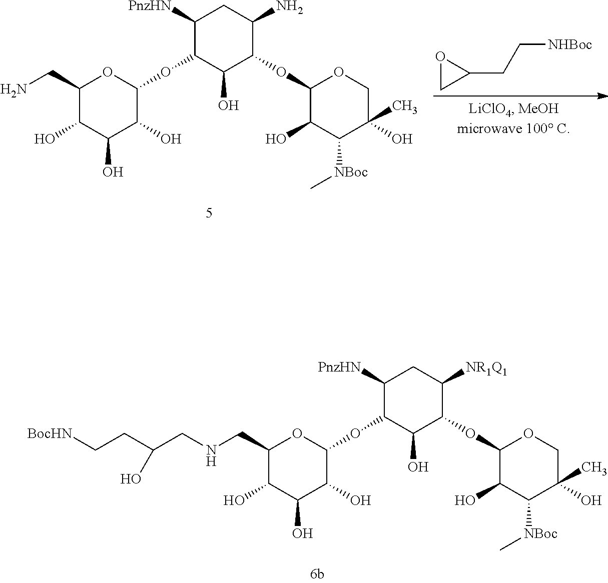

Representative Coupling Agents

As one of skill in the art will appreciate, other representative coupling agents that may be utilized in the above examples include, but are not limited to, the following.

### Representative N-1 Coupling Reagents

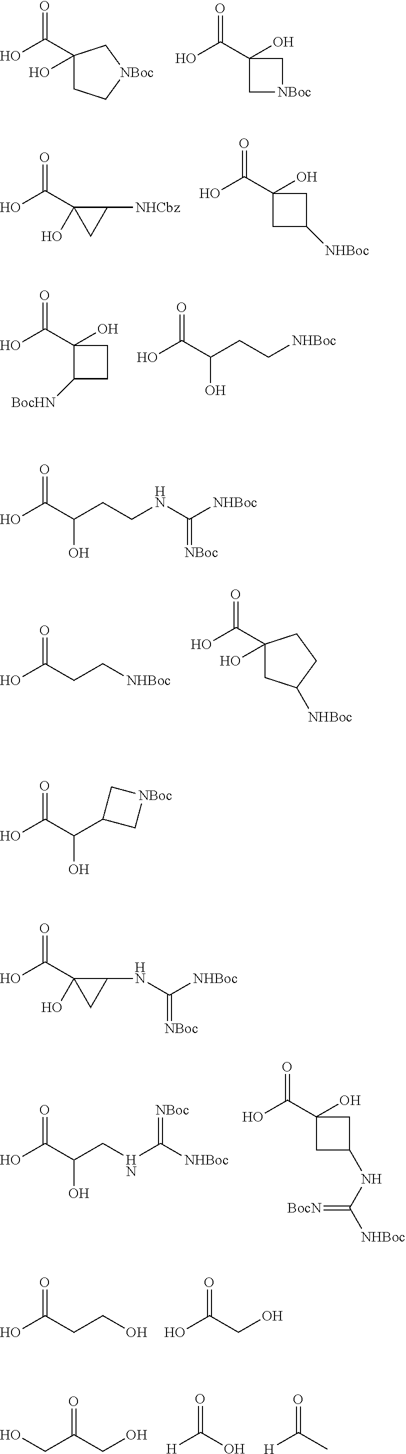

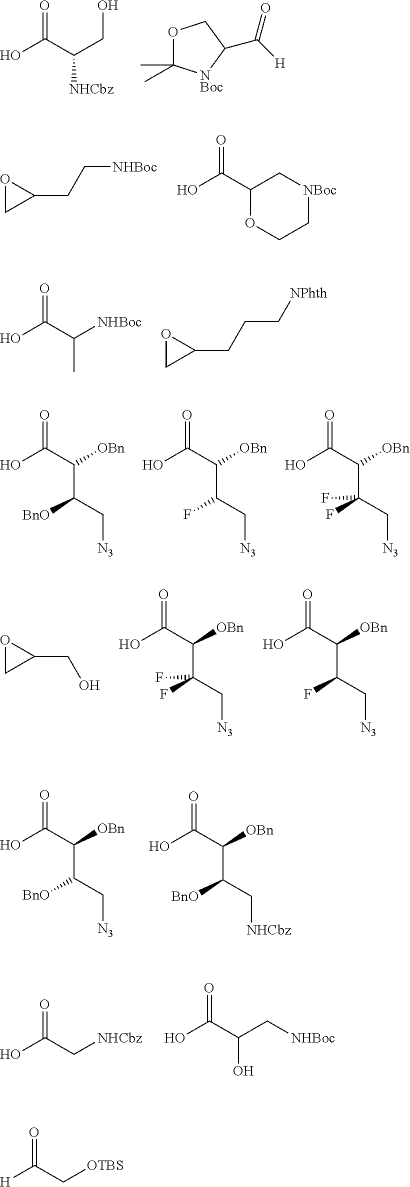

### Representative N-2′ Coupling Reagents

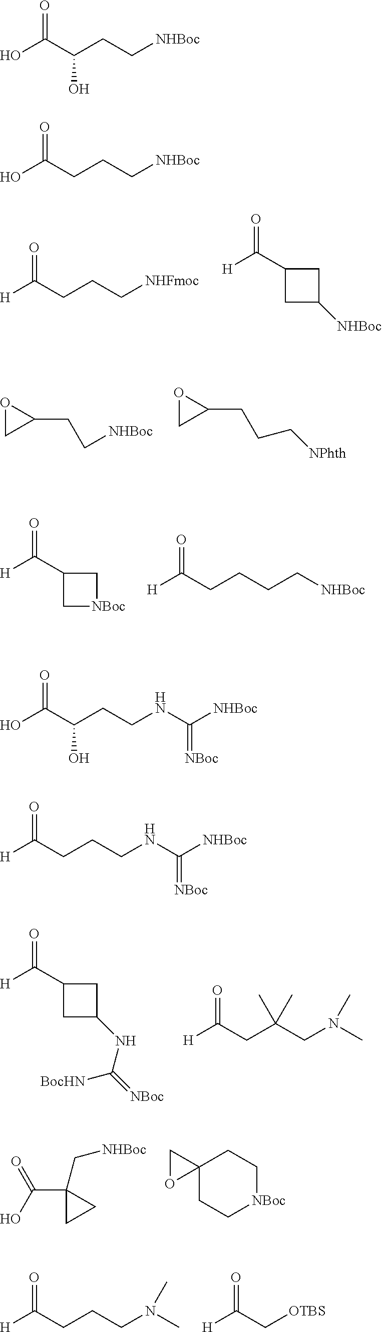

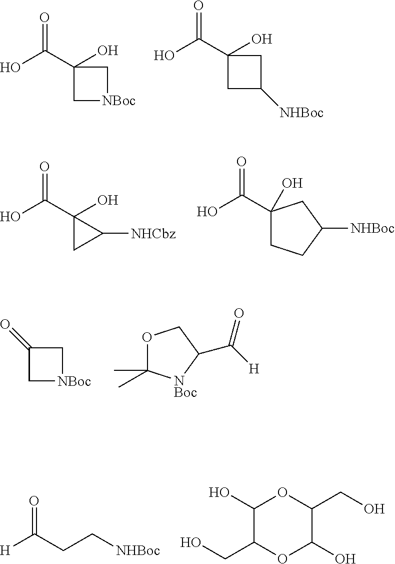

### Representative N-6′ Coupling Reagents

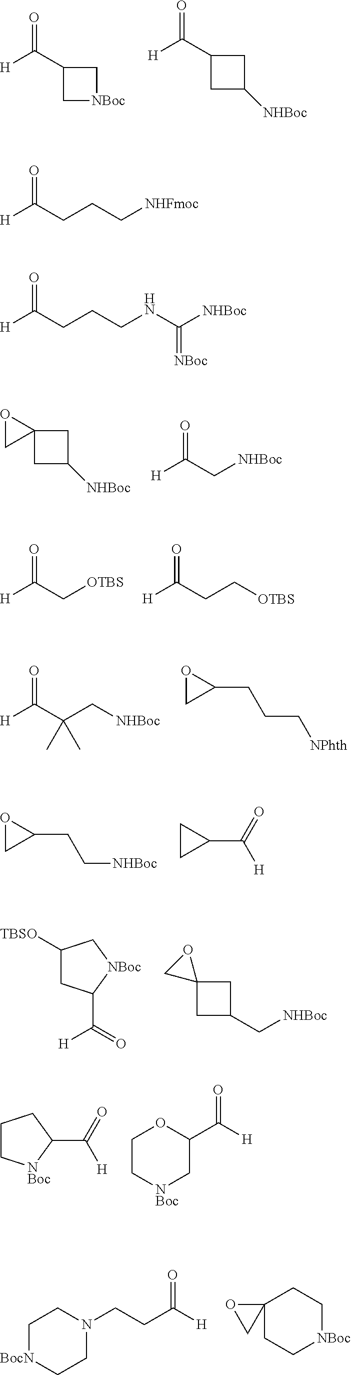

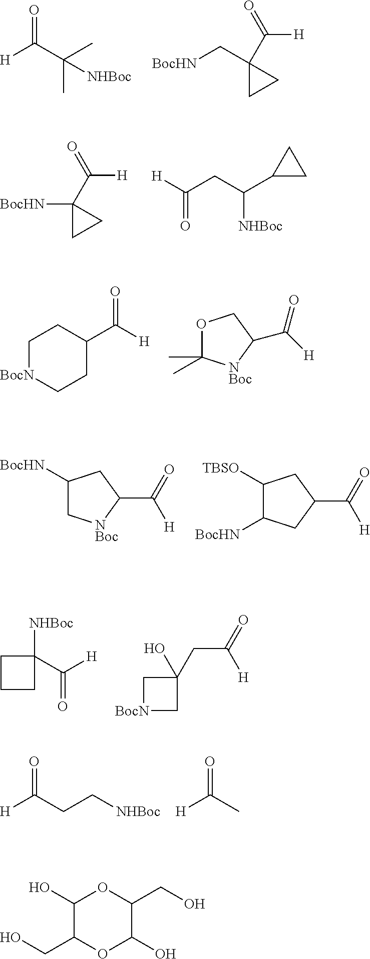

General Synthetic Procedures

### Procedure 1: Reductive Amination

Method A: To a stirring solution of the sisomicin derivative (0.06 mmol) in MeOH (2 mL) was added the aldehyde (0.068 mmol), silica supported cyanoborohydride (0.1 g, 1.0 mmol/g), and the reaction mixture was heated by microwave irradiation to 100° C. (100 watts power) for 15 minutes. The reaction was checked by MS for completeness, and once complete all solvent was removed by rotary evaporation. The resulting residue was dissolved in EtOAc (20 ml), and washed with 5% NaHCO₃ (2×5 mL), followed by brine (5 mL). The organic phase was then dried over Na₂SO₄, filtered and the solvent was removed by rotary evaporation.

Method B: To a solution of sisomicin derivative (0.078 mmol) in DMF (1 ml) were added 3 Å molecular sieves (15-20), followed by the aldehyde (0.15 mmol) and the reaction was shaken for 2.5 hours. The reaction was checked by MS for completeness and, if needed, more aldehyde (0.5 eq) was added. The reaction mixture was then added dropwise to a stirring solution of NaBH₄ (0.78 mmol) in MeOH (2 mL) at 0° C., and the reaction was stirred for 1 hour. The reaction was diluted with H₂O (2 mL) and EtOAc (2 ml). The organic layer was separated and the aqueous layer was extracted with EtOAc (3×3 mL). The combined organic layers were dried over Na₂SO₄, filtered and concentrated to dryness.

Procedure 2: PNZ deprotection

To a stirring solution of the PNZ protected sisomicin derivative (0.054 mmol) in EtOH (1.5 mL) and H₂O (1 mL) was added 1N NaOH (0.3 mL), followed by Na₂S₂O₄ (0.315 mmol), and the reaction mixture was heated at 70° C. for 12 hours. The reaction progress was monitored by MS. Once complete, the reaction mixture was diluted with H₂O (5 mL) and then extracted with EtOAc (2×10 mL). The combined organic layers were washed with H₂O (2×5 mL), brine (5 mL), dried over Na₂SO₄, filtered and concentrated to dryness.

Procedure 3: Boc deprotection (tert-butyl dimethyl silyl protecting group is removed under these conditions)

Important: Before Boc deprotection a sample must be dried well by pumping at high vacuum for 3 h.

Method A: To a stirring solution of the Boc protected sisomicin (0.054 mmol) in DCM (1 mL) were added 3 Å molecular sieves (4-6), and trifluoroacetic acid (0.6 mL). The reaction was stirred at room temperature for 1 h, and checked for completeness by MS. Upon completion the reaction mixture was diluted with ether (15 mL) to induce precipitation. The vial was centrifuged and the supernatant was decanted. The precipitate was washed with ether (2×15 ml), decanted and dried under vacuum.

Method B: To a stirring solution of Boc-protected sisomicin derivative (0.078 mmol) in DCM (1.5 mL) at 0° C. was added trifluoroacetic acid (1.5 mL). The reaction was stirred for 45 minutes, and checked for completeness by MS. Upon completion, the reaction was diluted with dichloroethane (10 ml) and concentrated to dryness. The last dilution/concentration step was repeated twice.

### Procedure 4: BOP and PyBOP Coupling

Method A: To a stirring solution of sisomicin derivative (0.078 mmol) in DMF (1 mL) was added the acid (0.16 mmol), followed by PyBOP (0.16 mmol) and DIPEA (0.31 mmol) and the reaction was stirred overnight. The reaction mixture was diluted with EtOAc (3 mL) and H₂O (3 mL), and the aqueous layer was separated and extracted with EtOAc (3×3 mL). The combined organic layers were dried over Na₂SO₄, filtered and concentrated to dryness.

Method B: To a stirring solution of sisomicin derivative (0.073 mmol) in DMF (1 mL) was added the acid (0.102 mmol), DIPEA (0.43 mmol) and a solution of BOP (0.102 mmol) in DMF (1 mL) and the reaction was stirred for 4 hours, with its progress monitored by MS. The reaction mixture was diluted with water (8 mL) and was extracted with EtOAc (2×10 mL). The combined organic layers were washed with 5% aq. NaHCO₃ (2×3 mL) and brine (3 mL), dried over Na₂SO₄, filtered and concentrated to dryness.

### Procedure 5: Epoxide Opening

To a stirring solution of the sisomicin derivative (0.06 mmol) in MeOH (2 mL) was added the epoxide (0.07 mmol), LiClO₄ (0.15 mmol), and the reaction mixture was heated by microwave irradiation to 100° C. for 90 minutes. The reaction progress was monitored by MS. Upon completion, the solvent was removed by rotary evaporation. The resulting residue was dissolved in EtOAc (20 mL), washed with H₂O (2×5 mL) and brine (5 mL), dried over Na₂SO₄, filtered and concentrated to dryness.

### Procedure 6: Phthalimido Deprotection

To a stirring solution of the phthalimido protected sisomicin (0.064 mmol) in EtOH (3 mL) was added hydrazine (0.32 mmol), and the reaction mixture was heated to reflux for 2 h. The reaction progress was monitored by MS. Upon cooling to room temperature, the cyclic by-product precipitated and was removed by filtration. The filtrate was concentrated to dryness to yield a residue, which was dissolved in EtOAc (20 mL), washed with 5% NaHCO₃ (2×5 mL) and brine (5 mL), dried over Na₂SO₄, filtered and concentrated to dryness.

### Procedure 7: Addition of Guanidinium Group

To a stirring solution of the sisomicin derivative (0.063 mmol) in DMF (1 mL) was added 1H-pyrazole-1-carboxamidine hydrochloride (0.09 mmol), followed by DIPEA (0.862 ml) and the reaction mixture was heated to 80° C. and stirred overnight. The reaction progress was monitored by MS. Upon completion, the reaction mixture was cooled to room temperature and diluted with water (3 mL). The aqueous phase was separated and extracted with EtOAc (2×5 mL), and the combined organics were washed with brine (5 mL), dried over Na₂SO₄, filtered and concentrated to dryness.

### Procedure 8: Nosylation

To a stirring solution of the sisomicin derivative (0.23 mmol) in DCM (20 mL) was added 2-nitrobenzenesulfonyl chloride (0.25 mmol), and DIPEA (0.3 mmol), and the reaction was allowed to stir for 3 h. The reaction progress was monitored by MS. Upon completion, the DCM was removed by rotary evaporation and the resulting residue was dissolved in ethyl acetate (50 mL) and washed with 5% NaHCO₃ (2×10 mL), and brine (10 mL). The combined organic layers were then dried over Na₂SO₄, filtered and concentrated to dryness.

Procedure 9: Nosyl Group deprotection

To a stirring solution of the nosyl protected sisomicin derivative (0.056 mmol) in DMF (1.5 mL) was added benzenethiol (0.224 mmol), K₂CO₃ (1.12 mmol) and the reaction mixture was stirred for 2 hours, with its progress monitored by MS. Upon completion, the reaction mixture was diluted with water (5 mL) and extracted with ethyl acetate (2×10 mL). The combined organic layers were washed with water (2×5 mL) and brine (5 mL), dried over Na₂SO₄, filtered and concentrated to dryness.

### Procedure 10: PNZ Removal by Hydrogenolysis

To a stirring solution of sisomicin derivative (0.41 mmol) in EtOH (60 mL) was added AcOH (0.14 mL), followed by Pd/C (30% by weight). The reaction vessel was evacuated and replenished with H₂ (1 atm), and the reaction mixture was stirred for 6 h. The reaction vessel was then evacuated and replenished with nitrogen. The solids were removed by filtration through a pad of Celite, and washed with MeOH (10 mL). Solvent evaporation gave the desired product.

### Procedure 11: Mono Alkylation

To a stirring solution of the nosyl protected sisomicin derivative (0.072 mmol) in DMF (1.5 mL) was added the halogenated alkane (0.144 mmol), K₂CO₃ (0.216 mmol) and the reaction mixture was heated to 80° C. with its progress monitored by MS. Upon completion, the reaction mixture was diluted with water (2 mL) and extracted with ethyl acetate (2×5 mL). The combined organic layers were washed with brine (1.5 mL), dried over Na₂SO₄, filtered and concentrated to dryness.

### Procedure 12: Sulfonylation

To a stirring solution of the sisomicin scaffold (0.067 mmol) in DCM (3 mL) was added DIPEA (0.128 mol) and the sulfonyl chloride (0.07 mmol). The reaction mixture was stirred at room temperature and its progress was monitored by MS. Once complete, the solvent was removed by rotary evaporation and the residue was dissolved in ethyl acetate (20 mL), washed with 5% NaHCO₃ (2×5 mL) and brine (5 mL), dried over Na₂SO₄, filtered and concentrated to dryness.

### Procedure 13: N-Boc Protection

To a stirring solution of the amine (4.64 mmol) in THF (10 mL) was added 1N NaOH (10 mL), followed by Boc-anhydride (5.57 mmol) and the reaction progress was checked by MS. Once complete, the THF was removed by rotary evaporation and water (40 mL) was added. The aqueous phase was separated and extracted with Et₂O (2×30 ml). The aqueous phase was acidified to pH 3 by the addition of dilute H₃PO₄ and was then extracted with EtOAc (2×60 ml). The combined organic layers were washed with H₂O (2×30 mL) and brine (30 mL), dried over Na₂SO₄, filtered and concentrated to dryness.

### Procedure 14: Syntheses of Epoxides

To a stirring solution of the alkene (5.16 mmol) in chloroform (20 mL) at 0° C. was added m-chloroperbenzoic acid (8.0 mmol) and the reaction mixture was stirred for 30 minutes at 0° C. and was then allowed to warm to room temperature. The reaction progress was monitored by MS and TLC, and additional portions of m-CPBA were added as needed. Upon completion, the reaction mixture was diluted with chloroform (50 mL) and washed with 10% aq. Na₂SO₃ (2×30 mL), 10% aq. NaHCO₃ (2×50 mL) and brine (50 mL). The organic layer was dried over Na₂SO₄, filtered and concentrated to yield a crude product, which was purified by flash choromatography (silica gel/hexanes:ethyl acetate 0-25%).

### Procedure 15: General Procedure for Synthesis of α-Hydroxy Carboxylic Acids

Step #1. O-(Trimethylsilyl) cyanohydrines: A 50-mL flask equipped with a magnetic stirring bar and drying tube was charged with the ketone or aldehyde (0.010 mmol), followed by THF (50 mL), trimethylsilyl cyanide (1.39 g, 14 mmol), and zinc iodide (0.090 g, 0.28 mmol), and the reaction mixture was stirred at room temperature for 24 hr. Solvent evaporation gave a residue, which was dissolved in EtOAc (60 mL), washed with 5% aq. NaHCO₃ (2×30 mL), H₂O (30 mL), and brine (30 mL), dried over Na₂SO₄, filtered and concentrated to dryness to yield a crude, which was carried through to the next step without further purification.

Step #2. Acid hydrolysis to α-hydroxy carboxylic acid: AcOH (25 ml) and conc. HCl (25 ml) were added to the unpurified material from step #1 and the reaction mixture was refluxed for 2-3 hr. The reaction mixture was then concentrated to dryness to give a white solid, which was carried through to the next step without further purification.

Step #3. Boc protection: To a stirring solution of solid from step #2 in 2 M NaOH (20 mL) and i-PrOH (20 mL) at 0° C. was added Boc₂O (6.6 g, 3 mmol) in small portions, and the reaction mixture was allowed to warm to room temperature over 4 h. i-PrOH was then evaporated, and H₂O (50 mL) was added, and the aqueous phase was separated and extracted with Et₂O (2×30 ml). The aqueous layer was acidified to pH 3 by addition of dilute H₃PO₄ and was extracted with EtOAc (2×60 ml). The combined organic layers were washed with H₂O (2×30 mL) and brine (30 mL), dried over Na₂SO₄, filtered and concentrated to yield the desired N-Boc-α-hydroxy carboxylic acids in 56-72% yield.

Aldehydes and ketones used: N-Boc-3-Pyrrolidonone, N-Boc-3-azetidinone, N-Boc-4-piperidone and N-Boc-3-azetidincarboxaldehyde.

### Procedure 16: Protection of Amine by Fmoc Group

To a stirring solution of the amine (0.049 mol) in DCM (100 mL), was added DIPEA (16 mL, 0.099 mol) and the reaction mixture was cooled to 0° C. Fmoc-Cl (12.8 g, 0.049 mol) was then added portion-wise over several minutes, and the reaction was allowed to warm to room temperature for 2 hr. The organic layer was washed with water (2×50 mL) and brine (50 mL), dried over Na₂SO₄, filtered and concentrated to dryness to yield the Fmoc protected amine (90-95% yield).

### Procedure 17: Mitsunobu Alkylation

To a stirring solution of the nosylated sisomicin derivative (0.087 mmol) in toluene (2.5 mL) was added the alcohol (0.174 mmol), triphenylphosphine (0.174 mmol) and the reaction mixture was cooled in a 4° C. refrigerator for 10 minutes. A cooled solution of DEAD (0.174 mmol in 2 mL anhydrous toluene) was then added and the reaction was allowed to shake overnight. The reaction progress was monitored by MS, and additional alcohol and triphenylphosphine were added if needed. Once complete, ethyl acetate (30 mL) was added and the organic phase was washed with 5% aq. NaHCO₃ (2×5 mL) and brine (5 mL), dried over Na₂SO₄, filtered and concentrated to dryness.

### Procedure 18: Synthesis of Aldehydes via TEMPO/Bleach Oxidation

To a vigorously stirring solution of the alcohol (1.54 mmol) in DCM (4 mL) was added TEMPO (0.007 g, 0.045 mmol, 0.03 mol %) and a 2M aqueous KBr solution (75 mL, 0.15 mmol, 10 mol %) and the reaction mixture was cooled to −10° C. In a separate flask NaHCO₃ (0.5 g, 9.5 mmol) was dissolved in bleach (25 mL, Chlorox 6.0% NaOCl) to yield a 0.78 M buffered NaOCl solution. This freshly prepared 0.78 M NaOCl solution (2.3 mL, 1.8 mmol, 117 mol %) was added to the reaction mixture over min and the reaction was stirred for an additional 30 min at 0° C. The organic phase was separated and the aqueous layer was extracted with dichloromethane (2×4 mL). The combined organic layers were washed with 10% aq. Na₂S₂O₃ (4 mL), sat. aq. NaHCO₃ (2×4 mL), brine (5 mL), dried over Na₂SO₄ and concentrated to dryness.

Procedure 19: Synthesis of alcohols via Borane Reduction

To a stirring solution of the acid (1.5 mmol) in THF (5 mL) at −10° C. was slowly added 1.0M BH₃-THF (2.98 mL, 2.98 mmol). The reaction mixture was stirred vigorously for an additional 3 min at −10° C., and was then allowed to warm to room temperature overnight. The reaction was quenched by the dropwise addition of a solution of HOAc/H₂O (1:1 v/v, 2.0 mL). The THF was removed by rotary evaporation and sat. aq. NaHCO₃ (15 mL) was added. The aqueous layer was extracted with DCM (3×5 mL) and the combined organic layers were washed with sat. aq. NaHCO₃ (2×5 mL), brine (10 mL), dried over Na₂SO₄, filtered and concentrated to dryness.

Procedure 20: EDC coupling

To a stirring solution of sisomicin derivative (0.048 mmol) in DMF (0.3 mL) and THF (0.6 mL) was added EDC (0.058 mmol), followed by HONb (0.062 mmol), and the acid (0.058 mmol) and the reaction was allowed to stir overnight. The reaction was quenched with H₂O (2 mL) and EtOAc (4 mL) was added. The organic layer was washed with sat. aq. NaHCO₃, sat. aq. NH₄Cl, dried over Na₂SO₄, filtered and concentrated to dryness.

### Procedure 21: Ozonolysis and Pinnick Oxidation

The substrate olefin (0.5 to 0.75 mmol) was dissolved in DCM (30 mL) and the reaction was cooled to −78° C. Ozone was bubbled through until a blue color persisted (3 to 5 min), and the reaction was stirred for 1 hr. Argon was then bubbled through to remove excess ozone for 10 minutes. The reaction was further quenched by the addition of dimethyl sulfide (10 equiv.), and was stirred for 30 min with warming to rt. The solvent was reduced under vacuum to yield the crude aldehyde, which was dried under high-vacuum for 10 min, and used without further purification. To a stirring solution of the aldehyde in THF, tBuOH and H₂O (3:3:2, 10 mL), was added NaH₂ PO₄ (4 equiv.) followed by 2-methyl-2-butene (10 equiv.) and sodium chlorite (2 equiv.), and the reaction was stirred for 4 hr. The reaction mixture was then added to sat. aq. NaCl (10 mL) and extracted with DCM (3×). The combined organic layers were dried over Na₂SO₄, filtered and reduced under vacuum to yield a crude, which was purified by flash chromatography (silica gel, 0→0.5 or 1% MeOH/DCM).

### Procedure 22: Hydrogenolysis

To a stirring solution of aminoglycoside (0.031 mmol) in AcOH (2 mL), was added H₂O (1 mL), followed by Pd(OH)₂/C (40 mg) and the reaction was stirred under a hydrogen atmosphere for 3 hours. The catalyst was removed by filtration, and the reaction was diluted with water and lyophilized to yield a crude, which was purified on a 1-inch reverse phase HPLC column (buffered with 10 mM NH₄₀H) to yield the desired product.

General Purification Procedures

### Method #1: Purification by Basic Condition

### Mobile Phases:

A—Water with 10 mM NH₄OH

B—Acetonitrile with 10 mM NH₄OH

### Columns:

A: Waters-XTerra Prep MS C18 OBD Column
19×100 mm, 5 μm
Gradient: 20 min at 0%, then 0-20% in 200 min at a flow of 20 ml/min

B: Waters-XTerra Prep MS C18 OBD Column
50×100 mm, 5 μm
Gradient: 20 min at 0%, then 0-20% in 200 min at a flow of 20 ml/min

Using the Waters-XTerra, collection was triggered by MS signal. Collected fractions were dried by lyophilization and analyzed by LC/MS/ELSD. Pure fractions were combined and analyzed by LC/MS/ELSD for final purity check. Quantitation was done by LC/MS/CLND system.

### Method #2: Purification by Acidic Condition

### Mobile Phases:

A—Water with 0.1% TFA

B—Acetonitrile with 0.1% TFA

### Columns:

A: Microsorb BDS Dynamax
21.4×250 mm, 10 μm, 100 Å
Gradient: 0-100%, flow 25 ml/min

B: Microsorb BDS Dynamax
41.4×250 mm, 10 μm, 100 Å
Gradient: 0-100%, flow 45 ml/min

### Method #3: Hydrophilic Interaction Chromatography (HILIC) Purification

### Buffers:

Buffer A −3400 ml of Acetonitrile
−600 ml of Water
−15 ml of Acetic Acid
−15 ml of TEA

Buffer B −4000 ml of Water
−100 ml of TEA
−100 ml of Acetic Acid

### Column: PolyC-PolyHydroxyethyl A

150×21 mm, 5 um

Gradient: 20-70% 10 ml/35 min

ELSD signal was used to trigger the collection. Fractions were dried by lyophilization and analyzed by LC/MS/ELSD. Pure fractions were then combined, diluted with water, and lyophilized. Dried fractions were again dissolved in water and lyophilized for a third time to ensure complete removal of TEA. Any samples showing traces of TEA went through additional drying. For delivery, purified compounds were dissolved in >10 mg/ml concentration. Final purity check was done by LC/MS/ELSD and quantitation by LC/MS/CLND.

Representative Intermediates

### Aminoglycoside Freebasing

Amberlite IRA-400 (OH form) (200 g) was washed with MeOH (3×200 ml). To a stirring suspension of the washed resin in MeOH (150 mL) was added aminoglycoside sulfate (20 g) and the mixture was stirred overnight. The resin was then filtered and washed with MeOH (100 mL) and the combined organic layers were concentrated to dryness to yield the desired aminoglycoside.

### Sisomicin

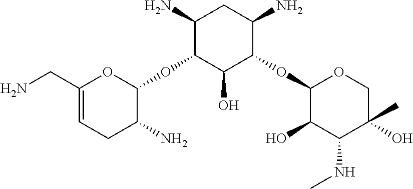

Amberlite IRA-400 (OH form) (200 g) was washed with MeOH (3×200 ml). To a stirring suspension of the washed resin in MeOH (150 mL) was added sisomicin sulfate (20.0 g, 0.029 mol) and the mixture was stirred overnight. The resin was then filtered and washed with MeOH (100 mL) and the combined organic layers were concentrated to dryness to yield the desired sisomicin (11.57 g, 0.026 mol, 89.6% yield): MS m/e [M+H]⁺ Calcd 448.3. Found 448.1.

### (N-Hydroxy-5-norbornene-2,3-dicarboxyl-imido)-4-nitro-benzoate

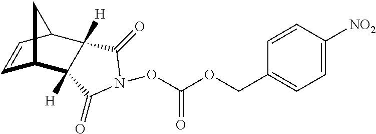

To a stirring solution of 4-nitrobenzyl chloroformate (5.0 g, 0.023 mol) in THF (90 mL) at 0° C. was added N-hydroxy-5-norbornene-2,3-dicarboximide (4.16 g, 0.023 mol), followed by the dropwise addition of a solution of Et₃N (3.2 mL, 0.02 mol) in THF (50 mL) and the reaction was stirred for 4 hours with gradual warming to room temperature. The reaction vessel was then placed in the freezer (−5° C.) for 1 hour to induce precipitation of triethylamine hydrochloride, which was removed by filtration. The filtrate was concentrated to dryness to yield a residue, which was vigorously stirred in MeOH (80 mL) for 1 h and then filtered to yield (N-hydroxy-5-norbornene-2,3-dicarboxyl-imido)-4-nitro-benzoate as a white solid (7.98 g, 0.022 mol, 96% yield): TLC (hexanes:EtOAc v/v 1:1) Rf=0.35.

### 2,5-Dioxo-pyrrolidin-1-yl-4-nitrobenzyl carbonate (PNZ-succinimide)

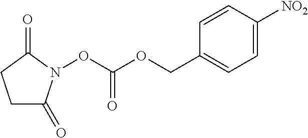

To a stirring solution of N-hydroxysuccinimide (5.35 g, 46.5 mmol) in anhydrous THF (100 mL) was added para-nitrobenzylchloroformate (10.0 g, 46.5 mmol), and the solution was cooled in an ice bath. Triethylamine (6.5 mL, 4.89 g, 46.5 mmol) was added over 10 minutes, and, after 30 minutes, the reaction mixture was allowed to warm to room temperature and stir overnight. The slurry was cooled in an ice-bath, and was filtered, followed by rinsing with ethyl acetate. The filtrate was concentrated in vacuo, and the residue was triturated with methanol. The solids were isolated by filtration to give 2,5-dioxopyrrolidin-1-yl-4-nitrobenzyl carbonate.

### 6′-Trifluoroacetyl-2′,3-diPNZ-sisomicin

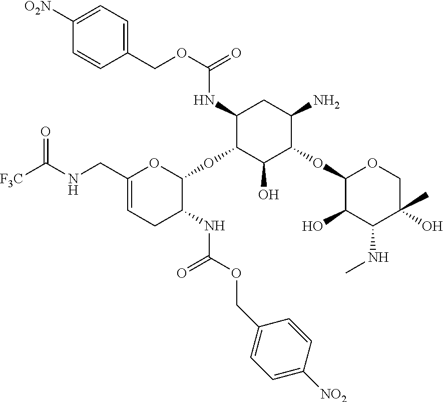

To a stirring solution of sisomicin (30.1 g, 0.067 mol) in MeOH (700 mL) was added zinc acetate (37.07 g, 0.202 mol), followed by the slow addition of a solution of S-ethyltrifluorothioacetate (9.37 mL, 0.074 mol) in MeOH (100 mL) and the reaction was allowed to stir under N₂ overnight. A solution of triethylamine (37.5 mL, 0.27 mol) and PNZ-succinimide (64.2 g, 0.179 mol) in THF (1 L) was then added dropwise, and the reaction was stirred for 3 hours. Solvent evaporation gave a crude, which was dissolved in DCM (2 L) and washed with conc. NH₄OH:H₂O (3:1 v/v, 2×800 mL) and brine (800 mL), dried over MgSO₄, filtered and concentrated to dryness. The residue was dissolved in ethyl acetate (1 L) and extracted with AcOH: H₂O ( 1/9 v/v 1 L). The aqueous layer was washed with ethyl acetate (2×1 L), basified to pH 12 with 10N NaOH, and extracted with ethyl acetate (2×1 L). The organic layer was washed with brine (500 mL), dried over MgSO₄, filtered and concentrated to yield a residue. The crude was dissolved in ethyl acetate (500 mL), and the solution was allowed to stand overnight. The precipitated solids were removed by filtration and the remaining filtrate was concentrated to give a crude, which was purified by RP HPLC Method 2-Column B to yield the desired 6′-trifluoroacetyl-2′,3-diPNZ-sisomicin (MS m/e [M+H]⁺ Calcd 902.3. Found 902.2.

### 6′-Trifluoroacetyl-2′,3-diPNZ-1-acetyl-3″-Boc-sisomicin

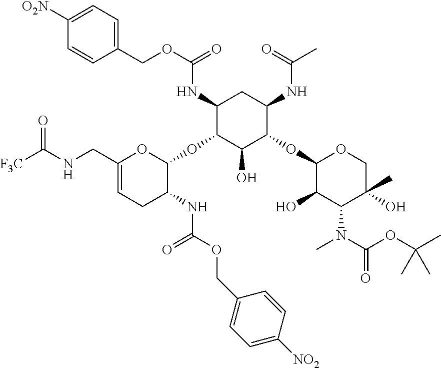

To a stirring solution of 6′-trifluoroacetyl-2′,3-diPNZ-sisomicin (0.7 g, 0.77 mmol) in MeOH (7 mL) at 0° C. was slowly added acetic anhydride (0.095 mL, 1.01 mmol) and the reaction was allowed to warm to room temperature overnight. The reaction was followed by MS, which confirmed the complete formation of the intermediate 6′-trifluoroacetyl-2′,3-diPNZ-1-acetyl-sisomicin (MS m/e [M+H]⁺ Calcd 944.3. Found 944.2, [M+Na]⁺ 966.3). The reaction mixture was then cooled to 0° C. and DIPEA (0.54 mL, 3.11 mmol) was added, followed by Boc anhydride (0.53 mL, 2.33 mmol) and the reaction was stirred for 6 hours with its progress followed by MS. The reaction was quenched with glycine (0.29 g, 3.88 mmol) and K₂CO₃ (0.54 g, 3.88 mmol), and the reaction was stirred overnight. After solvent evaporation, the residue was partitioned between H₂O (10 mL) and EtOAc (10 ml). The aqueous layer was separated and further extracted with EtOAc (3×10 mL), and the combined organic layers were dried over Na₂SO₄, filtered and concentrated to dryness to yield the desired 6′-trifluoroacetyl-2′,3-diPNZ-1-acetyl-3″-Boc-sisomicin (MS m/e [M+H]⁺ Calcd 1044.4. Found 1044.0, [M+Na]⁺ 1066.3), which was carried through to the next step without further purification.

### 2′,3-diPNZ-1-acetyl-3″-Boc-sisomicin

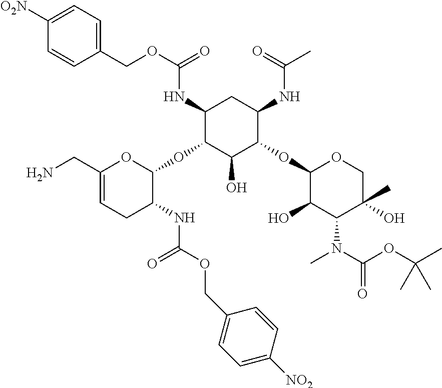

To a stirring solution of 6′-trifluoroacetyl-2′,3-diPNZ-1-acetyl-3″-Boc-sisomicin (0.77 mmol) in MeOH (5 mL) was added conc. NH₄OH (8.2 mL) and the reaction was stirred overnight. Solvent evaporation gave a crude, which was purified by RP HPLC Method 2-Column B to yield the desired 2′,3-diPNZ-1-acetyl-3″-Boc-sisomicin (0.35 g, 0.36 mmol, 46.7% yield, >95% purity): MS m/e [M+H]⁺ Calcd 948.4. Found 948.2.

### N-PNZ-4-amino-2(S)-hydroxy-butyric acid

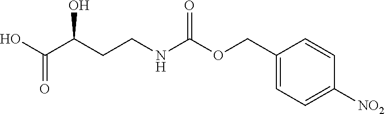

To a stirring solution of 4-amino-2(S)-hydroxybutyric acid (5.0 g, 0.041 mol) in dioxane: H₂O (200 mL, 1:1 v/v) was added K₂CO₃ (11.6 g, 0.084 mol), followed by p-nitrobenzyl chloroformate (9.23 g, 0.043 mol) and the reaction mixture was stirred overnight. The resulting precipitate was removed by filtration and the organic solvent was removed by rotary evaporation. The resulting aqueous solution was acidified to pH 1 by the addition of 1M HCl (100 mL). Upon the addition of ethyl acetate (100 mL) to the aqueous layer, the product precipitated and was collected by filtration. The filtrate was added to a separatory funnel and the organic layer was separated. Upon addition of ethyl acetate (100 mL) to the aqueous layer, a second precipitation occurred, the product was collected by filtration and this process was repeated once more. The combined organic layers were then placed at −5° C. overnight, to induce precipitation of the product, which was collected by filtration. The desired N—PNZ-4-amino-2(S)-hydroxy-butyric acid (9.3 g, 0.031 mol, 75% yield, 90% purity) was carried through to the next step without further purification. MS m/e [M+H]⁺ Calcd 299.1. Found 298.9.

### (N-Hydroxy-5-norbornene-2,3-dicarboxyl-imido)-N-PNZ-4-amino-2(S)-hydroxy-butanoate

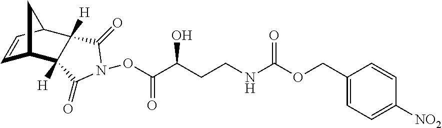

To a stirring solution of N-PNZ-4-amino-2(S)-hydroxy-butyric acid (8.95 g, 30.0 mmol) in THF (200 mL) at 0° C. was slowly added DCC (6.8 g, 33.0 mmol) and the reaction was stirred for 30 min. A solution of N-hydroxy-5-norbornene-2,3-dicarboxylic acid imide (6.45 g, 36.0 mmol) in THF (100 mL) was then added dropwise over 1 hour. The precipitated urea was removed by filtration and the remaining filtrate was concentrated to dryness. The residue was dissolved in ethyl acetate (200 mL) and washed with H₂O (150 mL), dried over MgSO₄, filtered and concentrated to dryness. The product was recrystallized from ethyl acetate/diethyl ether to yield the desired N-hydroxy-5-norbornene-2,3-dicarboxyl-imido)-N-PNZ-4-amino-2(S)-hydroxy-butanoate (10.0 g, 21.78 mmol, 72.6% yield). MS m/e [M+H]⁺ Calcd 482.1. Found 482.2.

### (N-Hydroxy-5-norbornene-2,3-dicarboxyl-imido)-N-PNZ-4-amino-2(R)-benzoyl-butanoate

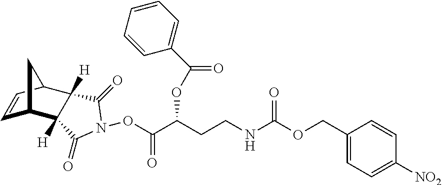

To a stirring solution of (N-hydroxy-5-norbornene-2,3-dicarboxyl-imido)-N-PNZ-4-amino-2(S)-hydroxy-butanoate (6.4 g, 0.014 mol) in THF (65 mL) was added triphenyl phosphine (4.0 g, 0.015 mmol), followed by benzoic acid (1.9 g, 0.015 mmol) and the reaction mixture was cooled to 0° C. DIAD (3.0 mL, 0.015 mol) was then added dropwise, and the reaction mixture was stirred for an additional 50 min. Solvent evaporation gave a crude, which was purified by flash chromatography (silica gel/hexanes:ethyl acetate 20-100%) to yield the desired (N-hydroxy-5-norbornene-2,3-dicarboxyl-imido)-N-PNZ-4-amino-2(R)-benzoyl-butanoate (2.3 g, 4.08 mmol, 29.1% yield), with minor contamination with triphenyl phosphine oxide: ¹H NMR (400 MHz, CDCl₃) δ 8.17 (d, 2H), 7.98 (d, 2H), 7.44-7.70 (m, 5H), 5.96-6.18 (m, 2H), 5.41-5.55 (m, 1H), 5.10 (s, 2H), 3.40-3.58 (m, 2H), 3.21-3.39 (m, 4H), 2.10-2.22 (m, 2H), 1.44-1.60 (m, 2H).

### 6′-Trifluoroacetyl-2′,3-diPNZ-1-(N-PNZ-4-amino-2(R)—O-benzoyl-butyryl)-3″-Boc-sisomicin

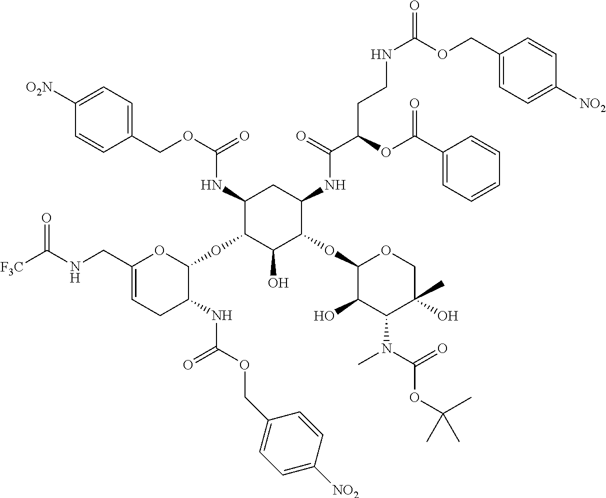

To a stirring solution of 6′-trifluoroacetyl-2′,3-diPNZ-sisomicin (2.5 g, 2.77 mmol) in DMF (50 mL) was added (N-hydroxy-5-norbornene-2,3-dicarboxyl-imido)-N-PNZ-4-amino-2(R)-benzoyl-butanoate (2.3 g, 4.08 mmol) and the reaction was stirred for 24 hr. DIPEA (2.5 mL, 0.014 mol) was then added, followed by Boc anhydride (2.5 mL, 0.011 mol) and the reaction mixture was stirred for an additional 2 hr. A solution of glycine (2.5 g, 0.033 mol) and K₂CO₃ (4.6 g, 0.033 mol) in H₂O (50 mL) was then added in portions over 5 minutes, and the reaction mixture was stirred for 1 hour. The reaction mixture was diluted with ethyl acetate (300 mL) and the aqueous layer was separated. The organic layer was washed with 1M citric acid (150 mL), sat. aq. NaHCO₃ (30 mL), brine (30 mL), dried over MgSO₄, filtered and concentrated to dryness to yield a crude, which was purified by RP HPLC Method 2-Column B to yield the desired 6′-trifluoroacetyl-2′,3-diPNZ-1-(N-PNZ-4-amino-2(R)—O-benzoyl-butyryl)-3″-Boc-sisomicin (1.6 g, 1.15 mmol, 41.5% yield).

### 2′,3-diPNZ-1-(N-PNZ-4-amino-2(R)-hydroxy-butyryl)-3″-Boc-sisomicin

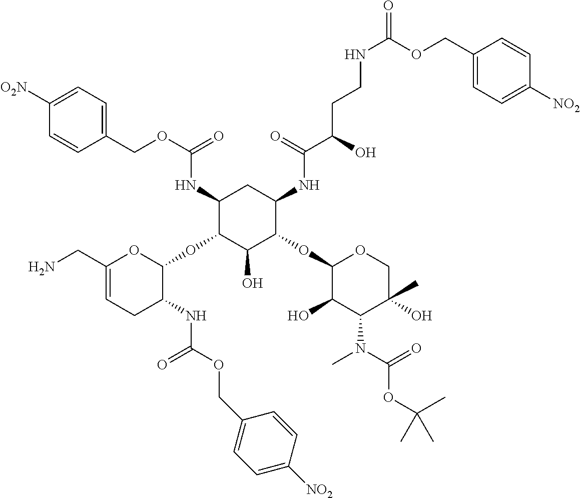

To a stirring solution of 6′-Trifluoroacetyl-2′,3-diPNZ-1-(N-PNZ-4-amino-2(R)—O-benzoyl-butyryl)-3″-Boc-sisomicin (1.6 g, 1.15 mmol) in MeOH (30 mL) was added conc. NH₄OH (3 mL) and the reaction was stirred for 3 days. Ethyl acetate (30 mL) was then added and the aqueous layer was separated. The organic layer was washed with 1M NaOH (20 mL), brine (20 mL), dried over MgSO₄, and concentrated to dryness to yield 2′,3-diPNZ-1-(N-PNZ-4-amino-2(R)-hydroxy-butyryl)-3″-Boc-sisomicin (1.4 g, MS m/e [M+H]⁺ Calcd 1186.4. Found 1186.2, [M+Na]⁺ 1208.3), which was carried throught to the next step without further purification.

### (R)-Ethyl 3-azido-2-hydroxypropionate

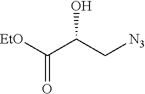

Ethyl-(2R)-2,3-epoxyproprionate (0.5 g, 4.3 mmol), ammonium chloride (0.253 g, 4.73 mmol), and sodium azide (0.336 g, 5.17 mmol) were combined in DMF (8 mL), and the mixture was heated at 75° C. for 14 hours. The reaction was cooled to room temperature, and was partitioned between water and ether/hexanes (1:1 v/v). The phases were separated, and the organic phase was washed once each with water, brine, dried over MgSO₄, filtered, and concentrated to an oil, which was purified by flash chromatography (silica gel/hexanes:10% ethyl acetate) to give (R)-ethyl-3-azido-2-hydroxypropionate as a clear oil (0.47 g, 2.97 mmol, 69% yield). Rf 0.27 (hexanes: 10% EtOAc, v/v, p-anisaldehyde); MS m/e [M+Na]⁺ Calcd 182.1. Found 182.0.

### (R)-3-(tert-Butoxycarbonylamino)-2-hydroxypropionic acid

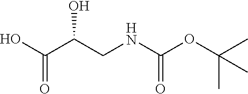

Step 1) To a stirring solution of (R)-ethyl-3-azido-2-hydroxypropionate (159 mg, 1.0 mmol) in ethanol (4 mL) was added acetic acid (0.10 mL), followed by 5% Pd/C (25 mg) after the flask had been flushed with nitrogen. The flask was fitted with a balloon of hydrogen, and stirred for 1 hour. The flask was then flushed with nitrogen, the mixture was filtered through Celite, and the pad was rinsed with ethanol (4 mL).

Step 2) To the filtrate was added 1M NaOH (3 mL), followed by Boc₂O (0.28 mL, 0.27 g, 1.2 mmol), and the solution was stirred at room temperature for 2 days. The solution was then partitioned between ether and water, and the phases were separated. The aqueous phase was washed twice with ether, acidified with 1M NaHSO₄, and extracted with ethyl acetate. The ethyl acetate phase was washed with brine, dried over MgSO₄, filtered, and concentrated to an oil, which solidified to give (R)-3-(tert-butoxycarbonylamino)-2-hydroxypropionic acid (117 mg, 57% yield): Rf 0.22 (CHCl₃:10% IPA, 1% AcOH, ninhydrin).

### 6′-Trifluoroacetyl-2′,3-di-PNZ-1-[(R)-3-(tert-butoxycarbonylamino)-2-hydroxy-propionyl]-sisomicin

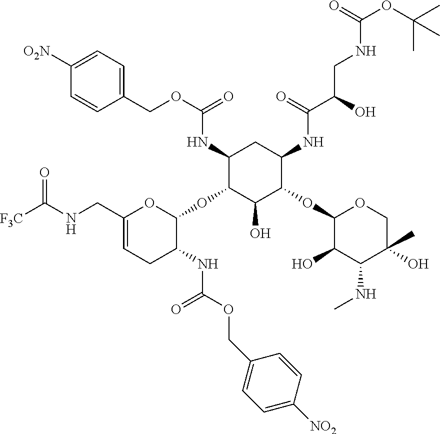

(R)-3-(tert-Butoxycarbonylamino)-2-hydroxypropionic acid (1.3 g, 6.3 mmol) and HONB (1.35 g, 7.5 mmol) were dissolved in THF (40 mL), the solution was cooled to 0° C., and EDC (1.33 g, 6.9 mmol) was added. After 20 minutes the reaction was allowed to warm to room temperature. After 6 hours, a solution of 6′-trifluoroacetyl-2′,3-di-PNZ-sisomicin (5.23 g, 5.8 mmol) in DMF (25 mL) was added, and the solution was allowed to stir overnight. The reaction was concentrated to remove the THF, and was partitioned between water and ethyl acetate. The phases were separated, and the ethyl acetate phase was washed once each with water, sat. NaHCO₃, water, and brine. The ethyl acetate phase was then dried over Na₂SO₄, filtered, and concentrated to a residue. The residue was chromatographed by RP HPLC Method 2-Column B to give 6′-trifluoroacetyl-2′,3-di-PNZ-1-[(R)-3-(tert-butoxycarbonylamino)-2-hydroxy-propionyl]-sisomicin as an off-white foam (1.64 g, 1.51 mmol, 24% yield): MS m/e [M+H]⁺ Calcd 1089.4. Found 1089.2.

### 6′-Trifluoroacetyl-2′,3-di-PNZ-1-[(R)-3-(tert-butoxycarbonylamino)-2-hydroxy-propionyl]-3″-Boc-sisomicin

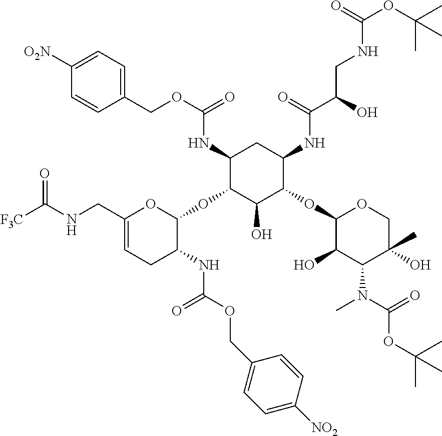

To a stirring solution of 6′-trifluoroacetyl-2′,3-diPNZ-1-[(R)-3-(tert-butoxycarbonylamino)-2-hydroxy-propionyl]-sisomicin (1.52 g, 1.39 mmol) in THF (10 mL) and methanol (5 mL) was added Boc₂O (0.65 mL, 0.62 g, 2.8 mmol). After three hours, glycine (312 mg, 4.17 mmol) and 0.5M K₂CO₃ (24 mL) were added, and the reaction was stirred vigorously for one hour. The mixture was then partitioned between ethyl acetate and water, and the phases were separated. The ethyl acetate phase was washed once each with water and brine, dried over MgSO₄, filtered, and concentrated to dryness to give 6′-trifluoroacetyl-2′,3-diPNZ-1-[(R)-3-(tert-butoxycarbonylamino)-2-hydroxy-propionyl]-3″-Boc-sisomicin as a solid that was carried through to the next step without further purification. MS m/e [M-Boc]⁺ Calcd 1089.4. Found 1089.2.

### 2′,3-diPNZ-1-[(R)-3-(tert-butoxycarbonylamino)-2-hydroxy-propionyl]-3″-Boc-sisomicin

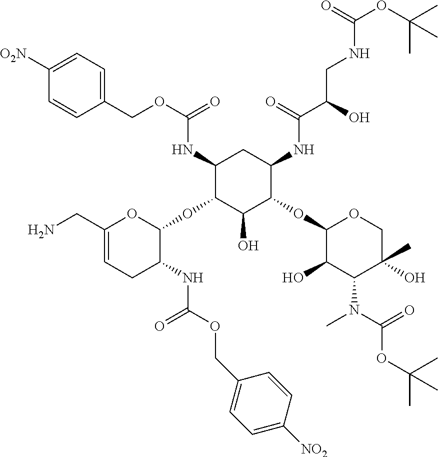

To a solution of 6′-trifluoroacetyl-2′,3-diPNZ-1-[(R)-3-(tert-butoxycarbonylamino)-2-hydroxy-propionyl]-3″-Boc-sisomicin (1.39 mmol) in methanol (45 mL) was added concentrated ammonium hydroxide (45 mL, ˜12M). The solution was allowed to sit at ambient temperature for 18 hours, and was then concentrated in vacuo. The residue was partitioned between ethyl acetate and water, and the phases were separated. The water phase was back-extracted once with ethyl acetate. The combined ethyl acetate phases were concentrated to give a residue, which was dissolved in a 1:1:1 v/v mixture of methanol/acetic acid/water, and was purified by RP HPLC Method 2-Column B. The pure fractions were combined, basified with 1M Na₂CO₃, and were concentrated in vacuo to remove the acetonitrile. The mixture was then extracted twice with ethyl acetate. The final ethyl acetate phases were combined, washed with brine, dried over MgSO₄, filtered, and concentrated to give 2′,3-diPNZ-1-[(R)-3-(tert-butoxycarbonylamino)-2-hydroxy-propionyl]-3″-Boc-sisomicin (316 mg, 30% yield) as a white solid. MS m/e [M+H]⁺ Calcd 1093.4. Found 1093.3.

### N-Boc-3-amino-2(S)-hydroxy-propionic acid

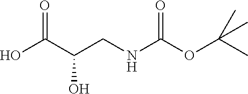

To a stirring solution of S-isoserine (4.0 g, 0.038 mol) in dioxane: H₂O (100 mL, 1:1 v/v) at 0° C. was added N-methylmorpholine (4.77 mL, 0.043 mol), followed by Boc₂O (11.28 mL, 0.049 mol) and the reaction was stirred overnight with gradual warming to room temperature. Glycine (1.0 g, 0.013 mol) was then added and the reaction was stirred for 20 min. The reaction was cooled to 0° C. and sat aq. NaHCO₃ (75 mL) was added. The aqueous layer was washed with ethyl acetate (2×60 mL) and then acidified to pH 1 with NaHSO₄. This solution was then extracted with ethyl acetate (3×70 mL) and these combined organic layers were dried over Na₂SO₄, filtered and concentrated to dryness to give the desired N-Boc-3-amino-2(S)-hydroxy-propanoic acid (6.30 g, 0.031 mmol, 81.5% yield): ¹H NMR (400 MHz, CDCl₃) δ 7.45 (bs, 1H), 5.28 (bs, 1H), 4.26 (m, 1H), 3.40-3.62 (m, 2H), 2.09 (s, 1H), 1.42 (s, 9H); ¹³C NMR (100 MHz, CDCl₃) δ 174.72, 158.17, 82, 71.85, 44.28, 28.45.

### 6′-Trifluoroacetyl-2′,3-diPNZ-1-(N-Boc-3-amino-2(S)-hydroxy-propionyl)-sisomicin

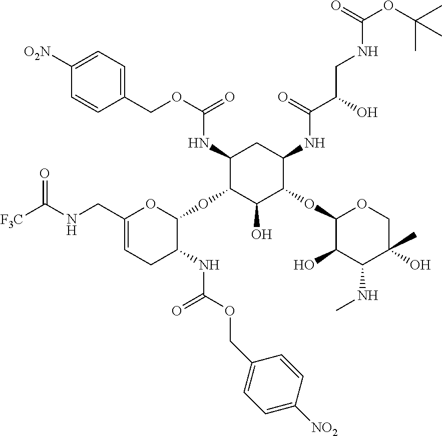

To a stirring solution of N-Boc-3-amino-2(S)-hydroxy-propionic acid (1.30 g, 6.34 mmol) in DMF (14 ml) was slowly added HONB (1.14 g, 6.34 mmol) and EDC (1.21 g, 6.34 mmol) and the reaction mixture was stirred for 2 hours, when MS showed complete formation of the activated ester (MS m/e [M+Na]⁺ Calcd 389.1. Found 389.1). 6′-trifluoroacetyl-2′,3-diPNZ-sisomicin (4.76 g, 5.28 mmol) was then added and the reaction was allowed to stir overnight. The reaction was quenched with sat. aq. NaHCO₃ (10 ml) and was extracted with EtOAc (5×15 mL). The combined organic layers were dried over Na₂SO₄, filtered and evaporated to dryness to yield a crude, which was purified by RP HPLC Method 2-Column B to yield the desired 6′-trifluoroacetyl-2′,3-diPNZ-1-(N-Boc-3-amino-2(S)-hydroxy-propionyl)-sisomicin (1.66 g, 1.52 mmol, 29% yield, >95% purity): MS m/e [M+H]⁺ Calcd 1089.4. Found 1089.2, [M+Na]⁺ 1111.3.

### 6′-Trifluoroacetyl-2′,3-diPNZ-1-(N-Boc-3-amino-2(S)-hydroxy-propionyl)-3″-Boc-sisomicin

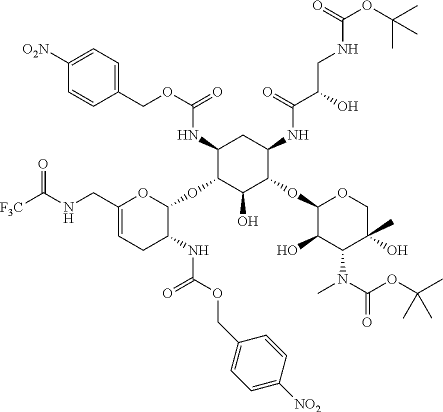

To a stirring suspension of 6′-trifluoroacetyl-2′,3-diPNZ-1-(N-Boc-3-amino-2(S)-hydroxy-propionyl)-sisomicin (1.66 g, 1.52 mmol) in MeOH (20 mL) at 0° C. was added DIPEA (0.53 mL, 3.05 mmol) followed by Boc-anhydride (0.52 mL, 2.29 mmol) and the reaction was allowed to warm to room temperature. After 2 hours everything had gone into solution. The reaction was cooled to 0° and quenched with glycine (0.5 g, 6.66 mmol) and sat. aq. NaHCO₃. The reaction was extracted with EtOAc (3×20 mL) and the combined organic layers were dried over Na₂SO₄, filtered and evaporated to dryness to yield 6′-trifluoroacetyl-2′,3-diPNZ-1-(N-Boc-3-amino-2(S)-hydroxy-propionyl)-3″-Boc-sisomicin (MS m/e [M+H]⁺ Calcd 1189.4. Found 1188.8, [M+Na]⁺ 1211.3), which was used in the next step without further purification.

### 2′,3-diPNZ-1-(N-Boc-3-amino-2(S)-hydroxy-propionyl)-3″-Boc-sisomicin

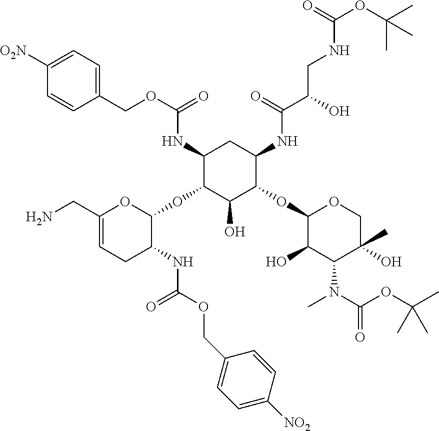

6′-Trifluoroacetyl-2′,3-diPNZ-1-(N-Boc-3-amino-2(S)-hydroxy-propionyl)-3″-Boc-sisomicin (1.52 mmol) was dissolved in MeOH (12 mL) and conc. NH₄OH (20 mL) was added, and the reaction was stirred overnight. Solvent evaporation gave a crude, which was purified by RP HPLC Method 2-Column B to yield the desired 2′,3-diPNZ-1-(N-Boc-3-amino-2(S)-hydroxy-propionyl)-3″-Boc-sisomicin (0.96 g, 0.79 mmol, 51.9% yield, >95% purity): MS m/e [M+H]⁺ Calcd 1093.4. Found 1093.2, [M+Na]^{+1115.3.}

### 6′-Trifluoroacetyl-2′,3-diPNZ-1-(N-PNZ-4-amino-2(S)-hydroxy-butyryl)-sisomicin

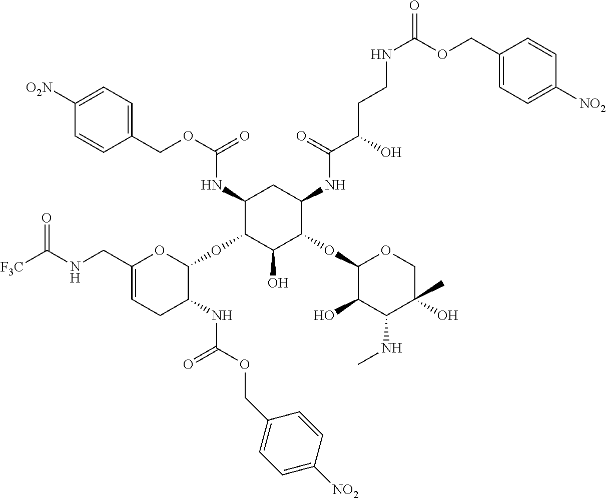

To a stirring solution of N-PNZ-4-amino-2(S)-hydroxy-butiric acid (1.47 g, 4.9 mmol) in DMF (50 ml) was slowly added HONB (0.884 g, 4.9 mmol) and EDC (0.945 g, 4.9 mmol) and the reaction mixture was stirred for 2 hours. 6′-Trifluoroacetyl-2′,3-diPNZ-sisomicin (3.42 g, 3.8 mmol) was then added and the reaction was allowed to stir overnight. The reaction was quenched with sat. aq. NaHCO₃ (30 ml) and was extracted with EtOAc (5×50 mL). The combined organic layers were dried over MgSO₄, filtered and concentrated to yield the desired 6′-trifluoroacetyl-2′,3-diPNZ-1-(N-PNZ-3-amino-2(S)-hydroxy-butyryl)-sisomicin (MS m/e [M+H]⁺ 1182.4. Found 1182.4), which was carried through to the next step without further purification.

### 6′-Trifluoroacetyl-2′,3-diPNZ-1-(N-PNZ-4-amino-2(S)-hydroxy-butyryl)-3″-Boc-sisomicin

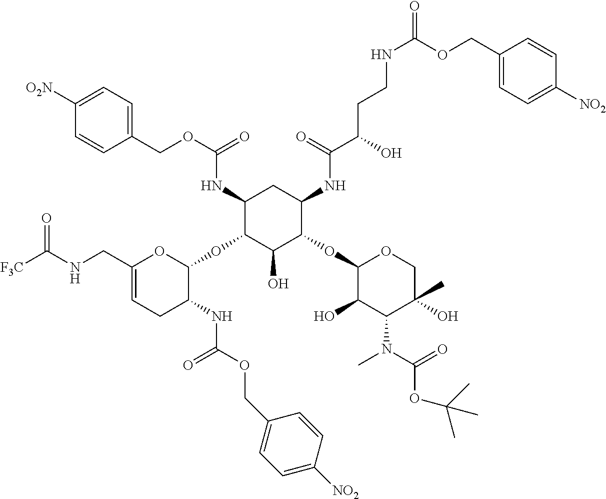

To a stirring solution of 6′-trifluoroacetyl-2′,3-diPNZ-1-(N-PNZ-3-amino-2(S)-hydroxy-butyryl)-sisomicin (4.9 mmol) in MeOH (50 mL) at 0° C. was added DIPEA (1.70 mL, 9.8 mmol), followed by Boc anhydride (1.6 g, 7.35 mmol) and the reaction was allowed to warm to room temperature. The reaction was then cooled to 0° C. and quenched with glycine (1.10 g, 14.7 mmol) and sat. aq. NaHCO₃. The reaction was extracted with EtOAc (3×50 mL) and the combined organic layers were dried over MgSO₄, filtered and evaporated to dryness to yield 6′-trifluoroacetyl-2′,3-diPNZ-1-(N-PNZ-4-amino-2(S)-hydroxy-butyryl)-3″-Boc-sisomicin, which was used in the next step without further purification.

### 2′,3-diPNZ-1-(N-PNZ-4-amino-2(S)-hydroxy-butyryl)-3″-Boc-sisomicin

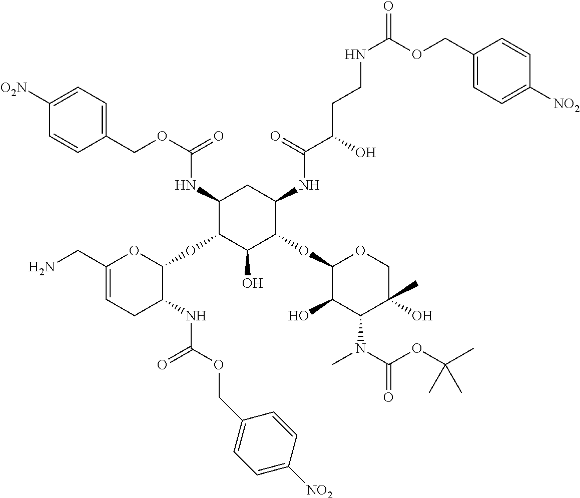

6′-Trifluoroacetyl-2′,3-diPNZ-1-(N-Boc-3-amino-2(S)-hydroxy-butyryl)-3″-Boc-sisomicin (4.9 mmol) was dissolved in MeOH (30 mL) and conc. NH₄OH (50 mL) was added, and the reaction was stirred overnight. Solvent evaporation gave a crude, which was purified by RP HPLC Method 2-Column B to yield the desired product 2′,3-diPNZ-1-(N-PNZ-4-amino-2(S)-hydroxy-butyryl)-3″-Boc-sisomicin. MS m/e [M+H]⁺ Calcd 1186.4. Found 1186.3.

### 6′-PNZ-sisomicin

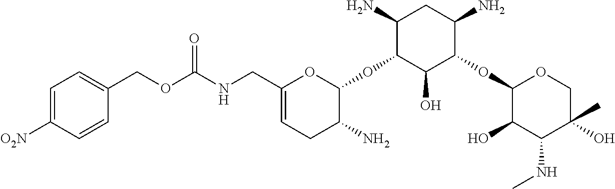

To a stirring solution of sisomicin (19.1 g, 42.65 mmol) in MeOH (300 mL) was added Zn(OAc)₂ (23.5 g, 0.128 mol) and the reaction mixture was stirred for 1 hour until all the zinc had gone into solution. A solution of (N-hydroxy-5-norbornene-2,3-dicarboxyl-imido)-4-nitro-benzoate (15.28 g, 42.65 mmol) in DCM (150 mL) was then added dropwise over 3 hours and the reaction was allowed to stir overnight. The reaction was then concentrated to dryness to yield a crude, which was slowly added to a vigorously stirring solution of 10% aq NH₄OH (480 mL) and DCM (180 mL). The aqueous layer was separated, washed with DCM (3×160 mL), and diluted with brine (250 mL). The aqueous layer was extracted with DCM: IPA (7:3 v/v, 4×160 mL). The combined organic layers were washed with 10% aq. NH₄OH: brine (7:3 v/v, 200 mL), dried over MgSO₄, filtered and concentrated to yield the desired 6′-PNZ-sisomicin: MS m/e [M+H]⁺ Calcd 627.3. Found 627.2; CLND 95% purity.

### (N-Hydroxy-5-norbornene-2,3-dicarboxyl-imido)-tert-butyl-carbonate

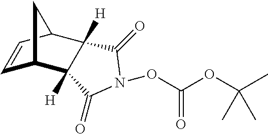

To a stirring solution of N-hydroxy-5-norbornene-2,3-dicarboximide (20.0 g, 0.112 mol) in THF (200 mL) at 0° C. was added triethylamine (0.65 mL, 4.8 mmol), followed by the dropwise addition of a solution of Boc₂O (29.23 g, 0.134 mol) in THF (30 mL) and the reaction was allowed to stir overnight with gradual warming to room temperature. A precipitate formed, which was filtered and washed with cold THF (200 mL). The crude solid was then vigorously stirred in MeOH (100 mL) for 1 hour, before being filtered, washed with MeOH (50 mL), and dried under high vacuum to yield the desired (N-hydroxy-5-norbornene-2,3-dicarboxyl-imido)-tert-butylcarbonate as a white solid (28.0 g, 0.1 mol, 89.3% yield): TLC (hexanes: ethyl acetate, 1:1 v/v) Rf=0.44; NMR (400 MHz, DMSO-d₆) δ 6.10 (bs, 2H), 3.48 (bs, 2H), 3.29-3.32 (m, 2H), 1.58-1.62 (m, 1H), 1.50-1.55 (m, 1H), 1.47 (s, 9H).

### 6′-PNZ-2′,3-diBoc-sisomicin

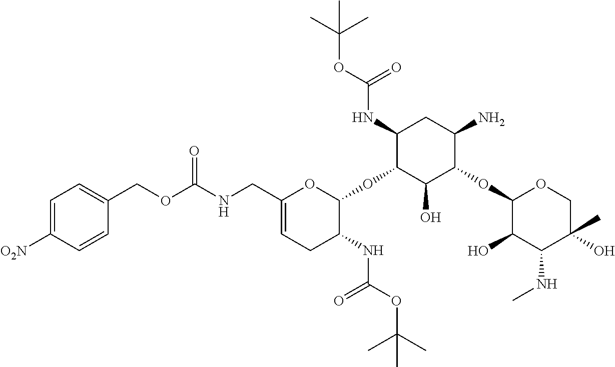

To a stirring solution of 6′-PNZ-sisomicin (5.86 g, 9.35 mmol) in MeOH (100 mL) was added Zn(OAc)₂ (5.15 g, 28.05 mmol) and the reaction mixture was stirred for 1 hour until all solids had dissolved. A solution of (N-hydroxy-5-norbornene-2,3-dicarboxyl-imido)-tert-butylcarbonate (4.96 g, 17.77 mmol) in THF (48 mL) was added dropwise over 4 hours and the reaction mixture was allowed to stir overnight. Triethylamine (2.61 ml, 18.7 mmol) was then added, followed by a solution of (N-hydroxy-5-norbornene-2,3-dicarboxyl-imido)-tert-butylcarbonate (1.31 g, 4.68 mmol) in THF (12 mL) and the reaction mixture was stirred for an additional 24 hours. The reaction was quenched by the addition of glycine (2.81 g, 37.4 mmol). The solvent was removed by rotary evaporation to yield a residue, which was dissolved in DCM (200 mL) and washed with H₂O: conc. NH₄OH (7:3 v/v, 3×50 mL). The organic layer was dried over MgSO₄, filtered and concentrated to dryness. The solids were dissolved in 0.1M aq AcOH (2.0 L) and washed with ethyl acetate: diethyl ether (9:1 v/v, 4×1.0 L). The aqueous layer was then basified to pH 10 with conc. NH₄OH, salted and extracted with ethyl acetate (3×30 mL). The combined organic layers were dried over MgSO₄, filtered and concentrated to yield 6′-PNZ-2′,3-diBoc-sisomicin (4.1 g, 4.96 mmol, 53.0% yield, 92% purity): MS m/e [M+H]⁺ Calcd 827.4. Found 827.2.

### (N-Hydroxy-5-norbornene-2,3-dicarboxyl-imido)-9-fluorene-acetate

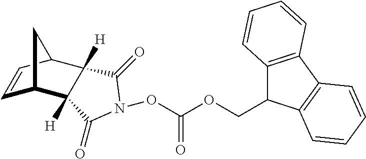

To a stirring solution of N-hydroxy-5-norbornene-2,3-dicarboximide (7.38 g, 0.041 mol) in THF (200 mL) at 0° C. was added N-methylmorpholine (4.53 mL, 0.041 mol), followed by the dropwise addition of a solution of 9-fluorenylmethyl chloroformate (10.15 g, 0.039 mol) in THF (50 mL), and the reaction was stirred overnight with gradual warming to room temperature. The flask was then cooled to 0° C. and the precipitated salts were removed by filtration. The filtrate was concentrated under vacuum to yield a waxy residue, which was precipitated from methanol to yield (N-hydroxy-5-norbornene-2,3-dicarboxyl-imido)-9-fluorene-acetate (9.9 g, 0.025 mol, 61.0% yield), which was carried through to the next step without further purification: TLC (hexanes:ethyl acetate 3:1 v/v) R_{f}=0.28.

### 6′-PNZ-2′,3,3″-triBoc-1-Fmoc-sisomicin

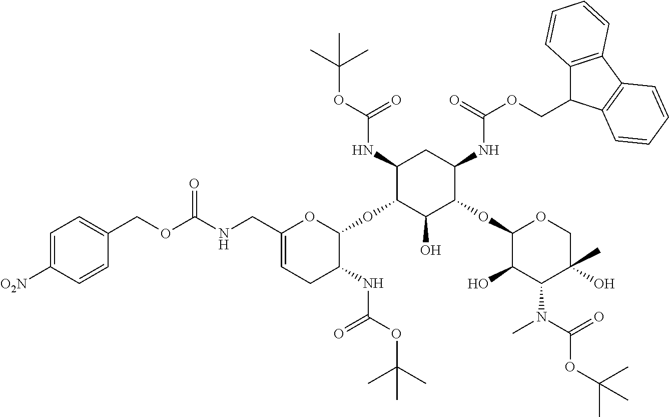

To a stirring solution of 6′-PNZ-2′,3-diBoc-sisomicin (7.38 g, 8.93 mmol) in THF (200 mL) was added (N-hydroxy-5-norbornene-2,3-dicarboxyl-imido)-9-fluorene-acetate (2.51 g, 6.25 mmol), and the reaction was allowed to stir for 1 hour with its progress monitored by HPLC and MS (MS m/e [M+H]⁺ Calcd 1049.5. Found 1049.4. Additional (N-hydroxy-5-norbornene-2,3-dicarboxyl-imido)-9-fluoree-acetate (0.05 eq) was added and the reaction was stirred for 1.5 hours. N-Methylmorpholine (0.98 ml, 8.93 mmol) was then added followed by the addition of Boc anhydride (3.94 g, 17.85 mmol), and the reaction was stirred for 3 hours. The reaction was quenched by the addition of glycine (7.51 g, 40.18 mmol) and was allowed to stir overnight. The precipitated salts were filtered and the resulting solution was concentrated to dryness to yield a residue, which was dissolved in DCM (150 mL) and washed with sat. aq. NaHCO₃ (3×80 mL), 1M citric acid (3×80 mL), H₂O: NaHCO₃ (1:1 v/v, 80 mL), brine (40 mL) and dried over MgSO₄. Filtration and solvent evaporation gave the desired 6′-PNZ-2′,3,3″-triBoc-1-Fmoc-sisomicin (MS m/e [M+Na]⁺ Calcd 1171.5. Found 1171.3), which was carried through to the next step without further purification.

### 6′-PNZ-2′,3,3″-triBoc-sisomicin

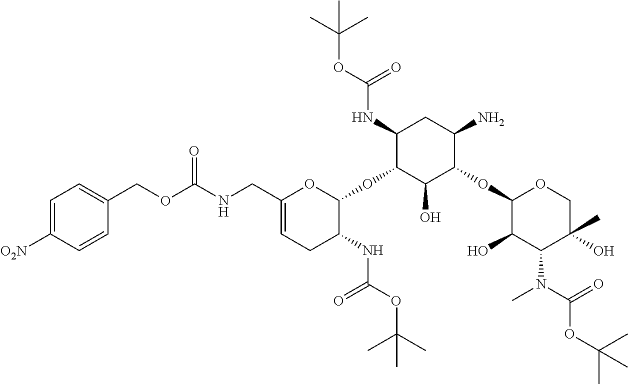

To a stirring solution of 6′-PNZ-2′,3,3″-triBoc-1-Fmoc-sisomicin (8.93 mmol) in DCM (150 mL) was slowly added tris(2-aminoethyl)amine (13.37 mL, 89.27 mmol) and the reaction was stirred for 45 min. The reaction mixture was then washed with brine (3×100 mL), a pH 5.5 phosphate buffered solution (2×500 mL, 1×100 mL), H₂O (100 mL), sat. aq. NaHCO₃ (100 mL), and brine (100 mL). The organic phase was concentrated to yield a crude, which was purified by RP HPLC Method 2-Column B to yield the desired 6′-PNZ-2′,3,3″-triBoc-sisomicin (2.77 g, 2.99 mmol, 33.5% yield, 93% purity): MS m/e [M+H]⁺ Calcd 927.4. Found 927.2.

### 6′-PNZ-2′,3,3″-triBoc-1-(N-Boc-3-amino-2(S)-hydroxy-propionyl)-sisomicin

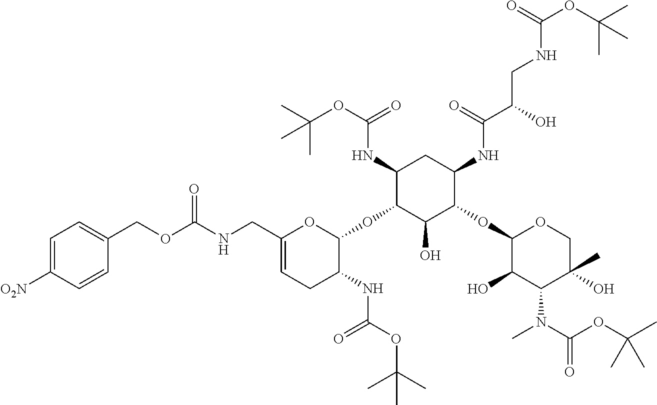

To a stirring solution of N-Boc-3-amino-2(S)-hydroxy-propionic acid (0.93 g, 4.53 mmol) in DMF (8 ml) was slowly added HONB (0.82 g, 4.53 mmol) and EDC (0.87 g, 4.53 mmol) and the reaction mixture was stirred for 2 hours. 6′-PNZ-2′,3,3″-triBoc-sisomicin (3.0 g, 3.23 mmol) was then added and the reaction was allowed to stir overnight. The reaction was quenched with H₂O (10 ml) and was extracted with EtOAc (5×15 mL). The combined organic layers were dried over Na₂SO₄, filtered and concentrated to dryness to give the desired 6′-PNZ-2′,3,3″-triBoc-1-(N-Boc-3-amino-2(S)-hydroxy-propionyl)-sisomicin (MS m/e [M+H]⁺ Calcd 1114.5. Found 1113.9, [M+Na]⁺ 1136.3), which was carried through to the next step without further purification.

### 2′,3,3″-triBoc-1-(N-Boc-3-amino-2(S)-hydroxy-propionyl)-sisomicin

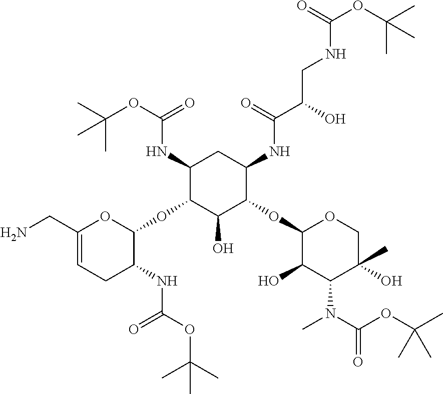

6′-PNZ-2′,3,3″-triBoc-1-(N-Boc-3-amino-2(S)-hydroxy-propionyl)-sisomicin (3.23 mmol) was submitted to Procedure 2 for PNZ removal to yield 2′,3,3″-triBoc-1-(N-Boc-3-amino-2(S)-hydroxy-propionyl)-sisomicin (2.0 g, 2.14 mmol, 66.2% yield, purity >65%): MS m/e [M+H]⁺ Calcd 935.5. Found 935.3, [M+Na]⁺ 957.3.

### N-Boc-4-amino-2(S)-hydroxy-butyric acid

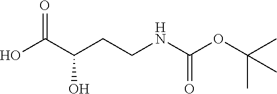

To a stirring solution of S-4-amino-2-hydroxy-butyric acid (51.98 g, 0.44 mol) in dioxane: H₂O (2 L, 1:1 v/v) was added K₂CO₃ (106 g, 0.91 mol) followed by a solution of Boc-anhydride (100 g, 0.46 mol) in dioxane (100 mL), and the reaction was stirred overnight. The reaction was washed with DCM (2×300 mL), and the aqueous layer was acidified to pH 2 with H₃PO₄. The aqueous layer was extracted with DCM (2×300 mL), and the combined organic layers were dried over MgSO₄, filtered and concentrated to dryness to yield the desired N-Boc-4-amino-2(S)-hydroxybutyric acid (48.2 g, 50% yield).

### 6′-PNZ-2′,3,3″-triBoc-1-(N-Boc-4-amino-2(S)-hydroxy-butyryl)-sisomicin

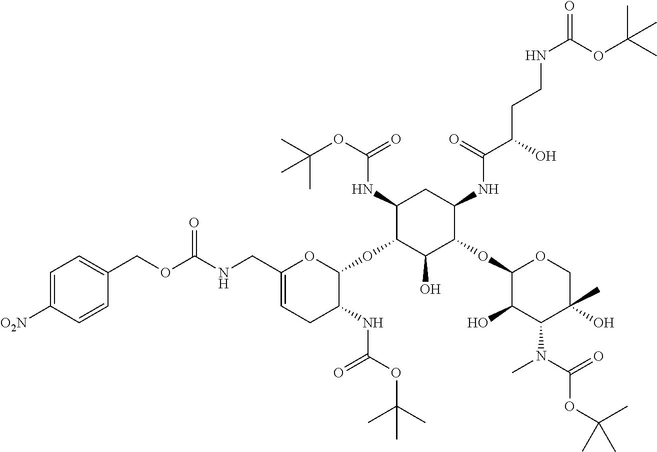

To a stirring solution of N-Boc-4-amino-2(S)-hydroxy-butyric acid (1.35 g, 6.17 mmol) in DMF (12 ml) was slowly added HONB (1.11 g, 6.17 mmol) and EDC (1.18 g, 6.17 mmol). A solution of 6′-PNZ-2′,3,3″-triBoc-sisomicin (4.4 g, 4.75 mmol) in DMF (13 mL) was then slowly added, and the reaction was allowed to stir overnight. The reaction was cooled to 0° C. and quenched with sat. aq. NaHCO₃ (20 mL) and was extracted with EtOAc (50 mL). The combined organic layers were washed with sat. aq. NaHCO₃ (2×20 mL), brine (25 mL), dried over MgSO₄, filtered and concentrated to dryness to give the desired 6′-PNZ-2′,3,3″-triBoc-1-(N-Boc-4-amino-2(S)-hydroxy-butyryl)-sisomicin (MS m/e [M+H]⁺ Calcd 1128.5. Found 1129.4), which was carried through to the next step without further purification.

### 2′,3,3″-triBoc-1-(N-Boc-4-amino-2(S)-hydroxy-butyryl)-sisomicin

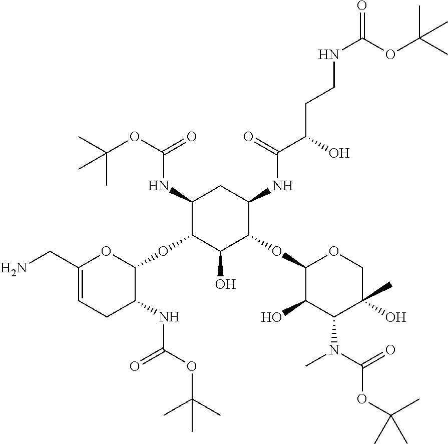

6′-PNZ-2′,3,3″-triBoc-1-(N-Boc-4-amino-2(S)-hydroxy-butyryl)-sisomicin (4.75 mmol) was submitted to Procedure 2 for PNZ removal to yield 2′,3,3″-triBoc-1-(N-Boc-4-amino-2(S)-hydroxy-butyryl)-sisomicin: MS m/e [M+H]⁺ Calcd 949.5. Found 949.1, [M+Na]⁺ 971.4.

### 6′,2′-diPNZ-sisomicin

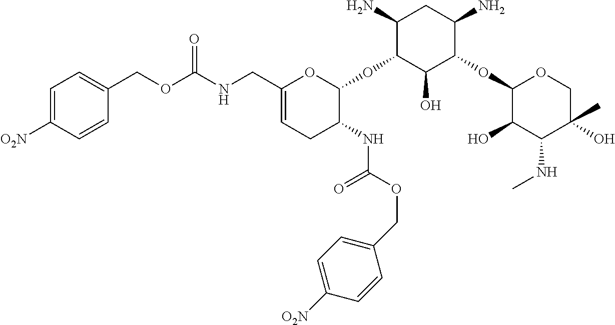

Sisomicin (12.9 g, 28.9 mmol) and Nickel (II) acetate (29 g, 115.6 mmol) were dissolved in methanol (900 ml), and the green solution was cooled in an ice-water bath. To this solution was added 2,4-dioxo-3-azabicyclo[3.2.1]oct-6-en-3-yl 4-nitrobenzyl carbonate (16.6 g, 46.2 mmol) as a solid. The mixture was allowed to slowly warm to room temperature and stir overnight. The solution was concentrated in vacuo to a green oil, and the oil was partitioned between concentrated ammonium hydroxide (˜12M) and ethyl acetate. The phases were separated, and the purple aqueous phase was back-extracted once with ethyl acetate. The combined ethyl acetate phases were washed once with brine, diluted with 10% by volume with isopropanol, and extracted three times with 5% aqueous acetic acid. The combined acetic acid phases were basified with 6M NaOH to pH>11, and were then extracted twice with ethyl acetate. The final two ethyl acetate phases were combined and washed once with brine, dried over Na₂SO₄, filtered, and concentrated to ½ volume in vacuo. The product precipitated during the concentration, and was isolated by filtration to give 6′,2′-di-PNZ-sisomicin (12.1 g, 65% yield) as a white solid. MS m/e [M+H]⁺ Calcd 806.3. Found 806.2.

### 6′,2′-diPNZ-1,3,3″-triBoc-sisomicin

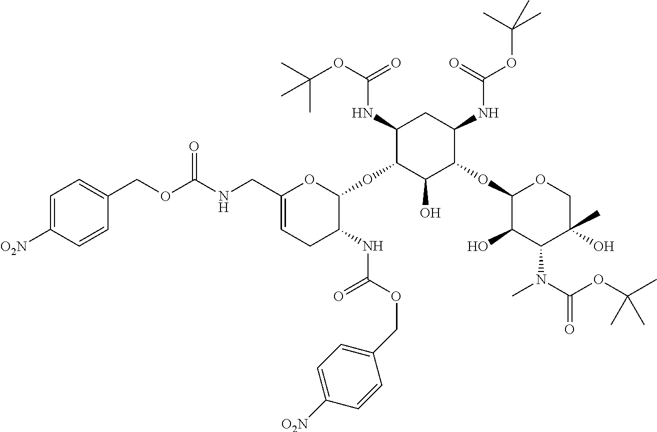

To a stirring solution of 6′,2′-diPNZ-sisomicin (4.1 g, 5.09 mmol) in THF (70 mL) and methanol (70 mL) with the flask placed in a water bath, was added di-tert-butyl-dicarbonate (5.8 mL, 5.51 g, 25.5 mmol). After 2 hours, glycine (1.9 g, 25.5 mmol), water (70 mL), and 1M sodium carbonate (15 mL) were added, and the mixture was stirred vigorously for 12 hours. The mixture was concentrated to remove the THF and methanol, and water (100 mL) was added to suspend the solids. The solids were isolated by filtration, washed with water, and dried to give 6′,2′-diPNZ-1,3,3″-triBoc-sisomicin (5.41 g, 96% yield) as a white solid. Rf 0.15 (CHCl₃:5% IPA v/v, UV) MS m/e [M-Boc]⁺ Calcd 1006.5. Found 1006.4.

### 1,3,3″-triBoc-sisomicin

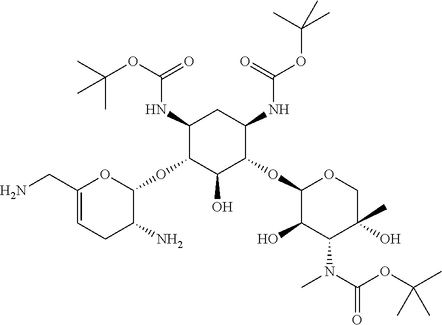

6′,2′-diPNZ-1,3,3″-triBoc-sisomicin (4.84 g, 4.38 mmol) and sodium hydrosulfite (7.6 g, 44 mmol) were combined with ethanol (70 mL) and water (70 mL) in a flask. The flask was fitted with a condenser, and the mixture was heated at 60° C. for 12 hours. The mixture was then heated at 65° C. for an additional three hours, followed by cooling to room temperature. The mixture was partitioned between 0.2 M NaOH and ethyl acetate, and the phases were separated. The aqueous phase was back-extracted once with ethyl acetate. The combined organic phases were washed once with brine, dried over Na₂SO₄, filtered, and concentrated to an oil. The oil was triturated with ether, and the solids were isolated by filtration to give 6′,2′-di-PNZ-1,3,3″-triBoc-sisomicin (2.71 g, 83% yield) as a white solid. Rf 0.23 (IPA: CHCl₃ 4:1, with 2% NH₃, UV, ninhydrin); MS m/e [M+H]⁺ Calcd 748.4. Found 748.3.

### 6′-PNZ-1,3,3″-triBoc-sisomicin

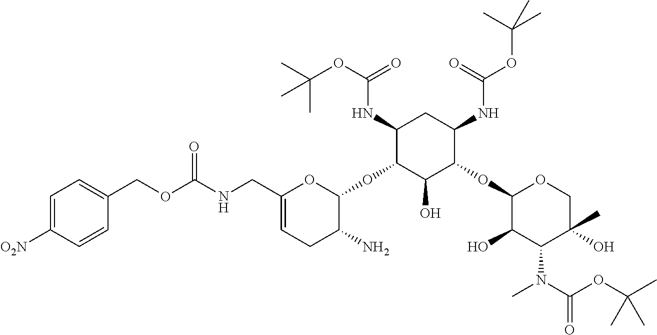

1,3,3″-triBoc-sisomicin (8.5 g, 11.4 mmol) was dissolved in methanol (212 mL) and cooled in an ice-water bath, and triethylamine (1.75 mL, 12.5 mmol) was added. 2,4-Dioxo-3-azabicyclo[3.2.1]oct-6-en-3-yl 4-nitrobenzyl carbonate (4.08 g, 11.4 mmol) was added as a solid. After 1 hour, the reaction was concentrated to a residue, which was partitioned between ether/ethyl acetate (1:1 v/v) and water. The phases were separated, and the organic phase was washed once with 5% aqueous acetic acid to remove the remaining starting material. The organic phase was then diluted with ⅓ volume of hexane, and was extracted three times with 5% aqueous acetic acid. These last three aqueous phases were combined, salted to approximately 10% saturation with NaCl, and were extracted twice with ethyl acetate. These last two ethyl acetate phases were combined, washed once each with 1M NaOH and brine, dried over Na₂SO₄, filtered, and concentrated. The resulting residue was triturated with ether/hexanes, and the solids were isolated by filtration to give 6′-PNZ-1,3,3″-triBoc-sisomicin (6.2 g, 61% yield) as a white solid. The unreacted starting material in the initial aqueous phase can be re-cycled by simply basifying the solution, extracting it into ethyl acetate, drying over Na₂SO₄, and concentrating. MS m/e [M+H]⁺ Calcd 927.4. Found 927.4.

### 6′,2′-diPNZ-3-Boc-sisomicin

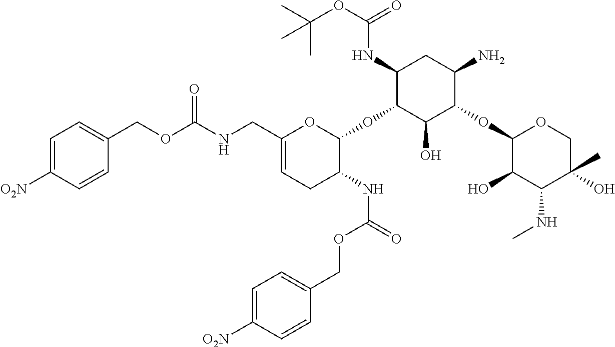

6′,2′-diPNZ-sisomicin (5.5 g, 6.8 mmol) and Zinc acetate (4.5 g, 20.4 mmol) were dissolved in methanol (200 mL) and the solution was cooled in an ice-water bath. tert-Butyl-2,4-dioxo-3-azabicyclo[3.2.1]oct-6-en-3-yl carbonate (1.9 g, 6.8 mmol, Boc-ONb) was added, and the reaction was allowed to warm slowly to room temperature and stir overnight. tert-Butyl-2,4-dioxo-3-azabicyclo[3.2.1]oct-6-en-3-yl carbonate (500 mg, ˜1.7 mmol) was added, and the solution was stirred for four hours. Another portion of tert-butyl-2,4-dioxo-3-azabicyclo[3.2.1]oct-6-en-3-yl carbonate (500 mg) was added, and the reaction was stirred for another four hours. The reaction was then concentrated to an oil, which was partitioned between concentrated ammonium hydroxide (˜12 M) and ethyl acetate, and the phases were separated. The ethyl acetate phase was washed once each with conc. ammonium hydroxide and water, and was then washed twice with 5% aqueous acetic acid that was 20% saturated with NaCl. The ethyl acetate phase was then diluted with 20% by volume hexanes, and was extracted with 5% aqueous acetic acid. The final acetic acid phase was basified with 6 M NaOH to pH >11, and was extracted once with fresh ethyl acetate. The final ethyl acetate phase was washed once with brine, dried over Na₂SO₄, filtered, and concentrated to an oil. The oil was dissolved in ethyl acetate (16 mL), and was dripped into ether (200 mL) to precipitate the product. The solids were isolated by filtration and washed with ether to give 6′,2′-di-PNZ-3-Boc-sisomicin (3.82 g, 62% yield) as a white solid. MS m/e [M+H]⁺ Calcd 906.4. Found 906.3.

### 6′,2′-diPNZ-3-Boc-1-(N-Boc-4-amino-2(S)-hydroxy-butyryl)-sisomicin

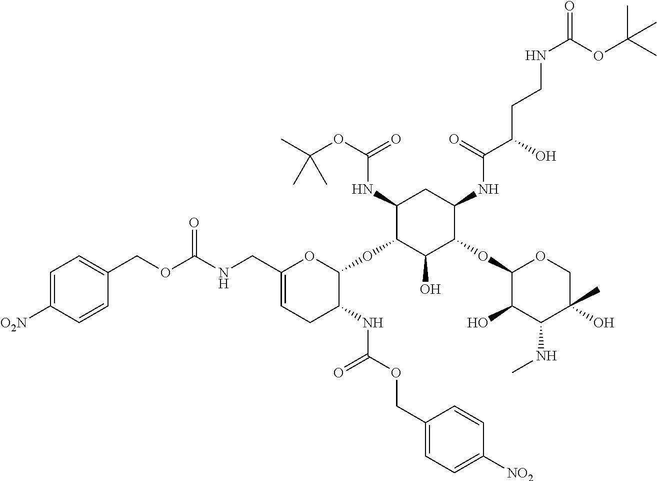

To a stirring solution of 6′,2′-diPNZ-3-Boc-sisomicin (10.0 g, 11.0 mmol) in DMF (100 mL) was added N-Boc-4-amino-2(S)-hydroxy-butyric acid (3.15 g, 14.4 mmol) and the reaction was cooled to −40° C. and stirred for 30 min. PyBOP (6.9 g, 13.2 mmol) was then added, followed by DIPEA (7.7 mL, 40.4 mmol) and the reaction was stirred for 3 hours at −40° C. The reaction was diluted with EtOAc (200 mL), and washed with water (2×100 mL). The aqueous layer was separated and extracted with EtOAc (100 mL). The combined organic layers were dried over Na₂SO₄, filtered and concentrated to yield 6′,2′-diPNZ-3-Boc-1-(N-Boc-4-amino-2(S)-hydroxy-butyryl)-sisomicin as a yellow-orange solid (HPLC 67% purity), which was carried through to the next step without further purification.

### 6′,2′-diPNZ-3,3″-diBoc-1-(N-Boc-4-amino-2(S)-hydroxy-butyryl)-sisomicin

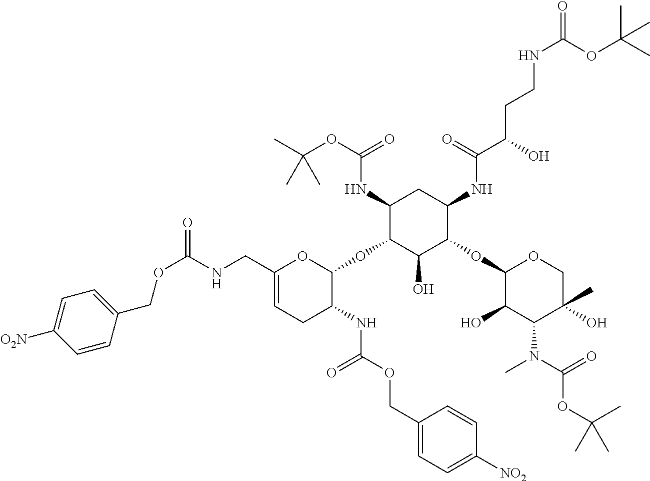

To a stirring solution of 6′,2′-diPNZ-3-Boc-1-(N-Boc-4-amino-2(S)-hydroxy-butyryl)-sisomicin (11.0 mmol) in THF (100 mL) at 0° C. was added N-methyl morpholine (2.44 mL, 22.1 mmol), followed by Boc-anhydride (4.82 g, 22.1 mmol) and the reaction mixture was stirred for 18 h. The reaction mixture was concentrated to dryness to yield a crude, which was purified by flash chromatography (silica gel/dichloromethane: methanol 0-7%) to yield the desired 6′,2′-diPNZ-3,3″-diBoc-1-(N-Boc-4-amino-2(S)-hydroxy-butyryl)-sisomicin (10.47 g, 9.46 mmol, 86.0% yield, anal. HPLC 85% purity): MS m/e [M+Na]⁺ Calcd 1229.5. Found 1229.4.

### 3,3″-diBoc-1-(N-Boc-4-amino-2(S)-hydroxy-butyryl)-sisomicin

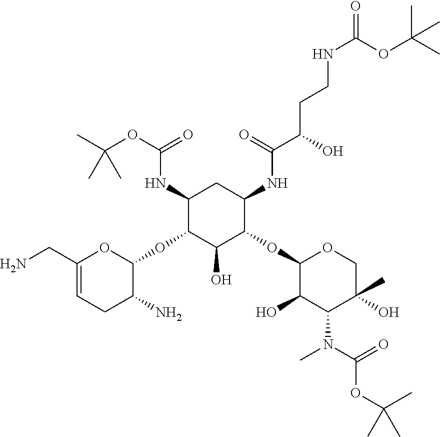

To a stirring solution of 6′,2′-diPNZ-3,3″-diBoc-1-(N-Boc-4-amino-2(S)-hydroxy-butyryl)-sisomicin (10.5 g, 8.71 mmol) in EtOH (100 mL) and H₂O (50 mL) was added 1M NaOH (34.8 ml, 34.8 mmol), followed by Na₂S₂O₄ (12.1 g, 69.6 mmol) and the reaction mixture was heated at 70° C. for 18 hours. Upon cooling, a precipitate formed, which was removed by filtration and washed with MeOH (25 mL). Removal of the organic solvents by rotary evaporation was followed by the addition of H₂O (100 mL) and acetic acid (200 mL) to obtain an acidic solution (pH˜4), which was washed with EtOAc (2×100 mL). The aqueous layer was then basified to pH 12 with conc. NH₄OH (20 mL), salted with NaCl (6.0 g) and extracted with EtOAc (2×200 mL). The combined organic layers were dried over Na₂SO₄, filtered, and concentrated to give the desired 3,3″-diBoc-1-(N-Boc-4-amino-2(S)-hydroxy-butyryl)-sisomicin (4.78 g, 5.45 mmol, 62.6% yield, MS m/e [M+H]⁺ Calcd 849.5. Found 849.3, [M+Na]⁺ 871.3), which was carried through to the next step without further purification.

### 6′-PNZ-3,3″-diBoc-1-(N-Boc-4-amino-2(S)-hydroxy-butyryl)-sisomicin

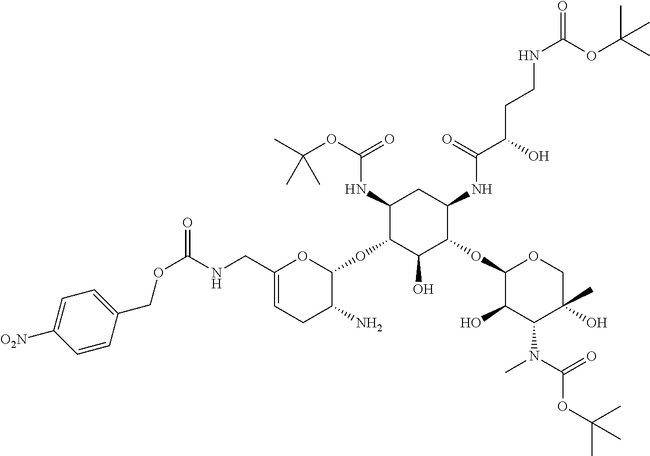

To a stirring solution of 3,3″-diBoc-1-(N-Boc-4-amino-2(S)-hydroxy-butyryl)-sisomicin (4.78 g, 5.45 mmol) in MeOH (75 mL) was added DIPEA (0.95 mL, 5.45 mmol), followed by (N-hydroxy-5-norbornene-2,3-dicarboxyl-imido)-4-nitro-benzyl carbonate (HONB-PNZ, 1.75 g, 4.90 mmol) and the reaction mixture was stirred for 1 hour. Solvent evaporation gave an oily residue, which was dissolved in EtOAc (100 mL), washed with H₂O (2×100 mL), and diluted with Et₂O (75 mL) and hexanes (50 mL). The organic layer was then extracted with 5% aq. AcOH (100 mL) and the aqueous layer was separated, salted with NaCl (3.0 g) and extracted with EtOAc (3×100 mL). The combined organic layers were dried over Na₂SO₄, filtered and concentrated to yield the desired 6′-PNZ-3,3″-diBoc-1-(N-Boc-4-amino-2(S)-hydroxy-butyryl)-sisomicin (3.08 g, 3.32 mmol, 60.9% yield; MS m/e [M+H]⁺ Calcd 1028.5. Found 1028.3; HPLC 90.0% purity), which was carried through to the next step without further purification.

### N-Boc-3-amino-propanal

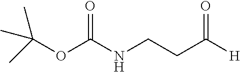

To a stirring solution of 3-(Boc-amino)-1-propanol (25 mL, 0.144 mol) in water saturated DCM (1.0 L) was added Dess-Martin reagent (99.2 g, 233.9 mmol) and the reaction mixture was stirred for 1 hour. The reaction was then diluted with ether (1.0 L), followed by a solution of Na₂S₂O₃ (250 g) in 80% NaHCO₃ (450 g in 1.0 L H₂O). The reaction was stirred vigorously for 30 minutes until two layers formed, the top layer was clear. The reaction was filtered to remove the precipitated solids and the aqueous layer was extracted with ether (1.0 L). The organic layer was washed with sat. NaHCO₃ (1.0 L), H₂O (1.0 L), and brine (1 L), dried over Na₂SO₄ and concentrated to a clear oil. The crude oil was dissolved in EtOAc:hexanes (1:1 v/v, 1.0 L) and filtered through a short silica gel column to yield the desired N-Boc-3-amino-propanal (21.7 g, 0.125 mol, 85.6% yield): ¹H NMR (400 MHz, CDCl₃) δ 9.77 (s, 1H, CHO), 4.85 (bs, 1 H, NH), 3.36-3.42 (m, 2H, CH₂), 2.67 (t, 2H, CH₂), 1.39 (s, 9H, (CH₃)₃).

### N-Boc-1-oxa-6-azaspiro[2.5]octane

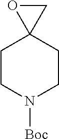

4-Methylene-piperidine (0.222 g, 1.12 mmol) was submitted to Procedure 14 to form the desired N-Boc-1-oxa-6-azaspiro[2.5]octane (0.215 g, 1.01 mmol, 90.2% yield): ¹H NMR (250 MHz, DMSO-d₆) δ 3.29-3.61 (m, 6H), 1.56-1.70 (m, 2H), 1.30-1.54 (m, 11H).

### 2-(Pent-4-enyl)-isoindoline-1,3-dione

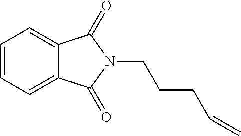

To a stirring solution of 5-bromo-pentene (6.0 g, 0.040 mol) in DMF (30 mL) was added K₂CO₃ (4.7 g, 0.034 mol) and potassium phthalimide (6.21 g, 0.033 mmol) and the reaction mixture was heated at 100° C. for 1 hr. The reaction mixture was cooled to room temperature, and water (50 mL) was added. The aqueous layer was then extracted with ethyl acetate (2×50 mL), and the combined organic layers were washed with 5% aq. NaHCO₃ (2×20 mL), brine (30 mL) and dried over Na₂SO₄. Filtration and solvent evaporation gave an oil, which was purified by flash chromatography (silica gel/hexanes:ethyl acetate 0-35%) to yield the desired 2-(pent-4-enyl)-isoindoline-1,3-dione as a solid (6.36 g, 0.029 mmol, 72.5% yield): MS m/e [M+H]⁺ Calcd 216.1. Found 216.1; NMR (250 MHz, DMSO-d₆) δ 7.79-7.95 (m, 4H), 5.70-5.91 (m, 1H), 4.90-5.11 (m, 2H), 3.58 (t, 2H), 1.98-2.10 (m, 2H), 1.59-1.78 (m, 2H).

### 2-(3-(Oxiran-2-yl)-propyl)-isoindoline-1,3-dione

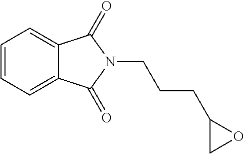

2-(Pent-4-enyl)-isoindoline-1,3-dione (6.36 g, 0.029 mmol) was submitted to Procedure 14 for epoxide formation to yield 2-(3-(oxiran-2-yl)-propyl-isoindoline-1,3-dione (5.8 g, 0.025 mmol, 86.2% yield): MS m/e [M+H]⁺ Calcd 232.1. Found 232.1; ¹H NMR (250 MHz, DMSO-d₆) δ 7.75-7.90 (m, 4H, Ar), 3.52 (t, 2H, CH₂), 2.87-2.96 (m, 1H, CH), 2.70 (t, 1H), 2.30-2.45 (m, 1H), 1.36-1.80 (m, 4H).

### N-Boc-3-hydroxypyrrolidine-3-carboxylic acid

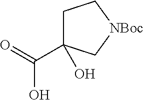

N-Boc-3-pyrrolidone (0.010 mmol) was submitted to Procedure 15 to yield the desired N-Boc-3-hydroxy-pyrrolidine-3-carboxylic acid.

### N-Boc-1-amino-but-3-ene

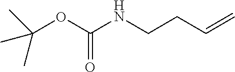

3-Buten-1-amine (4.93 g, 0.069 mol) was submitted to Procedure 13 for Boc protection to yield a crude, which was purified by flash chromatography (silica gel/hexanes:ethyl acetate 0-30%) to yield N-Boc-1-amino-but-3-ene (6.47 g, 0.038 mol, 55.1% yield).

### N-Boc-2-(oxiran-2-yl)-ethyl carbamate

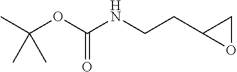

N-Boc-1-amino-but-3-ene (6.47 g, 0.038 mol) was submitted to Procedure 14 for epoxide formation to yield a crude, which was purified by flash chromatography (silica gel/hexanes:ethyl acetate 0-45%) to yield N-Boc-2-(oxiran-2-yl)-ethyl carbamate (6.0 g, 0.032 mol, 84.2% yield): ¹H NMR (250 MHz, DMSO-d₆) δ 2.98-3.09 (m, 2H), 2.83-2.92 (m, 1H), 2.65 (t, 1H), 2.42 (dd, 1H), 1.44-1.66 (m, 2H), 1.36 (s, 9H, (CH₃)₃).

### N-Boc-3-hydroxy-azetidin-3-carboxylic acid

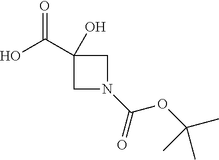

N-Boc-3-azetidinone (21.9 g, 0.128 mol) was submitted to Procedure to yield the desired N-Boc-3-hydroxy-azetidin-3-carboxylic acid (18.7 g, 0.086 mol, 67.0% yield): MS m/e [M+H]⁺ Calcd 218.1. Found 218.2.

### 3-Methylene-1-methylamino-cyclobutane

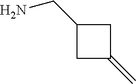

To a stirring solution of 3-methylene-1-cyano-cyclobutane (2.5 g, 0.026 mol) in THF (35 ml) at 0° C. was slowly added 2M LiAlH₄ (22 mL, 0.044 mmol) and the reaction was allowed to warm to room temperature. The reaction was then quenched by the addition of sat. aq. NH₄Cl (10 mL), and THF (10 mL). The organic layer was separated and concentrated to dryness to yield a residue, which was dissolved in ethyl acetate (100 mL). The organic layer was washed with 5% NaHCO₃ (2×20 mL), brine (20 mL), dried over Na₂SO₄, filtered and concentrated to yield the desired 3-methylene-1-methylamino-cyclobutane as an oil, which was carried through to the next step without further purification.

### 3-Methylene-1-N-Boc-methylamino-cyclobutane

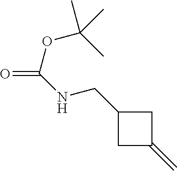

To a stirring solution of 3-methylene-1-methylamino-cyclobutane (2.52 g, 0.026 mol) in 1N NaOH (15 ml) and THF (15 mL), was added Boc₂O (6.7 g, 0.030 mol) and the reaction mixture was stirred overnight. THF was evaporated and the aqueous layer was extracted with ethyl acetate (2×40 mL). The combined organic layers were washed with 5% NaHCO₃ (2×20 mL) brine (20 mL), dried over Na₂SO₄, filtered and concentrated to dryness to yield a crude, which was purified by flash chromatography (silica gel/hexanes:ethyl acetate 0%-60%) to yield the desired 3-methylene-1-N-Boc-methylamino-cyclobutane (1.9 g, 0.0096 mol, 36.9% yield): ¹H NMR (250 MHz, DMSO-d₆) δ 6.88 (bs, 1H), 4.72 (s, 2H), 2.95-3.05 (m, 2H), 2.56-2.71 (m, 2H), 2.21-2.40 (m, 3H), 1.20 (s, 9H).

### N-Boc-1-oxaspiro[2.3]hexan-5-yl-methanamine

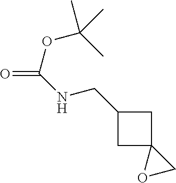

3-Methylene-1-N-Boc-methylamino-cyclobutane (1.9 g, 0.0096 mol) was submitted to Procedure 14 for epoxide formation to yield N-Boc-1-oxaspiro[2.3]hexan-5-yl-methanamine (1.34 g, 6.27 mol, 65.3% yield): ¹H NMR (250 MHz, DMSO-d₆) δ 2.99-3.10 (m, 2H), 2.60-2.66 (m, 2H), 1.99-2.47 (m, 5H), 1.40 (s, 9H).

### N-Fmoc-4-amino-butyraldehyde diethyl acetal

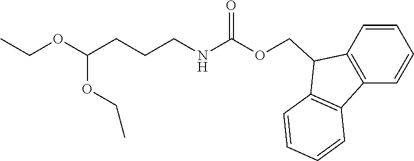

4-Amino-butyraldehyde diethyl acetal (8.0 g, 0.050 mol) was Fmoc protected following Procedure 16 to give the desired N-Fmoc-4-amino-butyraldehyde diethyl acetal (22.08 g, MS m/e [M+Na]⁺ Calcd 406.2. Found 406.1), which was carried through to the next step without further purification.

### N-Fmoc-4-amino-butyraldehyde

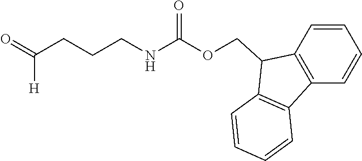

To a stirring solution of N-Fmoc-4-amino-butyraldehyde diethyl acetal (0.050 mmol) in 1,4-dioxane (100 mL) was added aq. HCl (100 ml, 1:1 v/v, H₂O: conc. HCl) and the reaction progress was monitored by MS. Upon completion, the organic solvent was removed by rotary evaporation, and the aqueous layer was extracted with ethyl acetate (2×200 mL). The combined organic layers were washed with 5% NaHCO₃ (2×75 mL), brine (75 mL), dried over Na₂SO₄, filtered and concentrated to dryness to yield the desired N-Fmoc-4-amino-butyraldehyde (15.35 g, 0.049 mol, 90.0% yield), which was carried through to the next step without further purification: MS m/e [M+Na]⁺ Calcd 332.1. Found 332.0.

### 3-Methylene-cyclobutane carboxylic acid

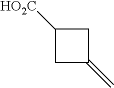

To a stirring solution of KOH (70.0 g, 1.25 mol) in EtOH/H₂O (500 mL, 1:1 v/v) was added 3-methylenecyclobutane carbonitrile (25.0 g, 0.26 mol) and the reaction mixture was refluxed for 6 h. The reaction progress was monitored by TLC and, upon completion, the mixture was cooled and acidified to pH 3-4 with HCl. The ethanol was evaporated, and the remaining aqueous layer was extracted with Et₂O (200 mL). The organic layer was washed with water (2×20 mL), brine (30 ml), dried over Na₂SO₄, filtered and concentrated to dryness to yield 3-methylene-cyclobutane carboxylic acid, which was carried through to the next step without further purification: ⁺H NMR (250 MHz, CDCl₃) δ 10.75 (bs, 1H), 4.80 (s, 2H), 2.85-3.26 (m, 5H).

### N-Boc-3-Methylene-cyclobutanamine

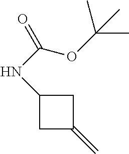

To a stirring solution of 3-methylene-cyclobutane carboxylic acid (1.0 g, 8.9 mmol) in THF (90 mL) was added NaN₃ (2.0 g, 31.1 mmol), followed by tetrabutyl ammonium bromide (0.48 g, 1.5 mmol) and Zn(OTf)₂ (0.1 g, 0.3 mmol), and the reaction mixture was heated to 40° C. Boc₂O (2.1 g, 9.8 mmol) was then added at once, and the reaction was heated at 45° C. overnight. The reaction was then cooled to 0° C. and was quenched with 10% aq. NaNO₂ (180 mL). The THF was evaporated and the aqueous layer was extracted with EtOAc (180 mL). The organic layer was washed with 5% aq. NaHCO₃ (2×20 mL), brine (30 ml), dried over Na₂SO₄, filtered and concentrated to dryness to yield a crude, which was purified by flash chromatography (silica gel/hexanes:ethyl acetate: 0-90%) to yield the desired N-Boc-3-methylene-cyclobutanamine (0.57 g, 3.1 mmol, 34.9% yield): ¹H NMR (250 MHz, CDCl₃) δ 4.83 (s, 2H), 4.79 (bs, 1H), 4.05-4.23 (m, 1H), 2.92-3.11 (m, 2H), 2.50-2.65 (m, 2H), 1.44 (s, 9H).

### N-Boc-1-oxaspiro[2.3]hexan-5-amine

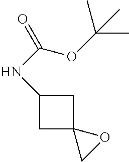

N-Boc-3-methylene-cyclobutanamine (1.65 g, 9.0 mmol) was submitted to Procedure 14 for epoxide formation to yield N-Boc-1-oxaspiro[2.3]hexan-5-amine (1.46 g, 7.33 mmol, 81.5% yield): ¹H NMR (250 MHz, CDCl₃) δ 4.79 (bs, 1H), 4.13-4.31 (m, 1H), 2.66-2.83 (m, 4H), 2.31-2.47 (m, 2H), 1.45 (s, 9H).

### N-Boc-2,2-dimethyl-3-amino-propionaldehyde

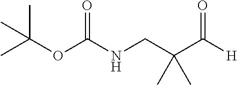

N-Boc-2,2-dimethyl propanol (0.415 g, 2.04 mmol) was submitted to Procedure 18 to yield N-Boc-2,2-dimethyl-3-amino-propionaldehyde (0.39 g, 1.94 mmol, 95.1% yield): ¹H NMR (250 MHz, CDCl₃) δ 9.42 (s, 1H), 4.80 (bs, 1H), 3.11 (d, 2H), 1.39 (s, 9H), 1.06 (s, 6H).

### N-Boc-3-amino-3-cyclopropyl propionaldehyde

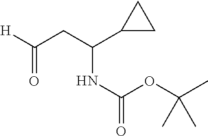

N-Boc-3-amino-propanol (0.130 g, 0.60 mmol) was submitted to Procedure 18 for oxidation to the corresponding N-Boc-3-amino-3-cyclopropyl propionaldehyde, which was carried through to the next step without further purification.

### 4(S)-tert-Butyldimethylsilyloxy-N-Boc-pyrrolidin-2(R)-carboxaldehyde

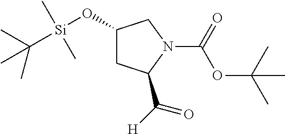

4(S)-tert-Butyldimethylsilyloxy-N-Boc-pyrrolidin-2(R)-methanol (0.50 g, 1.50 mmol) was submitted to Procedure 18 for oxidation to the corresponding 4(S)-tert-butyldimethylsilyloxy-N-Boc-pyrrolidin-2(R)-carboxaldehyde, which was carried through to the next step without further purification.

### 3-tert-Butyldimethylsilyloxy-propanal

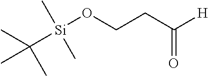

3-tert-Butyldimethylsilyloxy-propanol (0.50 g, 2.62 mmol) was submitted to Procedure 18 for oxidation to the corresponding 3-tert-butyldimethylsilyloxy-propanal, which was carried through to the next step without further purification.

### 2-Methyl-N-Boc-2-amino-propanal

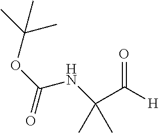

2-Methyl-N-Boc-2-amino-propanol (0.83 g, 4.38 mmol) was submitted to Procedure 18 for oxidation to the corresponding 2-methyl-N-Boc-2-amino-propanal (0.706 g, 3.77 mmol, 86.1% yield): ¹H NMR (250 MHz, CDCl₃) δ 9.40 (s, 1H), 1.57 (s, 1H), 1.41 (s, 9H), 1.30 (s, 6H).

### N-Boc-1-amino-cyclobutane carboxylic acid

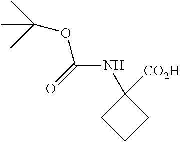

1-Amino-cyclobutane carboxylic acid ethyl ester (1.0 g, 6.28 mmol) was dissolved in 1N HCl (10 mL) and the reaction was heated to a reflux for 2 hours. The reaction mixture was then concentrated to dryness to yield a crude which was submitted to Procedure 13 for Boc protection to yield the desired N-Boc-1-Amino-cyclobutane carboxylic acid.

### N-Boc-1-amino-cyclobutyl-methanol

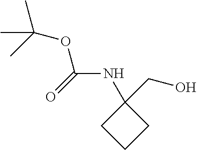

N-Boc-1-amino-cyclobutane carboxylic acid (6.28 mmol) was submitted to Procedure 19 for reduction to the corresponding N-Boc-1-Amino-cyclobutyl-methanol.

### N-Boc-1-amino-cyclobutane carboxaldehyde

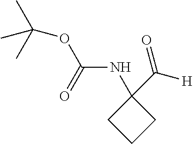

N-Boc-1-amino-cyclobutyl-methanol (0.25 g, 1.24 mmol) was submitted to Procedure 18 to yield the corresponding N-Boc-1-amino-cyclobutane carboxaldehyde (0.24 g, 1.20 mmol, 96.8% yield): ¹H NMR (250 MHz, CDCl₃) δ 9.0 (s, 1H), 4.91 (bs, 1H), 3.74 (bs, 2H), 1.71-2.20 (m, 4H), 1.42 (s, 9H).

### N-Boc-3-amino-cyclobutanone

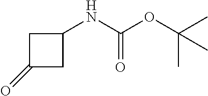

To a vigorously stirring solution of N-Boc-3-methylene-cyclobutanamine (9.8 g, 53.5 mmol) in DCM (160 mL) and H₂O (160 mL) was added K₂CO₃ (3 g, 21.7 mmol), followed by NaIO₄ (35 g, 163.5 mmol), tetrabutylammonium chloride (0.2 g, 0.72 mmol) and RuCl₃ (0.6 g, 7.6 mmol). During the course of the reaction, the organic solution turned dark brown, the catalyst turned black, while the upper aqueous layer turned white. The reaction was monitored by TLC, and upon completion, the reaction mixture was filtered through a pad of celite. The filtrates were transferred to a separatory funnel, and the aqueous layer was extracted with DCM (2×50 mL). The combined organic layers were washed with 5% NaHCO₃ (2×30 mL), brine (30 mL), dried over Na₂SO₄, filtered and evaporated to dryness to yield a crude, which was purified by flash chromatography (silica gel/hexanes:ethyl acetate 0-60%) to yield the desired N-Boc-3-amino-cyclobutanone (7.13 g, 38.53 mmol, 72% yield): NMR (250 MHz, CDCl₃) δ 4.88 (bs, 1H), 4.13-4.29 (m, 1H), 3.23-3.41 (m, 2H), 2.9-3.05 (m, 2H), 1.39 (s, 9H).

### N-Boc-1-hydroxy-3-amino-cyclobutyl-carboxylic acid

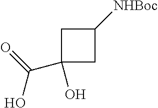

N-Boc-3-amino-cyclobutanone (7.13 g, 38.53 mmol) was submitted to Procedure 15 to yield the desired N-Boc-1-hydroxy-3-amino-cyclobutyl-carboxylic acid (MS m/e [M+H]⁺ Calcd 232.1. Found 232.2.

### N,N-diBoc-4(S)-amino-2(S)-methanol-pyrrolidine

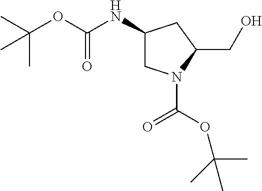

N,N-diBoc-4(S)-amino-pyrrolidine-2(S)-carboxylic acid (1.03 g, 3.12 mmol) was submitted to Procedure 19 to yield the corresponding N,N-diBoc-4(S)-amino-2(S)-methanol pyrrolidine (0.605 g, 1.91 mmol, 61.2% yield), which was carried through to the next step without further purification.

### N,N-diBoc-4(S)-amino-pyrrolidine-2(S)-carbaldehyde

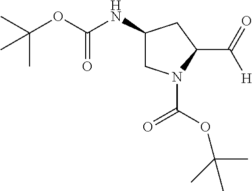

N,N-diBoc-4(S)-amino-2(S)-methanol pyrrolidine (0.486 g, 1.53 mmol) was submitted to Procedure 18 for oxidation to the corresponding N,N-diBoc-4(S)-amino-pyrrolidine-2(S)-carbaldehyde, which was carried through to the next step without further purification.

### N-Boc-1-aminomethyl-cyclopropyl-methanol

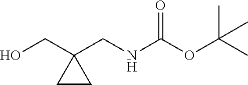

N-Boc-1-aminomethyl-cyclopropane carboxylic acid (1.0 g, 4.64 mmol) was submitted to Procedure 19 to yield the corresponding N-Boc-1-aminomethyl-cyclopropyl-methanol (0.99 g, MS m/e [M+H]⁺ Calcd 202.1. Found 202.1), which was carried through to the next step without further purification.

### N-Boc-1-aminomethyl-cyclopropane carboxaldehyde

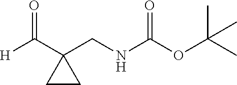

N-Boc-1-aminomethyl-cyclopropyl-methanol (0.87 g, 4.32 mmol) was submitted to Procedure 18 for oxidation to the corresponding N-Boc-1-aminomethyl-cyclopropane carboxaldehyde, which was carried through to the next step without further purification.

### N-Boc-1-amino-cyclopropyl-methanol

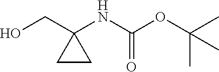

N-Boc-1-amino-cyclopropane carboxylic acid (0.25 g, 1.24 mmol) was submitted to Procedure 19 to yield the corresponding N-Boc-1-amino-cyclopropyl-methanol (0.051 g, 0.27 mmol, 21.8% yield), which was carried through to the next step without further purification.

### N-Boc-1-amino-cyclopropane carboxaldehyde

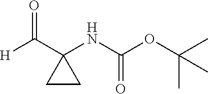

N-Boc-1-amino-cyclopropyl-methanol (0.051 g, 0.27 mmol) was submitted to Procedure 18 for oxidation to the corresponding N-Boc-1-amino-cyclopropane carboxaldehyde, which was carried through to the next step without further purification.

### N-Boc-1(R)-amino-2(S)-tert-butyldimethylsilyloxy-cyclopentane-4(S)-carboxylic acid

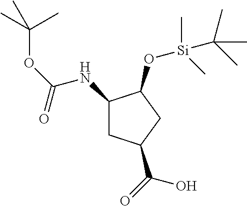

To a stirring solution of N-Boc-1(R)-amino-2(S)-hydroxy-cyclopentane-4(S)-carboxylic acid methyl ester (0.622 g, 2.40 mmol) in DCM (1.9 mL) was added imidazole (0.164 g, 2.41 mmol), DMAP (0.047 g, 0.35 mmol) and TBSCl (0.363 g, 2.40 mmol) and the reaction was stirred at room temperature for 18 hours, followed by heating at 40° C. for 1 hour. The reaction mixture was cooled to room temperature, and was quenched with H₂O (3 mL). The organic layer was separated and was concentrated to dryness to yield a residue, which was dissolved in isopropanol (6 mL) and 1M NaOH (2.9 mL), and the reaction was heated at 60° C. for 1 hour. The reaction was cooled to 0° C. and slowly acidified to pH 3 with 1M HCl (3 mL). After adding chloroform (18 mL), the organic layer was separated, dried over Na₂SO₄, and concentrated to dryness to yield the desired acid (0.75 g, 2.09 mmol, 87.1% yield).

### N-Boc-1(R)-amino-2(S)-tert-butyldimethylsilyloxy-4(S)-hydroxymethyl-cyclopentane

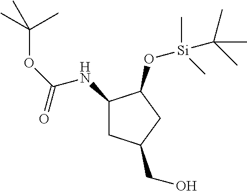

N-Boc-1(R)-amino-2(S)-tert-butyldimethylsilyloxy-cyclopentane-4(S)-carboxylic acid (0.53 g, 1.47 mmol) was submitted to Procedure 19 for reduction to the corresponding N-Boc-1(R)-amino-2(S)-tert-butyldimethylsilyloxy-4(S)-hydroxymethyl-cyclopentane (0.44 g, 1.27 mmol, 86.4% yield): ¹H NMR (250 MHz, CDCl₃) δ 4.69-4.79 (m, 1H), 4.08-4.13 (m, 1H), 3.88 (bs, 1H), 3.52-3.61 (m, 2H), 2.16-2.30 (m, 2H), 1.96-2.14 (m, 2H), 1.48-1.53 (m, 2H), 1.47 (s, 9H), 0.91 (s, 9H), 0.09 (s, 6H).

### N-Boc-1(R)-amino-2(S)-tert-butyldimethylsilyloxy-cyclopentane-4(S)-carboxaldehyde

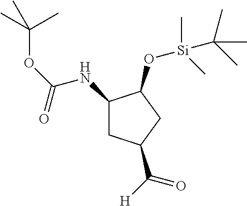

N-Boc-1(R)-amino-2(S)-tert-butyldimethylsilyloxy-4(S)-hydroxymethyl-cyclopentane (0.44 g, 1.27 mmol) was submitted to Procedure 18 for oxidation to the corresponding N-Boc-1(R)-amino-2(S)-tert-butyldimethylsilyloxy-cyclopentane-4(S)-carboxaldehyde (0.42 g, 1.22 mmol, 96.1% yield).

### tert-Butyl-2-(N-Boc-3-hydroxy-azetidin-3-yl)acetate

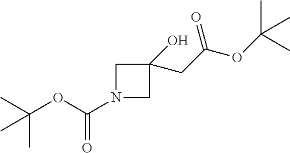

To a stirring solution of N-Boc-3-azetidinone (0.45 g, 2.64 mmol) in THF (5 mL) was slowly added a 0.5 M solution of 2-tert-butoxy-2-oxoethyl-zinc chloride in Et₂O (10 mL, 5.0 mmol), and the reaction mixture was stirred for 5 h. The reaction was then quenched with sat. aq. NH₄Cl (10 mL), and the aqueous layer was separated and extracted with ethyl acetate (2×30 mL). The combined organic layers were washed with 5% aq. NaHCO₃ (2×10 mL), brine (15 mL), dried over Na₂SO₄, filtered and concentrated to dryness to yield tert-butyl-2-(N-Boc-3-hydroxy-azetidin-3-yl)-acetate (MS m/e [M+H]⁺ Calcd 288.2. Found 287.7).

### 2-(N-Boc-3-hydroxy-azetidin-3-yl)-acetic acid

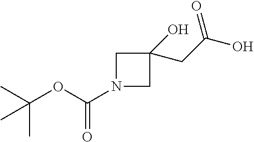

To a stirring solution of tert-butyl-2-(N-Boc-3-hydroxy-azetidin-3-yl)-acetate (0.86 g, 2.99 mmol) in dioxane (18 mL) was added 3M HCl (5 mL), and the mixture was heated at 70° C. for 1 h. The reaction mixture was then cooled to 0° C. and it was basified with 2 M NaOH (8 mL), followed by addition of BOC₂O (1.0 g, 4.6 mmol). The reaction mixture was allowed to warm to room temperature for 2 h, and was then concentrated to half its total volume on the rotary evaporator. Isopropanol (3 mL) and chloroform (12 mL) were then added and the mixture was cooled to 0° C. and slowly acidified to pH 3 with 1M HCl. The organic layer was then separated, dried over Na₂SO₄, and concentrated to dryness to yield 2-(N-Boc-3-hydroxy-azetidin-3-yl)-acetic acid (0.65 g, 2.81 mmol, 94.0% yield).

### N-Boc-3-(2-hydroxy-ethyl)-azetidin-3-ol

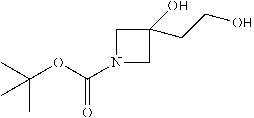

2-(N-Boc-3-hydroxy-azetidin-3-yl)-acetic acid (0.44 g, 1.90 mmol) was submitted to Procedure 19 for reduction to yield the corresponding N-Boc-3-(2-hydroxy-ethyl)-azetidin-3-ol (0.29 g, 1.33 mmol, 70.0% yield).

### 2-(N-Boc-3-hydroxy-azetidin-3-yl)-acetaldehyde

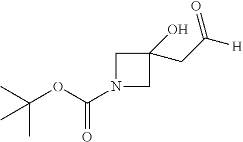

N-Boc-3-(2-hydroxy-ethyl)-azetidin-3-ol (0.29 g, 1.33 mmol) was submitted to Procedure 18 for oxidation to the corresponding 2-(N-Boc-3-hydroxy-azetidin-3-yl)-acetaldehyde, which was carried through to the next step without further purification.

### N-Boc-3-hydroxymethyl-azetidine

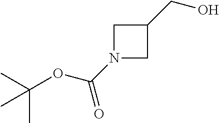

N-Boc-azetidine-3-carboxylic acid (1.94 g, 9.64 mmol) was submitted to Procedure 19 for reduction to the corresponding N-Boc-3-hydroxymethyl-azetidine, which was carried through to the next step without further purification.

### N-Boc-azetidine-3-carboxaldehyde

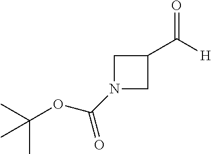

N-Boc-3-hydroxymethyl-azetidine (9.64 mmol) was submitted to Procedure 18 for oxidation to the desired N-Boc-azetidine-3-carboxaldehyde, which was carried through to the next step without further purification.

### 2-(N-Boc-azetidin-3-yl)-2-hydroxy-acetic acid

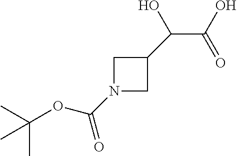

N-Boc-azetidine-3-carboxaldehyde (1.60 g, 8.64 mmol) was submitted to Procedure 15 to yield the desired 2-(N-Boc-azetidin-3-yl)-2-hydroxy-acetic acid (MS m/e [M+H]⁺ Calcd 232.1. Found 231.8).

### N,N′-bis-Cbz-2(S)-hydroxy-4-guanidino-butyric acid

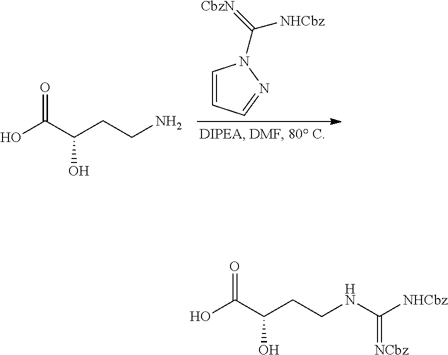

To a stirring solution of 2(S)-hydroxy-4-amino-butyric acid (0.059 g, 0.50 mmol) in DMF (2 ml) was added N,N′-bis(benzyloxycarbonyl)-1H-pyrazole-1-carboxamidine (0.26 g, 0.70 mmol) followed by DIPEA (0.87 mL, 4.99 mmol) and the reaction was heated to 80° C. and stirred overnight. The crude mixture was purified on a 2-inch reverse-phase HPLC column to yield N,N′-bis-Cbz-2(S)-hydroxy-4-guanidino-butyric acid: MS: m/z (M+H)⁺ Calcd. 430.15. Found 430.1.

### Benzyl-2-(benzoyloxyamino)ethyl carbamate

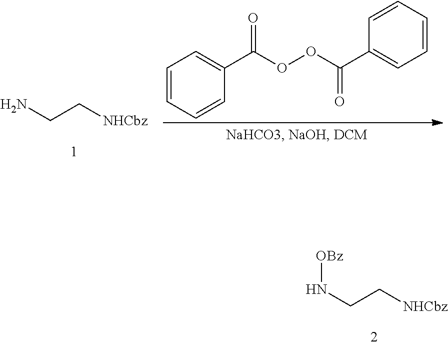

To a solution of benzyl-N-(2-aminoethyl)carbamate chloride salt (1, 540 mg, 2.34 mmol) in sat. aq. NaHCO₃ (45 mL) was added 1M NaOH (15 mL) and the reaction was stirred vigorously. DCM (30 mL) was added, followed by benzoylperoxide (1.13 g, 4.68 mmol) and the reaction was stirred overnight. The organic layer was separated and washed with brine, dried over MgSO₄, filtered and concentrated to a crude, which was purified on a 1-inch reverse-phase HPLC column to yield benzyl-2-(benzoyloxyamino)ethyl carbamate (2, 252 mg, 0.80 mmol, 34.2%): MS: m/z (M+H)⁺ Calc. 315.13, obs. 315.0.

### Succinimidyl benzoyloxy(2-Cbz-aminoethyl)carbamate

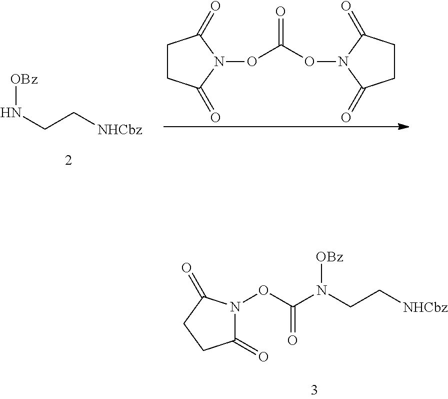

To a stirring solution of disuccinimidyl carbonate (525 mg, 2.05 mmol) in CH₃CN (16 mL) was added benzyl-2-(benzoyloxyamino)ethyl carbamate (2, 252 mg, 0.80 mmol) as a solution in CH₃CN (12 mL) over 4 hours, and the reaction was stirred overnight. Additional disuccinimidyl carbonate (251 mg, 0.98 mmol) was added and the reaction was heated at 60° C. overnight. Solvent removal gave a crude, which was purified on a 2-inch reverse-phase HPLC column to yield succinimidyl benzoyloxy(2-Cbz-aminoethyl)carbamate (3, 81 mg, 0.18 mmol, 22.5% yield).

### Synthesis of (2R,3R)-4-azido-2-benzyloxy-3-fluorobutanoic acid (5)

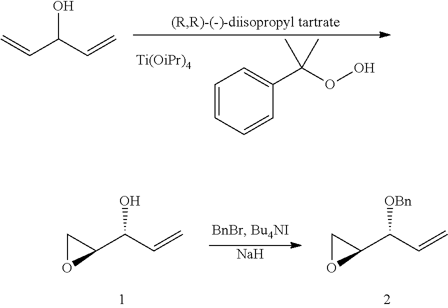

Molecular sieves (4 Å, 4 g) were added to a round bottom flask, and were activated by heating with a Bunsen burner under high vacuum. DCM (100 mL) was then added and the flask was cooled to −35° C. with a cryocooler. Titanium tetraisopropoxide (1.75 mL, 5.95 mmol) and (R,R)-(−)-diisopropyl tartrate (1.65 mL, 7.75 mmol) were added and the reaction was stirred for 30 min. Penta-1,4-dienol (5 g, 59.4 mmol) and excess cumene hydroperoxide (80%, 17.5 mL) were added in small portions, and stirring was continued at −35° C. for 48 hr. The reaction was quenched by addition of sat. aq. Na₂SO₄ (5 mL) immediately followed by Et₂O (50 mL) and the reaction was stirred for 2 hr with warming to rt. The reaction mixture was filtered through Celite, and washed with Et₂O, Solvent removal under vacuum without heating resulted in approximately 30 mL of a yellow solution. Excess cumene alcohol and hydroperoxide were removed by flash chromatography (silica gel, 40% Et₂O/hex). Finally solvent removal under vacuum without heating yielded a mixture of (2S,3R)-1,2-epoxy-4-penten-3-ol (1) (Rf=0.47, 1:1 EtOAc/hex) and diisopropyl tartrate (Rf=0.6), which was used in the next step without further purification.

To a stirring solution of epoxide (1) in THF (100 mL) under an argon atmosphere was added tetrabutylammonium iodide (2.2 g, 5.96 mmol), followed by benzyl bromide (8.6 mL, 71.9 mmol) and the reaction was cooled to −15° C. Sodium hydride (60% in mineral oil, 2.65 g, 66.1 mmol) was added in small portions and the reaction was stirred overnight with warming to rt. The reaction was quenched with MeOH, filtered through Celite, and washed with Et₂O. Solvent removal gave an oily residue which was purified by flash chromatography (silica gel, 5→10% Et₂O/hex) to yield (2S,3R)-1,2-epoxy-3-benzyloxy-4-pentene (2) as a clear non-volatile liquid (5.3 g, 47.6% yield): Rf=0.69 (1:4 EtOAc/hex); [α]_{D}=−36.7° (c 1.52, CHCl₃); HRMS (ESI) (M+H)⁺ Calc. for C₁₂H₁₄O₂ 191.1067, obs. 191.1064; ¹H NMR (CDCl₃, 300 MHz) δ 7.38-7.33 (m, 5H), 5.92-5.78 (m, 1H), 5.41-5.39 (m, 1H), 5.37-5.33 (m, 1H), 4.66 (d, J=11.95 Hz, 1H), 4.49 (d, J=11.96 Hz, 1H), 3.83 (dd, J=7.34, 4.20 Hz, 1H), 3.10 (dt, J=4.07, 4.06, 2.70 Hz, 1H), 2.79 (dd, J=5.21, 4.00 Hz, 1H), 2.70 (dd, J=5.23, 2.64 Hz, 1H). ¹³C NMR (CDCl₃, 100 MHz) δ 138.32, 134.67, 128.56 (2C), 127.87 (2C), 127.82, 119.73, 79.54, 70.83, 53.41, 45.00.

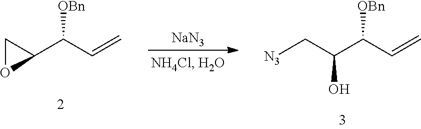

NaN₃ (3.38 g, 52 mmol) and NH₄Cl (2.78 g, 52 mmol) in H₂O (10 mL) were heated until a clear solution was obtained. This solution was then added dropwise to a solution of (2S,3R)-1,2-epoxy-3-benzyloxy-4-pentene (2) (3.3 g, 17.4 mmol) in MeOH (200 mL) and the reaction mixture was stirred for 4 days. The organic solvent was removed under vacuum, and the aqueous layer was extracted with DCM (3×). The combined organic layers were dried over Na₂SO₄, filtered and reduced under vacuum to yield a crude, which was purified by flash chromatography (silica gel, 10→20% Et₂O/hex) to yield (2S,3R)-1-azido-3-benzyloxy-4-penten-2-ol (3) (2.66 g, 66% yield) as a non-volatile clear liquid: Rf=4.8 (1:4 EtOAc/hex); HRMS (ESI) (M+Na)⁺ Calc. for C₁₂H₁₅N₃O₂ 256.1056, obs. 256.1057; [α]_{D}=−46.3° (c 1.50, CHCl₃); ¹H NMR (CDCl₃, 300 MHz) δ 7.42-7.28 (m, 5H), 5.91-5.76 (m, 1H), 5.46 (dd, J=17.16, 1.42 Hz, 1H), 5.42 (dd, J=24.00, 1.37 Hz, 1H), 4.65 (d, J=11.67 Hz, 1H), 4.39 (d, J=11.67 Hz, 1H), 3.88-3.80 (m, 2H), 3.44-3.40 (m, 2H), 2.22 (d, J=3.60 Hz, 1H); ¹³C NMR (CDCl₃, 100 MHz) δ 137.88, 134.60, 128.66 (2C), 128.08 (2C), 128.05, 121.40, 81.39, 72.61, 70.70, 53.0; FTIR (NaCl): 3435, 2870, 2102, 1642, 1454, 1070 cm⁻¹.

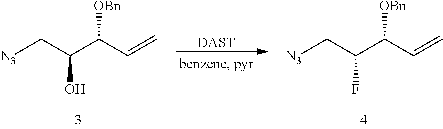

To a stirring solution of DAST (900 μL, 6.87 mmol) in benzene (3.2 mL) and pyridine (400 μL) in a plastic container at −10° C. was added (2S,3R)-1-azido-3-benzyloxy-4-penten-2-ol (3) (750 mg, 3.21 mmol) in small portions, and the reaction was stirred at this temperature for 48 hr followed by 6 hr at rt. The reaction mixture was slowly added to sat. aq. NaHCO₃ (20 mL) at 0° C. and was stirred for 10 min. The resulting aqueous mixture was extracted with DCM (3×) and the combined organic layers were washed with 2N HCl, dried over MgSO₄, filtered and reduced under vacuum to yield a crude, which was purified by flash chromatography (silica gel, 1% Et₂O/hex) to yield (3R,4R)-5-azido-4-fluoro-3-benzyloxy-pent-1-ene (4) (128 mg, 16.9% yield) as a nonvolatile clear liquid: Rf=0.63 (1:9 EtOAC/Hex); [α]_{D}=−11.9° (c 1.50, CHCl₃); ¹H NMR (CDCl₃, 400 MHz) δ 7.44-7.29 (m, 5H), 4.63 (dddd, J=47.64, 7.07, 4.99, 3.32 Hz, 1H), 5.49-5.42 (m, 2H), 4.70 (d, J=11.95 Hz, 1H), 4.57 (ddd, J=7.07, 4.99, 3.32 Hz, 1H), 4.44 (d, J=11.90 Hz, 1H), 4.03 (ddd, J=16.87, 7.57, 5.04 Hz, 1H), 3.64-3.52 (m, 1H), 3.45 (ddd, J=27.45, 13.63, 3.27 Hz, 1H). ¹⁹F NMR (CDCl₃, 282 MHz)-196.66 (dddd, J=47.27, 27.08, 19.84, 16.89 Hz); ¹³C NMR (CDCl₃, 100 MHz) δ 137.80, 133.09 (d, J=5.30 Hz), 128.70 (2C), 128.09 (3C), 121.04, 93.33 (d, J=181.54 Hz), 79.08 (d, J=20.39 Hz), 70.92, 51.46 (d, J=22.25 Hz). FTIR (NaCl): 2930, 2104, 1643, 1454, 1281, 1115, 1069 cm⁻¹.

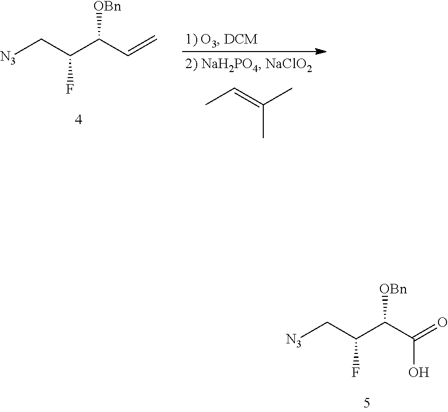

(3R,4R)-5-azido-4-fluoro-3-benzyloxy-pent-1-ene (4) (128 mg, 0.543 mmol) was submitted to Procedure 21, followed by recrystallization from hot hexanes (2×) to yield (2R,3R)-4-azido-2-benzyloxy-3-fluorobutanoic acid (5) (120 mg, 90%): [α]_{D}=−56.9° (c 0.68, CHCl₃); HRMS (ESI negative mode) (M−H) Calc. for C₁₁H₁₂FN₃O₃ 252.0790, obs. 252.0782; ¹H NMR (CDCl₃, 400 MHz). δ 10.55 (s, 1H), 7.46-7.34 (m, 5H), 4.98 (dddd, J=46.40, 7.57, 4.91, 2.92 Hz, 1H), 4.94 (d, J=11.47 Hz, 1H), 4.55 (d, J=11.51 Hz, 1H), 4.17 (dd, J=27.26, 2.86 Hz, 1H), 3.77 (dt, J=13.89, 13.66, 7.27 Hz, 1H), 3.42 (ddd, J=24.28, 13.20, 4.92 Hz, 1H); ¹⁹F NMR (CDCl₃, 376 MHz) δ −198.36 (dddd, J=46.28, 27.22, 24.46, 14.15 Hz); ¹³C NMR (CDCl₃, 100 MHz) δ 174.63 (d, J=4.21 Hz), 136.37, 129.15 (2C), 129.07, 128.98 (2C), 91.53 (d, J=182.59 Hz), 76.40 (d, J=19.90 Hz), 73.96 (s), 50.87 (d, J=25.13 Hz); FTIR (NaCl): 3151, 2098, 1753, 1407, 1283, 1112 cm⁻¹.

### Synthesis of ent-5

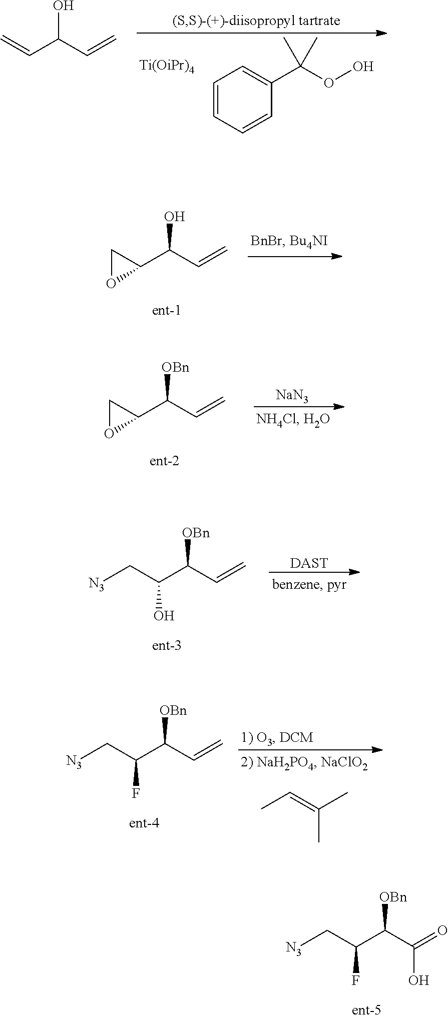

Starting from penta-1,4-dienol (5 g, 59.4 mmol) and using (S,S)-(+)-diisopropyl tartrate under the same reaction conditions as described above the enantiomer ent-2 was obtained (4.9 g, 43% yield): [α]_{D}=+35.7° (c 1.76, CHCl₃). (2R, 3S)-1,2-Epoxy-3-benzyloxy-4-pentene (ent-2, 3.9 g, 20.5 mmol) was submitted to the same reaction conditions described above to yield the enantiomer (2R,3S)-1-azido-3-benzyloxy-4-penten-2-ol (ent-3, 2.75 g, 57% yield): [α]_{D}=+ 47.3° (c 1.30, CHCl₃). (2R,3S)-1-Azido-3-benzyloxy-4-penten-2-ol (ent-3) (500 mg, 2.14 mmol) was submitted to the same reactions as described above to yield the enantiomer (3S,4S)-5-azido-4-fluoro-3-benzyloxy-pent-1-ene (ent-4, 75.5 mg, 0.32 mmol, 15% yield, [α]_{D}=+10.7°, c 1.50, CHCl₃), which was submitted to the same reaction conditions as described above to yield ent-5 (59 mg, 73% yield): [α]_{D}=+58.6° (c 0.73, CHCl₃).

### Synthesis of (R)-4-Azido-3,3-difluoro-2-benzyloxy-butanoic acid (3)

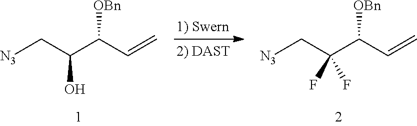

To a stirring solution of DMSO (690 μL, 9.65 mmol) in DCM (25 mL) at −78° C. was added oxalyl chloride (3.21 mL of a 2.0M solution in DCM, 6.43 mmol) and the reaction was stirred for 1 hr. A solution of (2S,3R)-1-azido-3-benzyloxy-4-penten-2-ol (1) (750 mg, 3.21 mmol) in DCM (1 mL) was added dropwise and the reaction mixture was stirred for 1 hr at −78° C. N-Methyl morpholine (1.41 mL, 12.9 mmol) was added dropwise, and the reaction was stirred at −15° C. for 2 hr. The reaction was quenched with phosphate buffer (0.1M, pH 6.0) and the aqueous layer was separated. The organic layer was washed with the phosphate buffer (3×), dried over Na₂SO₄, filtered and reduced under vacuum to give a brown residue. The residue was dissolved in Et₂O, dried over MgSO₄, filtered through a cotton plug, and reduced under vacuum to yield the crude ketone, which was dissolved in DCM (1 mL) and was added to a stirring solution of DAST (2 mL, 15.3 mmol) in DCM (3 mL) in a plastic vial at −25° C. The reaction was allowed to slowly warm to rt and was stirred for 48 hr. The reaction mixture was then slowly poured into stirring sat. aq. NaHCO₃ (20 mL) at 0° C., and was stirred for 10 min. The resulting aqueous mixture was extracted with DCM (3×), and the combined organic layers were dried over Na₂SO₄, filtered and reduced under vacuum to yield a crude, which was purified by flash chromatography (silica gel, 1% Et₂O/hex) followed by preparative TLC purification (silica gel, 0.5 mm, 5% Et₂O/hex) to yield (R)-5-azido-4,4-difluoro-3-benzyloxy-pent-1-ene (2, 193 mg, 0.76 mmol, 24% yield), as a non-volatile clear liquid: Rf=0.72 (1:4 EtOAc/hex); [α]_{D}=−23.8° (c 1.52, CHCl₃); ¹H NMR (CDCl₃, 300 MHz) δ 7.44-7.31 (m, 5H), 5.89 (dddd, J=16.88, 10.61, 7.11, 0.62 Hz, 1H), 5.59-5.56 (m, 1H), 5.53 (d, J=10.74 Hz, 1H), 4.71 (d, J=11.67 Hz, 1H), 4.50 (d, J=11.66 Hz, 1H), 4.14 (td, J=14.25, 7.13, 7.13 Hz, 1H), 3.64 (tq, J=13.67, 13.67, 13.67, 11.19, 11.19 Hz, 2H); ¹⁹F NMR (CDCl₃, 282 MHz) δ −116.63 (dtd, J=257.62, 13.91, 13.90, 8.72 Hz), −111.27 (dtd, J=257.59, 16.18, 16.16, 7.04 Hz); ¹³C NMR (CDCl₃, 75 MHz) δ 137.14, 130.33 (t, J=3.06, 3.06 Hz), 128.71 (2C), 128.27, 128.20 (2C), 122.78, 120.69 (dd, J=249.89, 246.83 Hz), 78.87 (dd, J=30.35, 25.35 Hz), 71.48 (d, J=0.48 Hz), 51.47 (dd, J=30.26, 25.92 Hz); FTIR (NaCl): 2928, 2108, 1455, 1292, 1091 cm⁻¹.

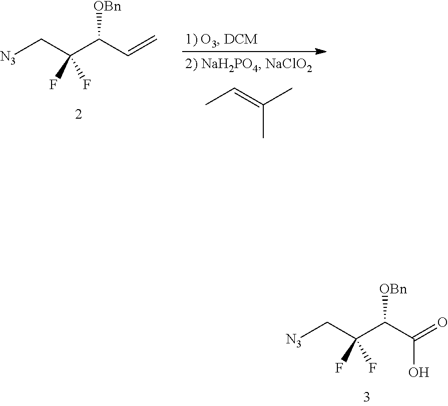

(R)-5-Azido-4,4-difluoro-3-benzyloxy-pent-1-ene (2, 193 mg, 0.76 mmol) was submitted to Procedure 21, followed by washing with cold hexanes (3×) at −20° C. to yield (3) (139 mg, 67.6% yield): [α]_{D}=−32.4° (c 0.80, CHCl₃); HRMS (ESI negative mode) (M−H) for C₁₁H₁₁F₂N₃O₃ 270.0696, obs. 270.06924; ¹H NMR (CDCl₃, 400 MHz) δ 7.46-7.32 (m, 5H), 6.48 (s, 1H), 4.84 (d, J=11.30 Hz, 1H), 4.67 (d, J=11.30 Hz, 1H), 4.37 (dd, J=12.23, 9.78 Hz, 1H), 3.75 (dd, J=14.67, 12.35 Hz, 2H); ¹⁹F NMR (CDCl₃, 376 MHz) δ −112.61 (qd, J=260.95, 12.30, 12.29, 12.29 Hz), −109.68 (dtd, J=260.79, 14.75, 14.68, 9.94 Hz); ¹³C NMR (CDCl₃, 100 MHz) δ 170.84, 135.48, 129.01, 128.94 (2C), 128.78 (2C), 119.59 (t, J=251.58, 251.58 Hz), 76.56 (dd, J=29.86, 27.24 Hz), 74.34, 51.58 (dd, J=28.94, 26.76 Hz). FTIR (NaCl): 3337, 2929, 2112, 1738, 1455, 1292, 1210, 1119 cm⁻¹.

### Synthesis of ent-3

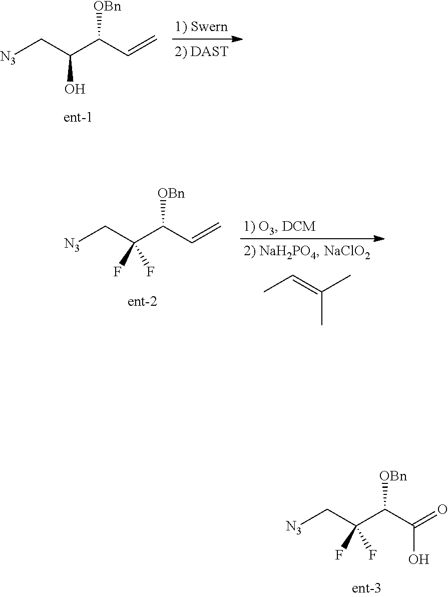

(2R,3S)-1-Azido-3-benzyloxy-4-penten-2-ol (ent-1, 500 mg, 2.14 mmol) was submitted to the same reaction conditions described above to yield (S)-5-azido-4,4-difluoro-3-benzyloxy-pent-1-ene (ent-2, 114 mg, 21% yield, [α]_{D}=+27.9° (c 3.14, CHCl₃)). Ent-2 (75.5 mg, 0.32 mmol) was submitted to Procedure 21 to yield (S)-4-azido-2-benzyloxy-3,3-difluorobutanoic acid (ent-3, 34.8 mg, 43% yield, [α]_{D}=+36.4° (c 0.80, CHCl₃).

### Synthesis of (2S,3S)-4-azido-2,3-bis-benzyloxybutanoic acid (3)

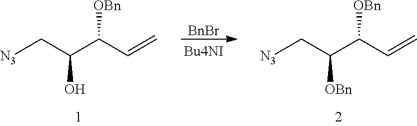

To a stirring solution of (2S,3R)-1-azido-3-benzyloxy-4-penten-2-ol (1) (250 μL, 1.07 mmol) in THF (50 mL) under argon was added tetrabutylammonium iodide (42 mg, 0.11 mmol) followed by benzyl bromide (155 μL, 1.27 mmol) and the reaction was cooled to 0° C. Sodium hydride (60% in mineral oil, 47 mg, 1.18 mmol) was added in small portions and the reaction was stirred overnight with warming to rt. The reaction was quenched with MeOH, filtered through Celite, and washed with Et₂O. The organic solvent was removed under vacuum to give an oily residue, which was purified by flash chromatography (silica gel, 2% Et₂O/hex) to yield (3R,4S)-5-azido-3,4-bisbenzyloxy-pent-1-ene (2, 237 mg, 65% yield) as a clear non-volatile liquid: Rf=0.62 (1:4 EtOAc/hex); [α]_{D}=−6.1° (c 1.50, CHCl₃); ¹H NMR (CDCl₃, 300 MHz) δ 7.35-7.24 (m, 10H), 5.81 (ddd, J=17.15, 10.58, 7.45 Hz, 1H), 5.37 (ddd, J=5.70, 1.65, 0.86 Hz, 1H), 5.33 (ddd, J=12.07, 1.44, 0.81 Hz, 1H), 4.63 (s, 2H), 4.61 (d, J=11.87 Hz, 1H), 4.35 (d, J=11.78 Hz, 1H), 3.90 (tdd, J=7.37, 5.65, 0.79, 0.79 Hz, 1H), 3.60 (ddd, J=6.39, 5.69, 3.64 Hz, 1H), 3.43 (dd, J=12.93, 6.42 Hz, 1H), 3.35 (dd, J=12.93, 3.60 Hz, 1H); ¹³C NMR (CDCl₃, 75 MHz) δ 138.25, 138.01, 135.43, 128.60 (4C), 128.29 (2C), 128.02, 127.99 (2C), 127.87, 119.97, 80.76, 80.23, 73.33, 70.79, 51.69; FTIR (NaCl): 2867, 2100, 1606, 1454, 1286, 1095, 1073.

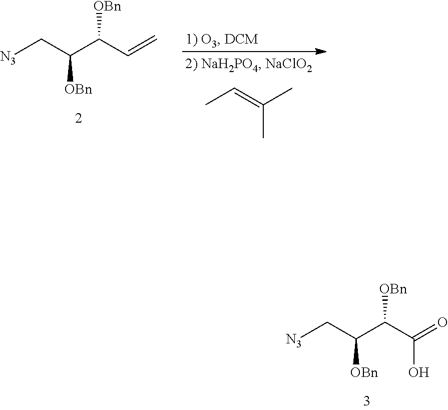

(3R,4S)-5-azido-3,4-bis-benzyloxy-pent-1-ene (2, 237 mg, 0.69 mmol) was submitted to Procedure 21 to yield (2S,3S)-4-azido-2,3-bis-benzyloxybutanoic acid (3, 187.7 mg, 75% yield): [α]_{D}=−15.1° (c 1.05, CHCl₃); HRMS (ESI negative mode) (M−H) Calc. for C₁₈H₁₉N₃O₄ 340.1303, obs. 340.1296; ¹H NMR (CDCl₃, 300 MHz) δ 7.24 (s, 1H), 7.38-7.33 (m, 10H), 4.79 (d, J=11.61 Hz, 1H), 4.66 (s, 2H), 4.56 (d, J=11.61 Hz, 1H), 4.20 (d, J=4.24 Hz, 1H), 3.98 (td, J=6.56, 4.30, 4.30 Hz, 1H), 3.58 (dd, J=13.04, 6.62 Hz, 1H), 3.42 (dd, J=13.04, 4.31 Hz, 1H); ¹³C NMR (CDCl₃, 75 MHz) δ 175.57, 137.92, 137.34, 129.44 (2C), 129.36 (2C), 129.15, 129.04 (2C), 128.98 (2C), 128.94, 79.71, 77.651, 74.04, 73.89, 51.65; FTIR (NaCl): 3000, 2918, 2103, 1722, 1455, 1284, 1110 cm⁻¹.

### Synthesis of ent-3

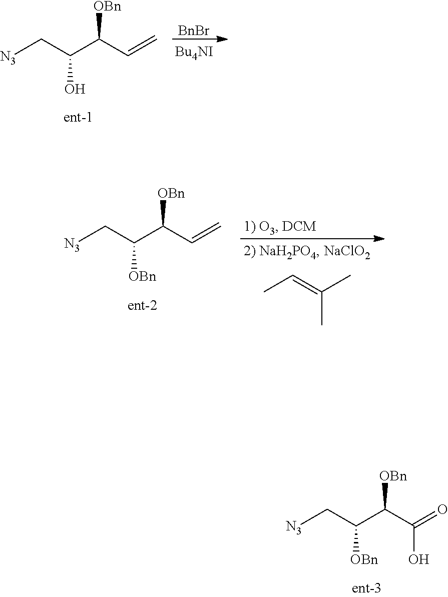

(2R,3S)-1-azido-3-benzyloxy-4-penten-2-ol (ent-1, 250 mg, 1.07 mmol) was submitted to the same reaction conditions as described above to yield (3S,4R)-5-azido-3,4-bis-benzyloxy-pent-1-ene (ent-2, 322 mg, 59% yield): [α]_{D}=+7.9° (c 1.50, CHCl₃). Ent-2 (178 mg, 0.55 mmol) was submitted to Procedure 21 to yield ent-3 (144 mg, 77% yield): [α]_{D}=+15.2° (c 0.81, CHCl₃).

### Synthesis of Compound 9

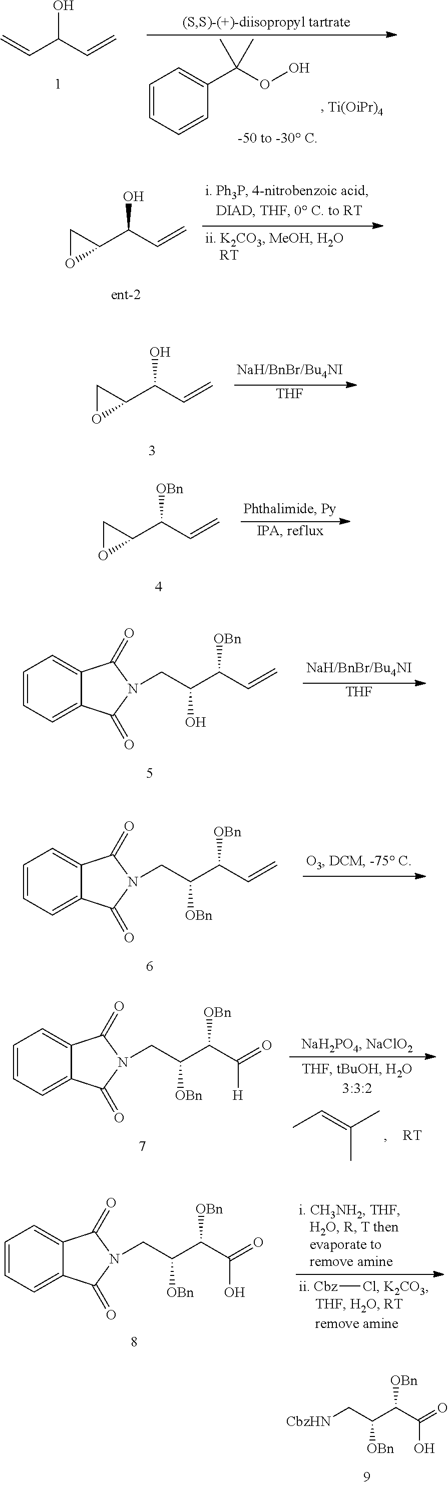

### Synthesis of Epoxy Alcohol Ent-2

A 3-neck, 5 liter round bottomed flask equipped with an overhead mechanical stirrer, a thermocouple probe and a nitrogen inlet/outlet was charged with powdered, freshly activated molecular sieves (4 Å, 84 g, 0.8 wt. equiv), followed by anhydrous dichloromethane (2.1 L, 20 vol). The resulting suspension was cooled to approximately −42° C. using an acetonitrile/CO₂ bath, then titanium tetraisopropoxide (37 mL, 0.125 mol, 10 mol %) was charged into the batch, followed by (S,S)-(+)-diisopropyl tartrate (35 mL, 0.166 mol, 13.3 mol %). The reaction mixture was stirred for 30 minutes, then divinyl alcohol 1 (105 g, 1.25 mol, 1.0 equiv) was added over 3 minutes using an addition funnel (minor exotherm, 2° C.). Cumene hydroperoxide (370 mL, 80% titer, 1.99 mol, 1.59 equiv) was then added to the batch over 5 minutes using an addition funnel (10° C. exotherm). The reaction was allowed to proceed for 18 hours, holding the temperature between −45 and −30° C. When complete as determined by TLC analysis (R_{f} 0.42 for divinyl alcohol, and 0.18 for epoxy alcohol, 50% MTBE in Heptanes), the reaction was quenched with saturated aqueous sodium sulfate (105 mL, 1 vol), diluted with MTBE (1.05 L, 10 vol) and the batch allowed to warm to ambient temperature, with vigorous stirring. Diatomaceous earth, Celite® (105 g, 1 wt. equiv) was added to the batch, which was then filtered through a pad of Celite®. The filter cake was washed with MTBE (0.5 L) and the filtrate concentrated in vacuo on a rotary evaporator (with water bath held at 10-20° C.) to afford a yellow/brownish oil. A portion of the crude product [311 g] was subjected to silica plug (1 kg silica gel) using 0-60% MTBE/petroleum ether. The fractions containing the product were collected and concentrated to obtain a colorless oil (48.3 g). This material was then purified via column chromatography (300 g silica gel, 5-30% MTBE/petroleum ether) to afford ent-2 as a clear liquid [22.6 g, 36% overall mass recovery]: R_{f}=0.59 (1:1 MTBE/petroleum ether); ¹H NMR (CDCl₃, 500 MHz) δ 5.85 (ddd, J=17.0, 10.5, 6.2 Hz, 1H), 5.40 (dt, J=17.3, 1.3 Hz, 1H), 5.27 (dt, J=10.5, 1.3 Hz, 1H), 4.36-4.33 (m, 1H), 3.10 (ddd, J=3.8, 3.8, 3.0 Hz, 1H), 2.81 (dd, J=2.9, 5.0 Hz, 1H), 2.76 (dd, 4.1, 5.0 Hz, 1H), 2.07 (d, J=3.0 Hz, 1H).

### Synthesis of Compound 3

The reaction was carried out at 20-g scale of alcohol following a literature procedure (J. Org. Chem. 2009, 74(15), 5758-5761). A 2-L round-bottomed flask equipped with a mechanical stirrer, a thermocouple probe, and an addition funnel was charged with a solution of epoxy alcohol ent-2 [20 g, 200 mmol, 1 equiv] in tetrahydrofuran (400 mL, 20 vol) along with Ph₃P (105 g, 400 mmol, 2 equiv), and 4-nitrobenzoic acid (67 g, 400 mmol, 2 equiv) under a nitrogen atmosphere. DIAD (81 g, 400 mmol, 2 equiv) was added to the reaction mixture using an addition funnel while maintaining the reaction mixture at 0° C. (ice bath). Once the addition of DIAD was complete, the cold bath was removed and the reaction mixture was allowed to come to ambient temperature (23° C.). The reaction mixture was stirred for 1.5 h (all starting material consumed) and then quenched with aqueous NaHCO₃ solution (100 ml, 5 vol) followed by the addition of MTBE (1000 mL, 50 vol). The resulting solution was transferred into a separatory funnel. Brine (100 mL, 5 vol) was added to obtain phase separation. The organic phase was washed with brine (2×20 vol), dried (MgSO₄), and concentrated under vacuum to obtain an oil (296 g). The oil was passed through a silica plug (1 kg) using 10-20% MTBE/heptanes. The crude solid (46 g) was then dissolved into MTBE (20 vol) and washed with NaHCO₃ (3×5 vol), water (2×2 vol), brine (2×2 vol), dried (MgSO₄), concentrated, and further dried to obtain the benzoate ester as a white solid [29 g, 59%: R_{f}=0.56 (1:1 MTBE/heptanes)]; ¹H NMR (CDCl₃, 500 MHz) δ 8.35 (d, J=10.8 Hz, 2H), 8.25 (d, J=10.8 Hz, 2H), 5.97 (ddd, J=17.2, 10.6, 6.2 Hz, 1H), 5.48 (td, J=17.3, 1.2 Hz, 1H), 5.40 (td, J=10.7, 1.1 Hz, 1H), 5.34 (dd, J=5.0, 1.3 Hz, 1H), 3.31 (ddd, J=6.5, 4.1, 2.6 Hz, 1H), 2.93 (dd, J=4.2, 4.2 Hz, 1H), 2.76 (dd, J=4.8, 2.6 Hz, 1H).

The hydrolysis of the benzoate ester was carried out following the literature procedure (J. Org. Chem. 2009, 74(15), 5758-5761). Thus solution of the ester (22.7 g, 91 mmol, 1 equiv) in methanol (340 mL, 15 vol) was treated with an aqueous solution of K₂CO₃ (13.8 g, 100 mmol, 1.1 equiv, in 34 mL, 1.5 vol water) at 10-15° C. The solution immediately turned into a thick slurry. The slurry was stirred at ambient temperature (23° C.) for 3 h (starting material consumed). The reaction mixture was concentrated on a rotary evaporator (at ambient water bath temperature) to −2 vol (45 mL). The thick solution was then reslurried in DCM (454 mL, 20 vol). The slurry was filtered and the solids were washed with DCM (2×5 vol, 2×114 mL). The combined organic filtrate was dried (MgSO₄), filtered, and concentrated to obtain a solid (31 g). The crude material was then purified by column chromatography (silica gel, 10-30% MTBE/petroleum ether) to obtain the desired alcohol 3 as a clear oil [9.24 g, quantitative yield, R_{f}=0.31 (1:1 MTBE/heptanes)]; ¹H NMR (CDCl₃, 300 MHz) δ 5.94 (ddd, J=16.2, 10.6, 5.5, 1H), 5.40 (d, J=17.3 Hz, 1H), 5.26 (d, J=10.6 Hz, 1H), 4.0 (t, J=5.3 Hz, 1H), 3.07 (m, 1H), 2.84 (t, J=4.8 Hz, 1H), 2.77-2.74 (m, 1H), 2.57 (br s, 1H).

### Synthesis of Compound 4

A 1-L three-necked round-bottomed flask equipped with an addition funnel, an overhead mechanical stirrer, a nitrogen inlet/outlet, was charged with alcohol 3 [9.24 g, 92.3 mmol, 1 equiv] in anhydrous tetrahydrofuran (166 mL, 18 vol). The solution was cooled to −10 to −15° C. The catalyst Bu₄NI (3.41 g, 9.23 mmol, 10 mol %) was charged into the reactor followed by benzyl bromide (19.1 g, 112 mmol, 1.2 equiv). The resulting solution was stirred for 20 min. Sodium hydride (4.1 g, 1.1 equiv, 60% mineral oil dispersion) was then added to the batch in portions such that the batch temperature was maintained at −10 to −15° C. Once the addition of sodium hydride was complete, the reaction mixture was stirred for an additional 30 min and then the cold bath was removed and reaction mixture brought up to ambient temperature and further stirred for 18 h. The reaction was quenched with aqueous NaHCO₃ (37 mL, 4 vol) while maintaining the temperature at −5 to 0° C. (ice bath). The resulting solution was diluted with MTBE (185 mL, 20 vol), the organic layer was washed with water (2×18 mL, 2×3 vol), brine (1×18 mL, 1×3 vol), dried (MgSO₄), filtered, and concentrated under reduced pressure to obtain crude product as an oil. The synthesis was repeated on 1.98 g scale of alcohol 3. The crude from both the reactions were combined and purified via column chromatography (silica gel column, 2.5-10% MTBE/heptanes) to obtain the desired benzylated product 4 as an oil [13.96 g, 65%: R_{f}=0.61 (3:7 MTBE/heptanes)]; ¹H NMR (CDCl₃, 500 MHz) δ 7.36-7.32 (m, 4H), 7.29-7.26 (m, 1H), 5.83 (ddd, J=17.3, 10.5, 6.7, 1H), 5.36 (td, J=17.3, 1.4 Hz, 1H), 5.31 (td, J=10.5, 1.2 Hz, 1H), 4.63 (ABq, J=12.0 Hz, 2H), 3.62 (ddd, J=, 1H), 3.11-3.08 (m, 1H), 2.78 (t, J=4.4 Hz, 1H), 2.60 (dd, J=5.0, 2.7 Hz, 1H).

### Synthesis of Compound 5

A 250-mL round-bottomed flask equipped with a reflux condenser was charged with alcohol 4 [10 g, 52.5 mmol, 1 equiv], phthalimide (11.6 g, 78.8 mmol, 1.5 equiv), pyridine (0.85 mL, 10.5 mmol, 20 mol %) and IPA (100 mL, 10 vol) and the resulting solution was stirred at 80-82° C. for 8 hrs. The reaction mixture was then cooled to ambient temperature and concentrated on a rotatory evaporator to dryness. The residue was adsorbed on silica gel (20 g), dried under high vacuum and then purified by flash column chromatography on silica gel (10-40% MTBE/heptanes) to afford the desired phthalimide protected amino alcohol 5 as a white tacky solid [15.85 g, 89%]: R_{f}=0.34 (1:1 MTBE/heptanes); ¹H NMR (DMSO-d₆, 500 MHz) δ 7.84-7.82 (m, 4H), 7.36-7.31 (m, 4H), 7.28-7.25 (m, 1H), 5.93 (ddd, J=17.5, 10.5, 10.1 Hz, 1H), 5.38-5.35 (m, 2H), 5.12 (d, J=5.5 Hz, 1H), 4.53 (d, J=11.9 Hz, 1H), 4.40 (d, J=11.9 Hz, 1H), 3.98 (dddd, J=9.0, 4.5, 4.5, 4.5 Hz 1H), 3.86 (dd, J=5.8, 4.6 Hz, 1H), 3.67 (dd, J=13.7, 8.9 Hz, 1H), 3.59 (dd, J=13.7, 4.4 Hz, 1H).

### Synthesis of Compound 6

A 1-L three-necked round-bottomed flask equipped with an addition funnel, an overhead mechanical stirrer, and a nitrogen inlet/outlet was charged with a solution of alcohol 5 [15 g, 44.5 mmol, 1 equiv] in anhydrous tetrahydrofuran (270 mL, 18 vol). The solution was cooled to −10 to −15° C., then Bu₄NI (1.64 g, 4.45 mmol, 10 mol %) was charged into the reactor followed by benzyl bromide (9.2 g, 53.8 mmol, 1.2 equiv). The resulting solution was stirred for 20 min, then sodium hydride (1.97 g, 1.1 equiv, 60% mineral oil dispersion) was added to the batch in portions such that the batch temperature was maintained at −10 to −15° C. Once the addition of sodium hydride was complete, the reaction mixture was stirred for an additional 30 min and then brought to ambient temperature and further stirred for 18 h. The reaction was quenched with aqueous NaHCO₃ (60 mL, 4 vol) while maintaining the reaction mixture at −5 to 0° C. (ice bath). The reaction mixture was then diluted with MTBE (300 mL, vol) and the phases separated. The organic layer was washed with water (2×45 mL, 2×3 vol), brine (1×45 mL, 1×3 vol), dried (MgSO₄), filtered, and concentrated to obtain the crude product as an oil. The synthesis was repeated on 1.75 g scale of alcohol 5. The combined crude products from both reactions were purified by flash column chromatography on silica gel (5-25% MTBE/heptanes) to obtain the desired product 6 as a semi solid [15.1 g, 71%: R_{f}=0.61 (1:1 MTBE/heptanes)]; ¹H NMR (CDCl₃, 300 MHz) δ 7.74-7.71 (m, 2H), 7.67-7.64 (m, 2H), 7.37-7.27 (m, 5H), 7.10-7.07 (m, 2H), 6.98-6.93 (m, 3H), 5.97 (ddd, J=17.5, 10.4, 10.0 Hz, 1H), 5.42 (d, J=4.38 Hz, 1H), 5.38 (s, 1H), 4.68 (dd, J=12.3, 12.3 Hz, 2H), 4.45 (d, J=5.37 Hz, 1H), 4.41 (d, J=5.58 Hz, 1H), 3.99-3.82 (m, 3H), 3.65 (dd, J=13.6, 3.2 Hz, 1H).

### Synthesis of Aldehyde 7 and Carboxylic Acid 8

A solution of alkene, 6 [1 g, 2.34 mol] in DCM (60 mL, 60 vol) was sparged with ozone at <−70° C. (dry ice-acetone) for 25 min using house air as oxygen source to generate the ozone. Once the reaction was deemed compete (TLC, 1:1 MTBE/heptanes), the solution was sparged with nitrogen for 20 min to remove residual ozone. The reaction was quenched with dimethyl sulfide (1.7 mL, 23.4 mmol, 10 equiv) while maintaining the reaction mixture at <−70° C. (dry ice-acetone). The cold bath was removed and the mixture was allowed to warm to ambient temperature. The reaction mixture was concentrated under reduced pressure and further dried under high vacuum to obtain the crude aldehyde as a thick oil (1.12 g, >99%, R_{f}=0.36, 1:1 MTBE/heptanes). The reaction was repeated at 13 g scale of 6. The two lots of crude aldehyde were combined and subjected to the Pinnick oxidation without further purification.

The crude aldehyde 7 [14.06 g], was taken into a mixture of tetrahydrofuran, tBuOH, and water (105 mL, 105 mL, 70 mL, 3:3:2, 20 vol) along with NaH₂PO₄ (15.6 g, 130 mmol, 4 equiv) and 2-methyl-2-butene (34.4 mL, 324 mmol, 10 equiv). The solution was cooled (15±5° C., water bath). Sodium chlorite (3.9 g, 43 mmol, 1.33 equiv) was added to the batch and the resulting solution was stirred at ambient temperature for 4 hr. The completion of the reaction was confirmed by TLC analysis (1:1 MTBE/heptanes and 5% MeOH in DCM). The reaction was then quenched with brine (280 mL, 20 vol) and the product extracted into DCM (3×280 mL, 3×20 vol). The organic layers were dried (MgSO₄), concentrated under reduced pressure to obtain the crude acid as a thick oil. The crude acid was purified by flash column chromatography over silica (5-100% MTBE/heptanes followed by 5-20% MeOH/DCM). Fractions containing the acid were combined and concentrated under reduced pressure to afford acid 8 as a white solid [2.64 g, 18%: R_{f}=0.33, 5:95 MeOH/DCM)]; ¹H NMR (CDCl₃, 500 MHz) δ 7.78 (dd, J=5.5, 3.0 Hz, 2H), 7.70 (dd, J=5.5, 3.0 Hz, 2H), 7.43-7.40 (m, 2H), 7.37-7.29 (m, 3H), 7.20-7.19 (m, 2H), 7.14-7.11 (m, 2H), 7.09-7.05 (m, 1H), 4.76 (d, J=11 Hz, 1H), 4.65 (dd, J=10.9, 9.4 Hz, 2H), 4.55 (d, J=11.8 Hz, 1H), 4.13 (ddd, J=6.2, 6.2, 3.1 Hz, 1H), 4.1 (d, J=3.0 Hz, 1H), 3.98 (dd, J=14.2, 6.2 Hz, 1H), 3.89 (dd, J=14.2, 6.2 Hz, 1H).

### Synthesis of Compound 9

A round bottomed flask equipped with a magnetic stirring bar, and a thermocouple probe was charged with a solution of phthalimide-protected amino acid 8 [2.5 g, 5.61 mmol, 1.0 equiv] in THF (28 mL, 11 vol, bulk solvent grade). To the clear, yellow solution was added deionized water (15 mL, 6 vol) and the resulting mixture cooled to 5° C. Methylamine solution in water (5.0 mL, 40 wt %, 56.1 mmol, 10 equiv) was then added to the batch, which was warmed to ambient temperature (21-23° C.) and stirred for 22.5 hours. Analysis of an aliquot from the reaction mixture by LCMS indicated the reaction was complete. The reaction mixture was then concentrated in vacuo to a yellow solid residue, removing all excess methylamine. The residue was taken up in THF (60 mL, 24 vol) and water (30 mL, 12 vol), cooled to 0-5° C., and to the crude amino acid solution was added potassium carbonate (3.9 g, 28.26 mmol, 5.0 equiv), followed by benzylchloroformate (1.4 mL, 9.81 mmol, 1.75 equiv). The batch was warmed to ambient temperature and the reaction allowed to proceed for 25.5 hours. Analysis of an aliquot at this time point by LCMS indicated a complete conversion of the amino acid to the carbamate. The reaction mixture was concentrated under reduced pressure to remove most of THF, the aqueous residue was diluted with water (30 mL, 12 vol) and the pH adjusted with 2N HCl to approximately pH 5 (pH paper strip). The crude product was extracted with chloroform (3×60 mL), the extracts washed with water (1×60 mL) and with aqueous NaCl (1×60 mL), dried (MgSO₄) and concentrated in vacuo to a yellow, mobile oil (3.52 g) which was purified by flash column chromatography on silica gel (50 wt. equiv; elution with 0-5% MeOH in CHCl₃) to afford 9 as a yellow oil, which partially solidified upon further drying under high vacuum [2.22 g, 88.1% yield over two steps]. ¹H NMR (DMSO, 500 MHz) δ 12.92 (s, 1H), 7.43-7.23 (m, 15H), 5.04 (s, 2H), 4.67 (d, J=11.10 Hz, 1H), 4.58 (d, J=11.10 Hz, 1H), 4.48 (d, J=11.05 Hz, 1H), 4.42 (d, J=11.05 Hz, 1H), 4.09 (d, J=2.95 Hz, 1H), 3.96 (ddd, J=6.30, 6.30, 3.15 Hz, 1H), 3.29 (dd, J=6.30, 6.30, 2H).

### Synthesis of Cyclopropyl Amino Acids

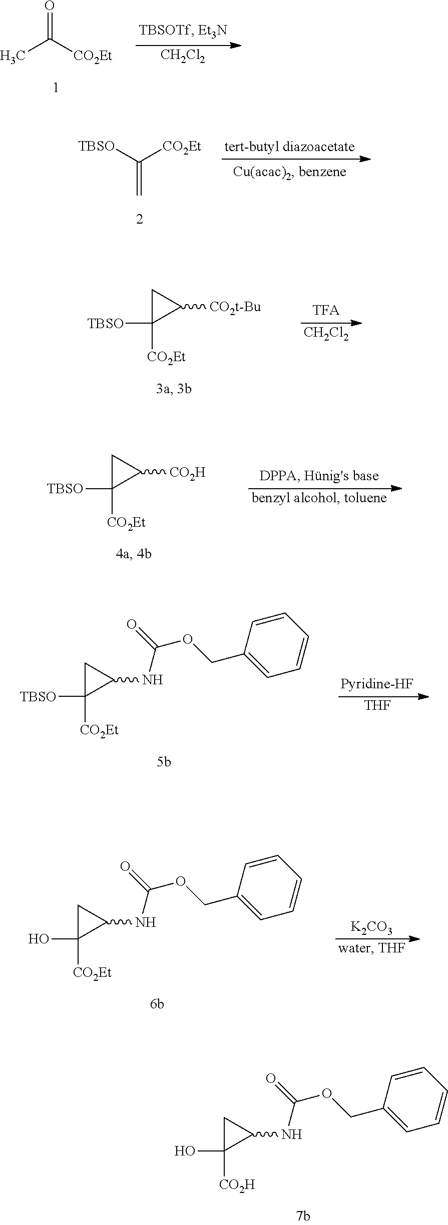

### Ethyl-2-(tert-Butyldimethylsilyloxy)acrylate (2)

A solution of ester 1 (4.00 g, 34.4 mmol) and triethylamine (4.79 mL, 34.4 mmol) in anhydrous dichloromethane (170 mL) was cooled to 0° C. under nitrogen and tert-butyldimethylsilyltrifluoromethane sulfonate (8.31 mL, 36.2 mmol) was added dropwise. The resulting solution was stirred vigorously at reflux for 4 h. The solvent was then carefully evaporated, the residue was dissolved in Et₂O (170 mL), and the organic phase was washed with water (3×50 mL). The organic phase was dried (Na₂SO₄), filtered, and concentrated. The residue was purified by silica gel chromatography eluting with 0-20% diethyl ether/hexanes to afford 2 (4.89 g, 62%) as a clear oil: ¹H NMR (500 MHz, CDCl₃) δ 5.50 (d, J=1.0 Hz, 1H), 4.85 (d, J=1.0 Hz, 1H), 4.21 (q, J=7.0 Hz, 2H), 1.31 (t, J=7.0 Hz, 3H), 0.95 (s, 9H), 0.16 (s, 6H).

### 2-tert-Butyl-1-Ethyl-1-(tert-butyldimethylsilyloxy)cyclopropane-1,2-dicarboxylate (3a and 3b)

A mixture of ethyl-2-(tert-butyldimethylsilyloxy)acrylate (2, 500 mg, 2.17 mmol) and Cu(acac)₂ (0.011 g, 0.043 mmol) was heated at 80° C. A solution of tert-butyl diazoacetate (463 mg, 3.25 mmol) in benzene (5 mL) was added to the reaction mixture over 2 h. After this time, the reaction mixture was cooled to room temperature and concentrated. The residue was purified by silica gel chromatography eluting with 0-10% diethyl ether/hexanes to afford both diastereomers 3a (0.119 g, 16%) and 3b (0.235 g, 31%) as clear oils. 3a: ¹H NMR (500 MHz, CDCl₃) δ 4.25-4.13 (m, 2H), 2.28 (dd, J=7.5, 2.0 Hz, 1H), 1.73 (dd, J=7.5, 2.0 Hz, 1H), 1.59 (dd, J=9.5, 4.0 Hz, 1H), 1.46 (s, 9H), 1.29 (t, J=7.5 Hz, 3H), 0.90 (s, 9H), 0.18 (s, 3H), 0.12 (s, 3H); ESI MS m/z 367 [M+Na]⁺; 3b: ¹H NMR (500 MHz, CDCl₃) δ 4.23 (dq, J=11.0, 7.0 Hz, 1H), 4.13 (dq, J=11.0, 7.0 Hz, 1H), 2.11 (dd, J=10.0, 1.5 Hz, 1H), 1.85 (dd, J=5.5, 2.5 Hz, 1H), 1.43 (s, 9H), 1.54 (dd, J=10.0, 4.0 Hz, 1H), 1.28 (t, J=7.5 Hz, 3H), 0.86 (s, 9H), 0.19 (s, 3H), 0.18 (s, 3H); ESI MS m/z 367 [M+Na]⁺.

### 2-(tert-Butyldimethylsilyloxy)-2-(ethoxycarbonyl)cyclopropanecarboxylic Acid (4a and 4b)

A mixture of dicarboxylate 3a and 3b (0.385 g, 1.12 mmol, 1:2 ratio of 3a/3b), trifluoroacetic acid (0.43 mL), and dichloromethane (0.5 mL) was stirred overnight at room temperature. The solids were filtered, and the filtrate was concentrated. The residue was purified by silica gel chromatography eluting with 0-100% diethyl ether/hexanes to afford both diastereomers 4a (0.050 g, 15%) and 4b (0.078 g, 24%) as off-white solids. 4a: ¹H NMR (500 MHz, CDCl₃) δ 4.25-4.17 (m, 2H), 2.38 (dd, J=7.5, 1.5 Hz, 1H), 1.81-1.76 (m, 2H), 1.30 (t, J=7.0 Hz, 3H), 0.90 (s, 9H), 0.21 (s, 3H), 0.13 (s, 3H); ESI MS m/z 289 [M+H]⁺; 4b: ¹H NMR (500 MHz, CDCl₃) δ 4.22 (q, J=7.0 Hz, 1H), 2.21 (dd, J=10.0, 1.5 Hz, 1H), 1.93 (dd, J=8.0, 2.0 Hz, 1H), 1.52 (dd, J=6.0, 3.5 Hz, 1H), 1.28 (t, J=7.0 Hz, 3H), 0.87 (s, 9H), 0.19 (s, 3H), 0.17 (s, 3H); ESI MS m/z 287 [M−H]⁻.

### Ethyl-2-(Benzyloxycarbonylamino)-1-(tert-butyldimethylsilyloxy)cyclopropanecarboxylate (5b)

A mixture of 2-(tert-butyldimethylsilyloxy)-2-(ethoxycarbonyl)cyclopropanecarboxylic acid (4b, 0.335 g, 1.16 mmol) in toluene (5 mL) under nitrogen was treated with Hünig's base (0.260 mL, 1.51 mmol) and the mixture was cooled to 0° C. After this time, DPPA (0.324 mL, 1.51 mmol) was added and the mixture was heated at 90° C. for 30 min, followed by the addition of benzyl alcohol (0.155 mL, 1.51 mmol). After 15 h, the mixture was cooled, diluted with ethyl acetate (75 mL), and washed sequentially with 10% citric acid (2×50 mL), water (50 mL), and saturated NaHCO₃ (50 mL). The organic phase was dried (MgSO₄), filtered, and concentrated. The residue was purified by silica gel chromatography eluting with 10% EtOAc/hexanes to 100% EtOAc to afford the title compound as a clear oil (0.146 g, 30%): ¹H NMR (300 MHz, CDCl₃) δ 7.34-7.30 (m, 5H), 5.40-5.38 (m, 1H), 5.21-5.00 (m, 2H), 4.29-4.18 (m, 2H), 4.16-4.09 (m, 1H), 1.50-1.47 (m, 2H), 1.30 (t, J=7.2 Hz, 3H), 0.88 (s, 9H), 0.26-0.07 (m, 6H); Multimode (APCI+ESI) MS m/z 295 [M+H]⁺.

### Ethyl 2-(Benzyloxycarbonylamino)-1-hydroxycyclopropanecarboxylate (6b)

To a solution of ethyl 2-(benzyloxycarbonylamino)-1-(tert-butyldimethylsilyloxy)cyclopropanecarboxylate (1.45 g, 3.69 mmol) in THF (35 mL) under N₂ was added HF•pyridine (1.0 mL, 38 mmol). The reaction mixture was stirred for 5 h. After this time, additional HF•pyridine (1.0 mL, 38 mmol) was added and stirring was continued for 19 h. The reaction mixture was then cooled to 0° C. and diluted with Et₂O (150 mL). The mixture was then carefully quenched with saturated aqueous NaHCO₃ until gas evolution ceased. At this time, the organic layer was separated and the remaining aqueous layer was extracted with Et₂O (300 mL). The combined organic layers were washed with brine (200 mL), dried (Na₂SO₄), filtered, and concentrated in vacuo. Purification by silica gel chromatography eluting with 20%-50% EtOAc/hexanes afforded the title compound (0.960 g, 93%): ¹H NMR (300 MHz, CDCl₃) δ 7.34-7.30 (m, 5H), 5.11-4.83 (m, 3H), 4.21 (q, J=7.2 Hz, 2H), 3.37-3.25 (m, 2H), 1.73-1.68 (m, 1H), 1.27 (t, J=7.2 Hz, 3H), 1.14-1.06 (m, 1H); ESI MS m/z 280 [M+H]⁺.

### 2-(Benzyloxycarbonylamino)-1-hydroxycyclopropanecarboxylic acid (7b)

To a 0° C. solution of ethyl 2-(benzyloxycarbonylamino)-1-hydroxycyclopropanecarboxylate (6b, 12.5 g, 44.7 mmol) in THF (100 mL) was added K₂CO₃(24.7 g, 179.0 mmol) as a solution in H₂O (300 mL). The reaction was allowed to warm to room temperature and stirred for 4 h and then additional H₂O (200 mL) was added. After stirring an additional 18 h at room temperature the reaction was concentrated to remove most of the THF. The remaining aqueous solution was washed with Et₂O (2×500 mL), acidified with 2N HCl to pH 2, and then extracted with EtOAc (5×200 mL). The combined EtOAc layers were washed with brine (500 mL), dried (Na₂SO₄), filtered and concentrated in vacuo to afford the title compounds (7.75 g, 69%) as a mixture of diastereomers. The mixture was triturated with Et₂O to afford a white solid as mostly the major diastereomers. The supernatant was concentrated and then triturated with Et₂O to afford a clean mixture of both diastereomers. Major Diastereomer: ¹H NMR (300 MHz, MeOD) δ 7.50-7.14 (m, 5H), 5.22-4.96 (m, 2H), 3.23-3.10 (m, 1H), 1.60 (dd, J=8.9, 6.3 Hz, 1H), 1.10 (t, J=6.2 Hz, 1H); Multimode (APCI+ESI) MS m/z 250 [M−H]⁻. Mixture of Diastereomers: ¹H NMR (300 MHz, MeOD) δ 7.45-7.14 (m, 5H), 5.24-5.01 (m, 2H), 3.25-3.15 (m, 0.46H), 3.14-3.01 (m, 0.54H), 1.71-1.53 (m, 1H), 1.42 (dd, J=9.1, 6.4 Hz, 0.54H), 1.12 (t, J=6.2 Hz, 0.46H); Multimode (APCI+ESI) MS m/z 250 [M−H]⁻.

### REPRESENTATIVE COMPOUNDS

The following representative compounds were, or may be, prepared according to the foregoing procedures.

### SISOMICIN ANALOGS

### Example 1

### 6′-(2-Hydroxy-ethyl)-1-(4-amino-2(S)-hydroxy-butyryl)-sisomicin (Compound 1)

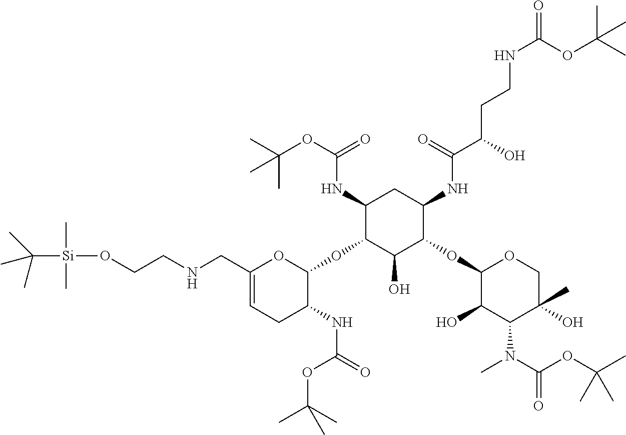

6′-(2-tert-Butyldimethylsililoxy-ethyl)-2′,3,3″-triBoc-1-(N-Boc-4-amino-2(S)-hydroxy-butyryl)-sisomicin

2′,3,3″-triBoc-1-(N-Boc-4-amino-2(S)-hydroxy-butyryl)-sisomicin (0.10 g, 0.105 mmol) was treated with tert-butyldimethylsilyloxy acetaldehyde following Procedure 1-Method A to yield the desired 6′-(2-tert-butyldimethylsilyloxy-ethyl)-2′,3,3″-triBoc-1-(N-Boc-4-amino-2(S)-hydroxy-butyryl)-sisomicin (MS m/e [M+H]⁺ Calcd 1107.6. Found 1107.4), which was carried through to the next step without further purification.

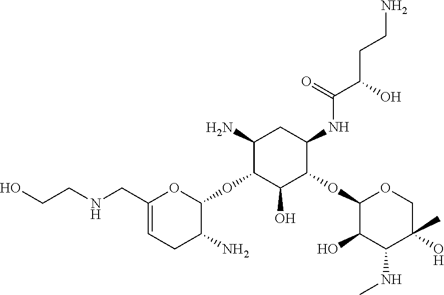

### 6′-(2-Hydroxy-ethyl)-1-(4-amino-2(S)-hydroxy-butyryl)-sisomicin

6′-(2-tert-butyldimethylsililoxy-ethyl)-2′,3,3″-triBoc-1-(N-Boc-4-amino-2(S)-hydroxy-butyryl)-sisomicin (0.105 mmol) was submitted to Procedure 3-Method B for Boc removal to yield a crude, which was purified by RP HPLC Method 1-Column A to yield 6′-(2-hydroxy-ethyl)-1-(4-amino-2(S)-hydroxy-butyryl)-sisomicin: MS m/e [M+H]⁺ Calcd 593.3. Found 593.2, [M+Na]⁺ 615.3; CLND 97.5% purity.
Example 2. 6′-(2-Hydroxy-ethyl)-1-(4-amino-2(R)-hydroxy-butyryl)-sisomicin
Example 3. 6′-(2-Hydroxy-propanol)-1-(4-amino-2(R)-hydroxy-butyryl)-sisomicin
Example 4. 6′-(Methyl-piperidin-4-yl)-1-(4-amino-2(R)-hydroxy-butyryl)-sisomicin
Example 5. 6′-(Methyl-cyclopropyl)-1-(4-amino-2(R)-hydroxy-butyryl)-sisomicin
Example 6. 6′-(3-Amino-propyl)-1-(4-amino-2(R)-hydroxy-butyryl)-sisomicin
Example 7. 6′-Methyl-cyclopropyl-1-(3-amino-2(R)-hydroxy-propionyl)-sisomicin
Example 8. 6′-Methyl-piperidinyl-1-(3-amino-2(R)-hydroxy-propionyl)-sisomicin
Example 9. 6′-(2-Hydroxy-ethyl)-1-(3-amino-2(R)-hydroxy-propionyl)-sisomicin
Example 10. 6′-(2-Hydroxy-propanol)-1-(3-amino-2(R)-hydroxy-propionyl)-sisomicin
Example 11. 6′-(3-Amino-propyl)-1-(3-amino-2(R)-hydroxy-propionyl)-sisomicin
Example 12. 6′-(Methyl-piperidin-4-yl)-1-(4-amino-2(S)-hydroxy-butyryl)-sisomicin
Example 13. 6′-(Methyl-cyclopropyl)-1-(3-amino-2(S)-hydroxy-propionyl)-sisomicin
Example 14. 6′-(2-Hydroxy-propanol)-1-(3-amino-2(S)-hydroxy-propionyl)-sisomicin
Example 15. 6′-(Methyl-piperidin-4-yl)-1-(3-amino-2(S)-hydroxy-propionyl)-sisomicin
Example 16. 6′-(2-Hydroxy-ethyl)-1-(3-amino-2(S)-hydroxy-propionyl)-sisomicin
Example 17. 6′-(3-Amino-propyl)-1-(3-amino-2(S)-hydroxy-propionyl)-sisomicin
Example 18. 6′-(Methyl-cyclopropyl)-1-(4-amino-2(S)-hydroxy-butyryl)-sisomicin
Example 19. 6′-(2-Hydroxy-propanol)-2′,3-diPNZ-1-(N-Boc-4-amino-2(S)-hydroxy-butyryl)-sisomicin
Example 20. 6′-(3-Amino-2-hydroxy-propionyl)-1-(3-amino-2(S)-hydroxy-propionyl)-sisomicin
Example 21. 6′-(2-Hydroxy-3-propionamide)-1-(3-amino-2(S)-hydroxy-propionyl)-sisomicin
Example 22. 6′-(3-Amino-2-hydroxy-propyl)-1-(3-amino-2(S)-hydroxy-propionyl)-sisomicin
Example 23. 6′-(2-Hydroxy-propanol)-1-(2-hydroxy-acetyl)-sisomicin
Example 24. 6′-(3-Amino-propyl)-1-(2-hydroxy-acetyl)-sisomicin
Example 25. 6′-(2-Hydroxy-ethyl)-1-(2-hydroxy-acetyl)-sisomicin
Example 26. 6′-(3-Amino-propyl)-1-(2-amino-ethylsulfonamide)-sisomicin
Example 27. 6′-(2-Hydroxy-propanol)-1-(2-amino-ethylsulfonamide)-sisomicin
Example 28. 6′-(2(S)-Hydroxy-propanol)-1-(4-amino-2(S)-hydroxy-butyryl)-sisomicin
Example 29. 6′-(2-Hydroxy-ethyl)-1-(2-amino-ethylsulfonamide)-sisomicin
Example 30. 6′-(2-Amino-propanol)-1-(4-amino-2(S)-hydroxy-butyryl)-sisomicin
Example 31. 6′-(4-Hydroxy-piperidin-4-yl)-methyl)-1-(4-amino-2(S)-hydroxy-butyryl)-sisomicin
Example 32. 6′-(2-Hydroxy-5-amino-pentyl)-1-(4-amino-2(S)-hydroxy-butyryl)-sisomicin
Example 33. 6′-(Methyl-trans-3-amino-cyclobutyl)-1-(4-amino-2(S)-hydroxy-butyryl)-sisomicin
Example 34. 6′-(2-Hydroxy-ethyl)-1-(3-hydroxy-pyrrolidin-3-yl-acetyl)-sisomicin
Example 35. 6′-(2-Hydroxy-4-amino-butyl)-1-(3-hydroxy-pyrrolidin-3-yl-acetyl)-sisomicin
Example 36. 6′-(Methyl-cyclopropyl)-1-(3-hydroxy-azetidin-3-yl-acetyl)-sisomicin
Example 37. 6′-(2-Hydroxy-ethyl)-1-(3-hydroxy-azeti din-3-yl-acetyl)-sisomicin
Example 38. 6′-(2-Amino-ethyl)-1-(4-amino-2(S)-hydroxy-butyryl)-sisomicin
Example 39. 6′-(Methyl-(1-hydroxy-3-methylamino-cyclobutyl)-1-(4-amino-2(S)-hydroxy-butyryl)-sisomicin
Example 40. 6′-(3-Amino-propyl)-1-(3-hydroxy-pyrrolidin-3-yl-acetyl)-sisomicin
Example 41. 6′-(Methyl-cyclopropyl)-1-(3-hydroxy-pyrrolidin-3-yl-acetyl)-sisomicin
Example 42. 6′-(2-Hydroxy-3-amino-propyl)-1-(3-hydroxy-pyrrolidin-3-yl-acetyl)-sisomicin
Example 43. 6′-(4-Amino-butyl)-1-(4-amino-2(S)-hydroxy-butyryl)-sisomicin
Example 44. 6′-(5-Amino-pentyl)-1-(4-amino-2(S)-hydroxy-butyryl)-sisomicin
Example 45. 6′-(Ethyl-2-(1-methylpiperazin-2-yl)-1-(4-amino-2(S)-hydroxy-butyryl)-sisomicin
Example 46. 6′-(Methyl-(1-hydroxy-3-amino-cyclobutyl)-1-(4-amino-2(S)-hydroxy-butyryl)-sisomicin
Example 47. 6′-(Methyl-(1-hydroxy-3-amino-cyclobutyl)-1-(3-hydroxy-azetidin-3-yl-acetyl)-sisomicin
Example 48. 6′-(3-Amino-propyl)-1-(4-amino-2(S)-hydroxy-butyryl)-sisomicin
Example 49. 6′-(Methyl-pyrrolidin-2-yl)-1-(4-amino-2(S)-hydroxy-butyryl)-sisomicin
Example 50. 6′-(2(S)-Hydroxy-3-propanoic)-1-(4-amino-2(S)-hydroxy-butyryl)-sisomicin
Example 51. 6′-(2,2-Dimethyl-3-amino-propyl)-1-(3-amino-2(S)-hydroxy-propionyl)-sisomicin
Example 52. 6′-(3-Amino-3-cyclopropyl-propyl)-1-(3-amino-2(S)-hydroxy-propionyl)-sisomicin
Example 53. 6′-(Methyl-4(S)-hydroxy-pyrrolidin-2(R)-yl)-1-(3-amino-2(S)-hydroxy-propionyl)-sisomicin
Example 54. 6′-(3-Propanol)-1-(3-amino-2(S)-hydroxy-propionyl)-sisomicin
Example 55. 6′-(2-Methyl-2-amino-propyl)-1-(3-amino-2(S)-hydroxy-propionyl)-sisomicin
Example 56. 6′-(Methyl-1-amino-cyclobutyl)-1-(3-amino-2(S)-hydroxy-propionyl)-sisomicin
Example 57. 6′-(3-Amino-propyl)-1-(3-hydroxy-azetidin-3-yl-acetyl)-sisomicin
Example 58. 6′-(3-Amino-propyl)-1-(1-hydroxy-3-amino-cyclobutyl-acetyl)-sisomicin
Example 59. 6′-(Methyl-trans-3-amino-cyclobutyl)-1-(3-amino-2(S)-hydroxy-propionyl)-sisomicin
Example 60. 6′-(Methyl-trans-3-amino-cyclobutyl)-1-(1-hydroxy-3-amino-cyclobutyl-acetyl)-sisomicin
Example 61. 6′-Methyl-1-(3-hydroxy-azetidin-3-yl-acetyl)-sisomicin
Example 62. 6′-(2-Hydroxy-ethyl)-1-(1-hydroxy-3-amino-cyclobutyl-acetyl)-sisomicin
Example 63. 6′-(Methyl-trans-3-amino-cyclobutyl)-1-(3-hydroxy-azetidin-3-yl-acetyl)-sisomicin
Example 64. 6′-Methyl-1-(1-hydroxy-3-amino-cyclobutyl-acetyl)-sisomicin
Example 65. 6′-(Methyl-4(S)-amino-pyrrolidin-2(S)-yl)-1-(3-amino-2(S)-hydroxy-propionyl)-sisomicin
Example 66. 6′-(Methyl-1-aminomethyl-cyclopropyl)-1-(3-amino-2(S)-hydroxy-propionyl)-sisomicin
Example 67. 6′-(Methyl-1-Amino-cyclopropyl)-1-(3-amino-2(S)-hydroxy-propionyl)-sisomicin
Example 68. 6′-(2-Hydroxy-4-amino-butyl)-1-(3-amino-2(S)-hydroxy-propionyl)-sisomicin
Example 69. 6′-(Methyl-1(R)-amino-2(S)-hydroxy-cyclopent-4(S)-yl)-1-(3-amino-2(S)-hydroxy-propionyl)-sisomicin
Example 70. 6′-(Ethyl-2-(3-hydroxy-azetidin-3-yl))-1-(3-amino-2(S)-hydroxy-propionyl)-sisomicin
Example 71. 6′-Methylcyclopropyl-1-(2-(azetidin-3-yl)-2-hydroxy-acetyl)-sisomicin
Example 72. 6′-(Methyl-trans-3-amino-cyclobutyl)-1-(2-(azetidin-3-yl)-2-hydroxy-acetyl)-sisomicin
Example 73. 6′-(Methyl-azetidin-3-yl)-1-(3-amino-2(S)-hydroxy-propionyl)-sisomicin
Example 74. 6′-(Methyl-1-aminomethyl-cyclopropyl)-1-(2-(azetidin-3-yl)-2-hydroxy-acetyl)-sisomicin
Example 75. 6′-(2-Hydroxy-ethyl)-1-(2-(azetidin-3-yl)-2-hydroxy-acetyl)-sisomicin
Example 76. 6′-(3-Amino-propyl)-1-(2-(azetidin-3-yl)-2-hydroxy-acetyl)-sisomicin
Example 77. 6′-(2-Hydroxy-4-amino-butyl)-1-(2-(azetidin-3-yl)-2-hydroxy-acetyl)-sisomicin
Example 78. 6′-(Methyl-trans-3-amino-cyclobutyl)-1-(3-hydroxy-pyrrolidin-3-yl-acetyl)-sisomicin
Example 79. 6′-(Methyl-1-aminomethyl-cyclopropyl)-1-(3-hydroxy-pyrrolidin-3-yl-acetyl)-sisomicin
Example 80. 6′-(4-Hydroxy-5-amino-pentyl)-1-(3-amino-2(S)-hydroxy-propionyl)-sisomicin
Example 81. 6′-(N-(Azetidin-3-yl)-2-amino-ethyl)-1-(3-amino-2(S)-hydroxy-propionyl)-sisomicin
Example 82. 6′-(2-Hydroxy-3-amino-propyl)-1-(2-(azetidin-3-yl)-2-hydroxy-acetyl)-sisomicin
Example 83. 6′-(Methyl-3-amino-1-hydroxy-cyclobutyl)-1-(2-(azetidin-3-yl)-2-hydroxy-acetyl)-sisomicin
Example 84. 2′-(Methyl-pyrrolidin-3-yl)-1-(4-amino-2(S)-hydroxy-butyryl)-sisomicin
Example 85. 2′-(Methyl-pyrrolidin-2-yl)-1-(4-amino-2(S)-hydroxy-butyryl)-sisomicin
Example 86. 2′-(N-Methyl-amino-acetyl)-1-(4-amino-2(S)-hydroxy-butyryl)-sisomicin
Example 87. 2′-(2-Amino-acetyl)-1-(4-amino-2(S)-hydroxy-butyryl)-sisomicin
Example 88. 2′-(2-Amino-propionyl)-1-(4-amino-2(S)-hydroxy-butyryl)-sisomicin
Example 89. 2′-(3-Amino-2-hydroxy-propionyl)-1-(4-amino-2(S)-hydroxy-butyryl)-sisomicin
Example 90. 2′-(Pyrrolidin-2-yl-acetyl)-1-(4-amino-2(S)-hydroxy-butyryl)-sisomicin
Example 91. 2′-(3-Amino-propyl)-1-(4-amino-2(S)-hydroxy-butyryl)-sisomicin
Example 92. 2′-(Morpholin-2-yl-acetyl)-1-(4-amino-2(S)-hydroxy-butyryl)-sisomicin
Example 93. 2′-(2-Amino-ethyl-sulfonamide)-1-(4-amino-2(S)-hydroxy-butyryl)-sisomicin
Example 94. 2′-(N,N-Dimethyl-2,2-dimethyl-3-amino-propyl)-1-(4-amino-2(S)-hydroxy-butyryl)-sisomicin
Example 95. 2′-(2(S)-Amino-propyl)-1-(4-amino-2(S)-hydroxy-butyryl)-sisomicin
Example 96. 2′-(Azetidin-3-yl)-1-(4-amino-2(S)-hydroxy-butyryl)-sisomicin
Example 97. 2′-(2-Amino-propanol)-1-(4-amino-2(S)-hydroxy-butyryl)-sisomicin
Example 98. 2′-(2-Hydroxy-ethyl)-1-(4-amino-2(S)-hydroxy-butyryl)-sisomicin
Example 99. 2′-(2,5-Diamino-pentoyl)-1-(4-amino-2(S)-hydroxy-butyryl)-sisomicin
Example 100. 2′-(2-Hydroxy-propanol)-1-(4-amino-2(S)-hydroxy-butyryl)-sisomicin
Example 101. 2′-(2-Hydroxy-3-amino-propyl)-1-(4-amino-2(S)-hydroxy-butyryl)-sisomicin
Example 102. 2′-(4-Amino-butyl)-1-(4-amino-2(S)-hydroxy-butyryl)-sisomicin
Example 103. 2′-Guanidinium-1-(4-amino-2(S)-hydroxy-butyryl)-sisomicin
Example 104. 2′-(Methyl-trans-3-amino-cyclobutyl)-1-(4-amino-2(S)-hydroxy-butyryl)-sisomicin
Example 105. 6′,2′-bis-Guanidinium-sisomicin
Example 106. 6′-(2-Hydroxy-ethyl)-2′-guanidinium-sisomicin
Example 107. 6′-(Methyl-trans-3-amino-cyclobutyl)-2′-guanidinium-sisomicin
Example 108. 6′-Methyl-2′-guanidinium-sisomicin

Further Sisomicin Analogs

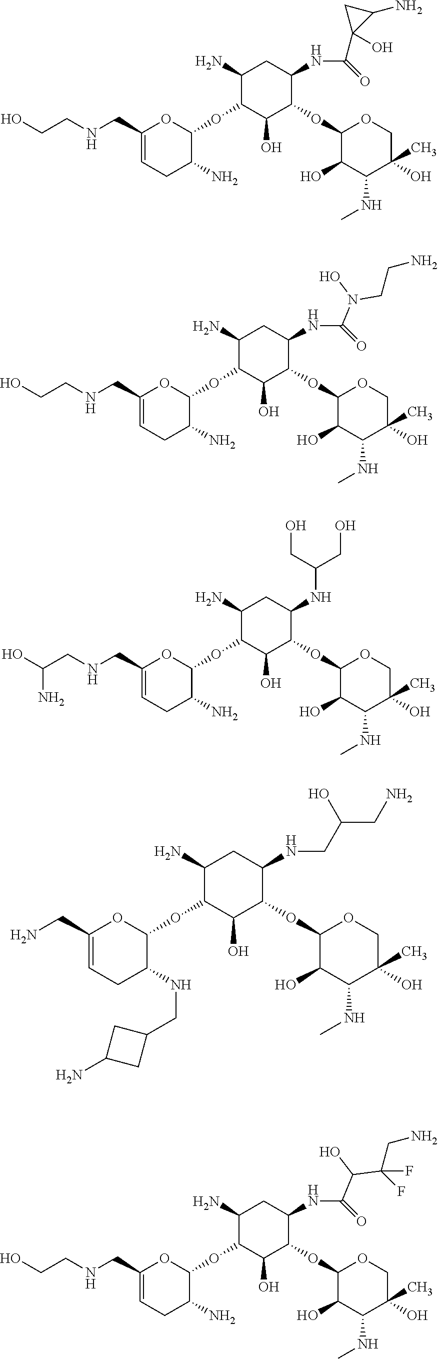

Kanamycin B Analogs

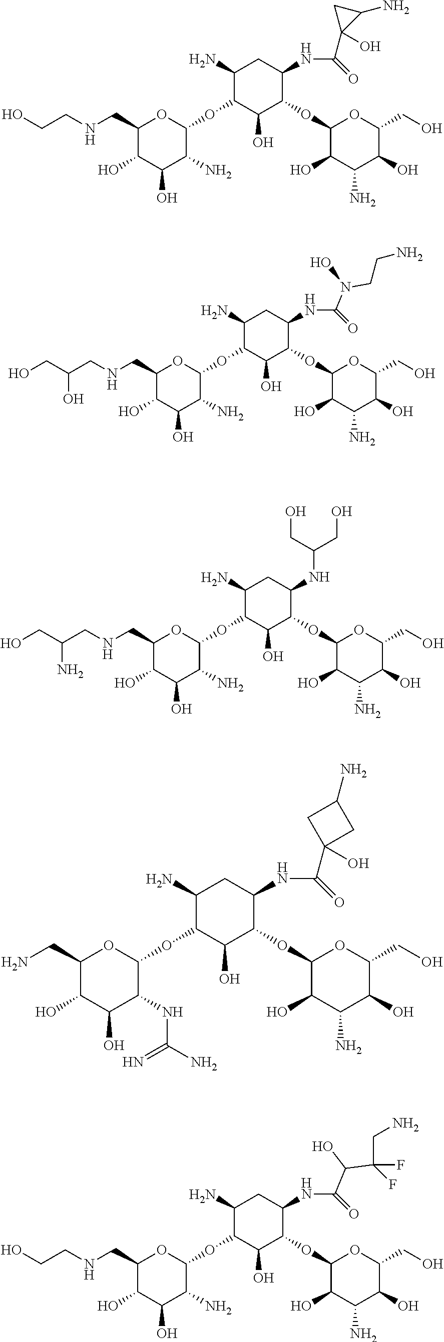

Kanamycin A Analogs

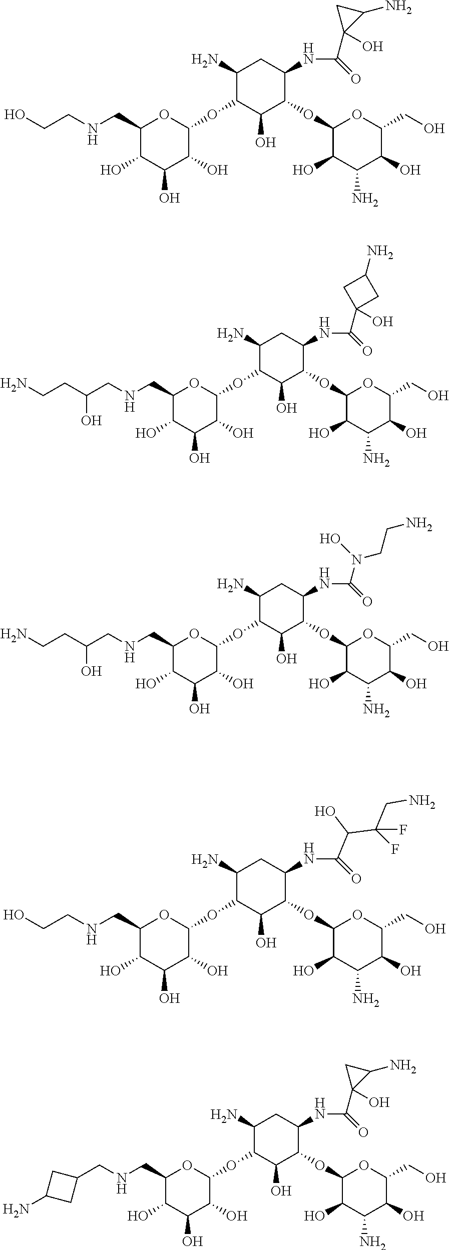

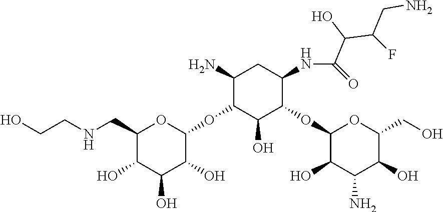

Dibekacin Analogs

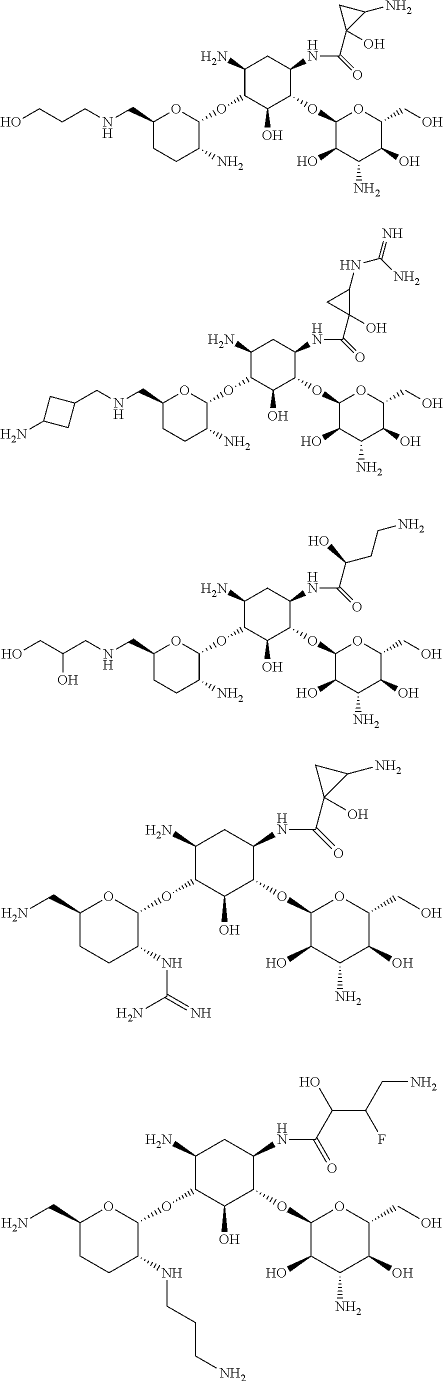

Tobramycin Analogs

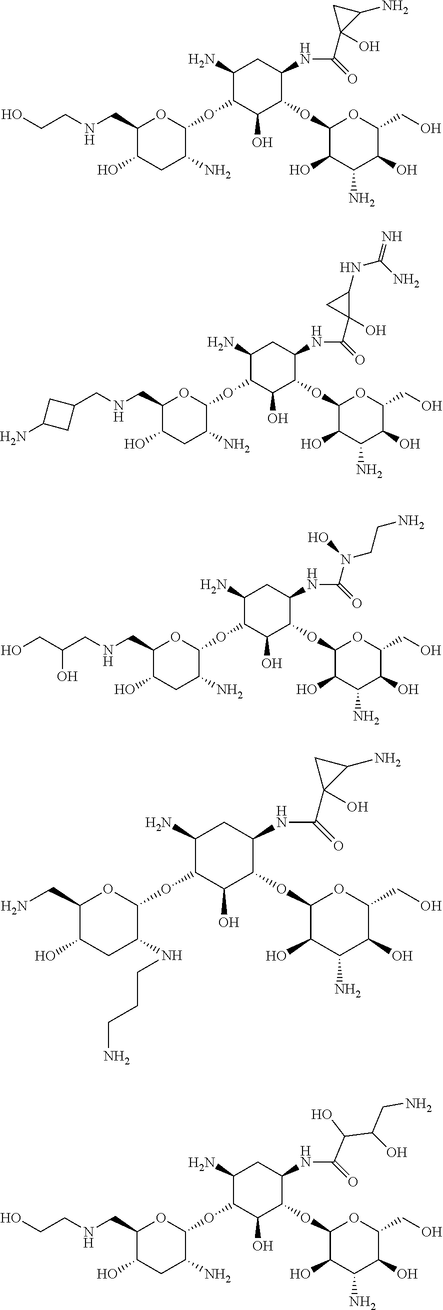

Gentamicin C Analogs

(wherein each R₁₁ is, independently, hydrogen or methyl)

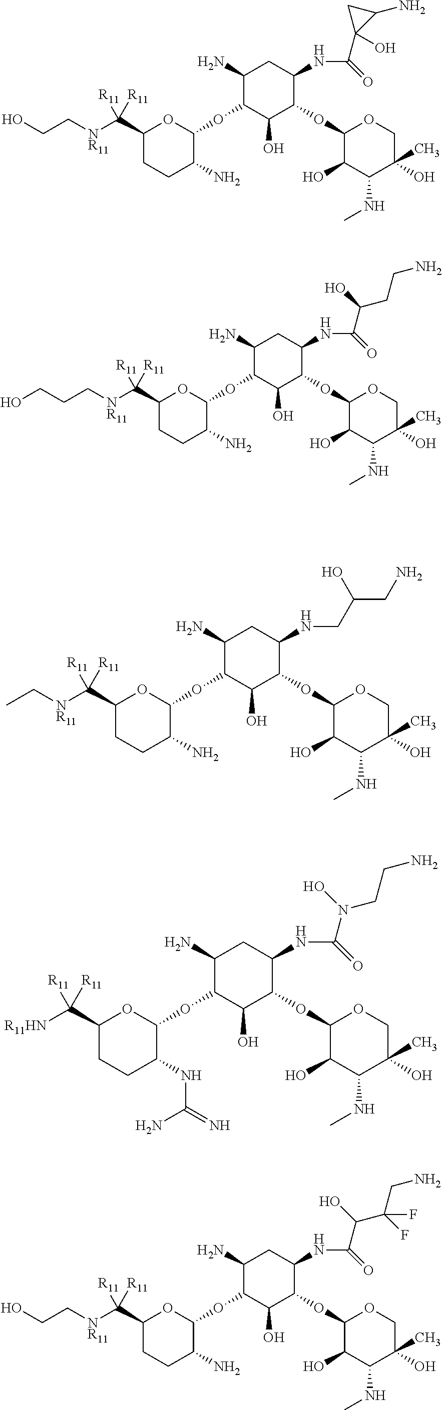

Gentamicin B Analogs

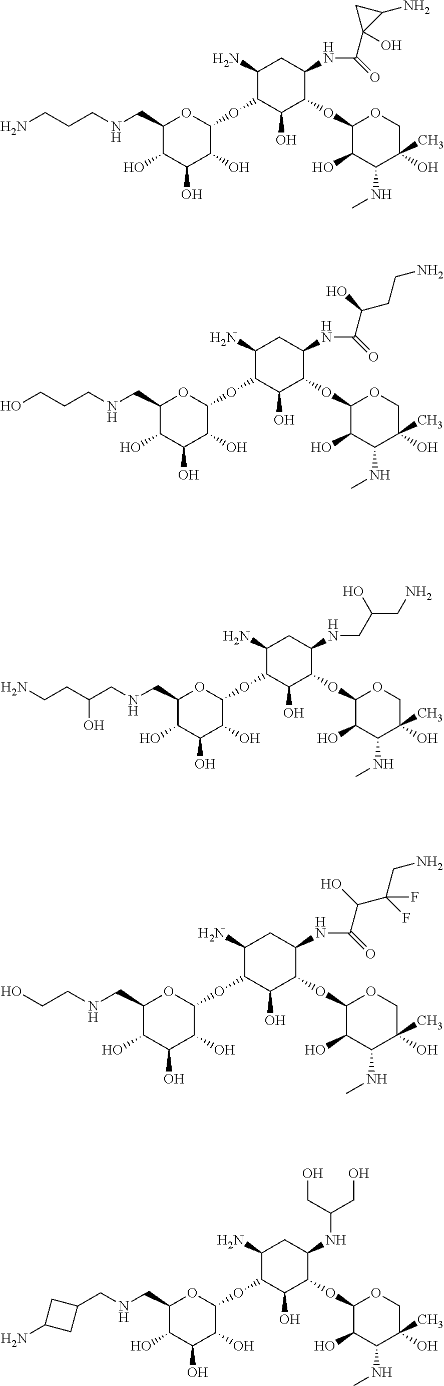

### Biological Example 1

### Pharmacokinetics of Compound 1 in Animals

In vitro and in vivo models have demonstrated that AG bactericidal activity is concentration-dependent; higher concentrations of free drug cause faster killing of exposed bacteria. The ratios of peak serum concentration (Cₘₐₓ) to the minimum inhibitory concentration (MIC) of the target pathogen (Cₘₐₓ/MIC) and area under the time-concentration curve (AUC) to the MIC (AUC/MIC) are important indicators of the bactericidal activity of AGs. As such, the pharmacokinetic (PK) profile, and particularly these ratios are important pharmacodynamic (PD) parameters that predict AG efficacy. The studies described below were performed to determine the PD parameters of the compound shown above in Representative Compounds Example 1 (referred to herein as “Compound 1” or “Cmpd”), in three different types of animals.

Male CD-1 mice, Sprague Dawley rats, and beagle dogs were administered Compound 1 intravenously. Compound 1 was preformulated as a sterile isotonic solution of 10 mg/mL in PBS. Specifically, male CD-1 mice were administered a single bolus IV injection of the Compound at 10 mg/kg, via a bolus IV injection into the tail vein. Two blood samples per mouse were collected by retro-orbital puncture into tubes containing the anticoagulant K₂EDTA prior to dosing and at 0.033, 0.083, 0.25, 0.5, 1 and 24 hours. Rats were administered Compound 1 at 10 mg/kg via a bolus IV injection into the tail vein. Twelve blood samples per rat were collected via a jugular vein cannula into tubes containing the anticoagulant K₂EDTA at various timepoints (i.e., 0.033, 0.083, 0.25, 0.5, 1, 2, 4, 6, 8, 10, 12, and 24 hours). Male beagle dogs were administered Compound 1 at 10 mg/kg through a temporary percutaneous catheter placed in a peripheral vein. Whole blood samples (0.5 mL) were collected from each dog prior to dosing and at 0.033, 0.083, 0.25, 0.5, 1, 2, 4, 6, 8, and 24 hours via direct venipuncture of a jugular or cephalic vein into tubes containing the anticoagulant K₂EDTA. A sparse sampling method was employed to conserve animals and provide the best data possible from fewer mice. Samples were centrifuged, and plasma was collected and transferred into a new tube and frozen at −80° C. until they were analyzed.

Blood, urine, and kidney samples collected over 24 hours were analyzed for Compound 1 levels using LC-MS/MS. Method performance was acceptable in the concentration ranges necessary to determine plasma levels. Compound 1 plasma concentrations were determined by comparing Compound 1 concentration in the sample to a standard curve derived from LC-MS/MS measurements of the analyte. The standard curve fit was linear with a 1/x² weighting.

Bioanalytical data analyses were carried out using Analyst software (version 1.4.1, Applied Biosystems, Toronto, ON, Canada). WinNonLin software (version 5.2, Pharsight, Mountain View, Calif.) was used to calculate PK parameters using NCA model 201 (IV bolus) with sparse sampling. PK parameters were determined by noncompartmental analysis using WinNonLin. Plasma protein binding was determined using equilibrium dialysis. In vitro stability was determined using commercially available plasma, microsomes and hepatocytes.

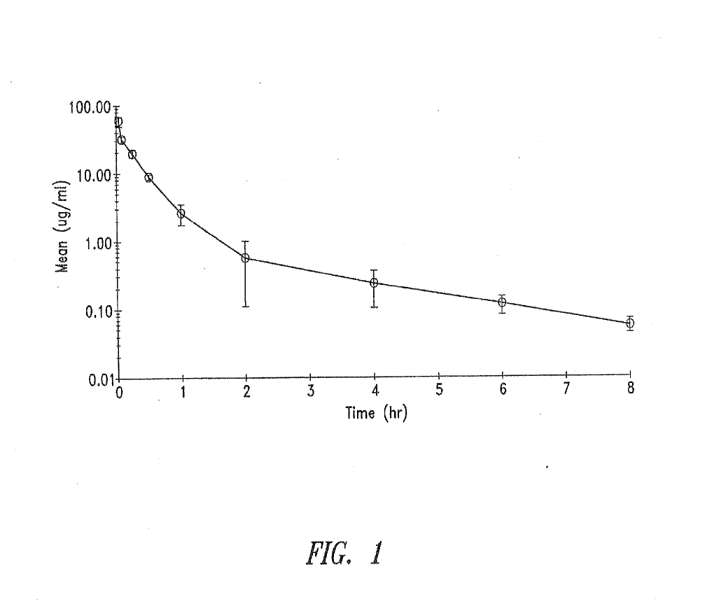

The average plasma concentrations observed in mice at each timepoint up to eight hours following administration are shown in FIG. 1. The PK parameters estimated for Compound 1 administered at 10 mg/kg as a bolus IV injection in mice are shown in Table 1.

TABLE 1
PK Parameter Estimates for a Single Bolus IV Injection
of Compound 1 Administered at 10 mg/kg in Mice

| | No./ | C₀ | AUC_{0-∞} | t_{1/2} | CL | MRT_{0-∞} | Vₛₛ |
|---|---|---|---|---|---|---|---|
| | Sex | (μg/mL) | (hr*μg/mL) | (hr) | (mL/hr/kg) | (hr) | (mL) |
| Mouse ^{a} | 15 M | 87.6 | 18.5 | 1.4 | 541 | 0.6 | 340 |

AUC_{0-∞} = area under the concentration time curve from time 0 to infinity; C₀ = initial drug concentration; CL = clearance; MRT_{0-∞} = mean residence time, 0 to infinity; t½ = half life.
^{a} Single values based on sparse sampling across multiple animals.

After a single bolus IV injection of Compound 1 administered at 10 mg/kg in mice, the PK parameters were calculated as follows: for the time interval between 1 and 8 hours, the half life (t½) was 1.4 hours, the initial time zero concentration (Co) back extrapolated to 88 μg/mL, the area under the concentration time curve from time 0 to infinity (AUC_{0-∞}) was 18.5 hr*μg/mL, clearance (CL) was 541 mL/hr/kg, mean residence time (MRT) was 0.6 hours, and the volume of distribution at steady state (Vₛₛ) was 340 mL/kg.

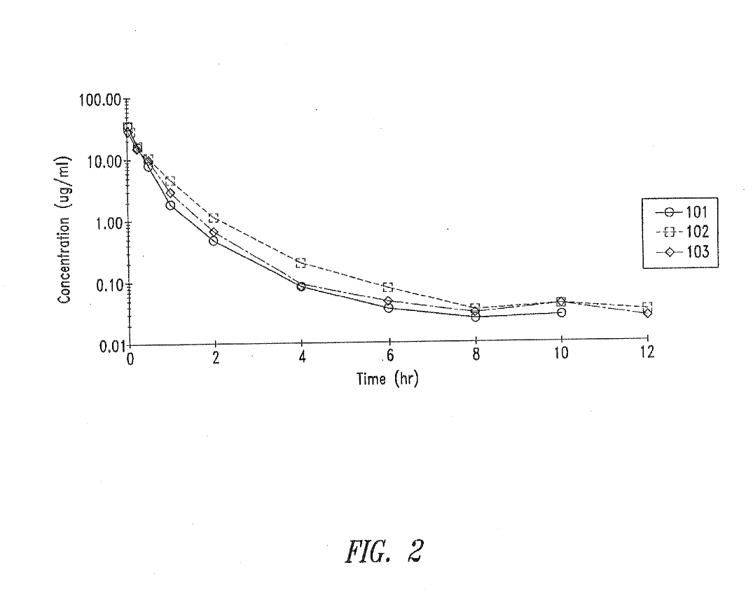

The plasma concentrations of Compound 1 observed in three rats (Rat 101, Rat 102 and Rat 103) at each timepoint up to 12 hours following administration are shown in FIG. 2. The noncompartmental PK parameters estimated for Compound 1 administered at 10 mg/kg as a bolus IV injection in rats are shown in Table 2.

TABLE 2
PK Parameter Estimates for a Single Bolus IV Injection
of Compound 1 Administered at 10 mg/kg in Rats

| Parameter | Rat 101 | Rat 102 | Rat 103 | Mean ± SD |
|---|---|---|---|---|
| t_{1/2} (hr) | 0.95 | 0.93 | 0.93 | 0.94 ± 0.01 |
| C₀ (μg/mL) | 41.7 | 42.2 | 28.8 | 37.5 ± 7.6 |
| CL (mL/hr/kg) | 725 | 541 | 678 | 648 ± 96 |
| AUC_{0-∞} (hr*μg /mL) | 13.8 | 18.5 | 14.8 | 15.7 ± 2.5 |
| MRT_{0-∞} (hr) | 0.6 | 0.8 | 0.7 | 0.7 ± 0.1 |
| Vₛₛ (mL/kg) | 403 | 434 | 473 | 437 ± 35 |

Following a single bolus IV injection of Compound 1 administered at 10 mg/kg in rats, the mean noncompartmental PK parameter estimations were as follows: for the time interval between 1 and 8 hours, the half life was 0.94 hours, the initial time zero concentration (back extrapolated) was 37.5 μg/mL, the AUC was 15.7 hr*μg/mL, the clearance was 648 mL/hr/kg, mean residence time was 0.7 hours, and the volume of distribution at steady state was 437 mL/kg.

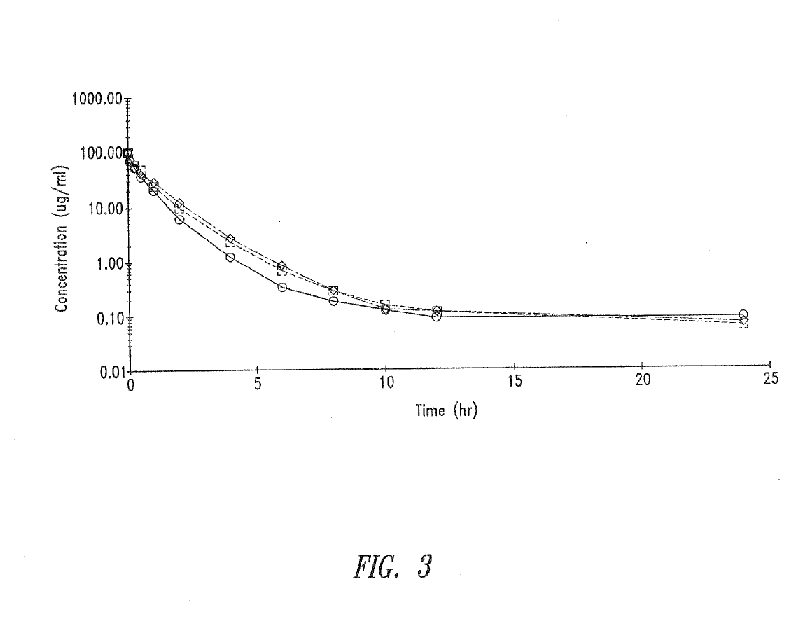

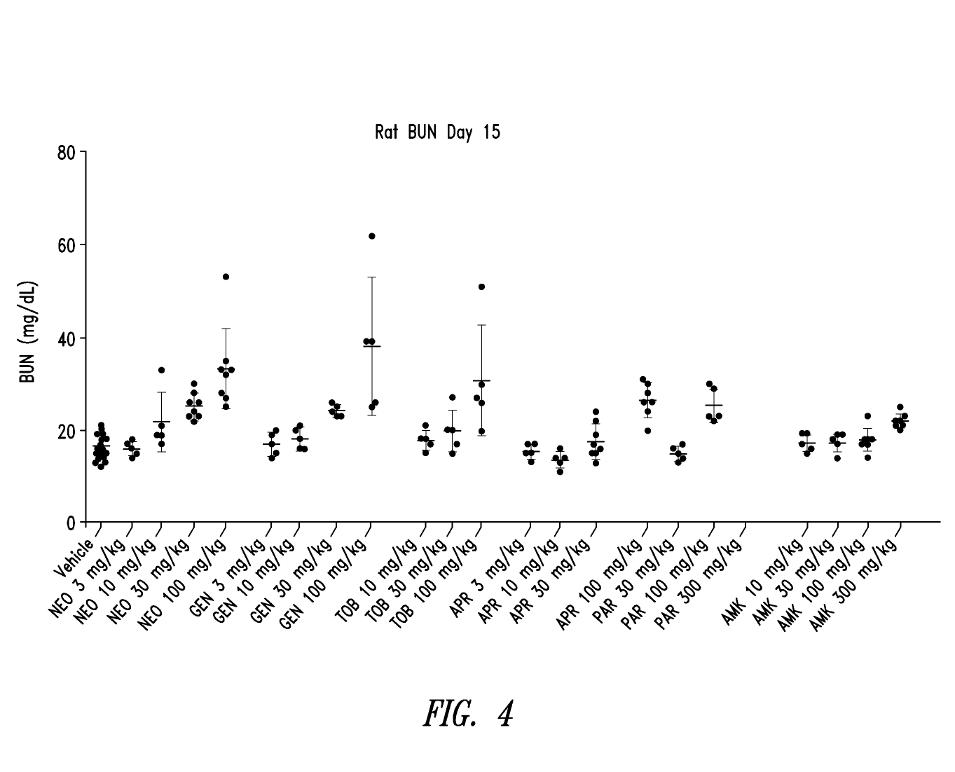

The plasma concentrations of Compound 1 observed in three beagle dogs (Dog 601, Dog 602 and Dog 603) at each timepoint up to 24 hours following administration are shown in FIG. 3. The noncompartmental PK parameters estimated for Compound 1 administered at 10 mg/kg as a bolus IV injection in each dog and the mean value of each parameter are shown in Table 3. The urine concentrations of Compound 1 observed in dogs at each timepoint up to 24 hours are shown in Table 4.

TABLE 3
PK Parameter Estimates of Compound 1 Administered
as a Single Dose Bolus IV Injection in Beagle Dogs

| Parameter | Dog 601 | Dog 602 | Dog 603 | Mean ± SD |
|---|---|---|---|---|
| HL Lambda Z | 1.18 | 1.24 | 1.23 | 1.22 ± 0.03 |
| (hr) | | | | |
| C₀ (μg/mL) | 134 | 119 | 106 | 120 ± 14 |
| CL (mL/hr/kg) | 144 | 113 | 111 | 123 ± 19 |
| AUC_{0-∞} | 69.4 | 88.7 | 89.7 | 82.6 ± 11.5 |
| (hr*μg/mL) | | | | |
| MRT_{0-∞} (hr) | 1.26 | 1.32 | 1.45 | 1.34 ± 0.10 |
| Vₛₛ (mL) | 181 | 149 | 162 | 164 ± 16 |

TABLE 4
Urine Concentration Analysis of Compound 1 Administered
as a Single Dose Bolus IV Injection in Beagle Dogs

| | | | Urine | Cmpd 1 | Cmpd 1 |
|---|---|---|---|---|---|
| | | Cmpd 1 | weight | recovery | recovery |
| Dog ID | Time | (ug/ml) | (g)^{a} | (ug) | (mg) |
| 6001 | Predose | 0 | 178 | 0 | 0 |
| | 6 hr | 1000 | 138 | 138140 | 138 |
| | 12 hr | 47 | 33 | 1555 | 2 |
| | 24 hr | 24 | 66 | 1557 | 2 |
| | Total | 1070 | 415 | 141252 | 141 |
| 6002 | Predose | 0 | 49 | 0 | 0 |
| | 6 hr | 505 | 334 | 168513 | 169 |
| | 12 hr | NS | NS | NS | NS |
| | 24 hr | 68 | 243 | 16487 | 16 |
| | Total | 573 | 626 | 185000 | 185 |
| 6003 | Predose | 0 | 276 | 0 | 0 |
| | 6 hr | 1180 | 54 | 63236 | 63 |
| | 12 hr | 100 | 43 | 4250 | 4 |
| | 24 hr | 44 | 99 | 4314 | 4 |
| | Total | 1324 | 470 | 71800 | 72 |

Final Cmpd. Recovery

| | | | Total | | |
|---|---|---|---|---|---|
| | | Dog | Cmpd 1 | Cmpd 1 | % of |
| | Dose | weight | dosed | recovered in | Cmpd 1 |
| | (mg/kg) | (kg) | (mg) | urine (mg) | recovered |
| 6001 | 10 | 10.52 | 105.2 | 141 | 134 |
| 6002 | 10 | 13.72 | 137.2 | 185 | 135 |
| 6003 | 10 | 8.25 | 82.5 | 72 | 87 |
| Mean (±SD) | | | | | 119 ± 22 |

NS = No sample
*Assume 1 gram equals 1 ml

The PK profile in dogs obtained after plasma samples were measured for the presence of Compound 1 by LC-MS/MS, and quantitative results were analyzed by WinNonLin for PK parameter estimates, was as follows for mean values: for the time interval between 0.083 and 12 hours, the half life was 1.22 hours, the initial time zero concentration (back extrapolated) was 120 μg/mL, AUC was 83 hr*μg/mL, clearance was 123 mL/hr/kg, mean residence time was 1.34 hours, and the volume of distribution at steady state was 164 mL/kg. A high percentage of Compound 1 was recovered in the urine after a single 10-mg/kg IV dose, as revealed by the mean Compound 1 urinary recovery from three dogs after the first 24 hours of 119%±22%.

The PK profile of Compound 1 after intravenous dosing was similar in mice, rats, and dogs. In rats, Cₘₐₓ and exposure (AUC) were dose linear to 75 mg/kg. Compound 1 was renally cleared rapidly in both rats and dogs (t½˜1 hr). Compound 1 distributed to rat kidneys. The volume of distribution closely matched extracellular fluid volumes. Compound 1 had low plasma protein binding (<20%). Compound 1 was stable when exposed to plasma, liver microsomes and hepatocytes and did not inhibit the 5 major human cytochrome P450 isoforms (CYPs) in vitro.

These studies demonstrate that Compound 1 is metabolically stable and unlikely to show drug-drug interactions. The short elimination half-life of Compound 1 observed in preclinical species predicts a short half-life in humans (˜1 hr). The PK profile of Compound 1 supports use of high doses in short infusions, administered once-daily to achieve high Cₘₐₓ and AUC, which are important for both efficacy and safety.

### Biological Example 2

### Quantitative Comparison of Aminoglycoside Nephrotoxicity in Rats

Aminoglycosides (AGs) are a well-known class of antibiotics with an established record of efficacy. However, their use has been limited due to concerns of nephrotoxicity. To support the development of new AGs and dosing regimens, a rat toxicity model was developed to effectively quantitate AG nephrotoxic potential. The model integrates extensive past research on AG nephrotoxicity and allows for effective screening of novel AGs and dosing regimens associated with reduced nephrotoxicity.

This rat nephrotoxicity study design used 14 days of once-daily dosing of aminoglycosides in adult Sprague-Dawley rats. Rats were allowed full access to food and water, and aminoglycosides were dosed subcutaneously at 1 mL/kg dosing volume, formulated in water. Nephrotoxicity was assessed by monitoring changes in serum markers of glomerular filtration rate (GFR), namely blood urea nitrogen (BUN) and serum creatinine. Microscopic examination of kidney slices after fixation and hematoxylin and eosin (H&E) staining was also utilized, scoring for tubular dilation, cellular casts, interstitial inflammation, and regenerative changes to the tubules.

This rat model provided a consistent measure of AG nephrotoxicity, as evidenced by the reliable dose-response of serum creatinine changes for gentamicin across a number of independent studies (no change at 10 mg/kg, mild elevation at 30 mg/kg, and >2× elevation/mortality at 100 mg/kg).

Neomycin (NEO), gentamicin (GEN), apramycin (APR), tobramycin (TOB), paromomycin (PAR), and amikacin (AMK) were evaluated in the rat model to determine their relative nephrotoxicity. As shown in FIG. 4, each aminoglycoside showed a dose-response of effect on rat kidney function (as measured by BUN) after 14 days of once-daily dosing. Kidney histopathology of rats treated with each aminoglycosides showed a similar pattern of changes (Table 5), including proximal tubular dilation, cellular casts in the tubular lumen (indicative of tubular cell destruction), interstitial inflammation, and evidence of tubular cell regeneration. However, the extent of functional impairment and intensity of histopathology changes, and the doses at which these changes occur, were different between aminoglycosides. AG-induced kidney changes were detected by H&E staining at doses many multiples below those that cause a glomerular filtration rate (GFR) functional deficit (e.g., 30× for gentamicin), illustrating the relative sensitivity of kidney histopathology to detect aminoglycoside-induced changes, and the capacity of the kidney to respond effectively to those changes without detectable effects on kidney function.

TABLE 5
Rat kidney histopathology analysis after
14 days of once-daily dosing of AGs

| | Dose | Average Histopathology Scores (0 to 5 Scale) | | | |
|---|---|---|---|---|---|
| | (mg/ | | | Inflamma- | Regenera- |
| AG | kg) | Dilation | Casts | tion | tion |
| Vehicle | | 0.2 +/− 0.4 | 0 +/− 0.2 | 0.3 +/− 0.5 | 0.3 +/− 0.6 |
| NEO | 3 | 0 +/− 0 | 0 +/− 0 | 0.8 +/− 0.4 | 0.4 +/− 0.5 |
| | 10 | 0.2 +/− 0.4 | 0.2 +/− 0.4 | 1.2 +/− 0.4 | 1 +/− 0 |
| | 30 | 1.2 +/− 0.4 | 1.6 +/− 0.5 | 2.4 +/− 0.5 | 2.4 +/− 0.5 |
| | 100 | 2 +/− 1.4 | 2 +/− 0.7 | 2.6 +/− 0.5 | 2.6 +/− 0.5 |
| GEN | 30 | 0 +/− 0 | 0 +/− 0 | 1.4 +/− 0.5 | 0.8 +/− 1.1 |
| | 10 | 0 +/− 0 | 0 +/− 0 | 1 +/− 0.7 | 0.4 +/− 0.5 |
| | 30 | 3 +/− 0 | 1.6 +/− 0.9 | 3 +/− 0.7 | 3.4 +/− 0.5 |
| | 100 | 2.6 +/− 0.5 | 2.8 +/− 0.4 | 3.6 +/− 0.9 | 4.6 +/− 0.5 |
| TOB | 10 | 0.4 +/− 0.9 | 0 +/− 0 | 0.4 +/− 0.9 | 1 +/− 1.4 |
| | 30 | 1.6 +/− 1.1 | 0.8 +/− 0.4 | 1.8 +/− 0.8 | 2.2 +/− 0.8 |
| | 100 | 1.6 +/− 1.1 | 1.8 +/− 0.8 | 2.4 +/− 0.9 | 3 +/− 0.7 |
| APR | 3 | 0 +/− 0 | 0 +/− 0 | 0.4 +/− 0.5 | 0 +/− 0 |
| | 10 | 0 +/− 0 | 0 +/− 0 | 0.4 +/− 0.5 | 0.6 +/− 1.3 |
| | 30 | 0 +/− 0 | 0 +/− 0 | 0.6 +/− 0.5 | 0.6 +/− 0.5 |
| | 100 | 0.6 +/− 0.5 | 1.4 +/− 0.5 | 2.4 +/− 0.5 | 2.4 +/− 0.5 |
| PAR | 30 | 1 +/− 1 | 0 +/− 0 | 1.4 +/− 0.9 | 1.4 +/− 0.5 |
| | 100 | 3.2 +/− 0.4 | 2.6 +/− 0.5 | 3 +/− 0 | 4 +/− 0 |
| | 300 | 0 +/− 0 | 0 +/− 0 | 0 +/− 0 | 0 +/− 0 |
| AMK | 10 | 0.2 +/− 0.4 | 0 +/− 0 | 0.4 +/− 0.5 | 0.2 +/− 0.4 |
| | 30 | 0 +/− 0 | 0 +/− 0 | 0.4 +/− 0.5 | 0.2 +/− 0.4 |
| | 100 | 0.2 +/− 0.4 | 0 +/− 0 | 0.6 +/− 0.5 | 0.6 +/− 0.5 |
| | 300 | 1.2 +/− 0.8 | 0 +/− 0 | 1.2 +/− 0.4 | 1.4 +/− 0.5 |

Each AG tested exhibited a dose-response effect on kidney function (BUN), and a similar pattern of kidney histopathology changes was observed for each AG. However, functional impairment and microscopic kidney changes occurred at different dose levels for the different AGs, revealing quantitatively different nephrotoxic potentials.

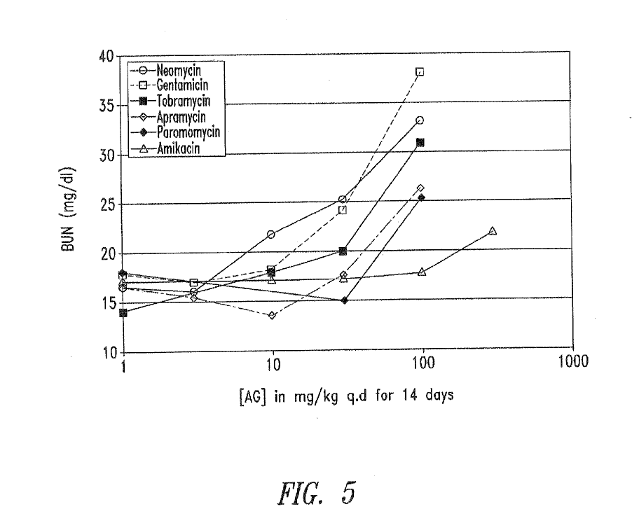

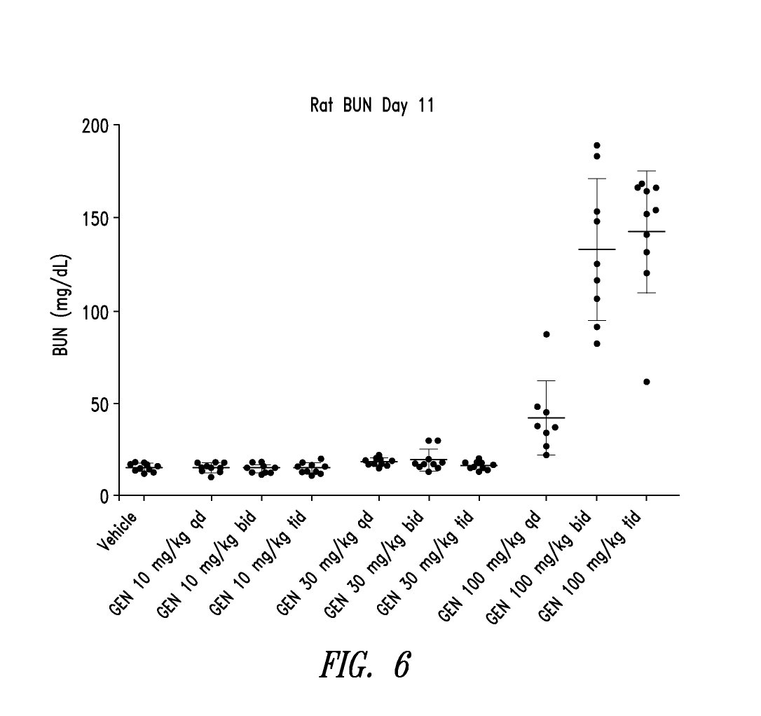

The nephrotoxic ranking of the aminoglycosides tested in this rat model correlated well with their relative clinical nephrotoxicity, where clinical data was available. FIG. 5 shows an overlay of the dose-response for BUN elevation by each AG. The apparent relative nephrotoxicity of aminoglycosides in this rat model is: Neomycin≈Gentamicin>Tobramycin>Apramycin≈Paromomycin>Amikacin These results are in general agreement with prior investigations in rats.

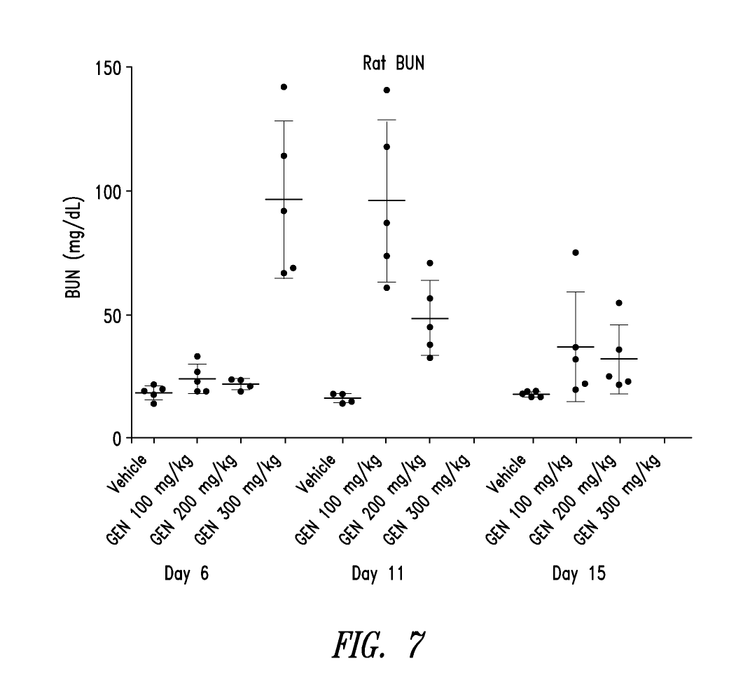

Consistent with prior work suggesting that kidney uptake of AGs is a saturable process, once-daily dosing (qd) of gentamicin was significantly less toxic than twice- or three-times daily dosing of the same total daily dose. As shown in FIG. 6, delivering 100 mg/kg/day as 2 or 3 separate doses per day (bid or tid, respectively) led to a significantly worse impairment of kidney function (p<0.0001) as measured by BUN. 100 mg/kg/day gentamicin resulted in less impairment of kidney function when administered once-daily instead of in 2 or 3 separate doses per day.

Supporting the model that AG nephrotoxicity is correlated to the total duration of treatment, it was demonstrated that limiting the duration of dosing to 5 days allowed for doubling the dose of gentamicin without a significant increase in toxicity as compared to 14 days of dosing. FIG. 7 shows the BUN from rats dosed once-daily for 5 days with 100, 200, or 300 mg/kg/day gentamicin. After 5 days of administration, the nephrotoxicity of 100 and 200 mg/kg/day dose levels were indistinguishable from each other and from 100 mg/kg/day given longer (14 days). A tripling of the dose from 100 to 300 mg/kg/day led to a measurable increase in kidney function impairment on day 6 and subsequent mortality. These results demonstrate that limiting dosing duration to 5 days allows for doubling of dose without an increase in toxicity.

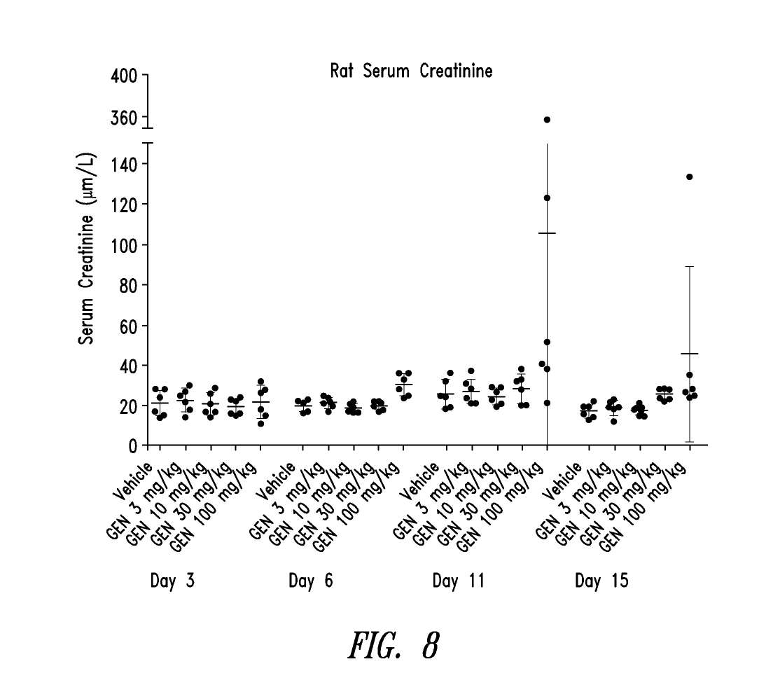

The time course of gentamicin nephrotoxicity in rats was examined in detail. FIG. 8 shows the progression of serum creatinine changes after once-daily dosing of gentamicin for 14 days at levels up to 100 mg/kg/day. No changes in kidney function were observed at any dose level prior to day 6. At 100 mg/kg/day, serum creatinine began to rise slightly on day 6, peaked around day 11, and normalized by day 15. At the lower dose of 30 mg/kg/day, no rise in serum creatinine was observed until day 15, when a mild impairment of GFR was measurable. Doses lower than 30 mg/kg/day did not impair GFR over the 14 days of dosing.

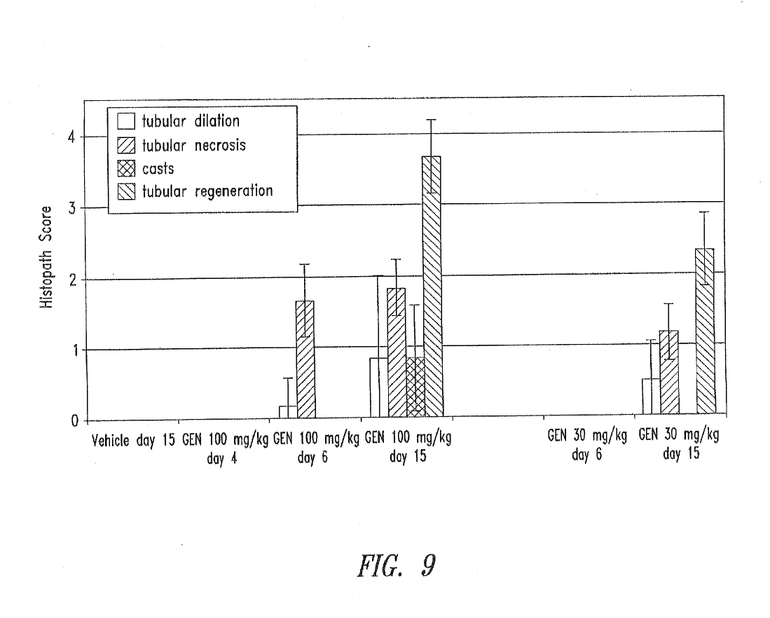

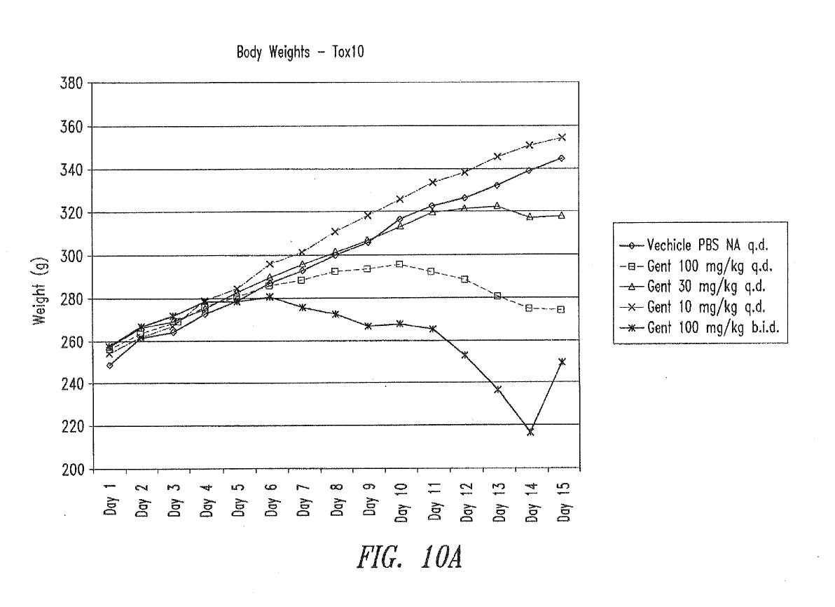

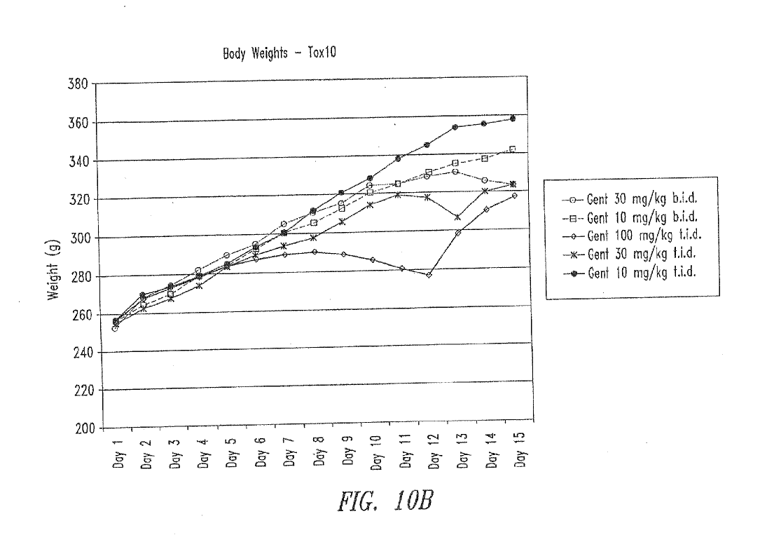

FIG. 9 presents the kidney histopathology results from the same study design. Prior to day 6, not only was no kidney functional impairment evident (FIG. 9), but no microscopic changes were evident in the kidneys at the high dose level of 100 mg/kg/day. At that high dose, signs of kidney necrosis appeared after 5 days of dosing, and both on-going damage and regeneration of kidney tubule epithelium was observed after 14 days of dosing. At the lower dose level of 30 mg/kg/day, no microscopic changes were seen even after 5 days of dosing, but signs of damage and regeneration were evident after 14 days of dosing, consistent with serum creatinine changes (FIG. 9). No changes in kidney function were observed at any dose level prior to day 6.

To summarize the results of this study, at 100 mg/kg/day, creatinine peaked at day 11 and normalized by day 15. At 30 mg/kg/day, creatinine rose slightly on day 15, whereas doses <30 mg/kg/day did not impair GFR over the 14 days of dosing. At 100 mg/kg/day, no microscopic changes in kidneys were observed prior to day 6. At 30 mg/kg/day, no microscopic changes in kidneys were observed prior to day 15. Signs of tissue regeneration (recovery) were observed by day 15.

This 14-day rat model provides a consistent and reliable evaluation of nephrotoxicity of AGs, and also allows screening of new AG derivatives to guide selection of less toxic neoglycosides for clinical development. In addition, it allows exploration of rationales for dosing of AGs to minimize toxicity.

Consistent with the model that kidney uptake of AGs is a saturable process, once-daily dosing of gentamicin was significantly less toxic than twice- or three-times daily dosing of the same total daily dose. Also, supporting the model that AG nephrotoxicity is correlated to the total duration of treatment, it was shown that limiting the duration of dosing to 5 days allowed for doubling the dose of gentamicin without a significant increase in toxicity compared with 14 days of dosing. Thus, the studies described herein provide evidence that less frequent (once-daily) dosing of AGs leads to reduced toxicity and that higher dose, shorter course therapy of AGs allowed effective treatment without an increase in toxicity.

### Biological Example 3

### Nephrotoxicity of Gentamicin Administered Fourteen Days or Five Days

The nephrotoxicity of the aminoglycosides (AGs) gentimicin (Gent) and tobramycin (Tobr) following administration to Sprague-Dawley rats at various dosage levels and dosage regimes was compared using the rat nephrotoxicity model described in Biological Example 2. These studies demonstrated that more frequent administration of gentamicin resulted in increased toxicity as compared to less frequent administration of the same dosage. In addition, they demonstrated that dosing regimens of short duration (e.g., 5 days) resulted in decreased toxicity as compared to dosing regimens of longer duration (e.g., 14 days) of the same dosage. These studies support the use of high dosages of aminoglycosides for short durations of time and/or less frequent administration.

The AGs were administered to the rats according to the dose levels depicted in Table 6. The animals of Groups 11-13 were housed until day 15 even though their dosing ended on day 5, such that all animals were necropsied on the same day. Blood was collected in EDTA at days 0, 6, 11, and 15. 750 uL blood samples were collected and aliquoted into a lithium heparin tube and an EDTA tube. Clinical chemistry analysis was performed using the Idexx VETTEST 8008 system. Kidney samples were embedded in paraffin, sectioned, stained with hematoxylin and eosin and examined microscopically.

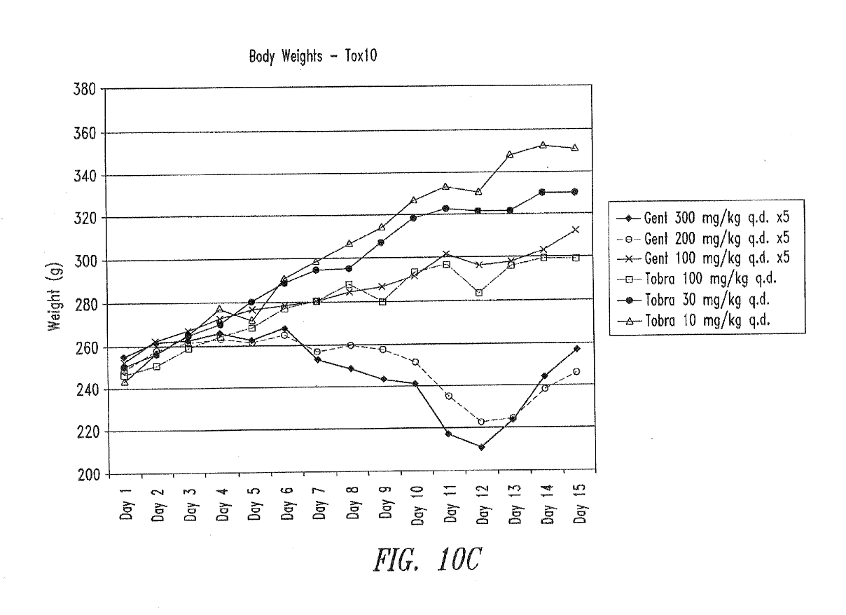

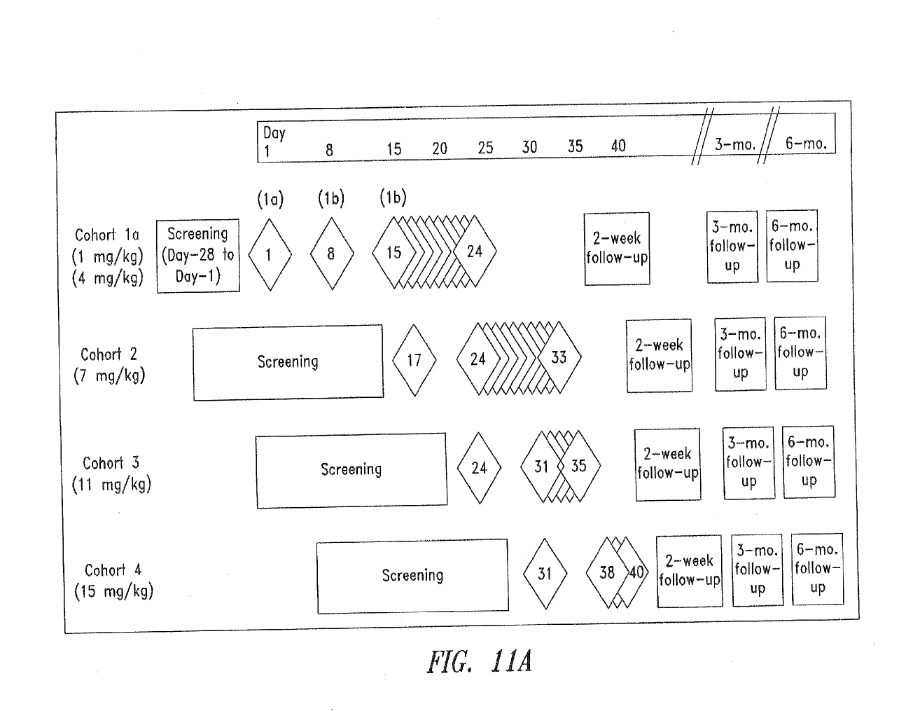

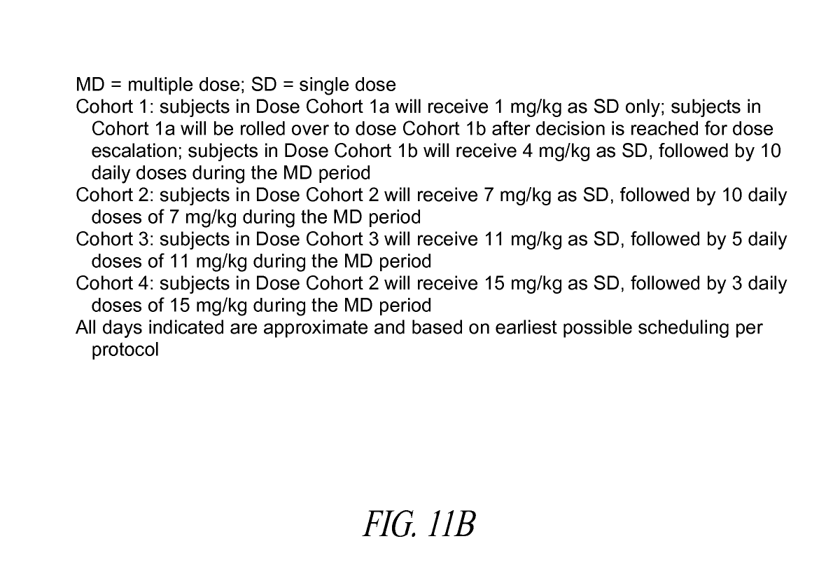

The results of these studies are presented in Tables 7-13. A graphical depiction of body weight is provided in FIG. 10.

TABLE 6
Group designation and dosage levels

| | | | | | | | Dose | Dose | |
|---|---|---|---|---|---|---|---|---|---|
| | Number of | Test | Dose Levels | | Dose | Dosing | volume | Conc. | |
| Group | Animals | Article | (mg/kg) | | regimen | Duration | (ml/kg) | (mg/mL) | Animal ID Numbers |
| 1 | 5 | Vehicle | NA | | q.d. | 14 d | 1 ml/kg | NA | 101, 102, 103, 104, 105 |
| | | PBS | | | | | | | |
| 2 | 5 | Gent | 100 | mg/kg | q.d. | 14 d | 1 ml/kg | 100 mg/ml | 201, 202, 203, 204, 205 |
| 3 | 5 | Gent | 30 | mg/kg | q.d. | 14 d | 1 ml/kg | 30 | 301, 302, 303, 304, 305 |
| 4 | 5 | Gent | 10 | mg/kg | q.d. | 14 d | 1 ml/kg | 10 | 401, 402, 403, 404, 405 |
| 5 | 5 | Gent | 100 | mg/kg | b.i.d. | 14 d | 1 ml/kg | 50 mg/ml | 501, 502, 503, 504, 505 |
| 6 | 5 | Gent | 30 | mg/kg | b.i.d. | 14 d | 1 ml/kg | 15 | 601, 602, 603, 604, 605 |
| 7 | 5 | Gent | 10 | mg/kg | b.i.d. | 14 d | 1 ml/kg | 5 | 701, 702, 703, 704, 705 |
| 8 | 5 | Gent | 100 | mg/kg | t.i.d. | 14 d | 1 ml/kg | 33 mg/ml | 801, 802, 803, 804, 805 |
| 9 | 5 | Gent | 30 | mg/kg | t.i.d. | 14 d | 1 ml/kg | 10 | 901, 902, 903, 904, 905 |
| 10 | 5 | Gent | 10 | mg/kg | t.i.d. | 14 d | 1 ml/kg | 3.3 | 1001, 1002, 1003, 1004, 1005 |
| 11 | 5 | Gent | 300 | mg/kg | q.d. | 5 d | 1 ml/kg | 300 mg/ml | 1101, 1102, 1103, 1104, 1105 |
| 12 | 5 | Gent | 200 | mg/kg | q.d. | 5 d | 1 ml/kg | 200 | 1201, 1202, 1203, 1204, 1205 |
| 13 | 5 | Gent | 100 | mg/kg | q.d. | 5 d | 1 ml/kg | 100 | 1301, 1302, 1303, 1304, 1305 |
| 14 | 5 | Tobra | 100 | mg/kg | q.d. | 14 d | 1 ml/kg | 100 mg/ml | 1401, 1402, 1403, 1404, 1405 |
| 15 | 5 | Tobra | 30 | mg/kg | q.d. | 14 d | 1 ml/kg | 30 | 1501, 1502, 1503, 1504, 1505 |
| 16 | 5 | Tobra | 10 | mg/kg | q.d. | 14 d | 1 ml/kg | 10 | 1601, 1602, 1603, 1604, 1605 |

TABLE 7
Group body weight

| | Dose | | | | | | | | | | | | | | | | | |
|---|---|---|---|---|---|---|---|---|---|---|---|---|---|---|---|---|---|---|
| Test | Levels | | Dose | | Day | Day | Day | Day | Day | Day | Day | Day | Day | Day | Day | Day | Day | Day |
| Article | (mg/kg) | | regimen | Group | 1 | 2 | 3 | 4 | 5 | 6 | 7 | 8 | 9 | 10 | 11 | 12 | 13 | 14 |
| Vehicle | NA | | q.d. | 1 | 248.4 | 261.2 | 263.6 | 272.4 | 278.8 | 286.4 | 293 | 300.2 | 306 | 316.4 | 322.8 | 326.4 | 331.4 | 338.6 |
| PBS | | | | | | | | | | | | | | | | | | |
| Gent | 100 | mg/kg | q.d. | 2 | 254 | 262 | 267 | 276 | 280 | 286 | 288 | 292 | 293 | 295 | 292 | 288 | 280 | 275 |
| Gent | 30 | mg/kg | q.d. | 3 | 257 | 266 | 269 | 275 | 283 | 290 | 296 | 301 | 307 | 314 | 320 | 322 | 322 | 318 |
| Gent | 10 | mg/kg | q.d. | 4 | 254 | 261 | 267 | 279 | 284 | 296 | 301 | 311 | 318 | 326 | 334 | 338 | 345 | 351 |
| Gent | 100 | mg/kg | b.i.d. | 5 | 257 | 267 | 272 | 278 | 278 | 280 | 275 | 272 | 266 | 268 | 265 | 252 | 236 | 217 |
| Gent | 30 | mg/kg | b.i.d. | 6 | 252 | 268 | 274 | 282 | 289 | 294 | 305 | 310 | 315 | 324 | 324 | 328 | 331 | 326 |
| Gent | 10 | mg/kg | b.i.d. | 7 | 256 | 264 | 270 | 279 | 283 | 292 | 300 | 306 | 313 | 321 | 325 | 330 | 335 | 337 |
| Gent | 100 | mg/kg | t.i.d. | 8 | 256 | 267 | 272 | 278 | 284 | 286 | 289 | 290 | 288 | 285 | 280 | 277 | 298 | 310 |
| Gent | 30 | mg/kg | t.i.d. | 9 | 253.8 | 261.4 | 268.8 | 273.6 | 283 | 289 | 293 | 297.4 | 305.2 | 313.6 | 318.8 | 317.2 | 306.6 | 319.8 |
| Gent | 10 | mg/kg | t.i.d. | 10 | 255.4 | 269.6 | 273.2 | 278.6 | 284.6 | 293.2 | 300.6 | 311.4 | 320.8 | 328.2 | 338.2 | 345 | 354.4 | 355.6 |
| Gent | 300 | mg/kg | q.d. x5 | 11 | 255 | 261 | 262 | 266 | 262 | 268 | 253 | 248 | 243 | 241 | 217 | 211 | 223 | 244 |
| Gent | 200 | mg/kg | q.d. x5 | 12 | 248.8 | 258 | 261.4 | 263.4 | 262.2 | 264.8 | 256.8 | 259.8 | 257.6 | 252 | 236 | 223 | 224 | 239 |
| Gent | 100 | mg/kg | q.d. x5 | 13 | 252 | 262 | 266 | 272 | 277 | 278 | 280 | 284 | 287 | 291 | 301 | 296 | 298 | 303 |
| Tobra | 100 | mg/kg | q.d. | 14 | 246 | 251 | 259 | 264 | 268 | 277 | 281 | 287 | 280 | 293 | 296 | 283 | 296 | 299 |
| Tobra | 30 | mg/kg | q.d. | 15 | 250 | 256 | 264 | 270 | 280 | 288 | 294 | 295 | 307 | 318 | 323 | 321 | 321 | 330 |
| Tobra | 10 | mg/kg | q.d. | 16 | 243 | 256 | 264 | 277 | 271 | 291 | 299 | 307 | 315 | 327 | 333 | 330 | 347 | 352 |

TABLE 8
Clinical chemistries results

| Test Code | Test Name | Reference Range* | Units |
|---|---|---|---|
| ALB | Albumin | 3.8-4.8 | g/dl |
| ALKP | Alkaline Phophatase | 16-302 | U/L |
| ALT | Alanine aminotranferase | 20-61 | U/L |
| AMYL | Amylase | 326-2246 | U/L |
| AST | Aspartate aminotranferase | 39-111 | U/L |
| BUN | Blood urea nitrogen | 9-21 | mg/dl |
| Ca | Calcium | 5.3-11.6 | mg/dl |
| CHOL | Cholesterol | 20-92 | mg/dl |
| CREA | Creatinine | 0.1-0.6 | mg/dl |
| GLU | Glucose | 50-135 | mg/dl |
| TBIL | Total bilirubin | 0.1-0.7 | mg/dl |
| TP | Total protein | 5.3-6.9 | g/dl |
| GLOB | Globulin (calculated) | 1.5-2.8 | g/dl |

TABLE 9
Group results day 0

| Dose | | | | | | | | | | | | | | | | |
|---|---|---|---|---|---|---|---|---|---|---|---|---|---|---|---|---|
| Levels | | Dose | | | | | | | | | | | | | | |
| (mg/kg) | | regimen | Group | BUN | GLU | ALP | T-PRO | ALT | Cre | CA | ALB | AST | GLOB | TBIL | CHOL | AMYL |
| NA | | q.d. | 1 | 15.6 | 161.6 | 560.8 | 5.8 | 33 | 0.5 | 10.7 | 3.7 | 57 | 2.1 | 0.1 | 76 | 1510 |
| 100 | mg/kg | q.d. | 2 | 16.4 | 141 | 601 | 5.7 | 25 | 0.5 | 10.9 | 3.9 | 61 | 1.8 | 0.1 | 75 | 1521 |
| 30 | mg/kg | q.d. | 3 | 14.6 | 144 | 552 | 5.9 | 22 | 0.4 | 10.6 | 4.0 | 72 | 1.9 | 0.1 | 71 | 1631 |
| 10 | mg/kg | q.d. | 4 | 16.4 | 138 | 548 | 5.7 | 18 | 0.5 | 10.9 | 4.0 | 74 | 1.8 | 0.1 | 71 | 1663 |
| 100 | mg/kg | b.i.d. | 5 | 15.6 | 154 | 537 | 6.1 | 31 | 0.5 | 10.8 | 4.2 | 63 | 1.9 | 0.1 | 74 | 1509 |
| 30 | mg/kg | b.i.d. | 6 | 18.2 | 135 | 586 | 6.0 | 35 | 0.5 | 10.9 | 4.0 | 67 | 1.9 | 0.1 | 78 | 1509 |
| 10 | mg/kg | b.i.d. | 7 | 16.0 | 142 | 544 | 5.8 | 40 | 0.5 | 10.7 | 3.9 | 63 | 2.0 | 0.1 | 67 | 1633 |
| 100 | mg/kg | t.i.d. | 8 | 16.4 | 143 | 537 | 5.9 | 34 | 0.5 | 10.6 | 3.9 | 60 | 1.8 | 0.1 | 73 | 1656 |
| 30 | mg/kg | t.i.d. | 9 | 17.8 | 143 | 604 | 5.9 | 36 | 0.5 | 10.7 | 4.0 | 58 | 1.9 | 0.1 | 81 | 1668 |
| 10 | mg/kg | t.i.d. | 10 | 15.2 | 153 | 557 | 5.7 | 33 | 0.4 | 10.8 | 4.0 | 68 | 1.9 | 0.1 | 75 | 1624 |
| 300 | mg/kg | q.d. x5 | 11 | 16.8 | 159 | 570 | 5.8 | 37 | 0.6 | 10.5 | 4.0 | 63 | 1.9 | 0.1 | 77 | 1606 |
| 200 | mg/kg | q.d. x5 | 12 | 16.8 | 155 | 583 | 5.8 | 29 | 0.6 | 10.9 | 3.9 | 61 | 2.0 | 0.1 | 79 | 1526 |
| 100 | mg/kg | q.d. x5 | 13 | 17.4 | 174 | 568 | 5.8 | 29 | 0.5 | 11.0 | 3.9 | 71 | 1.9 | 0.1 | 79 | 1583 |
| 100 | mg/kg | q.d. | 14 | 17.0 | 155 | 587 | 6.0 | 35 | 0.5 | 10.6 | 4.0 | 69 | 2.0 | 0.1 | 73 | 1403 |
| 30 | mg/kg | q.d. | 15 | 16.6 | 135 | 540 | 5.8 | 36 | 0.5 | 10.9 | 3.9 | 58 | 1.9 | 0.1 | 74 | 1547 |
| 10 | mg/kg | q.d. | 16 | 16.8 | 146 | 559 | 6.0 | 28 | 0.6 | 10.5 | 3.9 | 65 | 2.1 | 0.1 | 80 | 1627 |

TABLE 10
Group results day 6

| | Dose | | | | | | | | | | | | | | | | |
|---|---|---|---|---|---|---|---|---|---|---|---|---|---|---|---|---|---|
| Test | Levels | | Dose | | | | | | | | | | | | | | |
| Article | (mg/kg) | | regimen | Group | BUN | GLU | ALP | T-PRO | ALT | Cre | CA | ALB | AST | GLOB | TBIL | CHOL | AMYL |
| Vehicle | NA | | q.d. | 1 | 15 | 133 | 745 | 5.8 | 37 | 0.6 | 10.3 | 4.0 | 65 | 1.9 | 0.1 | 78 | 1785 |
| PBS | | | | | | | | | | | | | | | | | |
| Gent | 100 | mg/kg | q.d. | 2 | 17 | 146 | 619 | 6.1 | 28 | 0.6 | 10.5 | 4.2 | 67 | 1.9 | 0.1 | 90 | 1596 |
| Gent | 30 | mg/kg | q.d. | 3 | 16 | 142 | 641 | 5.9 | 35 | 0.6 | 9.9 | 4.1 | 67 | 1.8 | 0.1 | 87 | 1675 |
| Gent | 10 | mg/kg | q.d. | 4 | 15 | 154 | 807 | 5.9 | 33 | 0.6 | 10.3 | 4.1 | 73 | 1.7 | 0.1 | 77 | 1715 |
| Gent | 100 | mg/kg | b.i.d. | 5 | 16 | 148 | 635 | 6.0 | 31 | 0.9 | 10.5 | 4.2 | 95 | 1.8 | 0.2 | 88 | 1617 |
| Gent | 30 | mg/kg | b.i.d. | 6 | 13 | 163 | 571 | 5.8 | 31 | 0.7 | 10.7 | 4.1 | 63 | 1.7 | 0.1 | 82 | 1659 |
| Gent | 10 | mg/kg | b.i.d. | 7 | 11 | 149 | 732 | 5.8 | 38 | 0.7 | 10.9 | 4.1 | 72 | 1.7 | 0.1 | 70 | 1636 |
| Gent | 100 | mg/kg | t.i.d. | 8 | 15 | 158 | 604 | 6.1 | 33 | 0.8 | 10.9 | 4.2 | 74 | 1.9 | 0.1 | 81 | 1747 |
| Gent | 30 | mg/kg | t.i.d. | 9 | 14 | 146 | 654 | 6.1 | 29 | 0.8 | 10.7 | 4.0 | 60 | 2.1 | 0.1 | 70 | 1757 |
| Gent | 10 | mg/kg | t.i.d. | 10 | 13 | 147 | 747 | 5.7 | 24 | 0.6 | 10.8 | 4.2 | 58 | 1.5 | 0.1 | 78 | 1720 |
| Gent | 300 | mg/kg | q.d. x5 | 11 | 81 | 119 | 424 | 5.0 | 38 | 3.4 | 11.2 | 3.5 | 222 | 1.5 | 0.1 | 91 | 1609 |
| Gent | 200 | mg/kg | q.d. x5 | 12 | 54 | 130 | 462 | 5.8 | 59 | 2.1 | 12.04 | 3.8 | 281 | 2.0 | 0.1 | 86 | 1761 |
| Gent | 100 | mg/kg | q.d. x5 | 13 | 14 | 149 | 425 | 5.9 | 36 | 0.7 | 11.0 | 4.2 | 70 | 1.7 | 0.1 | 75 | 1640 |
| Tobra | 100 | mg/kg | q.d. | 14 | 19 | 148 | 596 | 5.6 | 36 | 0.7 | 10.8 | 4.0 | 75 | 1.6 | 0.1 | 82 | 1560 |
| Tobra | 30 | mg/kg | q.d. | 15 | 15 | 155 | 583 | 5.8 | 32 | 0.7 | 11.1 | 4.0 | 65 | 1.8 | 0.1 | 81 | 1658 |
| Tobra | 10 | mg/kg | q.d. | 16 | 16 | 154 | 670 | 5.9 | 29 | 0.8 | 11.1 | 4.0 | 65 | 1.8 | 0.1 | 78 | 1719 |

TABLE 11
Group results day 11

| | Dose | | | | | | | | | | | | | | | | |
|---|---|---|---|---|---|---|---|---|---|---|---|---|---|---|---|---|---|
| Test | Levels | | Dose | | | | | | | | | | | | | | |
| Article | (mg/kg) | | regimen | | BUN | GLU | ALP | T-PRO | ALT | Cre | CA | ALB | AST | GLOB | TBIL | CHOL | AMYL |
| Vehicle | NA | | q.d. | 1 | 14.6 | 136 | 640 | 6.1 | 44 | 0.7 | 11.0 | 4.0 | 80.8 | 2.0 | 0.1 | 75 | 1692 |
| PBS | | | | | | | | | | | | | | | | | |
| Gent | 100 | mg/kg | q.d. | 2 | 34 | 136 | 448 | 5.7 | 40 | 1.0 | 10.7 | 3.9 | 151 | 1.8 | 0.2 | 92 | 1808 |
| Gent | 30 | mg/kg | q.d. | 3 | 18 | 155 | 571 | 6.1 | 41 | 0.7 | 10.8 | 4.0 | 79 | 2.1 | 0.1 | 82 | 1762 |
| Gent | 10 | mg/kg | q.d. | 4 | 13 | 151 | 746 | 5.7 | 35 | 0.6 | 10.9 | 4.0 | 57 | 1.7 | 0.1 | 80 | 1658 |
| Gent | 100 | mg/kg | b.i.d. | 5 | 129 | 139 | 332 | 5.7 | 23 | 5.1 | 10.6 | 4.0 | 139 | 1.7 | 0.2 | 90 | 1669 |
| Gent | 30 | mg/kg | b.i.d. | 6 | 20 | 143 | 481 | 5.6 | 35 | 0.8 | 11.1 | 3.9 | 95 | 1.7 | 0.1 | 76 | 1670 |
| Gent | 10 | mg/kg | b.i.d. | 7 | 14 | 144.4 | 702 | 5.9 | 42 | 0.7 | 11.1 | 4.2 | 78 | 1.7 | 0.2 | 89 | 1623 |
| Gent | 100 | mg/kg | t.i.d. | 8 | 132 | 185 | 333 | 5.6 | 27 | 6.4 | 8.7 | 4.1 | 175 | 1.5 | 0.1 | 79 | 1709 |
| Gent | 30 | mg/kg | t.i.d. | 9 | 16.6 | 161.8 | 532 | 5.8 | 34 | 2.4 | 11.3 | 4.0 | 82 | 1.8 | 0.1 | 79 | 1599 |
| Gent | 10 | mg/kg | t.i.d. | 10 | 12.4 | 153 | 714 | 5.9 | 34 | 0.7 | 10.9 | 4.0 | 65 | 1.8 | 0.1 | 73 | 1707 |
| Gent | 300 | mg/kg | q.d. x5 | 11 | 158 | 158 | 256 | 7.2 | 12 | 7.1 | 11.1 | 4.5 | 51 | 2.7 | 0.2 | 99 | 2104 |
| Gent | 200 | mg/kg | q.d. x5 | 12 | 136 | 250 | 323 | 5.6 | 21 | 6.6 | 9.9 | 3.6 | 106 | 2.0 | 0.1 | 74 | 1711 |
| Gent | 100 | mg/kg | q.d. x5 | 13 | 20 | 143 | 490 | 5.8 | 35 | 0.8 | 10.7 | 4.0 | 70 | 1.8 | 0.1 | 78 | 1689 |
| Tobra | 100 | mg/kg | q.d. | 14 | 21 | 152 | 550 | 5.7 | 42 | 0.8 | 10.9 | 4 | 77 | 1.7 | 0.1 | 80 | 1765 |
| Tobra | 30 | mg/kg | q.d. | 15 | 18 | 150 | 568 | 5.6 | 43 | 0.7 | 11.2 | 4.0 | 63 | 1.6 | 0.1 | 75 | 1725 |
| Tobra | 10 | mg/kg | q.d. | 16 | 17 | 152 | 585 | 5.8 | 41 | 0.7 | 10.9 | 4.0 | 98 | 1.9 | 0.1 | 82 | 1606 |

TABLE 12
Group results day 15

| | Dose | | | | | | | | | | | | | | | | |
|---|---|---|---|---|---|---|---|---|---|---|---|---|---|---|---|---|---|
| Test | Levels | | Dose | | | | | | | | | | | | | | |
| Article | (mg/kg) | | regimen | | BUN | GLU | ALP | T-PRO | ALT | Cre | CA | ALB | AST | GLOB | TBIL | CHOL | AMYL |
| Vehicle | NA | | q.d. | 1 | 14 | 154 | 538 | 6.3 | 54 | 0.6 | 11.8 | 4.4 | 100 | 1.8 | 0.1 | 82 | 1895 |
| PBS | | | | | | | | | | | | | | | | | |
| Gent | 1.00 | mg/kg | q.d. | 2 | 38 | 137 | 337 | 6.1 | 38 | 1.0 | 11.5 | 4.5 | 130 | 1.6 | 0.1 | 76 | 1470 |
| Gent | 30 | mg/kg | q.d. | 3 | 20 | 144 | 334 | 6.0 | 59 | 0.7 | 11.4 | 4.4 | 126 | 1.6 | 0.1 | 82 | 1616 |
| Gent | 10 | mg/kg | q.d. | 4 | 13 | 170 | 575 | 5.9 | 76 | 0.5 | 12.0 | 4.4 | 107 | 1.5 | 0.1 | 83 | 1682 |
| Gent | 100 | mg/kg | b.i.d. | 5 | 53 | 156 | 294 | 5.6 | 35 | 1.1 | 11.5 | 4.5 | 98 | 1.1 | 0.1 | 101 | 1590 |
| Gent | 30 | mg/kg | b.i.d. | 6 | 26 | 141 | 375 | 5.9 | 43 | 0.7 | 11.3 | 4.4 | 129 | 1.5 | 0.1 | 80 | 1760 |
| Gent | 10 | mg/kg | b.i.d. | 7 | 13 | 169 | 522 | 6.1 | 64 | 0.5 | 11.7 | 4.4 | 124 | 1.7 | 0.1 | 80 | 1606 |
| Gent | 100 | mg/kg | t.i.d. | 8 | 71 | 249 | 372 | 5.6 | 49 | 3.5 | 9.7 | 4.2 | 170 | 1.4 | 0.1 | 106 | 1521 |
| Gent | 30 | mg/kg | t.i.d. | 9 | 23 | 154 | 427 | 5.8 | 41 | 0.8 | 12.0 | 4.3 | 92 | 1.4 | 0.1 | 85 | 1695 |
| Gent | 10 | mg/kg | t.i.d. | 10 | 14 | 169 | 548 | 5.8 | 37 | 0.5 | 11.7 | 4.3 | 88 | 1.5 | 0.1 | 83 | 1568 |
| Gent | 300 | mg/kg | q.d. x5 | 11 | 27 | 153 | 576 | 5.6 | 85 | 0.8 | 10.2 | 3.9 | 73 | 1.7 | 0.1 | 98 | 1497 |
| Gent | 200 | mg/kg | q.d. x5 | 12 | 41 | 188 | 431 | 5.1 | 189 | 0.8 | 10.7 | 4.0 | 349 | 1.1 | 0.2 | 104 | 1608 |
| Gent | 100 | mg/kg | q.d. x5 | 13 | 25 | 140 | 423 | 5.6 | 78 | 0.6 | 11.4 | 4.2 | 126 | 1.4 | 0.1 | 86 | 1721 |
| Tobra | 100 | mg/kg | q.d. | 14 | 31 | 151 | 459 | 6.1 | 52 | 0.8 | 11.7 | 4.2 | 123 | 1.9 | 0.1 | 76 | 1623 |
| Tobra | 30 | mg/kg | q.d. | 15 | 20 | 161 | 431 | 5.9 | 54 | 0.6 | 11.1 | 4.1 | 109 | 1.8 | 0.1 | 81 | 1583 |
| Tobra | 10 | mg/kg | q.d. | 16 | 18 | 176 | 533 | 5.9 | 41 | 0.5 | 11.3 | 4.12 | 99 | 1.8 | 0.1 | 77 | 1674 |

TABLE 13
Organ weights

| Rat | | | Kidney | Kidney | | | | |
|---|---|---|---|---|---|---|---|---|
| # | testes | Pancreas | Rt. | Lft | Liver | Heart | Lung | Brain |
| 1.1 | 3.37 | 0.88 | 1.37 | 1.41 | 15.27 | 1.35 | 1.83 | 2.15 |
| 1.2 | 3.27 | 0.82 | 1.28 | 1.25 | 12.68 | 1.37 | 1.72 | 2.12 |
| 1.3 | 2.44 | 0.94 | 1.42 | 1.32 | 14.04 | 1.65 | 2.46 | 2.24 |
| 1.4 | 3.24 | 0.99 | 1.43 | 1.49 | 19.24 | 1.36 | 1.88 | 1.95 |
| 1.5 | 3.17 | 1 | 1.35 | 1.4 | 13.81 | 1.23 | 1.7 | 2 |
| 2.1 | 3.32 | 0.54 | 2.94 | 3.13 | 9.6 | 1.15 | 1.61 | 2.04 |
| 2.2 | 2.94 | 0.86 | 1.78 | 1.88 | 10.87 | 1.16 | 1.73 | 2.05 |
| 2.3 | 2.27 | 0.6 | 2.59 | 2.48 | 8.38 | 1.21 | 1.55 | 2.03 |
| 2.4 | 3.17 | 0.66 | 3.55 | 3.46 | 9.12 | 1.2 | 1.82 | 2.29 |
| 2.5 | 3.57 | 0.59 | 2.33 | 2.23 | 12.11 | 1.46 | 1.61 | 2.18 |
| 3.1 | 2.3 | 0.82 | 1.42 | 1.35 | 11.2 | 1.2 | 1.49 | 2.03 |
| 3.2 | 3.06 | 0.87 | 1.63 | 1.51 | 11.72 | 1.42 | 1.76 | 2.16 |
| 3.3 | 2.81 | 1.02 | 1.69 | 1.76 | 11.54 | 1.07 | 2.31 | 1.97 |
| 3.4 | 3.24 | 1.11 | 2.07 | 1.93 | 12.61 | 1.49 | 2.07 | 2.24 |
| 3.5 | 3.14 | 0.7 | 2.1 | 1.76 | 10.46 | 1.22 | 1.67 | 2.18 |
| 4.1 | 3.14 | 0.76 | 1.6 | 1.59 | 13.38 | 1.52 | 1.97 | 2.25 |
| 4.2 | 3.08 | 1.11 | 1.6 | 1.52 | 14.52 | 1.47 | 2 | 2.2 |
| 4.3 | 2.84 | 0.96 | 1.81 | 1.86 | 15.44 | 1.5 | 2.14 | 2.14 |
| 4.4 | 3.42 | 1.12 | 1.5 | 1.36 | 14 | 1.24 | 2.06 | 2.12 |
| 4.5 | 3.23 | 1.14 | 1.61 | 1.59 | 13.31 | 1.39 | 1.85 | 2.11 |
| 5.1 | 3.06 | 0.52 | 2.19 | 2.05 | 8.52 | 1.03 | 1.56 | 2.05 |
| 5.2 | | | | | | | | |
| 5.3 | | | | | | | | |
| 5.4 | | | | | | | | |
| 5.5 | | | | | | | | |
| 6.1 | 3.26 | 0.78 | 1.78 | 1.82 | 11.11 | 1.13 | 2.28 | 2 |
| 6.2 | 3.26 | 1.2 | 2.14 | 2.06 | 11.9 | 1.44 | 2.48 | 2.21 |
| 6.3 | 2.64 | 0.87 | 2.26 | 2.16 | 10.41 | 1.56 | 2.77 | 2.17 |
| 6.4 | 3.22 | 0.95 | 2.23 | 2.14 | 12.1 | 1.63 | 1.87 | 2.44 |
| 6.5 | 3.28 | 1 | 2.26 | 2.05 | 10.76 | 1.63 | 2.33 | 2.22 |
| 7.1 | 3.27 | 0.94 | 1.76 | 1.66 | 12.3 | 1.51 | 1.46 | 2.02 |
| 7.2 | 2.86 | 0.87 | 1.74 | 1.04 | 12.42 | 1.46 | 1.7 | 2.1 |
| 7.3 | 3.28 | 1.1 | 1.9 | 1.9 | 13.79 | 1.59 | 1.94 | 2.41 |
| 7.4 | 3.26 | 1.2 | 1.67 | 1.68 | 11.59 | 1.56 | 2.01 | 2.26 |
| 7.5 | 3.05 | 1.07 | 1.79 | 1.6 | 13.62 | 1.46 | 1.75 | 2.09 |
| 8.1 | | | | | | | | |
| 8.2 | | | | | | | | |
| 8.3 | 3.24 | 0.55 | 2.07 | 2.03 | 14.08 | 1.29 | 1.42 | 2.29 |
| 8.4 | | | | | | | | |
| 8.5 | | | | | | | | |
| 9.1 | 3.34 | 0.55 | 0.07 | 2.03 | 14.08 | 1.32 | 2.16 | 2.11 |
| 9.2 | 2.98 | 0.77 | 1.99 | 1.87 | 11.55 | 1.43 | 1.82 | 2.01 |
| 9.3 | 3.59 | 0.9 | 2.22 | 2.29 | 12.9 | 1.27 | 1.62 | 2.16 |
| 9.4 | 3.12 | 0.82 | 1.65 | 1.61 | 11.41 | 1.37 | 1.7 | 1.91 |
| 9.5 | 2.89 | 0.89 | 2.02 | 1.97 | 9.17 | 1.04 | 1.49 | 1.97 |
| 10.1 | 3.72 | 0.91 | 1.52 | 1.59 | 15.15 | 1.4 | 2.17 | 2.15 |
| 10.2 | 2.67 | 1.13 | 1.83 | 1.91 | 15.98 | 1.35 | 1.79 | 2.12 |
| 10.3 | 3.21 | 0.78 | 1.61 | 1.59 | 13.5 | 1.18 | 2.02 | 2.14 |
| 10.4 | 3.1 | 0.88 | 1.59 | 1.62 | 12.57 | 1.21 | 1.79 | 2.03 |
| 10.5 | 3.15 | 1.2 | 1.64 | 1.7 | 15.14 | 1.55 | 2.37 | 2.23 |
| 11.1 | | | | | | | | |
| 11.2 | 2.78 | 0.6 | 2.49 | 2.41 | 13.79 | 1.1 | 1.27 | 2.08 |
| 11.3 | | | | | | | | |
| 11.4 | | | | | | | | |
| 11.5 | | | | | | | | |
| 12.1 | 3.01 | 0.61 | 1.87 | 1.9 | 10.71 | 0.99 | 1.39 | 1.98 |
| 12.2 | 2.39 | 0.67 | 2.47 | 2.32 | 8.08 | 0.86 | 1.2 | 1.94 |
| 12.3 | | | | | | | | |
| 12.4 | 3.38 | 0.86 | 2.74 | 3.6 | 13.29 | 1.21 | 1.41 | 2.15 |
| 12.5 | | | | | | | | |
| 13.1 | 3.25 | 0.97 | 2.12 | 2.09 | 14.83 | 1.44 | 1.92 | 2.02 |
| 13.2 | 2.99 | 0.71 | 2.06 | 2.08 | 13.27 | 1.22 | 1.85 | 2.13 |
| 13.3 | 3.25 | 1.1 | 1.81 | 1.73 | 14.15 | 1.47 | 1.79 | 2.14 |
| 13.4 | 3.53 | 1.1 | 2.22 | 2.02 | 13.28 | 1.3 | 1.32 | 2.13 |
| 13.5 | 3.02 | 1 | 1.65 | 1.72 | 12.92 | 1.33 | 1.45 | 1.97 |
| 14.1 | 2.98 | 0.64 | 1.66 | 1.68 | 10.64 | 1.15 | 1.4 | 1.9 |
| 14.2 | 3.12 | 0.85 | 2.33 | 2.29 | 14.59 | 1.31 | 1.77 | 2.12 |
| 14.3 | 2.68 | 0.67 | 2.14 | 2.19 | 9.3 | 0.29 | 1.47 | 1.79 |
| 14.4 | 3.2 | 0.87 | 1.6 | 1.68 | 12.83 | 1.27 | 1.4 | 2.17 |
| 14.5 | 3.14 | 1.1 | 2.75 | 2.73 | 14.35 | 1.5 | 1.56 | 2.16 |
| 15.1 | 3.45 | 0.85 | 2.1 | 2.08 | 11.3 | 1.44 | 2.96 | 2.12 |

Lesions identified in the animals are summarized in Table 14. All lesion catagories (listed below) were scored on a +1-+5 system: 1=minimal, 2=mild, 3=moderate, 4=marked, and 5=severe. Lesions were identified as shown below:

Glomerulopathy—abnormal glomeruli;

Cellular glomerulopathy—(Cell Glom) increased cells in glomeruli—either due to inflammatory infiltrates, or reactive mesangial cells;

Membranous glomerulopathy—increased deposition of mesangial matrix or basement membrane thickening;

Tubular dilation—could be due to two things—less severe dilation was “normal” tubules that had slightly more open lumina—occasionally noted in animals with slight dehydration. More severe dilates (+3 and >) were referring to the appearance of “dilatation” secondary to damaged tubules that were lined by very flattened or attenuated epithelium. This was indicative of severely injured tubules, and although these tubules would ultimately regenerate, depending on the % tubules affected simultaneously, renal failure and death could still result;

Cellular alteration—very subtle degenerative changes in tubular epithelial cells such as mild swelling, pallor of cytoplasm, or slight disorganization of the lining cell;

Tubular regeneration: Evidence of regeneration of tubular epithelial cells characterized by degrees of the following: Karyomegaly/cytomegaly (increased nuclear and cytoplasm size), cytoplasmic basophilia (increased blue staining color indicative of increased RNA), piling up of cells and loss of polarity;

Proteinuria/Protein casts: presence of eosinophilic fluid in tubules—representative of tubular or glomerular damage;

Granular/Cellular casts: Degenerate or necrotic tubular epithelial cells sloughed into tubular lumens—indicative of tubular necrosis;

Protein droplets (accumulation)—these are eosinophilic cytoplasmic droplets in the tubular epithelium. The extreme accumulation, leading to cytomegaly, appears to precede epithelial necrosis and cell sloughage. This may be similar to α2u-globulin nephropathy (α2u-N) seen in several toxicity studies in male rats;

NS=Non-suppurative (no neutrophils);

Within normal limits=WNL.

TABLE 14
Summary of lesions

| Slide/ | | | | Blood | | |
|---|---|---|---|---|---|---|
| group | Glomeruli | Tubules | Interstitium | vessels | Other | Comments |
| 1-1 | WNL | WNL | WNL | WNL | | |
| 1-2 | Focal +2 | Focal +3 dilation | Focal +3 non- | Focal +2 | | Incidental |
| | sclerosis | Focal +1 | suppurative | | | focal infarct |
| | | regeneration | inflammation | | | |
| | | | Focal +2 | | | |
| | | | fibrosis | | | |
| 1-3 | WNL | WNL | WNL | WNL | | |
| 1-4 | WNL | Focal +1 dilation | Focal +1 non- | | | Incidental |
| | | Focal +1 | suppurative | | | focal infarct |
| | | regeneration | inflammation | | | |
| | | | Focal +1 | | | |
| | | | fibrosis | | | |
| 1-5 | WNL | +1 dilation | +1 mixed | WNL | | |
| | | +1 regeneration | inflammation | | | |
| 2-1 | WNL | +4 dilation | +3 non- | WNL | +2 non- | |
| | | +3 cellular casts | suppurative | | suppurative | |
| | | +4 regeneration | inflammation | | pyelitis | |
| | | +5 proteinuria | +2 fibrosis | | | |
| 2-2 | WNL | +2 dilation | +2 non- | WNL | | |
| | | +2 cellular casts | suppurative | | | |
| | | +4 regeneration | inflammation | | | |
| | | +2 proteinuria | | | | |
| 2-3 | +2 dilation | +4 dilation | +3 non- | WNL | | |
| | Bowman's | +4 cellular casts | suppurative | | | |
| | space | +4 regeneration | inflammation | | | |
| | +1 hyper- | +5 proteinuria | | | | |
| | cellularity | | | | | |
| 2-4 | WNL | +4 dilation | +3 non- | WNL | | Looks |
| | | +4 cellular casts | suppurative | | | autolysed |
| | | +4 regeneration | inflammation | | | |
| | | +3 proteinuria | | | | |
| | | +3 mineralization | | | | |
| 2-5 | WNL | +1 dilation | +3 non- | WNL | | |
| | | +2 cellular casts | suppurative | | | |
| | | +4 regeneration | inflammation | | | |
| | | +2 proteinuria | | | | |
| 3-1 | WNL | +2 dilation | +2 non- | WNL | | |
| | | +2 cellular casts | suppurative | | | |
| | | +3 regeneration | inflammation | | | |
| | | +2 proteinuria | | | | |
| 3-2 | WNL | +3 dilation | +2 non- | WNL | | |
| | | +1 cellular casts | suppurative | | | |
| | | +2 regeneration | inflammation | | | |
| | | +1 proteinuria | | | | |
| 3-3 | WNL | +3 dilation | +3 non- | WNL | | |
| | | +2 cellular casts | suppurative | | | |
| | | +3 regeneration | inflammation | | | |
| | | +2 proteinuria | | | | |
| 3-4 | WNL | +3 dilation | +3 non- | WNL | | |
| | | +2 cellular casts | suppurative | | | |
| | | +2 regeneration | inflammation | | | |
| | | +2 proteinuria | | | | |
| 3-5 | WNL | +3 dilation | +3 non- | WNL | | |
| | | +2 cellular casts | suppurative | | | |
| | | +2 regeneration | inflammation | | | |
| | | +2 proteinuria | | | | |
| 4-1 | WNL | +1 dilation | +1 non- | WNL | | |
| | | +2 regeneration | suppurative | | | |
| | | | inflammation | | | |
| 4-2 | WNL | +1 dilation | +2 NS | WNL | | Inflammation |
| | | +1 regeneration | inflammation | | | is perivascular |
| 4-3 | WNL | +1 dilation | +2 NS | WNL | | Inflammation |
| | | +2 regeneration | inflammation | | | is perivascular |
| 4-4 | WNL | +1 regeneration | +1 NS | WNL | | |
| | | | inflammation | | | |
| 4-5 | WNL | +1 regeneration | +1 NS | WNL | | |
| | | | inflammation | | | |
| 5-1 | WNL | +3 dilation | +3 non- | WNL | | |
| | | +2 cellular casts | suppurative | | | |
| | | +4 regeneration | inflammation | | | |
| | | +4 proteinuria | | | | |
| 6-1 | WNL | +3 dilation | +3 non- | WNL | | |
| | | +2 cellular casts | suppurative | | | |
| | | +3 regeneration | inflammation | | | |
| | | +3 proteinuria | | | | |
| 6-2 | WNL | +2 dilation | +4 non- | WNL | | |
| | | +3 cellular casts | suppurative | | | |
| | | +3 regeneration | inflammation | | | |
| | | +3 proteinuria | | | | |
| 6-3 | WNL | +2 dilation | +4 non- | WNL | | |
| | | +3 cellular casts | suppurative | | | |
| | | +3 regeneration | inflammation | | | |
| | | +3 proteinuria | | | | |
| 6-4 | WNL | +1 dilation | +3 non- | WNL | | |
| | | +3 cellular casts | suppurative | | | |
| | | +2 regeneration | inflammation | | | |
| | | +2 proteinuria | | | | |
| 6-5 | +1 | +3 dilation | +3 non- | WNL | | |
| | hyper- | +3 cellular casts | suppurative | | | |
| | cellularity | +4 regeneration | inflammation | | | |
| | | +3 proteinuria | | | | |
| 7-1 | WNL | +1 regeneration | +2 NS | WNL | | Inflammation |
| | | | inflammation | | | is perivascular |
| 7-2 | WNL | MF +3 cellular | +2 NS | WNL | | Not a diffuse |
| | | casts | inflammation | | | lesion |
| | | MF +3 | | | | |
| | | regeneration | | | | |
| | | MF +3 | | | | |
| | | proteinuria | | | | |
| 7-3 | WNL | WNL | WNL | WNL | +4 dilate | |
| | | | | | pelvis or | |
| | | | | | artifact of | |
| | | | | | sectioning | |
| 7-4 | WNL | Focal +2 | +2 NS | WNL | | Autolysed |
| | | regeneration | inflammation | | | |
| 7-5 | WNL | Focal +1 | +2 NS | WNL | | |
| | | regeneration | inflammation | | | |
| | | | +1 focal | | | |
| | | | fibrosis | | | |
| 8-3 | WNL | +3 dilation | +4 non- | WNL | | |
| | | +3 cellular casts | suppurative | | | |
| | | +4 regeneration | inflammation | | | |
| | | +3 proteinuria | | | | |
| 9-1 | WNL | +3 dilation | +4 non- | WNL | | |
| | | +3 cellular casts | suppurative | | | |
| | | +3 regeneration | inflammation | | | |
| | | +2 proteinuria | | | | |
| 9-2 | WNL | +3 dilation | +3 non- | WNL | | |
| | | +3 cellular casts | suppurative | | | |
| | | +3 regeneration | inflammation | | | |
| | | +2 proteinuria | | | | |
| 9-3 | WNL | +2 dilation | +4 non- | WNL | | |
| | | +3 cellular casts | suppurative | | | |
| | | +3 regeneration | inflammation | | | |
| | | +2 proteinuria | | | | |
| 9-4 | WNL | +1 dilation | +2 non- | WNL | | |
| | | +2 cellular casts | suppurative | | | |
| | | +3 regeneration | inflammation | | | |
| | | +2 proteinuria | | | | |
| 9-5 | WNL | +3 dilation | +3 non- | WNL | | |
| | | +3 cellular casts | suppurative | | | |
| | | +4 regeneration | inflammation | | | |
| | | +3 proteinuria | | | | |
| 10-1 | WNL | +1 regeneration | +2 NS | WNL | | |
| | | | inflammation | | | |
| 10-2 | WNL | WNL | +2 NS | WNL | | |
| | | | inflammation | | | |
| 10-3 | WNL | +1 regeneration | +2 NS | WNL | | |
| | | | inflammation | | | |
| 10-4 | WNL | +1 regeneration | +2 NS | WNL | | |
| | | | inflammation | | | |
| 10-5 | WNL | +1 regeneration | +2 NS | WNL | | |
| | | | inflammation | | | |
| 11-2 | WNL | +5 dilation | +3 NS | WNL | | |
| | | +4 regeneration | inflammation | | | |
| | | +3 cellular casts | | | | |
| | | +2 proteinuria | | | | |
| 12-1 | WNL | +4 dilation | +3 NS | WNL | | |
| | | +4 regeneration | inflammation | | | |
| | | +2 cellular casts | | | | |
| | | +2 proteinuria | | | | |
| 12-2 | WNL | +5 dilation | +2 NS | WNL | | |
| | | +4 regeneration | inflammation | | | |
| | | +2 cellular casts | | | | |
| | | +3 proleinuria | | | | |
| 12-4 | WNL | +4 dilation | +2 NS | WNL | | |
| | | +4 regeneration | inflammation | | | |
| | | +2 cellular casts | | | | |
| | | +2 proteinuria | | | | |
| 13-1 | WNL | +3 dilation | +3 NS | | | |
| | | +3 regeneration | inflammation | | | |
| | | +2 cellular casts | | | | |
| | | +2 proteinuria | | | | |
| 13-2 | WNL | +4 dilation | +3 NS | WNL | | |
| | | +4 regeneration | inflammation | | | |
| | | +2 cellular casts | | | | |
| | | +2 proteinuria | | | | |
| 13-3 | WNL | +3 dilation | +2 NS | WNL | | |
| | | +3 regeneration | inflammation | | | |
| | | +2 cellular casts | | | | |
| | | +2 proteinuria | | | | |
| 13-4 | WNL | +3 dilation | +4 NS | WNL | | |
| | | +4 regeneration | inflammation | | | |
| | | +2 cellular casts | | | | |
| | | +3 proteinuria | | | | |
| 13-5 | WNL | +1 dilation | +2 NS | WNL | | |
| | | +3 regeneration | inflammation | | | |
| | | +1 cellular casts | | | | |
| | | +1 proteinuria | | | | |
| 14-1 | WNL | +2 regeneration | +2 NS | WNL | | |
| | | +1 cellular casts | inflammation | | | |
| | | +1 proteinuria | | | | |
| 14-2 | WNL | +3 dilation | +2 NS | WNL | | |
| | | +3 regeneration | inflammation | | | |
| | | +1 cellular casts | | | | |
| | | +1 proteinuria | | | | |
| 14-3 | WNL | +2 dilation | +4 NS | WNL | | |
| | | +4 regeneration | inflammation | | | |
| | | +3 cellular casts | | | | |
| | | +2 proteinuria | | | | |
| 14-4 | WNL | +2 dilation | +2 NS | WNL | | |
| | | +3 regeneration | inflammation | | | |
| | | +2 cellular casts | | | | |
| | | +2 proteinuria | | | | |
| 14-5 | WNL | +1 dilation | +2 NS | WNL | | |
| | | +3 regeneration | inflammation | | | |
| | | +2 cellular casts | | | | |
| | | +2 proteinuria | | | | |
| 15-1 | WNL | +3 dilation | +3 NS | WNL | | |
| | | +3 regeneration | inflammation | | | |
| | | +1 cellular casts | | | | |
| | | +1 proteinuria | | | | |
| 15-2 | WNL | +1 dilation | +1 NS | WNL | | |
| | | +2 regeneration | inflammation | | | |
| | | +1 cellular casts | | | | |
| | | +1 proteinuria | | | | |
| 15-3 | WNL | +2 dilation | +2 NS | WNL | | |
| | | +2 regeneration | inflammation | | | |
| | | +1 cellular casts | | | | |
| | | +1 proteinuria | | | | |
| 15-4 | WNL | +2 dilation | +2 NS | WNL | | |
| | | +3 regeneration | inflammation | | | |
| | | +1 cellular casts | | | | |
| | | +1 proteinuria | | | | |
| 15-5 | WNL | +1 regeneration | +1 NS | WNL | | |
| | | | inflammation | | | |
| 16-1 | WNL | WNL | WNL | WNL | | |
| 16-2 | WNL | WNL | WNL | WNL | | |
| 16-3 | WNL | +3 regeneration | +2 NS | WNL | | Focal lesion |
| | | | inflammation | | | |
| | | | +2 fibrosis | | | |
| 16-4 | WNL | WNL | WNL | WNL | | |
| 16-5 | WNL | +2 cortical cyst | | | | Focal lesions |
| | | +2 dilation | | | | |
| | | +2 regeneration | | | | |

### Biological Example 4

### Activity of Compound 1 Against Various Clinical Isolates

A series of studies were performed to demonstrate the activity of the compound depicted in Representative Compounds Example 1 (“Compound 1,” “the Compound” or “Cmpd”) against common Gram-negative (GN) and Gram-positive (GP) isolates and S. aureus (SA) with resistances (R) to current front-line antimicrobial agents.

In the first study, a total of 235 isolates were collected from medical centers worldwide and tested for susceptibility to Compound 1 and comparator agents by CLSI broth microdilution methods. A total of 125 Enterobacteriaceae (ENT) were tested, including wildtype (WT) strains and those with ESBL, AmpC, KPC, NMC, SME and MBL enzymes. Non-ENT GN pathogens included WT P. aeruginosa (PSA) and Acinetobacter spp. (ACB), and carbapenem (CARB)-R strains, including those with MBL and OXA enzymes, SA and coagulase-negative staphylococci (CoNS) included oxacillin-susceptible (MSSA/MS-CoNS) and —R (MRSA/MR-CoNS) strains.

Minimum inhibitory concentrations (MIC) were determined by reference Clinical and Laboratory Standards Institute (CLSI) broth microdilution methods per M7-A7 [2006]. Briefly, serial two-fold dilutions of Compound 1 and comparator AGs (together, “the test compounds”) were prepared at 2× concentration in appropriate diluent. The dilutions were mixed in 96-well assay plates with bacterial inoculum. Bacteria were suspended in sterile saline and added to each assay plate to obtain a final concentration of 5.5×10⁵ CFU/mL. The plates were incubated at 35° C. for 20 hours in ambient air. The MIC was determined to be the lowest concentration of the test compound that resulted in no visible bacterial growth as compared to untreated control. After incubation, assay plates were read using a microtiter plate reader at 600 nm.

The MIC_{50/90} for Compound 1 was ≦0.25/2, 8/32, 8/32, ≦0.25/0.5 and ≦0.25/≦0.25 μg/ml among ENT, PSA, ACB, SA and CONS, respectively (Table 15). Although AG-R was not a selection criterion for this study, overall sensitivity (S) to gentamicin and amikacin was 66.0 and 77.0%, respectively. There were no differences in the Compound potency against WT strains vs. isolates with R mechanisms with the exception of CARB-R PSA, which were also less sensitive to comparator AGs vs. WT strains. SA, including MRSA, and CoNS were readily inhibited by the Compound.

Compound 1 demonstrated remarkably consistent activity against leading GN pathogens, SA and CoNS, including those with increasingly prevalent R mechanisms.

TABLE 15
Activity of Compound 1

Organism/

| phenotype | Cumulative % inhibited at Cmpd. MIC | | | | | | | | |
|---|---|---|---|---|---|---|---|---|---|
| (no. Tested) | ≦0.5 | 1 | 2 | 4 | 8 | 16 | 32 | 64 | >64 |
| Enterobacteriaceae (125) | 56.0 | 80.0 | 92.0 | 98.4 | 98.4 | 98.4 | 98.4 | 98.4 | 100.0 |
| WT (60) | 41.7 | 70.0 | 88.3 | 100.0 | — | — | — | — | — |
| ESBL (20) | 65.0 | 90.0 | 95.0 | 95.0 | 95.0 | 95.0 | 95.0 | 95.0 | 100.0 |
| KPC, NMC, SME (15) | 60.0 | 100.0 | — | — | — | — | — | — | — |
| AmpC (20) | 75.0 | 85.0 | 95.0 | 95.0 | 95.0 | 95.0 | 95.0 | 95.0 | 100.0 |
| MBL (10) | 80.0 | 80.0 | 90.0 | 100.0 | — | — | — | — | — |
| PSA (30) | 0.0 | 3.3 | 3.3 | 30.0 | 50.0 | 76.7 | 96.7 | 96.7 | 100.0 |
| WT (10) | 0.0 | 10.0 | 10.0 | 70.0 | 100.0 | — | — | — | — |
| CARB-R (20) | 0.0 | 0.0 | 0.0 | 10.0 | 25.0 | 65.0 | 95.0 | 95.0 | 100.0 |
| ACB (30) | 3.3 | 10.0 | 23.3 | 26.7 | 66.7 | 70.0 | 90.0 | 100.0 | — |
| WT (10) | 0.0 | 0.0 | 10.0 | 20.0 | 60.0 | 60.0 | 80.0 | 100.0 | — |
| CARB-R (20) | 5.0 | 15.0 | 30.0 | 30.0 | 70.0 | 75.0 | 95.0 | 100.0 | — |
| SA (30) | 66.7 | 96.7 | 100.0 | — | — | — | — | — | — |
| MSSA (10) | 80.0 | 90.0 | 100.0 | — | — | — | — | — | — |
| MRSA (20) | 60.0 | 100.0 | — | — | — | — | — | — | — |
| CoNS (20) | 100.0 | — | — | — | — | — | — | — | — |

In another study, the activity of Compound 1 against a collection of contemporary Gram-negative bacteria was determined. Unique patient isolates of P. aeruginosa (PA), A. baumannii (AB), K. pneumoniae (KP), E. coli (EC), and Enterobacter spp. (EB) from prior surveys at 16 Brooklyn, N.Y. hospitals were selected based on resistance patterns. The majority of isolates were fingerprinted by automated ribotyping. MICs were performed by broth microdilution. PCR was used to identify aminoglycoside-modifying enzymes (AMEs) in AB and KP and the KPC gene in Enterobacteriaceae (EN). The expression of the efflux genes mexA, C, E and X in PA, and adeB in AB was assessed by real-time RT-PCR.

A total of 204 isolates were tested. Ribotyping revealed that 55% of isolates were unique strains. 44% of the PA and AB isolates were carbapenem-resistant, and 18% of the EN were KPC+. Susceptibility testing (Table 16) revealed that Compound 1 had comparable activity to amikacin against PA and AB, and excellent activity against KP, EC, and EB, including amikacin-resistant and KPC+ strains. Among AB, isolates with the AME aacA4 were more likely to have Compound 1 MICs>4 (93% vs. 50%, P=0.01); however, some isolates without AMEs achieved MICs>16. Among AB, isolates with increased expression of adeB were more likely to have Compound 1 MICs >8 (69% vs. 13%, P<0.001). No relation was found between the presence of AMEs and Compound 1 activity in KP isolates. Among PA, no relation was found between Compound activity and efflux gene expression. Compound 1 showed efficacy against EN, including MDR-KPC+ strains.

TABLE 16
Compound 1 Activity

| | | MIC50 | MIC90 | Susceptible |
|---|---|---|---|---|
| PA (n = 33) | Cmpd. | 8 | 16 | |
| | Amikacin | 4 | 16 | 94% |
| | Gentamicin | 4 | 64 | 67% |
| | Imipenem | 4 | >8 | 55% |
| AB (n = 38) | Cmpd. | 8 | >16 | |
| | Amikacin | 8 | 64 | 82% |
| | Gentamicin | 16 | >64 | 24% |
| | Imipenem | 4 | >8 | 55% |
| KP (n = 71) | Cmpd. | 0.5 | 1 | |
| | Amikacin | 16 | 64 | 58% |
| | Gentamicin | 1 | >64 | 59% |
| | Imipenem | 0.25 | >8 | 79% |
| EC (n = 32) | Cmpd. | 1 | 2 | |
| | Amikacin | 4 | 16 | 91% |
| | Gentamicin | 1 | 64 | 72% |
| | Imipenem | 0.125 | 4 | 91% |
| EB (n = 30) | Cmpd. | 1 | 4 | |
| | Amikacin | 4 | 16 | 93% |
| | Gentamicin | 1 | >64 | 70% |
| | Imipenem | 0.5 | 2 | 93% |
| Amikacin-Resistant-EN | Cmpd. | 0.5 | 2 | |
| (n = 35) | | | | |
| KPC +− EN | Cmpd. | 0.5 | 4 | |
| (n = 24) | | | | |

In a further study, the activity of Compound 1 was assessed against a large collection of AG-resistant and -susceptible clinical isolates. This collection included all of the clinically important AG resistance mechanisms (AGRM). Compound 1 was tested for anti-bacterial activity using the CLSI microbroth dilution method against 461 total clinical isolate strains obtained from diverse geographic regions between 2004 and 2006. These strains consisted of a variety of Gram-negative (Enterobacteriaceae, P. aeruginosa, Acinetobacter) and Gram-positive (S. aureus) organisms with and without AGRM. The AG-modifying enzyme (AME) assignments for six of the most common sequences were confirmed by colony PCR.

Compound 1 showed broad-spectrum antibacterial activity. The potency of Compound 1 was unaffected by most types of AMEs alone or in combinations. The only AME to which Compound 1 was susceptible was AAC(2′)-I, which is a chromosomal enzyme in P. stuartii that may have become less commonly expressed in recent years. Compound 1 was active against the majority of AGRM present in Enterobacteriaceae and, thus, was equally active against populations that are susceptible and resistant to existing AGs.

Further studies analyzed a greater number of unique patient isolates obtained from Brooklyn, N.Y. hospitals (including those species described above). These species included Acinetobacter, E. coli, Enterobacter, Klebsiella, and Pseudomonas. MICs of Compound 1 and other aminoglycosides, including gentamicin (GEN), tobramycin (TOB), amikacin (AMK), meropenem (MEM), ceftazidime (CAZ), cefepime (FEP), ciprofloxacin (CIP), and polymyxcin B (PMX), were determined as described above.

Compound 1 showed broad coverage against these various clinical isolates, as summarized in Table 17. Notably, Compound 1 was more active than amikacin AMK), gentamicin (GEN), or ciprofloxacin (CIP) against E. coli.

TABLE 17
MIC₅₀ and MIC₅₀ of Compound 1 (Cmpd.) and other Aminoglycosides
Against E. coli, Klebsiella, and Enterobacter

| | E. coli | | Klebsiella | | Enterobacter | |
|---|---|---|---|---|---|---|
| | (n = 3071) | | (n = 1151) | | (n = 206) | |
| Compound | MIC₅₀ | MIC₉₀ | MIC₅₀ | MIC₉₀ | MIC₅₀ | MIC₉₀ |
| Cmpd. | 0.5 | 1 | 0.5 | 1 | 0.5 | 1 |
| GEN | 0.5 | 32 | 0.5 | 64 | 0.5 | 16 |
| TOB | 0.5 | 8 | 1 | >64 | 1 | 16 |
| AMK | 2 | 4 | 2 | 32 | 1 | 4 |
| MEM | ≦0.125 | ≦0.125 | ≦0.125 | 8 | ≦0.125 | 0.5 |
| CRO | ≦0.25 | 0.5 | 1 | >32 | ≦0.25 | >32 |
| CAZ | ≦0.25 | 2 | 2 | >32 | 0.5 | >32 |
| FEP | ≦0.25 | ≦0.25 | 0.5 | 32 | ≦0.25 | 8 |
| CIP | ≦0.125 | >4 | 2 | >4 | ≦0.125 | >4 |
| PMX | 1 | 1 | 1 | 2 | 1 | >16 |

Compound 1 also showed strong activity against Acinetobacter and Pseudomonas, as shown in Table 18, and against S. Aureus, as shown in Table 18. Notably, Compound 1 was more active than amikacin against Acinetobacter and euqivalent to amikacin against Pseudomonas.

TABLE 18
MIC₅₀ and MIC₉₀ of Compound 1 (Cmpd.) and other Aminoglycosides
Against Acinetobacter and P. aeruginosa

| | Acinetobacter (n = 407) | | P. aeruginosa (n = 679) | |
|---|---|---|---|---|
| Compound | MIC₅₀ | MIC₉₀ | MIC₅₀ | MIC₉₀ |
| Cmpd. | 8 | 16 | 8 | 32 |
| GEN | 64 | >64 | 2 | >64 |
| TOB | 32 | >64 | 1 | 64 |
| AMK | 32 | >64 | 8 | 16 |
| MEM | >16 | >16 | 2 | 16 |
| CAZ | >32 | >32 | 4 | >32 |
| FEP | >32 | >32 | 8 | 32 |
| CIP | >4 | >4 | 0.5 | >4 |
| PMX | 1 | 2 | 1 | 2 |

### Biological Example 5

### Human Clinical Trials for Compound 1

The Compound depicted in Representative Compounds Example 1 (“Compound 1,” “the Compound” or “Cmpd”) is active against Enterobacteriaceae, including those that express extended spectrum β-lactamases (ESBLs), metallo-β-lactamases, DNA gyrase mutations, and Klebsiella pneumoniae carbapenemases (KPCs). It also has activity against Staphylococcus aureus, including strains resistant to methicillin and vancomycin, and activity against coagulase-negative staphylococci. Importantly, it contains structural modifications that allow it to maintain activity in the presence of nearly all of the aminoglycoside-modifying enzymes (AMEs) that cause aminoglycoside resistance, for which there are approximately 100 known genes. Compound 1 achieves this activity while exhibiting rapid bactericidal killing in vitro and in vivo. Because of these characteristics, Compound 1 is expected to fill an unmet medical need for indications where resistant Gram-negative (e.g., Enterobacteriaceae) and selected Gram-positive pathogens are rendering the older aminoglycosides as well as antibiotics from other drug classes obsolete. In particular, Compound 1 is expected to be efficacious in a number of clinical indications, including complicated urinary tract infections (cUTI), uncomplicated urinary tract infections (uUTI), complicated intra-abdominal infections (cIAI), hospital-acquired pneumonia, and blood stream infections.

Clinical trials are currently being conducted in support of a New Drug Application (NDA) for Compound 1 to treat the two initial indications, cUTI and uUTI. The clinical trials for cUTI include a sufficient number of clinically and microbiologically evaluable patients to investigate the causative microorganisms E. coli, K. pneumoniae, Klebsiella oxytoca, Enterobacter species, Citrobacter species, Serratia marcescens, and Proteus mirabilis, including cases associated with concurrent bacteremia with these microorganisms, as well as P. aeruginosa and Acinetobacter baumannii, both of which are associated with nosocomial cUTI.

The clinical studies by phase are described below and summarized in Table 17. The studies include phase 1 trials to establish the safety and pharmacokinetic (PK) assessments of Compound 1 administered IV in healthy volunteers, in patients with renal insufficiency, and in elderly and pediatric subjects. The initial phase 1 trials in healthy volunteers have been completed, and the results are described below. The program also includes a single, rigorous phase 2 trial of Compound 1 administered IV for the treatment of cUTI to assess the safety, tolerability, efficacy, and pharmacokinetic/pharmacodynamics (PK/PD) of Compound 1 Injection. Finally, the phase 3 program includes two well-controlled, randomized, double-blind trials for the treatment of cUTI comparing Compound 1 administered IV to an established comparator treatment, and one well-controlled, randomized, double-blind, double-dummy trial for the treatment of uUTI comparing a single IM injection of Compound 1 to an established oral comparator treatment.

TABLE 17
Clinical Studies for Compound 1

| Phase | Population | Objectives | Design | Route |
|---|---|---|---|---|
| 1 | Healthy adult | Safety, | Single and multiple | IV |
| | volunteers (18 | tolerability, | escalating doses, PK | |
| | to 55 years) | PK | assessment in adults | |
| 1 | Pediatric subjects | Safety, | PK assessment in | IV |
| | (age groups TBD) | tolerability. | children | |
| | | PK | | |
| 1 | Elderly adults | Safety, | PK assessment in | IV |
| | (>55 years) | tolerability, | elderly | |
| | | PK | | |
| 1 | Adults with renal | Safety, | PK assessment in | IV |
| | insufficiency | tolerability, | renal insufficiency | |
| | | PK | | |
| 1 | Healthy adult | Safety, | PK assessment of | IV |
| | volunteers | tolerability, | IM formulation | |
| | (≧18 years) | relative BA | | |
| | | and PK | | |
| 2 | Adult patients | Safety, | Randomized, double- | IV |
| | with cUTI | tolerability, | blind, with dose and | |
| | | efficacy, | duration assessments | |
| | | PK/PD | | |
| 3 | Adults with | Pivotal | Randomized, double- | IV |
| | cUTI | efficacy | blind, comparator- | |
| | | | controlled | |
| 3 | Adults with | Pivotal | Randomized, double- | IV |
| | cUTI | efficacy | blind, comparator- | |
| | | | controlled | |
| 3 | Adults with | Pivotal | Randomized, double- | IM |
| | uUTI | efficacy | blind, comparator- | |
| | | | controlled | |

cUTI = complicated urinary tract infection; IM = intramuscular; IV = intravenous; PD = pharmacodynamics; PK = pharmacokinetics; TBD = to be determined; uUTI = uncomplicated urinary tract infection.

### Phase I Clinical Trial

The initial, first-in-human (FIH) phase 1 study was a double-blind, randomized, placebo-controlled, parallel-group, single- and multiple-dose-escalation study to assess the safety, tolerability, and pharmacokinetics of Compound 1 administered IV to healthy adult volunteers. In addition, the design of this FIH phase 1 study provided rationale and support for dosing for the proposed phase 2 study in patients with cUTI (see FIG. 11).

This study was designed with four escalating dose arms in both single-dose and multiple-dose phases. Compound 1 was formulated as a sterile solution at a concentration of 50 mg/mL in 10 mL glass vials for intravenous (IV) administration (“Compound 1 Injection”). To support high-dose, short-course therapy, the FIH phase 1 study investigated escalating doses with shorter durations at the higher dosages. At least 32 male and female healthy subjects (8 per Cohort) were randomized to receive a single dose of either Compound 1 Injection or placebo administered IV by 10 minute infusion in a double-blinded manner, as shown below:

Cohort 1a: 1 mg/kg Single Dose (SD) only

Cohort 1b: 4 mg/kg SD and Multiple Doses (MD) for 10 days

Cohort 2: 7 mg/kg SD and MD for 10 days

Cohort 3: 11 mg/kg SD and MD for 5 days

Cohort 4: 15 mg/kg SD and MD for 3 days

The study design provided adequate safety evaluation before commencement of dose escalation by mandating ample review time (at least 7 days) between the start of each dosing arm. The multiple-dose period of the study followed the single-dose period, beginning after a 7-day evaluation period.

The Food and Drug Administration (FDA) has provided general guidance for estimating the maximum recommended starting dose (MRSD) for initial human clinical trials. The guidance states that the MRSD must be derived from the no observable adverse effect level (NOAEL) obtained in Good Laboratory Practice (GLP) nonclinical animal safety studies. The NOAEL for Compound 1 has been determined in GLP 14-day repeat-dose safety studies in both rat and dog. Estimation of the human equivalent dose (HED) at the NOAEL is based on both a surface area conversion factor (SACF) and the data from these studies. Based on this standard methodology, the HED for the predicted NOAEL in each species has been determined and is presented in Table 18.

TABLE 18
Estimated HED for Compound 1 from NOAELs in Rats and Dogs

| GLP Study | | NOAEL | | HED |
|---|---|---|---|---|
| Species | Duration | (mg/kg) | SACF^{a} | (mg/kg) |
| Rat | 14 days | 8 | 6.2 | 1.3 |
| Dog | 14 days | 3 | 1.8 | 1.7 |

GLP = Good Laboratory Practices; HED = human equivalent dose; NOAEL = no adverse effect level; SACF = surface area conversion factor.
^{a}The HED was calculated based on standard surface area (m²) to kilogram (kg) conversion factors, 6.2x for rat and 1.8x for dog compared to human subjects.

The MRSD is generally based on the HED at the NOAEL dose level in the most sensitive species. The rat is consistently regarded as the most sensitive species for determining NOAEL for aminoglycosides. As a class, aminoglycosides show a relatively shallow dose response for nephrotoxicity based on kidney histopathology. Thus, the NOAEL levels are based on this sensitive readout, despite the fact that the subtle histologic changes are mild and reversible at this dose level. The NOAEL for gentamicin, based on the presence of subtle histopathologic changesin kidneys of rats after 14 days of dosing is less than 1 mg/kg, compared to the dose of 30 mg/kg, the lowest dose that causes a detectable change in serum chemistry values (serum creatinine and BUN) after 14 days of dosing.

The standard safety factor used to obtain the MRSD from the HED based upon NOAELs is a 10-fold safety margin. However, in the case of Compound 1 Injection, there is a reasonable rationale for lowering the safety factor, based on the following:
Compound 1 is a member of a well-characterized class, namely the aminoglycoside class;
the toxicity, metabolic, and clearance profile for members of the aminoglycoside class in clinical use is similar across the nonclinical animal models (rats and dogs) and humans;
the NOAEL-defining and dose-limiting toxicity of this class is nephrotoxicity, which is easily monitored, and is reversible upon cessation of treatment;
the duration of therapy for Compound 1 Injection in this human trial of 10, 5, and 3 days, is less than the duration in animal studies used to define the NOAEL (14 days);
the intended clinical route of Compound 1 Injection is the same as other established members of the class currently used in the clinic;
there have been no new toxicities apart from those already known to the class in nonclinical GLP safety studies in rats and dogs; and
there are no cardiac or respiratory safety signals identified with Compound 1 in nonclinical GLP studies in dogs;

The favorable pharmacokinetic profile seen with aminoglycosides, combined with strong scientific evidence of their safety and efficacy, and a more recent, deeper understanding of their pharmacodynamic relationships, including the results described in the previous Examples, all support the rationale for higher doses administered once daily for short durations. As such, optimal dosing of Compound 1 was based on consideration of the following factors:
delivering an effective AUC relative to the MIC of the causative pathogen;
delivering a high Cₘₐₓ for a given AUC to maximize efficacy;
delivering a high Cₘₐₓ for a given AUC to deliver as much of the daily dose above the saturable uptake threshold of renal proximal tubule cell;
delivering a high Cₘₐₓ for a given AUC to prevent development of resistance;
administering once-daily doses in short infusion times (e.g., 10 minutes) is the most efficient means for delivering a high Cₘₐₓ for a given AUC; and
administering therapy in short durations to further minimize the risk of toxicity.

Safety evaluation was based on clinical observations, vital sign measurements, laboratory tests, physical examination, electrocardiogram, cochlear function, vestibular function testing, and reported adverse events. Cochlear function was evaluated by pure tone audiometry with bone conduction and otoacoustic emission (OAE) testing. Vestibular function was evaluated by electronystagmography (ENG) and Dynamic Visual Acuity (DVA) test. Laboratory tests included complete blood count (CBC), serum chemistries, and urinalysis. BUN and creatinine (Cr) were assessed daily on dosing days to allow the Investigator to monitor renal function. Results were used as criteria for dose escalation. The glomerular filtration rate (GFR) was estimated using iothalamate plasma measurements before dosing and after the multiple dose periods. GFR was also assessed by Cr clearance before dosing and after the multiple dose periods.

Blood and urine samples for safety laboratory tests were obtained during the screen period, before and after the single-dose, before and after the multiple-dose (additional samples were obtained during multiple-dose period depending on MD duration). Blood samples for PK analysis were obtained with the single dose and first and last multiple-doses at the following times:

Predose;

5, 10, 15, 20, 30, 45, 60 minutes after the start of the infusion; and

1, 1.5, 2, 2.5 (Cohorts 3 and 4 only), 3, 4, 6, 8, 12, 16, 24, and 48 hours after the start of the infusion.

For subjects in the 10-day multiple-dose cohorts, additional blood samples for PK analysis were obtained predose on days 11, 13, and 15.

Pooled urine samples for PK analysis were obtained with the single dose and first and last multiple-dose. 24-hour pooled urine for Cr clearance was obtained with the single-dose and with the first and last dose of the multiple-doses.

Iothalamate was infused the day prior to the single-dose and prior to the first multiple-dose and the day after the last multiple-dose. Blood samples for iothalamate analysis for GFR calculation were obtained prior to the start of the iothalamate infusion and the following hours after the start of the infusion: 1, 2, 4, and 8.

Complete physical examinations and ECGs were completed at screen and 2 days after the last multiple-dose. Abbreviated physical examinations were completed prior to the single-dose and prior to the first multiple-dose (those receiving IMP for 10 days have an additional examination prior to the 6^{th} multiple-dose infusion). Vital signs were assessed at screen and all visits during treatment period. Additional safety assessments, including pure tone audiometry and ENG caloric, testing was performed or will be performed at 3 and 6 months after the last dose. Concomitant therapies and adverse events are assessed throughout the screen and treatment period.

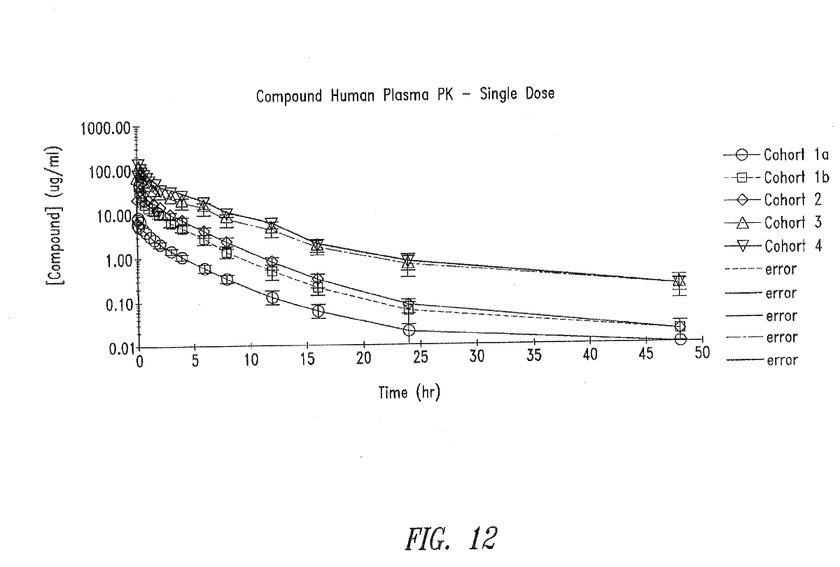

Pharmacokinetic analysis of the phase 1 trial results demonstrated a clear dose proportionality of Compound 1 over the concentrations of Compound 1 tested in the various cohorts. A graph depicting the average concentration of Compound present in patient serum over the 48 hours following a single administration is shown for each of the Cohorts in FIG. 12. As shown in Table 19, Cₘₐₓ increased proportionally with the concentration of Compound 1. Furthermore, the derived serum clearance rate remained constant over the various concentrations of Compound 1. Iothalamate plasma measurements similarly did not change, demonstrating that the glomerular filtration rate

TABLE 19
Human Plasma pK of Compound 1—Single dose—48 hours

| | PK parameter | | | | |
|---|---|---|---|---|---|
| | | | t½ | t½ | CL * |
| | Cmax * | AUC 0-∞ | alpha ** | beta ** | (mL/hr/ |
| | (ug/mL) | (hr*ug/mL) | (hr) | (hr) | kg) |
| Cohort 1a | 7 ± 0.7 | 14 ± 1 | | | 74 ± 6 |
| (1 mg/kg) | | | | | |
| Cohort 1b | 32 ± 5 | 61 ± 8 | | | 67 ± 7 |
| (4 mg/kg) | | | | | |
| Cohort 2 | 46 ± 7 | 88 ± 12 | | | 81 ± 11 |
| (7 mg/kg) | | | | | |
| Cohort 3 | 114 ± 27 | 176 ± 34 | | | 65 ± 12 |
| (11 mg/kg) | | | | | |
| Cohort 4 | 144 ± 45 | 242 ± 37 | 3 | 15 | 63 ± 10 |
| (15 mg/kg) | | | | | |

* Parameters (+Standard Deviation) determined by noncompartmental analysis
** Parameters determined by compartmental (2) analysis

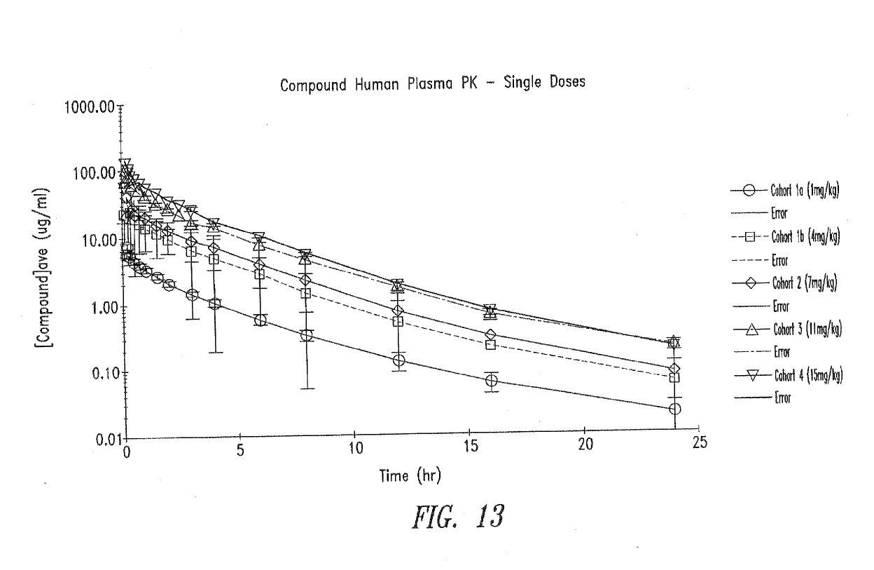

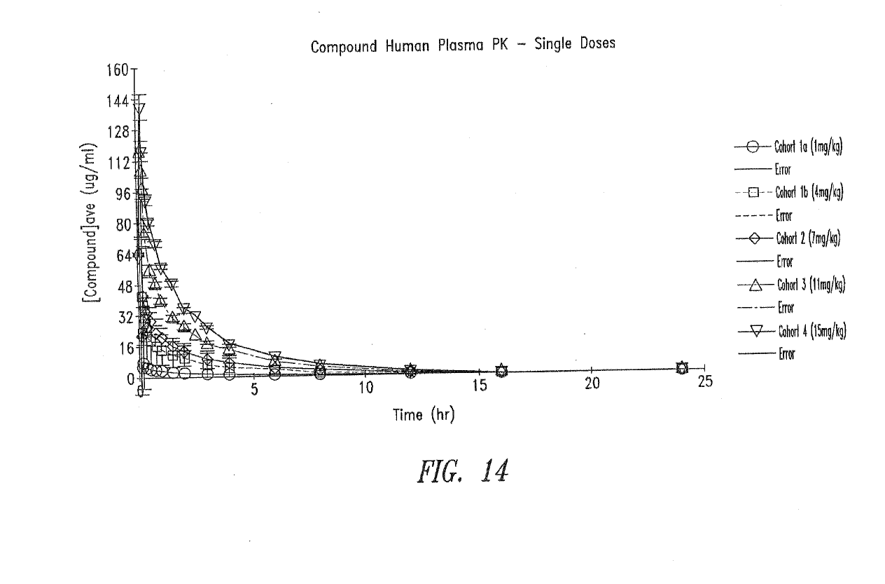

A log graph depicting the average concentration of Compound 1 present in patient serum over the 24 hours following a single administration is shown for each of the same Cohorts examined above in FIG. 13. The same data plotted linearly is shown in FIG. 14. As summarized below in Table 20, dose proportionality was clearly demonstrated over the dosages examined, and serum clearance remained constant.

TABLE 20
Human Plasma pK of Compound 1—Single dose—24 hours

| | PK parameter estimates | | | | |
|---|---|---|---|---|---|
| | | | t_{1/2} | t_{1/2} | CL |
| | Cmax | AUC 0-∞ | alpha | beta | (mL/hr/ |
| | (ug/mL) | (hr*ug/mL) | (hr) | (hr) | kg) |
| Cohort 1 a | 8 ± 0.8 | 15 ± 1 | 0.5 ± 0.1 | 3.5 ± 0.2 | 68 ± 4 |
| (1 mg/kg) | | | | | |
| Cohort 1b | 38 ± 4 | 65 ± 4 | 0.3 ± 0.1 | 3.0 ± 0.1 | 62 ± 4 |
| (4 mg/kg) | | | | | |
| Cohort 2 | 40 ± 3 | 89 ± 4 | 0.7 ± 0.1 | 3.1 ± 0.1 | 78 ± 3 |
| (7 mg/kg) | | | | | |
| Cohort 3 | 106 ± 10 | 186 ± 8 | 0.3 ± 0.1 | 3.1 ± 0.1 | 59 ± 3 |
| (11 mg/kg) | | | | | |
| Cohort 4 | 116 ± 8 | 250 ± 9 | 0.8 ± 0.1 | 3.1 ± 0.2 | 60 ± 2 |
| (15 mg/kg) | | | | | |

** All parameters (±Standard Error) determined from averages by compartmental (2) anlaysis using WinNonLin

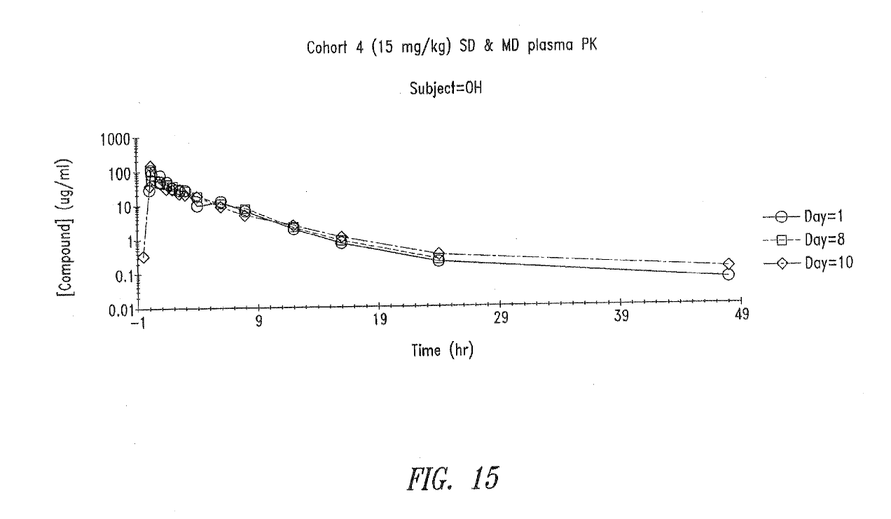

Plasma levels over 48 hours of Compound 1 in a single Cohort 4 patient following the single dose, the first multi-dose, and the third multi-dose administration of Compound 1 are shown in FIG. 15. Since the first multi-dose was adminstered to the patient seven days following administration of the single dose, it was not surprising that the serum concentrations over time were very similar for these two administrations. However, it was notable that the serum concentrations for the third multi-dose were also very similar to the other doses, since the patient had received doses every 24 hours for three days. These data indicated that there was very little, in any, accumulation of Compound 1 over three days. This overlay of serum concentrations over time was not unique to the patient examined; rather, a similar overlay was observed for all dose levels.

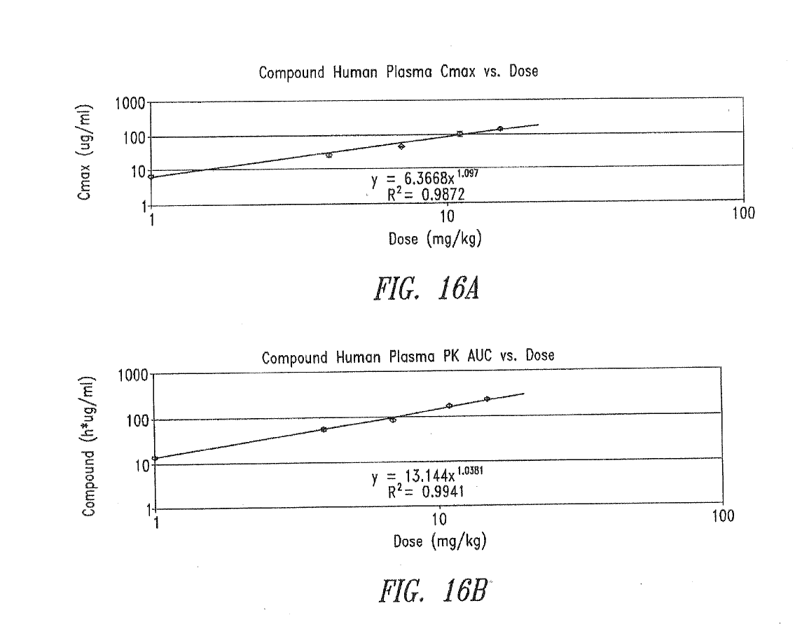

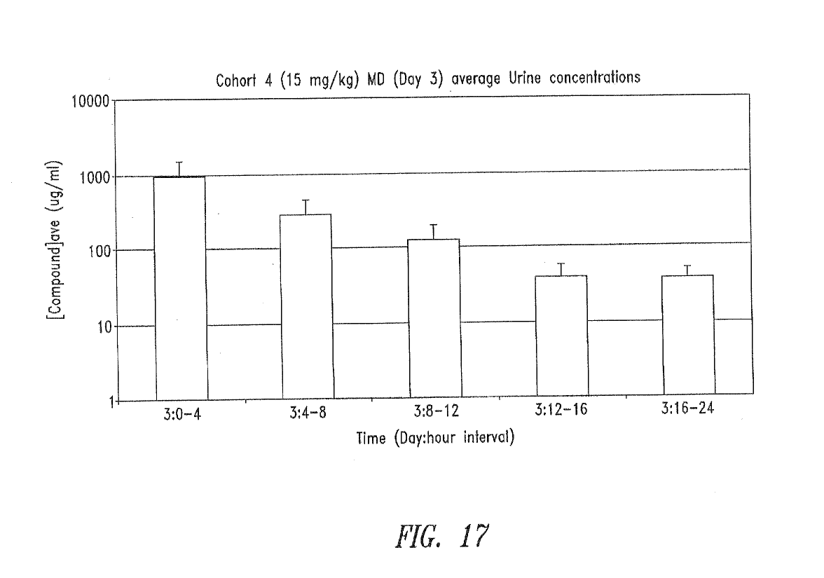

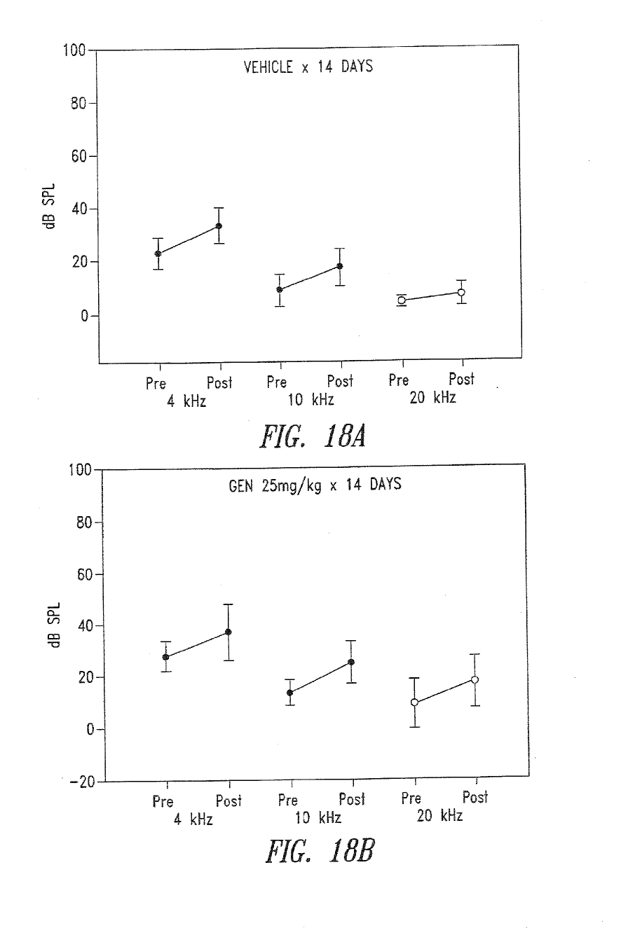

The tight dose proportionality of plasma concentrations of Compound 1 is further demonstrated in FIG. 16. The top panel depicts Cₘₐₓ at the various doses administered to the Cohorts, showing a linear increase in Cₘₐₓ as dose increased. Similarly, as shown in the lower panel, AUC also increased linearly as the dose increased.

The levels of Compound 1 present in the urine of Cohort 4 patients during the 24 hours following administration of the third multi-dose was also examined. As shown in FIG. 17, the average fraction of Compound 1 excreted in an unchanged form over the 24 hours was 75%±17%, indicating that as much as about 90% of administered Compound 1 was excreted within 24 hours. Perhaps of even greater significance, the concentration of Compound 1 achieved in the urine was at least 1 mg/ml immediately following administration, and remained over 0.1 mg/ml for 12 hours. This level is substantially higher than the MIC of a variety of bacteria associated with urinary tract infections, thus indicating that Compound 1 should be efficacious in treating urinary tract infections at the higher tested dosages.

In the phase 1 clinical studies described herein, Compound 1 was administered over a short infusion time period of 10 minutes. Based upon allometric scaling of the mouse, rat and dog nonclinical data, the Cₘₐₓ associated with longer infusion time periods was predicted, as summarized in Table 21. As shown, longer infusion time periods are associated with lower Cₘₐₓ, resulting in a lower Cₘₐₓ to AUC ratio. This data supports the use of short infusion time periods for administration of Compound 1, in order to maximize the ratio of Cₘₐₓ to AUC and enhance efficacy.

TABLE 21
Prediction of key plasma levels and exposures of Compound 1 in humans
following IV infusions at different infusion rates and dose levels

| Dose | Infusion time (hr) | | | | | | | | | | | |
|---|---|---|---|---|---|---|---|---|---|---|---|---|
| level | 0.167 | | | 0.333 | | | 0.5 | | | 1 | | |
| (mg/kg) | Cₘₐₓ | AUC | Cₘₐₓ/AUC | Cₘₐₓ | AUC | Cₘₐₓ/AUC | Cₘₐₓ | AUC | Cₘₐₓ/AUC | Cₘₐₓ | AUC | Cₘₐₓ/AUC |
| 7 | 48 | 89 | 0.54 | 45 | 89 | 0.51 | 43 | 89 | 0.48 | 37 | 89 | 0.42 |
| 11 | 75 | 140 | 0.54 | 71 | 140 | 0.51 | 68 | 140 | 0.49 | 59 | 140 | 0.42 |
| 15 | 102 | 191 | 0.53 | 97 | 191 | 0.51 | 92 | 191 | 0.48 | 80 | 191 | 0.42 |

No ototoxocity signal was observed for Compound 1. The ENG and DVA results did not reveal any treatment related effects of Compound 1 on vestibular function. One subject in the 4 mg/kg group with an abnormal ENG finding at baseline that was recorded as a slight variation from normal, also had a TEAE of vestibular function test abnormal, but this baseline finding did not change with treatment. Changes from baseline in DVA assessment were all within the acceptable variation for the test (Delta=−1) and did not represent abnormalities or treatment related effects.

The pure tone audiometry and bone conduction and OAE results did not reveal any treatment related effects from Compound 1 on cochlear function. There were no abnormal pure tone audiometry and bone conduction results. The few OAE abnormalities observed were associated with other clinical events such as upper respiratory infections or nasal congestion and not considered to be related to Compound 1.

Overall, the results of the FIH phase 1 clinical trials demonstrated that Compound 1 exhibits dose-proportional Cₘₐₓ and AUC, while clearance remained constant. In addition, therapeutic concentrations of Compound 1 were achieved using a high dose, short infusion, short course dosing regimen.

An additional randomized, double-blind, placebo-controlled study was performed to assess the safety and pharmacokinetics of Compound 1 following IV administration to healthy adult volunteers according to a high dose, short course regimen of 15 mg/kg/day of Compound 1 administered once daily for five days. Compound 1 was formulated as a sterile solution at a concentration of 50 mg/mL in 10 mL glass vials for intravenous (IV) administration (“Compound 1 Injection”). Eight healthy subjects were randomized to receive either Compound 1 Injection or placebo administered IV by 10 minute infusion daily for five days. Seven subjects received 5 doses and one received 3 doses (early withdrawal for personal reasons). All adverse events were mild to moderate.

No evidence of ototoxicity or nephrotoxicity was observed through the treatment period. BUN and creatine (Cr) were assessed daily for each of the five days prior to dosing and at day 5 post-dosing to assess renal function following this high dose, short course regimen. As shown in Tables 22 and 23, no substantial changes in either BUN or Cr were observed at day 5 post-dosing when compared to the functions observed the five days prior to dosing, supporting the safety of Compound 1 in a high dose, short infusion, short course dosing regimen.

TABLE 22
BUN measurements for high dose, short
course treatment with Compound 1

| | BUN (mg/dL) | | | | | |
|---|---|---|---|---|---|---|
| Subject | Pre- | Pre- | Pre- | Pre- | Pre- | Post- |
| ID | dose 1 | dose 2 | dose 3 | dose 4 | dose 5 | dose 5 |
| 1001 | 19 | 22 | 18 | 19 | 16 | 20 |
| 1002 | 14 | 17 | 14 | 16 | 16 | 15 |
| 1003 | 9 | 18 | 13 | 13 | 16 | 13 |
| 1004 | 9 | 16 | 18 | 14 | | |
| 1005 | 10 | 19 | 19 | 12 | 13 | 13 |
| 1007 | 13 | 17 | 17 | 14 | 14 | 14 |
| 1008 | 5 | 8 | 9 | 8 | 11 | 9 |
| 1009 | 15 | 20 | 15 | 17 | 15 | 13 |

TABLE 23
Cr measurements for high dose, short
course treatment with Compound 1

| | Serum Creatine (mg/dL) | | | | | |
|---|---|---|---|---|---|---|
| Subject | Pre- | Pre- | Pre- | Pre- | Pre- | Post- |
| ID | dose 1 | dose 2 | dose 3 | dose 4 | dose | dose 5 |
| 1001 | 1.0 | 1.1 | 0.9 | 0.9 | 1.1 | 0.9 |
| 1002 | 0.9 | 0.9 | 0.8 | 0.9 | 1.0 | 0.9 |
| 1003 | 0.7 | 0.7 | 0.6 | 0.6 | 0.7 | 0.6 |
| 1004 | 0.6 | 0.6 | 0.6 | 0.6 | | |
| 1005 | 0.7 | 0.9 | 0.7 | 0.6 | 0.8 | 0.6 |
| 1007 | 0.9 | 1.1 | 1.0 | 1.0 | 1.2 | 1.0 |
| 1008 | 0.6 | 0.6 | 0.6 | 0.6 | 0.7 | 0.7 |
| 1009 | 1.2 | 1.3 | 1.1 | 1.2 | 1.2 | 1.1 |

After 5 days of repeated once-daily IV dosing of Compound 1, the area under the plasma concentration-time curve, plasma clearance and steady state volume of distribution of Compound 1 averaged 239 hr*μg/mL, 1.1 mL/min/kg and 0.24 L/kg, respectively. The half-life was approximately 3 hours. The peak (Cₘₐₓ) and trough (Cₘᵢₙ) was 113 μg/mL and 0.4 μg/mL, respectively. The fluctuation index value averaged >1000%.

In this study, Compound 1 was well-tolerated and exhibited PK parameters supportive of once daily, high dose, short course therapy for aminoglycosides. The lack of appreciable drug accumulation after once-daily dosing for up to 5 days is consistent with the apparent elimination half-life of approximately 3 hours.

Additional phase 1 PK studies investigating Compound 1 Injection in special populations are conducted before commencement of any phase 3 studies. These trials are designed to assess the safety, tolerability, and PK of Compound 1 Injection in subjects with renal insufficiency, elderly subjects, and pediatric subjects.

### Phase 2 Clinical Trials

A phase 2 trial is conducted in patients with cUTI. This phase 2 trial is a doubleblinded study to assess the safety, tolerability, dose-comparison, duration of therapy, efficacy, PK, and PD of Compound 1 Injection. Since the PK and bactericidal properties of aminoglycosides favor high-dose, short-course therapy, the trial evaluates three dose levels and three durations of dosing (10, 5, and 3 days) of Compound 1 Injection in a double-blind design (Table 22). In addition, this study includes sparse sampling of serum for PK drug concentrations. Urine samples are collected in selected patients with in-dwelling catheters for measurement and analysis of Compound 1. A PK/PD analysis is conducted to assess the dose-response relationships of efficacy and safety in patients with cUTI.

TABLE 22
Dose Groups in Phase 2 Study of Patients with cUTI

| Dose Group | Duration | |
|---|---|---|
| (Dose in mg/kg) | (Days) | Design (Days) |
| 7 | 10 | 10 days active |
| 10 | 5 | 5 days active + 5 days placebo |
| 10 | 3 | 3 days active + 7 days placebo |
| 15 | 3 | 3 days active + 7 days placebo |

### Phase 3 Clinical Trials

To meet the criteria established in the FDA Guidance Documents for the indication of cUTI, the phase 3 program includes two well-controlled, randomized, double-blind, trials for the treatment of cUTI comparing Compound 1 Injection to an established comparator treatment. The trial is statistically powered to establish noninferiority to the comparator agent.

To meet the criteria established in the FDA Guidance Documents for the indication of uUTI, the phase 3 program includes one well-controlled, randomized, double-blind trial for the treatment of uUTI comparing a single IM injection of Compound 1 to an established comparator treatment. The trial includes a double-dummy design because most approved comparator agents are administered orally. The trial is statistically powered to establish noninferiority to the comparator agent.

These clinical studies are expected to establish the efficacy of a high dosage, short course dosing regiment of Compound 1 for the treatment of cUTI and uUTI.

### Biological Example 6

### Comparison of the Ototoxicity of Aminoglycosides and Compound 1

Ototoxicity is a potential toxicity associated with the aminoglycoside class of antibiotics. Ototoxicity can be either auditory or vestibular and is generally correlated with longer durations of therapy and total cumulative doses, particularly total cumulative AUC. The ototoxic potential of known aminoglycosides and the Compound depicted in Representative Compounds Example 1 (“Compound 1,” “the Compound” or “Cmpd”) was examined in guinea pigs. The guinea pig is a universally used model for evaluating the potential ototoxicity of various classes of chemicals and for which there is a large historical database. In addition, due to its unique physiological and neuroanatomical characteristics, the guinea pig is the standard species contemporarily employed on studies such as this to evaluate the potential ototoxicity of investigational new drugs.

As described below, guinea pigs dosed for 14 days with up to 80 mg/kg/day of Compound 1 showed no functional hearing loss, and Compound 1 depicted in Example 1 appeared to have less potential ototoxicity as measured by ABR in guinea pigs than gentamicin. These studies further demonstrate that dosing strategies that deliver aminoglycosides as a single daily dose and in short courses (3-5 days) may decrease the risk of ototoxicity, while simultaneously maximizing efficacy.

### Ototoxicity of Aminoglycosides in 28-Day Study in Guinea Pigs

The ototoxicity of aminoglycosides was investigated in 28-day studies in guinea pigs (executed at MPI Research Inc., Mattawan, Mich.). The aminoglycoside comparators gentamicin and amikacin were used in preliminary studies to demonstrate that the guinea pig study design is able to show ototoxicity for this class of compounds. In these studies, gentamicin and amikacin were administered subcutaneously once-daily for 14 days, and evidence of ototoxicity was investigated 14 days later. Six treatment groups of six male experimentally naïve Crl:HA (Albino Hartley) guinea pigs (obtained from Charles River Laboratories, Raleigh, N.C., USA) were administered the positive control articles, gentamicin or amikacin, at respective dose levels of 25, 50, and 100 mg/kg for gentamicin and 75, 150, and 300 mg/kg for amikacin (Groups 2-7, respectively). One additional group of six male animals served as the control and received the vehicle, 0.9% Sodium Chloride for Injection, USP, in the same manner as the treatment groups (Group 1). Animals assigned to the study had body weights within ±20% of the mean body weight. The vehicle and positive control articles were administered daily for 14 days during the study via bolus subcutaneous injection in the scapular region on the back of each animal. The control group received the vehicle in the same manner as the treated groups. Individual doses were based on the most recent body weights. The positive control articles or vehicle were administered to all groups at a dose volume of 1 mL/kg. The dose levels for these studies were selected based upon published accounts of aminoglycoside ototoxicity in the guinea pig using similar dosing regimes (e.g., Brummett, A. R. et al., J. Antimicrob Chemother, 1978 May; 4 Suppl A:73-83; Kitasato, I. et al., Chemotherapy, 1990; 36(2):155-68). Doses were chosen to maximize exposure while avoiding significant nephrotoxicity, which would have confounded the analysis given that aminoglycosides are renally cleared.

Observations for morbidity, mortality, injury, and the availability of food and water were conducted twice daily for all animals. Clinical observations were conducted predose (Day 1), immediately following the completion of each vehicle or positive control article administration on dosing days, and daily thereafter. Body weights were measured and recorded predose (Day 1) and daily thereafter. Physical examinations were conducted pretest and on Day 28.

Bilateral in-vivo electrophysiology (auditory brainstem response) examinations were conducted pretest and prior to the terminal necropsy. Hearing function changes were measured by comparing auditory brainstem response (ABR) values at the end of the study period versus pre-dosing values of each individual animal. The examination was conducted according to Kresge Hearing Research Institute method at three different frequencies (4, 10, and 20 kHz) while animals were anesthetized with a combination of ketamine (15 mg/kg) and xylazine (2.5 mg/kg) and placed in a portable, sound-attenuated, electrically shielded enclosure. Additional ketamine and xylazine were administered as necessary. The animals were fasted the morning of the ABR examinations. Following ABR evaluations, yohimbine (1 mg/kg) was administered via intraperitoneal (IP) injection as a reversal agent. At study termination (Day 29), necropsy examinations were performed, and designated tissues were collected, examined and rated, and preserved for processing and histopathological evaluation, as described below.

Following euthanasia and whole body intravascular perfusion with 4% paraformaldehyde, the temporal bones of the left and right ears were removed. Gross middle ear assessments were performed using a dissecting microscope. During the examination, the middle ear was opened by removing regions of the temporal bone until the ossicles were revealed. Following gross middle ear assessment, the cochleas of the left ear were fixed by intrascalar perfusion with 4% paraformaldehyde. Cochlea tissues of both ears were placed in 4% paraformaldehyde for approximately 1 hour. Tissues were then transferred to 0.5% paraformaldehyde, stored refrigerated, and shipped on gel packs to the Kresge Hearing Research Institute, Ann Arbor, Mich., for further processing and cytocochleogram analysis.

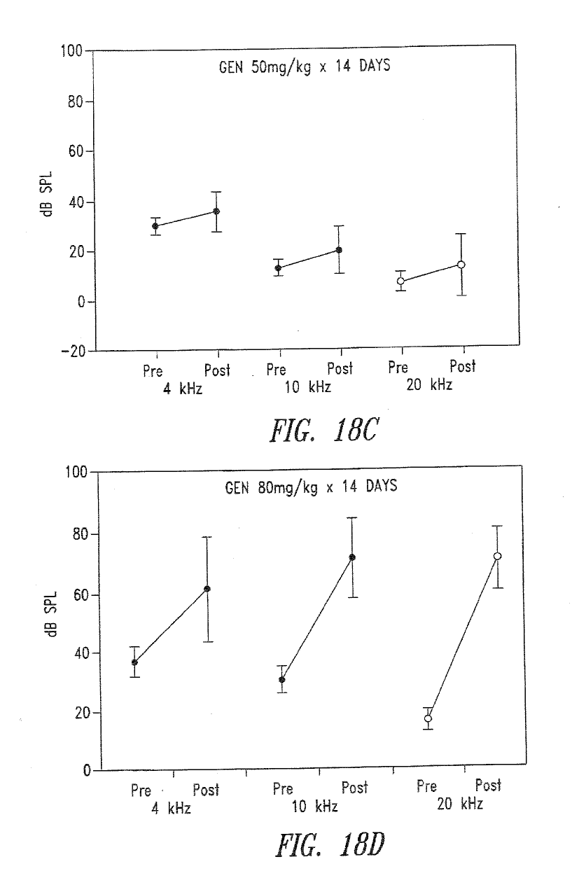

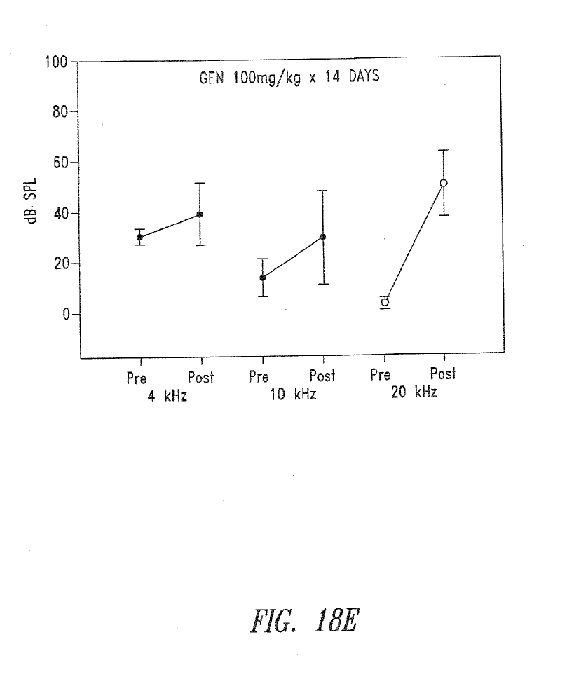

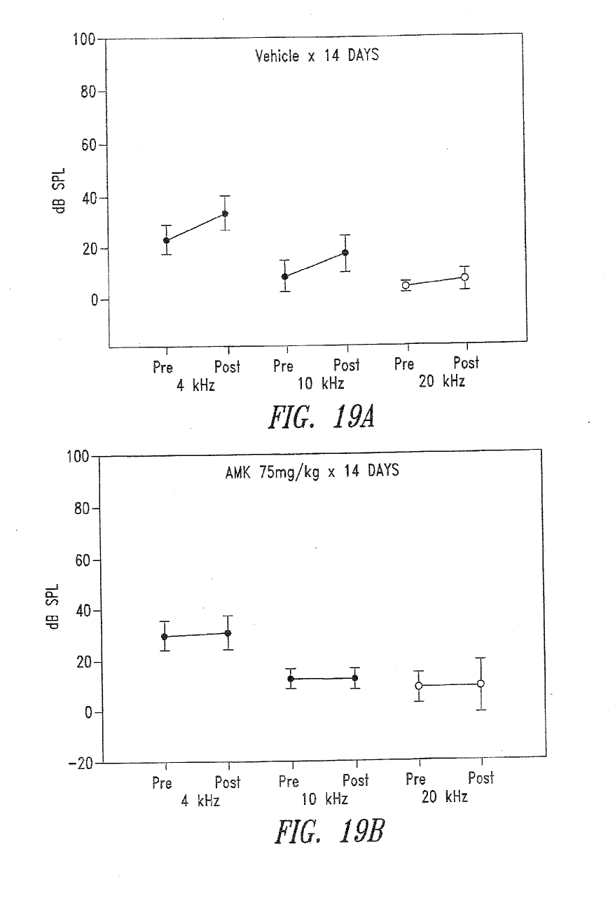

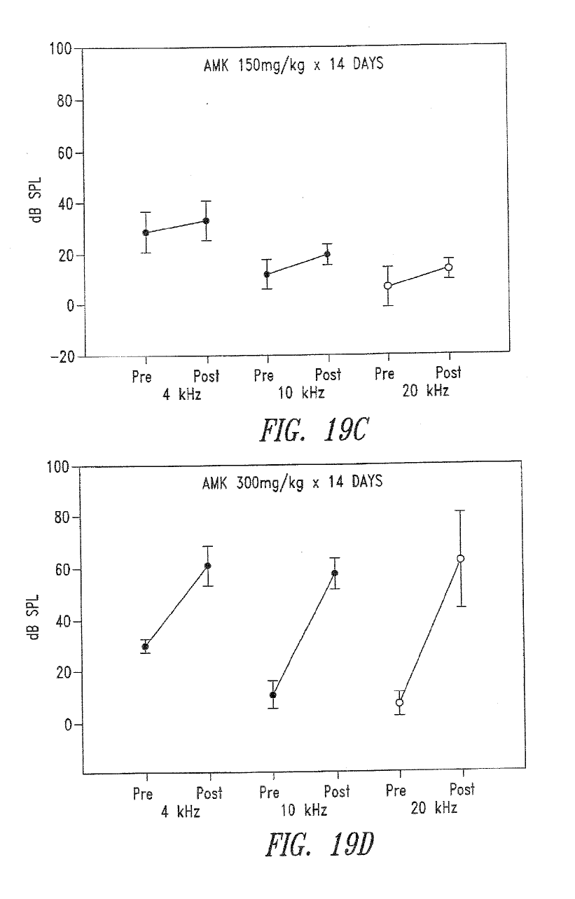

Clinical observations, body weights, physical examinations, and macroscopic observations showed no vehicle or positive control article-related changes in any dose group. Threshold changes observed in the in-vivo electrophysiology examinations were suggestive of sensorineural loss of cochlear origin in the gentamicin 100 mg/kg group with hearing loss at 10 and 20 kHz (FIG. 18), and the 300 mg/kg amikacin group with hearing losses from moderate at 4 kHz, to severe at 10 kHz, and to profound at 20 kHz (FIG. 19). Amikacin has less antibacterial potency and lower intrinsic nephrotoxicity, requiring approximately three-fold higher doses but roughly an equivalent therapeutic index as gentamicin. The loss observed in the gentamicin and amikicin groups, at 100 and 300 mg/kg, respectively, was suggestive of sensorineural loss of cochlear origin. No test positive control article-related middle ear observations were noted in the gross middle ear assessments. However, as described in detail below, histological analysis showed cochlear hair cell loss consistent with the hearing function loss in gentamicin-treated animals.

### Ototoxicity of Compound 1

The ototoxic potential of the Compound 1 was investigated in a separate 28-day study in guinea pigs using the same study design and overlapping dose levels to the gentamicin and amikacin studies described above (executed at MPI Research Inc., Mattawan, Mich.). As in the studies on gentamicin and amikacin, Compound 1 was administered once-daily for 14 days via a bolus subcutaneous (SC) injection between the skin and underlying layers of tissue on the back of each animal, and hearing function changes were assessed by auditory brainstem potentials (ABR) 14 days later. Specifically, three treatment groups of six male and six female Crl:HA (Albino Hartley) guinea pigs were administered the test article at respective dose levels of 8, 30, and 80 mg/kg (Groups 2-4, respectively). One additional group of six male and six female animals served as the control and received the vehicle, 0.9% Sodium chloride for Injection, USP (Group 1). The test article or vehicle was administered to all groups via subcutaneous (SC) injection, once a day for 14 consecutive days, at a dose volume of 1 mL/kg. Following 14 days of administration, all main study animals were maintained for a 16 day recovery period. Additionally, four groups of four male and four female animals were utilized for toxicokinetic (TK) sampling and received the control or test article in the same manner as the main study groups at respective dose levels of 0 (Vehicle Control), 8, 30, or 80 mg/kg.

Observations for morbidity, mortality, injury, and the availability of food and water were conducted at least twice daily for all animals. Clinical observations were conducted prior to the initiation of each dose, following the completion of each dose, daily during the recovery period (Day 15-29), and prior to termination (Day 30). Body weights were measured and recorded daily during the dosing and recovery periods. Physical examinations were conducted pretest on all animals and prior to study termination for main study animals. Auditory brainstem response (ABR) evaluations were performed on main study animals pretest and on Day 29 prior to study termination. Blood samples for determination of the plasma concentrations of the test article were collected from TK animals at designated time points on Day 1 and Day 14. After blood collection, the TK animals were euthanized and the carcasses were discarded. At study termination (Day 30), necropsy examinations were performed and gross middle ear assessments were conducted. Select cochlea, middle ear ossicles, and tympanic membranes were shipped to the Kresge Hearing Research Institute (KHRI) for additional evaluations.

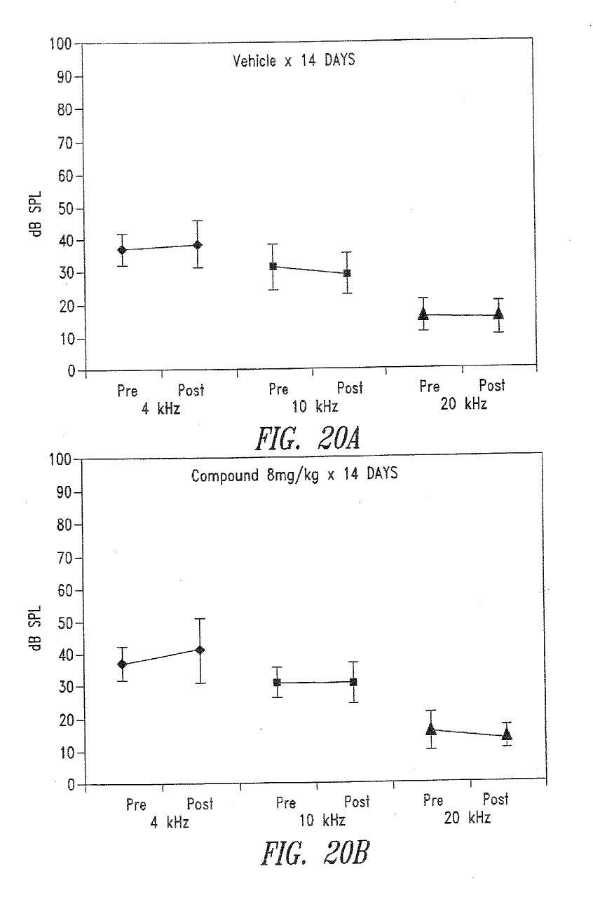

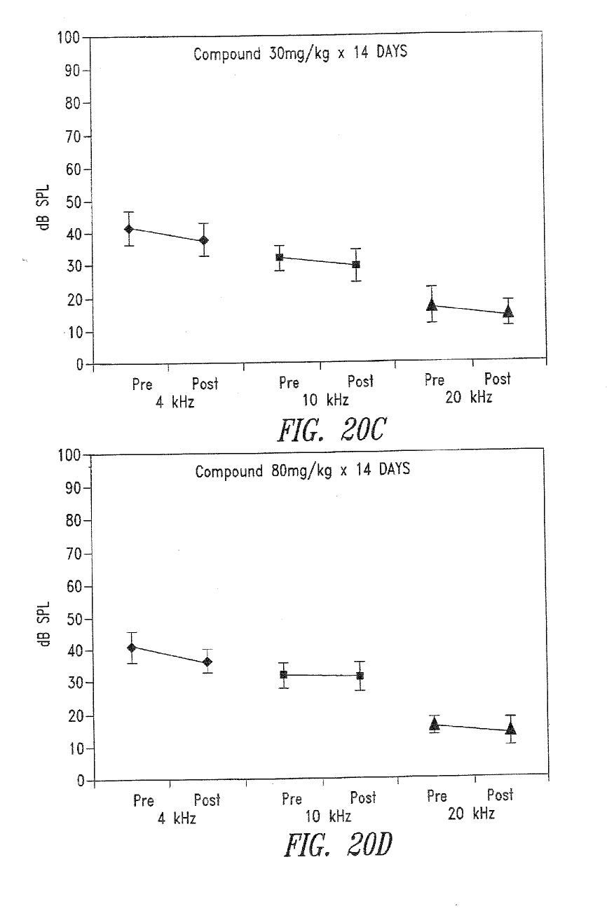

Compound 1 did not produce mortality and was without effect on clinical observations, body weights, physical examinations, ABR evaluations, macroscopic findings, middle ear assessments, or ossicle mobility when administered subcutaneously to a guinea pig. More specifically, Compound 1 did not have an adverse effect on the auditory brainstem evaluations at any dose level (FIG. 20). The average ABR thresholds observed for the pre and post measurements suggested no effect of the treatment for groups 1, 2, 3, or 4. Elevation of thresholds was observed in two male animals in the vehicle control, and in one male and one female in the 8 mg/kg dose group. The threshold elevations were observed at the low frequencies (4 and 10 kHz) and were without loss at the highest frequency (20 kHz). This type of loss is indicative of a conductive loss that is typically associated with the external or middle ear and is not sensorineural in nature. The magnitude of the loss in each case was considered to be mild, and was isolated to the control and low dose treatments. As described in more detail in the next section, there was also no clear evidence that Compound 1 induced hair cell loss upon histological analysis of cochleas.

At the high dose of 80 mg/kg/day, plasma level toxicokinetic analysis demonstrated an average AUC of 370 hr*μg/mL for Compound 1 in the guinea pigs, which corresponds to 1.5× the average AUC observed in the humans at a proposed clinical dose for Compound 1 (approximately 240 hr*μg/mL at 15 mg/kg).

### Cytocochleogram Analysis in 28-day Guinea Pig Ototoxicity Model

These guinea pig ototoxicity studies also included microscopic analysis of surgically dissected cochleas from each animal to assess hair cell damage (cytocochleograms). The underlying cause of aminoglycoside related hearing loss is damage to the hair cells of the auditory system after significant accumulation of aminoglycosides in the inner ear. In the cochlea, drug-induced hair cell loss typically progresses from the basal to the apical turns of the cochlea. Perception of high-frequency sounds, a function of the basal hair cells, is lost first; perception of low-frequency sounds, a function of the more apical hair cells, is the last to be lost (Selimoglu, E., Curr Pharm Des. 2007; 13(1):119-26). For obvious reasons, it is impractical to obtain cytocochleograms from each animal prior to dosing, and some hair cell loss is usually observed in control (vehicle dosed) animals in these guinea pig studies. This variability makes it difficult to determine whether hair cell loss observed post-treatment is due to the drug treatment, especially mild hair cell loss,

Hair cell damage showed a dose response with both gentamicin and amikacin, and hair cell loss was documented at doses below those showing functional hearing loss. When hair cell loss was observed, the pattern of loss was typical of drug-induced ototoxicity, with the greatest loss occurring at the basal end of the cochlea and in the outer hair cells. All animals given gentamicin 100 mg/kg/day showed moderate or large loss of outer hair cells, 4/6 animals given gentamicin 50 mg/kg/day showed minimal or moderate loss of outer hair cells, and one animal given gentamicin 25 mg/kg/day showed minimal levels of outer hair cell loss. All animals given 300 mg/kg/day amikacin showed large loss of outer hair cells, and 3/6 also showed some loss of inner hair cells. At 150 mg/kg/day amikacin, 3/6 animals showed minimal loss of outer hair cells, and at 75 mg/kg/day amikacin, 3/6 animals showed minimal loss of outer hair cells. Thus, the auditory histopathology data showed dose-dependent losses in hair cells in both the gentamicin and amikacin treatment groups. These losses corresponded with auditory brainstem responses in the high dose gentamicin (100 mg/kg) and amikacin (300 mg/kg) groups. These two high dose groups had considerably greater ototoxicity, with a large loss of outer hair cells and some loss of inner hair cells with a corresponding loss of hearing. Specifically, the lowest doses of gentamicin (25 mg/kg) and amikacin (75 mg/kg) produced only small losses of outer hair cells and small hearing loss in few animals. The middle dose of gentamicin (50 mg/kg) resulted in small increases in hair cell loss, and an increase in the number of animals with hair cell loss, but no increase in hearing loss. The middle dose of amikacin (150 mg/kg) did not result in any increase in hair cell loss or the number of animals with hair cell loss, but did result in a small increase in the number of animals (from 1 of 6 to 2 of 6) with hearing loss. Neither middle dose, therefore, provided a large increase in effect over the lower dose. On the other hand, the highest doses of gentamicin (100 mg/kg) and amikacin (300 mg/kg) both showed a dose-response and considerably greater ototoxicity, with a large loss of outer hair cells across animals, and some loss of inner hair cells with a corresponding loss of hearing.

In animals dosed with Compound 1, no dose-response of hair cell loss was apparent. At the high dose level (80 mg/kg) of Compound 1, 2/8 animals showed minimal loss of both inner and outer hair cells at the mid-cochlea or apical end, with no hair cell loss at the basal cochlea. One of 8 animals showed a large loss of outer hair cells across the length of the cochlea, but no loss of inner hair cells. None of the animals treated with Compound 1 at any dose showed any functional hearing loss, which suggests that these findings of sporadic hair cell loss were sub-clinical. More importantly, this pattern of hair cell loss is not typical of drug-induced ototoxicity, and suggests that this hair cell loss was pre-existing or sporadic. At the mid dose level (30 mg/kg) of Compound 1, 3/8 animals showed minimal to moderate outer hair cell loss at the apical end or middle region of the cochlea, and in the low-dose group (8 mg/kg Compound 1) 2/8 animals showed a few spikes of minimal hair cell loss along the length of the cochlea. Again, these patterns of sporadic hair cell loss are not typical of drug-induced hair cell loss. As an illustration of the baseline variability of hair cell patterns in the guinea pig model, ⅛ animals in the vehicle treated group from this study showed minimal hair cell loss along the length of the cochlea.

### Ototoxicity of Short-Course Therapy in Guinea Pigs

The relative ototoxicity of shorter courses of aminoglycoside therapy was also investigated in this guinea pig model. Four treatment groups of six male Crl:HA (Albino Hartley) guinea pigs were administered gentamicin (the test article) at a dose level of 80 mg/kg for 1, 3, 5, or 14 consecutive days (Groups 2-5, respectively). One additional group of six male animals served as the control and received the vehicle, 0.9% Sodium chloride for Injection, USP and was dosed for 14 consecutive days (Group 1). The test article or vehicle was administered to all groups via subcutaneous (SC) injection, once a day for 1 to 14 days, at a dose volume of 1 mL/kg. Following up to 14 days of administration, all main study animals were maintained for a 16 to 29 day recovery period (necropsy on Day 30).

Observations for morbidity, mortality, injury, and the availability of food and water were conducted twice daily for all animals. Clinical observations were conducted prior to the initiation of each dose, following the completion of each dose, and prior to termination (Day 30). Body weights were measured and recorded daily during the dosing and recovery periods. Physical examinations were conducted on all animals pretest and prior to study termination. Auditory brainstem response (ABR) evaluations were performed pretest, once following the completion of dosing (Days 2, 4, 6, or 15 as applicable), and on Day 29 prior to study termination. ABR examinations were conducted at each of three different frequencies (4, 10, and 20 kHz), while animals were anesthetized with a combination of xylazine (2.5 mg/kg) and ketamine (15 mg/kg) and placed in a portable, sound-attenuated, electrically shielded enclosure. Food was withheld from the animals the morning of the ABR procedure and returned following ABR evaluations. At study termination (Day 30), necropsy examinations were performed and gross middle ear assessments were conducted, as described above. The cochlea, middle ear ossicles, and tympanic membranes of each animal were collected for possible future analysis. Cytocochleograms were completed on the cochleas of the left ear of each animal.

Gentamicin administration of varying durations was not associated with any clinical findings over the course of the study, and body weights appeared normal over the course of the study period. Physical examinations appeared normal for the majority of animals. No test article-related middle ear observations were noted in the gross middle ear assessments.

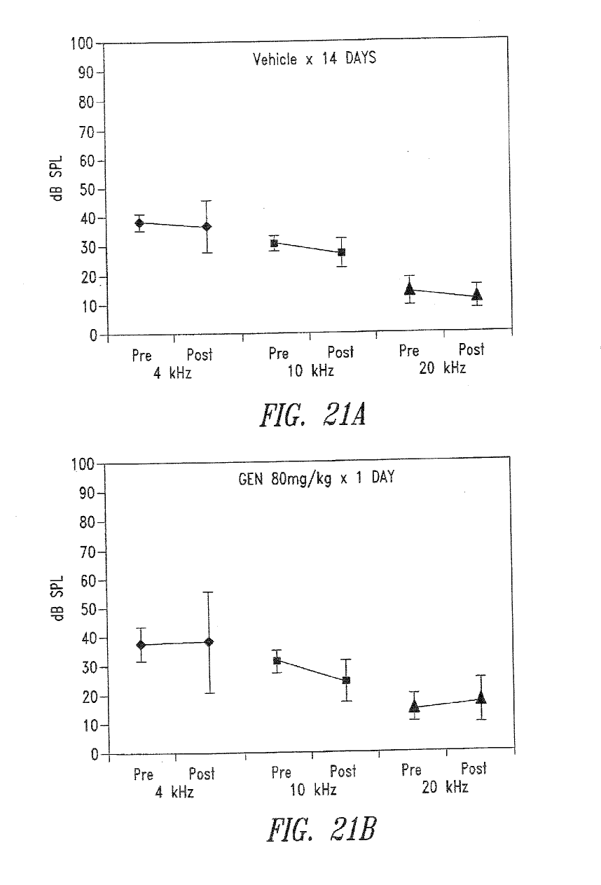

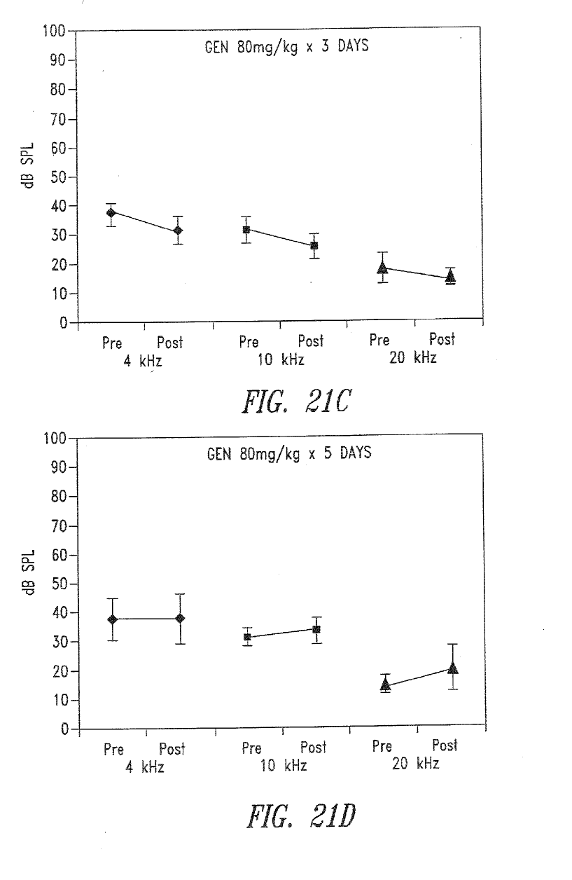

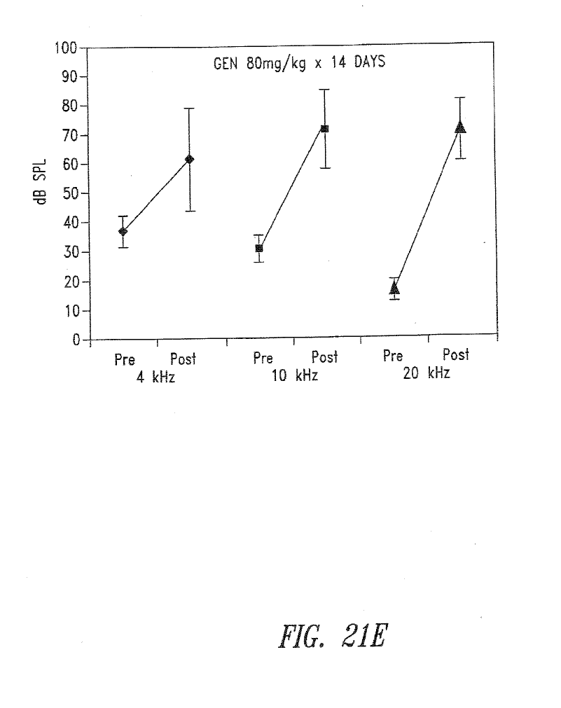

The average Auditory Brainstem Response (ABR) thresholds observed for the pretest and post-exposure intervals suggest no effect of the treatment for Groups 1, 2, 3, and 4 (FIG. 21). Group 5 showed severe to profound hearing loss at the highest test frequencies (10 and 20 kHz) and moderate threshold shifts at 4 kHz in some animals. This type of shift in ABR thresholds in Group 5 animals is consistent with a typical pattern of sensorineural hearing loss. While gentamicin dosed at 80 mg/kg/day for 14 days showed substantial loss of hearing function, gentamicin dosed at 80 mg/kg/day for 1, 3, and 5 days did not show any significant effects on hearing.

Hair cell status was quantitatively assessed in phalloidin stained surface preparations of the cochlear spiral and plotted from apex to base as cytocochleograms. Scatted hair cell loss under 10% and/or a few scattered larger spikes of hair cell loss was considered within the normal variation found across groups of normal untreated animals with normal hearing and was graded as within normal variability. When a few regions and a few rows of OHCs had loss rising above 10% but under 25%, this was considered minimal loss and commonly found in a few subjects in untreated groups. Many regions of loss over 25% was considered a moderate loss, and many regions of loss over 60% was considered a large loss. The results are summarized below.

GROUP 1 (Vehicle Control for Days 1-14)

Five of six animals fell within the normal variability. One animal had a spike of inner hair cell loss.

GROUP 2 (Gentamicin Once on Day 1)

Four of six animals fell within the normal variability, two had slightly above normal variability. One animal had minimal loss of OHCs in row 3 confined to a small region in the apex, and another animal had two small spikes of inner hair cell loss.

GROUP 3 (Gentamicin Daily for Days 1-3)

Four of six animals fell within the normal variability, two had slightly above normal variability. One animal had a small region of increased loss of Outer Hair Cells in row two in mid to basal cochlea and a small spike of inner hair cell loss, and another animal had two minimal spikes of inner hair cell loss.

GROUP 4 (Gentamicin Daily for Days 1-5)

All six animals fell within the normal variability.

GROUP 5 (Gentamicin Daily for Days 1-14)

All six animals had large loss of Outer Hair Cells, and two animals also had large loss of Inner Hair Cells.

These results provide further supportive evidence that the risk of aminoglycoside-induced ototoxicity is decreased by avoiding long durations of treatment. This decreased ototoxicity risk also supports proposed dosing durations of 5 days or less (e.g., 3 days or 5 days) for aminoglycosides, including Compound 1.

### Biological Example 7

### Effect of Infusion Rate on the Accumulation of Gentamicin in Rat Kidney

The effect of the infusion rate of a constant total dose of aminoglycosides on the accumulation of aminoglycosides in kidney and the serum Cₘₐₓ was examined by mathematical modeling.

The accumulation of aminoglycosides in kidney was determined based on a model described in Giuliano, R. A., et al., J Pharmacol Exp Ther 236, 470-475 (1986) and Giuliano, R. A., et al., Am. J. Kidney Dis. 8(5), 297-303 (1986), which indicates that the rate of gentamicin accumulation in rat kidney is a saturable process that is predicted by the Equation 1:

$$Accumulation = \frac{{V}_{ma x} {C}_{P}}{{K}_{m} + {C}_{P}} \qquad ( Equation 1 )$$

where Vₘₐₓ is a maximal velocity for accumulation, Km is an apparent binding constant and CP is the plasma concentration. Based on this model, it was shown in Giuliano, R. A., et al., Am. J. Kidney Dis. 8(5), 297-303 (1986) that once a day bolus dosing resulted in lower kidney accumulation than three times a day bolus dosing (same total dose as in the once a day dosing). These simulated results were in agreement with the experimental results in Giuliano, R. A., et al., Am. J. Kidney Dis. 8(5), 297-303 (1986). In subsequent papers, it was shown that similar results were found for amikacin, tobramycin, gentamicin and netilmicin in man (De Broe, M. E., et al., J Antimicrob Chemother 27 Suppl C, 41-47 (1991); Verpooten, G. A., et al., Clin Pharmacol Ther 45, 22-27 (1989)).

To determine the effect of infusion rate at a constant dose on both aminoglycoside plasma concentration and accumulation of aminoglycoside in kidneys, mathematic modeling was performed to compare the Cₘₐₓ and kidney accumulation concentration of gentamicin infused over 1 minute, 10 minutes, and 30 minutes. Specifically, a serum Cₘₐₓ of 140 μg/ml at 30 min for gentamicin was chosen for the 30 minute infusion. Utilizing the Equation 2 below (Applied Biopharmaceuticals and Pharmacokinetics, 3^{rd} Ed., 1993, Leon Shargel and Andrew B. C. Yu, Appleton and Lang), serum concentrations were predicted:

Cₚ=(R/V_{D}k)(1−e^{-kt}) (Equation 2)

wherein V_{D}=75 ml/kg or 18.7 ml for a standard 250 g rat; R=infusion rate, k=0.035 min⁻¹ (the elimination rate), and Cₚ=plasma concentration. By setting Cₚ=140 μg/mL at t=30 min for the 30 minute infusion, R was calculated to be 141 μg/min resulting in a total dose in 30 min of 4230 μg per 250 g rat (1.68 mg/kg). Adjusting the infusion rate for the 1 minute and 10 minute infusions to always infuse a total dose of 4230 μg, the serum plasma concentrations were calculated using Equation 2 for all three dosing regimes. Then using Equation 1, the kidney accumulations were calculated for the three dosing regimes.

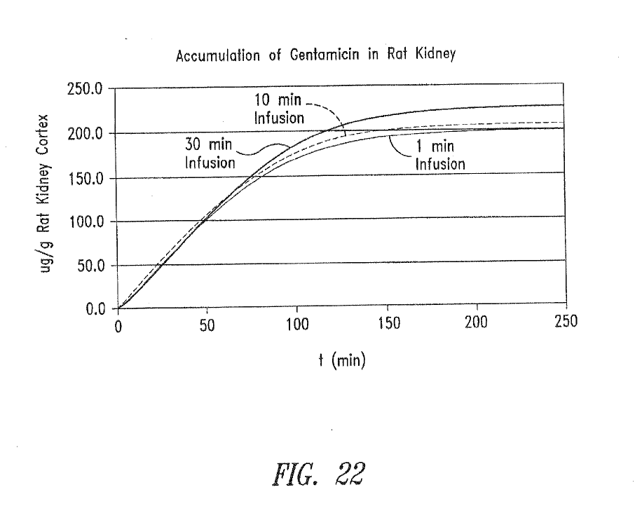

As shown in FIG. 22 and Table 23, according to this mathematical modeling, a faster infusion rate was associated with a higher Cₘₐₓ and a lower accumulation of gentamicin in the kidneys. Accordingly, these data support the use of shorter infusion times for the delivery of aminoglycosides, to enhance efficacy and reduce the potential risk of nephrotoxicity.

TABLE 23
Plasma and Kidney Concentration of Aminoglycoside

| Dosing Scheme | Cmax (1) | Kidney (2) |
|---|---|---|
| 30 min Infusion | 140 | 226 |
| 10 min Infusion | 191 | 207 |
| 1 min Infusion | 223 | 199 |

(1) Plasma Concentration (μg/mL)
(2) Kidney Accumulation Final (μg/g kidney cortex)
(3) Total dose is 16.8 mg/kg

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for treating a bacterial infection in a human subject, the method comprising administering an effective amount of an aminoglycoside to the subject not more than once per day for not more than five days, the effective amount being a potency-normalized amount of at least N_{GEN}×9 mg/kg/day,
where N_{GEN}=MIC_{AG}/MIC_{GEN} is a normalizing factor defined by the ratio of a minimum inhibitory concentration of the administered aminoglycoside, MIC_{AG}, to a minimum inhibitory concentration of gentamicin, MIC_{GEN}.

2. The method of claim 1, wherein the effective amount is also a toxicity-normalized amount of equal to or less than T_{GEN}×50 mg/kg/day,
where T_{GEN}=MTC_{AG}/MTC_{GEN} is a normalizing factor defined by the ratio of a minimum toxic concentration of the administered aminoglycoside, MTC_{AG}, to a minimum toxic concentration of gentamicin, MTC_{GEN}.

3. A method for treating a bacterial infection in a human subject, the method comprising administering an effective amount of an aminoglycoside to the subject not more than once per day for not more than five days, the effective amount being a toxicity normalized amount ranging between T_{GEN}×15 mg/kg/day and T_{GEN}×50 mg/kg/day,
where T_{GEN}=MTC_{AG}/MTC_{GEN} is a normalizing factor defined by the ratio of a minimum toxic concentration of the administered aminoglycoside, MTC_{AG}, to a minimum toxic concentration of gentamicin, MTC_{GEN}.

4-5. (canceled)

6. A method for treating a bacterial infection in a human subject, the method comprising administering an effective amount of an aminoglycoside to the subject not more than once per day for not more than five days to achieve a maximum serum concentration of the administered aminoglycoside, Cₘₐₓ, equal to at least 8 times the minimum inhibitory concentration of the administered aminoglycoside, MIC_{AG}, for the bacteria type infecting the subject.

7. A method for treating a bacterial infection in a human subject, the method comprising administering an effective amount of an aminoglycoside to the subject not more than once per day for not more than five days to achieve a maximum serum concentration of the administered aminoglycoside, Cₘₐₓ, and a pharmacokinetic profile defined by a time-concentration curve, the ratio of Cₘₐₓ to total area under the time-concentration curve, AUC, being at least 0.4 hr⁻¹.

8. (canceled)

9. A method for treating a bacterial infection in a human subject, the method comprising administering an effective amount of an aminoglycoside to the subject not more than once per day for not more than five days to achieve a serum pharmacokinetic profile defined by a time-concentration curve, at least 30% of total area under the time-concentration curve, AUC, being an area above a kidney saturation concentration, C_{KS}, for the aminoglycoside.

10-65. (canceled)

66. The method of claim 1, wherein the aminoglycoside is a compound having the following structure (I): 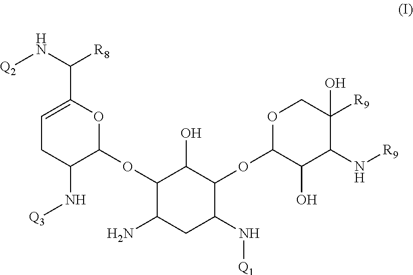
or a stereoisomer, pharmaceutically acceptable salt or prodrug thereof,
wherein:
Q₁ is hydrogen, 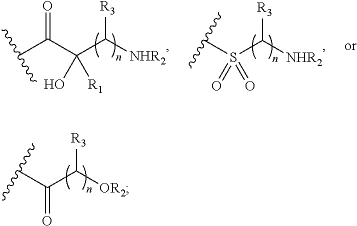
Q₂ is hydrogen, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₄R₅, —(CR₁₀R₁₁)ₚR₁₂, 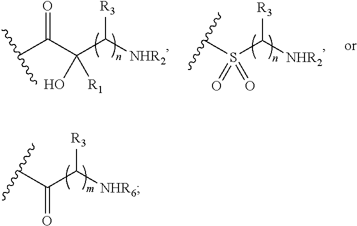
Q₃ is hydrogen, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl, optionally substituted heteroarylalkyl, —C(═NH)NR₄R₅, —(CR₁₀R₁₁)ₚR₁₂, 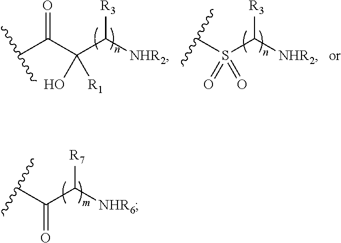
each R₁, R₂, R₃, R₄, R₅, R₈ and R₁₀ is, independently, hydrogen or C₁-C₆ alkyl, or R₁ and R₂ together with the atoms to which they are attached can form a heterocyclic ring having from 4 to 6 ring atoms, or R₂ and R₃ together with the atoms to which they are attached can form a heterocyclic ring having from 4 to 6 ring atoms, or R₁ and R₃ together with the atoms to which they are attached can form a carbocyclic ring having from 4 to 6 ring atoms, or R₄ and R₅ together with the atom to which they are attached can form a heterocyclic ring having from 4 to 6 ring atoms;
each R₆ and R₇ is, independently, hydrogen, hydroxyl, amino or C₁-C₆ alkyl, or R₆ and R₇ together with the atoms to which they are attached can form a heterocyclic ring having from 4 to 6 ring atoms;
each R₉ is, independently, hydrogen or methyl;
each R₁₁ is, independently, hydrogen, hydroxyl, amino or C₁-C₆ alkyl;
each R₁₂ is, independently, hydroxyl or amino;
each n is, independently, an integer from 0 to 4;
each m is, independently, an integer from 0 to 4; and
each p is, independently, an integer from 1 to 5, and
wherein (i) at least two of Q₁, Q₂ and Q₃ are other than hydrogen, and (ii) if Q₁ is hydrogen, then at least one of Q₂ and Q₃ is —C(═NH)NR₄R₅.

67. The method of claim 66 wherein R₈ is hydrogen.

68. The method of claim 66 wherein each R₉ is methyl.

69. The method of claim 66 wherein Q₁ and Q₂ are other than hydrogen.

70. The method of claim 69 wherein Q₃ is hydrogen.

71. The method of claim 69 wherein Q₁ is: 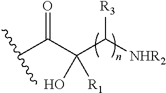
wherein:
R₁ is hydrogen;
R₂ is hydrogen; and
each R₃ is hydrogen.

72. The method of claim 71 wherein Q₁ is:

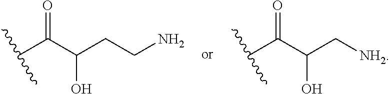

73-80. (canceled)

81. The method of claim 69 wherein Q₂ is —(CR₁₀R₁₁)ₚR₁₂.

82. The method of claim 81 wherein each R₁₀ is hydrogen.

83. The method of claim 81 wherein each R₁₁ is hydrogen.

84-89. (canceled)

90. The method of claim 69 wherein the compound is:
6′-(2-Hydroxy-ethyl)-1-(4-amino-2(S)-hydroxy-butyryl)-sisomicin;
6′-(2-Hydroxy-ethyl)-1-(4-amino-2(R)-hydroxy-butyryl)-sisomicin;
6′-(2-Hydroxy-propanol)-1-(4-amino-2(R)-hydroxy-butyryl)-sisomicin;
6′-(Methyl-piperidin-4-yl)-1-(4-amino-2(R)-hydroxy-butyryl)-sisomicin;
6′-(Methyl-cyclopropyl)-1-(4-amino-2(R)-hydroxy-butyryl)-sisomicin;
6′-(3-Amino-propyl)-1-(4-amino-2(R)-hydroxy-butyryl)-sisomicin;
6′-Methyl-cyclopropyl-1-(3-amino-2(R)-hydroxy-propionyl)-sisomicin;
6′-Methyl-piperidinyl-1-(3-amino-2(R)-hydroxy-propionyl)-sisomicin;
6′-(2-Hydroxy-ethyl)-1-(3-amino-2(R)-hydroxy-propionyl)-sisomicin;
6′-(2-Hydroxy-propanol)-1-(3-amino-2(R)-hydroxy-propionyl)-sisomicin;
6′-(3-Amino-propyl)-1-(3-amino-2(R)-hydroxy-propionyl)-sisomicin;
6′-(Methyl-piperidin-4-yl)-1-(4-amino-2(S)-hydroxy-butyryl)-sisomicin;
6′-(Methyl-cyclopropyl)-1-(3-amino-2(S)-hydroxy-propionyl)-sisomicin;
6′-(2-Hydroxy-propanol)-1-(3-amino-2(S)-hydroxy-propionyl)-sisomicin;
6′-(Methyl-piperidin-4-yl)-1-(3-amino-2(S)-hydroxy-propionyl)-sisomicin;
6′-(2-Hydroxy-ethyl)-1-(3-amino-2(S)-hydroxy-propionyl)-sisomicin;
6′-(3-Amino-propyl)-1-(3-amino-2(S)-hydroxy-propionyl)-sisomicin;
6′-(Methyl-cyclopropyl)-1-(4-amino-2(S)-hydroxy-butyryl)-sisomicin;
6′-(3-Amino-2-hydroxy-propyl)-1-(3-amino-2(S)-hydroxy-propionyl)-sisomicin;
6′-(2-Hydroxy-ethyl)-1-(2-hydroxy-acetyl)-sisomicin;
6′-(3-Amino-propyl)-1-(2-amino-ethylsulfonamide)-sisomicin;
6′-(2-Hydroxy-propanol)-1-(2-amino-ethylsulfonamide)-sisomicin;
6′-(2(S)-Hydroxy-propanol)-1-(4-amino-2(S)-hydroxy-butyryl)-sisomicin;
6′-(2-Hydroxy-ethyl)-1-(2-amino-ethylsulfonamide)-sisomicin;
6′-(Methyl-trans-3-amino-cyclobutyl)-1-(4-amino-2(S)-hydroxy-butyryl)-sisomicin;
6′-(2-Hydroxy-ethyl)-1-(3-hydroxy-pyrrolidin-3-yl-acetyl)-sisomicin;
6′-(2-Hydroxy-4-amino-butyl)-1-(3-hydroxy-pyrrolidin-3-yl-acetyl)-sisomicin;
6′-(Methyl-cyclopropyl)-1-(3-hydroxy-azetidin-3-yl-acetyl)-sisomicin;
6′-(2-Hydroxy-ethyl)-1-(3-hydroxy-azetidin-3-yl-acetyl)-sisomicin;
6′-(Methyl-(1-hydroxy-3-methylamino-cyclobutyl)-1-(4-amino-2(S)-hydroxy-butyryl)-sisomicin;
6′-(3-Amino-propyl)-1-(3-hydroxy-pyrrolidin-3-yl-acetyl)-sisomicin;
6′-(Methyl-cyclopropyl)-1-(3-hydroxy-pyrrolidin-3-yl-acetyl)-sisomicin;
6′-(2-Hydroxy-3-amino-propyl)-1-(3-hydroxy-pyrrolidin-3-yl-acetyl)-sisomicin;
6′-(3-Amino-propyl)-1-(4-amino-2(S)-hydroxy-butyryl)-sisomicin;
6′-(Methyl-pyrrolidin-2-yl)-1-(4-amino-2(S)-hydroxy-butyryl)-sisomicin;
6′-(3-Amino-propyl)-1-(3-hydroxy-azetidin-3-yl-acetyl)-sisomicin;
6′-(3-Amino-propyl)-1-(1-hydroxy-3-amino-cyclobutyl-acetyl)-sisomicin;
6′-(Methyl-trans-3-amino-cyclobutyl)-1-(3-amino-2(S)-hydroxy-propionyl)-sisomicin;
6′-(Methyl-trans-3-amino-cyclobutyl)-1-(1-hydroxy-3-amino-cyclobutyl-acetyl)-sisomicin;
6′-(2-Hydroxy-ethyl)-1-(1-hydroxy-3-amino-cyclobutyl-acetyl)-sisomicin;
6′-Methylcyclopropyl-1-(2-(azetidin-3-yl)-2-hydroxy-acetyl)-sisomicin;
6′-(Methyl-trans-3-amino-cyclobutyl)-1-(2-(azetidin-3-yl)-2-hydroxy-acetyl)-sisomicin;
6′-(2-Hydroxy-ethyl)-1-(2-(azetidin-3-yl)-2-hydroxy-acetyl)-sisomicin;
6′-(3-Amino-propyl)-1-(2-(azetidin-3-yl)-2-hydroxy-acetyl)-sisomicin;
6′-(Methyl-trans-3-amino-cyclobutyl)-1-(3-hydroxy-pyrrolidin-3-yl-acetyl)-sisomicin;
6′-(2-Hydroxy-3-amino-propyl)-1-(2-(azetidin-3-yl)-2-hydroxy-acetyl)-sisomicin; or
6′-(Methyl-3-amino-1-hydroxy-cyclobutyl)-1-(2-(azetidin-3-yl)-2-hydroxy-acetyl)-sisomicin.

91. The method of claim 90 wherein the compound is:
6′-(2-Hydroxy-ethyl)-1-(4-amino-2(S)-hydroxy-butyryl)-sisomicin.

92-175. (canceled)
